(19) United States
(12) Patent Application Publication
(10) Pub. No.: US 2022/0280639 A1
(43) Pub. Date: Sep. 8, 2022

(54) COMPOSITIONS AND METHODS FOR DELIVERY OF RNA INTERFERENCE AGENTS TO IMMUNE CELLS

(71) Applicant: ModernaTX, Inc., Cambridge, MA (US)

(72) Inventor: Eric Yi-Chun HUANG, Boston, MA (US)

(21) Appl. No.: 17/631,216

(22) PCT Filed: Jul. 31, 2020
(86) PCT No.: PCT/US2020/044535

§ 371 Date: Jan. 28, 2022

Related U.S. Application Data
(60) Provisional application No. 62/881,279, filed on Jul. 31, 2019.

(51) Int. Cl. A61K 39/39; A61K 47/14; A61K 9/127; A61K 9/00; A61K 47/24

(52) U.S. Cl. CPC A61K 39/39; A61K 47/14; A61K 9/1272; A61K 9/0019; A61K 47/24; A61K 2039/53

(57) ABSTRACT

The disclosure features lipid nanoparticles (LNPs) comprising RNA interference agents, such as siRNAs, and methods of delivery thereof to immune cells. The compositions and methods can be used to modulate the activity of the immune cells to which the LNPs are delivered, such as to modulate regulatory or effector immune cell activity. Accordingly, the disclosure provides compositions and methods for modulating immune response, for example, to stimulate immune responses, such as in cancer and infectious diseases, or to inhibit immune responses, such as in autoimmune and inflammatory disorders.

### CROSS REFERENCE TO RELATED APPLICATIONS

This application claims the benefit of U.S. Provisional Patent Application No. 62/881,279, filed Jul. 31, 2019, the contents of which is incorporated by reference in its entirety.

### BACKGROUND OF THE DISCLOSURE

The ability to modulate an immune response is beneficial in a variety of clinical situations, including upregulation of immune responses in the treatment of cancer and infections diseases and downregulation of immune responses the treatment of autoimmune diseases, allergies and inflammatory reactions, as well as in prevention of organ transplant rejection and in inhibiting graft-versus-host disease. A number of therapeutic tools exist for modulating the function of biological pathways and/or molecules that are involved in aberrant immune responses. These tools include, for example, small molecules, cytokines, steroids and therapeutic antibodies. However, it can be difficult to control the immunomodulatory effects of such agents, particularly during long-term, systemic administration. For example, a common side effect of many immunosuppressive drugs is immunodeficiency, since the majority of these drugs act non-selectively, resulting in increase susceptibility to infections and decreased cancer immunosurveillance. Additionally, a common side effect of immunostimulatory drugs can be unwanted autoimmune or inflammatory effects.

The RNA interference (RNAi) pathway is also being explored as a route for regulating gene expression as a means to modulate cell activity, such as for therapeutic benefit. For example, RNA interference agents, such as small interfering RNA (siRNA), can be used to prevent mRNA translation to thereby knock down expression of a target protein of interest. However, achieving effective intracellular delivery of siRNA remains a challenge, with ineffective delivery resulting in degradation and/or undesired non-specific effects of the siRNA.

There exists a need in the art for additional effective agents that modulate immune cell activity to thereby modulate immune responses.

### SUMMARY OF THE DISCLOSURE

This disclosure provides lipid nanoparticles (LNPs) comprising RNA interference agents, including small interfering RNAs (siRNAs), that modulate immune cell activity, wherein the LNPs are capable of delivering the RNA interference agent effectively to immune cells. The siRNA can upregulate or downregulate the activity of the immune cell to which it is delivered, to thereby modulate immune responses. In various embodiments, the immune cell can be a T cell (e.g., regulatory T cell, helper T cell, Th7 cell, effector T cell), B cell, NK cell, dendritic cell, myeloid cell or macrophage.

In one embodiment, the lipid nanoparticle comprises a cationic and/or ionizable lipid. In one embodiment, the LNP comprises a sterol or other structural lipid (e.g., a phytosterol or a combination of a phytosterol and cholesterol). In one embodiment, the lipid nanoparticle comprises an immune cell delivery potentiating lipid, which promotes delivery of the RNA interference agent (e.g., siRNA) into immune cells.

Accordingly, in one aspect, the disclosure pertains to a lipid nanoparticle (LNP) for use in a method of immune therapy with enhanced delivery to an immune cell,

wherein the LNP comprises:
(i) a sterol or other structural lipid;
(ii) an ionizable lipid; and
(iii) an RNA interference agent for delivery to an immune cell;

wherein one or more of (i) the sterol or other structural lipid and/or (ii) the ionizable lipid comprises an immune cell delivery potentiating lipid in an amount effective to enhance delivery of the LNP to an immune cell,

wherein the enhanced delivery is a characteristic of said LNP relative to a control LNP lacking the immune cell delivery potentiating lipid.

In one embodiment, the lipid nanoparticle further comprises:
(iv) a non-cationic helper lipid or phospholipid, and/or
(v) a PEG-lipid.

In one embodiment, the sterol or other structural lipid is a phytosterol or cholesterol or combination of a phytosterol and cholesterol. In one embodiment, the sterol or other structural lipid comprises a phytosterol selected from the group consisting of β-sitosterol, stigmasterol, β-sitostanol, campesterol, brassicasterol, and combinations thereof. In one embodiment, the phytosterol is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

In one embodiment, the immune cell delivery potentiating lipid binds to C1q and/or promotes the binding of the LNP comprising said lipid to C1q compared to a control LNP lacking the immune cell delivery potentiating lipid and/or increases uptake of C1q-bound LNP into an immune cell compared to a control LNP lacking the immune cell delivery potentiating lipid.

In one embodiment, the RNA interference agent is a small interfering RNA (siRNA). In one embodiment, the siRNA targets an mRNA encoding a transcription factor in the immune cell. In one embodiment, the siRNA targets an mRNA encoding a soluble protein in the immune cell, such as a cytokine or a chemokine. In one embodiment, the siRNA targets an mRNA encoding an intracellular protein in the immune cell, such as an intracellular adaptor protein or an intracellular signaling molecule. In one embodiment, the siRNA targets an mRNA encoding a membrane-bound protein, such as a receptor on the immune cell.

In one embodiment, the immune cell is a lymphocyte, such as a T cell or a B cell. In other embodiments, the immune cell can be, for example, an NK cell, a dendritic cell, a myeloid cell or a macrophage.

In one embodiment, the RNA interference agent is an siRNA that targets FoxP3 mRNA and the immune cell is a regulatory T cell (Treg). In one embodiment, the RNA interference agent is an siRNA that targets RORc mRNA and the immune cell is a Th17 cell. In one embodiment, the RNA interference agent is an siRNA that targets IL-17a mRNA and the immune cell is a Th17 cell.

In one embodiment, delivery of the LNP to an immune cell results in modulation of activation or activity of the immune cell, such as modulation of activation or activity of a T cell (e.g., Treg cell, T helper cell, Th17 cell, Teff cell), or a B cell, NK cell, dendritic cell, myeloid cell or macrophage.

In another aspect, the disclosure pertains to a pharmaceutical composition comprising a lipid nanoparticle of the disclosure, and a pharmaceutically acceptable carrier, diluent or excipient.

In any of the foregoing or related aspects, the disclosure provides a kit comprising a container comprising a lipid nanoparticle, and an optional pharmaceutically acceptable carrier, or a pharmaceutical composition, and a package insert comprising instructions for administration of the lipid nanoparticle or pharmaceutical composition for modulating an immune response in an individual. In some aspects, the package insert further comprises instructions for administration of the lipid nanoparticle or pharmaceutical composition alone, or in combination with a composition comprising another immunomodulatory agent, and an optional pharmaceutically acceptable carrier for modulating an immune response in an individual.

In any of the foregoing or related aspects, the disclosure provides use of a lipid nanoparticle of the disclosure, and an optional pharmaceutically acceptable carrier, in the manufacture of a medicament for modulating an immune response in an individual, wherein the medicament comprises the lipid nanoparticle and an optional pharmaceutically acceptable carrier and wherein the treatment comprises administration of the medicament, and an optional pharmaceutically acceptable carrier.

In another aspect, the disclosure provides an in vitro method for delivering an RNA interference agent (e.g., siRNA) to an immune cell (e.g., T cell), the method comprising contacting the immune cell with an LNP of the disclosure, which comprises an immune cell delivery potentiating lipid. In one embodiment, the method results in modulation of activation or activity of the immune cell.

In another aspect, the disclosure pertains to a method for modulating an immune response in a subject, the method comprising administering to a subject in need thereof a lipid nanoparticle of the disclosure, or pharmaceutical composition thereof, such that an immune response is modulated in the subject. In one embodiment, modulating an immune response comprises stimulating an immune response in the subject. In another embodiment, modulating an immune response comprises inhibiting an immune response in the subject. In one aspect, modulating an immune response in a subject comprises modulating cytokine production. In another aspect, modulating an immune response in a subject comprises modulating immune cell (e.g., T cell or B cell) proliferation. In another aspect, modulating an immune response in a subject comprises modulating at least one effector function of the immune cell. In another aspect, modulating an immune response in a subject comprises modulating immunoglobulin production (e.g., antigen-specific antibody production).

In any of the foregoing or related aspects, the disclosure provides a method for treating a subject, for example a subject having a disease or condition that would benefit from modulating an immune response in the subject. The treatment method comprises administering to a subject in need thereof any of the foregoing or related immunomodulatory therapeutic compositions or any of the foregoing or related lipid nanoparticle carriers. In some aspects, the immunomodulatory therapeutic composition or lipid nanoparticle carrier is administered in combination with another therapeutic agent (e.g., another immunomodulatory agent).

In one embodiment, the administered nanoparticle results in stimulation of an immune response in the subject, for example when the subject has cancer. Non-limiting examples of types of cancer that can be treated are described herein. In another embodiment of the immunostimulatory methods, the subject has an infectious disease, such as a disease mediated by a viral, bacterial, fungal, yeast or parasitic pathogen. In another embodiment of the immunostimulatory methods, the subject is receiving or has received a vaccine and the method is used to enhance the immune response to the vaccine.

In one embodiment, the administered nanoparticle results in inhibition of an immune response in the subject, for example when the subject has an autoimmune disease, is suspected of having an autoimmune disease or is at risk of developing an autoimmune disease. Non-limiting examples of types of autoimmune diseases that can be treated are described herein. In another embodiment of the immunoinhibitory methods, the subject has an allergic disorder. In another embodiment of the immunoinhibitory methods, the subject has an inflammatory reaction. In another embodiment of the immunoinhibitory methods, the subject is a transplant recipient (e.g., the recipient of a solid organ transplant or a bone marrow transplant, including a subject suffering from GVHD). In another embodiment of the immunoinhibitory methods, the subject is undergoing immunotherapy (e.g., adoptive T cell therapy) and the method is used to downmodulate the immune response that is being stimulated in the subject by the immunotherapy.

In another aspect, the disclosure provides a method of modulating a T cell response in a subject, the method comprising administering to the subject the lipid nanoparticle composition of the disclosure, and an optional pharmaceutically acceptable carrier, such that a T cell response is modulated in the subject. In one embodiment, a T cell response is stimulated in the subject. In one embodiment, a T cell response is inhibited in the subject. In one embodiment, the RNA interference agent is an siRNA. In one embodiment, the siRNA targets mRNA encoding a transcription factor, such as a Foxp3 transcription factor or a ROR transcription factor. In one embodiment, the siRNA targets mRNA encoding a cytokine, such as IL-17a.

In another aspect, the disclosure pertains to an immune cell delivery lipid nanoparticle comprising:

(i) an ionizable lipid;

(ii) a sterol or other structural lipid;

(iii) a non-cationic helper lipid or phospholipid;

(iv) an RNA interference agent, and

(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to an immune cell.

In another aspect, the disclosure pertains to an immune cell delivery lipid nanoparticle comprising:

(i) an ionizable lipid;

(ii) a sterol or other structural lipid;

(iii) a non-cationic helper lipid or phospholipid;

(iv) a PEG-lipid, and

(v) an RNA interference agent,

wherein one or more of (i) the ionizable lipid or (ii) the sterol or other structural lipid comprises an immune cell delivery potentiating lipid in an amount effective to enhance delivery of the lipid nanoparticle to an immune cell.

In yet another aspect, the disclosure pertains to an immune cell delivery lipid nanoparticle comprising:

(i) an ionizable lipid;

(ii) a sterol or other structural lipid;

(iii) a non-cationic helper lipid or phospholipid;

(iv) an RNA interference agent, and

(v) optionally, a PEG-lipid

wherein one or more of (i) the ionizable lipid or (ii) the sterol or other structural lipid or (iii) the non-cationic helper lipid or phospholipid or (v) the PEG lipid is a C1q binding lipid that binds to C1q and/or promotes the binding of the LNP to C1q, as compared to a lipid nanoparticle lacking the C1q binding lipid.

In one embodiment, the enhanced delivery is relative to a lipid nanoparticle lacking the immune cell delivery potentiating lipid. In one embodiment, the enhanced delivery is relative to a suitable control.

In one embodiment, the agent is an siRNA. In one embodiment, the agent is an miRNA. In one embodiment, the agent inhibits expression of a soluble protein (e.g., cytokine) that modulates immune cell activity. In one embodiment, the agent inhibits expression of an intracellular protein (e.g., transcription factor) that modulates immune cell activity. In one embodiment, the agent inhibits expression of a transmembrane protein that modulates immune cell activity. In on embodiment, the agent enhances immune function. In one embodiment, the agent inhibits immune function.

In one embodiment, the immune cell is a T cell. In one embodiment, the immune cell is a B cell. In other embodiments, the immune cell is an NK cell, dendritic cell, myeloid cell or macrophage.

In one embodiment, the immune cell delivery lipid nanoparticle comprises a phytosterol or a combination of a phytosterol and cholesterol. In one embodiment, the phytosterol is selected from the group consisting of β-sitosterol, stigmasterol, β-sitostanol, campesterol, brassicasterol, and combinations thereof. In one embodiment, the phytosterol comprises a sitosterol or a salt or an ester thereof. In one embodiment, the phytosterol comprises a stigmasterol or a salt or an ester thereof. In one embodiment, the phytosterol is beta-sitosterol

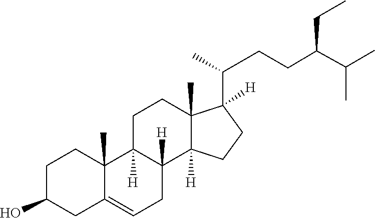

or a salt or an ester thereof.

In one embodiment, the immune cell delivery lipid nanoparticle comprises a phytosterol, or a salt or ester thereof, and cholesterol or a salt thereof. In one embodiment, the immune cell is a T cell and the phytosterol or a salt or ester thereof is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof. In one embodiment, the phytosterol is β-sitosterol. In one embodiment, the phytosterol is β-sitostanol. In one embodiment, the phytosterol is campesterol. In one embodiment, the phytosterol is brassicasterol.

In one embodiment, the immune cell is a monocyte or a myeloid cell and the phytosterol or a salt or ester thereof is selected from the group consisting of β-sitosterol, and stigmasterol, and combinations thereof. In one embodiment, the phytosterol is β-sitosterol. In one embodiment, the phytosterol is stigmasterol.

In one embodiment, the immune cell delivery lipid nanoparticle comprises a sterol, or a salt or ester thereof, and cholesterol, wherein the immune cell is a monocyte or a myeloid cell and the sterol or a salt or ester thereof is selected from the group consisting of β-sitosterol-d7, brassicasterol, Compound S-30, Compound S-31 and Compound S-32. In one embodiment, the immune cell delivery lipid nanoparticle comprises a sterol, or a salt or ester thereof, and cholesterol, wherein the immune cell is a monocyte or a myeloid cell and the sterol or a salt or ester thereof is selected from the group consisting of brassicasterol, Compound S-30, Compound S-31 and Compound S-32.

In one embodiment, the immune cell delivery lipid nanoparticle comprises cholesterol and a phytosterol, wherein the mol % cholesterol is between about 1% and 50% of the mol % of phytosterol present in the lipid nanoparticle. In one embodiment, the mol % cholesterol is between about 10% and 40% of the mol % of phytosterol present in the lipid nanoparticle. In one embodiment, the mol % cholesterol is between about 20% and 30% of the mol % of phytosterol present in the lipid nanoparticle. In one embodiment, the mol % cholesterol is about 30% of the mol % of phytosterol present in the lipid nanoparticle.

In one embodiment of the immune cell delivery lipid nanoparticle, the ionizable lipid comprises a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8). In one embodiment, the ionizable lipid comprises a compound selected from the group consisting of Compound X, Compound Y, Compound I-48, Compound I-50, Compound I-109, Compound I-111, Compound I-113, Compound I-181, Compound I-182, Compound I-244, Compound I-292, Compound I-301, Compound I-309, Compound I-317, Compound I-321, Compound I-322, Compound I-326, Compound I-328, Compound I-330, Compound I-331, Compound I-332, Compound I-347, Compound I-348, Compound I-349, Compound I-350, Compound I-352 and Compound I-M. In one embodiment, the ionizable lipid comprises a compound selected from the group consisting of Compound X, Compound Y, Compound I-321, Compound I-292, Compound I-326, Compound I-182, Compound I-301, Compound I-48, Compound I-50, Compound I-328, Compound I-330, Compound I-109, Compound I-111 and Compound I-181.

In one embodiment, the immune cell is a T cell (e.g., a Treg cell or a Teff cell, such as a Th17 cell). In one embodiment, the immune cell is a T cell and the ionizable lipid comprises a compound selected from the group consisting of Compound I-301, Compound I-321, and Compound I-326.

In one embodiment, the immune cell is a monocyte or a myeloid cell and the ionizable lipid comprises a compound selected from the group consisting of Compound X, Compound I-109, Compound I-111, Compound I-181, Compound I-182, and Compound I-244.

In one embodiment, the non-cationic helper lipid or phospholipid comprises a compound selected from the group consisting of DSPC, DPPC, DMPC, DMPE, DOPC, Compound H-409, Compound H-418, Compound H-420, Compound H-421 and Compound H-422. In one embodiment, the phospholipid is DSPC. In one embodiment, the immune cell is a T cell and the non-cationic helper lipid or phospholipid comprises a compound selected from the group consisting of DSPC, DMPE, and Compound H-409. In one embodiment, the immune cell is a T cell and the phospholipid is DSPC. In one embodiment, the immune cell is a T cell and the phospholipid is DMPE. In one embodiment, the immune cell is a T cell and the phospholipid is Compound H-409. In one embodiment, the immune cell is a monocyte or a myeloid cell and the non-cationic helper lipid or phospholipid comprises a compound selected from the group consisting of DOPC, DMPE, and Compound H-409. In one embodiment, the immune cell is a monocyte or myeloid cell and the phospholipid is DOPC. In one embodiment, the immune cell is a monocyte or myeloid cell and the phospholipid is DMPE. In one embodiment, the immune cell is a monocyte or myeloid cell and the phospholipid is Compound H-409.

In one embodiment, the immune cell delivery lipid nanoparticle comprises a PEG-lipid. In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In one embodiment, the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25. In one embodiment, the immune cell is a T cell and the PEG lipid comprises a compound selected from the group consisting of Compound P-428, Compound P-L16, Compound P-L17, Compound P-L18, Compound P-L19, Compound P-L1, and Compound P-L2.

In one embodiment, the immune cell delivery lipid nanoparticle comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid. In one embodiment, the immune cell delivery lipid nanoparticle comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid. In one embodiment, the immune cell delivery lipid nanoparticle comprises about 50 mol % ionizable lipid, about 10 mol % non-cationic helper lipid or phospholipid, about 38.5 mol % sterol or other structural lipid, and about 1.5 mol % PEG lipid. In one embodiment of the immune cell delivery lipid nanoparticle, the mol % sterol or other structural lipid is 18.5% phytosterol and the total mol % structural lipid is 38.5%. In one embodiment of the immune cell delivery lipid nanoparticle, the mol % sterol or other structural lipid is 28.5% phytosterol and the total mol % structural lipid is 38.5%. In one embodiment, the immune cell is a T cell (e.g., Treg cell or Teff cell, such as Th17 cell).

In one embodiment, the immune cell delivery lipid nanoparticle comprises:

(i) about 50 mol % ionizable lipid, wherein the ionizable lipid is a compound selected from the group consisting of Compound I-301, Compound I-321, and Compound I-326;

(ii) about 10 mol % phospholipid, wherein the phospholipid is DSPC;

(iii) about 38.5 mol % structural lipid, wherein the structural lipid is selected from β-sitosterol and cholesterol; and

(iv) about 1.5 mol % PEG lipid, wherein the PEG lipid is Compound P-428.

In one embodiment of the immune cell delivery lipid nanoparticle the RNA interference agent comprises at least one modified nucleobase, nucleoside and/or nucleotide.

In one embodiment of the immune cell delivery lipid nanoparticle, the immune cell is a Treg cell. In one embodiment, the RNA interference agent is an siRNA. In one embodiment, the RNA interference agent is an miRNA. In one embodiment, the RNA interference agent is an siRNA that targets an mRNA encoding Foxp3. In one embodiment, the RNA interference agent (e.g., siRNA) targets an mRNA encoding a protein selected from the group consisting of Foxp3, IRF4, estrogen receptor 1, HDAC6, HDAC10, HDAC11 and AEP. In one embodiment, the RNA interference agent (e.g., siRNA) targets miR-146b or anti-miR-146b.

In one embodiment of the immune cell delivery lipid nanoparticle, the immune cell is a Teff cell. In one embodiment, the RNA interference agent is an siRNA. In one embodiment, the RNA interference agent is an miRNA. In one embodiment, the Teff cell is a Th17 cell. In one embodiment, the RNA interference agent is an siRNA that targets an mRNA encoding RORγt or IL-17a. In one embodiment, the RNA interference agent (e.g., siRNA) targets an mRNA encoding a protein selected from the group consisting of RORγt, IL-17a, Tbet, Kv1.3, KCA3.1 and KCNNA.

In another aspect, the disclosure pertains to a method of delivering an agent to an immune cell, the method comprising contacting the immune cell with an immune cell delivery lipid nanoparticle comprising:

(i) an ionizable lipid;

(ii) a sterol or other structural lipid;

(iii) a non-cationic helper lipid or phospholipid;

(iv an RNA interference agent, and

(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to an immune cell,

such that the agent is delivered to the immune cell.

In another aspect, the disclosure pertains to a method of modulating T cell activation or activity, the method comprising contacting a T cell with an immune cell delivery lipid nanoparticle comprising:

(i) an ionizable lipid;

(ii) a sterol or other structural lipid;

(iii) a non-cationic helper lipid or phospholipid;

(iv) an RNA interference agent, and

(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to a T cell,

such that T cell activation or activity is modulated.

In one embodiment, the T cell is a Treg cell. In one embodiment, the T cell is a Teff cell. In one embodiment, the Teff cell is a Th17 cell.

In another aspect, the disclosure pertains to a method of increasing an immune response to a protein, the method comprising contacting immune cells with an immune cell delivery lipid nanoparticle comprising:

(i) an ionizable lipid;

(ii) a sterol or other structural lipid;

(iii) a non-cationic helper lipid or phospholipid;

(iv) an RNA interference agent, and

(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to immune cells,

such that the immune response to the protein is increased.

In another aspect, the disclosure pertains to a method of increasing a T cell response to a cancer antigen, the method comprising contacting the T cell with an immune cell delivery lipid nanoparticle comprising:

(i) an ionizable lipid;

(ii) a sterol or other structural lipid;

(iii) a non-cationic helper lipid or phospholipid;

(iv) an RNA interference agent, and

(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to immune cells,

such that the T cell response to the cancer antigen is increased.

In another aspect, the disclosure pertains to a method of modulating an immune response in a subject, the method comprising administering to the subject an immune cell delivery lipid nanoparticle comprising:

(i) an ionizable lipid;

(ii) a sterol or other structural lipid;

(iii) a non-cationic helper lipid or phospholipid;

(iv) an RNA interference agent, and

(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to immune cells,

such that an immune response is modulated in the subject.

In another aspect, the disclosure pertains to a method of modulating B cell activation or activity, the method comprising contacting a B cell with an immune cell delivery lipid nanoparticle comprising:

(i) an ionizable lipid;

(ii) a sterol or other structural lipid;

(iii) a non-cationic helper lipid or phospholipid;

(iv) an RNA interference agent, and

(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to immune cells,

such that B cell activation or activity is modulated.

In one embodiment of the methods, the enhanced delivery is relative to a lipid nanoparticle lacking the immune cell delivery potentiating lipid. In one embodiment, the enhanced delivery is relative to a suitable control.

In one embodiment of the methods, the agent is an siRNA. In one embodiment, the agent is an miRNA. In one embodiment, the agent inhibits expression of a soluble protein (e.g., cytokine) that modulates immune cell activity. In one embodiment, the agent inhibits expression of an intracellular protein (e.g., transcription factor) that modulates immune cell activity. In one embodiment, the agent inhibits expression of a transmembrane protein that modulates immune cell activity. In on embodiment, the agent enhances immune function. In one embodiment, the agent inhibits immune function.

In one embodiment of the methods, the immune cell is a T cell. In one embodiment, the immune cell is a B cell. In other embodiments, the immune cell is an NK cell, dendritic cell, myeloid cell or macrophage.

In one embodiment of the methods, the immune cell delivery lipid nanoparticle comprises a phytosterol or a combination of a phytosterol and cholesterol. In one embodiment, the phytosterol is selected from the group consisting of β-sitosterol, stigmasterol, β-sitostanol, campesterol, brassicasterol, and combinations thereof. In one embodiment, the phytosterol comprises a sitosterol or a salt or an ester thereof. In one embodiment, the phytosterol comprises a stigmasterol or a salt or an ester thereof. In one embodiment, the phytosterol is beta-sitosterol

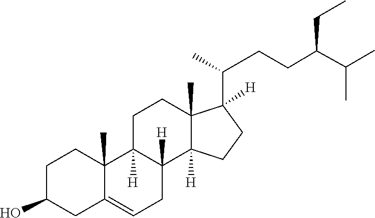

or a salt or an ester thereof.

In one embodiment of the methods, the immune cell delivery lipid nanoparticle comprises a phytosterol, or a salt or ester thereof, and cholesterol or a salt thereof. In one embodiment, the immune cell is a T cell and the phytosterol or a salt or ester thereof is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof. In one embodiment, the phytosterol is β-sitosterol. In one embodiment, the phytosterol is β-sitostanol. In one embodiment, the phytosterol is campesterol. In one embodiment, the phytosterol is brassicasterol.

In one embodiment of the methods, the immune cell is a monocyte or a myeloid cell and the phytosterol or a salt or ester thereof is selected from the group consisting of β-sitosterol, and stigmasterol, and combinations thereof. In one embodiment, the phytosterol is β-sitosterol. In one embodiment, the phytosterol is stigmasterol.

In one embodiment of the methods, the immune cell delivery lipid nanoparticle comprises a sterol, or a salt or ester thereof, and cholesterol, wherein the immune cell is a monocyte or a myeloid cell and the sterol or a salt or ester thereof is selected from the group consisting of β-sitosterol-d7, brassicasterol, Compound S-30, Compound S-31 and Compound S-32. In one embodiment of the methods, the immune cell delivery lipid nanoparticle comprises a sterol, or a salt or ester thereof, and cholesterol, wherein the immune cell is a monocyte or a myeloid cell and the sterol or a salt or ester thereof is selected from the group consisting of brassicasterol, Compound S-30, Compound S-31 and Compound S-32.

In one embodiment of the methods, the immune cell delivery lipid nanoparticle comprises cholesterol and a phytosterol, wherein the mol % cholesterol is between about 1% and 50% of the mol % of phytosterol present in the lipid nanoparticle. In one embodiment, the mol % cholesterol is between about 10% and 40% of the mol % of phytosterol present in the lipid nanoparticle. In one embodiment, the mol % cholesterol is between about 20% and 30% of the mol % of phytosterol present in the lipid nanoparticle. In one embodiment, the mol % cholesterol is about 30% of the mol % of phytosterol present in the lipid nanoparticle.

In one embodiment of the methods, the ionizable lipid comprises a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8). In one embodiment, the ionizable lipid comprises a compound selected from the group consisting of Compound X, Compound Y, Compound I-48, Compound I-50, Compound I-109, Compound I-111, Compound I-113, Compound I-181, Compound I-182, Compound I-244, Compound I-292, Compound I-301, Compound I-309, Compound I-317, Compound I-321, Compound I-322, Compound I-326, Compound I-328, Compound I-330, Compound I-331, Compound I-332, Compound I-347, Compound I-348, Compound I-349, Compound I-350, Compound I-352 and Compound I-M. In one embodiment, the ionizable lipid comprises a compound selected from the group consisting of Compound X, Compound Y, Compound I-321, Compound I-292, Compound I-326, Compound I-182, Compound I-301, Compound I-48, Compound I-50, Compound I-328, Compound I-330, Compound I-109, Compound I-111 and Compound I-181.

In one embodiment of the methods, the immune cell is a T cell (e.g., a Treg cell or a Teff cell, such as a Th17 cell). In one embodiment, the immune cell is a T cell and the ionizable lipid comprises a compound selected from the group consisting of Compound I-301, Compound I-321, and Compound I-326.

In one embodiment of the methods, the immune cell is a monocyte or a myeloid cell and the ionizable lipid comprises a compound selected from the group consisting of Compound X, Compound I-109, Compound I-111, Compound I-181, Compound I-182, and Compound I-244.

In one embodiment of the methods, the non-cationic helper lipid or phospholipid comprises a compound selected from the group consisting of DSPC, DPPC, DMPC, DMPE, DOPC, Compound H-409, Compound H-418, Compound H-420, Compound H-421 and Compound H-422. In one embodiment, the phospholipid is DSPC. In one embodiment, the immune cell is a T cell and the non-cationic helper lipid or phospholipid comprises a compound selected from the group consisting of DSPC, DMPE, and Compound H-409. In one embodiment, the immune cell is a T cell and the phospholipid is DSPC. In one embodiment, the immune cell is a T cell and the phospholipid is DMPE. In one embodiment, the immune cell is a T cell and the phospholipid is Compound H-409. In one embodiment, the immune cell is a monocyte or a myeloid cell and the non-cationic helper lipid or phospholipid comprises a compound selected from the group consisting of DOPC, DMPE, and Compound H-409. In one embodiment, the immune cell is a monocyte or myeloid cell and the phospholipid is DOPC. In one embodiment, the immune cell is a monocyte or myeloid cell and the phospholipid is DMPE. In one embodiment, the immune cell is a monocyte or myeloid cell and the phospholipid is Compound H-409.

In one embodiment of the methods, the immune cell delivery lipid nanoparticle comprises a PEG-lipid. In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In one embodiment, the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25. In one embodiment, the immune cell is a T cell and the PEG lipid comprises a compound selected from the group consisting of Compound P-428, Compound P-L16, Compound P-L17, Compound P-L18, Compound P-L19, Compound P-L1, and Compound P-L2.

In one embodiment of the methods, the immune cell delivery lipid nanoparticle comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid. In one embodiment, the immune cell delivery lipid nanoparticle comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid. In one embodiment, the immune cell delivery lipid nanoparticle comprises about 50 mol % ionizable lipid, about 10 mol % non-cationic helper lipid or phospholipid, about 38.5 mol % sterol or other structural lipid, and about 1.5 mol % PEG lipid. In one embodiment of the immune cell delivery lipid nanoparticle, the mol % sterol or other structural lipid is 18.5% phytosterol and the total mol % structural lipid is 38.5%. In one embodiment of the immune cell delivery lipid nanoparticle, the mol % sterol or other structural lipid is 28.5% phytosterol and the total mol % structural lipid is 38.5%. In one embodiment, the immune cell is a T cell (e.g., Treg cell or Teff cell, such as Th17 cell).

In one embodiment of the methods, the immune cell delivery lipid nanoparticle comprises:

(i) about 50 mol % ionizable lipid, wherein the ionizable lipid is a compound selected from the group consisting of Compound I-301, Compound I-321, and Compound I-326;

(ii) about 10 mol % phospholipid, wherein the phospholipid is DSPC;

(iii) about 38.5 mol % structural lipid, wherein the structural lipid is selected from β-sitosterol and cholesterol; and

(iv) about 1.5 mol % PEG lipid, wherein the PEG lipid is Compound P-428.

In one embodiment of the methods, the RNA interference agent comprises at least one modified nucleobase, nucleoside and/or nucleotide.

In one embodiment of the methods, the immune cell is a Treg cell. In one embodiment, the RNA interference agent is an siRNA. In one embodiment, the RNA interference agent is an miRNA. In one embodiment, the RNA interference agent is an siRNA that targets an mRNA encoding Foxp3. In one embodiment, the RNA interference agent (e.g., siRNA) targets an mRNA encoding a protein selected from the group consisting of Foxp3, IRF4, estrogen receptor 1, HDAC6, HDAC10, HDAC11 and AEP. In one embodiment, the RNA interference agent (e.g., siRNA) targets miR-146b or anti-miR-146b.

In one embodiment of the methods, the immune cell is a Teff cell. In one embodiment, the RNA interference agent is an siRNA. In one embodiment, the RNA interference agent is an miRNA. In one embodiment, the Teff cell is a Th17 cell. In one embodiment, the RNA interference agent is an siRNA that targets an mRNA encoding RORγt or IL-17a. In one embodiment, the RNA interference agent (e.g., siRNA) targets an mRNA encoding a protein selected from the group consisting of RORγt, IL-17a, Tbet, Kv1.3, KCA3.1 and KCNNA.

### BRIEF DESCRIPTION OF THE DRAWINGS

FIGS. 1A-1C are histograms showing alteration of Foxp3 expression in in vitro differentiated mouse regulatory T cells (Tregs) using three different commercial sources of small interfering RNA (siRNA) constructs against Foxp3 formulated in LNPs. FIGS. 1A-1B show results for differentiated Tregs incubated with LNP-encapsulated control or Foxp3 siRNA for either 24 hr (FIG. 1A) or 48 hr (FIG. 1B). FIG. 1C shows results for differentiated Tregs incubated with LNP-encapsulated control or Foxp3 siRNA for 24 hr, and then washed and refreshed with media for an additional 24 hr. The graphs show the mean fluorescence intensity (MFI) of Foxp3 within the live CD4+ T cell population.

FIGS. 2A-2C are histograms showing alteration of Foxp3 expression in cultured mouse splenocytes (FIG. 2A), differentiated mouse Tregs (FIG. 2B), or ex vivo mouse Tregs (FIG. 2C) incubated in vitro with LNPs encapsulating the single siRNA construct from Vendor 1 against Foxp3. Splenocytes were cultured for 24 hr with 10 μg/ml or 1 μg/ml siRNA (10× and 1×, respectively; FIG. 2A). Differentiated Tregs and ex vivo Tregs were cultured for 24 hr with 1 μg/ml siRNA (FIGS. 2B and 2C). Control=scrambled siRNA.

FIGS. 3A-3B are histograms showing alteration of Foxp3 expression in differentiated mouse Tregs in vitro by 5-fold dilutions of LNPs encapsulating the single siRNA construct from Vendor 1 against Foxp3 (FIG. 3A) or control siRNA (FIG. 3B).

FIGS. 4A-4B are histograms showing disruption of mouse Treg differentiation expression by 5-fold dilutions of LNPs encapsulating the single siRNA construct from Vendor 1 against Foxp3. Naïve mouse CD4+ T cells were cultured in Treg differentiation conditions for 6d (FIG. 4A) or 7d (FIG. 4B). The indicated siRNA was added to the cultures at the start of differentiation.

FIG. 5 is a graph showing the proliferation of effector CD4+CD25− T cells (Teff) cultured with differentiated Tregs that were incubated with LNPs encapsulating control or Foxp3 siRNA. The x-axis shows the Treg:Teff ratio. The y-axis shows the percentage of proliferated Teff cells. The dotted line represents the amount of proliferation with no stimulation.

FIGS. 6A-6B are graphs of differentiated mouse Th17 cells cultured with serial dilutions of LNPs encapsulating siRNA pools against RAR related orphan protein receptor C (RORc), interleukin-17a (IL-17a), or scrambled siRNA. Differentiated Th17 cells were cultured with siRNA for 24 h (FIG. 6A) or 48 h (FIG. 6B) and then stimulated with phorbol 12-myristate 13-acetate (PMA), ionomycin, and brefeldin A for 6 h to amplify intracellular cytokine signal. Represented are the MFI of IL-17a within the live CD4+ T cell population. The dotted line represents IL-17a MFI of cells that did not receive siRNA.

### DETAILED DESCRIPTION

The disclosure provides lipid nanoparticles (LNPs) encapsulating an RNA interference agent (e.g., siRNA), wherein the LNPs comprise an immune cell delivery potentiating lipid in an amount effective to enhance delivery of the LNP to an immune cell to thereby deliver the RNA interference agent into the immune cell. The disclosure further provides methods of using the LNPs in vitro and in vivo to deliver an RNA interference agent (e.g., siRNA) into an immune cell. The disclosure further provides methods of modulating immune cell activity, and thereby modulating immune responses, using the LNPs of the disclosure.

As demonstrated in the examples, siRNAs (single constructs or pooled) have been encapsulated in LNPs that comprise an immune cell delivery potentiating lipid, and these formulations have been demonstrated to deliver the siRNA into immune cells (e.g., splenocytes, Treg cells) such that the mRNA targeted by the siRNA is downregulated in the immune cells (see e.g., Examples, 1-3 and 6). Furthermore, delivery of the LNP-encapsulated siRNA into immune cells was demonstrated to modulate the differentiation of the immune cells (see Example 4). Moreover, delivery of the LNP-encapsulated siRNA into immune cells was demonstrated to modulate the functional activity of the immune cells (see Example 5).

In addition to the RNA interference agent, the LNPs of the disclosure typically comprise a sterol or other structural lipid and an ionizable lipid, wherein either or both of the sterol/structural lipid and the ionizable lipid comprise an immune cell delivery potentiating lipid in an amount effective to enhance delivery of the LNP to an immune cell, wherein the enhanced delivery is a characteristic of said LNP relative to a control LNP lacking the immune cell delivery potentiating lipid. In certain embodiments, the LNP can further comprise a non-cationic helper lipid or phospholipid and/or a PEG lipid.

The various components of the LNPs, and methods of use thereof, are described in detail in the subsections below.

### RNA Interference Agents

RNA interference (RNAi) refers to a biological process in which RNA molecules inhibit gene expression or translation by neutralizing targeted mRNA molecules. RNAi is a gene silencing process that is controlled by the RNA-induced silencing complex (RISC) and is initiated by short double-stranded RNA molecules (dsRNA) in a cell's cytoplasm. Two types of small ribonucleic acid molecules, small interfering RNAs (siRNAs) and microRNAs (miRNAs), are central to RNA interference. While RNAi is a natural cellular process, the components of RNAi also have been synthesized and exploited for inhibiting expression of target genes/mRNAs of interest in vitro and in vivo.

As a natural process, dsRNA initiates RNAi by activating the ribonuclease protein Dicer, which binds and cleaves dsRNA and short hairpin RNAs (shRNAs) to produce double-stranded fragments of 20-25 base pairs. These short double-stranded fragments are called small interfering RNAs (siRNAs). These siRNAs are then separated into single strands and integrated into an active RISC, by the RISC-Loading Complex (RLC). After integration into the RISC, siRNAs base-pair to their target mRNA and cleave it, thereby preventing it from being used as a translation template.

The phenomenon of RNAi, broadly defined, also includes the gene silencing effects of miRNAs. MicroRNAs are genetically-encoded non-coding RNAs that help regulate gene expression, for example during development. Naturally-occurring mature miRNAs are structurally similar to siRNAs produced from exogenous dsRNA, but before reaching maturity, miRNAs undergo extensive post-transcriptional modification, including a dsRNA portion of pre-miRNA being cleaved by Dicer to produce the mature miRNA molecule that can be integrated into the RISC complex.

Accordingly, while typically the RNA interference agent encapsulated by the LNPs of the disclosure is an siRNA, any agent that mediates or is involved in the RNA interference (RNAi) process can be used as an RNA interference agent, including siRNAs and miRNAs, each of which is described in further detail below.

RNA interference agents, including siRNAs and miRNAs, are commercially available in the art, including custom design and synthesis (e.g., Dharmacon, ThermoFisher Scientific) and/or can be synthesized by standard methods well established in the art. Additionally, RNA interference agents, including siRNAs and miRNAs, can be chemically modified to enhance their properties (e.g., therapeutic properties), as has been described in the art. For example, chemically modified siRNAs known in the art are described in detail in the database at the website http://crdd.osdd.net/servers/sirnamod/. The SiRNAmod database of experimentally validated chemically modified siRNAs is also described in Dar, S. A. et al. (2016) Scientific Reports 6:20031. Synthesis and modification of RNA interference agents is described in further detail below.

Small Interfering RNAs

Small interfering RNAs (siRNAs), also referred to as short interfering RNAs or silencing RNAs, are a class of double-stranded RNA molecules, typically 20-25 base pairs in length, that operate within the RNAi pathway to interfere with the expression of specific target sequences with complementary nucleotide sequences. siRNAs inhibit gene expression by degrading mRNA after transcription, thereby preventing translation. As used herein, the term “siRNA” encompasses all forms of siRNAs known in the art, including, but not limited to, shortmers, longmers, 2′5′-isomers and Dicer-substrate RNAs. Naturally-occurring and artificially synthesized siRNAs, and their use in therapy (e.g., delivered by nanoparticles), have been described in the art (see e.g., Hamilton and Balcombe (1999) Science 286:950-952; Elbashir et al. (2001) Nature 411:494-498; Shen et al. (2012) Cancer Gene Therap. 19:367-373; Wittrup et al. (2015) Nat. Rev. Genet. 16:543-552).

Accordingly, in one embodiment, the RNA interference agent associated with/encapsulated by the lipid-based composition, e.g., LNP, is an siRNA. In some embodiments, a pool of siRNA is associated with/encapsulated by the lipid-based composition. In some embodiments, a pool of siRNA is more than one siRNA targeting the same gene. In some embodiments, a pool of siRNA is two, three or four different siRNA targeting the same gene. In some embodiments, a pool of siRNA further decreases expression of a target gene compared to an individual siRNA. In some embodiments, a pool of siRNA decreases expression of a target gene by at least 10%, 20%, 30%, 40%, 50%, 60%, 70%, 80% or 90% more compared to an individual siRNA.

In one embodiment, the siRNA inhibits expression of a target sequence expressed in immune cells. In one embodiment, the siRNA inhibits expression of a target sequence expressed in lymphoid cells. In one embodiment, the siRNA inhibits expression of a target sequence expressed in T cells. In one embodiment, the siRNA inhibits expression of a target sequence expressed in B cells. In one embodiment, the siRNA inhibits expression of a target sequence expressed in NK cells. In one embodiment, the siRNA inhibits expression of a target sequence expressed in dendritic cells. In one embodiment, the siRNA inhibits expression of a target sequence expressed in myeloid cells. In one embodiment, the siRNA inhibits expression of a target sequence expressed in macrophages.

In another embodiment, the siRNA inhibits the expression of a transcription factor (e.g., FoxP3, RORc, T-bet, RoRγt, STAT3, AhR, NFkB) in the immune cell (e.g., T cells, B cells, NK cells, dendritic cells, myeloid cells, macrophages). In one embodiment, the siRNA inhibits the expression of a cytoplasmic protein (e.g., Mcl-1, HDAC10 histone deacetylase, asparaginyl endopeptidase (AEP), SOCS1, SOCS2, PPARg, GILZ, AMKa1, AMKa2, SHP-1, SHP-2, CAMKK2, IDO1, IDO2, TDO) in the immune cell (e.g., T cells, B cells, NK cells, dendritic cells, myeloid cells, macrophages). In another embodiment, the siRNA inhibits the expression of a transmembrane protein (e.g., cell surface receptors, such as antibodies, T cell receptors, immune checkpoint inhibitors) in the immune cell (e.g., T cells, B cells, NK cells, dendritic cells, myeloid cells, macrophages). In another embodiment, the siRNA inhibits the expression of a secreted protein (e.g., cytokines, chemokines) in the immune cell (e.g., T cells, B cells, NK cells, dendritic cells, myeloid cells, macrophages). In another embodiment, the siRNA inhibits the expression of an intracellular signaling protein in the immune cell (e.g., T cells, B cells, NK cells, dendritic cells, myeloid cells, macrophages). In another embodiment, the siRNA inhibits the expression of an enzyme (e.g., AMPKa1, AMPKa2, HDAC10, AEP, SHP-1, SHP-2, CAMKK2, IDO1, IDO2, TDO) in the immune cell (e.g., T cells, B cells, NK cells, dendritic cells, myeloid cells, macrophages).

MicroRNAs

MicroRNAs (miRNAs) are small non-coding RNA molecules (typically containing about 22 nucleotides) that function in RNA silencing and post-transcriptional regulation of gene expression. miRNAs inhibit gene expression via base-pairing with complementary sequences within mRNA molecules, leading to cleavage of the mRNA, destabilization of the mRNA through shortening of its polyA tail and/or less efficient translation of the mRNA into protein by ribosomes. With respect to mRNA cleavage, it has been demonstrated that given complete complementarity between the miRNA and the target mRNA sequence, the protein Ago2 can cleave the mRNA, leading to direct mRNA degradation. miRNAs and their function have been described in the art (see e.g., Ambros (2004) Nature 431:350-355; Bartel (2004) Cell 116:281-297; Bartel (2009) Cell 136:215-233; Fabian et al. (2010) Ann. Rev. Biochem. 79:351-379).

Accordingly, in one embodiment, the RNA interference agent associated with/encapsulated by the lipid-based composition, e.g., LNP, is a miRNA. In one embodiment, the miRNA inhibits expression of a target sequence expressed in immune cells. In one embodiment, the miRNA inhibits expression of a target sequence expressed in lymphoid cells. In one embodiment, the miRNA inhibits expression of a target sequence expressed in T cells. In one embodiment, the miRNA inhibits expression of a target sequence expressed in B cells. In one embodiment, the miRNA inhibits expression of a target sequence expressed in dendritic cells. In one embodiment, the miRNA inhibits expression of a target sequence expressed in myeloid cells. In one embodiment, the miRNA inhibits expression of a target sequence expressed in macrophages.

In another embodiment, the miRNA inhibits the expression of a transcription factor (e.g., FoxP3, RORc, T-bet, RoRγt, STAT3, AhR, NFkB) in the immune cell (e.g., T cells, B cells, NK cells, dendritic cells, myeloid cells, macrophages). In one embodiment, the miRNA inhibits the expression of a cytoplasmic protein (e.g., Mcl-1, HDAC10 histone deacetylase, asparaginyl endopeptidase (AEP), SOCS1, SOCS2, PPARg, GILZ, AMKa1, AMKa2, SHP-1, SHP-2, CAMKK2, IDO1, IDO2, TDO) in the immune cell (e.g., T cells, B cells, NK cells, dendritic cells, myeloid cells, macrophages). In another embodiment, the miRNA inhibits the expression of a transmembrane protein (e.g., cell surface receptors, such as antibodies, T cell receptors, immune checkpoint inhibitors) in the immune cell (e.g., T cells, B cells, NK cells, dendritic cells, myeloid cells, macrophages). In another embodiment, the miRNA inhibits the expression of a secreted protein (e.g., cytokines, chemokines) in the immune cell (e.g., T cells, B cells, NK cells, dendritic cells, myeloid cells, macrophages). In another embodiment, the miRNA inhibits the expression of an intracellular signaling protein in the immune cell (e.g., T cells, B cells, NK cells, dendritic cells, myeloid cells, macrophages). In another embodiment, the miRNA inhibits the expression of an enzyme (e.g., AMPKa1, AMPKa2, HDAC10, AEP, SHP-1, SHP-2, CAMKK2, IDO1, IDO2, TDO) in the immune cell (e.g., T cells, B cells, NK cells, dendritic cells, myeloid cells, macrophages).

For modulation of immune cell activity and/or modulation of immune responses, non-limiting examples of suitable miRNAs include Let-7d-5p, miR-7, miR-10a, miR-10b, miR-15, miR-18a, miR-20a, miR-20b, miR-21, miR-26a, miR-34a, miR-96, miR-99a, miR-100, miR-124, miR-125a, miR-126, miR-142-3p, miR-146, miR-150, miR-155, miR-181a and miR-210.

Exemplary Target mRNAs

The mRNA to be targeted by the RNA interference agent (e.g., targeted for knock down by an siRNA) can be chosen based on the desired outcome. Given that the LNPs of the invention have now been found to target immune cells, one of ordinary skill in the art can deliver various art-recognized RNA interference agents (e.g., siRNAs) to immune cells to the activity of various immune cells to thereby enhance or reduce immune responses.

For example, in one embodiment, the immune cell to which the LNP is delivered is one that naturally stimulates an immune response, such as T helper cells (e.g., Th17 cells), T effector cells (e.g., CTLs), B cells, NK cells, dendritic cells, macrophages. To downmodulate an immune response, an mRNA can be targeted within the cell that naturally stimulates the differentiation, activity and/or functional effects of the cell. Thus, knock down of such a target mRNA results in inhibition of the differentiation, activity and/or functional effects of the cell, thereby inhibiting immune responses. A non-limiting example of this type of approach is the delivery into Th17 cells of an RNA interference agent (e.g., siRNA) that targets a RAR-related orphan nuclear receptor (ROR) transcription factor (see Example 6). ROR transcription factors (including RORc, RORγt, RORα) have been demonstrated to play a significant role in the differentiation of Th17 cells through regulating the expression of various genes in the cells (see e.g., Huh, J. R and Littman, D. R. (2012) Eur. J. Immunol. 42:2232-2237; Castro, G. et al. (2017) PLoSOne 12(8):e01801868). Moreover, Th17 cells are known to express several pro-inflammatory cytokines and the actions of these cells have been linked to multiple human autoimmune disease. Thus, by targeting ROR in Th17 cells, the activity of the cells can be downmodulated to thereby downmodulate an immune response, for example in a subject with an autoimmune disorder. Similarly, another example of this type of approach is the delivery into Th17 cells of an RNA interference agent (e.g., siRNA) that targets mRNA encoding the cytokine IL-17a (see Example 6). IL-17a is a pro-inflammatory cytokine expressed by Th17 cells and thus by targeting IL-17a in these cells, the proinflammatory activity of the cells can be downmodulated to thereby downmodulate an immune response, for example in a subject with an autoimmune disorder. Additional proinflammatory cytokines produced by immune cells, such as T helper cells or macrophages, that are exemplary mRNA targets for an RNA interference agent (siRNA) of the disclosure include IL-1 (e.g., IL-1β), IL-6, IL-12, IL-18, IFN-γ, TNF-α and GM-CSF.

In another embodiment, the immune cell to which the LNP is delivered is one that naturally stimulates an immune response, such as T helper cells (e.g., Th17 cells), T effector cells (e.g., CTLs), B cells, NK cells, dendritic cells, macrophages. To (further) stimulate an immune response, an mRNA can be targeted within the cell that naturally inhibits the differentiation, activity and/or functional effects of the cell. Thus, knock down of such a (negative regulator) target mRNA results in promotion of the differentiation, activity and/or functional effects of the cell, thereby stimulating immune responses. A non-limiting example of this type of approach is the delivery into T cells of an RNA interference agent (e.g., siRNA) that targets mRNA encoding an immune checkpoint molecule in the T cell (such as PD-1, PD-L1, PD-L2, CTLA-4). Such immune checkpoint molecules serve to downregulate the activity of the T cell. Thus, by knocking down the activity of such (negative regulator) target mRNA, T cell activity is maintained or upregulated, thereby stimulating an immune response.

In another embodiment, the immune cell to which the LNP is delivered is one that naturally inhibits an immune response, such as Treg cells or Breg cells. To stimulate an immune response, an mRNA can be targeted within the cell that naturally stimulates the differentiation, activity and/or functional effects of these inhibitory cell. Thus, knock down of such a target mRNA results in inhibition of the differentiation, activity and/or functional effects of the inhibitory cell, thereby stimulating immune responses. A non-limiting example of this type of approach is the delivery into Treg cells of an RNA interference agent (e.g., siRNA) that targets the Foxp3 transcription factor (see Examples 1-5). Foxp3 has been demonstrated to play a significant role in the differentiation and function of Treg cells through regulating the expression of various genes in the cells (see e.g., Zhiyuan, L. et al. (2015) Cell. Mol. Immunol. 12:558-565; Bluestone, J. A. (2017) J. Immunol. 198:979-980). Thus, by targeting Foxp3 in Treg cells, the activity of these inhibitory cells can be downmodulated to thereby stimulate an immune response, for example in a subject with cancer or an infectious disease. Similarly, another example of this type of approach is the delivery into Breg cells of an RNA interference agent (e.g., siRNA) that targets mRNA encoding the cytokine IL-10. Breg cells, which suppress immune responses, mediate their effects at least in part through the cytokine IL-10. Thus, by targeting IL-10 in these inhibitory B cells, the inhibitory effect of these cells can be downmodulated to thereby stimulate an immune response, for example in a subject with cancer.

In yet another embodiment, the immune cell to which the LNP is delivered is one that naturally downmodulates an immune response, such as Treg cells or Breg cells. To (further) inhibit an immune response, an mRNA can be targeted within such cells that naturally inhibits the differentiation, activity and/or functional effects of these cells. Thus, knock down of such a (negative regulator) target mRNA results in promotion of the differentiation, activity and/or functional effects of these inhibitory cells, thereby inhibiting immune responses.

In certain embodiments, the immune cell to which the LNP is delivered is a Treg cell and the RNA interference agent (e.g., siRNA) inhibits (i.e., decreases) Treg suppressive function. Non-limiting examples include RNA interference agents (e.g., siRNA) that target Foxp3 or proteins that interact with Foxp3, such as IRF4, ablation of which has been shown to inhibit the suppressive function of Tregs (see e.g., Zheng, Y. et al. (2009) Nature 458:351-356), as well as RNA interference agents (e.g., siRNA) that target estrogen receptor 1, ablation of which has also been shown to inhibit the suppressive function of Tregs (see e.g., McKarns, S. (2015) J. Immunol. 194 (Suppl. 1):184.21).

In certain embodiments, the immune cell to which the LNP is delivered is a Treg cell and the RNA interference agent (e.g., siRNA) augments (i.e., increases) Treg suppressive function. Non-limiting examples include RNA interference agents (e.g., siRNA) that target histone deacetylase 6, 10 or 11 (HDAC6, HDAC10 or HDAC11) or asparaginyl endopeptidase (AEP).

In certain embodiments, the immune cell to which the LNP is delivered is a Treg cell and the RNA interference agent (e.g., siRNA) targets an miRNA, such as miR-146b. Knockdown of miR-146b in Treg has been shown to enhance Treg survival, proliferation and suppressive function (see e.g., Lu, Y. et al. (2016) Blood 128:1424-1435). Accordingly, an RNA interference agent (e.g., siRNA) that knocks down miR-146b can be used to enhance Treg suppressive function, whereas an RNA interference agent (e.g., siRNA) that knocks down anti-miR-146b can be used to inhibit Treg suppressive function.

In certain embodiments, the immune cell to which the LNP is delivered is a Teff cell and the RNA interference agent (e.g., siRNA) causes Teff dysregulation (e.g., suppression of Teff responses, skewing of Th17 cells to Treg cells). Non-limiting examples include RNA interference agents (e.g., siRNA) that target Kv1.3 potassium channels, KCA3.1 calcium-activated potassium channel, KCNN4 potassium calcium-activated channel, Tbet transcription factor, RORγt transcription factor and IL-17a.

In certain embodiments, the immune cell to which the LNP is delivered is a myeloid cell and the RNA interference agent (e.g., siRNA) modulates myeloid cell activity. Non-limiting examples include RNA interference agents (e.g., siRNA) that target anti-miR-33, miR-99a, Camk4 and miR-10b.

In certain embodiments, the RNA interference agent (e.g., siRNA) encapsulated by the LNP targets a component of a mammalian target of rapamycin complex (mTORC), such as a component of mTORC1 or mTORC2. For example, in one embodiment, the RNA interference agent (e.g., siRNA) encapsulated by the LNP targets Raptor, a component of mTORC1. In another embodiment, the RNA interference agent (e.g., siRNA) encapsulated by the LNP targets Rictor, a component of mTORC2.

Additional specific proteins (e.g., cytokines, chemokines, costimulatory molecules, recruitment factors, transcription factors, effector molecules) that can be inhibited by the RNA interference agent (e.g., siRNA) to thereby modulate immune responses (upregulation or downregulation) are described in detail in the following subsection.

Soluble Targets

In one embodiment, the RNA interference agent associated with/encapsulated by the lipid-based composition, e.g., LNP, modulates the activity of a naturally-occurring soluble target by modulating the expression of the soluble target in an immune cell (e.g., T cell, B cell, NK cell, dendritic cell, myeloid cell, macrophage). In one embodiment, the cell is a lymphocyte. Non-limiting examples of naturally-occurring soluble targets include cytokines and chemokines. Suitable cytokines and chemokines for particular uses in stimulating or inhibiting immune responses are described further below.

In one embodiment, the method of using the lipid-based composition, e.g. LNP, is used to stimulate (upregulate, enhance) the activation or activity of an immune cell, for example in situations where stimulation of an immune response is desirable, such as in cancer therapy or treatment of an infectious disease (e.g., a viral, bacterial, fungal, protozoal or parasitic infection). In another embodiment, the method of using the lipid-based composition, e.g. LNP, is used to inhibit (downregulate, reduce) the activation or activity of an immune cell, for example in situations where inhibition of an immune response is desirable, such as in autoimmune diseases, allergies and transplantation.

In one embodiment of modulating the activation or activity of an immune cell, the mRNA targeted by the RNA interference agent (e.g., siRNA) is a cytokine such that the levels of expression of the cytokine in the immune cell are knocked down. Cytokines are mediators of intracellular signaling that regulate the immune system. Non-limiting examples of cytokines that can stimulate immune cell activation or activity include IL-1 (pro-inflammatory cytokine), IL-2 (T cell growth factor that promotes T cell differentiation), IL-3 (stimulates proliferentiation of myeloid lineage cells), IL-4 (stimulates B and T cell proliferation and B cell differentiation), IL-5 (stimulates B cell growth), IL-6 (pro-inflammatory), IL-7 (stimulates differentiation of lymphoid lineage cells), IL-12 (differentiation of naïve T cells to Th1 cells), IL-13 (stimulation of activated B and T cell proliferation and B cell differentiation), IL-15 (regulation of activation and proliferation of T cells and NK cells), IL-17 (proinflammatory and induces chemokines), IL-18 (proinflammatory and promotes IFN release), IL-21 (proinflammatory and regulates NK and CTL proliferation), IL-23 (proinflammatory), TNFα (stimulates systemic inflammation and inhibits tumorigenesis and viral replication), TNFβ (regulates development of secondary lymphoid organs), IFNα involved in innate immunity to viral infection), IFNβ (involved in innate immunity to viral infection), IFNγ (involved in innate and adaptive immunity to viruses and other infectious agents), GM-CSF (stimulates white blood cell production and enhances anti-tumor T cells), G-CSF (stimulates white blood cell production), and a combination thereof.

In one embodiment, the cytokine is a pro-inflammatory cytokine, non-limiting examples of which include IL-1, IL-6, IL-17, IL-18, IL-23, TNFα, IFN-α, IFN-β and IFN-γ. A pro-inflammatory cytokine can be used in situations in which stimulation of an inflammatory response is desired, for example to increase anti-tumor immunity in cancer therapy or in viral infections. In one embodiment, the cytokine promotes T cell activation. Non-limiting examples of cytokines that promote T cell activation or differentiation include IL-2, IL-4, IL-12, IL-13, IL-15, and IFN-α. In one embodiment, the cytokine promotes Th2 responses. Non-limiting examples of cytokines that promote Th2 responses include IL-4 and IL-10. In one embodiment, the cytokine promotes B cell activation. Non-limiting examples of cytokines that promote B cell activation include IL-4, IL-5, IL-6, IL-10, IL-13 and IFN (e.g., IFN-α, IFN-β and IFN-γ).

In one embodiment of modulating the activation or activity of an immune cell, the mRNA targeted by the RNA interference agent (e.g., siRNA) is a chemokine or chemokine receptor such that the levels of expression of the chemokine or chemokine receptor in the immune cell are knocked down. Chemokines have been demonstrated to be substances that control the trafficking of inflammatory cells (including granulocytes and monocytes/macrophages), as well as regulating the movement of a wide variety of immune cells (including lymphocytes, natural killer cells and dendritic cells). Thus, chemokines are involved both in regulating inflammatory responses and immune responses. Moreover, chemokines have been shown to have effects on the proliferative and invasive properties of cancer cells (for a review of chemokines, see e.g., Mukaida, N. et al. (2014) Mediators of Inflammation. Article ID 170381, pg. 1-15). In one embodiment, the chemokine or chemokine receptor acts on regulatory T cells, non-limiting examples of which include CCL22, CCL28, CCR4 and CCR10. In another embodiment, the chemokine or chemokine receptor acts on cytotoxic T cells, non-limiting examples of which include CXCL9, CXCL10, CXCL11 and CXCR3. In another embodiment, the chemokine or chemokine receptor acts on natural killer cells, non-limiting examples of which include CXCL9, CXCL10, CXCL11, CCL3, CCL4, CCL5, CCL2, CCL8, CCL12, CCL13, CCL19, CCL21, CX3CL1, CXCR3, CCR1, CCR5, CCR2 and CX3CR1. In another embodiment, the chemokine or chemokine receptor acts on immature dendritic cells, non-limiting examples of which include CCL3, CCL4, CCL5, CCL2, CCL7, CCL8, CCL22, CCL1, CCL17, CXCL12, CCR1, CCR2, CCR4, CCR5, CCR6, CCR8 and CXCR4. In another embodiment, the chemokine or chemokine receptor acts on mature dendritic cells, non-limiting examples of which include CCL19, CCL21, CXCL12, CCR7 and CXCR4. In another embodiment, the chemokine or chemokine receptor acts on tumor-associated macrophages, non-limiting examples of which include CCL2, CCL7, CCL8, CCL3, CCL4, CCL5, CXCL12, CCR2, CCR5 and CXCR4.

In one embodiment of modulating the activation or activity of an immune cell, the mRNA targeted by the RNA interference agent (e.g., siRNA) encodes a recruitment factor. As used herein a “recruitment factor” refers to any protein that promotes recruitment of an immune cell to a desired location (e.g., to a tumor site or an inflammatory site). For example, certain chemokines, chemokine receptors and cytokines have been shown to be involved in the recruitment of lymphocytes (see e.g., Oelkrug, C. and Ramage, J. M. (2014) Clin. Exp. Immunol. 178:1-8). Non-limiting examples of recruitment factors include CXCR3, CXCR5, CCR5, CCL5, CXCL10, CXCL12, CXCL16 and IFN-γ.

Intracellular Targets

In one embodiment, the RNA interference agent (e.g., siRNA) associated with/encapsulated by the lipid-based composition, e.g., LNP, modulates the activity of a naturally-occurring intracellular target by modulating the expression of the intracellular target in an immune cell (e.g., T cell, B cell, NK cell, dendritic cell, myeloid cell, macrophage). In one embodiment, the cell is a lymphoid cell. Non-limiting examples of naturally-occurring intracellular targets include transcription factors and cell signaling cascade molecules, including enzymes. Suitable transcription factors and intracellular signaling cascade molecules for particular uses in stimulating or inhibiting immune responses are described further below.

In one embodiment of modulating the activation or activity of an immune cell, the protein target is a transcription factor. As used herein, a “transcription factor” refers to a DNA-binding protein that regulates the transcription of a gene. In one embodiment, the protein is a transcription factor that increases or polarizes an immune response. In one embodiment, the protein is a transcription factor that stimulates a Type I IFN response. In another embodiment, the protein is a transcription factor that stimulates an NFKB-mediated proinflammatory response. Non-limiting examples of transcription factors include Interferon Regulatory Factors (IRFs, including IRF-1, IRF-3, IRF-5, IRF-7, IRF-8 and IRF-9), CREB, RORc, RORγ, RORγt, RORα, SOCS, NFκB, FoxP3, T-bet, STAT3 and AhR.

In one embodiment of modulating the activation or activity of an immune cell, the protein target is an intracellular adaptor protein. In one embodiment, the intracellular adaptor protein stimulates a Type I IFN response. In another embodiment, the intracellular adaptor protein stimulates an NFκB-mediated proinflammatory response. Non-limiting examples of intracellular adaptor proteins that stimulate a Type I IFN response and/or stimulate and NFκB-mediated proinflammatory response include STING, MAVS and MyD88.

In one embodiment of modulating the activation or activity of an immune cell, the protein target is an intracellular signaling protein. In one embodiment, the protein is an intracellular signaling protein of a TLR signaling pathway. In one embodiment, the intracellular signalling protein stimulates a Type I IFN response. In another embodiment, the intracellular signalling protein stimulates an NFκB-mediated proinflammatory response. Non-limiting examples of intracellular signalling proteins that stimulate a Type I IFN response and/or stimulate an NFκB-mediated proinflammatory response include MyD88, IRAK 1, IRAK2, IRAK4, TRAF3, TRAF6, TAK1, TAB2, TAB3, TAK-TAB1, MKK3, MKK4, MKK6, MKK7, IKKα, IKKβ, TRAM, TRIF, RIPK1, and TBK1.

Other non-limiting examples of intracellular signaling molecules for up- or down-regulation of immune responses include Mcl-1, AMPKa1, AMPKa2, GILZ, PPARg, HDAC10, AEP, SHP-1, SHP-2, CAMKK2 IDO1, IDO2 and TDO.

In one embodiment of modulating the activation or activity of an immune cell, the mRNA encodes a transcription factor, e.g., a tolerogenic transcription factor that promotes tolerance, such as RelA, Runx1, Runx3 and FoxP3.

Membrane Bound/Transmembrane Targets

In one embodiment, the RNA interference agent (e.g., siRNA) associated with/encapsulated by the lipid-based composition, e.g., LNP, modulates the activity of a naturally-occurring membrane-bound/transmembrane target by modulating the expression of the membrane-bound/transmembrane target in an immune cell (e.g., T cell, B cell, NK cell, dendritic cell, myeloid cell, macrophage). Non-limiting examples of naturally-occurring membrane-bound/transmembrane targets include costimulatory molecules, immune checkpoint molecules, homing signals and HLA molecules. Suitable membrane-bound/transmembrane targets for particular uses in stimulating or inhibiting immune responses are described further below.

In one embodiment of modulating the activation or activity of an immune cell, the protein target is a costimulatory factor that upregulates immune response or is an antagonist of a costimulatory factor that downregulates immune responses. Non-limiting examples of costimulatory factors that upregulate immune responses include CD28, CD80, CD86, ICOS, ICOSL, OX40, OX40L, CD40, CD40L, GITR, GITRL, CD137 and CD137L. Non-limiting examples of costimulatory molecules that downregulate immune response include PD-1, PD-L1, PD-L2 and CTLA-4. In one embodiment of modulating the activation or activity of an immune cell, the protein target is an immune checkpoint protein that down-regulates immune cells (e.g., T cells), non-limiting examples of which include CTLA-4, PD-1 and PD-L1 and PD-L2.

In one embodiment, the membrane-bound/transmembrane protein target is a homing signal.

In one embodiment, the membrane-bound/transmembrane protein target is an HLA molecule, such as an HLA-G. The non-classical HLA class I molecule HLA-G is a potent inhibitory molecule that protects the cells that express it from cytolysis. This function has been reported as being crucial for the protection of the fetal cytotrophoblasts from destruction by the maternal immune system, for the protection of allografts against cytolysis by the recipient's immune system and for the protection of tumors against anti-tumor immunity. Accordingly, RNA interference agents (e.g., siRNA) that downregulate HLA-G, can be used to promote immune-mediated cytolysis, such as in tumor-bearing subjects to stimulate anti-tumor immunity.

### Synthesis and Modification of RNA Interference Agents

RNA interference agents such as siRNAs and miRNAs can be prepared by methods well established in the art. Currently there are five methods that have been used to generate RNA interference agents: chemical synthesis, in vitro transcription, digestion of long double-stranded RNA (dsRNA) by an RNase III family enzyme (e.g., Dicer, RNase III), expression in cells from an expression plasmid or viral vector and expression in cells from a PCR-derived expression cassette. Moreover, custom design and synthesis of RNA interference agents is commercially available (e.g., Dharmacon, ThermoFisher Scientific).

In one embodiment, an RNA interference agent (e.g., siRNA) of the disclosure is comprised of unmodified nucleobases, nucleosides or nucleotides. In another embodiment, an RNA interference agent (e.g., siRNA) of the disclosure comprises one or more modified nucleobases, nucleosides or nucleotides. In some embodiments, modified RNA interference agents (e.g., siRNA) may have useful properties, including enhanced stability, intracellular retention and/or the lack of a substantial induction of the innate immune response of a cell into which the agent is introduced, as compared to a reference unmodified agent. Therefore, use of a modified RNA interference agent may enhance the efficiency of function of the agent and/or intracellular retention of the agent, as well as reduce immunogenicity of the agent.

In some embodiments, an RNA interference agent (e.g., siRNA) includes one or more (e.g., 1, 2, 3 or 4) different modified nucleobases, nucleosides, or nucleotides. In some embodiments, the agent includes one or more (e.g., 1, 2, 3, 4, 5, 6, 7, 8, 9, 10 or more) different modified nucleobases, nucleosides, or nucleotides. In some embodiments, the modified agent may have reduced degradation in a cell into which the agent is introduced, relative to a corresponding unmodified agent.

For example, in one embodiment, the RNA interference agent (e.g., siRNA) comprises 2′-O-methylation of at least one nucleoside (i.e., a methyl group is added to the 2′hydroxyl of the ribose moiety of at least one nucleoside in the agent). A modified RNA interference agent can comprise at least one 2′-O-methyl-adenosine, at least one 2′-O-methyl-guanosine, at least one 2′-O-methyl-uruacil, at least one 2′-O-methyl-cytosine, or any combination thereof.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (im5U), 1-taurinomethyl-pseudouridine, 5-taurinomethyl-2-thio-uridine(im5s2U), 1-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (Wm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include a-thio-adenosine, 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include a-thio-guanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (mlIm), 2′-O-ribosylguanosine (phosphate) (Gr(p)), 1-thio-guanosine, 06-methyl-guanosine, 2′-F-ara-guanosine, and 2′-F-guanosine.

In some embodiments, an RNA interference agent (e.g., siRNA) of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an RNA interference agent (e.g., siRNA) of the disclosure includes a combination of one or more of the aforementioned modified nucleobases (e.g., a combination of 2, 3 or 4 of the aforementioned modified nucleobases.) In one embodiment, the modified nucleobase is N1-methylpseudouridine (m1ψ) and the RNA interference agent (e.g., siRNA) of the disclosure is fully modified with N1-methylpseudouridine (m1ψ). In some embodiments, N1-methylpseudouridine (m1ψ) represents from 75-100% of the uracils in the agent. In some embodiments, N1-methylpseudouridine (m1ψ) represents 100% of the uracils in the agent.

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an RNA interference agent (e.g., siRNA) of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an RNA interference agent (e.g., siRNA) of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an RNA interference agent (e.g., siRNA) of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m1ψ), 5-methoxy-uridine (mo5U), 5-methyl-cytidine (m5C), pseudouridine (ψ), α-thio-guanosine, or α-thio-adenosine. In some embodiments, an RNA interference agent (e.g., siRNA) of the disclosure includes a combination of one or more of the aforementioned modified nucleobases (e.g., a combination of 2, 3 or 4 of the aforementioned modified nucleobases.)

In some embodiments, the agent comprises pseudouridine (ψ). In some embodiments, the agent comprises pseudouridine (ψ) and 5-methyl-cytidine (m5C). In some embodiments, the agent comprises 1-methyl-pseudouridine (m1ψ). In some embodiments, the agent comprises 1-methyl-pseudouridine (m1ψ) and 5-methyl-cytidine (m5C). In some embodiments, the agent comprises 2-thiouridine (s2U). In some embodiments, the agent comprises 2-thiouridine and 5-methyl-cytidine (m5C). In some embodiments, the agent comprises 5-methoxy-uridine (mo5U). In some embodiments, the agent comprises 5-methoxy-uridine (mo5U) and 5-methyl-cytidine (m5C). In some embodiments, the agent comprises 2′-O-methyl uridine. In some embodiments, the agent comprises 2′-O-methyl uridine and 5-methyl-cytidine (m5C). In some embodiments, the agent comprises comprises N6-methyl-adenosine (m6A). In some embodiments, the agent comprises N6-methyl-adenosine (m6A) and 5-methyl-cytidine (m5C).

In certain embodiments, an RNA interference agent (e.g., siRNA) of the disclosure is uniformly modified (i.e., fully modified, modified through-out the entire sequence) for a particular modification. For example, an agent can be uniformly modified with N1-methylpseudouridine (m1ψ) or 5-methyl-cytidine (m5C), meaning that all uridines or all cytosine nucleosides in the agent sequence are replaced with N1-methylpseudouridine (m1ψ) or 5-methyl-cytidine (m5C). Similarly, agents of the disclosure can be uniformly modified for any type of nucleoside residue present in the sequence by replacement with a modified residue such as those set forth above.

The RNA interference agents (e.g., siRNAs) of the disclosure can include a combination of modifications to the sugar, the nucleobase, and/or the internucleoside linkage. These combinations can include any one or more modifications described herein.

In certain embodiments, the modified nucleosides may be partially or completely substituted for the natural nucleotides of the agents of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

### Lipid Nanoparticles

An RNA interference agent of the disclosure, e.g. siRNA, is encapsulated in a lipid nanoparticle to facilitate delivery of the polynucleotide sequence into immune cells. Accordingly, in one set of embodiments, lipid nanoparticles (LNPs) are provided. Each of the LNPs described herein may be used as a formulation for siRNA described herein. In one embodiment, a lipid nanoparticle comprises lipids including an ionizable lipid, a sterol or other structural lipid, a non-cationic helper lipid or phospholipid, optionally a PEG lipid, and one or more polynucleotides, e.g., siRNAs.

In certain embodiments, the LNP includes an immune cell delivery potentiating lipid, which promotes delivery of the siRNA into immune cells. In one embodiment, the LNP comprises a phytosterol or a combination of a phytosterol and cholesterol. In one embodiment, the phytosterol is selected from the group consisting of β-sitosterol, stigmasterol, β-sitostanol, campesterol, brassicasterol, and combinations thereof. In one embodiment, the phytosterol is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

While not intending to be bound by any particular mechanism or theory, the enhanced delivery of an RNA interference agent to immune cells by the LNPs of the disclosure is believed to be due to the presence of an effective amount of an immune cell delivery potentiating lipid, e.g., a cholesterol analog or an amino lipid or combination thereof, that, when present in an LNP, may function by enhancing cellular association and/or uptake, internalization, intracellular trafficking and/or processing, and/or endosomal escape and/or may enhance recognition by and/or binding to immune cells, relative to an LNP lacking the immune cell delivery potentiating lipid. Furthermore, it was observed in in vitro experiments that serum was absolutely required for immune cell uptake/cell association of the LNP. Through depletion of various serum components, it was determined that complement component 1q (C1q) was involved in the uptake of the LNP by the immune cells. Accordingly, while not intending to be bound by any particular mechanism or theory, in one embodiment, an immune cell delivery potentiating lipid of the disclosure binds to C1q or promotes the binding of an LNP comprising such lipid to C1q. Thus, for in vitro use of the LNPs of the disclosure for delivery of a nucleic acid molecule to an immune cell, culture conditions that include C1q are used (e.g., use of culture media that includes serum or addition of exogenous C1q to serum-free media). For in vivo use of the LNPs of the disclosure, the requirement for C1q is supplied by endogenous C1q.

Immune cell delivery LNPs comprise an (i) ionizable lipid; (ii) sterol or other structural lipid; (iii) optionally, a non-cationic helper lipid or phospholipid; (iv) optionally, a PEG lipid and (v) an RNA interference agent (e.g., siRNA) encapsulated in and/or associated with the LNP, wherein one or more of (i) the ionizable lipid or (ii) the structural lipid or sterol in an immune cell delivery LNPs comprises an effective amount of an immune cell delivery potentiating lipid.

In another embodiment, an immune cell delivery lipid nanoparticle of the disclosure comprises:

(i) an ionizable lipid;

(ii) a sterol or other structural lipid;

(iii) a non-cationic helper lipid or phospholipid;

(iv) an RNA interference agent (e.g., siRNA); and

(v) optionally, a PEG-lipid

wherein one or more of (i) the ionizable lipid or (ii) the sterol or other structural lipid comprises an immune cell delivery potentiating lipid in an amount effective to enhance delivery of the lipid nanoparticle to an immune cell. In one embodiment, enhanced delivery is relative to a lipid nanoparticle lacking the immune cell delivery potentiating lipid. In another embodiment, the enhanced delivery is relative to a suitable control.

In another embodiment, an immune cell delivery lipid nanoparticle of the disclosure comprises:

(i) an ionizable lipid;

(ii) a sterol or other structural lipid;

(iii) a non-cationic helper lipid or phospholipid;

(iv) an RNA interference agent (e.g., siRNA), and

(v) optionally, a PEG-lipid

wherein one or more of (i) the ionizable lipid or (ii) the sterol or other structural lipid or (iii) the non-cationic helper lipid or phospholipid or (v) the PEG lipid is a C1q binding lipid that binds to C1q or promotes (e.g., increases, stimulates, enhances) the binding of the LNP to C1q, as compared to a control LNP lacking the C1q binding lipid.

In another embodiment, an immune cell delivery lipid nanoparticle of the disclosure comprises:

(i) an ionizable lipid;

(ii) a sterol or other structural lipid;

(iii) a non-cationic helper lipid or phospholipid;

(iv) an RNA interference agent (e.g., siRNA), and

(v) optionally, a PEG-lipid

wherein one or more of (i) the ionizable lipid or (ii) the sterol or other structural lipid binds to C1q or promotes (e.g., increases, stimulates, enhances) the binding of the LNP to C1q, as compared to a control LNP (e.g., an LNP lacking (i) the ionizable lipid or (ii) the sterol or other structural lipid).

In another aspect, the disclosure provides a method of screening for an immune cell delivery lipid, the method comprising contacting a test LNP comprising a test immune cell delivery lipid with C1q, and measuring binding to C1q, wherein a test immune cell delivery lipid is selected as an immune cell delivery lipid when it binds to C1q or promotes (e.g., increases, stimulates, enhances) the binding of the LNP comprising it to C1q.

### Lipid Content of LNPs

As set forth above, with respect to lipids, immune cell delivery LNPs comprise an (i) ionizable lipid; (ii) sterol or other structural lipid; (iii) a non-cationic helper lipid or phospholipid; a (iv) PEG lipid, wherein one or more of (i) the ionizable lipid or (ii) the structural lipid or sterol in an immune cell delivery LNPs comprises an effective amount of an immune cell delivery potentiating lipid. These categories of lipids are set forth in more detail below.

(i) Ionizable Lipids

The lipid nanoparticles of the present disclosure include one or more ionizable lipids. In certain embodiments, the ionizable lipids of the disclosure comprise a central amine moiety and at least one biodegradable group. The ionizable lipids described herein may be advantageously used in lipid nanoparticles of the disclosure for the delivery of nucleic acid molecules to mammalian cells or organs. The structures of ionizable lipids set forth below include the prefix I to distinguish them from other lipids of the invention.

In a first aspect of the invention, the compounds described herein are of Formula (I I):

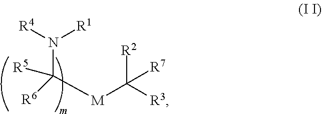

or their N-oxides, or salts or isomers thereof, wherein:

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected

from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl;

each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13; and wherein when R⁴ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

Another aspect the disclosure relates to compounds of Formula (III), also referred to as Formula (I III):

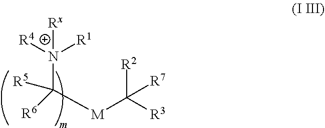

### or its N-oxide,

or a salt or isomer thereof, wherein

or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;

R^{x} is selected from the group consisting of C₁₋₆ alkyl, C₂₋₆ alkenyl, —(CH₂)ᵥOH, and —(CH₂)ᵥN(R)₂,

wherein v is selected from 1, 2, 3, 4, 5, and 6;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

R⁸ is selected from the group consisting of C₃₋₆ carbocycle and heterocycle;

R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

R¹⁰ is selected from the group consisting of H, OH, C₁₋₃ alkyl, and C₂₋₃ alkenyl;

each R is independently selected from the group consisting of C₁₋₃ alkyl, C₂₋₃ alkenyl, (CH₂)_{q}OR*, and H,

and each q is independently selected from 1, 2, and 3;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In certain embodiments, a subset of compounds of Formula (I), also referred to as Formula (I I), includes those of Formula

(IA), also referred to as Formula (I IA):

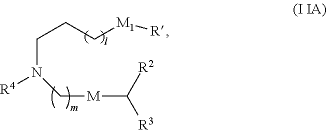

or its N-oxide, or a salt or isomer thereof, wherein 1 is selected from 1, 2, 3, 4, and 5; m is selected from 5, 6, 7, 8, and 9; M1 is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group,; and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB), also referred to as Formula (IIB):

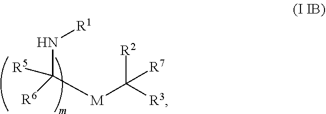

or its N-oxide, or a salt or isomer thereof in which all variables are as defined herein. For example, m is selected from 5, 6, 7, 8, and 9; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. In certain embodiments, a subset of compounds of Formula (I) includes those of Formula (I I), also referred to as Formula (I II):

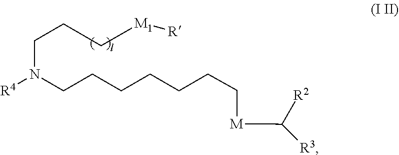

or its N-oxide, or a salt or isomer thereof, wherein 1 is selected from 1, 2, 3, 4, and 5; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl.

Another aspect of the disclosure relates to compounds of Formula (IVI):

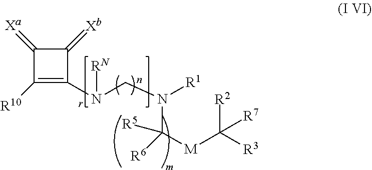

or its N-oxide, or a salt or isomer thereof, wherein

R¹ is selected from the group consisting of C₅₋₃₀ alkyl, C₅₋₂₀ alkenyl, —R*YR″, —YR″, and —R″M′R′;

R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;

each R⁵ is independently selected from the group consisting of OH, C₁₋₃ alkyl, C₂₋₃ alkenyl, and H;

each R⁶ is independently selected from the group consisting of OH, C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R⁷ is selected from the group consisting of C₁₋₃ alkyl, C₂₋₃ alkenyl, and H;

each R is independently selected from the group consisting of H, C₁₋₃ alkyl, and C₂₋₃ alkenyl;

R^{N} is H, or C₁₋₃ alkyl;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I;

X^{a} and X^{b} are each independently O or S;
R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, a carbocycle, a heterocycle, aryl and heteroaryl;
m is selected from 5, 6, 7, 8, 9, 10, 11, 12, and 13;
n is selected from 1, 2, 3, 4, 5, 6, 7, 8, 9, and 10;
r is 0 or 1;
t¹ is selected from 1, 2, 3, 4, and 5;
p¹ is selected from 1, 2, 3, 4, and 5;
q¹ is selected from 1, 2, 3, 4, and 5; and
s¹ is selected from 1, 2, 3, 4, and 5.

In one embodiment, a subset of compounds of Formula (VI), also referred to as Formula (I VI), includes those of Formula (VI-a), also referred to as Formula (I VI-a):

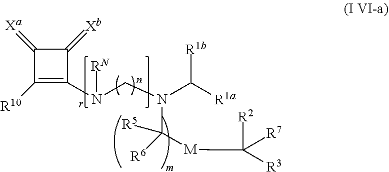

### or its N-oxide,

or a salt or isomer thereof, wherein

R^{1a} and R^{1b} are independently selected from the group consisting of C₁₋₁₄ alkyl and C₂₋₁₄ alkenyl; and

R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle.

In another embodiment, a subset of compounds of Formula (VI) includes those of Formula (VII), also referred to as Formula (I VII):

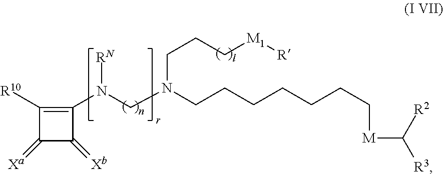

or its N-oxide, or a salt or isomer thereof, wherein

1 is selected from 1, 2, 3, 4, and 5;

M₁ is a bond or M′; and

R² and R³ are independently selected from the group consisting of H, C₁₋₁₄ alkyl, and C₂₋₁₄ alkenyl.

In another embodiment, a subset of compounds of Formula (I VI) includes those of Formula (I VIII):

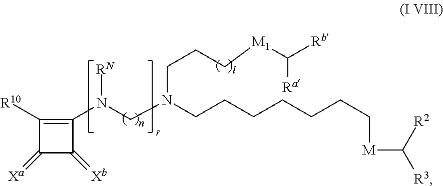

or its N-oxide, or a salt or isomer thereof, wherein

1 is selected from 1, 2, 3, 4, and 5;

M₁ is a bond or M′; and

R^{a′} and R^{b′} are independently selected from the group consisting of C₁₋₁₄ alkyl and C₂₋₁₄ alkenyl; and

R² and R³ are independently selected from the group consisting of C₁₋₁₄ alkyl, and C₂₋₁₄ alkenyl.

The compounds of any one of formula (I I), (I IA), (I VI), (I VI-a), (I VII) or (I VIII) include one or more of the following features when applicable.

In some embodiments, M₁ is M′.

In some embodiments, M and M′ are independently —C(O)O— or —OC(O)—.

In some embodiments, at least one of M and M′ is —C(O)O— or —OC(O)—.

In certain embodiments, at least one of M and M′ is —OC(O)—.

In certain embodiments, M is —OC(O)— and M′ is —C(O)O—. In some embodiments, M is —C(O)O— and M′ is —OC(O)—. In certain embodiments, M and M′ are each —OC(O)—. In some embodiments, M and M′ are each —C(O)O—.

In certain embodiments, at least one of M and M′ is —OC(O)-M″-C(O)O—.

In some embodiments, M and M′ are independently —S—S—.

In some embodiments, at least one of M and M′ is —S—S.

In some embodiments, one of M and M′ is —C(O)O— or —OC(O)— and the other is —S—S—.

For example, M is —C(O)O— or —OC(O)— and M′ is —S—S— or M′ is —C(O)O—, or —OC(O)— and M is —S—S—.

In some embodiments, one of M and M′ is —OC(O)-M″-C(O)O—, in which M″ is a bond, C₁₋₁₃ alkyl or C₂₋₁₃ alkenyl. In other embodiments, M″ is C₁₋₆ alkyl or C₂₋₆ alkenyl. In certain embodiments, M″ is C₁₋₄ alkyl or C₂₋₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, 1 is 1, 3, or 5.

In some embodiments, R⁴ is hydrogen.

In some embodiments, R⁴ is not hydrogen.

In some embodiments, R⁴ is unsubstituted methyl or —(CH₂)ₙQ, in which Q is OH, —NHC(S)N(R)₂, —NHC(O)N(R)₂, —N(R)C(O)R, or —N(R)S(O)₂R.

In some embodiments, Q is OH.

In some embodiments, Q is —NHC(S)N(R)₂.

In some embodiments, Q is —NHC(O)N(R)₂.

In some embodiments, Q is —N(R)C(O)R.

In some embodiments, Q is —N(R)S(O)₂R.

In some embodiments, Q is —O(CH₂)ₙN(R)₂.

In some embodiments, Q is —O(CH₂)ₙOR.

In some embodiments, Q is —N(R)R⁸.

In some embodiments, Q is —NHC(═NR⁹)N(R)₂.

In some embodiments, Q is —NHC(═CHR⁹)N(R)₂.

In some embodiments, Q is —OC(O)N(R)₂.

In some embodiments, Q is —N(R)C(O)OR.

In some embodiments, n is 2.

In some embodiments, n is 3.

In some embodiments, n is 4.

In some embodiments, M₁ is absent.

In some embodiments, at least one R⁵ is hydroxyl. For example, one R⁵ is hydroxyl.

In some embodiments, at least one R⁶ is hydroxyl. For example, one R⁶ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen.

In some embodiments, R^{x} is C₁₋₆ alkyl. In some embodiments, R^{x} is C₁₋₃ alkyl. For example, R^{x} is methyl. For example, R^{x} is ethyl. For example, R^{x} is propyl.

In some embodiments, R^{x} is —(CH₂)ᵥOH and, v is 1, 2 or 3. For example, R^{x} is methanoyl. For example, R^{x} is ethanoyl. For example, R^{x} is propanoyl.

In some embodiments, R^{x} is —(CH₂)ᵥN(R)₂, v is 1, 2 or 3 and each R is H or methyl. For example, R^{x} is methanamino, methylmethanamino, or dimethylmethanamino. For example, R^{x} is aminomethanyl, methylaminomethanyl, or dimethylaminomethanyl. For example, R^{x} is aminoethanyl, methylaminoethanyl, or dimethylaminoethanyl. For example, R^{x} is aminopropanyl, methylaminopropanyl, or dimethylaminopropanyl.

In some embodiments, R′ is C₁₋₁₈ alkyl, C₂₋₁₈ alkenyl, —R*YR″, or —YR″.

In some embodiments, R² and R³ are independently C₃₋₁₄ alkyl or C₃₋₁₄ alkenyl.

In some embodiments, R^{1b} is C₁₋₁₄ alkyl. In some embodiments, R^{1b} is C₂₋₁₄ alkyl. In some embodiments, R^{1b} is C₃₋₁₄ alkyl. In some embodiments, R^{1b} is C₁₋₈ alkyl. In some embodiments, R^{1b} is C₁₋₅ alkyl. In some embodiments, R^{1b} is C₁₋₃ alkyl. In some embodiments, R^{1b} is selected from C₁ alkyl, C₂ alkyl, C₃ alkyl, C₄ alkyl, and C₅ alkyl. For example, in some embodiments, R^{1b} is C₁ alkyl. For example, in some embodiments, R^{1b} is C₂ alkyl. For example, in some embodiments, R^{1b} is C₃ alkyl. For example, in some embodiments, R^{1b} is C₄ alkyl. For example, in some embodiments, R^{1b} is C₅ alkyl.

In some embodiments, R¹ is different from —(CHR⁵R⁶)ₘM-CR²R³R⁷.

In some embodiments, —CHR^{1a}R^{1b}— is different from —(CHR⁵R⁶)ₘM-CR²R³R⁷.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, Cis alkyl, and Cis alkenyl.

In some embodiments, R^{b′} is C₁₋₁₄ alkyl. In some embodiments, R^{b′} is C₂₋₁₄ alkyl. In some embodiments, R^{b′} is C₃₋₁₄ alkyl. In some embodiments, R^{b′} is C₁₋₈ alkyl. In some embodiments, R^{b′} is C₁₋₅ alkyl. In some embodiments, R^{b′} is C₁₋₃ alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

In one embodiment, the compounds of Formula (I) are of Formula (IIa), also referred to as Formula (I IIa):

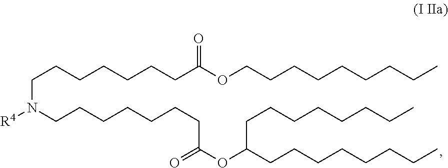

or their N-oxides, or salts or isomers thereof, wherein R⁴ is as described herein.

In another embodiment, the compounds of Formula (I) are of Formula (IIb), also referred to as Formula (I IIb):

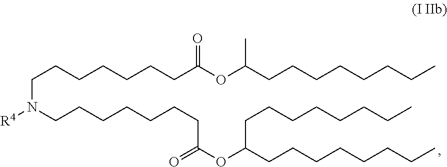

or their N-oxides, or salts or isomers thereof, wherein R⁴ is as described herein.

In another embodiment, the compounds of Formula (I) are of Formula (IIc) or (IIe), also referred to as Formula (I IIc) or (I IIe):

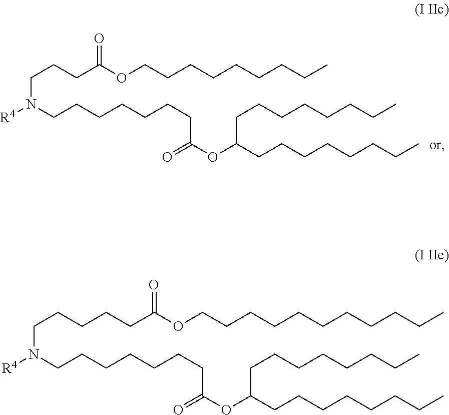

or their N-oxides, or salts or isomers thereof, wherein R⁴ is as described herein.

In another embodiment, the compounds of Formula (I I) are of Formula (I IIf):

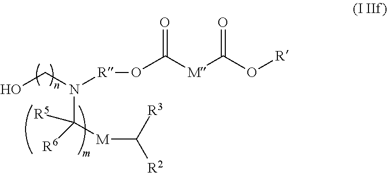

or their N-oxides, or salts or isomers thereof,

wherein M is —C(O)O— or —OC(O)—, M″ is C₁₋₆ alkyl or C₂₋₆ alkenyl, R² and R³ are independently selected from the group consisting of C₅₋₁₄ alkyl and C₅₋₁₄ alkenyl, and n is selected from 2, 3, and 4.

In a further embodiment, the compounds of Formula (I I) are of Formula (IId):

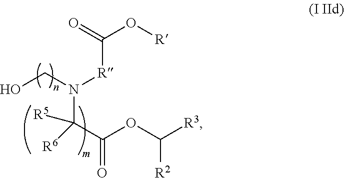

or their N-oxides, or salts or isomers thereof, wherein n is 2, 3, or 4; and m, R′, R″, and R² through R₆ are as described herein. For example, each of R² and R³ may be independently selected from the group consisting of C₅₋₁₄ alkyl and C₅₋₁₄ alkenyl.

In a further embodiment, the compounds of Formula (I) are of Formula (IIg), also referred to as Formula (I IIg):

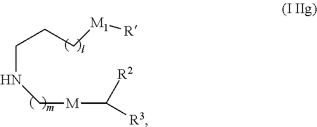

or their N-oxides, or salts or isomers thereof, wherein 1 is selected from 1, 2, 3, 4, and 5; m is selected from 5, 6, 7, 8, and 9; M₁ is a bond or M′; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R² and R³ are independently selected from the group consisting of C₅₋₁₄ alkyl and C₅₋₁₄ alkenyl.

In another embodiment, a subset of compounds of Formula (I VI) includes those of Formula (I VIIa):

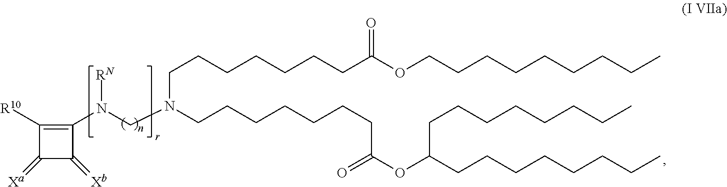

### or its N-oxide, or

a salt or isomer thereof.

In another embodiment, a subset of compounds of Formula (I VI) includes those of Formula (I VIIIa):

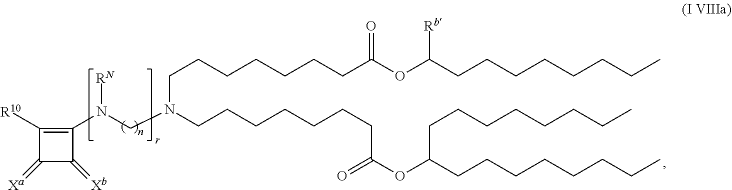

### or its N-oxide, or

a salt or isomer thereof.

In another embodiment, a subset of compounds of Formula (I VI) includes those of Formula (I VIIIb):

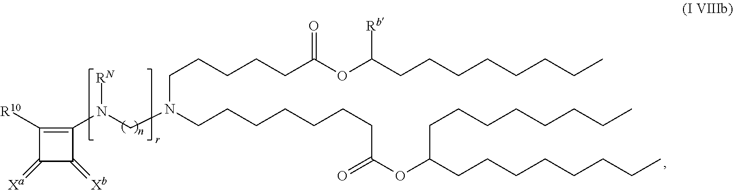

### or its N-oxide, or

a salt or isomer thereof.

In another embodiment, a subset of compounds of Formula (I VI) includes those of Formula (I VIIb-1):

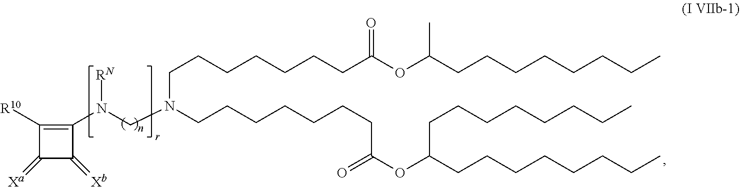

### or its N-oxide,

or a salt or isomer thereof.

In another embodiment, a subset of compounds of Formula (I VI) includes those of Formula (I VIIb-2):

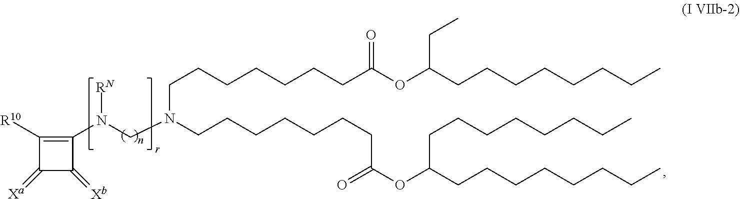

or its N-oxide, or a salt or isomer thereof.

In another embodiment, a subset of compounds of Formula (I VI) includes those of Formula (I VIIb-3):

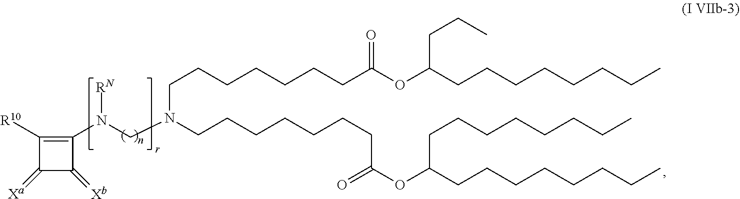

or its N-oxide, or a salt or isomer thereof. In another embodiment, a subset of compounds of Formula (VI) includes those of Formula (VIIc):

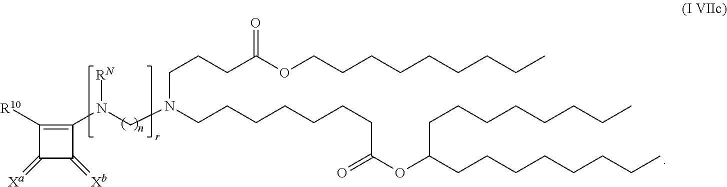

In another embodiment, a subset of compounds of Formula (I VI) includes those of Formula (VIId):

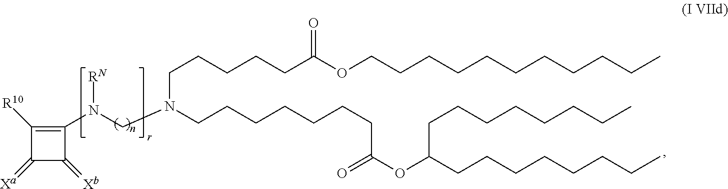

### or its N-oxide, or

a salt or isomer thereof.

In another embodiment, a subset of compounds of Formula (I VI) includes those of Formula (I VIIIc):

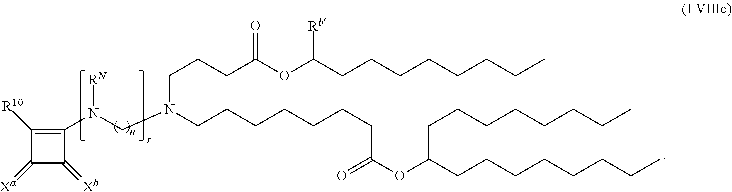

In another embodiment, a subset of compounds of Formula I VI) includes those of Formula (I VIIId):

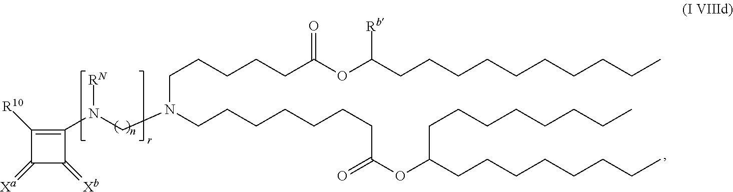

### or its N-oxide,

or a salt or isomer thereof.

The compounds of any one of formulae (I I), (I IA), (I IB), (I II), (I IIa), (I IIb), (I IIc), (I IId), (I IIe), (I IIf), (I IIg), I (I II), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), or (I VIIId) include one or more of the following features when applicable.

In some embodiments, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, 5- to 14-membered aromatic or non-aromatic heterocycle having one or more heteroatoms selected from N, O, S, and P, —OR, —O(CH₂)ₙN(R)₂, —C(O)OR, —OC(O)R, —CX₃, —CX₂H, —CXH₂, —CN, —N(R)₂, —N(R)S(O)₂R⁸, —C(O)N(R)₂, —N(R)C(O)R, —N(R)S(O)₂R, —N(R)C(O)N(R)₂, —N(R)C(S)N(R)₂, and —C(R)N(R)₂C(O)OR, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, each o is independently selected from 1, 2, 3, and 4, and each n is independently selected from 1, 2, 3, 4, and 5.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5; and when Q is a 5- to 14-membered heterocycle and (i) R⁴ is —(CH₂)ₙQ in which n is 1 or 2, or (ii) R⁴ is —(CH₂)ₙCHQR in which n is 1, or (iii) R⁴ is —CHQR, and —CQ(R)₂, then Q is either a 5- to 14-membered heteroaryl or 8- to 14-membered heterocycloalkyl.

In another embodiment, R⁴ is selected from the group consisting of a C₃₋₆ carbocycle, —(CH₂)ₙQ, —(CH₂)ₙCHQR, —(CH₂)ₒC(R¹⁰)₂(CH₂)ₙ₋ₒQ, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)S(O)₂R⁸, —N(R)C(O)R, —N(R)S(O)₂R, —N(R)C(O)N(R)₂, —N(R)C(S)N(R)₂, —C(R)N(R)₂C(O)OR, each o is independently selected from 1, 2, 3, and 4, and each n is independently selected from 1, 2, 3, 4, and 5.

In another embodiment, R⁴ is —(CH₂)ₙQ, where Q is —N(R)S(O)₂R⁸ and n is selected from 1, 2, 3, 4, and 5. In a further embodiment, R⁴ is —(CH₂)ₙQ, where Q is —N(R)S(O)₂R⁸, in which R⁸ is a C₃₋₆ carbocycle such as C₃₋₆ cycloalkyl, and n is selected from 1, 2, 3, 4, and 5. For example, R⁴ is —(CH₂)₃NHS(O)₂R⁸ and R⁸ is cyclopropyl.

In another embodiment, R⁴ is —(CH₂)ₒC(R¹⁰)₂(CH₂)ₙ₋ₒQ, where Q is —N(R)C(O)R, n is selected from 1, 2, 3, 4, and 5, and o is selected from 1, 2, 3, and 4. In a further embodiment, R⁴ is —(CH₂)ₒC(R¹⁰)₂(CH₂)ₙ₋ₒQ, where Q is —N(R)C(O)R, wherein R is C₁-C₃ alkyl and n is selected from 1, 2, 3, 4, and 5, and o is selected from 1, 2, 3, and 4. In a another embodiment, R⁴ is is —(CH₂)ₒC(R¹⁰)₂(CH₂)ₙ₋ₒQ, where Q is —N(R)C(O)R, wherein R is C₁-C₃ alkyl, n is 3, and o is 1. In some embodiments, R¹⁰ is H, OH, C₁₋₃ alkyl, or C₂₋₃ alkenyl. For example, R⁴ is 3-acetamido-2,2-dimethylpropyl.

In some embodiments, one R¹⁰ is H and one R¹⁰ is C₁₋₃ alkyl or C₂₋₃ alkenyl. In another embodiment, each R¹⁰ is is C₁₋₃ alkyl or C₂₋₃ alkenyl. In another embodiment, each R¹⁰ is is C₁₋₃ alkyl (e.g. methyl, ethyl or propyl). For example, one R¹⁰ is methyl and one R¹⁰ is ethyl or propyl. For example, one R¹⁰ is ethyl and one R¹⁰ is methyl or propyl. For example, one R¹⁰ is propyl and one R¹⁰ is methyl or ethyl. For example, each R¹⁰ is methyl. For example, each R¹⁰ is ethyl. For example, each R¹⁰ is propyl.

In some embodiments, one R¹⁰ is H and one R¹⁰ is OH. In another embodiment, each R¹⁰ is is OH.

In another embodiment, R⁴ is unsubstituted C₁₋₄ alkyl, e.g., unsubstituted methyl.

In another embodiment, R⁴ is hydrogen.

In certain embodiments, the disclosure provides a compound having the Formula (I), wherein R⁴ is —(CH₂)ₙQ or —(CH₂)ₙCHQR, where Q is —N(R)₂, and n is selected from 3, 4, and 5.

In certain embodiments, the disclosure provides a compound having the Formula (I), wherein R⁴ is selected from the group consisting of —(CH₂)ₙQ, —(CH₂)ₙCHQR, —CHQR, and —CQ(R)₂, where Q is —N(R)₂, and n is selected from 1, 2, 3, 4, and 5.

In certain embodiments, the disclosure provides a compound having the Formula (I), wherein R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle, and R⁴ is —(CH₂)ₙQ or —(CH₂)ₙCHQR, where Q is —N(R)₂, and n is selected from 3, 4, and 5.

In certain embodiments, R² and R³ are independently selected from the group consisting of C₂₋₁₄ alkyl, C₂₋₁₄ alkenyl, —R*YR″, —YR″, and —R*OR″, or R² and R³, together with the atom to which they are attached, form a heterocycle or carbocycle. In some embodiments, R² and R³ are independently selected from the group consisting of C₂₋₁₄ alkyl, and C₂₋₁₄ alkenyl. In some embodiments, R² and R³ are independently selected from the group consisting of —R*YR″, —YR″, and —R*OR″. In some embodiments, R² and R³ together with the atom to which they are attached, form a heterocycle or carbocycle.

In some embodiments, R¹ is selected from the group consisting of C₅₋₂₀ alkyl and C₅₋₂₀ alkenyl. In some embodiments, R¹ is C₅₋₂₀ alkyl substituted with hydroxyl.

In other embodiments, R¹ is selected from the group consisting of —R*YR″, —YR″, and —R″M′R′.

In certain embodiments, R¹ is selected from —R*YR″ and —YR″. In some embodiments, Y is a cyclopropyl group. In some embodiments, R* is C₈ alkyl or C₈ alkenyl. In certain embodiments, R″ is C₃₋₁₂ alkyl. For example, R″ may be C₃ alkyl. For example, R″ may be C₄₋₈ alkyl (e.g., C₄, C₅, C₆, C₇, or C₈ alkyl).

In some embodiments, R is (CH₂)_{q}OR*, q is selected from 1, 2, and 3, and R* is C₁₋₁₂ alkyl substituted with one or more substituents selected from the group consisting of amino, C₁-C₆alkylamino, and C₁-C₆ dialkylamino. For example, R is (CH₂)_{q}OR*, q is selected from 1, 2, and 3 and R* is C₁₋₁₂ alkyl substituted with C₁-C₆ dialkylamino. For example, R is (CH₂)_{q}OR*, q is selected from 1, 2, and 3 and R* is C₁₋₃ alkyl substituted with C₁-C₆ dialkylamino. For example, R is (CH₂)_{q}OR*, q is selected from 1, 2, and 3 and R* is C₁₋₃ alkyl substituted with dimethylamino (e.g., dimethylaminoethanyl).

In some embodiments, R¹ is C₅₋₂₀ alkyl. In some embodiments, R¹ is C₆ alkyl. In some embodiments, R¹ is C₈ alkyl. In other embodiments, R¹ is C₉ alkyl. In certain embodiments, R¹ is C₁₄ alkyl. In other embodiments, R¹ is C₁₈ alkyl.

In some embodiments, R¹ is C₂₁₋₃₀ alkyl. In some embodiments, R¹ is C₂₆ alkyl. In some embodiments, R¹ is C₂₈ alkyl. In certain embodiments, R¹ is

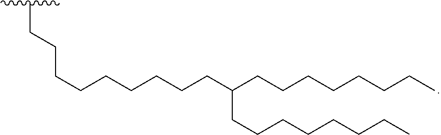 [SMILES: *CCCCCCCCCC(CCCCCCCC)CCCCCCCC]

In some embodiments, R¹ is C₅₋₂₀ alkenyl. In certain embodiments, R¹ is C₁₈ alkenyl. In some embodiments, R¹ is linoleyl.

In certain embodiments, R¹ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl, or heptadeca-9-yl). In certain embodiments, R¹ is

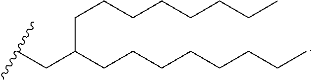 [SMILES: *CC(CCCCCCCC)CCCCCCCC]

In certain embodiments, R¹ is unsubstituted C₅₋₂₀ alkyl or C₅₋₂₀ alkenyl. In certain embodiments, R′ is substituted C₅₋₂₀ alkyl or C₅₋₂₀ alkenyl (e.g., substituted with a C₃₋₆ carbocycle such as 1-cyclopropylnonyl or substituted with OH or alkoxy). For example, R¹ is

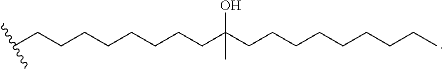 [SMILES: *CCCCCCCCC(C)(O)CCCCCCCCC]

In other embodiments, R¹ is —R″M′R′. In certain embodiments, M′ is —OC(O)-M″-C(O)O—. For example, R¹ is

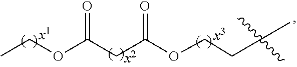

wherein x¹ is an integer between 1 and 13 (e.g., selected from 3, 4, 5, and 6), x² is an integer between 1 and 13 (e.g., selected from 1, 2, and 3), and x³ is an integer between 2 and 14 (e.g., selected from 4, 5, and 6). For example, x¹ is selected from 3, 4, 5, and 6, x² is selected from 1, 2, and 3, and x³ is selected from 4, 5, and 6.

In other embodiments, R¹ is different from —(CHR⁵R⁶)ₘM-CR²R³R⁷.

In some embodiments, R′ is selected from —R*YR″ and —YR″. In some embodiments, Y is C₃₋₈ cycloalkyl. In some embodiments, Y is C₆₋₁₀ aryl. In some embodiments, Y is a cyclopropyl group. In some embodiments, Y is a cyclohexyl group. In certain embodiments, R* is C₁ alkyl.

In some embodiments, R″ is selected from the group consisting of C₃₋₁₂ alkyl and C₃₋₁₂ alkenyl. In some embodiments, R″ is C₈ alkyl. In some embodiments, R″ adjacent to Y is C₁ alkyl. In some embodiments, R″ adjacent to Y is C₄₋₉ alkyl (e.g., C₄, C₅, C₆, C₇ or C₈ or C₉ alkyl).

In some embodiments, R″ is substituted C₃₋₁₂ (e.g., C₃₋₁₂ alkyl substituted with, e.g., an hydroxyl). For example, R″ is OH

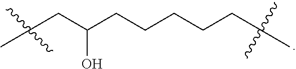 [SMILES: *CCCCCC(O)C*]

In some embodiments, R′ is selected from C₄ alkyl and C₄ alkenyl. In certain embodiments, R′ is selected from C₅ alkyl and C₅ alkenyl. In some embodiments, R′ is selected from C₆ alkyl and C₆ alkenyl. In some embodiments, R′ is selected from C₇ alkyl and C₇ alkenyl. In some embodiments, R′ is selected from C₉ alkyl and C₉ alkenyl.

In some embodiments, R′ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, Cis alkyl, and Cis alkenyl, each of which is either linear or branched.

In some embodiments, R′ is linear. In some embodiments, R′ is branched.

In some embodiments, R′ is

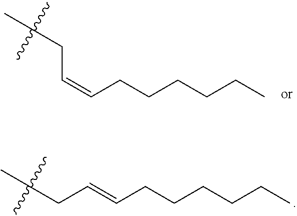

In some embodiments, R′ is

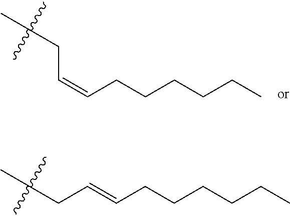

and M′ is —OC(O)—. In other embodiments, R′ is

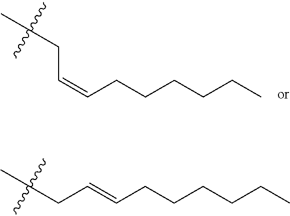

### and M′ is —C(O)O—.

In other embodiments, R′ is selected from C₁₁ alkyl and C₁₁ alkenyl. In other embodiments, R′ is selected from C₁₂ alkyl, C₁₂ alkenyl, C₁₃ alkyl, C₁₃ alkenyl, C₁₄ alkyl, C₁₄ alkenyl, C₁₅ alkyl, C₁₅ alkenyl, C₁₆ alkyl, C₁₆ alkenyl, C₁₇ alkyl, C₁₇ alkenyl, Cis alkyl, and Cis alkenyl. In certain embodiments, R′ is linear C₄₋₁₈ alkyl or C₄₋₁₈ alkenyl. In certain embodiments, R′ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl or heptadeca-9-yl). In certain embodiments, R′ is

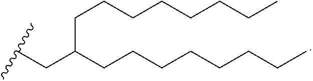 [SMILES: *CC(CCCCCCCC)CCCCCCCC]

In certain embodiments, R′ is unsubstituted C₁₋₁₈ alkyl. In certain embodiments, R′ is substituted C₁₋₁₈ alkyl (e.g., C₁₋₁₅ alkyl substituted with, e.g., an alkoxy such as methoxy, or a C₃₋₆ carbocycle such as 1-cyclopropylnonyl, or C(O)O-alkyl or OC(O)-alkyl such as C(O)OCH₃ or OC(O)CH₃). For example, R′ is

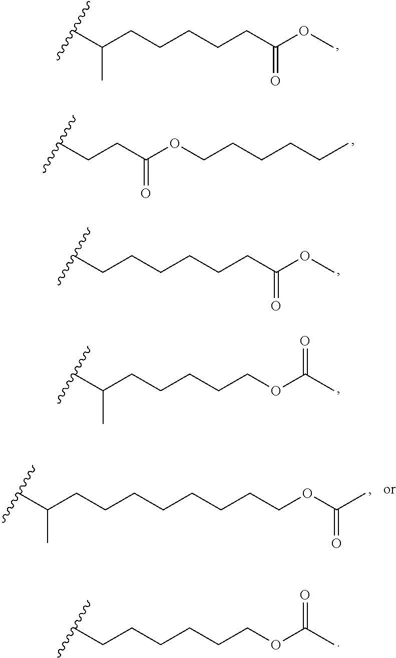

In certain embodiments, R′ is branched C₁₋₁₈ alkyl. For example, R′ is

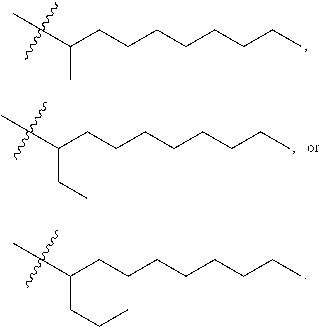 [SMILES: *C(C)CCCCCCCC ; *C(CC)CCCCCCCC ; *C(CCC)CCCCCCCC]

In some embodiments, R″ is selected from the group consisting of C₃₋₁₅ alkyl and C₃₋₁₅ alkenyl. In some embodiments, R″ is C₃ alkyl, C₄ alkyl, C₅ alkyl, C₆ alkyl, C₇ alkyl, or C₈ alkyl. In some embodiments, R″ is C₉ alkyl, C₁₀ alkyl, C₁₁ alkyl, C₁₂ alkyl, C₁₃ alkyl, C₁₄ alkyl, or C₁₅ alkyl.

In some embodiments, M′ is —C(O)O—. In some embodiments, M′ is —OC(O)—. In some embodiments, M′ is —OC(O)-M″-C(O)O—.

In some embodiments, M′ is —C(O)O—, —OC(O)—, or —OC(O)-M″-C(O)O—. In some embodiments wherein M′ is —OC(O)-M″-C(O)O—, M″ is C₁₋₄ alkyl or C₂₋₄ alkenyl.

In other embodiments, M′ is an aryl group or heteroaryl group. For example, M′ may be selected from the group consisting of phenyl, oxazole, and thiazole.

In some embodiments, M is —C(O)O—. In some embodiments, M is —OC(O)—. In some embodiments, M is —C(O)N(R′)—. In some embodiments, M is —P(O)(OR′)O—. In some embodiments, M is —OC(O)-M″-C(O)O—.

In some embodiments, M is —C(O). In some embodiments, M is —OC(O)— and M′ is —C(O)O—. In some embodiments, M is —C(O)O— and M′ is —OC(O)—. In some embodiments, M and M′ are each —OC(O)—. In some embodiments, M and M′ are each —C(O)O—.

In other embodiments, M is an aryl group or heteroaryl group. For example, M may be selected from the group consisting of phenyl, oxazole, and thiazole.

In some embodiments, M is the same as M′. In other embodiments, M is different from M′.

In some embodiments, M″ is a bond. In some embodiments, M″ is C₁₋₁₃ alkyl or C₂₋₁₃ alkenyl. In some embodiments, M″ is C₁₋₆ alkyl or C₂₋₆ alkenyl. In certain embodiments, M″ is linear alkyl or alkenyl. In certain embodiments, M″ is branched, e.g., —CH(CH₃)CH₂—.

In some embodiments, each R⁵ is H. In some embodiments, each R⁶ is H. In certain such embodiments, each R⁵ and each R⁶ is H.

In some embodiments, R⁷ is H. In other embodiments, R⁷ is C₁₋₃ alkyl (e.g., methyl, ethyl, propyl, or i-propyl).

In some embodiments, R² and R³ are independently C₅₋₁₄ alkyl or C₅₋₁₄ alkenyl.

In some embodiments, R² and R³ are the same. In some embodiments, R² and R³ are C₈ alkyl. In certain embodiments, R² and R³ are C₂ alkyl. In other embodiments, R² and R³ are C₃ alkyl. In some embodiments, R² and R³ are C₄ alkyl. In certain embodiments, R² and R³ are C₅ alkyl. In other embodiments, R² and R³ are C₆ alkyl. In some embodiments, R² and R³ are C₇ alkyl.

In other embodiments, R² and R³ are different. In certain embodiments, R² is C₈ alkyl. In some embodiments, R³ is C₁₋₇ (e.g., C₁, C₂, C₃, C₄, C₅, C₆, or C₇ alkyl) or C₉ alkyl.

In some embodiments, R³ is C₁ alkyl. In some embodiments, R³ is C₂ alkyl. In some embodiments, R³ is C₃ alkyl. In some embodiments, R³ is C₄ alkyl. In some embodiments, R³ is C₅ alkyl. In some embodiments, R³ is C₆ alkyl. In some embodiments, R³ is C₇ alkyl. In some embodiments, R³ is C₉ alkyl.

In some embodiments, R⁷ and R³ are H.

In certain embodiments, R² is H.

In some embodiments, m is 5, 6, 7, 8, or 9. In some embodiments, m is 5, 7, or 9. For example, in some embodiments, m is 5. For example, in some embodiments, m is 7. For example, in some embodiments, m is 9.

In some embodiments, R⁴ is selected from —(CH₂)ₙQ and —(CH₂)ₙCHQR.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), —C(R)N(R)₂C(O)OR, —N(R)S(O)₂R⁸, a carbocycle, and a heterocycle.

In certain embodiments, Q is —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, or —N(R)C(O)OR.

In certain embodiments, Q is —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, or —N(OR)C(═CHR⁹)N(R)₂.

In certain embodiments, Q is thiourea or an isostere thereof, e.g.,

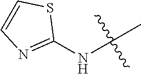 [SMILES: *Nc1nccs1]

or —NHC(═NR⁹)N(R)₂.

In certain embodiments, Q is —C(═NR⁹)N(R)₂. For example, when Q is —C(═NR⁹)N(R)₂,

n is 4 or 5. For example, R⁹ is —S(O)₂N(R)₂.

In certain embodiments, Q is —C(═NR⁹)R or —C(O)N(R)OR, e.g., —CH(═N—OCH₃), —C(O)NH—OH, —C(O)NH—OCH₃, —C(O)N(CH₃)—OH, or —C(O)N(CH₃)—OCH₃.

In certain embodiments, Q is —OH.

In certain embodiments, Q is a substituted or unsubstituted 5- to 10-membered heteroaryl, e.g., Q is a triazole, an imidazole, a pyrimidine, a purine, 2-amino-1,9-dihydro-6H-purin-6-one-9-yl (or guanin-9-yl), adenin-9-yl, cytosin-1-yl, or uracil-1-yl, each of which is optionally substituted with one or more substituents selected from alkyl, OH, alkoxy, -alkyl-OH, -alkyl-O-alkyl, and the substituent can be further substituted. In certain embodiments, Q is a substituted 5- to 14-membered heterocycloalkyl, e.g., substituted with one or more substituents selected from oxo (═O), OH, amino, mono- or di-alkylamino, and C₁₋₃ alkyl. For example, Q is 4-methylpiperazinyl, 4-(4-methoxybenzyl)piperazinyl, isoindolin-2-yl-1,3-dione, pyrrolidin-1-yl-2,5-dione, or imidazolidin-3-yl-2,4-dione.

In certain embodiments, Q is —NHR⁸, in which R⁸ is a C₃₋₆ cycloalkyl optionally substituted with one or more substituents selected from oxo (═O), amino (NH₂), mono- or di-alkylamino, C₁₋₃ alkyl and halo. For example, R⁸ is cyclobutenyl, e.g., 3-(dimethylamino)-cyclobut-3-ene-4-yl-1,2-dione. In further embodiments, R⁸ is a C₃₋₆ cycloalkyl optionally substituted with one or more substituents selected from oxo (═O), thio (═S), amino (NH₂), mono- or di-alkylamino, C₁₋₃ alkyl, heterocycloalkyl, and halo, wherein the mono- or di-alkylamino, C₁₋₃ alkyl, and heterocycloalkyl are further substituted. For example R⁸ is cyclobutenyl substituted with one or more of oxo, amino, and alkylamino, wherein the alkylamino is further substituted, e.g., with one or more of C₁₋₃ alkoxy, amino, mono- or di-alkylamino, and halo. For example, R⁸ is 3-(((dimethylamino)ethyl)amino)cyclobut-3-enyl-1,2-dione. For example R⁸ is cyclobutenyl substituted with one or more of oxo, and alkylamino. For example, R⁸ is 3-(ethylamino)cyclobut-3-ene-1,2-dione. For example R⁸ is cyclobutenyl substituted with one or more of oxo, thio, and alkylamino. For example R⁸ is 3-(ethylamino)-4-thioxocyclobut-2-en-1-one or 2-(ethylamino)-4-thioxocyclobut-2-en-1-one. For example R⁸ is cyclobutenyl substituted with one or more of thio, and alkylamino. For example R⁸ is 3-(ethylamino)cyclobut-3-ene-1,2-dithione. For example R⁸ is cyclobutenyl substituted with one or more of oxo and dialkylamino. For example R⁸ is 3-(diethylamino)cyclobut-3-ene-1,2-dione. For example, R⁸ is cyclobutenyl substituted with one or more of oxo, thio, and dialkylamino. For example, R⁸ is 2-(diethylamino)-4-thioxocyclobut-2-en-1-one or 3-(diethylamino)-4-thioxocyclobut-2-en-1-one. For example, R⁸ is cyclobutenyl substituted with one or more of thio, and dialkylamino. For example, R⁸ is 3-(diethylamino)cyclobut-3-ene-1,2-dithione. For example, R⁸ is cyclobutenyl substituted with one or more of oxo and alkylamino or dialkylamino, wherein alkylamino or dialkylamino is further substituted, e.g. with one or more alkoxy. For example, R⁸ is 3-(bis(2-methoxyethyl)amino)cyclobut-3-ene-1,2-dione. For example, R⁸ is cyclobutenyl substituted with one or more of oxo, and heterocycloalkyl. For example, R⁸ is cyclobutenyl substituted with one or more of oxo, and piperidinyl, piperazinyl, or morpholinyl. For example, R⁸ is cyclobutenyl substituted with one or more of oxo, and heterocycloalkyl, wherein heterocycloalkyl is further substituted, e.g., with one or more C₁₋₃ alkyl. For example, R⁸ is cyclobutenyl substituted with one or more of oxo, and heterocycloalkyl, wherein heterocycloalkyl (e.g., piperidinyl, piperazinyl, or morpholinyl) is further substituted with methyl.

In certain embodiments, Q is —NHR⁸, in which R⁸ is a heteroaryl optionally substituted with one or more substituents selected from amino (NH₂), mono- or di-alkylamino, C₁₋₃ alkyl and halo. For example, R⁸ is thiazole or imidazole.

In certain embodiments, Q is —NHC(═NR⁹)N(R)₂ in which R⁹ is CN, C₁₋₆ alkyl, NO₂, —S(O)₂N(R)₂, —OR, —S(O)₂R, or H. For example, Q is —NHC(═NR⁹)N(CH₃)₂, —NHC(═NR⁹)NHCH₃, —NHC(═NR⁹)NH₂. In some embodiments, Q is —NHC(═NR⁹)N(R)₂ in which R⁹ is CN and R is C₁₋₃ alkyl substituted with mono- or di-alkylamino, e.g., R is ((dimethylamino)ethyl)amino. In some embodiments, Q is —NHC(═NR⁹)N(R)₂ in which R⁹ is C₁₋₆ alkyl, NO₂, —S(O)₂N(R)₂, —OR, —S(O)₂R, or H and R is C₁₋₃ alkyl substituted with mono- or di-alkylamino, e.g., R is ((dimethylamino)ethyl)amino.

In certain embodiments, Q is —NHC(═CHR⁹)N(R)₂, in which R⁹ is NO₂, CN, C₁₋₆ alkyl, —S(O)₂N(R)₂, —OR, —S(O)₂R, or H. For example, Q is —NHC(═CHR⁹)N(CH₃)₂, —NHC(═CHR⁹)NHCH₃, or —NHC(═CHR⁹)NH₂.

In certain embodiments, Q is —OC(O)N(R)₂, —N(R)C(O)OR, —N(OR)C(O)OR, such as —OC(O)NHCH₃, —N(OH)C(O)OCH₃, —N(OH)C(O)CH₃, —N(OCH₃)C(O)OCH₃, —N(OCH₃)C(O)CH₃, —N(OH)S(O)₂CH₃, or —NHC(O)OCH₃.

In certain embodiments, Q is —N(R)C(O)R, in which R is alkyl optionally substituted with C₁₋₃ alkoxyl or S(O)_{z}C₁₋₃ alkyl, in which z is 0, 1, or 2.

In certain embodiments, Q is an unsubstituted or substituted C₆₋₁₀ aryl (such as phenyl) or C₃₋₆ cycloalkyl.

In some embodiments, n is 1. In other embodiments, n is 2. In further embodiments, n is 3. In certain other embodiments, n is 4. For example, R⁴ may be —(CH₂)₂OH. For example, R⁴ may be —(CH₂)₃OH. For example, R⁴ may be —(CH₂)₄OH. For example, R⁴ may be benzyl. For example, R⁴ may be 4-methoxybenzyl.

In some embodiments, R⁴ is a C₃₋₆ carbocycle. In some embodiments, R⁴ is a C₃₋₆ cycloalkyl. For example, R⁴ may be cyclohexyl optionally substituted with e.g., OH, halo, C₁₋₆ alkyl, etc. For example, R⁴ may be 2-hydroxycyclohexyl.

In some embodiments, R is H.

In some embodiments, R is C₁₋₃ alkyl substituted with mono- or di-alkylamino, e.g., R is ((dimethylamino)ethyl)amino.

In some embodiments, R is C₁₋₆ alkyl substituted with one or more substituents selected from the group consisting of C₁₋₃ alkoxyl, amino, and C₁-C₃ dialkylamino.

In some embodiments, R is unsubstituted C₁₋₃ alkyl or unsubstituted C₂₋₃ alkenyl. For example, R⁴ may be —CH₂CH(OH)CH₃, —CH(CH₃)CH₂OH, or —CH₂CH(OH)CH₂CH₃.

In some embodiments, R is substituted C₁₋₃ alkyl, e.g., CH₂OH. For example, R⁴ may be —CH₂CH(OH)CH₂OH, —(CH₂)₃NHC(O)CH₂OH, —(CH₂)₃NHC(O)CH₂OBn, —(CH₂)₂O(CH₂)₂OH, —(CH₂)₃NHCH₂OCH₃, —(CH₂)₃NHCH₂OCH₂CH₃, CH₂SCH₃, CH₂S(O)CH₃, CH₂S(O)₂CH₃, or —CH(CH₂OH)₂.

In some embodiments, R⁴ is selected from any of the following groups:

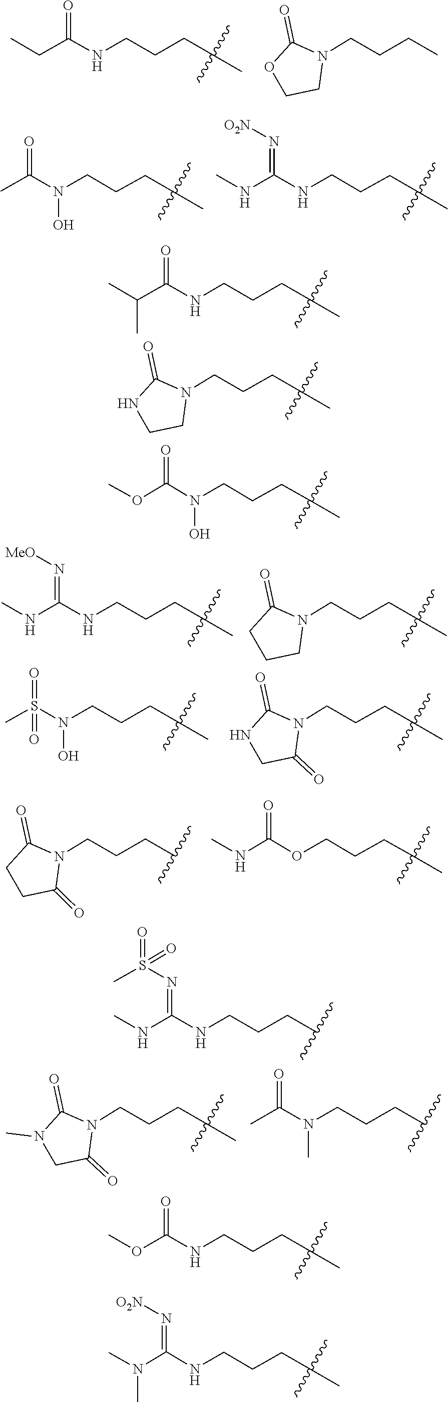

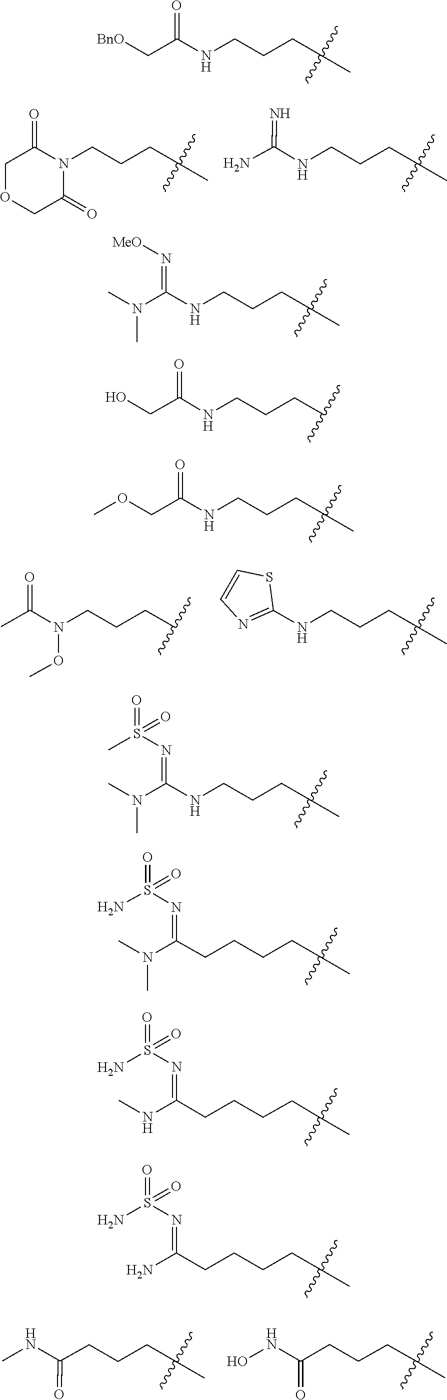

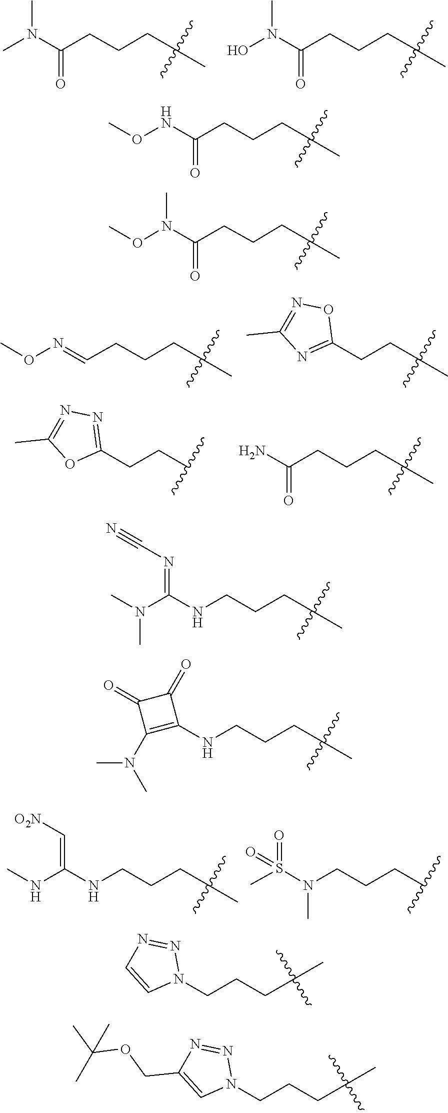

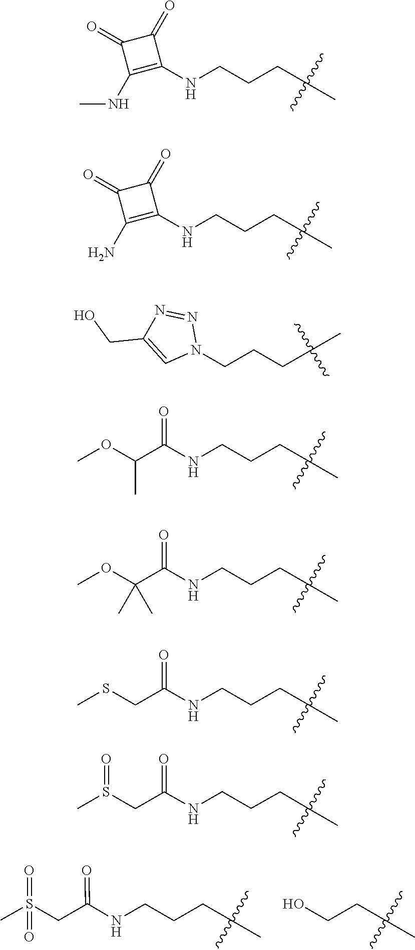

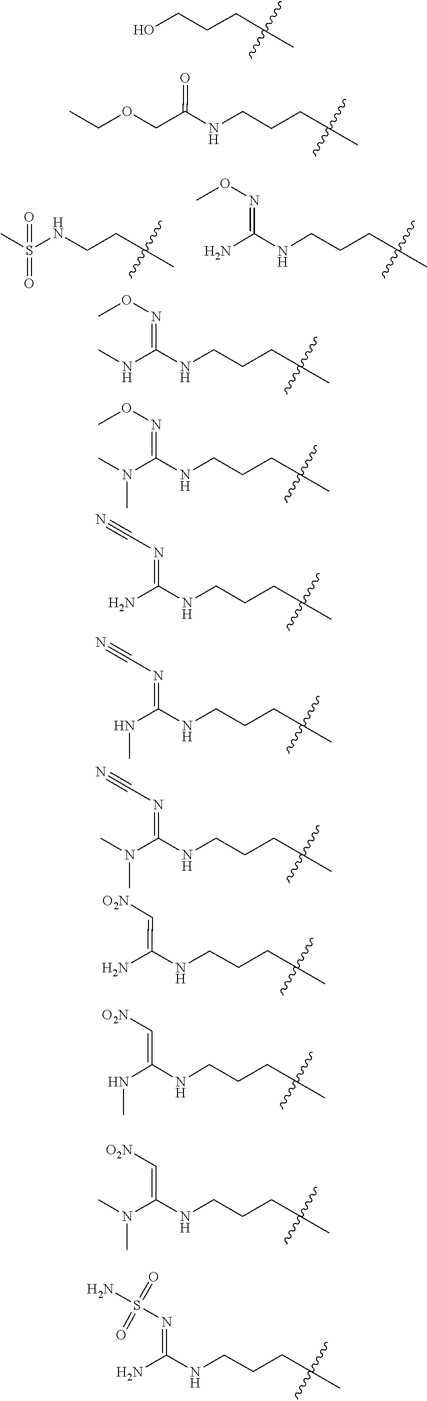

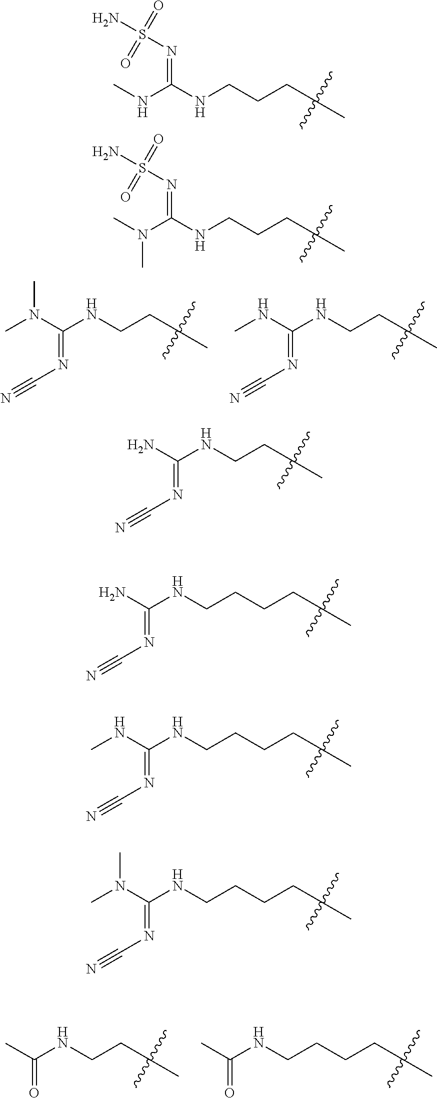

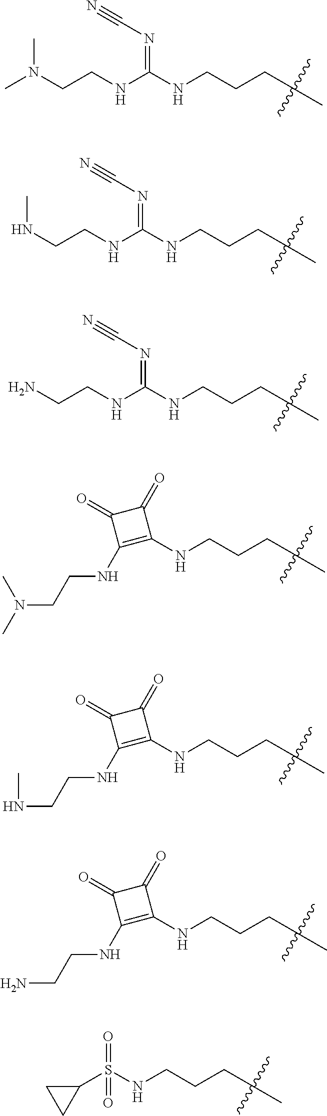

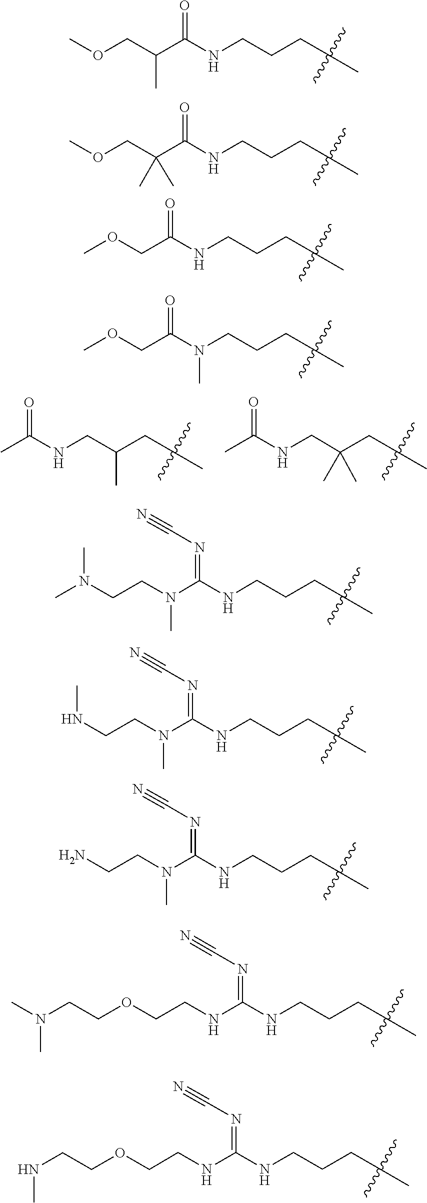

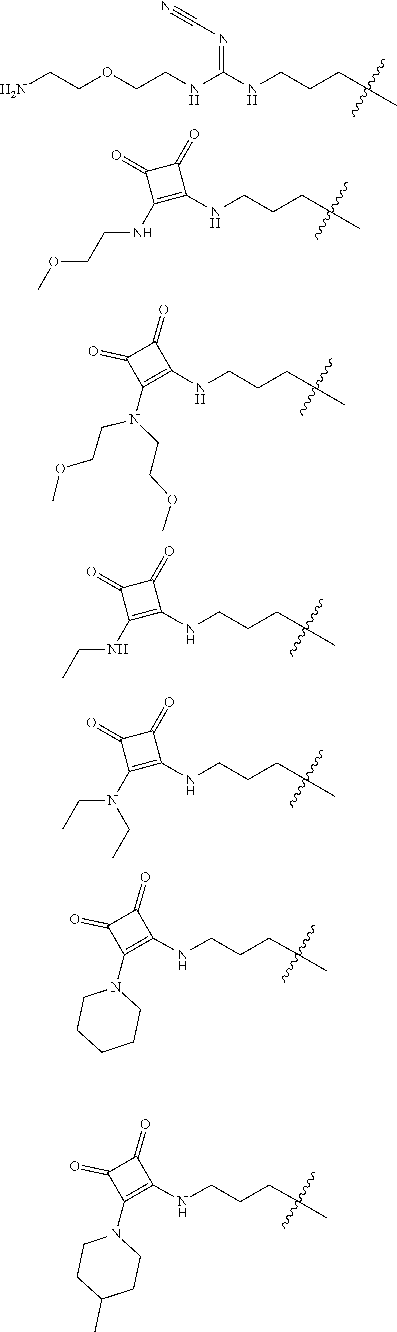

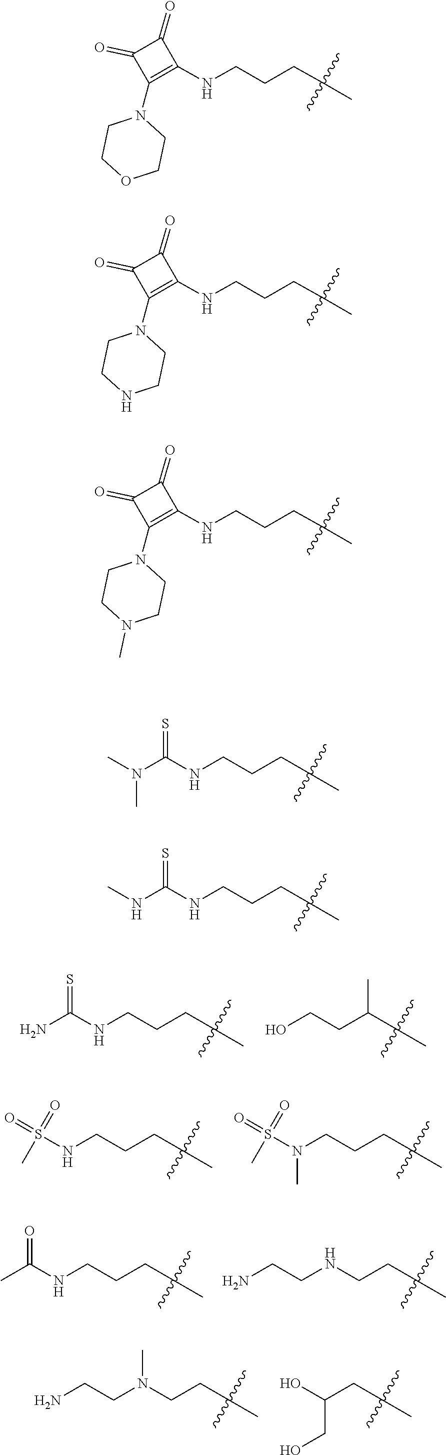

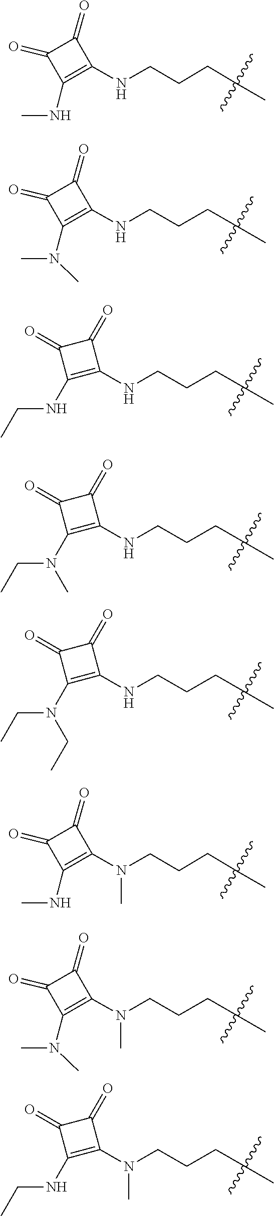

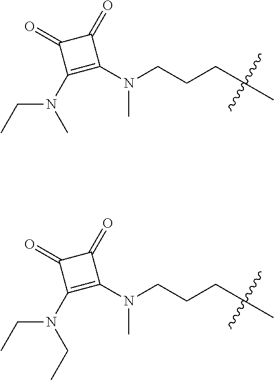 [SMILES: *CCCN(C)c1c(N(C)CC)c(=O)c1=O ; *CCCN(C)c1c(N(CC)CC)c(=O)c1=O]

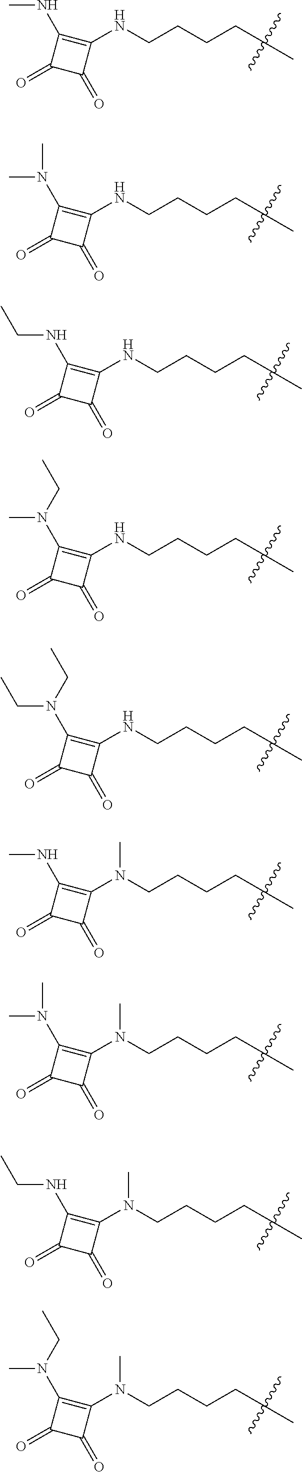

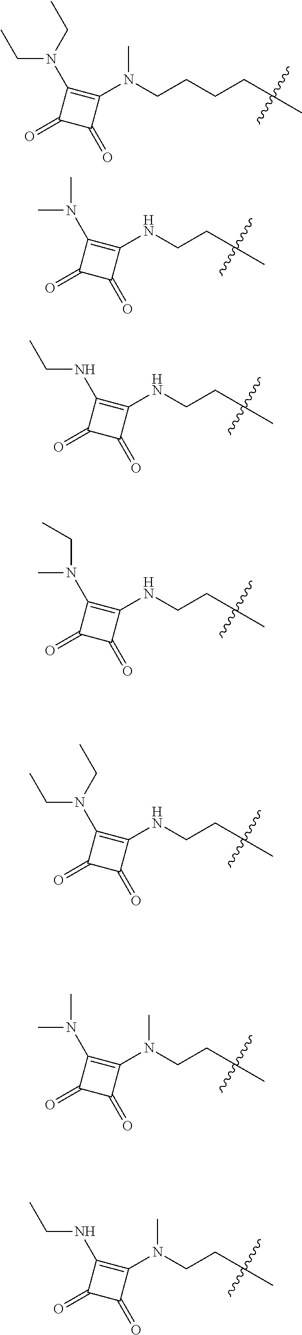

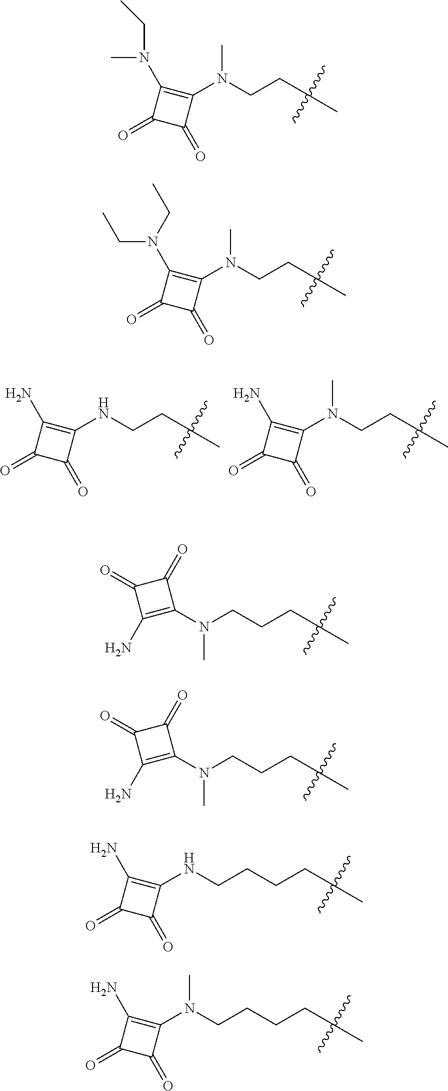

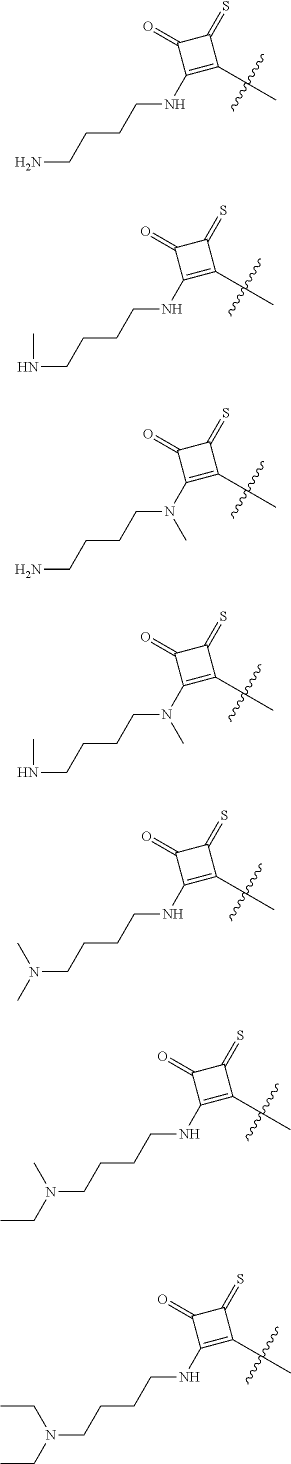

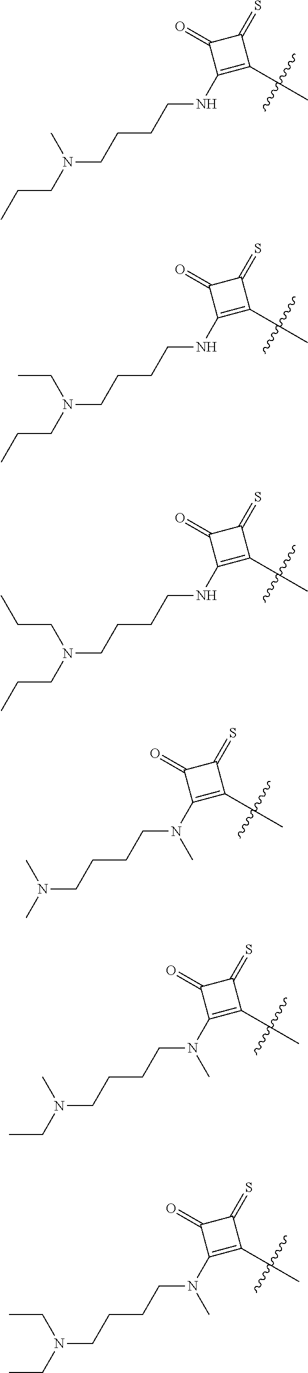 [SMILES: *c1c(NCCCCN(C)CCC)c(=O)c1=S ; *c1c(NCCCCN(CC)CCC)c(=O)c1=S ; *c1c(NCCCCN(CCC)CCC)c(=O)c1=S ; *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S]

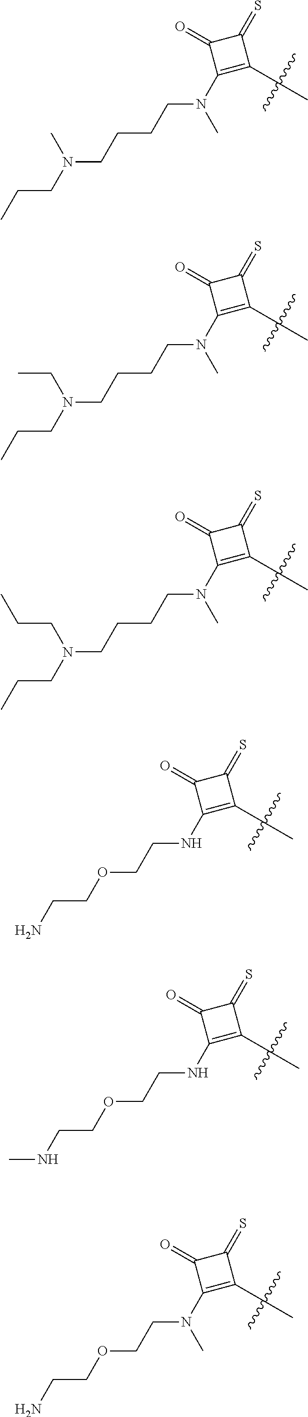 [SMILES: *c1c(N(C)CCCCN(C)CCC)c(=O)c1=S ; *c1c(N(C)CCCCN(CC)CCC)c(=O)c1=S ; *c1c(N(C)CCCCN(CCC)CCC)c(=O)c1=S ; *c1c(NCCOCCN)c(=O)c1=S ; *c1c(NCCOCCNC)c(=O)c1=S ; *c1c(N(C)CCOCCN)c(=O)c1=S]

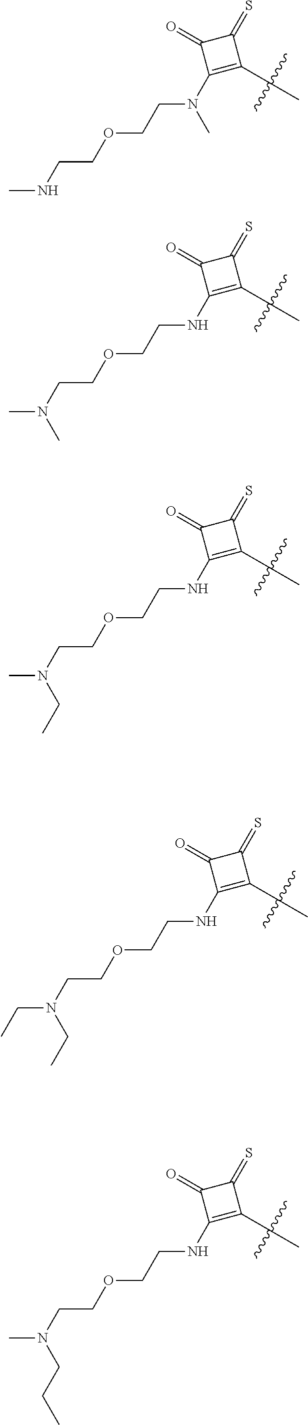 [SMILES: *c1c(N(C)CCOCCNC)c(=O)c1=S ; *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S]

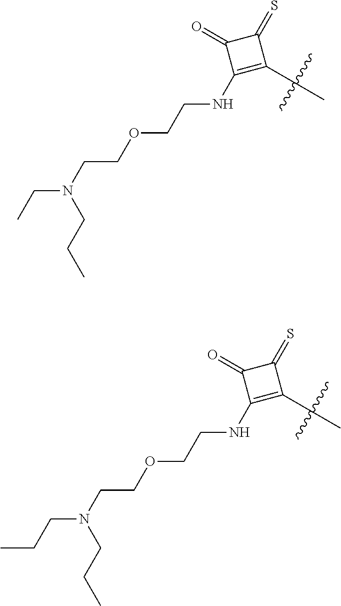 [SMILES: *c1c(NCCOCCN(CC)CCC)c(=O)c1=S ; *c1c(NCCOCCN(CCC)CCC)c(=O)c1=S]

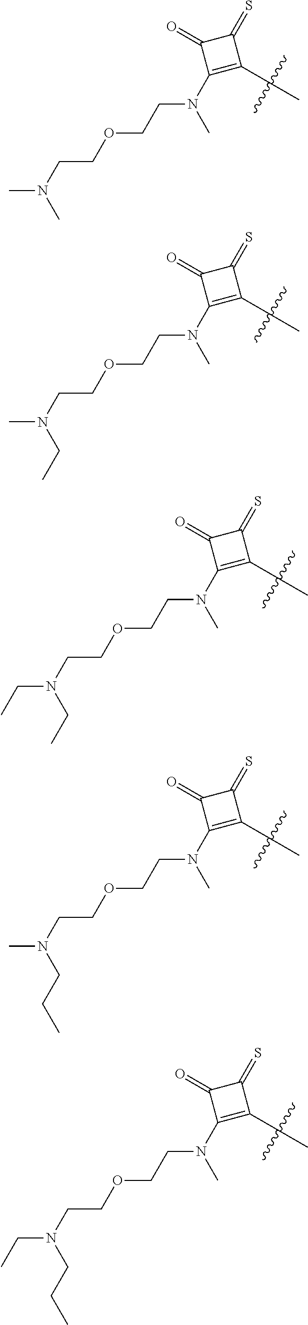 [SMILES: *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S ; *c1c(N(C)CCOCCN(CC)CCC)c(=O)c1=S]

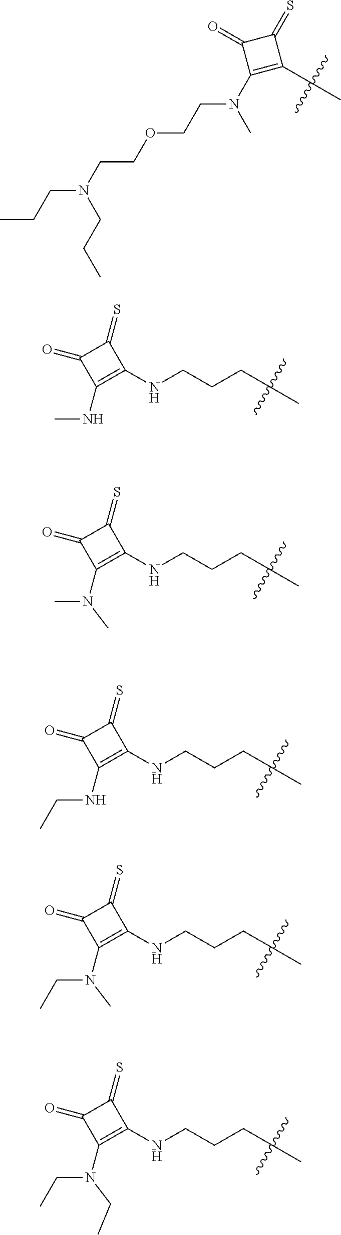

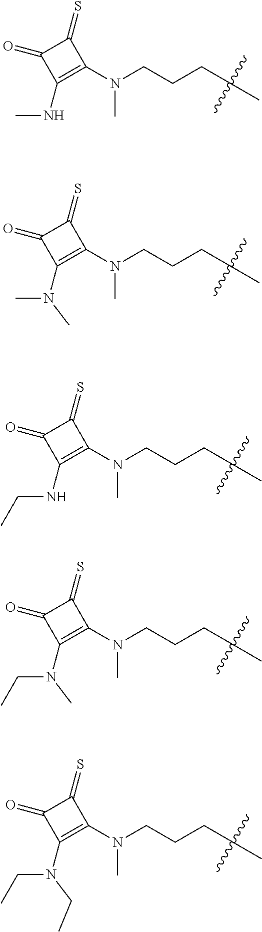 [SMILES: *CCCN(C)c1c(NC)c(=O)c1=S ; *CCCN(C)c1c(N(C)C)c(=O)c1=S ; *CCCN(C)c1c(NCC)c(=O)c1=S ; *CCCN(C)c1c(N(C)CC)c(=O)c1=S ; *CCCN(C)c1c(N(CC)CC)c(=O)c1=S]

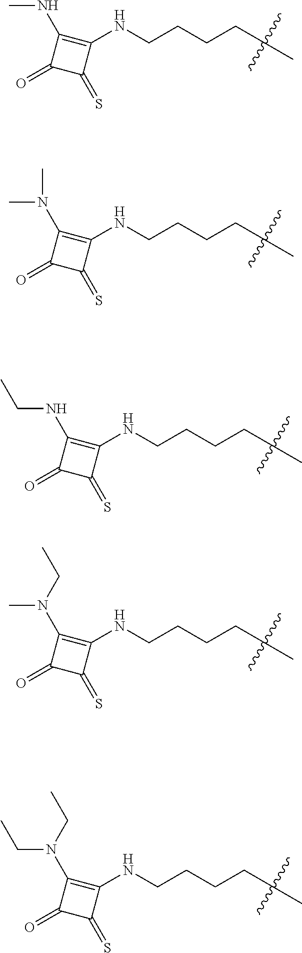 [SMILES: *CCCCNc1c(NC)c(=O)c1=S ; *CCCCNc1c(N(C)C)c(=O)c1=S ; *CCCCNc1c(NCC)c(=O)c1=S ; *CCCCNc1c(N(C)CC)c(=O)c1=S ; *CCCCNc1c(N(CC)CC)c(=O)c1=S]

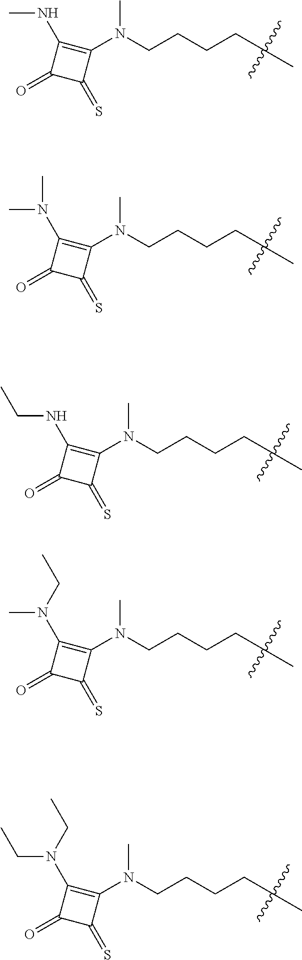 [SMILES: *CCCCN(C)c1c(NC)c(=O)c1=S ; *CCCCN(C)c1c(N(C)C)c(=O)c1=S ; *CCCCN(C)c1c(NCC)c(=O)c1=S ; *CCCCN(C)c1c(N(C)CC)c(=O)c1=S ; *CCCCN(C)c1c(N(CC)CC)c(=O)c1=S]

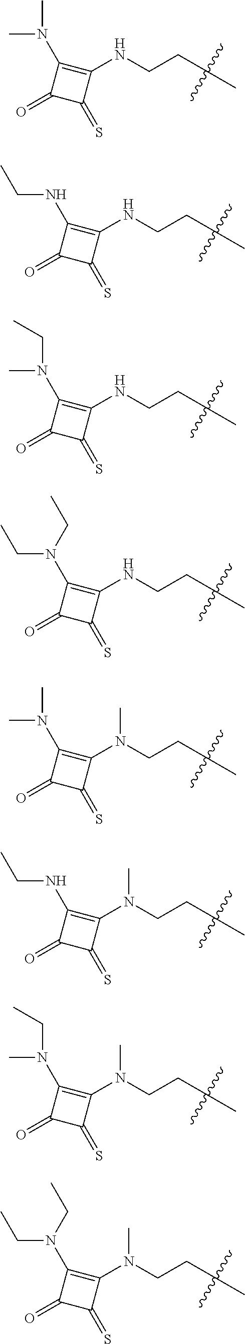

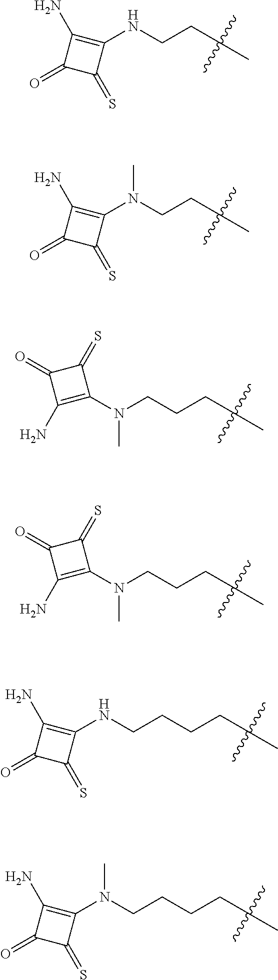 [SMILES: *CCNc1c(N)c(=O)c1=S ; *CCN(C)c1c(N)c(=O)c1=S ; *CCCN(C)c1c(N)c(=O)c1=S ; *CCCN(C)c1c(N)c(=O)c1=S ; *CCCCNc1c(N)c(=O)c1=S ; *CCCCN(C)c1c(N)c(=O)c1=S]

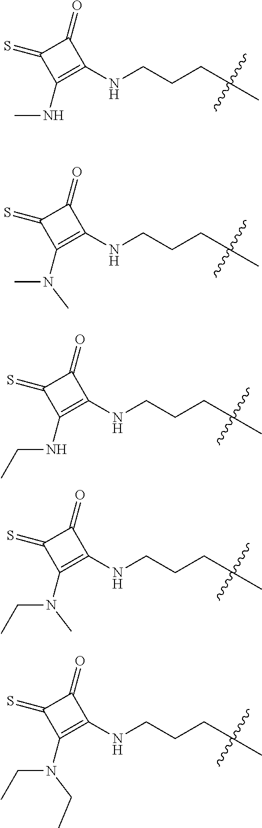

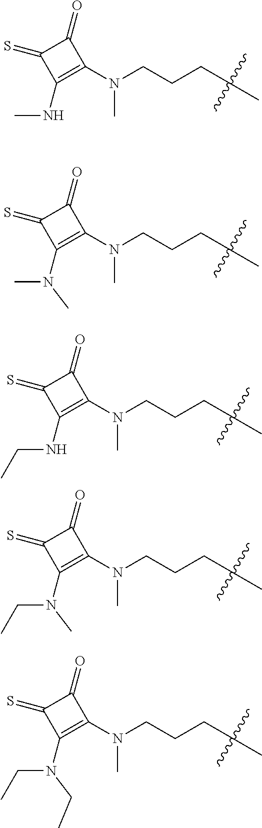

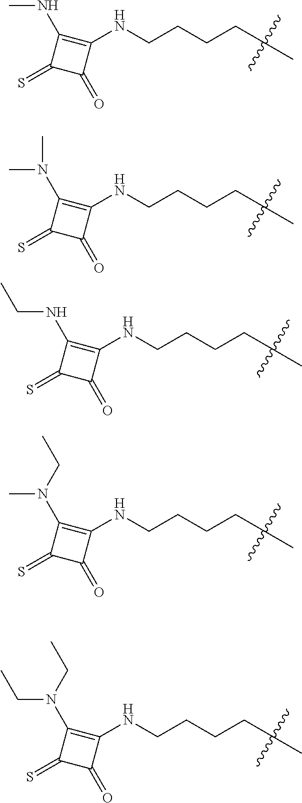

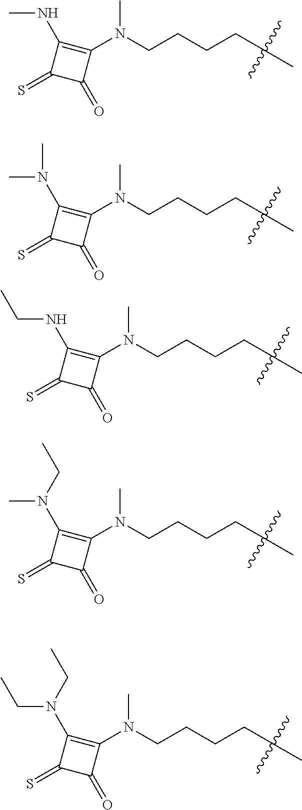

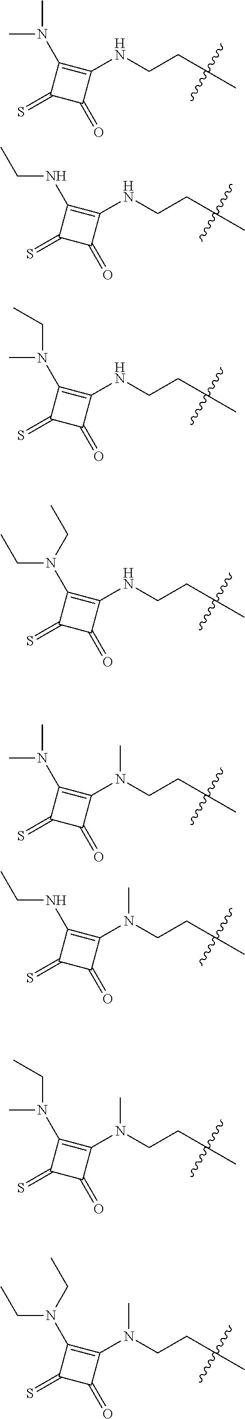

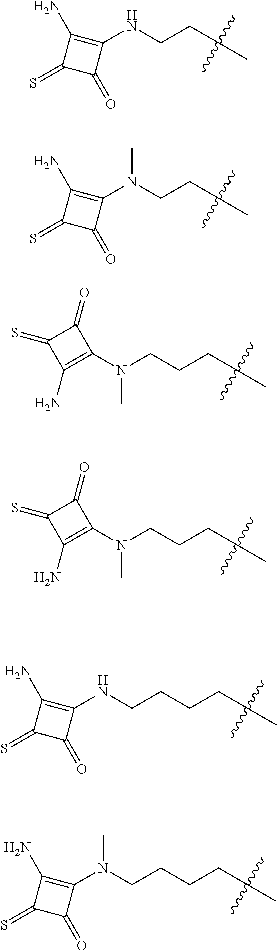

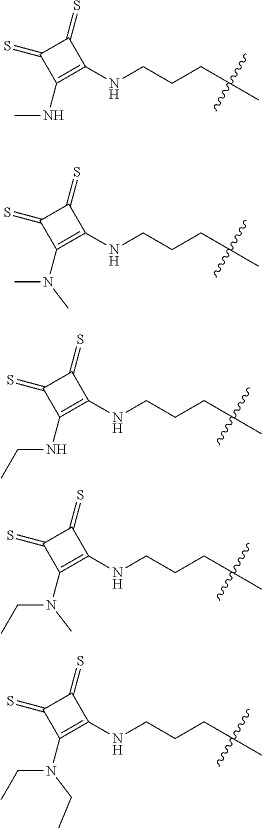

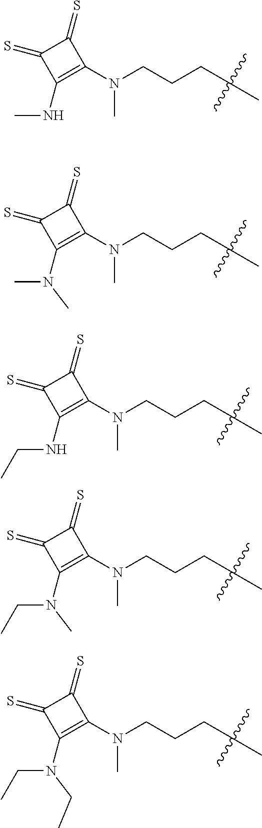

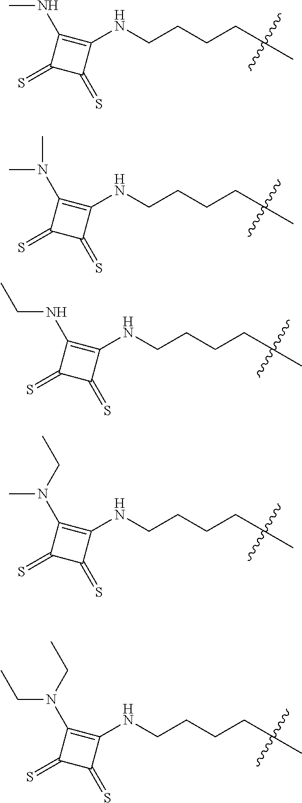

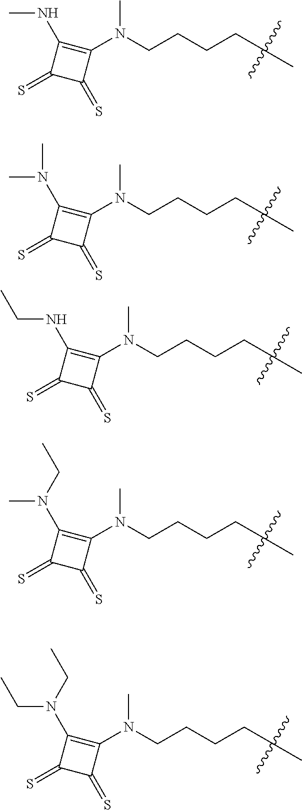

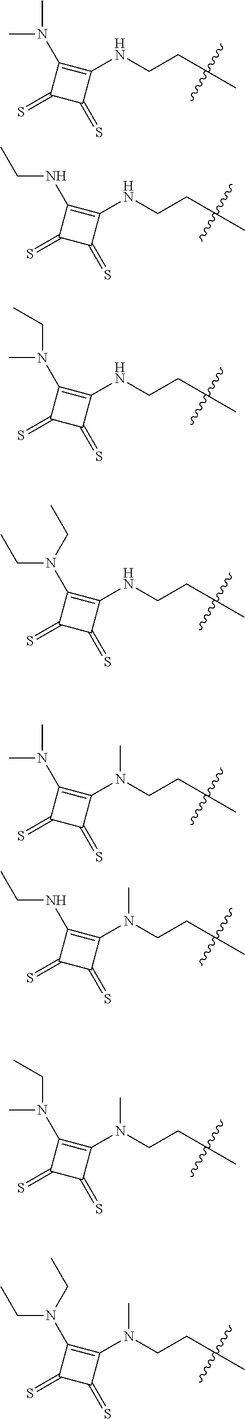

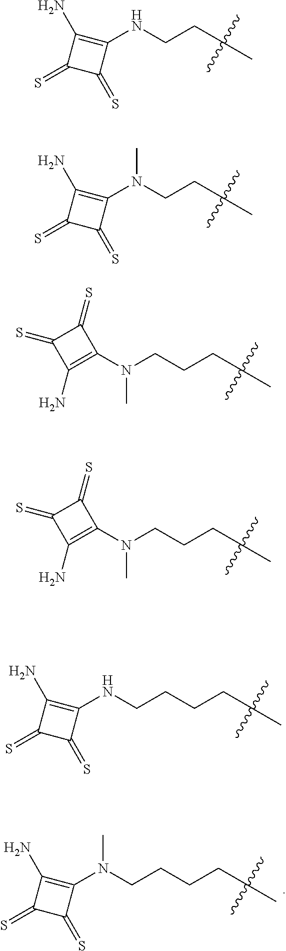

In some embodiments,

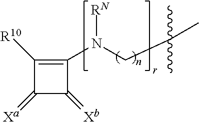

is selected from any of the following groups:

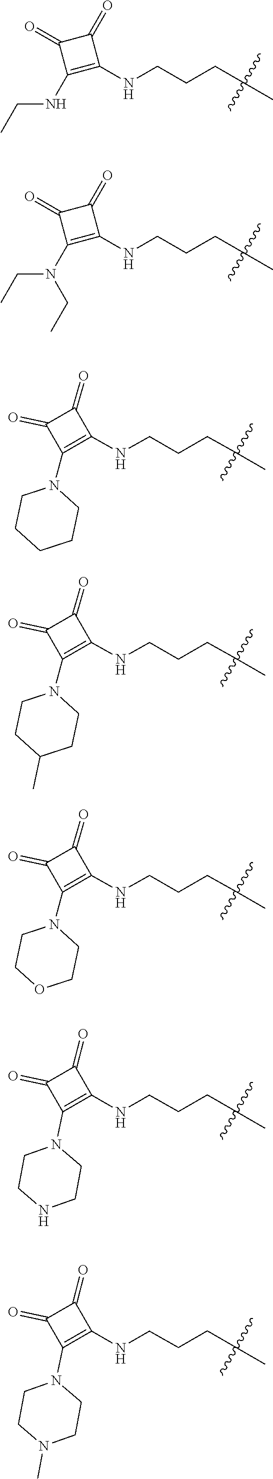

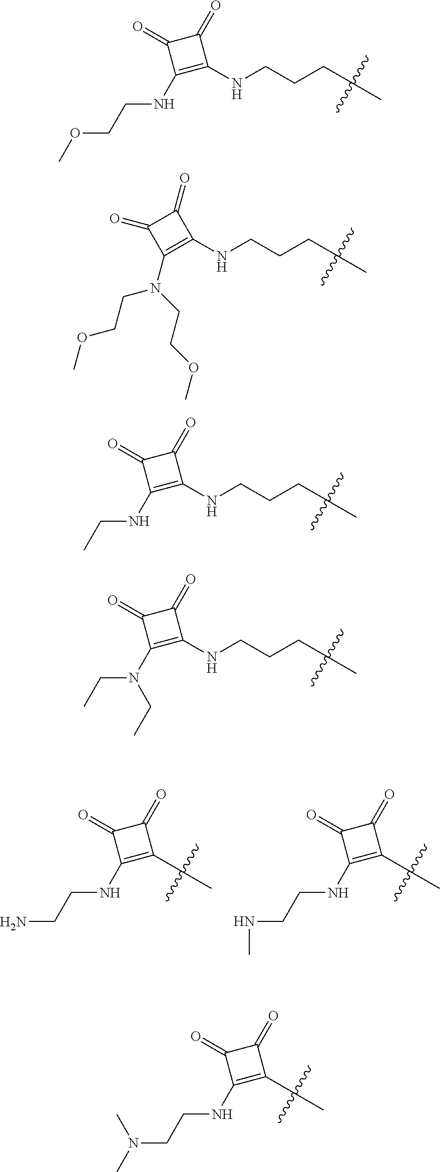

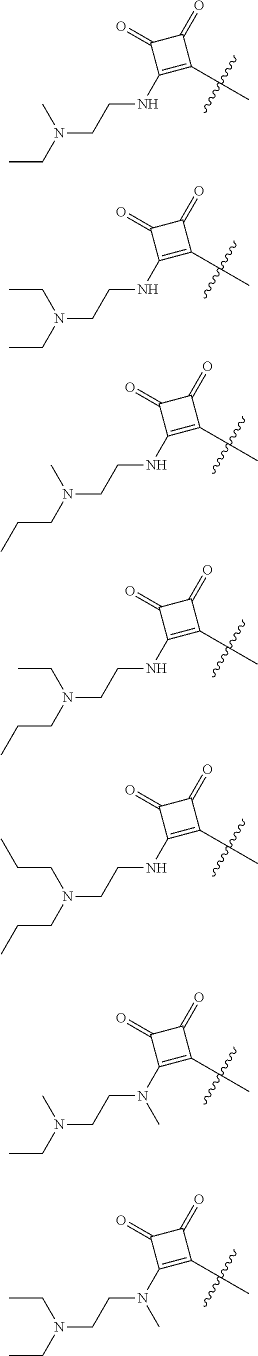

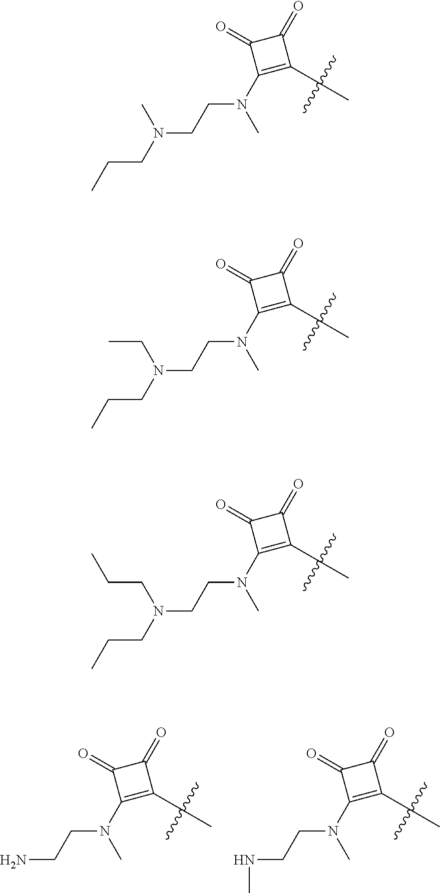 [SMILES: *c1c(N(C)CCN(C)CCC)c(=O)c1=O ; *c1c(N(C)CCN(CC)CCC)c(=O)c1=O ; *c1c(N(C)CCN(CCC)CCC)c(=O)c1=O ; *c1c(N(C)CCN)c(=O)c1=O ; *c1c(N(C)CCNC)c(=O)c1=O]

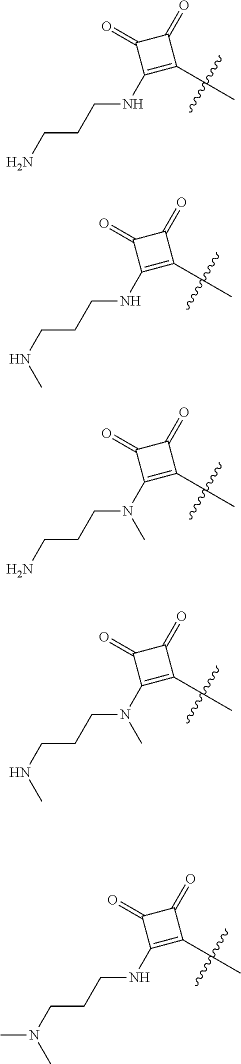 [SMILES: *c1c(NCCCN)c(=O)c1=O ; *c1c(NCCCNC)c(=O)c1=O ; *c1c(N(C)CCCN)c(=O)c1=O ; *c1c(N(C)CCCNC)c(=O)c1=O ; *c1c(NCCCN(C)C)c(=O)c1=O]

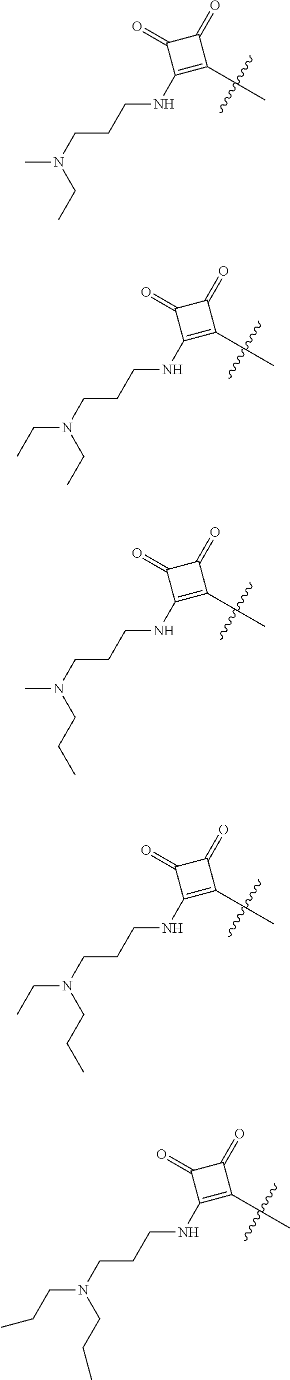 [SMILES: *c1c(NCCCN(C)CC)c(=O)c1=O ; *c1c(NCCCN(CC)CC)c(=O)c1=O ; *c1c(NCCCN(C)CCC)c(=O)c1=O ; *c1c(NCCCN(CC)CCC)c(=O)c1=O ; *c1c(NCCCN(CCC)CCC)c(=O)c1=O]

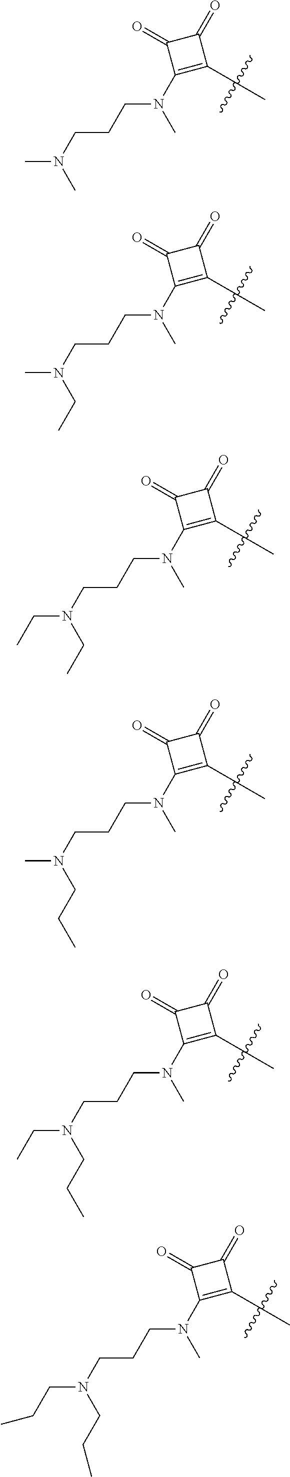 [SMILES: *c1c(N(C)CCCN(C)C)c(=O)c1=O ; *c1c(N(C)CCCN(C)CC)c(=O)c1=O ; *c1c(N(C)CCCN(CC)CC)c(=O)c1=O ; *c1c(N(C)CCCN(C)CCC)c(=O)c1=O ; *c1c(N(C)CCCN(CC)CCC)c(=O)c1=O ; *c1c(N(C)CCCN(CCC)CCC)c(=O)c1=O]

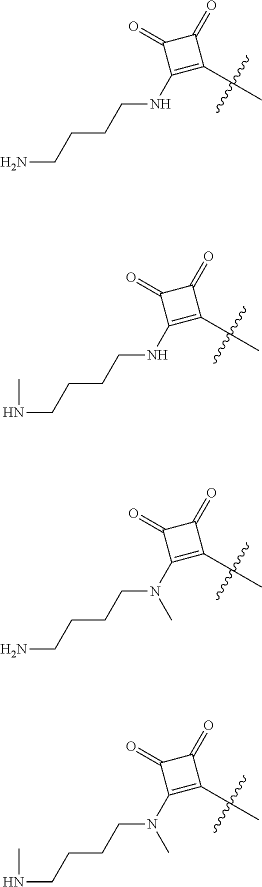 [SMILES: *c1c(NCCCCN)c(=O)c1=O ; *c1c(NCCCCNC)c(=O)c1=O ; *c1c(N(C)CCCCN)c(=O)c1=O ; *c1c(N(C)CCCCNC)c(=O)c1=O]

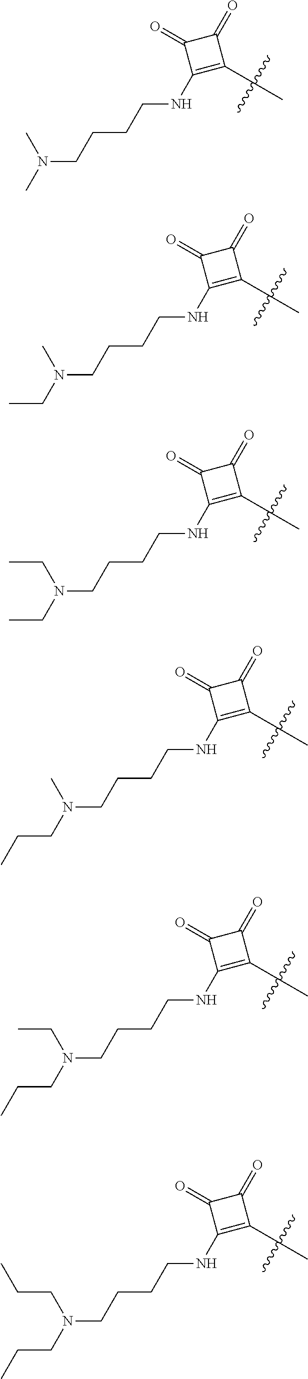 [SMILES: *c1c(NCCCCN(C)C)c(=O)c1=O ; *c1c(NCCCCN(C)CC)c(=O)c1=O ; *c1c(NCCCCN(CC)CC)c(=O)c1=O ; *c1c(NCCCCN(C)CCC)c(=O)c1=O ; *c1c(NCCCCN(CC)CCC)c(=O)c1=O ; *c1c(NCCCCN(CCC)CCC)c(=O)c1=O]

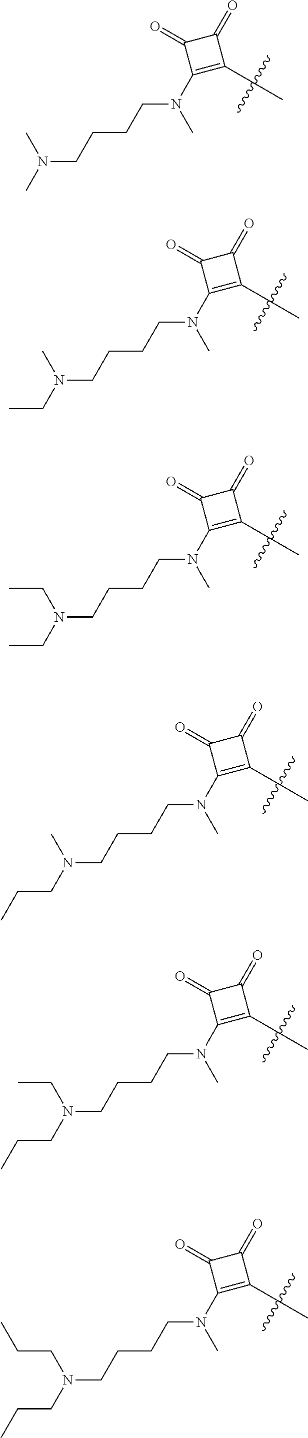 [SMILES: *c1c(N(C)CCCCN(C)C)c(=O)c1=O ; *c1c(N(C)CCCCN(C)CC)c(=O)c1=O ; *c1c(N(C)CCCCN(CC)CC)c(=O)c1=O ; *c1c(N(C)CCCCN(C)CCC)c(=O)c1=O ; *c1c(N(C)CCCCN(CC)CCC)c(=O)c1=O ; *c1c(N(C)CCCCN(CCC)CCC)c(=O)c1=O]

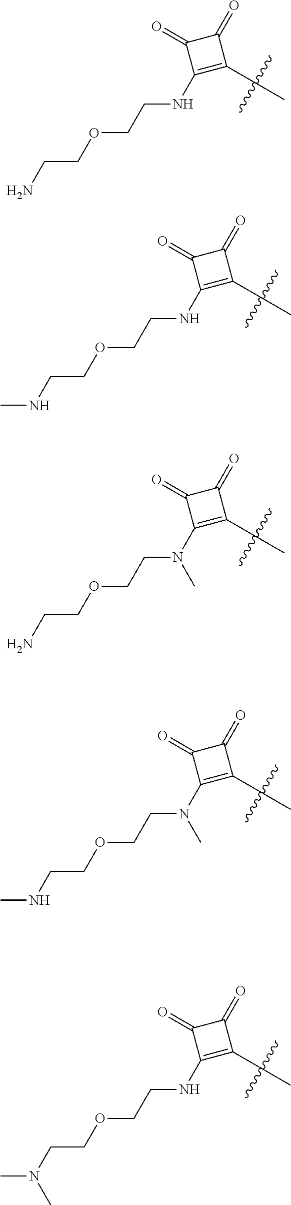 [SMILES: *c1c(NCCOCCN)c(=O)c1=O ; *c1c(NCCOCCNC)c(=O)c1=O ; *c1c(N(C)CCOCCN)c(=O)c1=O ; *c1c(N(C)CCOCCNC)c(=O)c1=O ; *c1c(NCCOCCN(C)C)c(=O)c1=O]

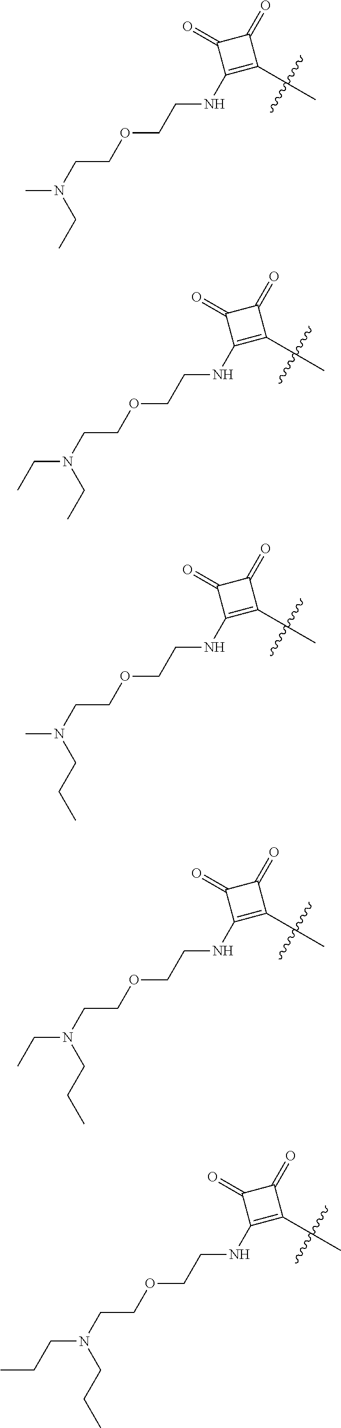 [SMILES: *c1c(NCCOCCN(C)CC)c(=O)c1=O ; *c1c(NCCOCCN(CC)CC)c(=O)c1=O ; *c1c(NCCOCCN(C)CCC)c(=O)c1=O ; *c1c(NCCOCCN(CC)CCC)c(=O)c1=O ; *c1c(NCCOCCN(CCC)CCC)c(=O)c1=O]

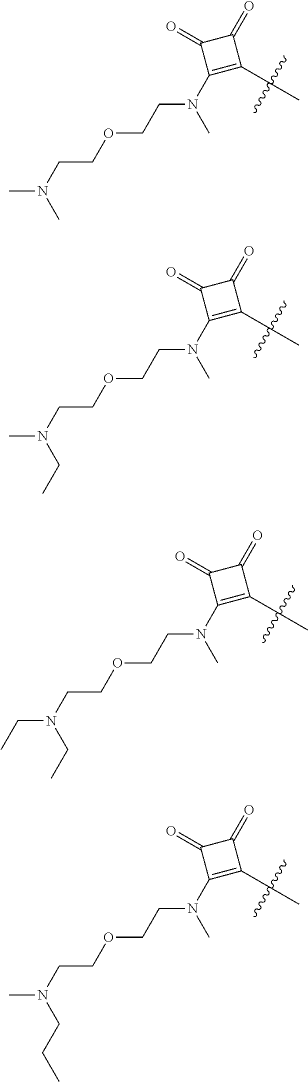 [SMILES: *c1c(N(C)CCOCCN(C)C)c(=O)c1=O ; *c1c(N(C)CCOCCN(C)CC)c(=O)c1=O ; *c1c(N(C)CCOCCN(CC)CC)c(=O)c1=O ; *c1c(N(C)CCOCCN(C)CCC)c(=O)c1=O]

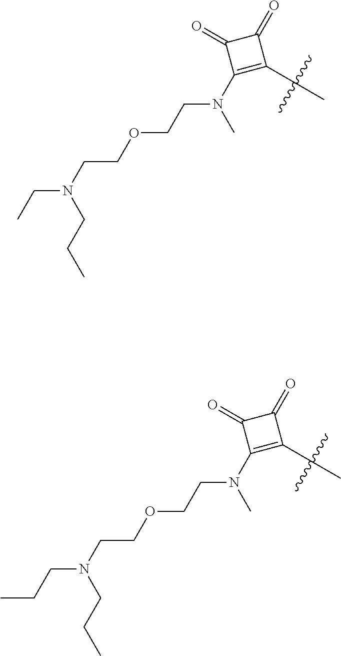 [SMILES: *c1c(N(C)CCOCCN(CC)CCC)c(=O)c1=O ; *c1c(N(C)CCOCCN(CCC)CCC)c(=O)c1=O]

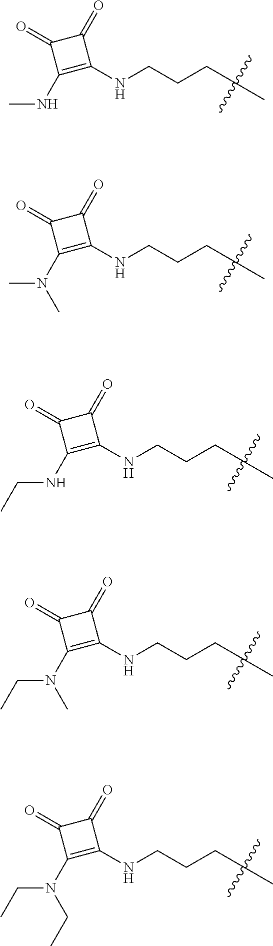 [SMILES: *CCCNc1c(NC)c(=O)c1=O ; *CCCNc1c(N(C)C)c(=O)c1=O ; *CCCNc1c(NCC)c(=O)c1=O ; *CCCNc1c(N(C)CC)c(=O)c1=O ; *CCCNc1c(N(CC)CC)c(=O)c1=O]

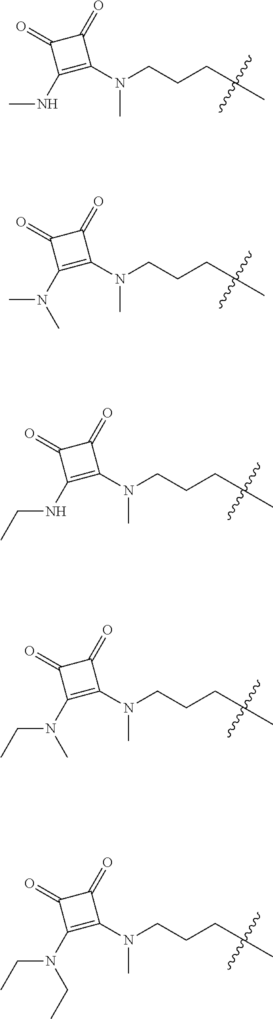 [SMILES: *CCCN(C)c1c(NC)c(=O)c1=O ; *CCCN(C)c1c(N(C)C)c(=O)c1=O ; *CCCN(C)c1c(NCC)c(=O)c1=O ; *CCCN(C)c1c(N(C)CC)c(=O)c1=O ; *CCCN(C)c1c(N(CC)CC)c(=O)c1=O]

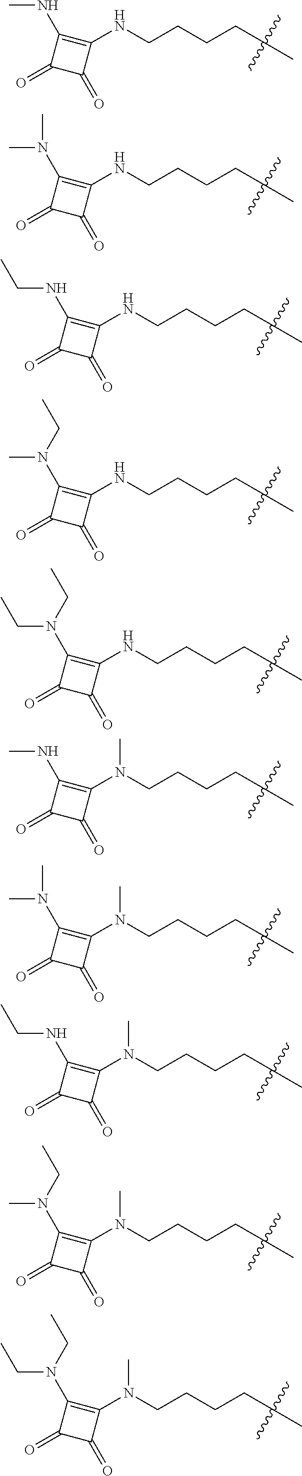

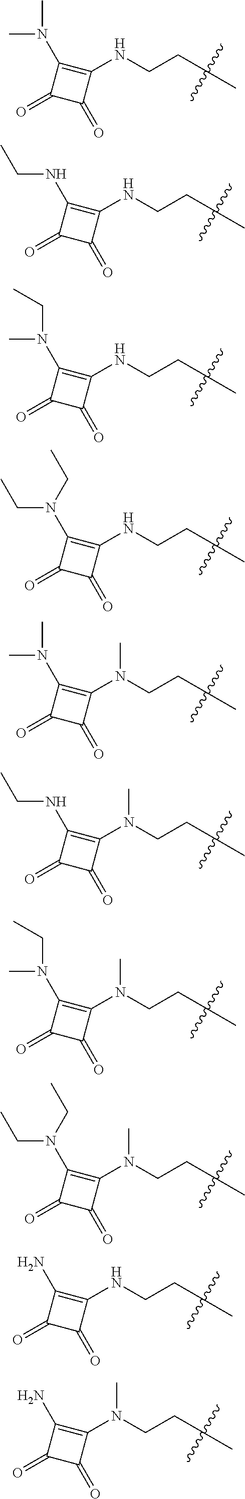

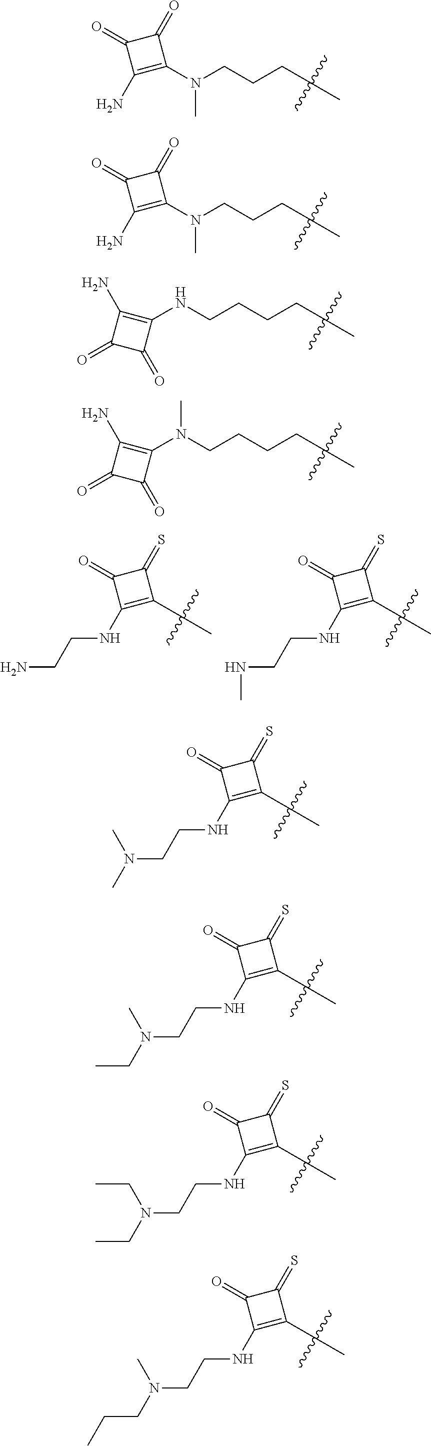

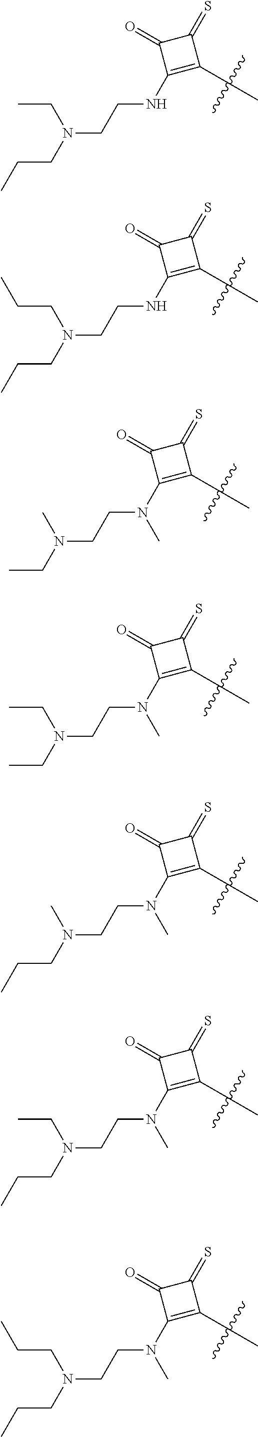

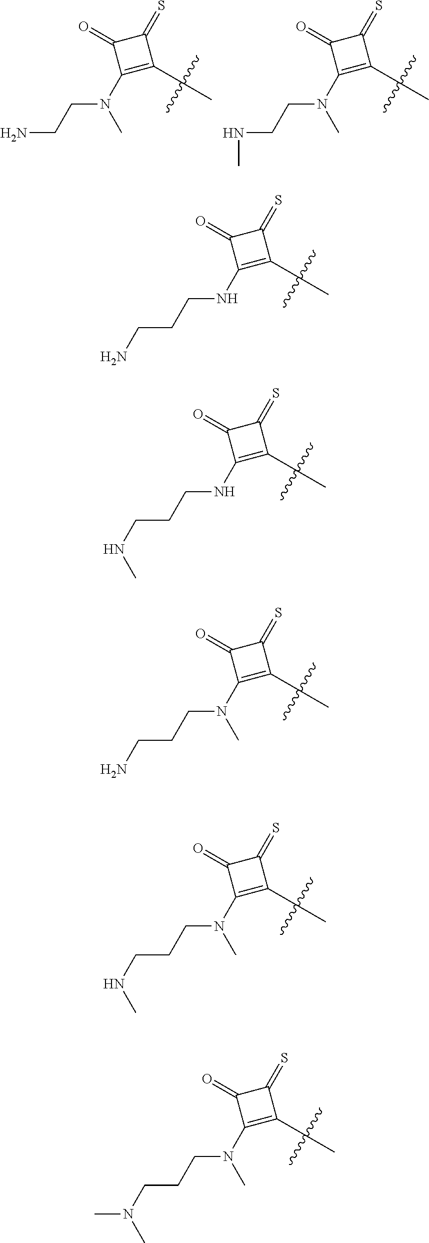

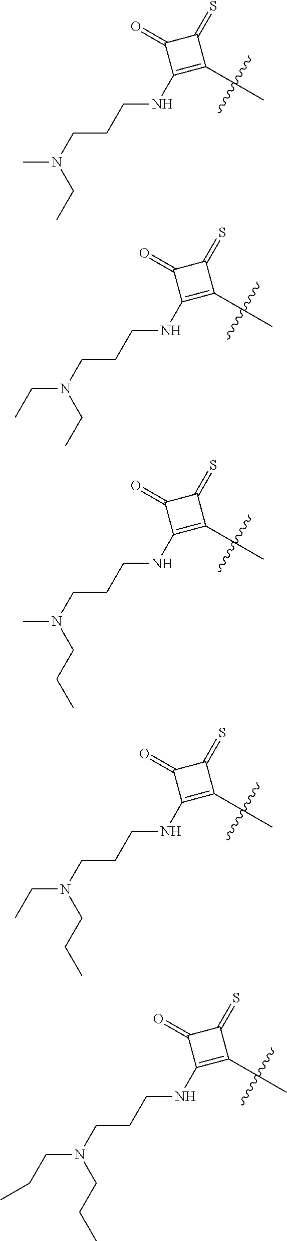 [SMILES: *c1c(NCCCN(C)CC)c(=O)c1=S ; *c1c(NCCCN(CC)CC)c(=O)c1=S ; *c1c(NCCCN(C)CCC)c(=O)c1=S ; *c1c(NCCCN(CC)CCC)c(=O)c1=S ; *c1c(NCCCN(CCC)CCC)c(=O)c1=S]

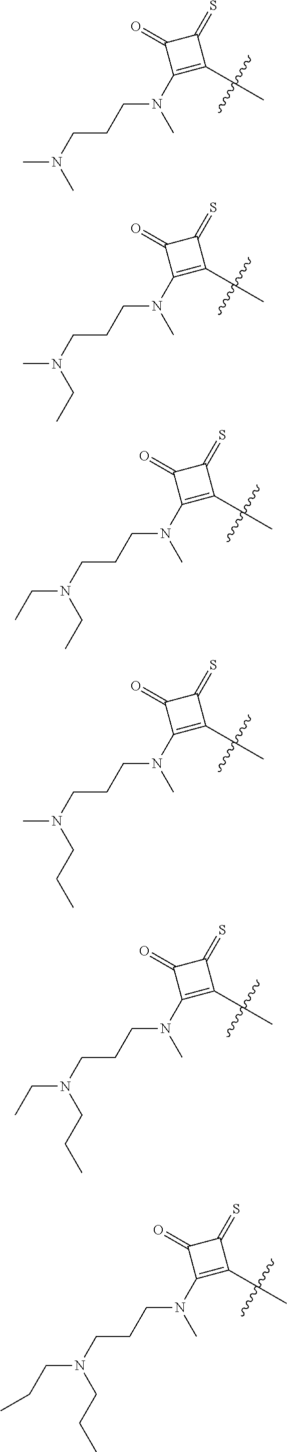 [SMILES: *c1c(N(C)CCCN(C)C)c(=O)c1=S ; *c1c(N(C)CCCN(C)CC)c(=O)c1=S ; *c1c(N(C)CCCN(CC)CC)c(=O)c1=S ; *c1c(N(C)CCCN(C)CCC)c(=O)c1=S ; *c1c(N(C)CCCN(CC)CCC)c(=O)c1=S ; *c1c(N(C)CCCN(CCC)CCC)c(=O)c1=S]

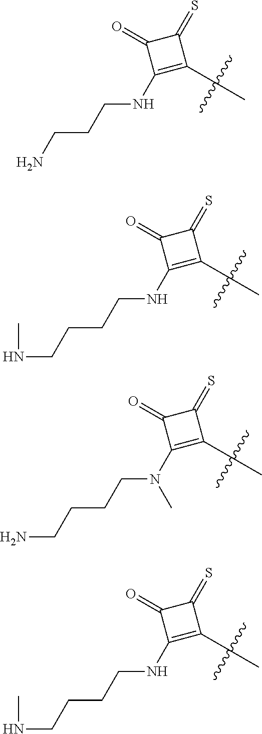 [SMILES: *c1c(NCCCN)c(=O)c1=S ; *c1c(NCCCCNC)c(=O)c1=S ; *c1c(N(C)CCCCN)c(=O)c1=S ; *c1c(NCCCCNC)c(=O)c1=S]

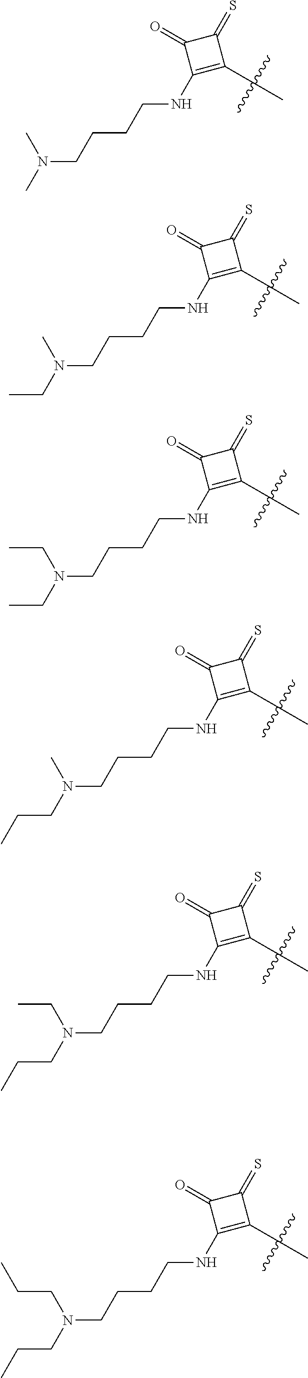 [SMILES: *c1c(NCCCCN(C)C)c(=O)c1=S ; *c1c(NCCCCN(C)CC)c(=O)c1=S ; *c1c(NCCCCN(CC)CC)c(=O)c1=S ; *c1c(NCCCCN(C)CCC)c(=O)c1=S ; *c1c(NCCCCN(CC)CCC)c(=O)c1=S ; *c1c(NCCCCN(CCC)CCC)c(=O)c1=S]

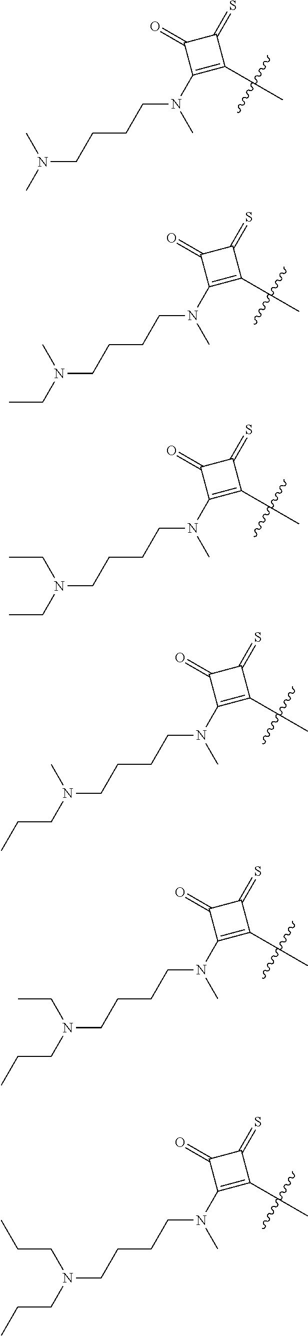 [SMILES: *c1c(N(C)CCCCN(C)C)c(=O)c1=S ; *c1c(N(C)CCCCN(C)CC)c(=O)c1=S ; *c1c(N(C)CCCCN(CC)CC)c(=O)c1=S ; *c1c(N(C)CCCCN(C)CCC)c(=O)c1=S ; *c1c(N(C)CCCCN(CC)CCC)c(=O)c1=S ; *c1c(N(C)CCCCN(CCC)CCC)c(=O)c1=S]

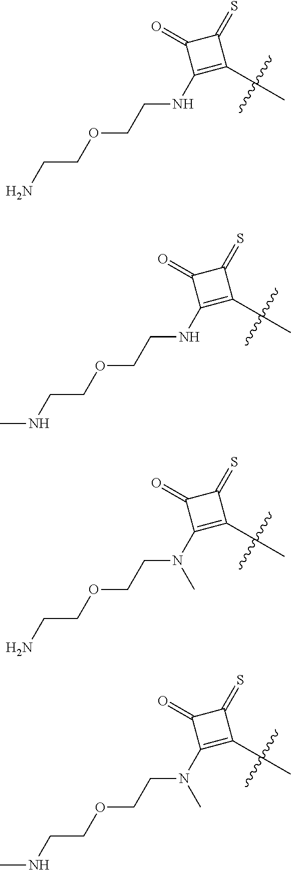 [SMILES: *c1c(NCCOCCN)c(=O)c1=S ; *c1c(NCCOCCNC)c(=O)c1=S ; *c1c(N(C)CCOCCN)c(=O)c1=S ; *c1c(N(C)CCOCCNC)c(=O)c1=S]

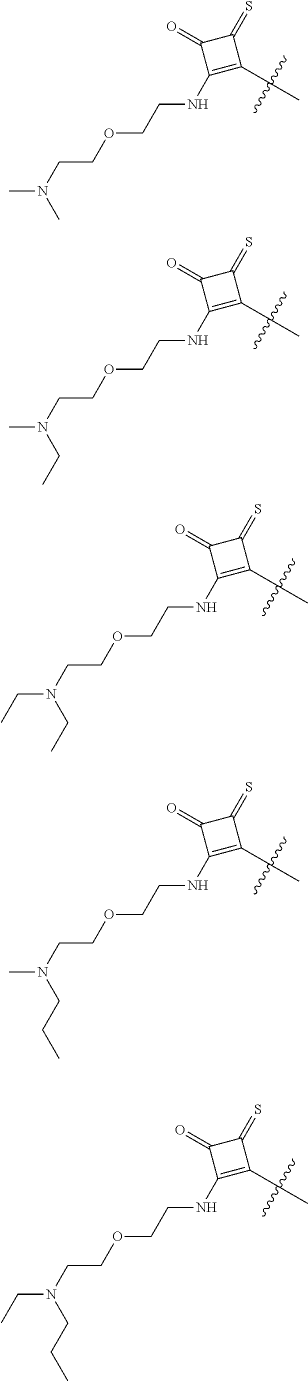 [SMILES: *c1c(NCCOCCN(C)C)c(=O)c1=S ; *c1c(NCCOCCN(C)CC)c(=O)c1=S ; *c1c(NCCOCCN(CC)CC)c(=O)c1=S ; *c1c(NCCOCCN(C)CCC)c(=O)c1=S ; *c1c(NCCOCCN(CC)CCC)c(=O)c1=S]

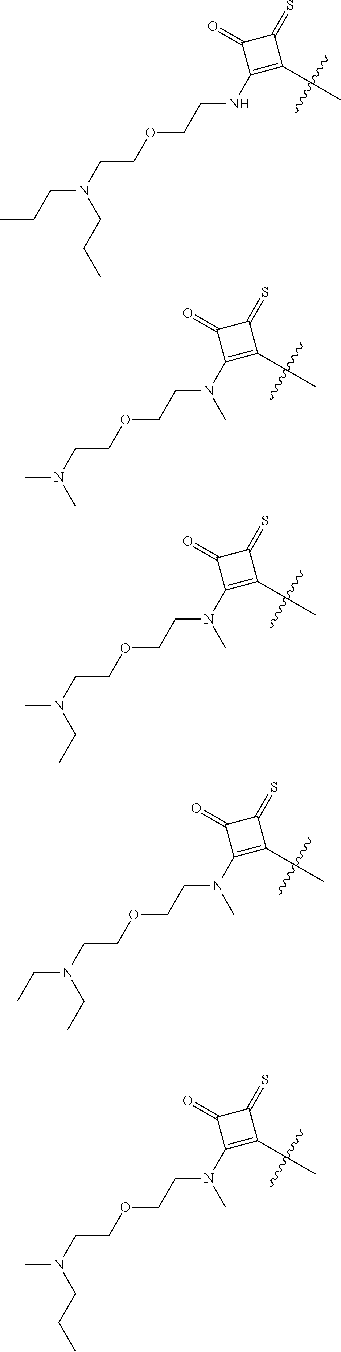 [SMILES: *c1c(NCCOCCN(CCC)CCC)c(=O)c1=S ; *c1c(N(C)CCOCCN(C)C)c(=O)c1=S ; *c1c(N(C)CCOCCN(C)CC)c(=O)c1=S ; *c1c(N(C)CCOCCN(CC)CC)c(=O)c1=S ; *c1c(N(C)CCOCCN(C)CCC)c(=O)c1=S]

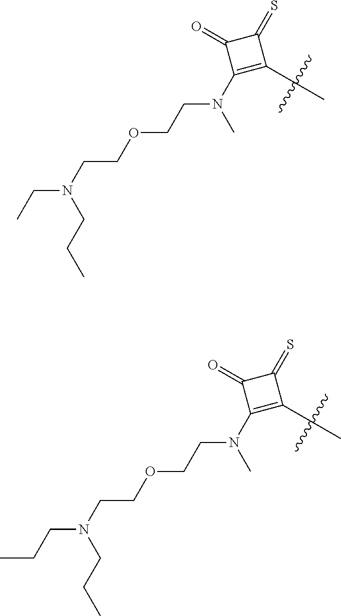 [SMILES: *c1c(N(C)CCOCCN(CC)CCC)c(=O)c1=S ; *c1c(N(C)CCOCCN(CCC)CCC)c(=O)c1=S]

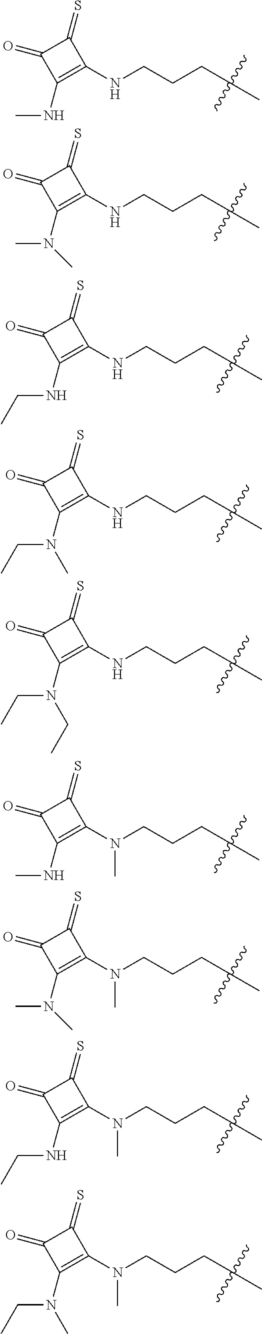

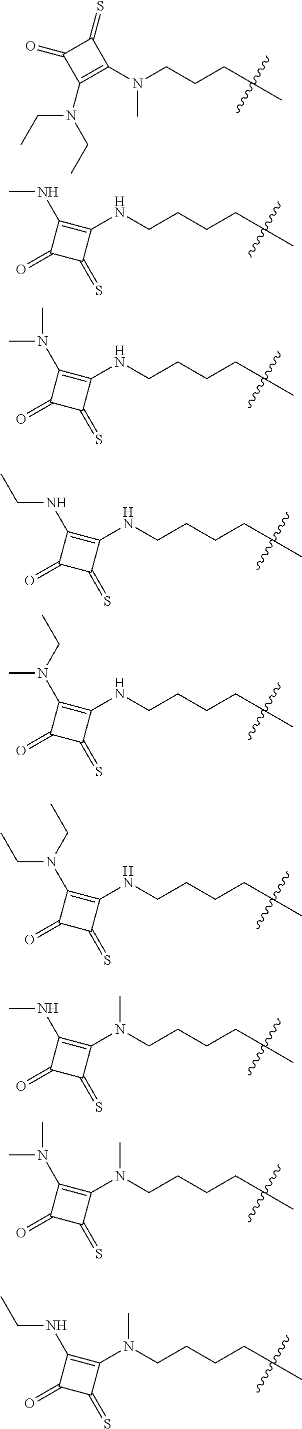

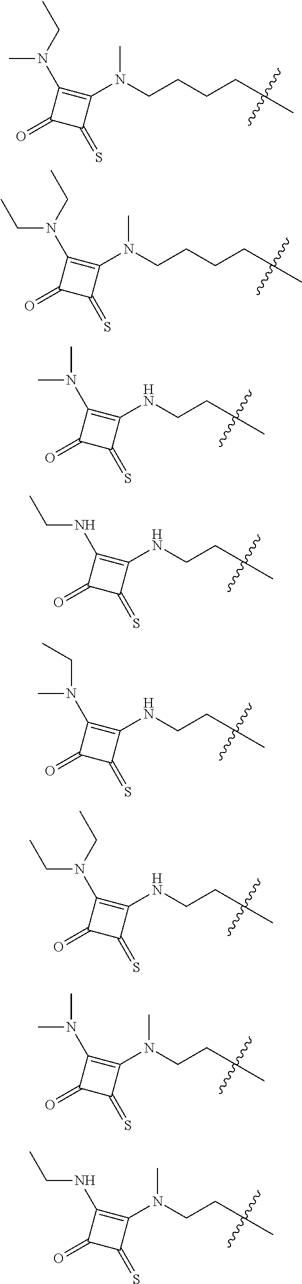

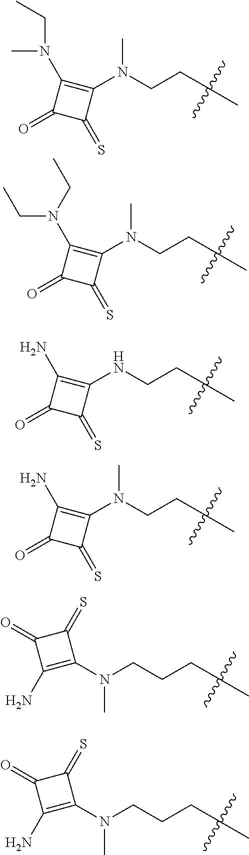

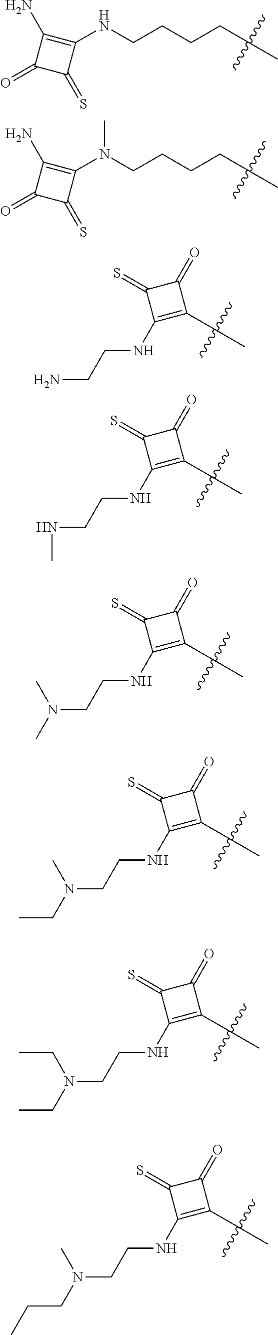

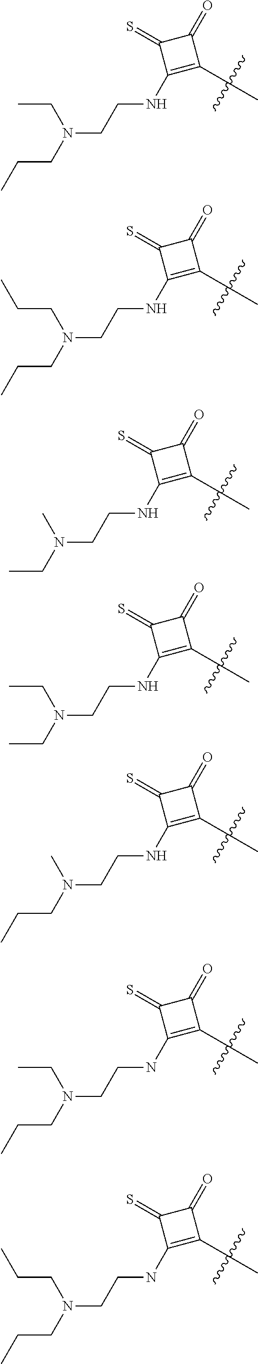

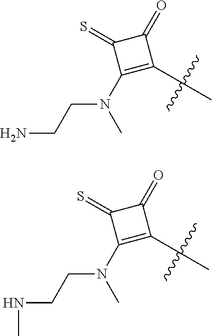 [SMILES: *c1c(N(C)CCN)c(=S)c1=O ; *c1c(N(C)CCNC)c(=S)c1=O]

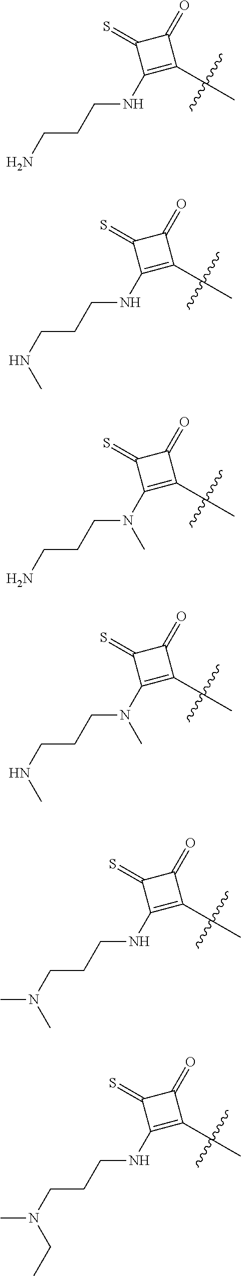 [SMILES: *c1c(NCCCN)c(=S)c1=O ; *c1c(NCCCNC)c(=S)c1=O ; *c1c(N(C)CCCN)c(=S)c1=O ; *c1c(N(C)CCCNC)c(=S)c1=O ; *c1c(NCCCN(C)C)c(=S)c1=O ; *c1c(NCCCN(C)CC)c(=S)c1=O]

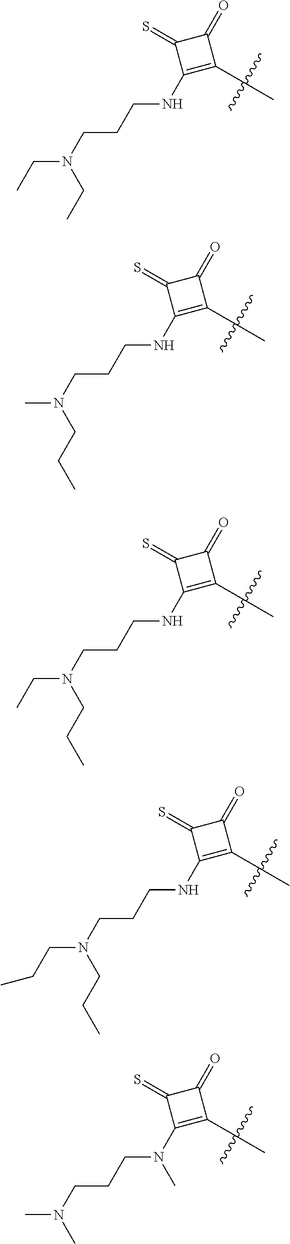 [SMILES: *c1c(NCCCN(CC)CC)c(=S)c1=O ; *c1c(NCCCN(C)CCC)c(=S)c1=O ; *c1c(NCCCN(CC)CCC)c(=S)c1=O ; *c1c(NCCCN(CCC)CCC)c(=S)c1=O ; *c1c(N(C)CCCN(C)C)c(=S)c1=O]

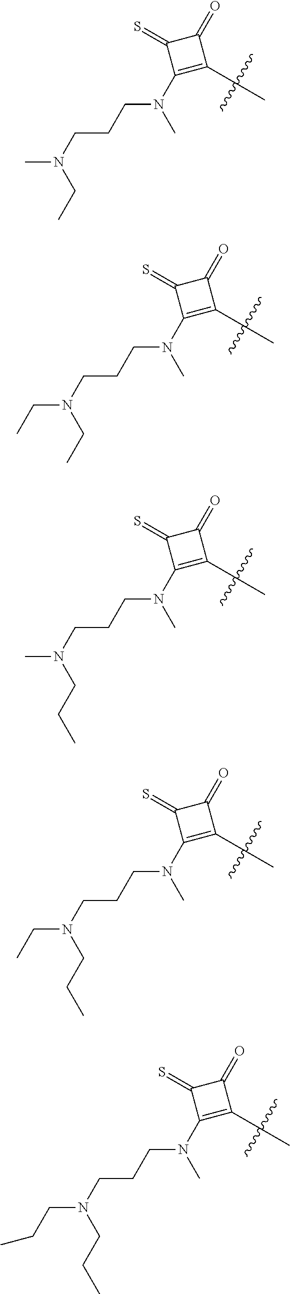 [SMILES: *c1c(N(C)CCCN(C)CC)c(=S)c1=O ; *c1c(N(C)CCCN(CC)CC)c(=S)c1=O ; *c1c(N(C)CCCN(C)CCC)c(=S)c1=O ; *c1c(N(C)CCCN(CC)CCC)c(=S)c1=O ; *c1c(N(C)CCCN(CCC)CCC)c(=S)c1=O]

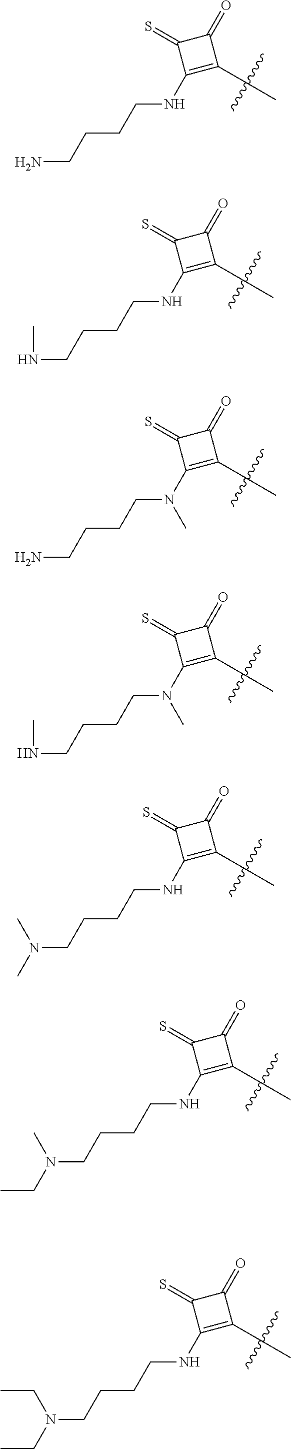

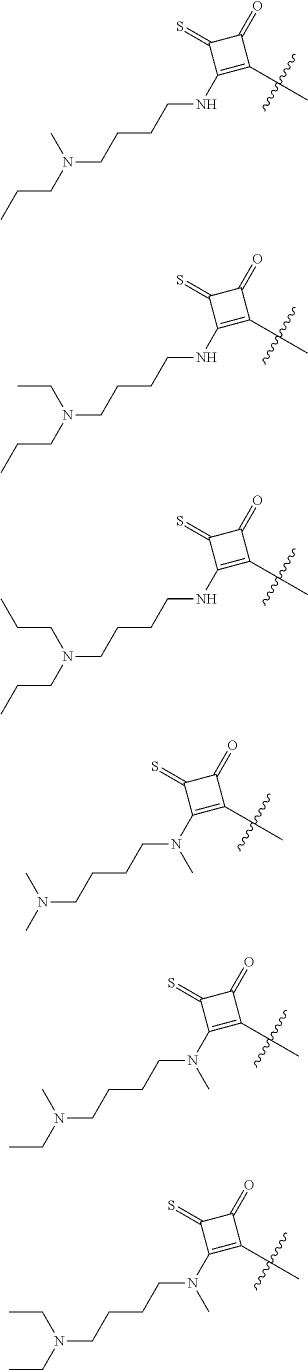 [SMILES: *c1c(NCCCCN(C)CCC)c(=S)c1=O ; *c1c(NCCCCN(CC)CCC)c(=S)c1=O ; *c1c(NCCCCN(CCC)CCC)c(=S)c1=O ; *c1c(N(C)CCCCN(C)C)c(=S)c1=O ; *c1c(N(C)CCCCN(C)CC)c(=S)c1=O ; *c1c(N(C)CCCCN(CC)CC)c(=S)c1=O]

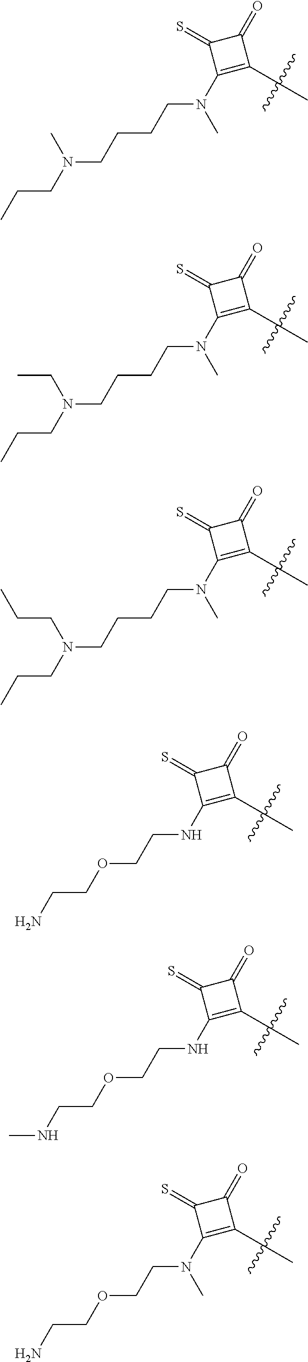 [SMILES: *c1c(N(C)CCCCN(C)CCC)c(=S)c1=O ; *c1c(N(C)CCCCN(CC)CCC)c(=S)c1=O ; *c1c(N(C)CCCCN(CCC)CCC)c(=S)c1=O ; *c1c(NCCOCCN)c(=S)c1=O ; *c1c(NCCOCCNC)c(=S)c1=O ; *c1c(N(C)CCOCCN)c(=S)c1=O]

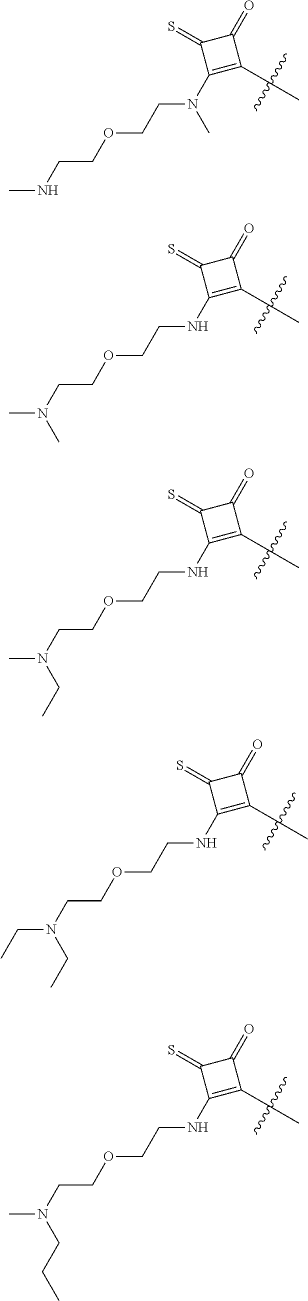 [SMILES: *c1c(N(C)CCOCCNC)c(=S)c1=O ; *c1c(NCCOCCN(C)C)c(=S)c1=O ; *c1c(NCCOCCN(C)CC)c(=S)c1=O ; *c1c(NCCOCCN(CC)CC)c(=S)c1=O ; *c1c(NCCOCCN(C)CCC)c(=S)c1=O]

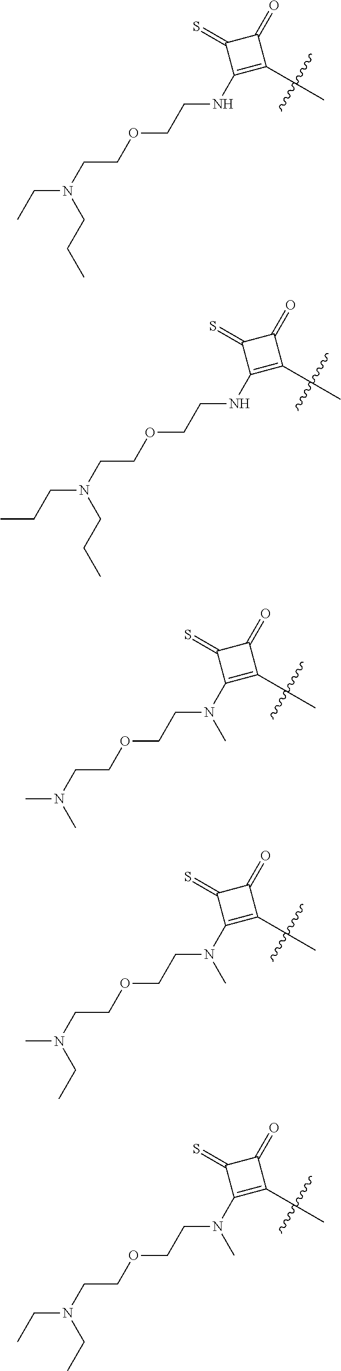 [SMILES: *c1c(NCCOCCN(CC)CCC)c(=S)c1=O ; *c1c(NCCOCCN(CCC)CCC)c(=S)c1=O ; *c1c(N(C)CCOCCN(C)C)c(=S)c1=O ; *c1c(N(C)CCOCCN(C)CC)c(=S)c1=O ; *c1c(N(C)CCOCCN(CC)CC)c(=S)c1=O]

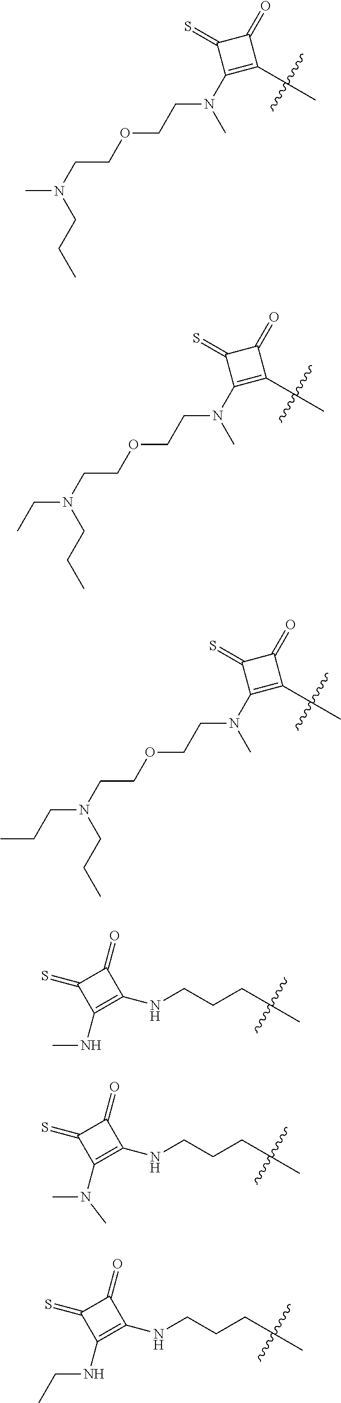

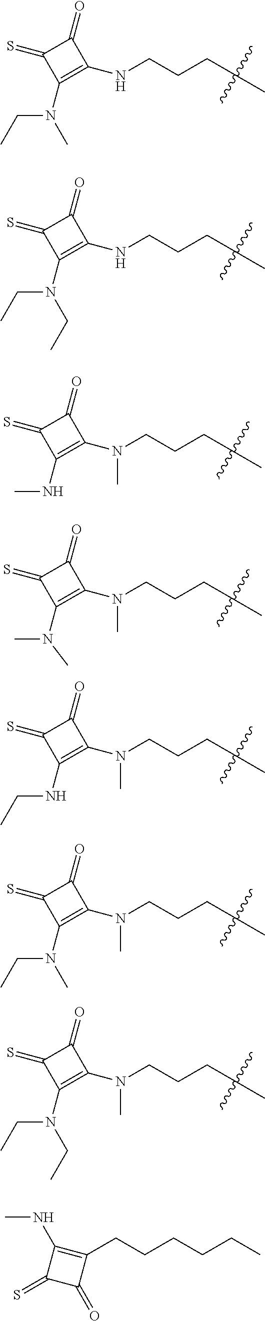

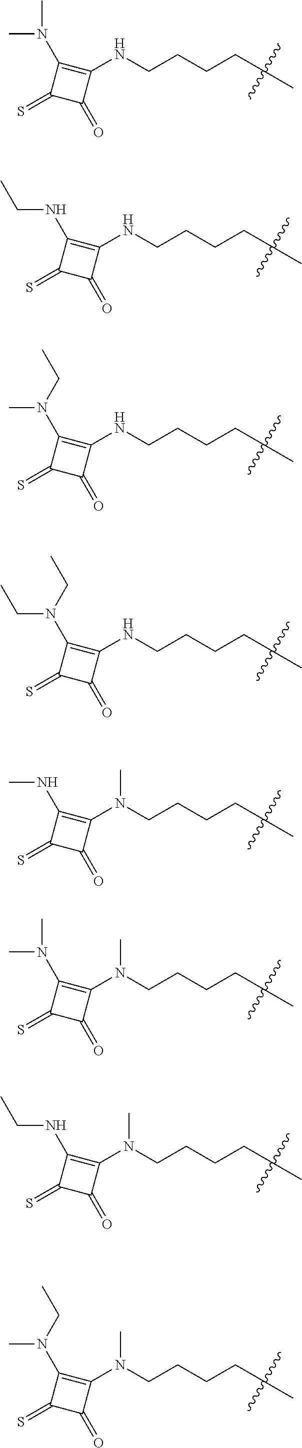

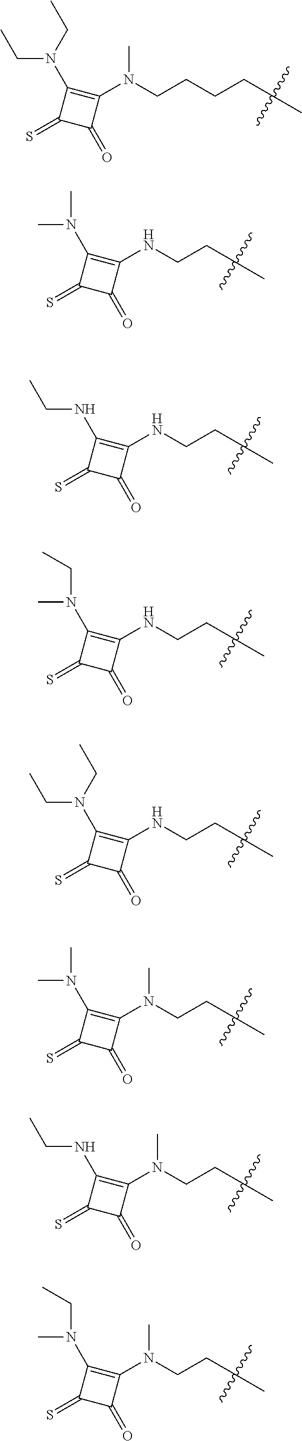

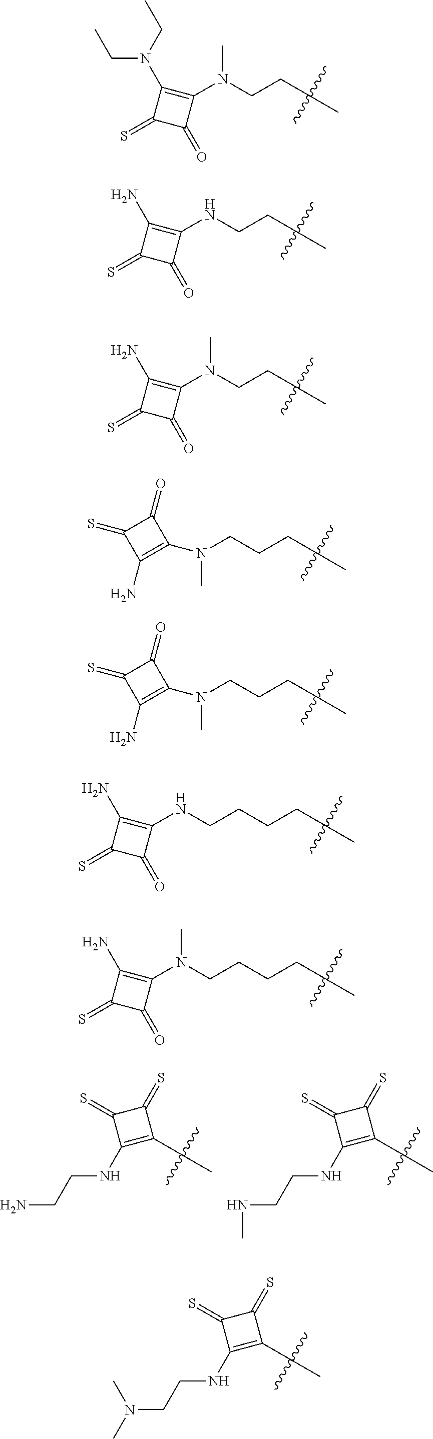

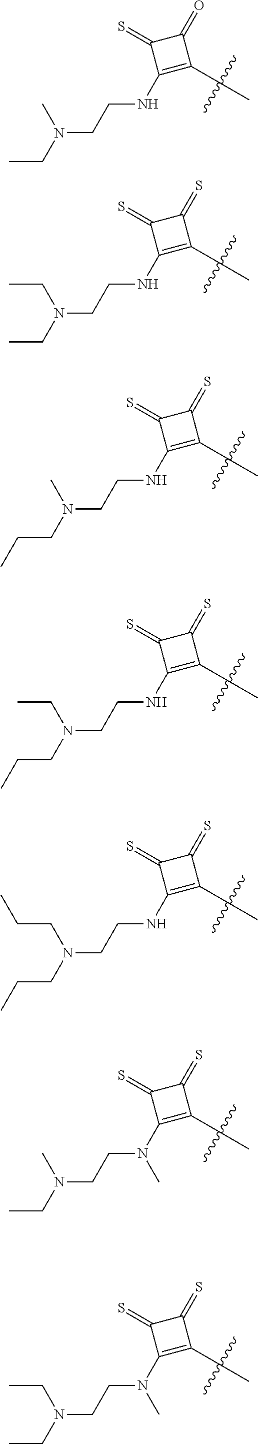

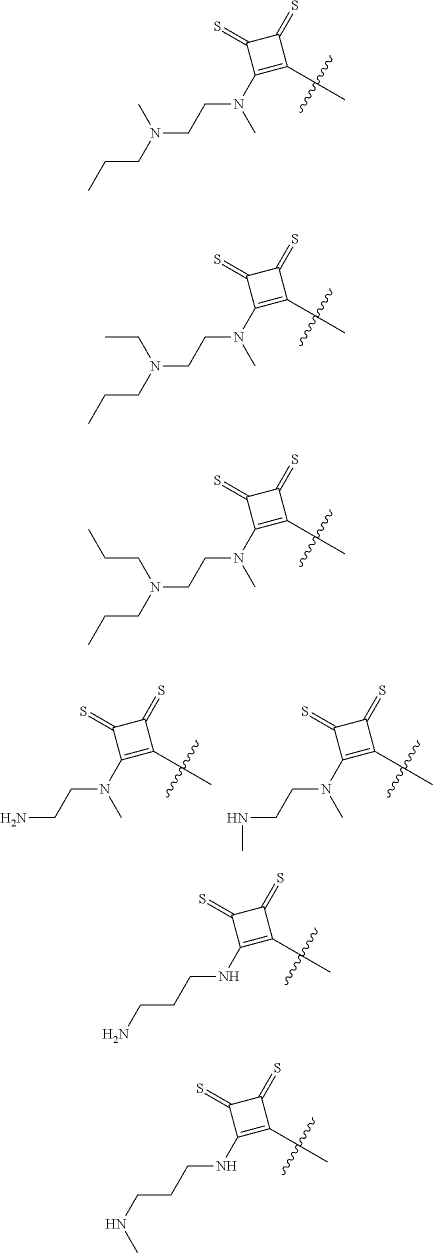

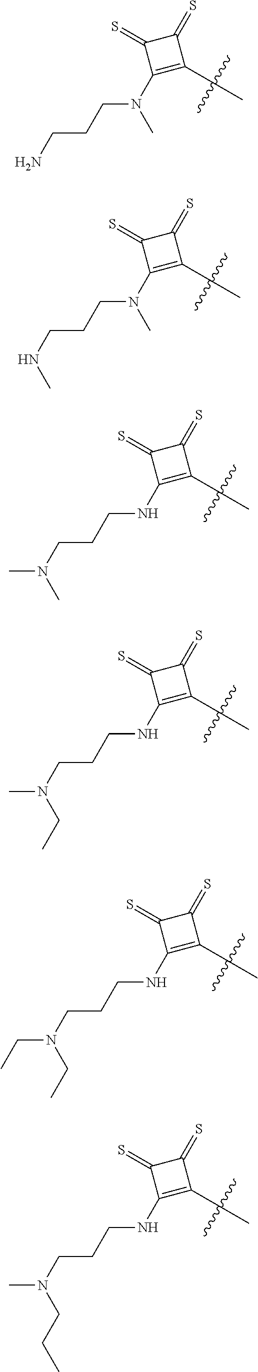 [SMILES: *c1c(N(C)CCCN)c(=S)c1=S ; *c1c(N(C)CCCNC)c(=S)c1=S ; *c1c(NCCCN(C)C)c(=S)c1=S ; *c1c(NCCCN(C)CC)c(=S)c1=S ; *c1c(NCCCN(CC)CC)c(=S)c1=S ; *c1c(NCCCN(C)CCC)c(=S)c1=S]

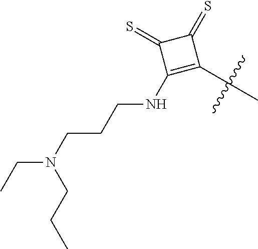 [SMILES: *c1c(NCCCN(CC)CCC)c(=S)c1=S]

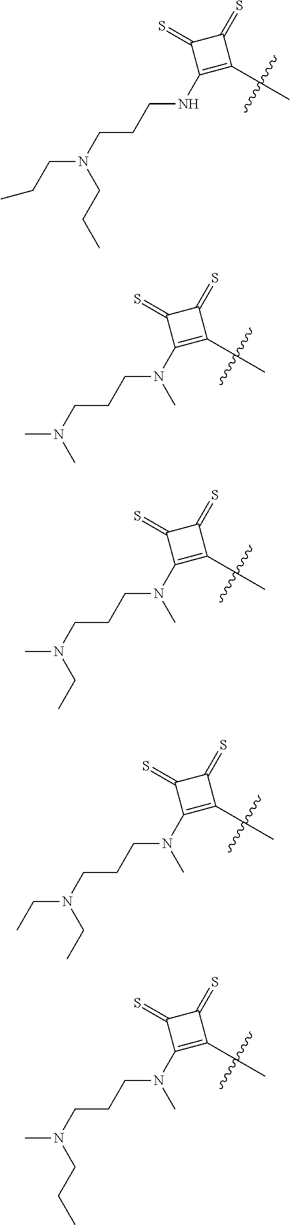 [SMILES: *c1c(NCCCN(CCC)CCC)c(=S)c1=S ; *c1c(N(C)CCCN(C)C)c(=S)c1=S ; *c1c(N(C)CCCN(C)CC)c(=S)c1=S ; *c1c(N(C)CCCN(CC)CC)c(=S)c1=S ; *c1c(N(C)CCCN(C)CCC)c(=S)c1=S]

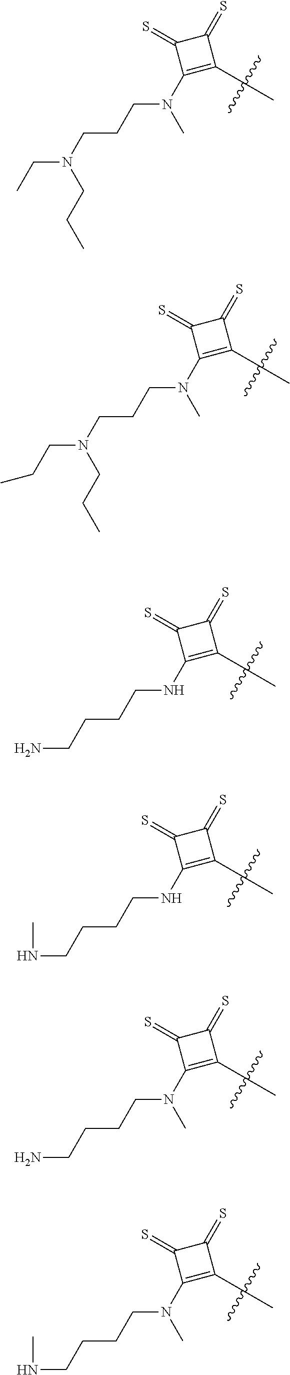 [SMILES: *c1c(N(C)CCCN(CC)CCC)c(=S)c1=S ; *c1c(N(C)CCCN(CCC)CCC)c(=S)c1=S ; *c1c(NCCCCN)c(=S)c1=S ; *c1c(NCCCCNC)c(=S)c1=S ; *c1c(N(C)CCCCN)c(=S)c1=S ; *c1c(N(C)CCCCNC)c(=S)c1=S]

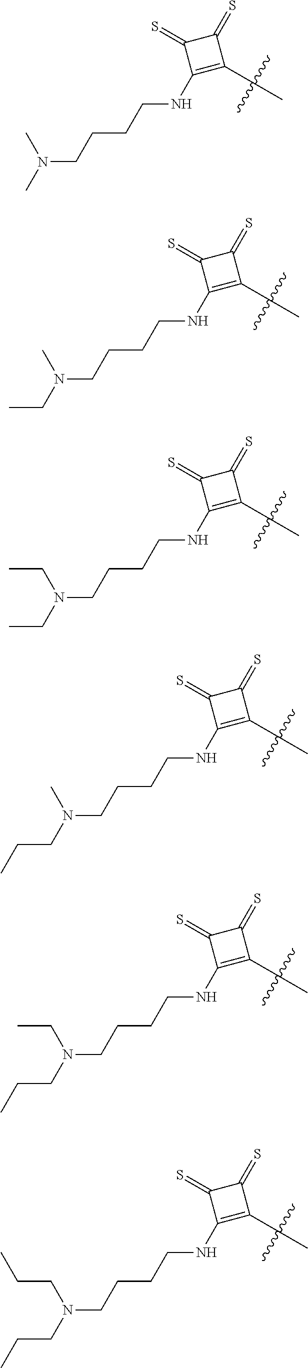 [SMILES: *c1c(NCCCCN(C)C)c(=S)c1=S ; *c1c(NCCCCN(C)CC)c(=S)c1=S ; *c1c(NCCCCN(CC)CC)c(=S)c1=S ; *c1c(NCCCCN(C)CCC)c(=S)c1=S ; *c1c(NCCCCN(CC)CCC)c(=S)c1=S ; *c1c(NCCCCN(CCC)CCC)c(=S)c1=S]

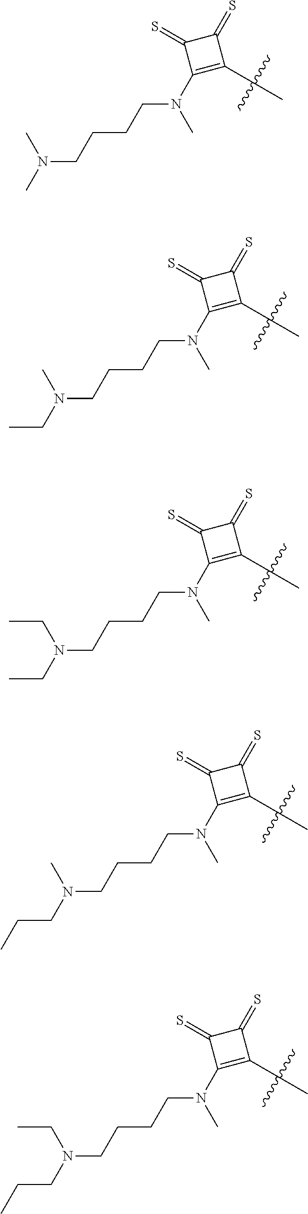 [SMILES: *c1c(N(C)CCCCN(C)C)c(=S)c1=S ; *c1c(N(C)CCCCN(C)CC)c(=S)c1=S ; *c1c(N(C)CCCCN(CC)CC)c(=S)c1=S ; *c1c(N(C)CCCCN(C)CCC)c(=S)c1=S ; *c1c(N(C)CCCCN(CC)CCC)c(=S)c1=S]

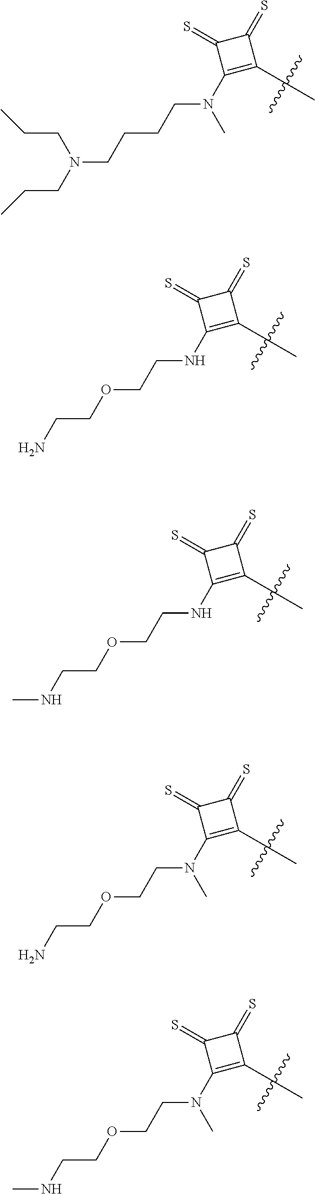 [SMILES: *c1c(N(C)CCCCN(CCC)CCC)c(=S)c1=S ; *c1c(NCCOCCN)c(=S)c1=S ; *c1c(NCCOCCNC)c(=S)c1=S ; *c1c(N(C)CCOCCN)c(=S)c1=S ; *c1c(N(C)CCOCCNC)c(=S)c1=S]

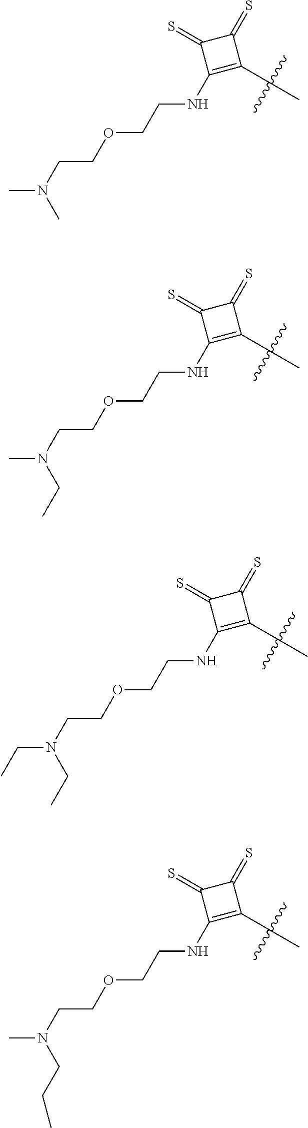 [SMILES: *c1c(NCCOCCN(C)C)c(=S)c1=S ; *c1c(NCCOCCN(C)CC)c(=S)c1=S ; *c1c(NCCOCCN(CC)CC)c(=S)c1=S ; *c1c(NCCOCCN(C)CCC)c(=S)c1=S]

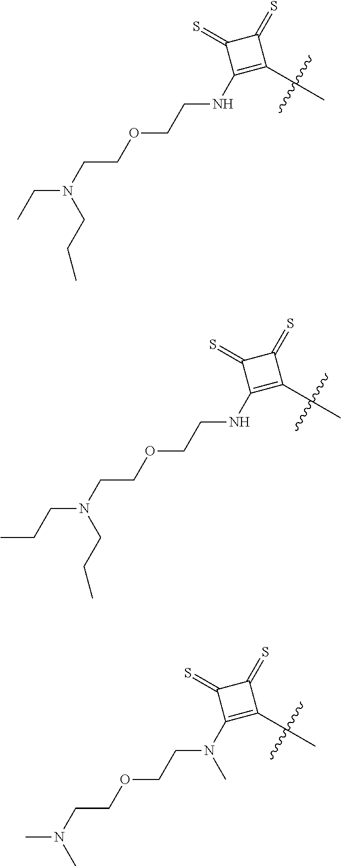 [SMILES: *c1c(NCCOCCN(CC)CCC)c(=S)c1=S ; *c1c(NCCOCCN(CCC)CCC)c(=S)c1=S ; *c1c(N(C)CCOCCN(C)C)c(=S)c1=S]

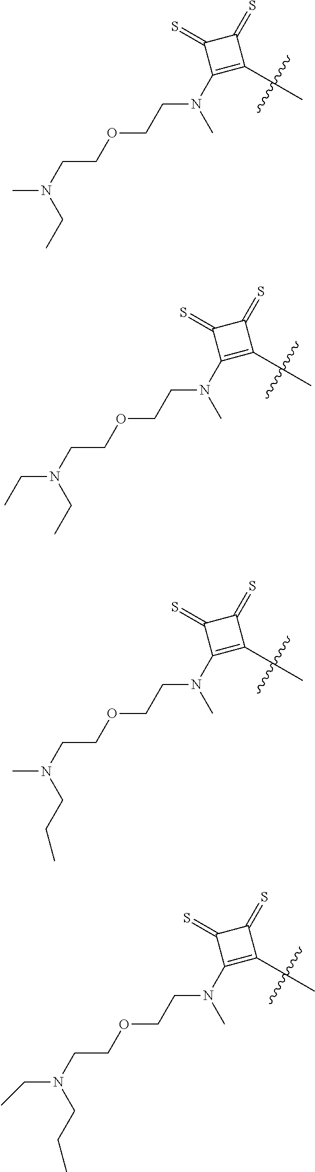 [SMILES: *c1c(N(C)CCOCCN(C)CC)c(=S)c1=S ; *c1c(N(C)CCOCCN(CC)CC)c(=S)c1=S ; *c1c(N(C)CCOCCN(C)CCC)c(=S)c1=S ; *c1c(N(C)CCOCCN(CC)CCC)c(=S)c1=S]

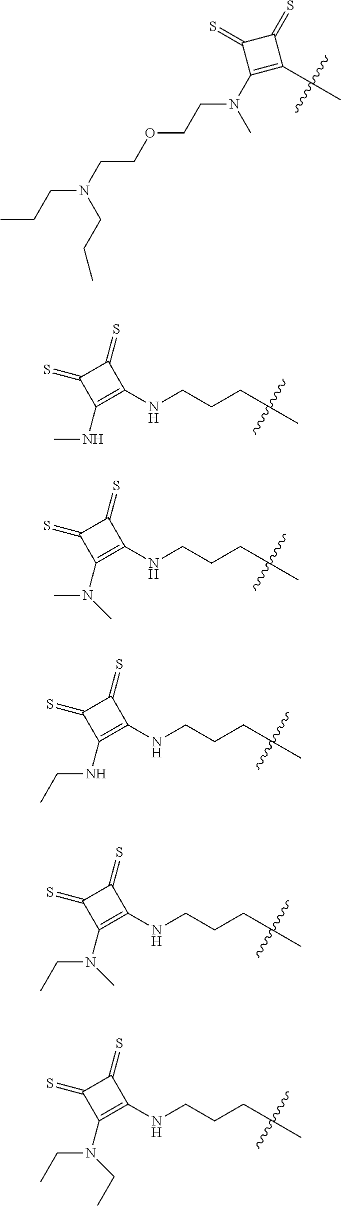

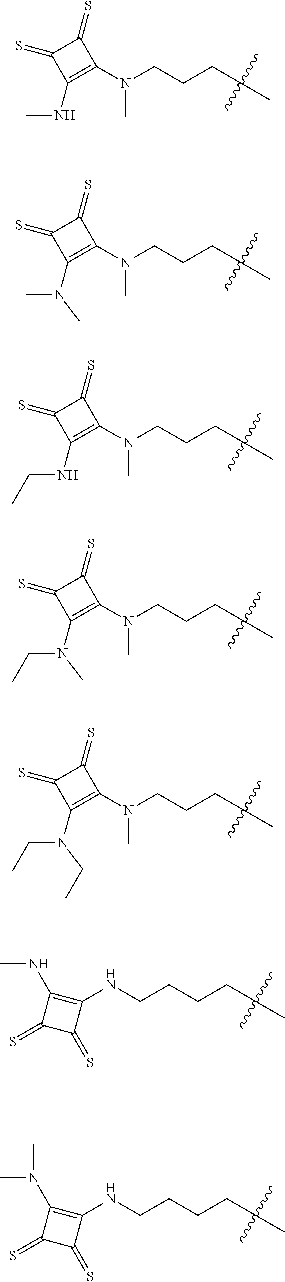

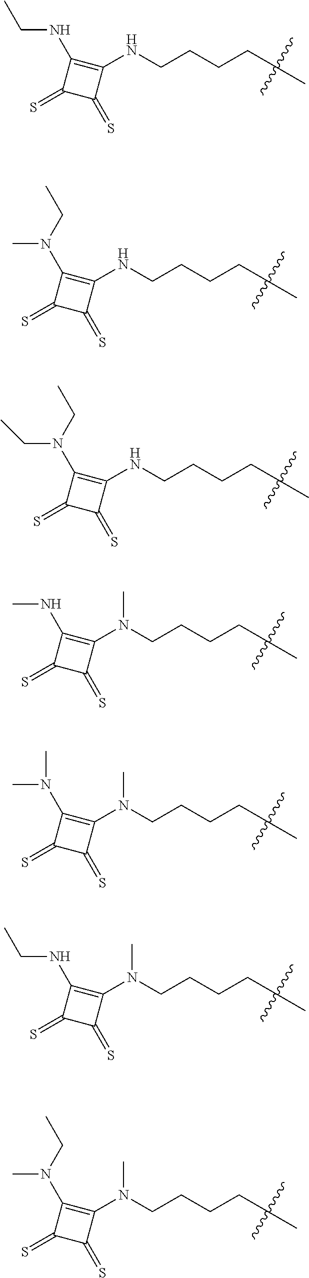

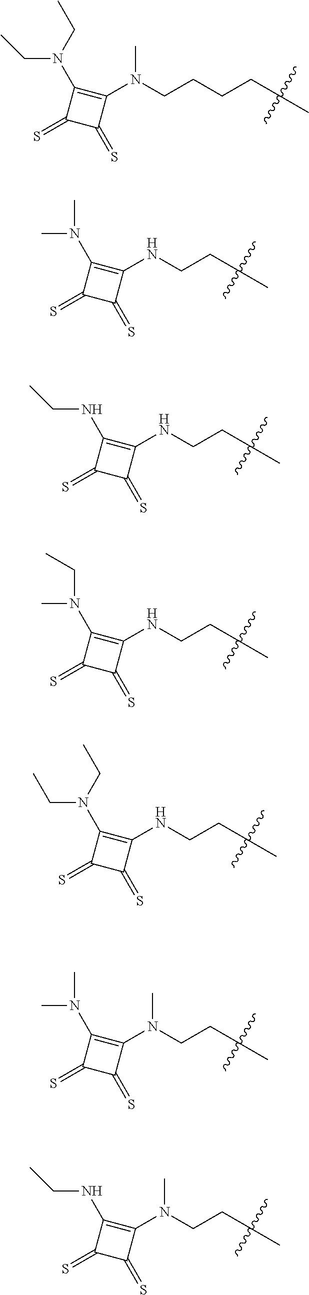

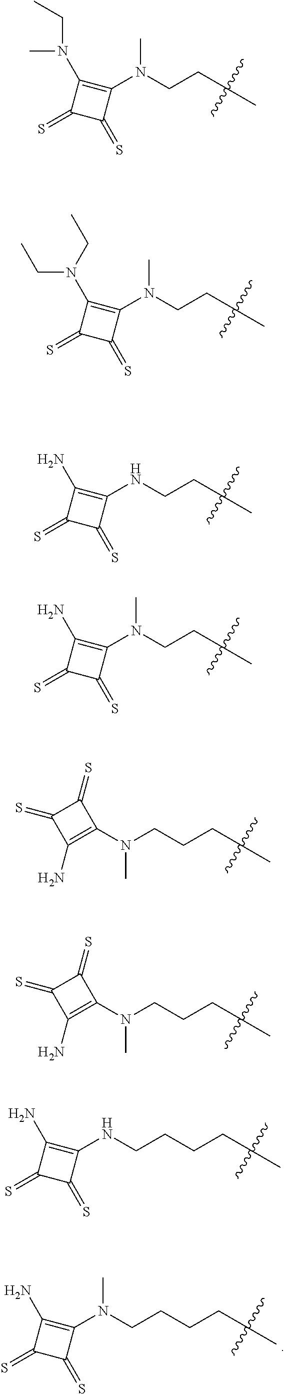

In some embodiments, R⁴ is selected from any of the following groups:

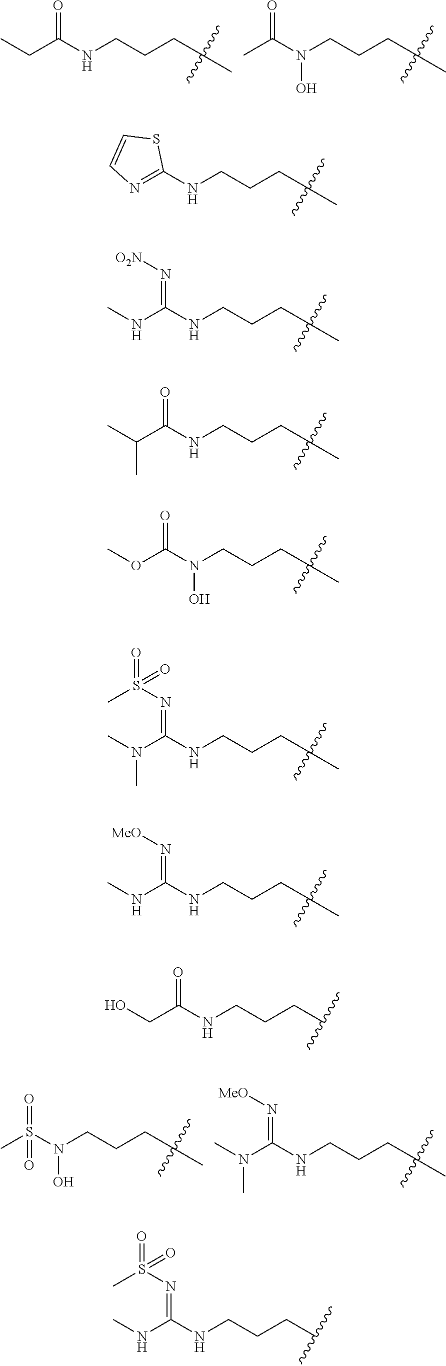

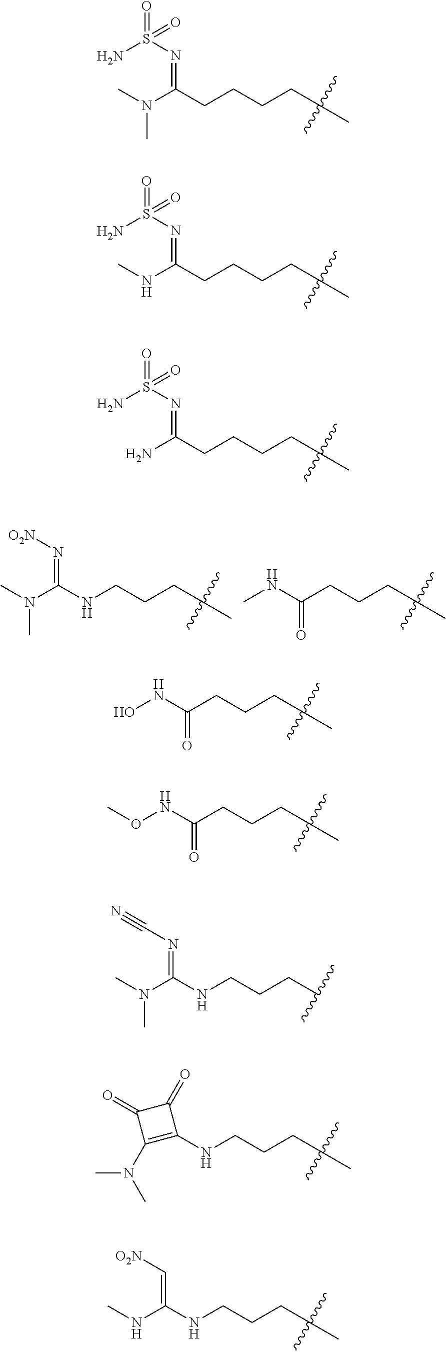

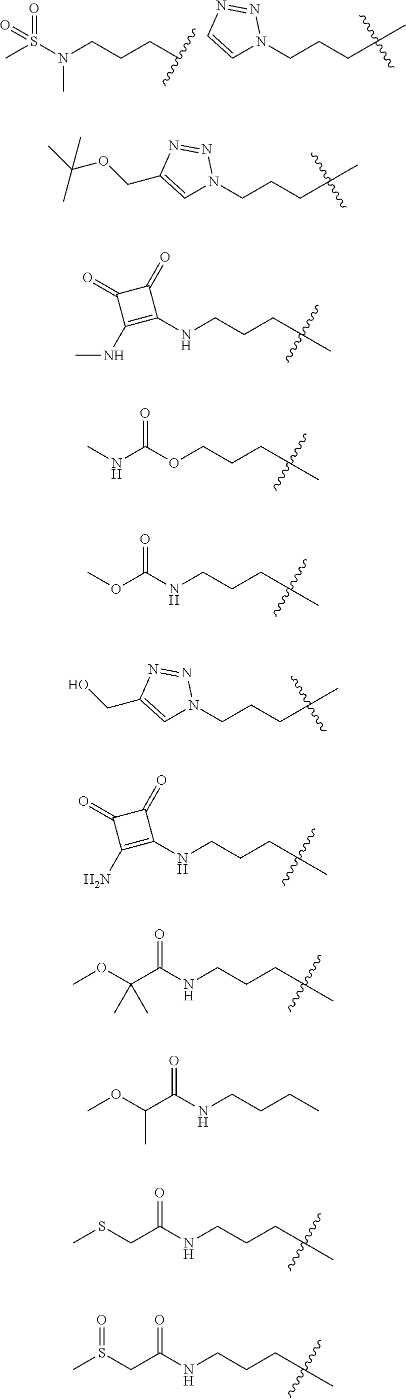

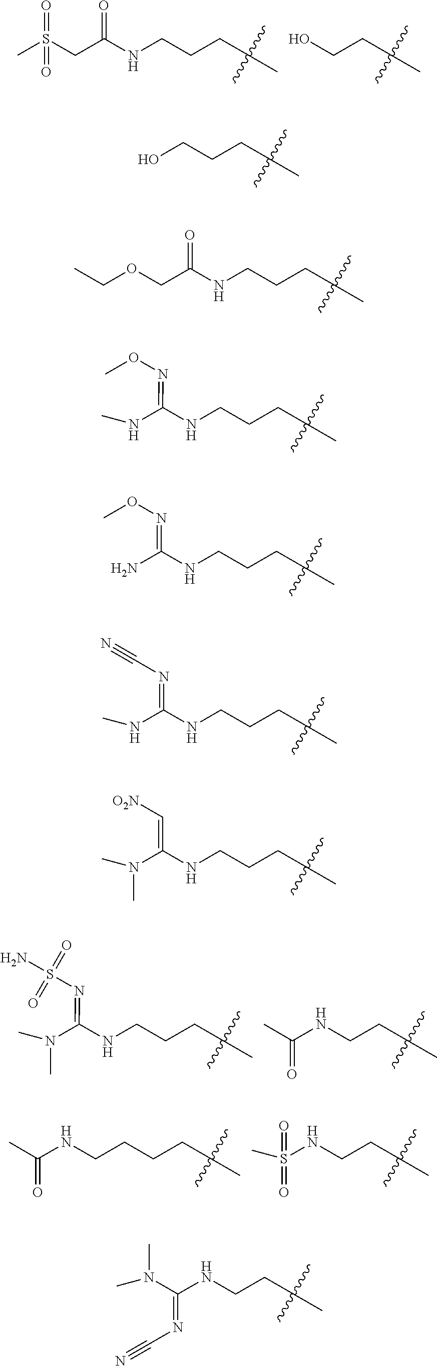

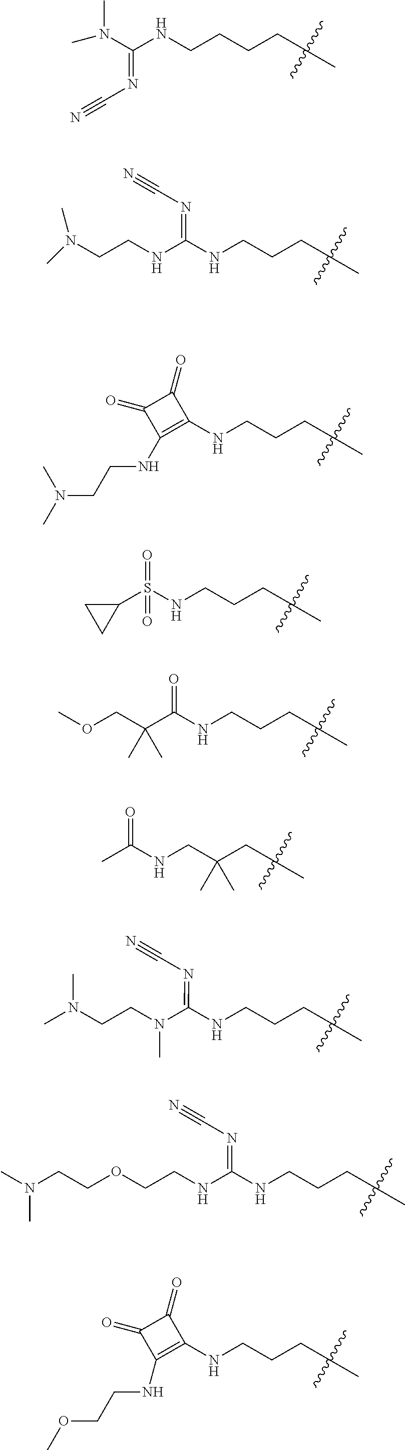

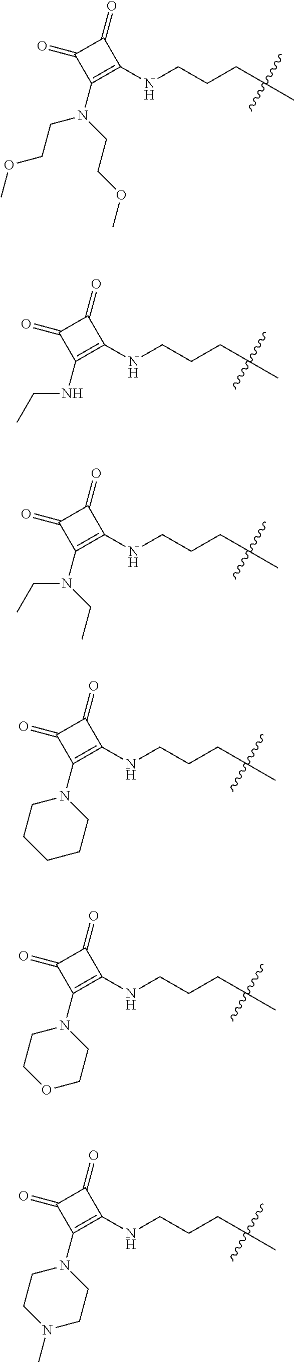 [SMILES: *CCCNc1c(N(CCOC)CCOC)c(=O)c1=O ; *CCCNc1c(NCC)c(=O)c1=O ; *CCCNc1c(N(CC)CC)c(=O)c1=O ; *CCCNc1c(N2CCCCC2)c(=O)c1=O ; *CCCNc1c(N2CCOCC2)c(=O)c1=O ; *CCCNc1c(N2CCN(C)CC2)c(=O)c1=O]

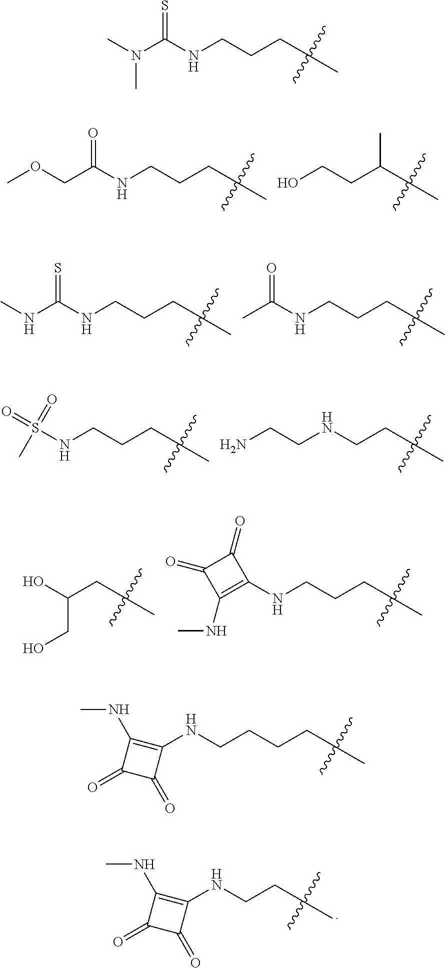

In some embodiments,

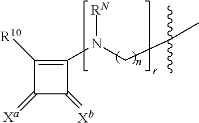

is selected from any of the following groups:

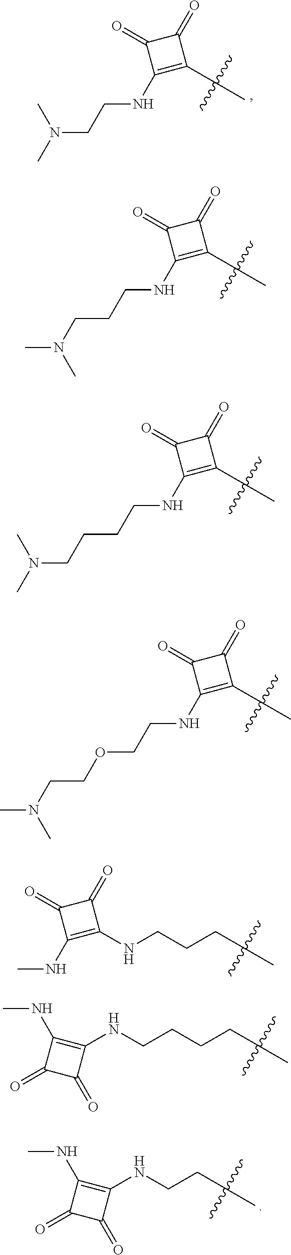

In some embodiments, a compound of Formula (III) further comprises an anion. As described herein, and anion can be any anion capable of reacting with an amine to form an ammonium salt. Examples include, but are not limited to, chloride, bromide, iodide, fluoride, acetate, formate, trifluoroacetate, difluoroacetate. trichloroacetate, and phosphate.

In some embodiments the compound of any of the formulae described herein is suitable for making a nanoparticle composition for intramuscular administration.

In some embodiments, R² and R³, together with the atom to which they are attached, form a heterocycle or carbocycle. In some embodiments, R² and R³, together with the atom to which they are attached, form a 5- to 14-membered aromatic or non-aromatic heterocycle having one or more heteroatoms selected from N, O, S, and P. In some embodiments, R² and R³, together with the atom to which they are attached, form an optionally substituted C₃₋₂₀ carbocycle (e.g., C₃₋₁₈ carbocycle, C₃₋₁₅ carbocycle, C₃₋₁₂ carbocycle, or C₃₋₁₀ carbocycle), either aromatic or non-aromatic. In some embodiments, R² and R³, together with the atom to which they are attached, form a C₃₋₆ carbocycle. In other embodiments, R² and R³, together with the atom to which they are attached, form a C₆ carbocycle, such as a cyclohexyl or phenyl group. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a cyclohexyl or phenyl group bearing one or more C₅ alkyl substitutions. In certain embodiments, the heterocycle or C₃₋₆ carbocycle formed by R² and R³, is substituted with a carbocycle groups. For example, R² and R³, together with the atom to which they are attached, may form a cyclohexyl or phenyl group that is substituted with cyclohexyl. In some embodiments, R² and R³, together with the atom to which they are attached, form a C₇₋₁₅ carbocycle, such as a cycloheptyl, cyclopentadecanyl, or naphthyl group.

In some embodiments, R⁴ is selected from —(CH₂)ₙQ and —(CH₂)ₙCHQR. In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(R)S(O)₂R⁸, —N(H)C(O)N(H)(R), —N(R)C(S)N(R)₂, —N(H)C(S)N(R)₂, —N(H)C(S)N(H)(R), and a heterocycle. In other embodiments, Q is selected from the group consisting of an imidazole, a pyrimidine, and a purine.

In some embodiments, R² and R³, together with the atom to which they are attached, form a heterocycle or carbocycle. In some embodiments, R² and R³, together with the atom to which they are attached, form a C₃₋₆ carbocycle. In some embodiments, R² and R³, together with the atom to which they are attached, form a C₆ carbocycle. In some embodiments, R² and R³, together with the atom to which they are attached, form a phenyl group. In some embodiments, R² and R³, together with the atom to which they are attached, form a cyclohexyl group. In some embodiments, R² and R³, together with the atom to which they are attached, form a heterocycle. In certain embodiments, the heterocycle or C₃₋₆ carbocycle is substituted with one or more alkyl groups (e.g., at the same ring atom or at adjacent or non-adjacent ring atoms). For example, R² and R³, together with the atom to which they are attached, may form a phenyl group bearing one or more C₅ alkyl substitutions.

In some embodiments, at least one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at most one occurrence of R⁵ and R⁶ is C₁₋₃ alkyl, e.g., methyl. In some embodiments, one of the R⁵ and R⁶ adjacent to M is C₁₋₃ alkyl, e.g., methyl, and the other is H. In some embodiments, one of the R⁵ and R⁶ adjacent to M is C₁₋₃ alkyl, e.g., methyl and the other is H, and M is —OC(O)— or —C(O)O—.

In some embodiments, at least one occurrence of R⁵ and R⁶ is methyl.

The compounds of any one of formula (VI), (VI-a), (VII), (VIIa), (VIIb), (VIIc), (VIId), (VIII), (VIIIa), (VIIIb), (VIIIc) or (VIIId) include one or more of the following features when applicable.

In some embodiments, r is 0. In some embodiments, r is 1.

In some embodiments, n is 2, 3, or 4. In some embodiments, n is 2. In some embodiments, n is 4. In some embodiments, n is not 3.

In some embodiments, R^{N} is H. In some embodiments, R^{N} is C₁₋₃ alkyl. For example, in some embodiments, R^{N} is C₁ alkyl. For example, in some embodiments, R^{N} is C₂ alkyl. For example, in some embodiments, R^{N} is C₂ alkyl.

In some embodiments, X^{a} is O. In some embodiments, X^{a} is S. In some embodiments, X^{b} is O. In some embodiments, X^{b} is S.

In some embodiments, R¹⁰ is selected from the group consisting of N(R)₂, —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments, R¹⁰ is selected from the group consisting of —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, and a heterocycle.

In some embodiments wherein R¹⁰ is —NH(CH₂)ₒN(R)₂, o is 2, 3, or 4.

In some embodiments wherein —NH(CH₂)ₚ₁O(CH₂)_{q1}N(R)₂, p¹ is 2. In some embodiments wherein —NH(CH₂)ₚ₁O(CH₂)_{q1}N(R)₂, q¹ is 2.

In some embodiments wherein R¹⁰ is —N((CH₂)ₛ₁OR)₂, s¹ is 2.

In some embodiments wherein R¹⁰ is —NH(CH₂)ₒN(R)₂, —NH(CH₂)ₚO(CH₂)_{q}N(R)₂, —NH(CH₂)ₛOR, or —N((CH₂)ₛOR)₂, R is H or C₁-C₃ alkyl. For example, in some embodiments, R is C₁ alkyl. For example, in some embodiments, R is C₂ alkyl. For example, in some embodiments, R is H. For example, in some embodiments, R is H and one R is C₁-C₃ alkyl. For example, in some embodiments, R is H and one R is C₁ alkyl. For example, in some embodiments, R is H and one R is C₂ alkyl. In some embodiments wherein R¹⁰ is —NH(CH₂)ₜ₁N(R)₂, —NH(CH₂)ₚ₁O(CH₂)_{q1}N(R)₂, —NH(CH₂)ₛ₁OR, or —N((CH₂)ₛ₁OR)₂, each R is C₂-C₄ alkyl.

For example, in some embodiments, one R is H and one R is C₂-C₄ alkyl. In some embodiments, R¹⁰ is a heterocycle. For example, in some embodiments, R¹⁰ is morpholinyl. For example, in some embodiments, R¹⁰ is methyhlpiperazinyl.

In some embodiments, each occurrence of R⁵ and R⁶ is H.

In some embodiments, the compound of Formula (I) is selected from the group consisting of:

| Cpd | Structure |
|---|---|
| I 1 | 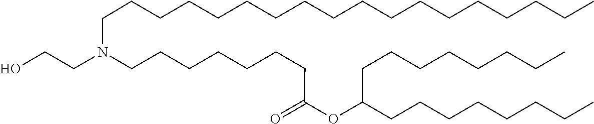 [SMILES: CCCCCCCCCCCCCCCCCCN(CCO)CCCCCCCC(=O)OC(CCCCCCCC)CCCCCCCC] |
| I 2 | 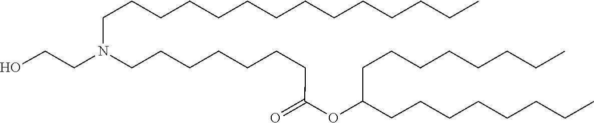 [SMILES: CCCCCCCCCCCCCCN(CCO)CCCCCCCC(=O)OC(CCCCCCCC)CCCCCCCC] |
| I 3 | 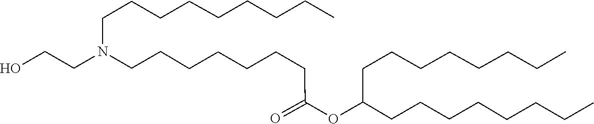 [SMILES: CCCCCCCCCN(CCO)CCCCCCCC(=O)OC(CCCCCCCC)CCCCCCCC] |
| I 4 | 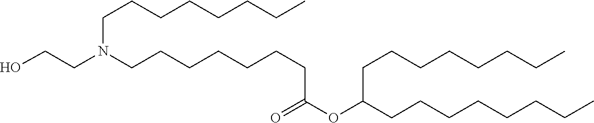 [SMILES: CCCCCCCCC(CCCCCCCC)OC(=O)CCCCCCCN(CCO)CCCCCCCC] |
| I 5 | 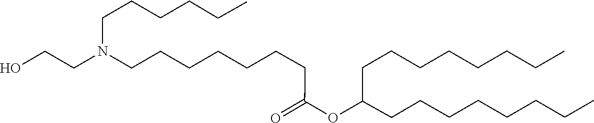 [SMILES: CCCCCCCCC(CCCCCCCC)OC(=O)CCCCCCCN(CCO)CCCCCC] |
| I 6 | 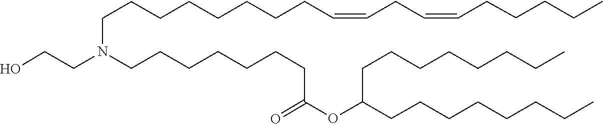 [SMILES: CCCCC/C=C\C/C=C\CCCCCCCCN(CCO)CCCCCCCC(=O)OC(CCCCCCCC)CCCCCCCC] |
| I 7 | 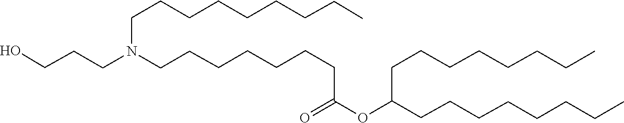 [SMILES: CCCCCCCCCN(CCCO)CCCCCCCC(=O)OC(CCCCCCCC)CCCCCCCC] |
| I 8 | 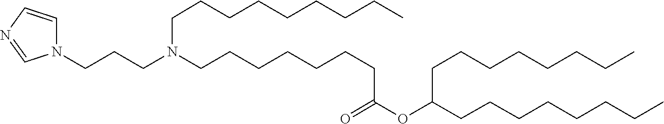 [SMILES: CCCCCCCCCN(CCCCCCCC(=O)OC(CCCCCCCC)CCCCCCCC)CCCn1ccnc1] |
| I 9 | 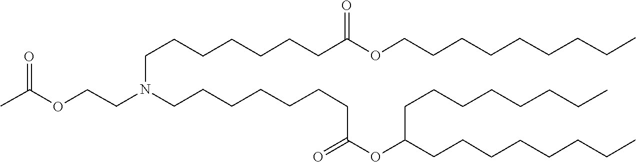 [SMILES: CCCCCCCCCOC(=O)CCCCCCCN(CCCCCCCC(=O)OC(CCCCCCCC)CCCCCCCC)CCOC(C)=O] |
| I 10 | 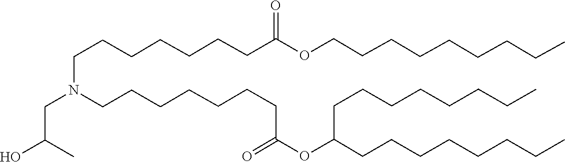 [SMILES: CCCCCCCCCOC(=O)CCCCCCCN(CCCCCCCC(=O)OC(CCCCCCCC)CCCCCCCC)CC(C)O] |
| I 11 | 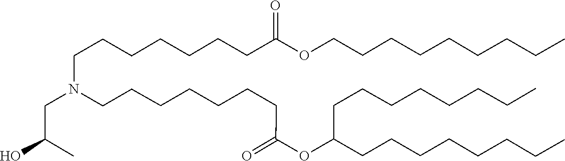 [SMILES: CCCCCCCCCOC(=O)CCCCCCCN(CCCCCCCC(=O)OC(CCCCCCCC)CCCCCCCC)C[C@@H](C)O] |
| I 12 | 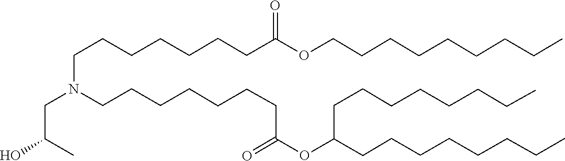 [SMILES: CCCCCCCCCOC(=O)CCCCCCCN(CCCCCCCC(=O)OC(CCCCCCCC)CCCCCCCC)C[C@H](C)O] |
| I 13 | 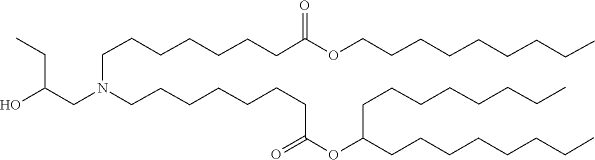 [SMILES: CCCCCCCCCOC(=O)CCCCCCCN(CCCCCCCC(=O)OC(CCCCCCCC)CCCCCCCC)CC(O)CC] |
| I 14 | 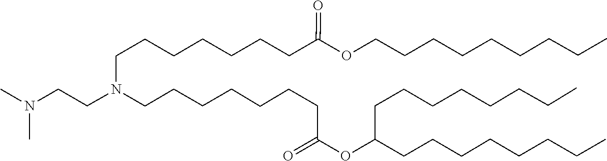 [SMILES: CCCCCCCCCOC(=O)CCCCCCCN(CCCCCCCC(=O)OC(CCCCCCCC)CCCCCCCC)CCN(C)C] |
| I 15 | 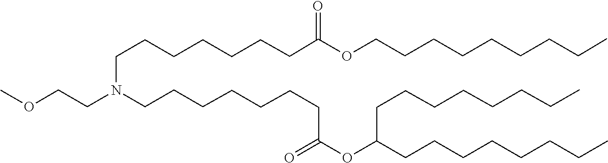 [SMILES: CCCCCCCCCOC(=O)CCCCCCCN(CCCCCCCC(=O)OC(CCCCCCCC)CCCCCCCC)CCOC] |
| I 16 | 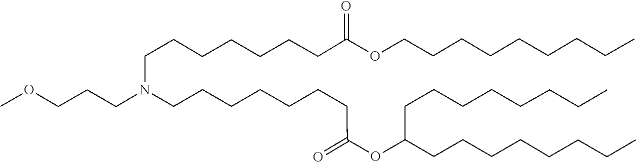 [SMILES: CCCCCCCCCOC(=O)CCCCCCCN(CCCCCCCC(=O)OC(CCCCCCCC)CCCCCCCC)CCCOC] |
| I 17 | 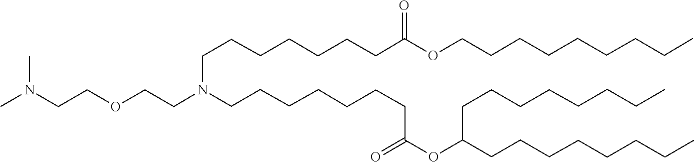 [SMILES: CCCCCCCCCOC(=O)CCCCCCCN(CCCCCCCC(=O)OC(CCCCCCCC)CCCCCCCC)CCOCCN(C)C] |
| I 18 | 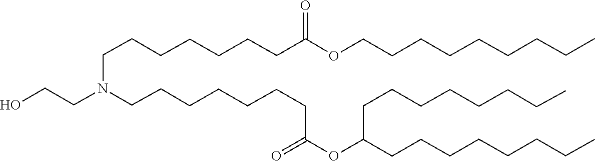 [SMILES: CCCCCCCCCOC(=O)CCCCCCCN(CCO)CCCCCCCC(=O)OC(CCCCCCCC)CCCCCCCC] |
| I 19 | 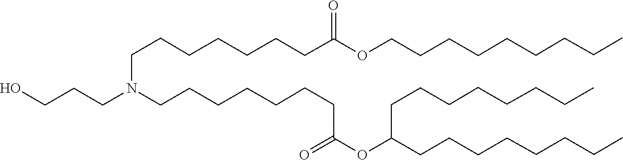 [SMILES: CCCCCCCCCOC(=O)CCCCCCCN(CCCO)CCCCCCCC(=O)OC(CCCCCCCC)CCCCCCCC] |
| I 20 | 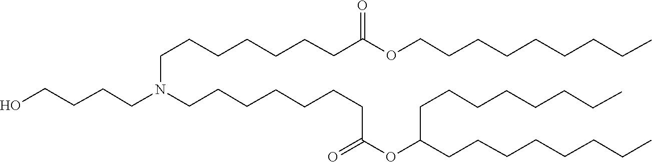 [SMILES: CCCCCCCCCOC(=O)CCCCCCCN(CCCCO)CCCCCCCC(=O)OC(CCCCCCCC)CCCCCCCC] |
| I 21 | 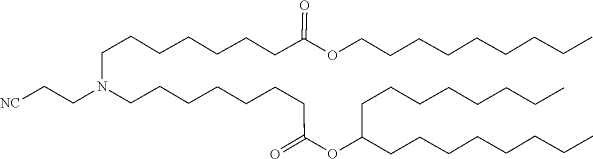 [SMILES: CCCCCCCCCOC(=O)CCCCCCCN(CCC#N)CCCCCCCC(=O)OC(CCCCCCCC)CCCCCCCC] |
| I 22 | 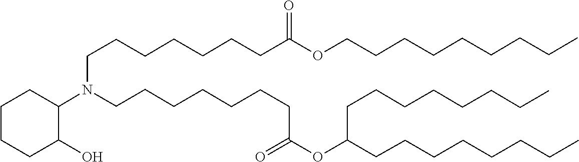 [SMILES: CCCCCCCCCOC(=O)CCCCCCCN(CCCCCCCC(=O)OC(CCCCCCCC)CCCCCCCC)C1CCCCC1O] |
| I 23 | 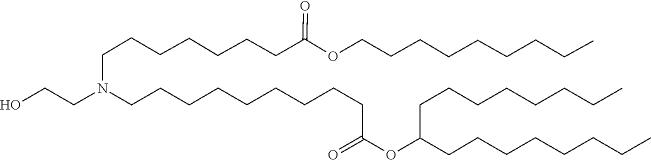 [SMILES: CCCCCCCCCOC(=O)CCCCCCCN(CCO)CCCCCCCCCC(=O)OC(CCCCCCCC)CCCCCCCC] |
| I 24 | 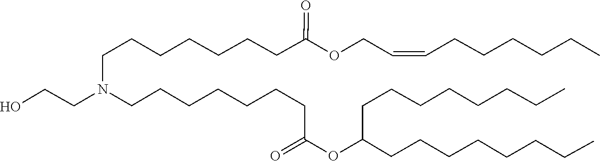 [SMILES: CCCCCC/C=C\COC(=O)CCCCCCCN(CCO)CCCCCCCC(=O)OC(CCCCCCCC)CCCCCCCC] |
| I 25 | 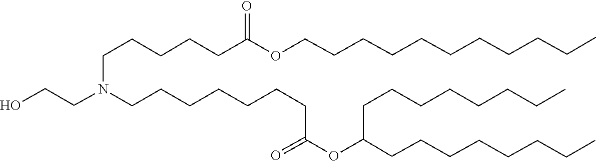 [SMILES: CCCCCCCCCCCOC(=O)CCCCCN(CCO)CCCCCCCC(=O)OC(CCCCCCCC)CCCCCCCC] |
| I 26 | 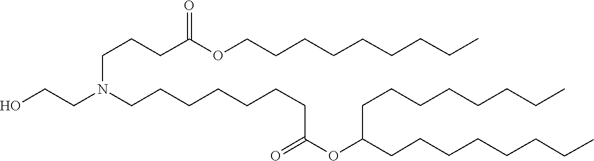 [SMILES: CCCCCCCCCOC(=O)CCCN(CCO)CCCCCCCC(=O)OC(CCCCCCCC)CCCCCCCC] |
| I 27 | 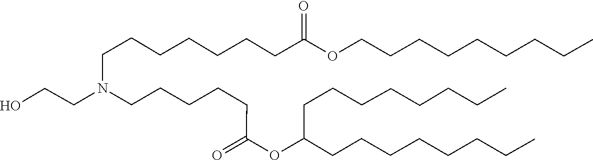 [SMILES: CCCCCCCCCOC(=O)CCCCCCCN(CCO)CCCCCC(=O)OC(CCCCCCCC)CCCCCCCC] |
| I 28 | 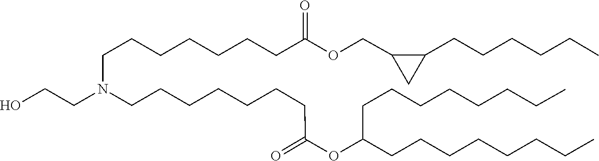 [SMILES: CCCCCCCCC(CCCCCCCC)OC(=O)CCCCCCCN(CCO)CCCCCCCC(=O)OCC1CC1CCCCCC] |
| I 29 | 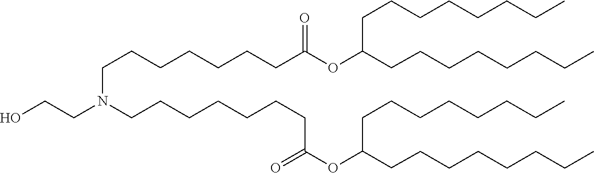 [SMILES: CCCCCCCCC(CCCCCCCC)OC(=O)CCCCCCCN(CCO)CCCCCCCC(=O)OC(CCCCCCCC)CCCCCCCC] |
| I 30 | 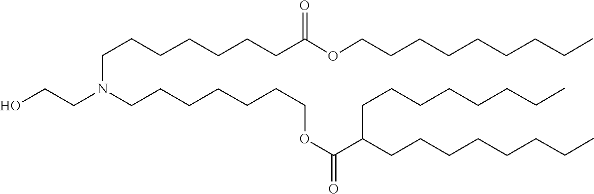 [SMILES: CCCCCCCCCOC(=O)CCCCCCCN(CCO)CCCCCCCOC(=O)C(CCCCCCCC)CCCCCCCC] |
| I 31 | 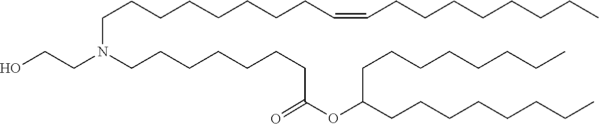 [SMILES: CCCCCCCC/C=C\CCCCCCCCN(CCO)CCCCCCCC(=O)OC(CCCCCCCC)CCCCCCCC] |
| I 32 | 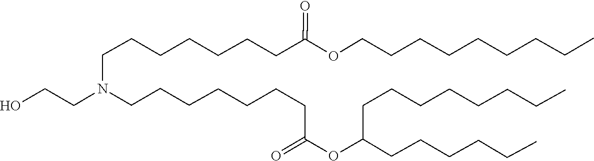 [SMILES: CCCCCCCCCOC(=O)CCCCCCCN(CCO)CCCCCCCC(=O)OC(CCCCCC)CCCCCCCC] |
| I 33 | 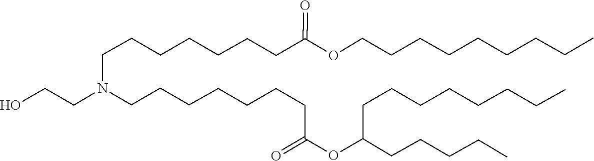 [SMILES: CCCCCCCCCOC(=O)CCCCCCCN(CCO)CCCCCCCC(=O)OC(CCCCC)CCCCCCCC] |
| I 34 | 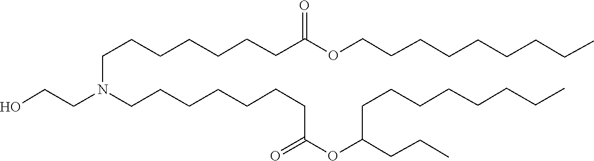 [SMILES: CCCCCCCCCOC(=O)CCCCCCCN(CCO)CCCCCCCC(=O)OC(CCC)CCCCCCCC] |
| I 35 | 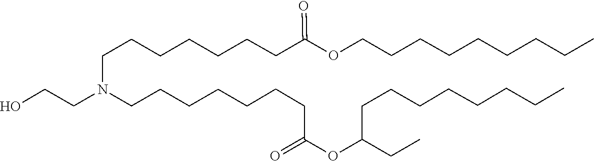 [SMILES: CCCCCCCCCOC(=O)CCCCCCCN(CCO)CCCCCCCC(=O)OC(CC)CCCCCCCC] |
| I 36 | 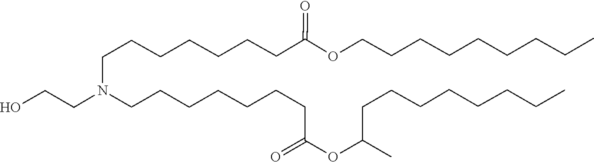 [SMILES: CCCCCCCCCOC(=O)CCCCCCCN(CCO)CCCCCCCC(=O)OC(C)CCCCCCCC] |
| I 37 | 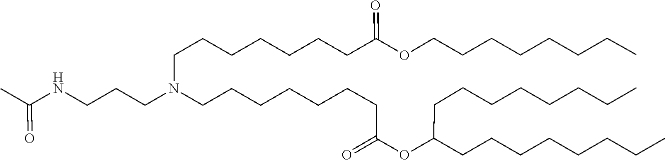 [SMILES: CCCCCCCCOC(=O)CCCCCCCN(CCCCCCCC(=O)OC(CCCCCCCC)CCCCCCCC)CCCNC(C)=O] |
| I 38 | 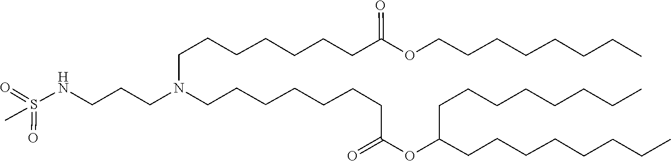 [SMILES: CCCCCCCCOC(=O)CCCCCCCN(CCCCCCCC(=O)OC(CCCCCCCC)CCCCCCCC)CCCNS(C)(=O)=O] |
| I 39 | 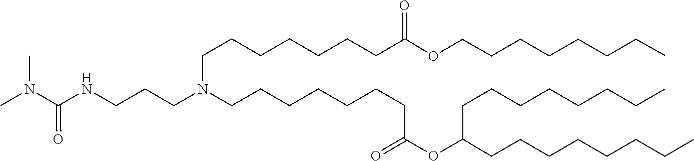 [SMILES: CCCCCCCCOC(=O)CCCCCCCN(CCCCCCCC(=O)OC(CCCCCCCC)CCCCCCCC)CCCNC(=O)N(C)C] |
| I 40 | 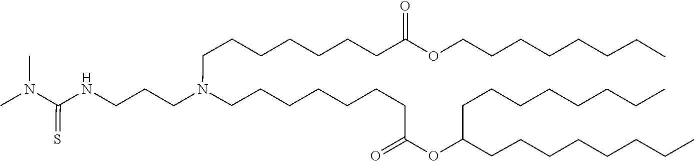 [SMILES: CCCCCCCCOC(=O)CCCCCCCN(CCCCCCCC(=O)OC(CCCCCCCC)CCCCCCCC)CCCNC(=S)N(C)C] |
| I 41 | 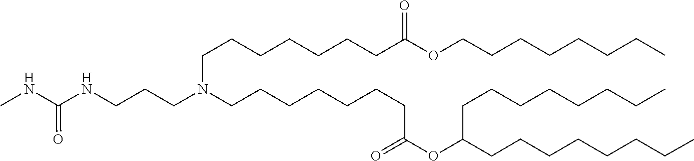 [SMILES: CCCCCCCCOC(=O)CCCCCCCN(CCCCCCCC(=O)OC(CCCCCCCC)CCCCCCCC)CCCNC(=O)NC] |
| I 42 | 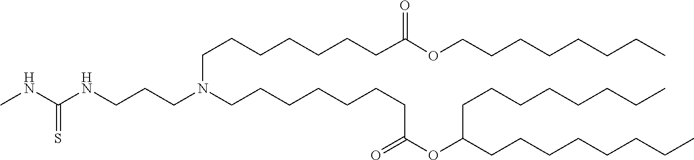 [SMILES: CCCCCCCCOC(=O)CCCCCCCN(CCCCCCCC(=O)OC(CCCCCCCC)CCCCCCCC)CCCNC(=S)NC] |
| I 43 | 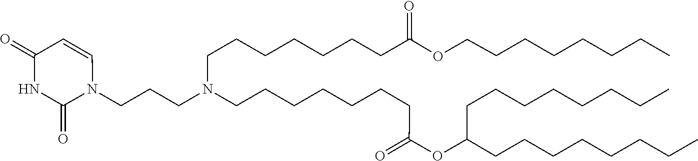 [SMILES: CCCCCCCCOC(=O)CCCCCCCN(CCCCCCCC(=O)OC(CCCCCCCC)CCCCCCCC)CCCn1ccc(=O)[nH]c1=O] |
| I 44 | 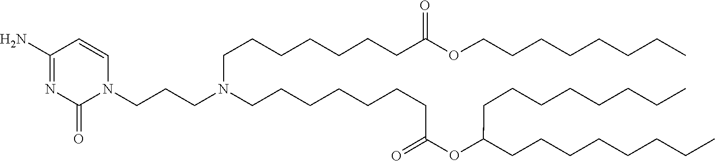 [SMILES: CCCCCCCCOC(=O)CCCCCCCN(CCCCCCCC(=O)OC(CCCCCCCC)CCCCCCCC)CCCn1ccc(N)nc1=O] |
| I 45 | 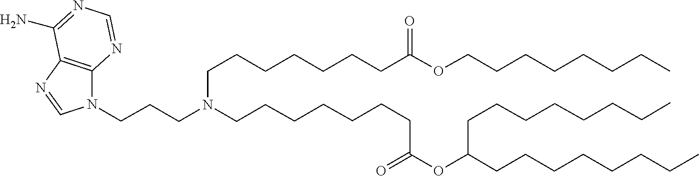 [SMILES: CCCCCCCCOC(=O)CCCCCCCN(CCCCCCCC(=O)OC(CCCCCCCC)CCCCCCCC)CCCn1cnc2c(N)ncnc21] |
| I 46 | 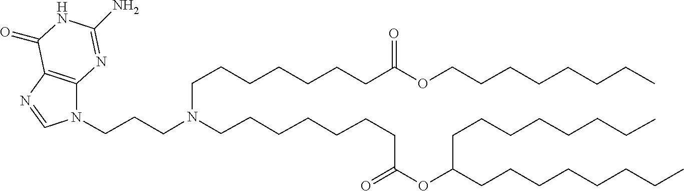 [SMILES: CCCCCCCCOC(=O)CCCCCCCN(CCCCCCCC(=O)OC(CCCCCCCC)CCCCCCCC)CCCn1cnc2c(=O)[nH]c(N)nc21] |
| I 47 | 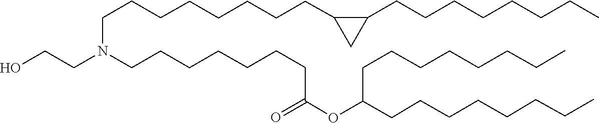 [SMILES: CCCCCCCCC(CCCCCCCC)OC(=O)CCCCCCCN(CCO)CCCCCCCCC1CC1CCCCCCCC] |
| I 48 | 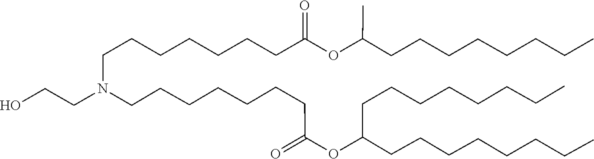 [SMILES: CCCCCCCCC(C)OC(=O)CCCCCCCN(CCO)CCCCCCCC(=O)OC(CCCCCCCC)CCCCCCCC] |
| I 49 | 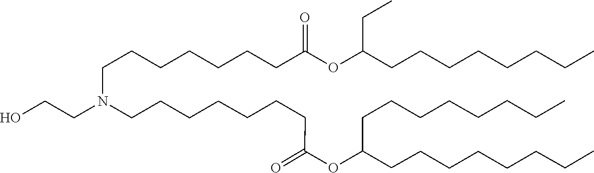 [SMILES: CCCCCCCCC(CC)OC(=O)CCCCCCCN(CCO)CCCCCCCC(=O)OC(CCCCCCCC)CCCCCCCC] |
| I 50 | 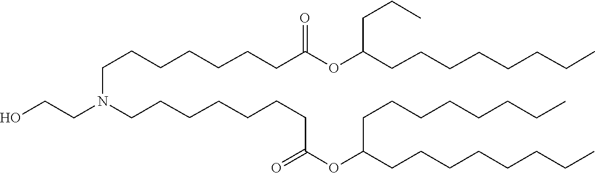 [SMILES: CCCCCCCCC(CCC)OC(=O)CCCCCCCN(CCO)CCCCCCCC(=O)OC(CCCCCCCC)CCCCCCCC] |
| I 51 | 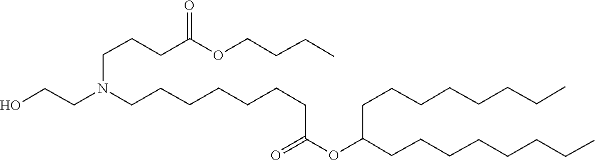 [SMILES: CCCCCCCCC(CCCCCCCC)OC(=O)CCCCCCCN(CCO)CCCC(=O)OCCCC] |
| I 52 | 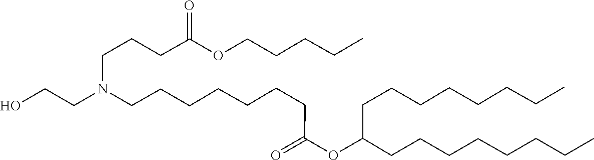 [SMILES: CCCCCCCCC(CCCCCCCC)OC(=O)CCCCCCCN(CCO)CCCC(=O)OCCCCC] |
| I 53 | 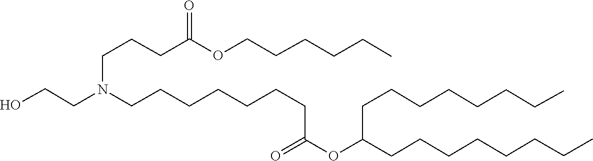 [SMILES: CCCCCCCCC(CCCCCCCC)OC(=O)CCCCCCCN(CCO)CCCC(=O)OCCCCCC] |
| I 54 | 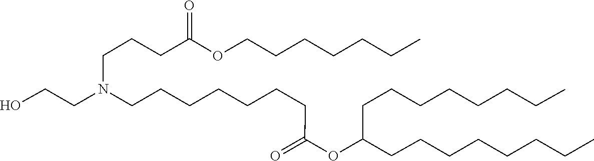 [SMILES: CCCCCCCCC(CCCCCCCC)OC(=O)CCCCCCCN(CCO)CCCC(=O)OCCCCCCC] |
| I 55 | 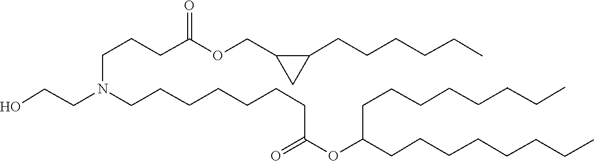 [SMILES: CCCCCCCCC(CCCCCCCC)OC(=O)CCCCCCCN(CCO)CCCC(=O)OCC1CC1CCCCCC] |
| I 56 | 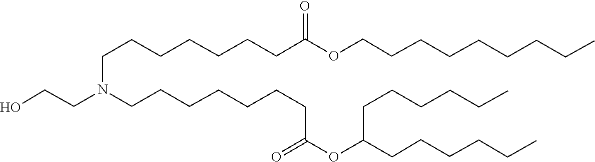 [SMILES: CCCCCCCCCOC(=O)CCCCCCCN(CCO)CCCCCCCC(=O)OC(CCCCCC)CCCCCC] |
| I 57 | 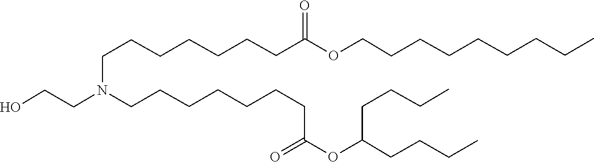 [SMILES: CCCCCCCCCOC(=O)CCCCCCCN(CCO)CCCCCCCC(=O)OC(CCCC)CCCC] |
| I 58 | 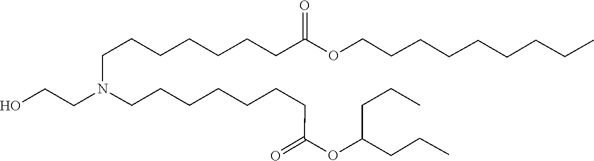 [SMILES: CCCCCCCCCOC(=O)CCCCCCCN(CCO)CCCCCCCC(=O)OC(CCC)CCC] |
| I 59 | 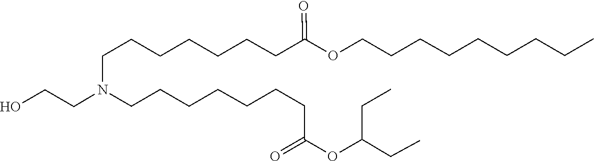 [SMILES: CCCCCCCCCOC(=O)CCCCCCCN(CCO)CCCCCCCC(=O)OC(CC)CC] |
| I 60 | 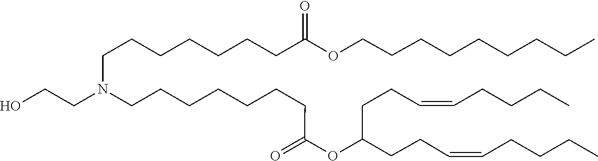 [SMILES: CCCC/C=C\CCC(CC/C=C\CCCC)OC(=O)CCCCCCCN(CCO)CCCCCCCC(=O)OCCCCCCCCC] |
| I 61 | 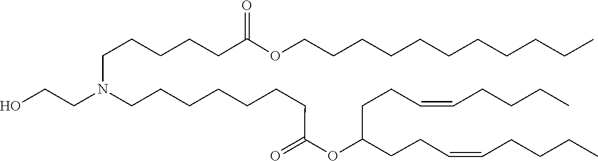 [SMILES: CCCC/C=C\CCC(CC/C=C\CCCC)OC(=O)CCCCCCCN(CCO)CCCCCC(=O)OCCCCCCCCCCC] |

In further embodiments, the compound of Formula (I I) is selected from the group consisting of:

| Cpd | Structure |
|---|---|
| I 62 | 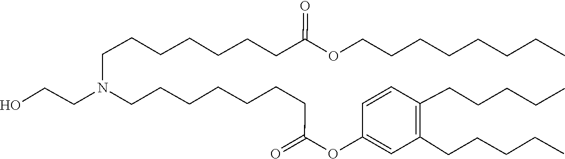 [SMILES: CCCCCCCCOC(=O)CCCCCCCN(CCO)CCCCCCCC(=O)Oc1ccc(CCCCC)c(CCCCC)c1] |
| I 63 | 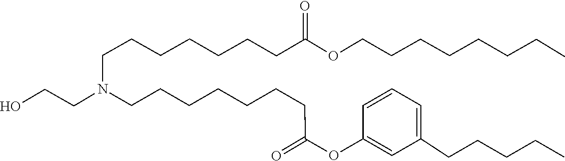 [SMILES: CCCCCCCCOC(=O)CCCCCCCN(CCO)CCCCCCCC(=O)Oc1cccc(CCCCC)c1] |
| I 64 | 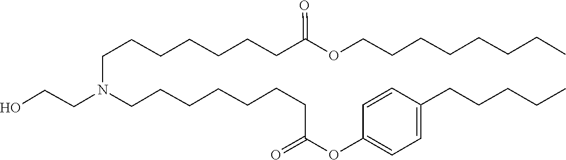 [SMILES: CCCCCCCCOC(=O)CCCCCCCN(CCO)CCCCCCCC(=O)Oc1ccc(CCCCC)cc1] |

In some embodiments, the compound of Formula (I I) or Formula (I IV) is selected from the group consisting of:

| Cpd | Structure |
|---|---|
| I 65 | 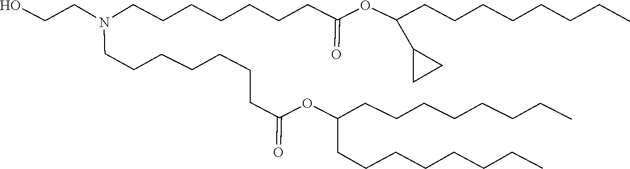 [SMILES: CCCCCCCCC(CCCCCCCC)OC(=O)CCCCCCCN(CCO)CCCCCCCC(=O)OC(CCCCCCCC)C1CC1] |
| I 66 | 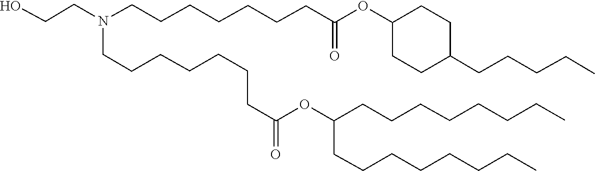 [SMILES: CCCCCCCCC(CCCCCCCC)OC(=O)CCCCCCCN(CCO)CCCCCCCC(=O)OC1CCC(CCCCC)CC1] |
| I 67 | 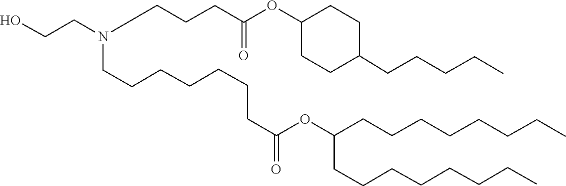 [SMILES: CCCCCCCCC(CCCCCCCC)OC(=O)CCCCCCCN(CCO)CCCC(=O)OC1CCC(CCCCC)CC1] |
| I 68 | 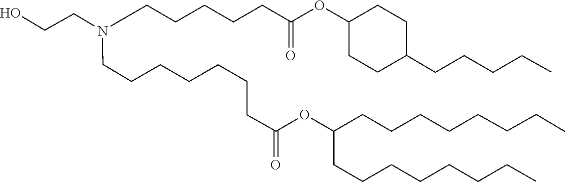 [SMILES: CCCCCCCCC(CCCCCCCC)OC(=O)CCCCCCCN(CCO)CCCCCC(=O)OC1CCC(CCCCC)CC1] |
| I 69 | 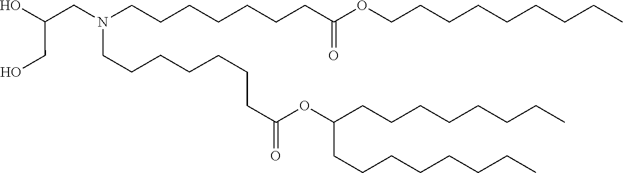 [SMILES: CCCCCCCCCOC(=O)CCCCCCCN(CCCCCCCC(=O)OC(CCCCCCCC)CCCCCCCC)CC(O)CO] |
| I 70 | 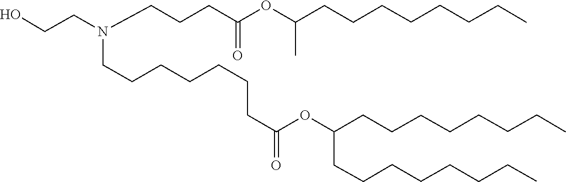 [SMILES: CCCCCCCCC(C)OC(=O)CCCN(CCO)CCCCCCCC(=O)OC(CCCCCCCC)CCCCCCCC] |
| I 71 | 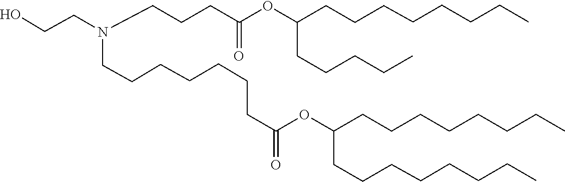 [SMILES: CCCCCCCCC(CCCCCCCC)OC(=O)CCCCCCCN(CCO)CCCC(=O)OC(CCCCC)CCCCCCCC] |
| I 72 | 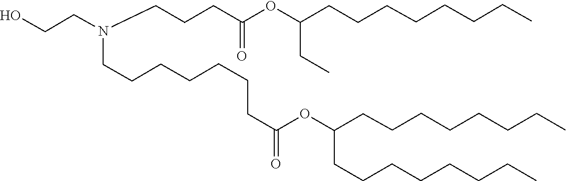 [SMILES: CCCCCCCCC(CC)OC(=O)CCCN(CCO)CCCCCCCC(=O)OC(CCCCCCCC)CCCCCCCC] |
| I 73 | 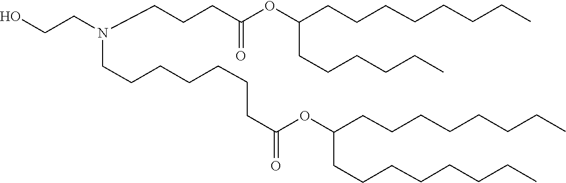 [SMILES: CCCCCCCCC(CCCCCCCC)OC(=O)CCCCCCCN(CCO)CCCC(=O)OC(CCCCCC)CCCCCCCC] |
| I 74 | 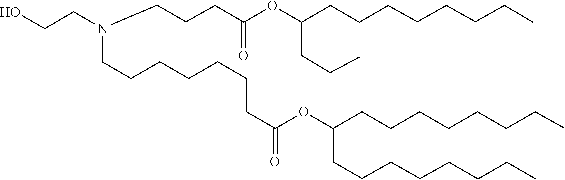 [SMILES: CCCCCCCCC(CCC)OC(=O)CCCN(CCO)CCCCCCCC(=O)OC(CCCCCCCC)CCCCCCCC] |
| I 75 | 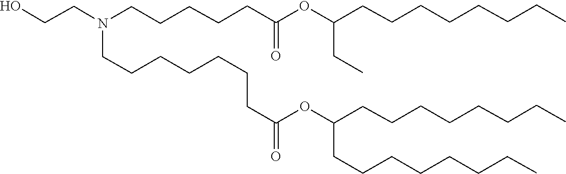 [SMILES: CCCCCCCCC(CC)OC(=O)CCCCCN(CCO)CCCCCCCC(=O)OC(CCCCCCCC)CCCCCCCC] |
| I 76 | 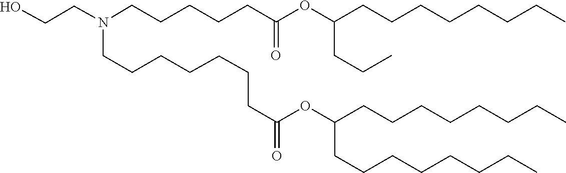 [SMILES: CCCCCCCCC(CCC)OC(=O)CCCCCN(CCO)CCCCCCCC(=O)OC(CCCCCCCC)CCCCCCCC] |
| I 77 | 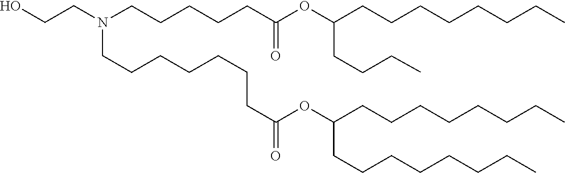 [SMILES: CCCCCCCCC(CCCC)OC(=O)CCCCCN(CCO)CCCCCCCC(=O)OC(CCCCCCCC)CCCCCCCC] |
| I 78 | 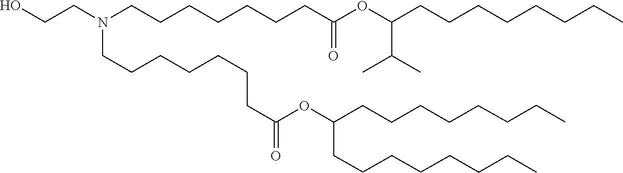 [SMILES: CCCCCCCCC(CCCCCCCC)OC(=O)CCCCCCCN(CCO)CCCCCCCC(=O)OC(CCCCCCCC)C(C)C] |
| I 79 | 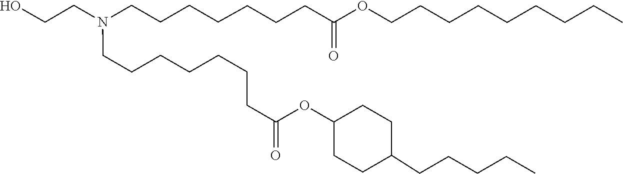 [SMILES: CCCCCCCCCOC(=O)CCCCCCCN(CCO)CCCCCCCC(=O)OC1CCC(CCCCC)CC1] |
| I 80 | 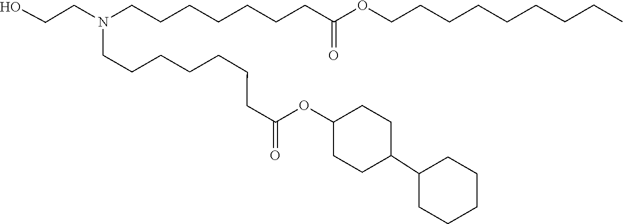 [SMILES: CCCCCCCCCOC(=O)CCCCCCCN(CCO)CCCCCCCC(=O)OC1CCC(C2CCCCC2)CC1] |
| I 81 | 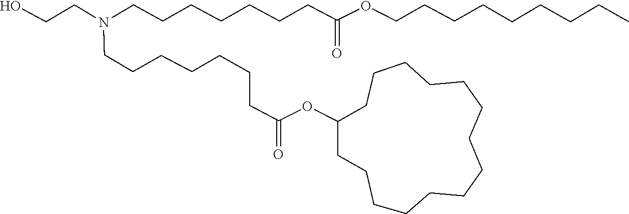 [SMILES: CCCCCCCCCOC(=O)CCCCCCCN(CCO)CCCCCCCC(=O)OC1CCCCCCCCCCCCCC1] |
| I 82 | 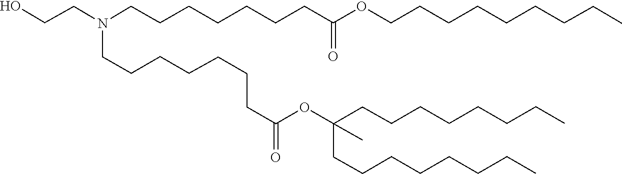 [SMILES: CCCCCCCCCOC(=O)CCCCCCCN(CCO)CCCCCCCC(=O)OC(C)(CCCCCCCC)CCCCCCCC] |
| I 83 | 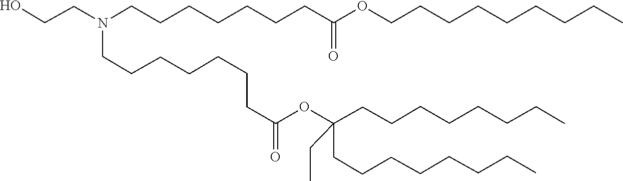 [SMILES: CCCCCCCCCOC(=O)CCCCCCCN(CCO)CCCCCCCC(=O)OC(CC)(CCCCCCCC)CCCCCCCC] |
| I 84 | 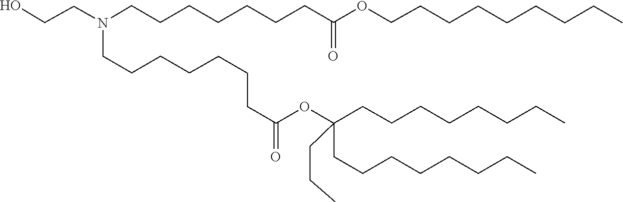 [SMILES: CCCCCCCCCOC(=O)CCCCCCCN(CCO)CCCCCCCC(=O)OC(CCC)(CCCCCCCC)CCCCCCCC] |
| I 85 | 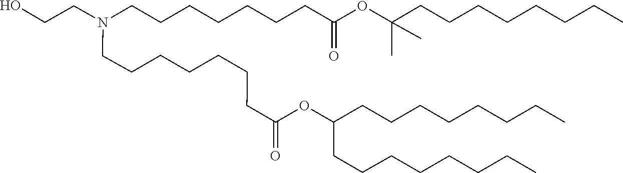 [SMILES: CCCCCCCCC(CCCCCCCC)OC(=O)CCCCCCCN(CCO)CCCCCCCC(=O)OC(C)(C)CCCCCCCC] |
| I 86 | 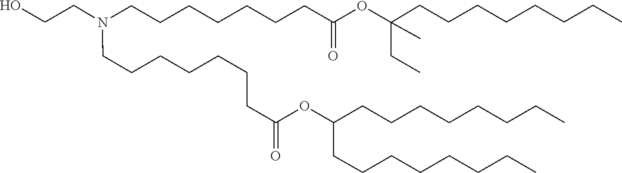 [SMILES: CCCCCCCCC(CCCCCCCC)OC(=O)CCCCCCCN(CCO)CCCCCCCC(=O)OC(C)(CC)CCCCCCCC] |
| I 87 | 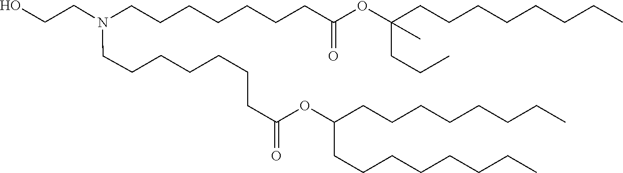 [SMILES: CCCCCCCCC(CCCCCCCC)OC(=O)CCCCCCCN(CCO)CCCCCCCC(=O)OC(C)(CCC)CCCCCCCC] |
| I 88 | 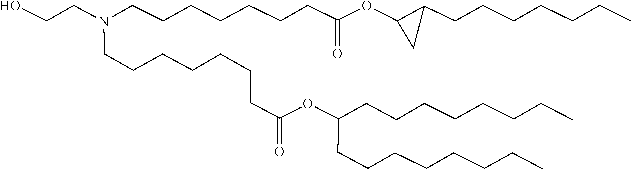 [SMILES: CCCCCCCCC(CCCCCCCC)OC(=O)CCCCCCCN(CCO)CCCCCCCC(=O)OC1CC1CCCCCCC] |
| I 89 | 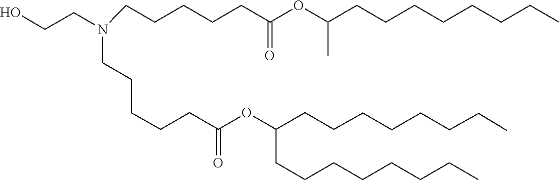 [SMILES: CCCCCCCCC(C)OC(=O)CCCCCN(CCO)CCCCCC(=O)OC(CCCCCCCC)CCCCCCCC] |
| I 90 | 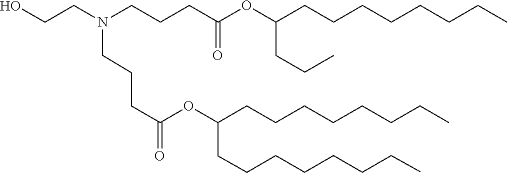 [SMILES: CCCCCCCCC(CCC)OC(=O)CCCN(CCO)CCCC(=O)OC(CCCCCCCC)CCCCCCCC] |
| I 91 | 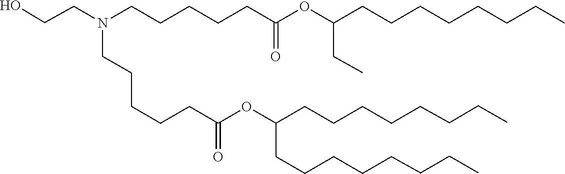 [SMILES: CCCCCCCCC(CC)OC(=O)CCCCCN(CCO)CCCCCC(=O)OC(CCCCCCCC)CCCCCCCC] |
| I 92 | 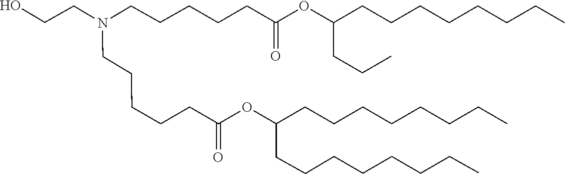 [SMILES: CCCCCCCCC(CCC)OC(=O)CCCCCN(CCO)CCCCCC(=O)OC(CCCCCCCC)CCCCCCCC] |
| I 93 | 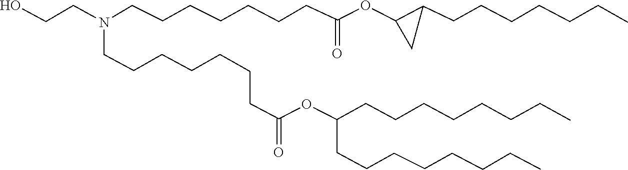 [SMILES: CCCCCCCCC(CCCCCCCC)OC(=O)CCCCCCCN(CCO)CCCCCCCC(=O)OC1CC1CCCCCCC] |
| I 94 | 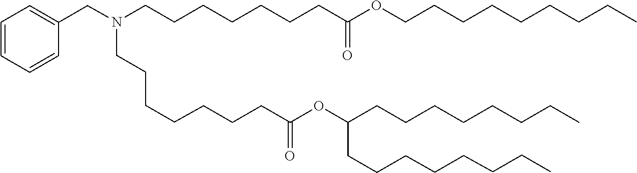 [SMILES: CCCCCCCCCOC(=O)CCCCCCCN(CCCCCCCC(=O)OC(CCCCCCCC)CCCCCCCC)Cc1ccccc1] |
| I 95 | 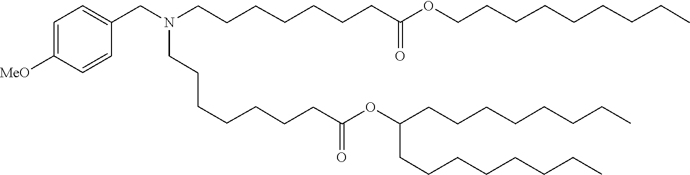 [SMILES: CCCCCCCCCOC(=O)CCCCCCCN(CCCCCCCC(=O)OC(CCCCCCCC)CCCCCCCC)Cc1ccc(OC)cc1] |
| I 96 | 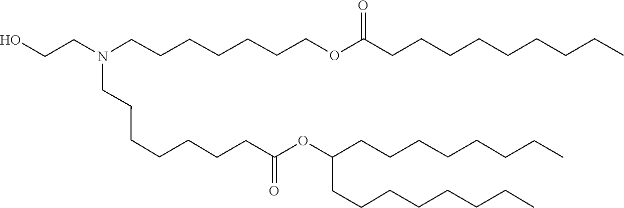 [SMILES: CCCCCCCCCC(=O)OCCCCCCCN(CCO)CCCCCCCC(=O)OC(CCCCCCCC)CCCCCCCC] |
| I 97 | 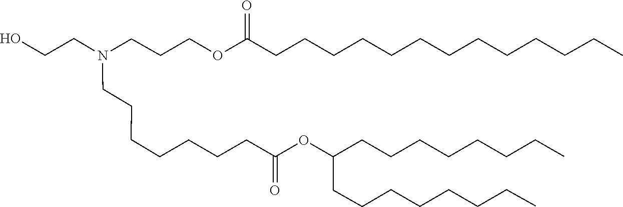 [SMILES: CCCCCCCCCCCCCC(=O)OCCCN(CCO)CCCCCCCC(=O)OC(CCCCCCCC)CCCCCCCC] |
| I 98 | 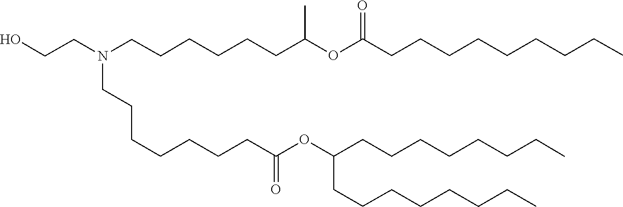 [SMILES: CCCCCCCCCC(=O)OC(C)CCCCCCN(CCO)CCCCCCCC(=O)OC(CCCCCCCC)CCCCCCCC] |
| I 99 | 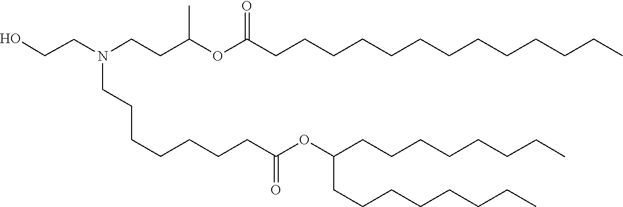 [SMILES: CCCCCCCCCCCCCC(=O)OC(C)CCN(CCO)CCCCCCCC(=O)OC(CCCCCCCC)CCCCCCCC] |
| I 100 | 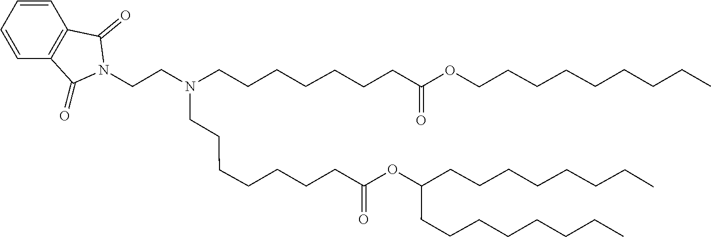 [SMILES: CCCCCCCCCOC(=O)CCCCCCCN(CCCCCCCC(=O)OC(CCCCCCCC)CCCCCCCC)CCN1C(=O)c2ccccc2C1=O] |
| I 10I 1 | 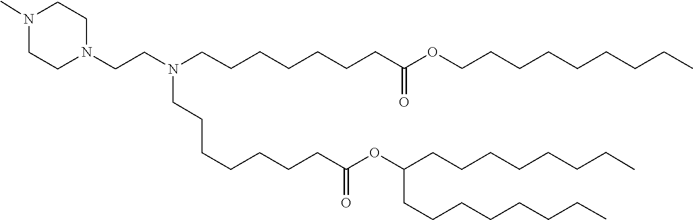 [SMILES: CCCCCCCCCOC(=O)CCCCCCCN(CCCCCCCC(=O)OC(CCCCCCCC)CCCCCCCC)CCN1CCN(C)CC1] |
| I 102 | 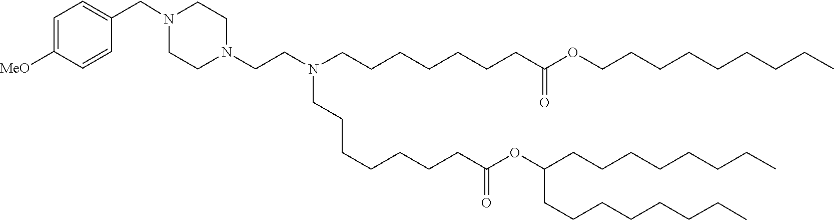 [SMILES: CCCCCCCCCOC(=O)CCCCCCCN(CCCCCCCC(=O)OC(CCCCCCCC)CCCCCCCC)CCN1CCN(Cc2ccc(OC)cc2)CC1] |
| I 103 | 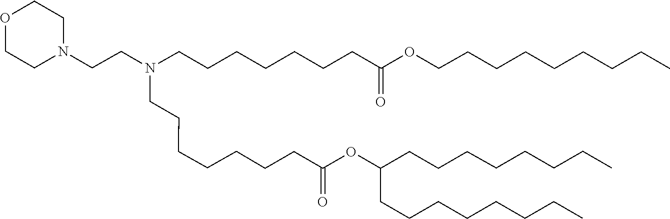 [SMILES: CCCCCCCCCOC(=O)CCCCCCCN(CCCCCCCC(=O)OC(CCCCCCCC)CCCCCCCC)CCN1CCOCC1] |
| I 104 | 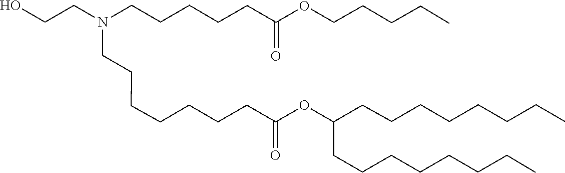 [SMILES: CCCCCCCCC(CCCCCCCC)OC(=O)CCCCCCCN(CCO)CCCCCC(=O)OCCCCC] |
| I 105 | 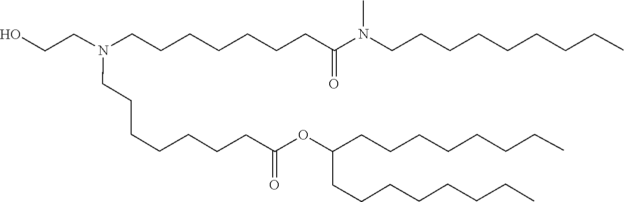 [SMILES: CCCCCCCCCN(C)C(=O)CCCCCCCN(CCO)CCCCCCCC(=O)OC(CCCCCCCC)CCCCCCCC] |
| I 106 | 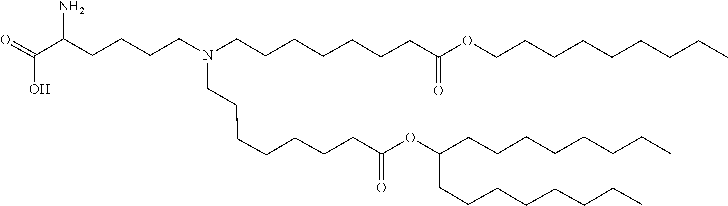 [SMILES: CCCCCCCCCOC(=O)CCCCCCCN(CCCCCCCC(=O)OC(CCCCCCCC)CCCCCCCC)CCCCC(N)C(=O)O] |
| I 107 | 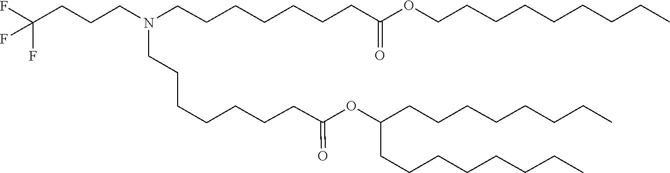 [SMILES: CCCCCCCCCOC(=O)CCCCCCCN(CCCCCCCC(=O)OC(CCCCCCCC)CCCCCCCC)CCCC(F)(F)F] |
| I 108 | 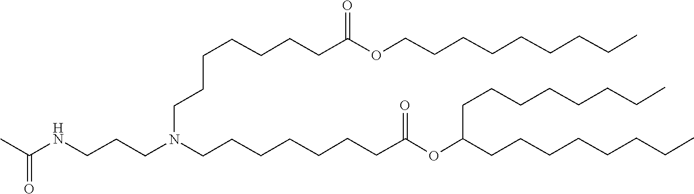 [SMILES: CCCCCCCCCOC(=O)CCCCCCCN(CCCCCCCC(=O)OC(CCCCCCCC)CCCCCCCC)CCCNC(C)=O] |
| I 109 | 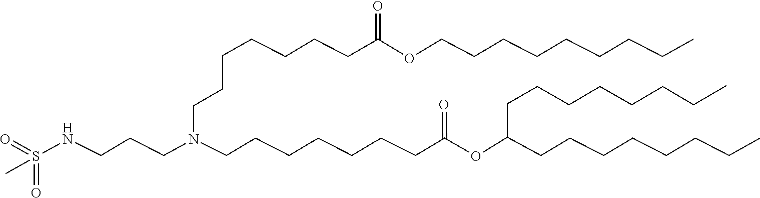 [SMILES: CCCCCCCCCOC(=O)CCCCCCCN(CCCCCCCC(=O)OC(CCCCCCCC)CCCCCCCC)CCCNS(C)(=O)=O] |
| I 110 | 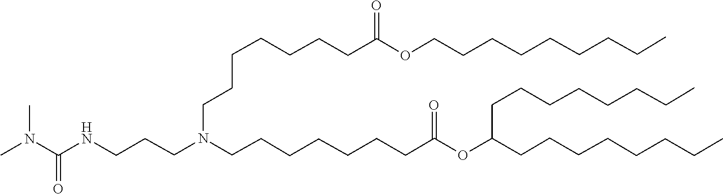 [SMILES: CCCCCCCCCOC(=O)CCCCCCCN(CCCCCCCC(=O)OC(CCCCCCCC)CCCCCCCC)CCCNC(=O)N(C)C] |
| I 111 | 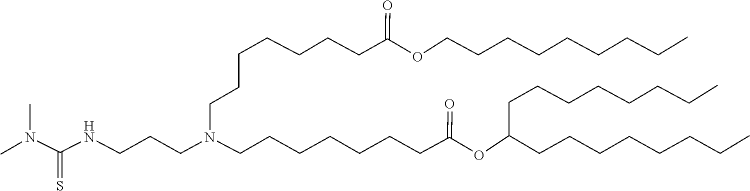 [SMILES: CCCCCCCCCOC(=O)CCCCCCCN(CCCCCCCC(=O)OC(CCCCCCCC)CCCCCCCC)CCCNC(=S)N(C)C] |
| I 112 | 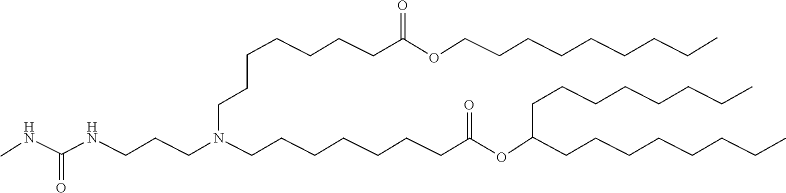 [SMILES: CCCCCCCCCOC(=O)CCCCCCCN(CCCCCCCC(=O)OC(CCCCCCCC)CCCCCCCC)CCCNC(=O)NC] |
| I 113 | 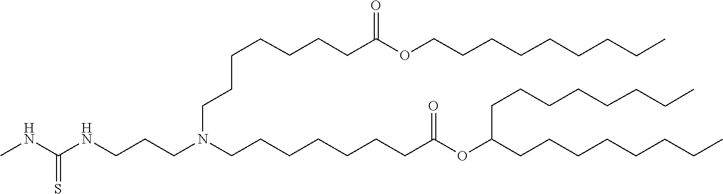 [SMILES: CCCCCCCCCOC(=O)CCCCCCCN(CCCCCCCC(=O)OC(CCCCCCCC)CCCCCCCC)CCCNC(=S)NC] |
| I 114 | 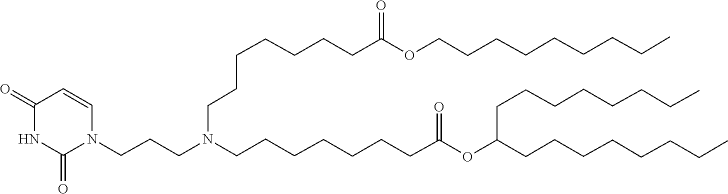 [SMILES: CCCCCCCCCOC(=O)CCCCCCCN(CCCCCCCC(=O)OC(CCCCCCCC)CCCCCCCC)CCCn1ccc(=O)[nH]c1=O] |
| I 115 | 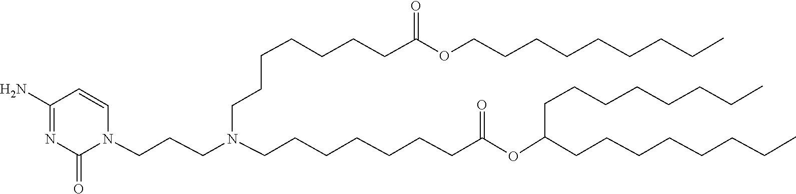 [SMILES: CCCCCCCCCOC(=O)CCCCCCCN(CCCCCCCC(=O)OC(CCCCCCCC)CCCCCCCC)CCCn1ccc(N)nc1=O] |
| I 116 | 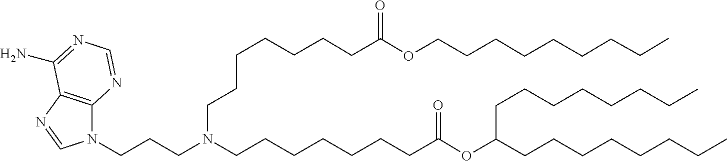 [SMILES: CCCCCCCCCOC(=O)CCCCCCCN(CCCCCCCC(=O)OC(CCCCCCCC)CCCCCCCC)CCCn1cnc2c(N)ncnc21] |
| I 117 | 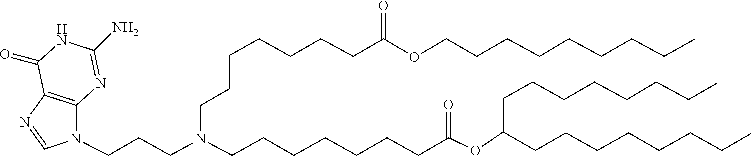 [SMILES: CCCCCCCCCOC(=O)CCCCCCCN(CCCCCCCC(=O)OC(CCCCCCCC)CCCCCCCC)CCCn1cnc2c(=O)[nH]c(N)nc21] |
| I 118 | 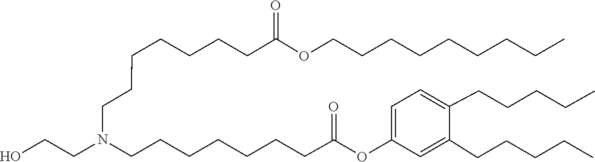 [SMILES: CCCCCCCCCOC(=O)CCCCCCCN(CCO)CCCCCCCC(=O)Oc1ccc(CCCCC)c(CCCCC)c1] |
| I 119 | 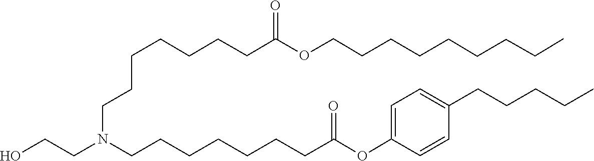 [SMILES: CCCCCCCCCOC(=O)CCCCCCCN(CCO)CCCCCCCC(=O)Oc1ccc(CCCCC)cc1] |
| I 120 | 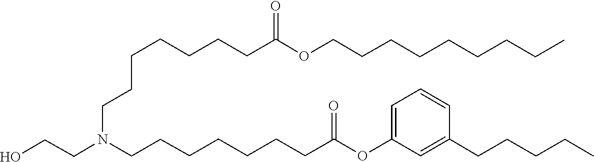 [SMILES: CCCCCCCCCOC(=O)CCCCCCCN(CCO)CCCCCCCC(=O)Oc1cccc(CCCCC)c1] |
| I 121 | 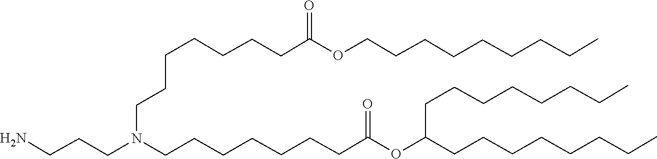 [SMILES: CCCCCCCCCOC(=O)CCCCCCCN(CCCN)CCCCCCCC(=O)OC(CCCCCCCC)CCCCCCCC] |
| I 122 | 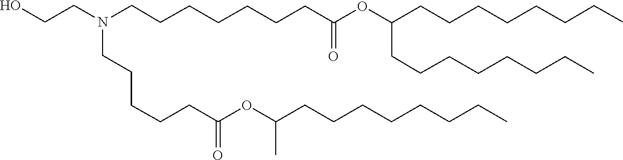 [SMILES: CCCCCCCCC(C)OC(=O)CCCCCN(CCO)CCCCCCCC(=O)OC(CCCCCCCC)CCCCCCCC] |
| I 123 | 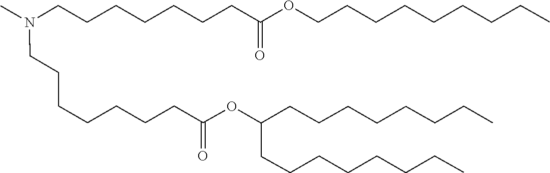 [SMILES: CCCCCCCCCOC(=O)CCCCCCCN(C)CCCCCCCC(=O)OC(CCCCCCCC)CCCCCCCC] |
| I 124 | 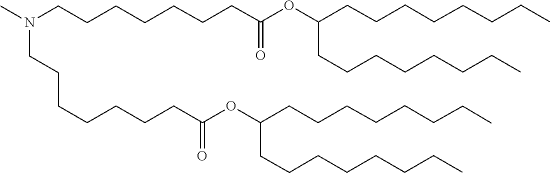 [SMILES: CCCCCCCCC(CCCCCCCC)OC(=O)CCCCCCCN(C)CCCCCCCC(=O)OC(CCCCCCCC)CCCCCCCC] |
| I 125 | 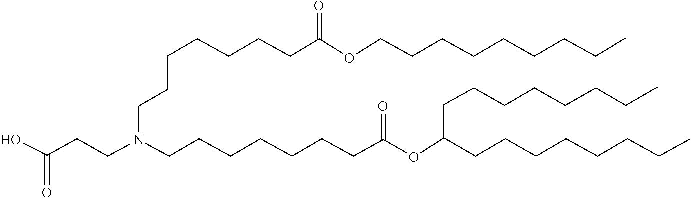 [SMILES: CCCCCCCCCOC(=O)CCCCCCCN(CCCCCCCC(=O)OC(CCCCCCCC)CCCCCCCC)CCC(=O)O] |
| I 126 | 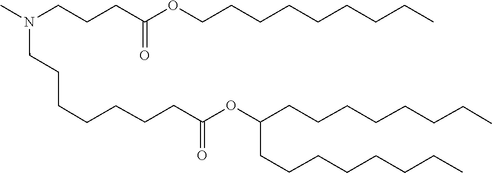 [SMILES: CCCCCCCCCOC(=O)CCCN(C)CCCCCCCC(=O)OC(CCCCCCCC)CCCCCCCC] |
| I 127 | 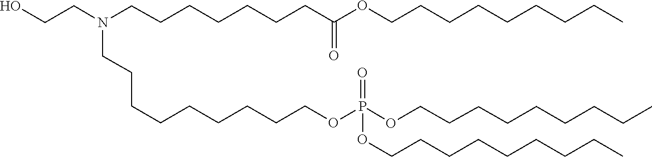 [SMILES: CCCCCCCCCOC(=O)CCCCCCCN(CCO)CCCCCCCCCOP(=O)(OCCCCCCCCC)OCCCCCCCCC] |
| I 128 | 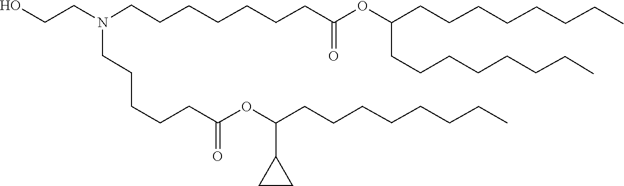 [SMILES: CCCCCCCCC(CCCCCCCC)OC(=O)CCCCCCCN(CCO)CCCCCC(=O)OC(CCCCCCCC)C1CC1] |
| I 129 | 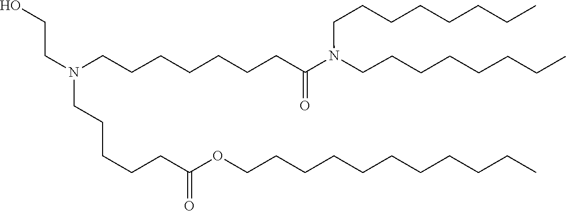 [SMILES: CCCCCCCCCCCOC(=O)CCCCCN(CCO)CCCCCCCC(=O)N(CCCCCCCC)CCCCCCCC] |
| I 130 | 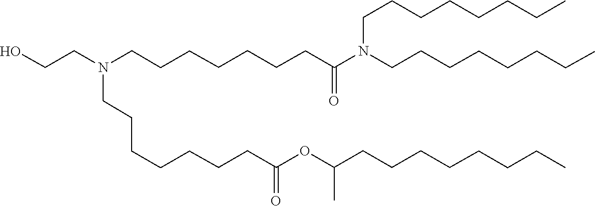 [SMILES: CCCCCCCCC(C)OC(=O)CCCCCCCN(CCO)CCCCCCCC(=O)N(CCCCCCCC)CCCCCCCC] |
| I 131 | 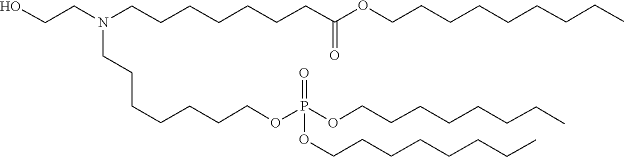 [SMILES: CCCCCCCCCOC(=O)CCCCCCCN(CCO)CCCCCCCOP(=O)(OCCCCCCCC)OCCCCCCCC] |
| I 132 | 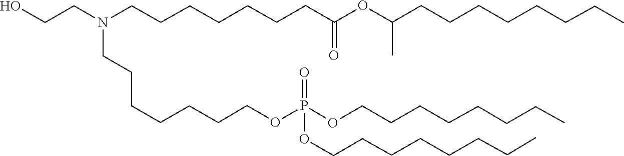 [SMILES: CCCCCCCCOP(=O)(OCCCCCCCC)OCCCCCCCN(CCO)CCCCCCCC(=O)OC(C)CCCCCCCC] |
| I 133 | 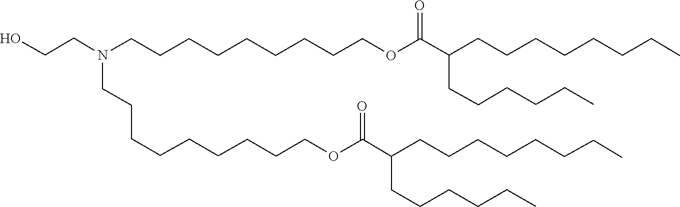 [SMILES: CCCCCCCCC(CCCCCC)C(=O)OCCCCCCCCCN(CCO)CCCCCCCCCOC(=O)C(CCCCCC)CCCCCCCC] |
| I 134 | 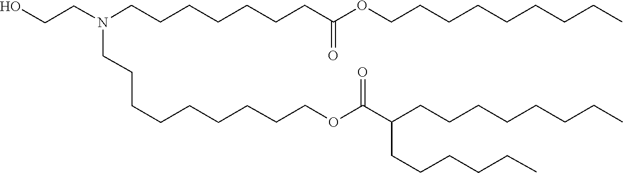 [SMILES: CCCCCCCCCOC(=O)CCCCCCCN(CCO)CCCCCCCCCOC(=O)C(CCCCCC)CCCCCCCC] |
| I 135 | 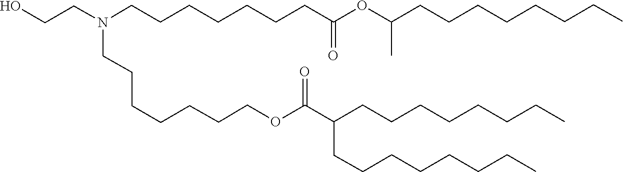 [SMILES: CCCCCCCCC(C)OC(=O)CCCCCCCN(CCO)CCCCCCCOC(=O)C(CCCCCCCC)CCCCCCCC] |
| I 136 | 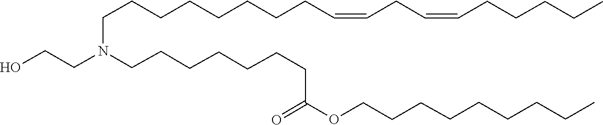 [SMILES: CCCCC/C=C\C/C=C\CCCCCCCCN(CCO)CCCCCCCC(=O)OCCCCCCCCC] |
| I 137 | 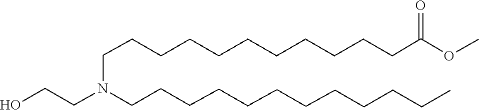 [SMILES: CCCCCCCCCCCCN(CCO)CCCCCCCCCCCC(=O)OC] |
| I 138 | 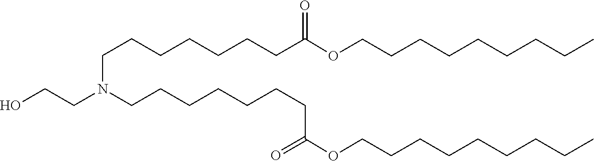 [SMILES: CCCCCCCCCOC(=O)CCCCCCCN(CCO)CCCCCCCC(=O)OCCCCCCCCC] |
| I 139 | 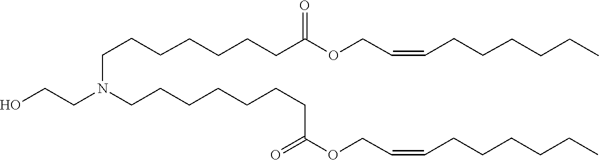 [SMILES: CCCCCC/C=C\COC(=O)CCCCCCCN(CCO)CCCCCCCC(=O)OC/C=C\CCCCCC] |
| I 140 | 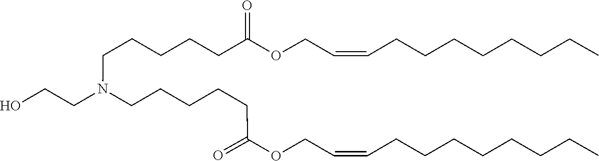 [SMILES: CCCCCCCC/C=C\COC(=O)CCCCCN(CCO)CCCCCC(=O)OC/C=C\CCCCCCCC] |
| I 141 | 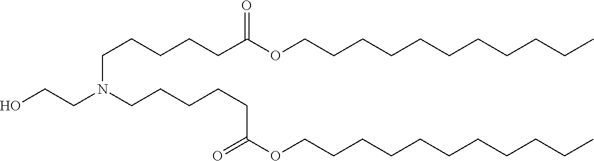 [SMILES: CCCCCCCCCCCOC(=O)CCCCCN(CCO)CCCCCC(=O)OCCCCCCCCCCC] |
| I 142 | 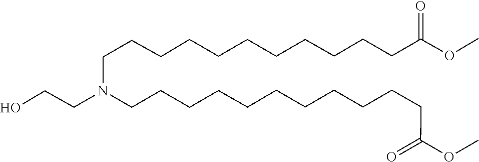 [SMILES: COC(=O)CCCCCCCCCCCN(CCO)CCCCCCCCCCCC(=O)OC] |
| I 143 | 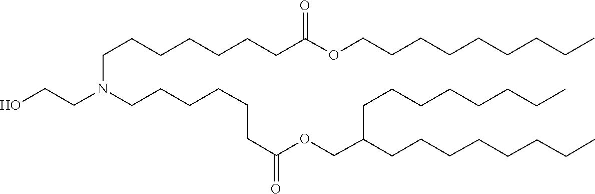 [SMILES: CCCCCCCCCOC(=O)CCCCCCCN(CCO)CCCCCCC(=O)OCC(CCCCCCCC)CCCCCCCC] |
| I 144 | 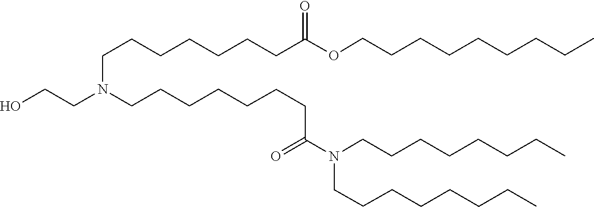 [SMILES: CCCCCCCCCOC(=O)CCCCCCCN(CCO)CCCCCCCC(=O)N(CCCCCCCC)CCCCCCCC] |
| I 145 | 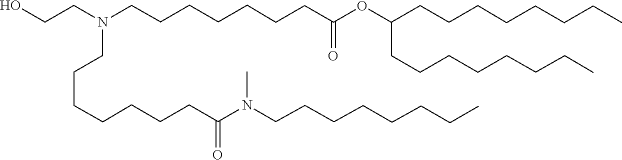 [SMILES: CCCCCCCCC(CCCCCCCC)OC(=O)CCCCCCCN(CCO)CCCCCCCC(=O)N(C)CCCCCCCC] |
| I 146 | 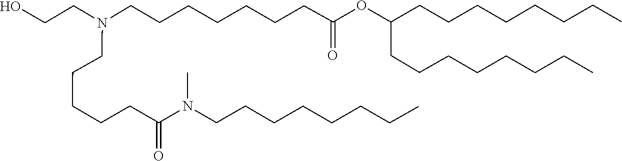 [SMILES: CCCCCCCCC(CCCCCCCC)OC(=O)CCCCCCCN(CCO)CCCCCC(=O)N(C)CCCCCCCC] |
| I 147 | 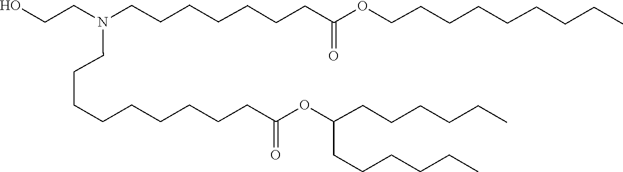 [SMILES: CCCCCCCCCOC(=O)CCCCCCCN(CCO)CCCCCCCCCC(=O)OC(CCCCCC)CCCCCC] |
| I 148 | 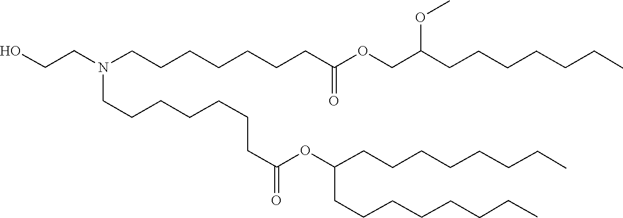 [SMILES: CCCCCCCCC(CCCCCCCC)OC(=O)CCCCCCCN(CCO)CCCCCCCC(=O)OCC(CCCCCCC)OC] |
| I 149 | 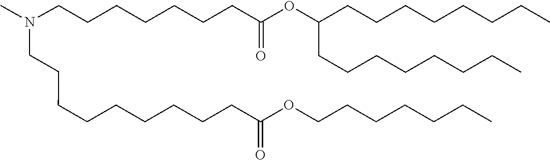 [SMILES: CCCCCCCCC(CCCCCCCC)OC(=O)CCCCCCCN(C)CCCCCCCCCC(=O)OCCCCCCC] |
| I 150 | 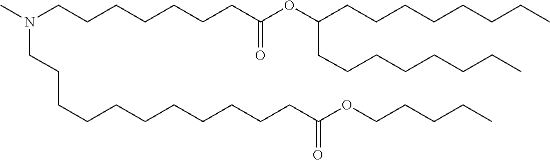 [SMILES: CCCCCCCCC(CCCCCCCC)OC(=O)CCCCCCCN(C)CCCCCCCCCCCC(=O)OCCCCC] |
| I 151 | 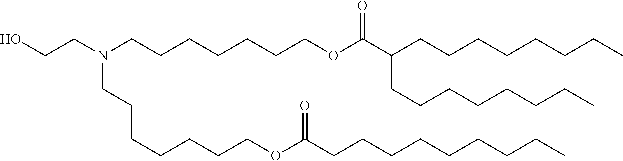 [SMILES: CCCCCCCCCC(=O)OCCCCCCCN(CCO)CCCCCCCOC(=O)C(CCCCCCCC)CCCCCCCC] |
| I 152 | 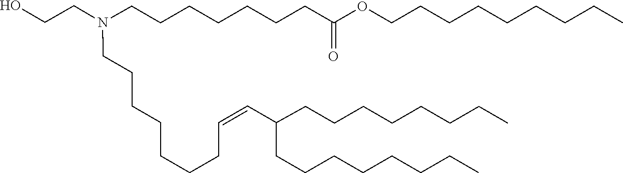 [SMILES: CCCCCCCCCOC(=O)CCCCCCCN(CCO)CCCCCCC/C=C\C(CCCCCCCC)CCCCCCCC] |
| I 153 | 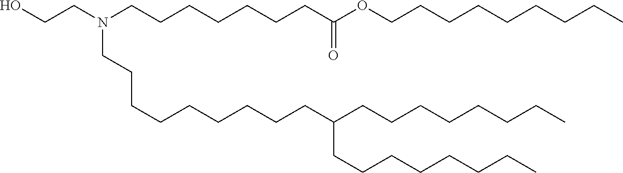 [SMILES: CCCCCCCCCOC(=O)CCCCCCCN(CCO)CCCCCCCCCC(CCCCCCCC)CCCCCCCC] |
| I 154 | 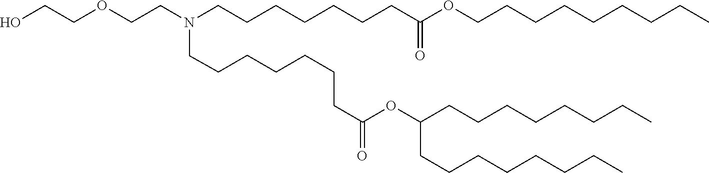 [SMILES: CCCCCCCCCOC(=O)CCCCCCCN(CCCCCCCC(=O)OC(CCCCCCCC)CCCCCCCC)CCOCCO] |
| I 155 | 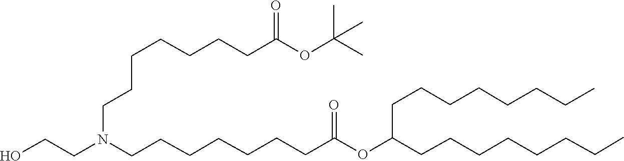 [SMILES: CCCCCCCCC(CCCCCCCC)OC(=O)CCCCCCCN(CCO)CCCCCCCC(=O)OC(C)(C)C] |
| I 156 | 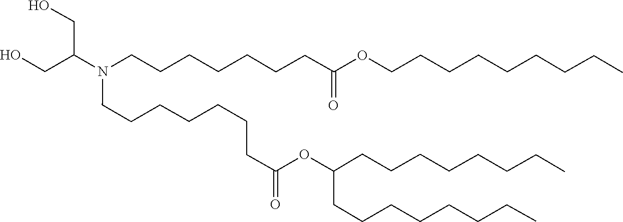 [SMILES: CCCCCCCCCOC(=O)CCCCCCCN(CCCCCCCC(=O)OC(CCCCCCCC)CCCCCCCC)C(CO)CO] |
| I 157 | 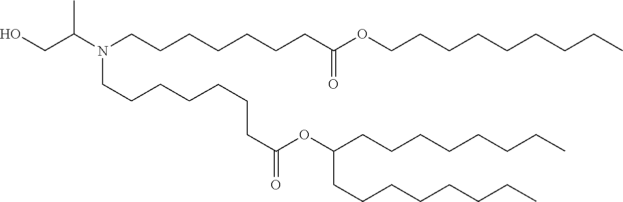 [SMILES: CCCCCCCCCOC(=O)CCCCCCCN(CCCCCCCC(=O)OC(CCCCCCCC)CCCCCCCC)C(C)CO] |
| I 158 | 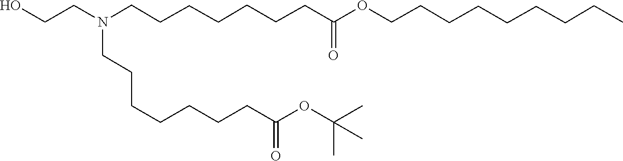 [SMILES: CCCCCCCCCOC(=O)CCCCCCCN(CCO)CCCCCCCC(=O)OC(C)(C)C] |
| I 159 | 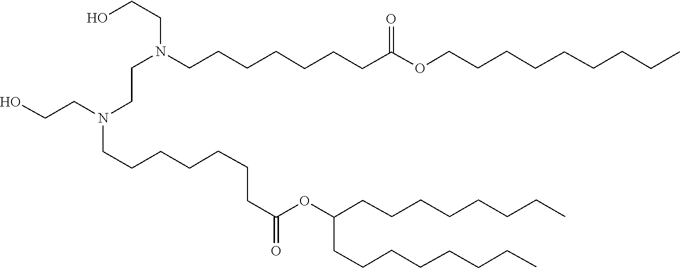 [SMILES: CCCCCCCCCOC(=O)CCCCCCCN(CCO)CCN(CCO)CCCCCCCC(=O)OC(CCCCCCCC)CCCCCCCC] |
| I 160 | 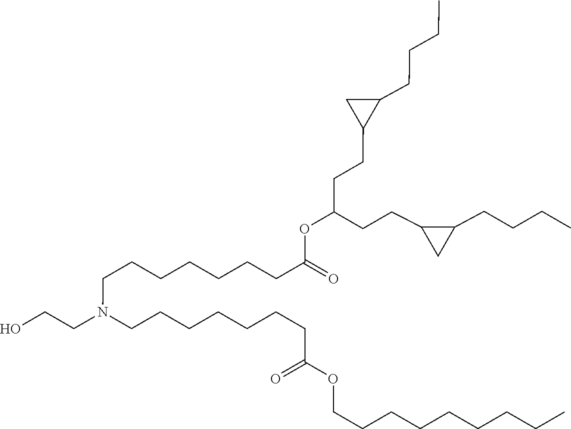 [SMILES: CCCCCCCCCOC(=O)CCCCCCCN(CCO)CCCCCCCC(=O)OC(CCC1CC1CCCC)CCC1CC1CCCC] |
| I 161 | 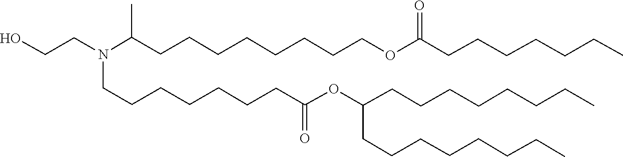 [SMILES: CCCCCCCCC(CCCCCCCC)OC(=O)CCCCCCCN(CCO)C(C)CCCCCCCCOC(=O)CCCCCCC] |
| I 162 | 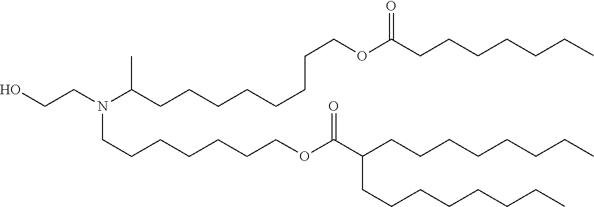 [SMILES: CCCCCCCCC(CCCCCCCC)C(=O)OCCCCCCCN(CCO)C(C)CCCCCCCCOC(=O)CCCCCCC] |
| I 163 | 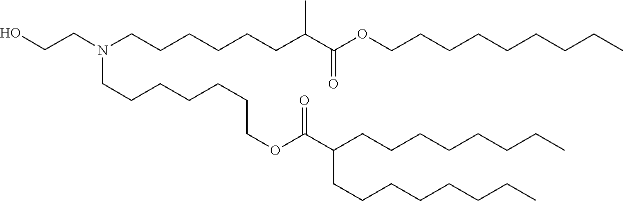 [SMILES: CCCCCCCCCOC(=O)C(C)CCCCCCN(CCO)CCCCCCCOC(=O)C(CCCCCCCC)CCCCCCCC] |
| I 164 | 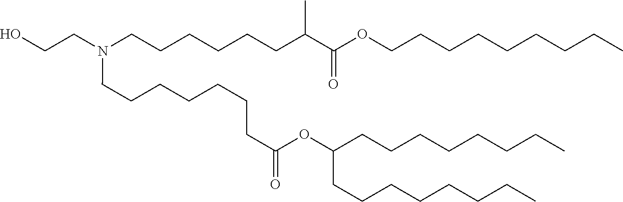 [SMILES: CCCCCCCCCOC(=O)C(C)CCCCCCN(CCO)CCCCCCCC(=O)OC(CCCCCCCC)CCCCCCCC] |
| I 165 | 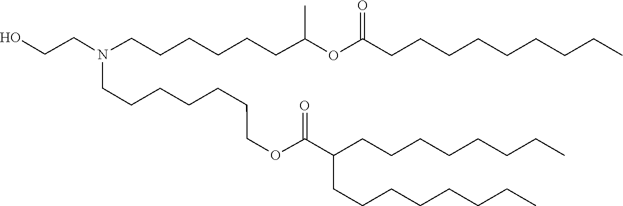 [SMILES: CCCCCCCCCC(=O)OC(C)CCCCCCN(CCO)CCCCCCCOC(=O)C(CCCCCCCC)CCCCCCCC] |
| I 166 | 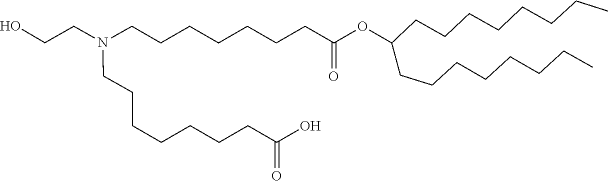 [SMILES: CCCCCCCCC(CCCCCCCC)OC(=O)CCCCCCCN(CCO)CCCCCCCC(=O)O] |
| I 167 | 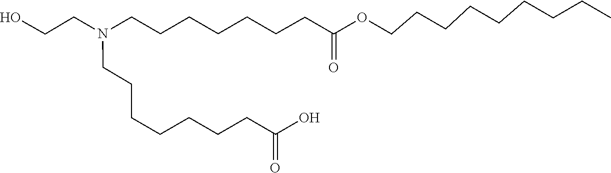 [SMILES: CCCCCCCCCOC(=O)CCCCCCCN(CCO)CCCCCCCC(=O)O] |
| I 168 | 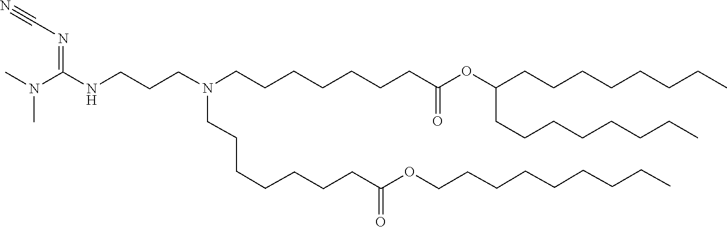 [SMILES: CCCCCCCCCOC(=O)CCCCCCCN(CCCCCCCC(=O)OC(CCCCCCCC)CCCCCCCC)CCCN/C(=N/C#N)N(C)C] |
| I 169 | 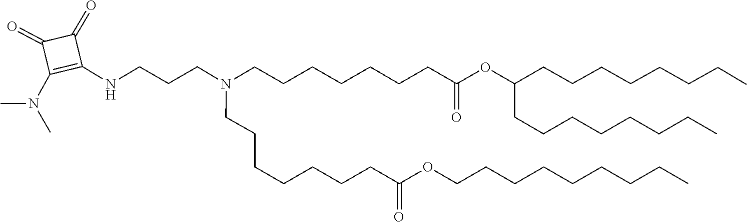 [SMILES: CCCCCCCCCOC(=O)CCCCCCCN(CCCCCCCC(=O)OC(CCCCCCCC)CCCCCCCC)CCCNc1c(N(C)C)c(=O)c1=O] |
| I 170 | 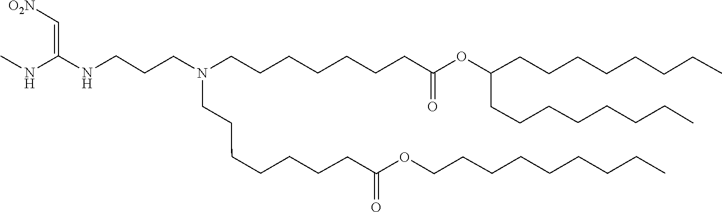 [SMILES: CCCCCCCCCOC(=O)CCCCCCCN(CCCCCCCC(=O)OC(CCCCCCCC)CCCCCCCC)CCCN/C(=C/[N+](=O)[O-])NC] |
| I 171 | 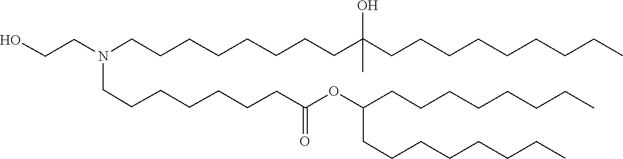 [SMILES: CCCCCCCCCC(C)(O)CCCCCCCCN(CCO)CCCCCCCC(=O)OC(CCCCCCCC)CCCCCCCC] |
| I 172 | 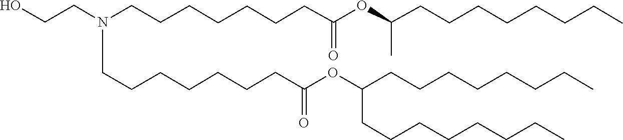 [SMILES: CCCCCCCCC(CCCCCCCC)OC(=O)CCCCCCCN(CCO)CCCCCCCC(=O)O[C@H](C)CCCCCCCC] |
| I 173 | 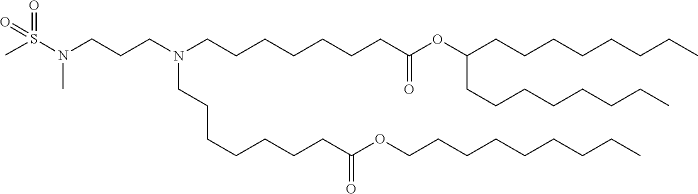 [SMILES: CCCCCCCCCOC(=O)CCCCCCCN(CCCCCCCC(=O)OC(CCCCCCCC)CCCCCCCC)CCCN(C)S(C)(=O)=O] |
| I 174 | 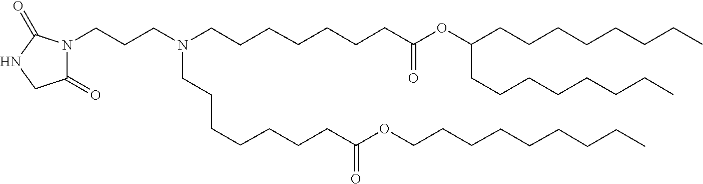 [SMILES: CCCCCCCCCOC(=O)CCCCCCCN(CCCCCCCC(=O)OC(CCCCCCCC)CCCCCCCC)CCCN1C(=O)CNC1=O] |
| I 175 | 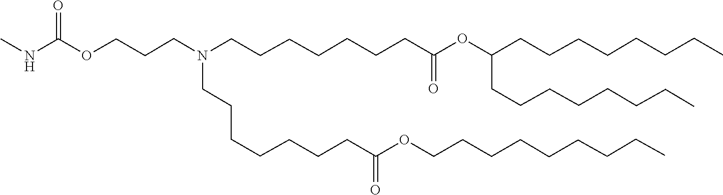 [SMILES: CCCCCCCCCOC(=O)CCCCCCCN(CCCCCCCC(=O)OC(CCCCCCCC)CCCCCCCC)CCCOC(=O)NC] |
| I 176 | 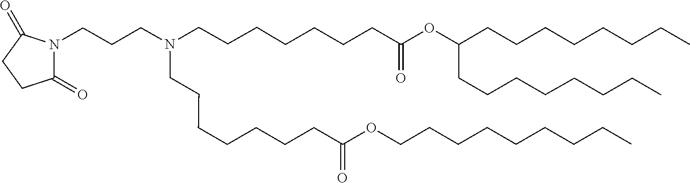 [SMILES: CCCCCCCCCOC(=O)CCCCCCCN(CCCCCCCC(=O)OC(CCCCCCCC)CCCCCCCC)CCCN1C(=O)CCC1=O] |
| I 177 | 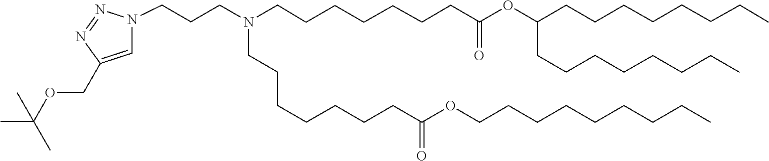 [SMILES: CCCCCCCCCOC(=O)CCCCCCCN(CCCCCCCC(=O)OC(CCCCCCCC)CCCCCCCC)CCCn1cc(COC(C)(C)C)nn1] |
| I 178 | 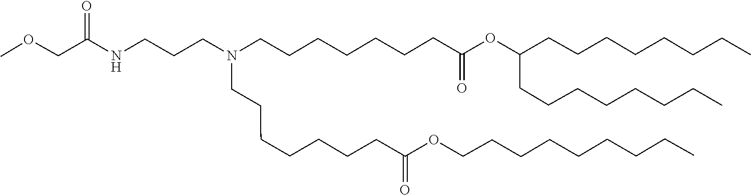 [SMILES: CCCCCCCCCOC(=O)CCCCCCCN(CCCCCCCC(=O)OC(CCCCCCCC)CCCCCCCC)CCCNC(=O)COC] |
| I 179 | 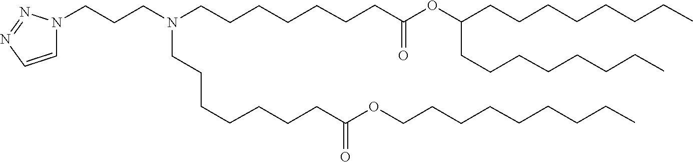 [SMILES: CCCCCCCCCOC(=O)CCCCCCCN(CCCCCCCC(=O)OC(CCCCCCCC)CCCCCCCC)CCCn1ccnn1] |
| I 180 | 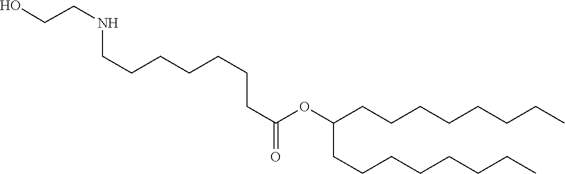 [SMILES: CCCCCCCCC(CCCCCCCC)OC(=O)CCCCCCCNCCO] |
| I 181 | 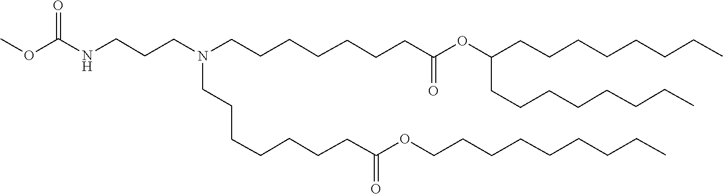 [SMILES: CCCCCCCCCOC(=O)CCCCCCCN(CCCCCCCC(=O)OC(CCCCCCCC)CCCCCCCC)CCCNC(=O)OC] |
| I 182 | 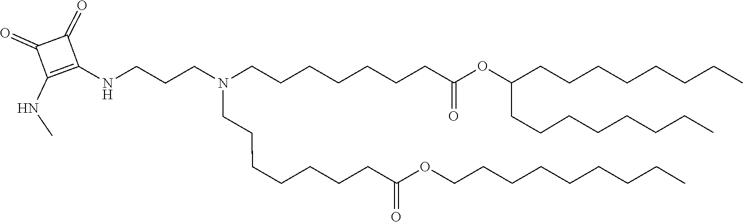 [SMILES: CCCCCCCCCOC(=O)CCCCCCCN(CCCCCCCC(=O)OC(CCCCCCCC)CCCCCCCC)CCCNc1c(NC)c(=O)c1=O] |
| I 183 | 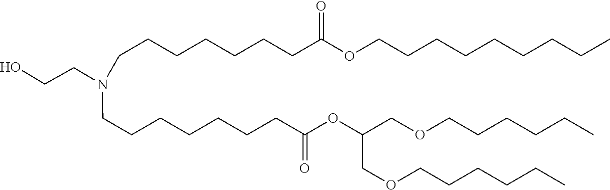 [SMILES: CCCCCCCCCOC(=O)CCCCCCCN(CCO)CCCCCCCC(=O)OC(COCCCCCC)COCCCCCC] |
| I 184 | 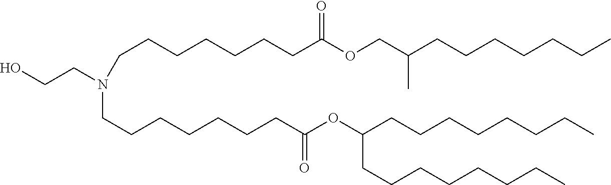 [SMILES: CCCCCCCCC(CCCCCCCC)OC(=O)CCCCCCCN(CCO)CCCCCCCC(=O)OCC(C)CCCCCCC] |
| I 185 | 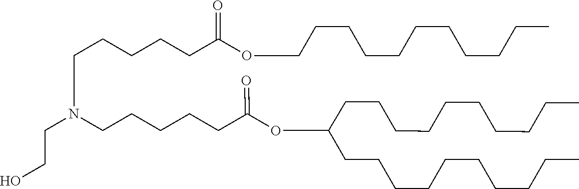 [SMILES: CCCCCCCCCCCOC(=O)CCCCCN(CCO)CCCCCC(=O)OC(CCCCCCCCCC)CCCCCCCCCC] |
| I 186 | 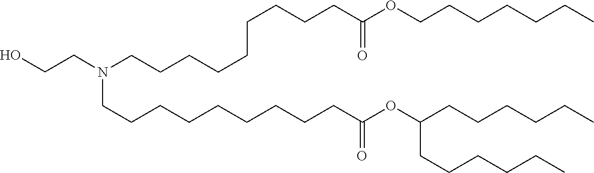 [SMILES: CCCCCCCOC(=O)CCCCCCCCCN(CCO)CCCCCCCCCC(=O)OC(CCCCCC)CCCCCC] |
| I 187 | 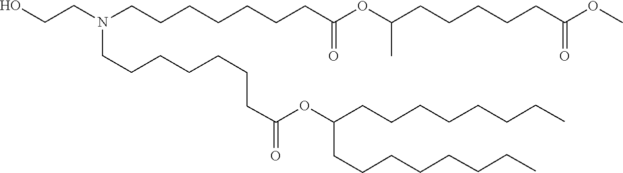 [SMILES: CCCCCCCCC(CCCCCCCC)OC(=O)CCCCCCCN(CCO)CCCCCCCC(=O)OC(C)CCCCCC(=O)OC] |
| I 188 | 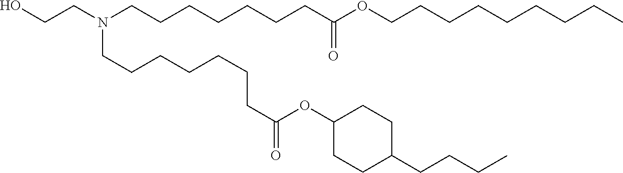 [SMILES: CCCCCCCCCOC(=O)CCCCCCCN(CCO)CCCCCCCC(=O)OC1CCC(CCCC)CC1] |
| I 189 | 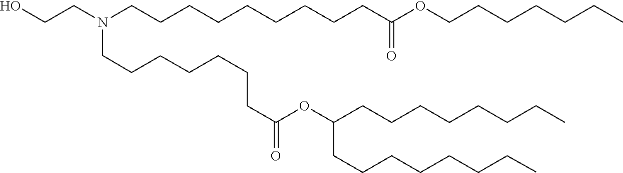 [SMILES: CCCCCCCCC(CCCCCCCC)OC(=O)CCCCCCCN(CCO)CCCCCCCCCC(=O)OCCCCCCC] |
| I 190 | 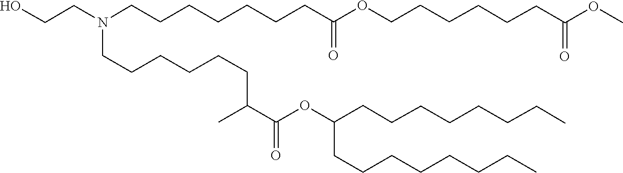 [SMILES: CCCCCCCCC(CCCCCCCC)OC(=O)C(C)CCCCCCN(CCO)CCCCCCCC(=O)OCCCCCCC(=O)OC] |
| I 191 | 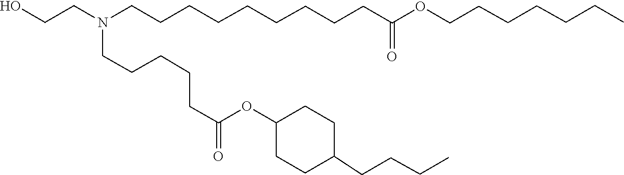 [SMILES: CCCCCCCOC(=O)CCCCCCCCCN(CCO)CCCCCC(=O)OC1CCC(CCCC)CC1] |
| I 192 | 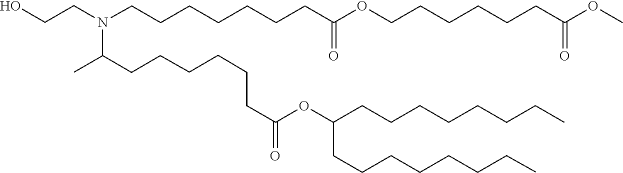 [SMILES: CCCCCCCCC(CCCCCCCC)OC(=O)CCCCCCC(C)N(CCO)CCCCCCCC(=O)OCCCCCCC(=O)OC] |
| I 193 | 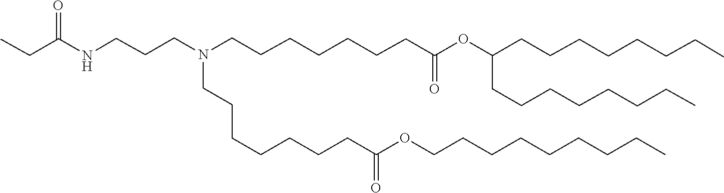 [SMILES: CCCCCCCCCOC(=O)CCCCCCCN(CCCCCCCC(=O)OC(CCCCCCCC)CCCCCCCC)CCCNC(=O)CC] |
| I 194 | 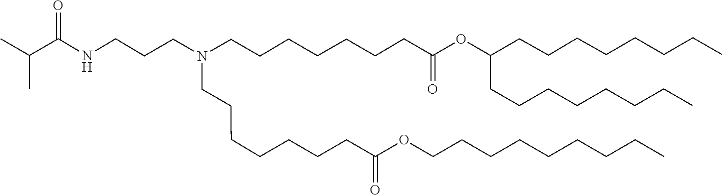 [SMILES: CCCCCCCCCOC(=O)CCCCCCCN(CCCCCCCC(=O)OC(CCCCCCCC)CCCCCCCC)CCCNC(=O)C(C)C] |
| I 195 | 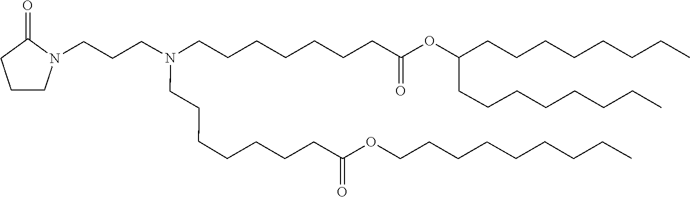 [SMILES: CCCCCCCCCOC(=O)CCCCCCCN(CCCCCCCC(=O)OC(CCCCCCCC)CCCCCCCC)CCCN1CCCC1=O] |
| I 196 | 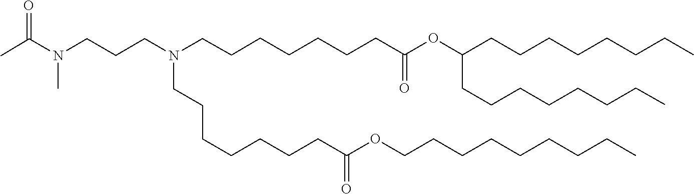 [SMILES: CCCCCCCCCOC(=O)CCCCCCCN(CCCCCCCC(=O)OC(CCCCCCCC)CCCCCCCC)CCCN(C)C(C)=O] |
| I 197 | 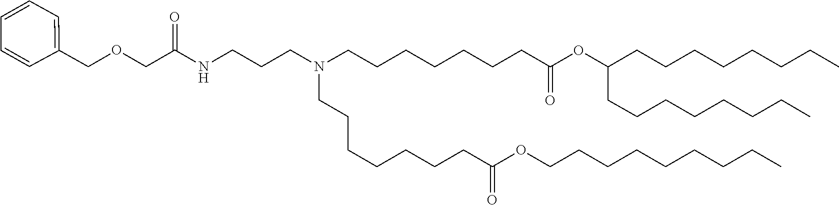 [SMILES: CCCCCCCCCOC(=O)CCCCCCCN(CCCCCCCC(=O)OC(CCCCCCCC)CCCCCCCC)CCCNC(=O)COCc1ccccc1] |
| I 198 | 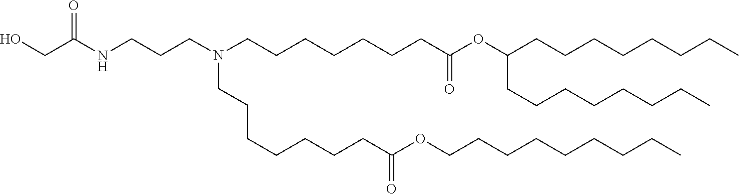 [SMILES: CCCCCCCCCOC(=O)CCCCCCCN(CCCCCCCC(=O)OC(CCCCCCCC)CCCCCCCC)CCCNC(=O)CO] |
| I 199 | 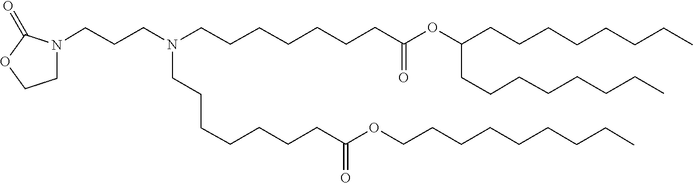 [SMILES: CCCCCCCCCOC(=O)CCCCCCCN(CCCCCCCC(=O)OC(CCCCCCCC)CCCCCCCC)CCCN1CCOC1=O] |
| I 200 | 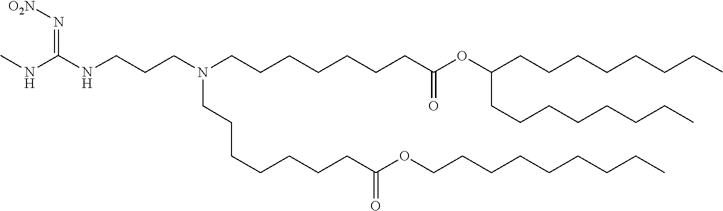 [SMILES: CCCCCCCCCOC(=O)CCCCCCCN(CCCCCCCC(=O)OC(CCCCCCCC)CCCCCCCC)CCCN/C(=N/[N+](=O)[O-])NC] |
| I 201 | 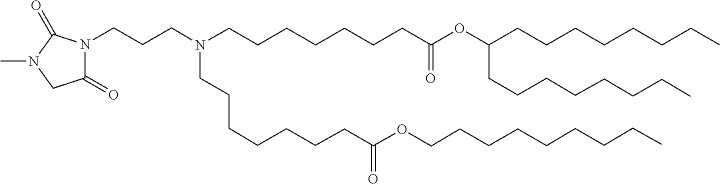 [SMILES: CCCCCCCCCOC(=O)CCCCCCCN(CCCCCCCC(=O)OC(CCCCCCCC)CCCCCCCC)CCCN1C(=O)CN(C)C1=O] |
| I 202 | 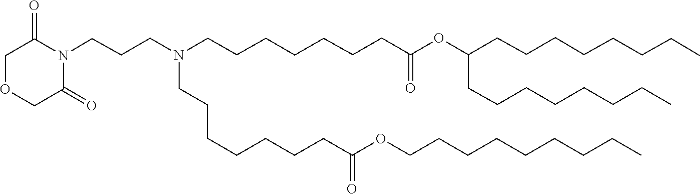 [SMILES: CCCCCCCCCOC(=O)CCCCCCCN(CCCCCCCC(=O)OC(CCCCCCCC)CCCCCCCC)CCCN1C(=O)COCC1=O] |
| I 203 | 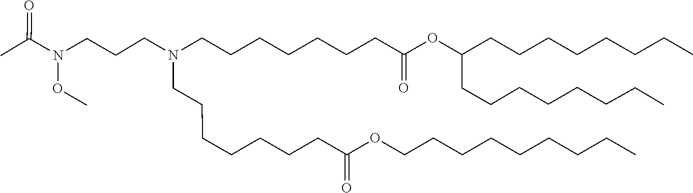 [SMILES: CCCCCCCCCOC(=O)CCCCCCCN(CCCCCCCC(=O)OC(CCCCCCCC)CCCCCCCC)CCCN(OC)C(C)=O] |
| I 204 | 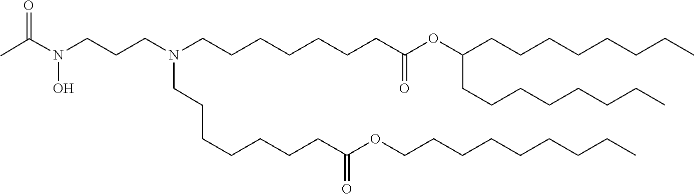 [SMILES: CCCCCCCCCOC(=O)CCCCCCCN(CCCCCCCC(=O)OC(CCCCCCCC)CCCCCCCC)CCCN(O)C(C)=O] |
| I 205 | 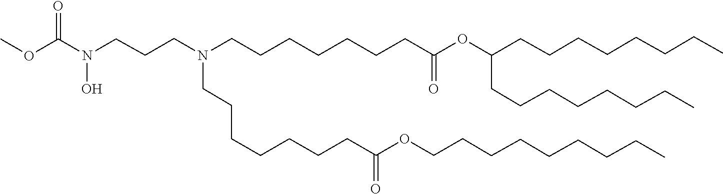 [SMILES: CCCCCCCCCOC(=O)CCCCCCCN(CCCCCCCC(=O)OC(CCCCCCCC)CCCCCCCC)CCCN(O)C(=O)OC] |
| I 206 | 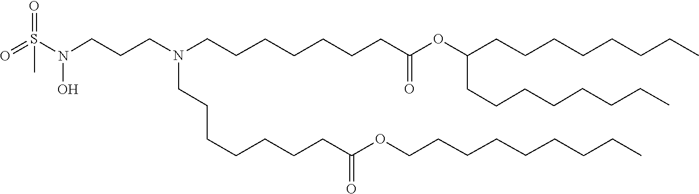 [SMILES: CCCCCCCCCOC(=O)CCCCCCCN(CCCCCCCC(=O)OC(CCCCCCCC)CCCCCCCC)CCCN(O)S(C)(=O)=O] |
| I 207 | 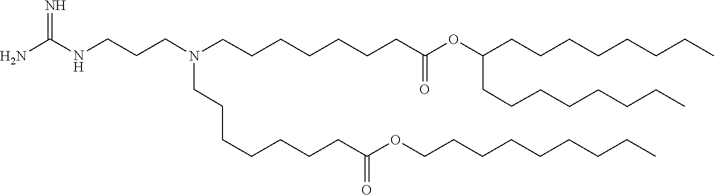 [SMILES: CCCCCCCCCOC(=O)CCCCCCCN(CCCCCCCC(=O)OC(CCCCCCCC)CCCCCCCC)CCCNC(=N)N] |
| I 208 | 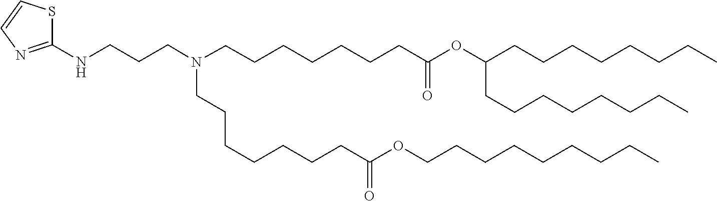 [SMILES: CCCCCCCCCOC(=O)CCCCCCCN(CCCCCCCC(=O)OC(CCCCCCCC)CCCCCCCC)CCCNc1nccs1] |
| I 209 | 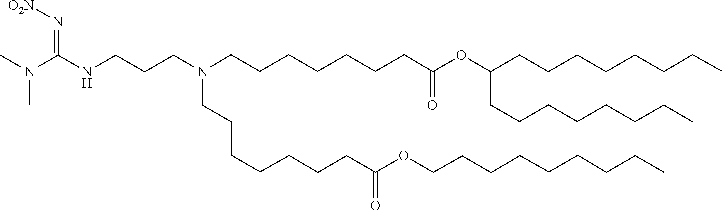 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I 210 | 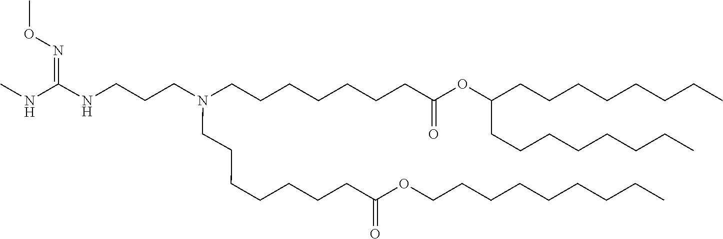 [SMILES: CCCCCCCCCOC(=O)CCCCCCCN(CCCCCCCC(=O)OC(CCCCCCCC)CCCCCCCC)CCCN/C(=N/OC)NC] |
| I 211 | 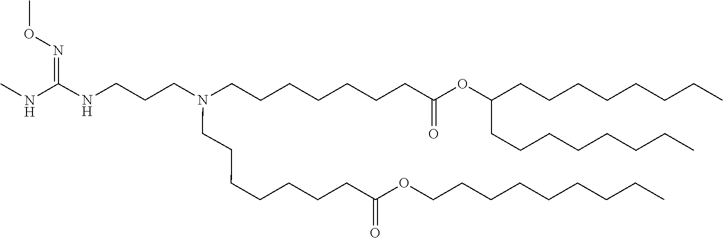 [SMILES: CCCCCCCCCOC(=O)CCCCCCCN(CCCCCCCC(=O)OC(CCCCCCCC)CCCCCCCC)CCCN/C(=N/OC)NC] |
| I 212 | 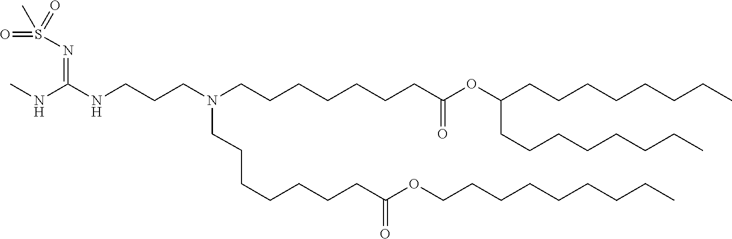 [SMILES: CCCCCCCCCOC(=O)CCCCCCCN(CCCCCCCC(=O)OC(CCCCCCCC)CCCCCCCC)CCCN/C(=N/S(C)(=O)=O)NC] |
| I 213 | 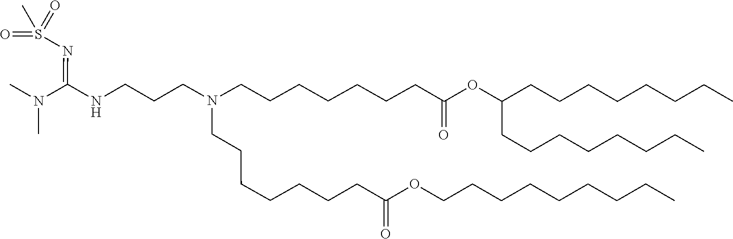 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I 214 | 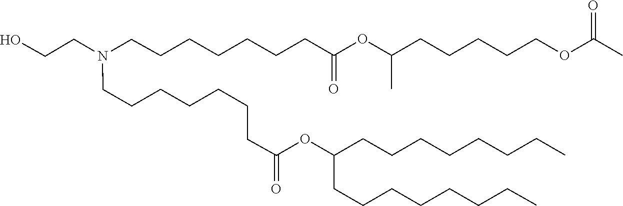 [SMILES: CCCCCCCCC(CCCCCCCC)OC(=O)CCCCCCCN(CCO)CCCCCCCC(=O)OC(C)CCCCCOC(C)=O] |
| I 215 | 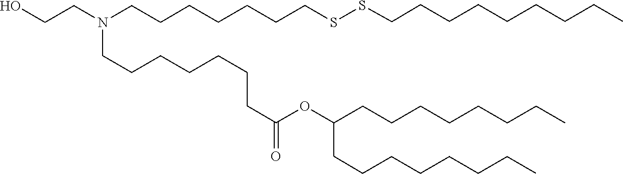 [SMILES: CCCCCCCCCSSCCCCCCCN(CCO)CCCCCCCC(=O)OC(CCCCCCCC)CCCCCCCC] |
| I 216 | 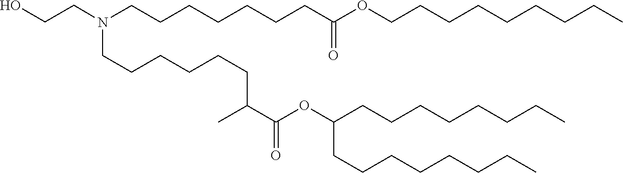 [SMILES: CCCCCCCCCOC(=O)CCCCCCCN(CCO)CCCCCCC(C)C(=O)OC(CCCCCCCC)CCCCCCCC] |
| I 217 | 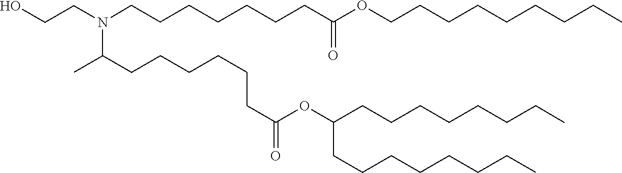 [SMILES: CCCCCCCCCOC(=O)CCCCCCCN(CCO)C(C)CCCCCCC(=O)OC(CCCCCCCC)CCCCCCCC] |
| I 218 | 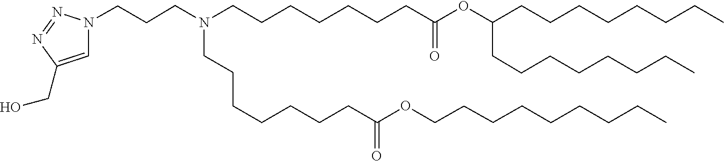 [SMILES: CCCCCCCCCOC(=O)CCCCCCCN(CCCCCCCC(=O)OC(CCCCCCCC)CCCCCCCC)CCCn1cc(CO)nn1] |
| I 219 | 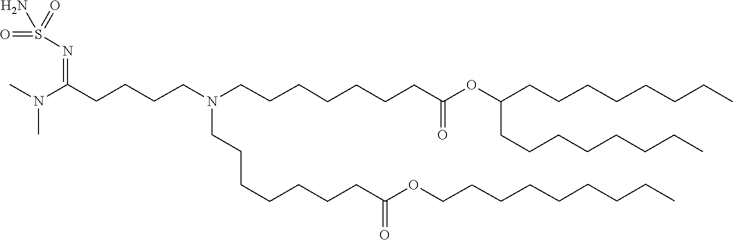 [SMILES: CCCCCCCCCOC(=O)CCCCCCCN(CCCCCCCC(=O)OC(CCCCCCCC)CCCCCCCC)CCCC/C(=N/S(N)(=O)=O)N(C)C] |
| I 220 | 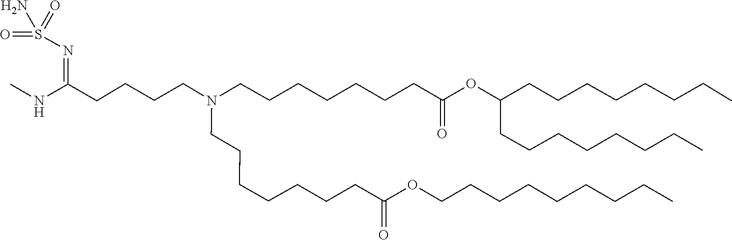 [SMILES: CCCCCCCCCOC(=O)CCCCCCCN(CCCCCCCC(=O)OC(CCCCCCCC)CCCCCCCC)CCCC/C(=N/S(N)(=O)=O)NC] |
| I 221 | 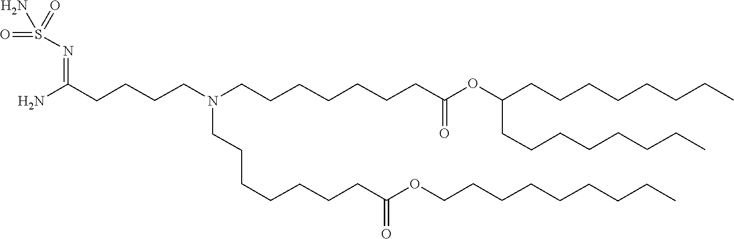 [SMILES: CCCCCCCCCOC(=O)CCCCCCCN(CCCCCCCC(=O)OC(CCCCCCCC)CCCCCCCC)CCCC/C(N)=N/S(N)(=O)=O] |
| I 222 | 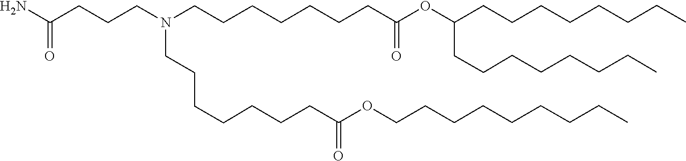 [SMILES: CCCCCCCCCOC(=O)CCCCCCCN(CCCCCCCC(=O)OC(CCCCCCCC)CCCCCCCC)CCCC(N)=O] |
| I 223 | 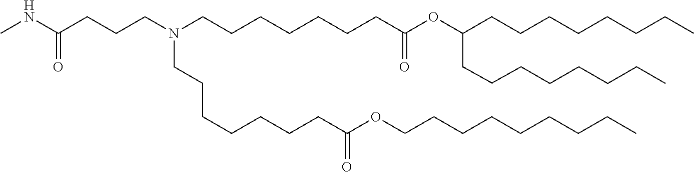 [SMILES: CCCCCCCCCOC(=O)CCCCCCCN(CCCCCCCC(=O)OC(CCCCCCCC)CCCCCCCC)CCCC(=O)NC] |
| I 224 | 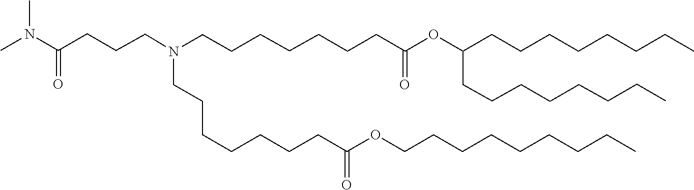 [SMILES: CCCCCCCCCOC(=O)CCCCCCCN(CCCCCCCC(=O)OC(CCCCCCCC)CCCCCCCC)CCCC(=O)N(C)C] |
| I 225 | 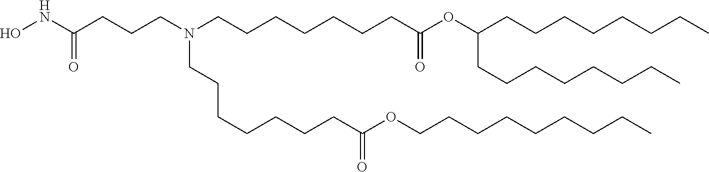 [SMILES: CCCCCCCCCOC(=O)CCCCCCCN(CCCCCCCC(=O)OC(CCCCCCCC)CCCCCCCC)CCCC(=O)NO] |
| I 226 | 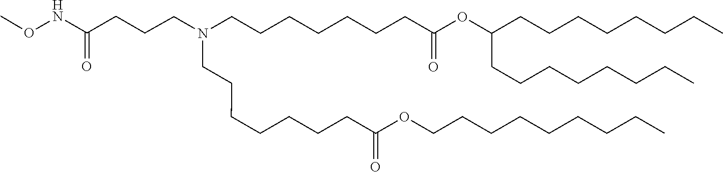 [SMILES: CCCCCCCCCOC(=O)CCCCCCCN(CCCCCCCC(=O)OC(CCCCCCCC)CCCCCCCC)CCCC(=O)NOC] |
| I 227 | 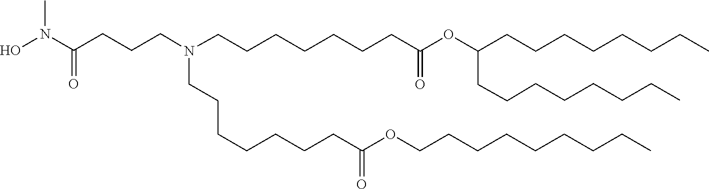 [SMILES: CCCCCCCCCOC(=O)CCCCCCCN(CCCCCCCC(=O)OC(CCCCCCCC)CCCCCCCC)CCCC(=O)N(C)O] |
| I 228 | 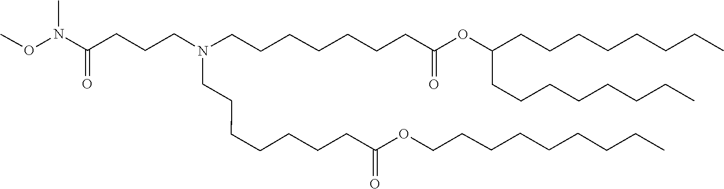 [SMILES: CCCCCCCCCOC(=O)CCCCCCCN(CCCCCCCC(=O)OC(CCCCCCCC)CCCCCCCC)CCCC(=O)N(C)OC] |
| I 229 | 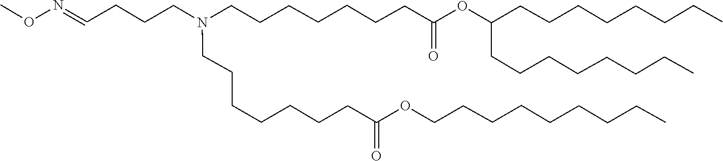 [SMILES: CCCCCCCCCOC(=O)CCCCCCCN(CCC/C=N/OC)CCCCCCCC(=O)OC(CCCCCCCC)CCCCCCCC] |
| I 230 | 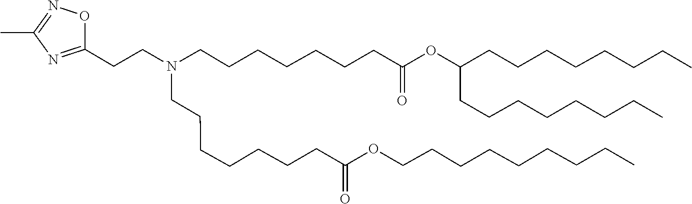 [SMILES: CCCCCCCCCOC(=O)CCCCCCCN(CCCCCCCC(=O)OC(CCCCCCCC)CCCCCCCC)CCc1nc(C)no1] |
| I 231 | 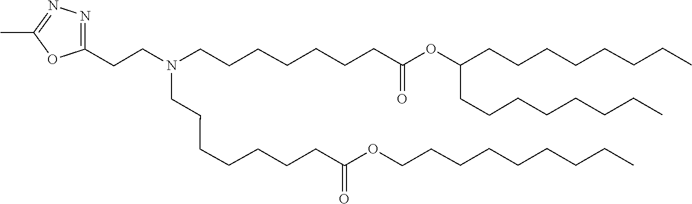 [SMILES: CCCCCCCCCOC(=O)CCCCCCCN(CCCCCCCC(=O)OC(CCCCCCCC)CCCCCCCC)CCc1nnc(C)o1] |
| I 232 | 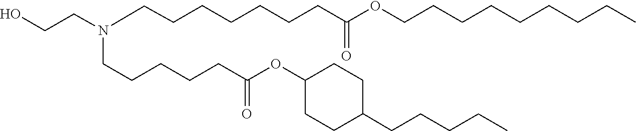 [SMILES: CCCCCCCCCOC(=O)CCCCCCCN(CCO)CCCCCC(=O)OC1CCC(CCCCC)CC1] |
| I 233 | 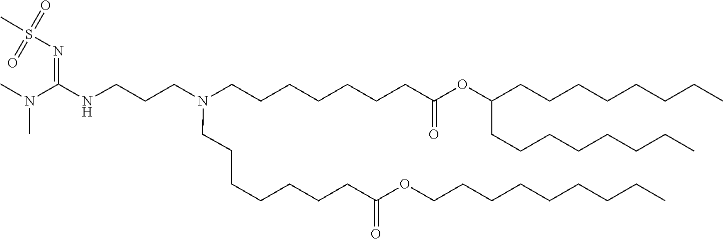 [SMILES: CCCCCCCCCOC(=O)CCCCCCCN(CCCCCCCC(=O)OC(CCCCCCCC)CCCCCCCC)CCCN/C(=N/S(C)(=O)=O)N(C)C] |
| I 234 | 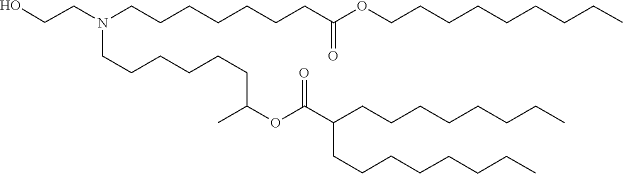 [SMILES: CCCCCCCCCOC(=O)CCCCCCCN(CCO)CCCCCCC(C)OC(=O)C(CCCCCCCC)CCCCCCCC] |
| I 235 | 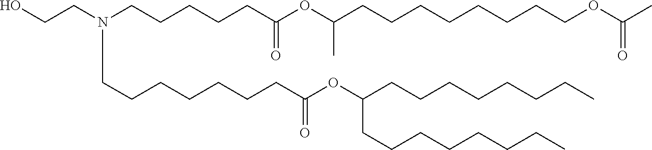 [SMILES: CCCCCCCCC(CCCCCCCC)OC(=O)CCCCCCCN(CCO)CCCCCC(=O)OC(C)CCCCCCCCOC(C)=O] |
| I 236 | 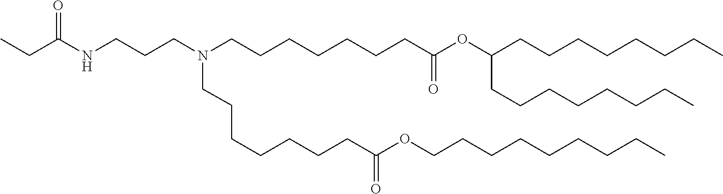 [SMILES: CCCCCCCCCOC(=O)CCCCCCCN(CCCCCCCC(=O)OC(CCCCCCCC)CCCCCCCC)CCCNC(=O)CC] |
| I 237 | 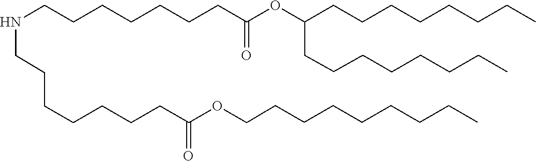 [SMILES: CCCCCCCCCOC(=O)CCCCCCCNCCCCCCCC(=O)OC(CCCCCCCC)CCCCCCCC] |
| I 238 | 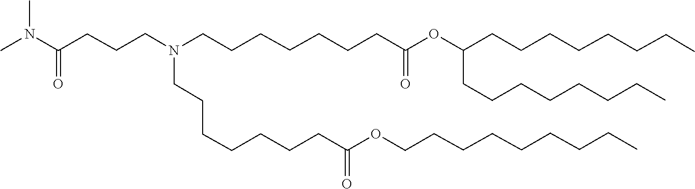 [SMILES: CCCCCCCCCOC(=O)CCCCCCCN(CCCCCCCC(=O)OC(CCCCCCCC)CCCCCCCC)CCCC(=O)N(C)C] |
| I 239 | 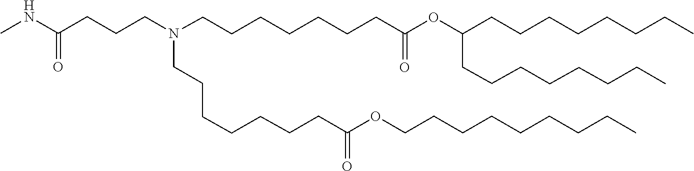 [SMILES: CCCCCCCCCOC(=O)CCCCCCCN(CCCCCCCC(=O)OC(CCCCCCCC)CCCCCCCC)CCCC(=O)NC] |
| I 240 | 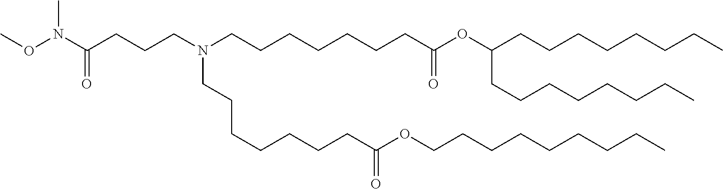 [SMILES: CCCCCCCCCOC(=O)CCCCCCCN(CCCCCCCC(=O)OC(CCCCCCCC)CCCCCCCC)CCCC(=O)N(C)OC] |
| I 241 | 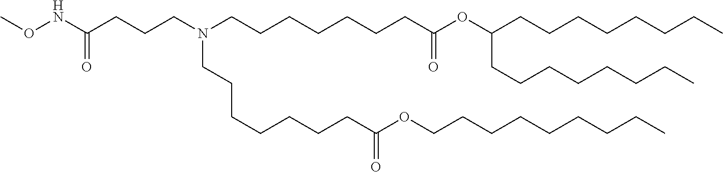 [SMILES: CCCCCCCCCOC(=O)CCCCCCCN(CCCCCCCC(=O)OC(CCCCCCCC)CCCCCCCC)CCCC(=O)NOC] |
| I 242 | 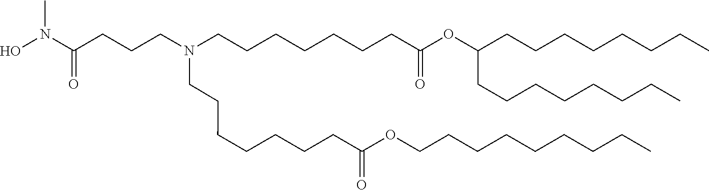 [SMILES: CCCCCCCCCOC(=O)CCCCCCCN(CCCCCCCC(=O)OC(CCCCCCCC)CCCCCCCC)CCCC(=O)N(C)O] |
| I 243 | 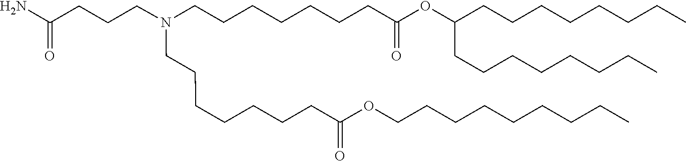 [SMILES: CCCCCCCCCOC(=O)CCCCCCCN(CCCCCCCC(=O)OC(CCCCCCCC)CCCCCCCC)CCCC(N)=O] |
| I 244 | 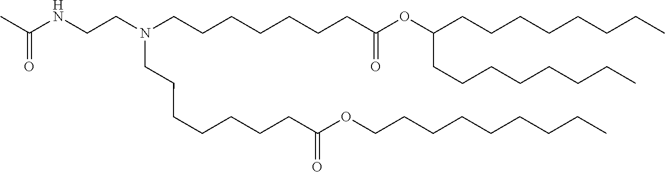 [SMILES: CCCCCCCCCOC(=O)CCCCCCCN(CCCCCCCC(=O)OC(CCCCCCCC)CCCCCCCC)CCNC(C)=O] |
| I 245 | 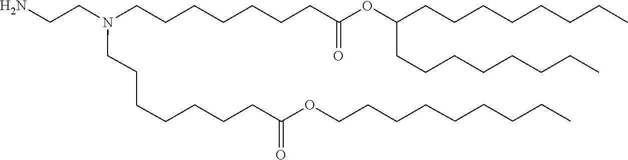 [SMILES: CCCCCCCCCOC(=O)CCCCCCCN(CCN)CCCCCCCC(=O)OC(CCCCCCCC)CCCCCCCC] |
| I 246 | 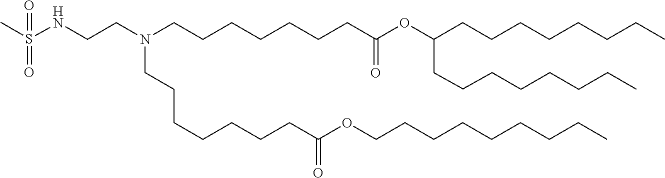 [SMILES: CCCCCCCCCOC(=O)CCCCCCCN(CCCCCCCC(=O)OC(CCCCCCCC)CCCCCCCC)CCNS(C)(=O)=O] |
| I 247 | 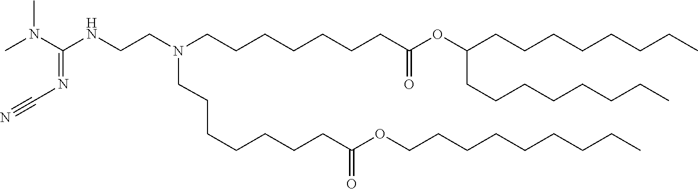 [SMILES: CCCCCCCCCOC(=O)CCCCCCCN(CCCCCCCC(=O)OC(CCCCCCCC)CCCCCCCC)CCN/C(=N/C#N)N(C)C] |
| I 248 | 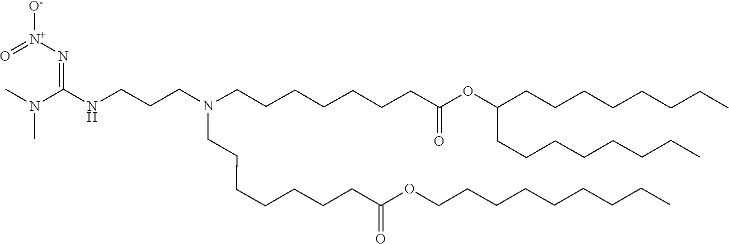 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I 249 | 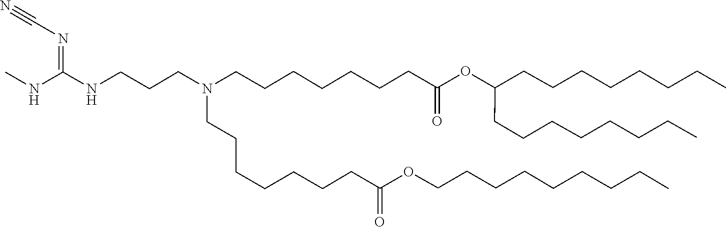 [SMILES: CCCCCCCCCOC(=O)CCCCCCCN(CCCCCCCC(=O)OC(CCCCCCCC)CCCCCCCC)CCCN/C(=N/C#N)NC] |
| I 250 | 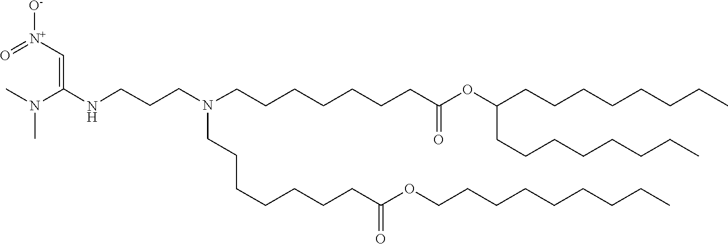 [SMILES: CCCCCCCCCOC(=O)CCCCCCCN(CCCCCCCC(=O)OC(CCCCCCCC)CCCCCCCC)CCCN/C(=C/[N+](=O)[O-])N(C)C] |
| I 251 | 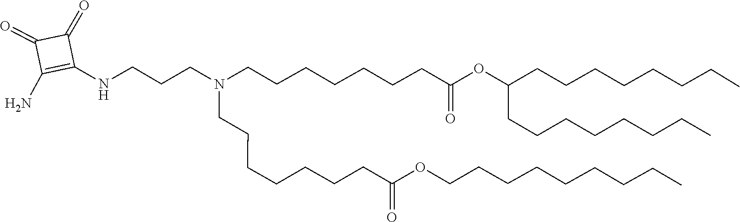 [SMILES: CCCCCCCCCOC(=O)CCCCCCCN(CCCCCCCC(=O)OC(CCCCCCCC)CCCCCCCC)CCCNc1c(N)c(=O)c1=O] |
| I 252 | 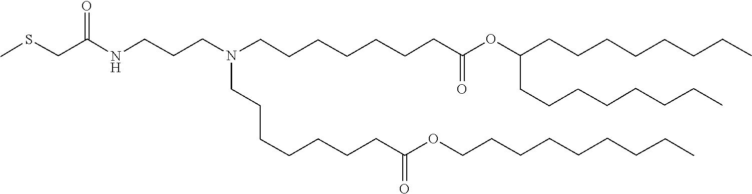 [SMILES: CCCCCCCCCOC(=O)CCCCCCCN(CCCCCCCC(=O)OC(CCCCCCCC)CCCCCCCC)CCCNC(=O)CSC] |
| I 253 | 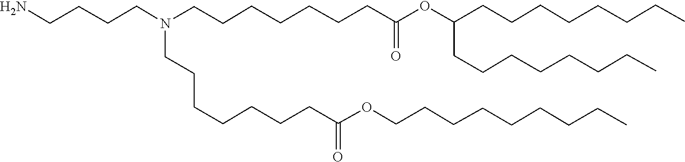 [SMILES: CCCCCCCCCOC(=O)CCCCCCCN(CCCCN)CCCCCCCC(=O)OC(CCCCCCCC)CCCCCCCC] |
| I 254 | 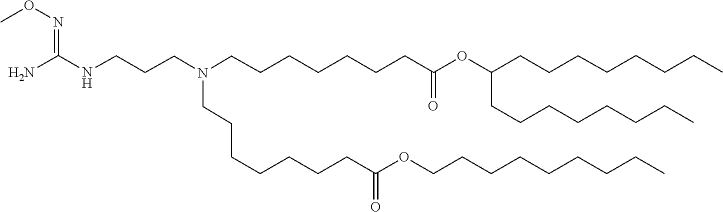 [SMILES: CCCCCCCCCOC(=O)CCCCCCCN(CCCCCCCC(=O)OC(CCCCCCCC)CCCCCCCC)CCCN/C(N)=N/OC] |
| I 255 | 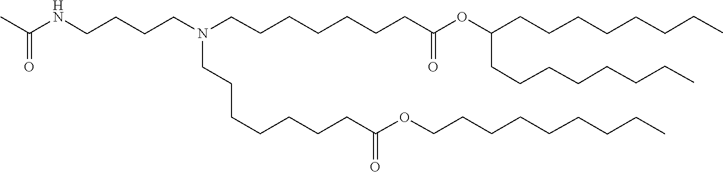 [SMILES: CCCCCCCCCOC(=O)CCCCCCCN(CCCCCCCC(=O)OC(CCCCCCCC)CCCCCCCC)CCCCNC(C)=O] |
| I 256 | 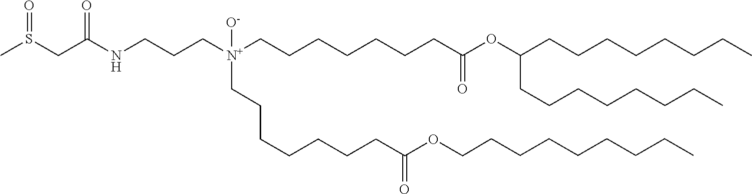 [SMILES: CCCCCCCCCOC(=O)CCCCCCC[N+]([O-])(CCCCCCCC(=O)OC(CCCCCCCC)CCCCCCCC)CCCNC(=O)CS(C)=O] |
| I 257 | 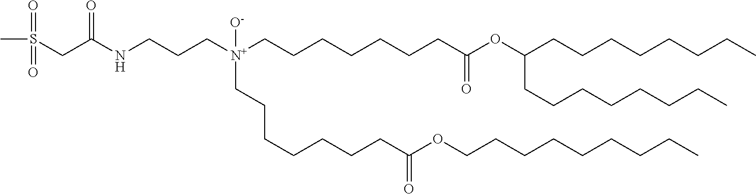 [SMILES: CCCCCCCCCOC(=O)CCCCCCC[N+]([O-])(CCCCCCCC(=O)OC(CCCCCCCC)CCCCCCCC)CCCNC(=O)CS(C)(=O)=O] |
| I 258 | 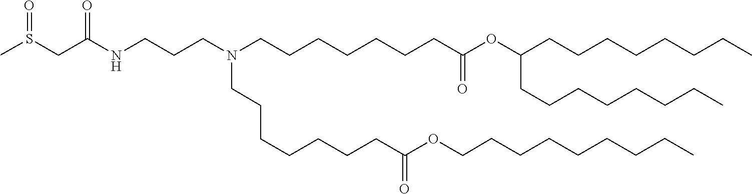 [SMILES: CCCCCCCCCOC(=O)CCCCCCCN(CCCCCCCC(=O)OC(CCCCCCCC)CCCCCCCC)CCCNC(=O)CS(C)=O] |
| I 259 | 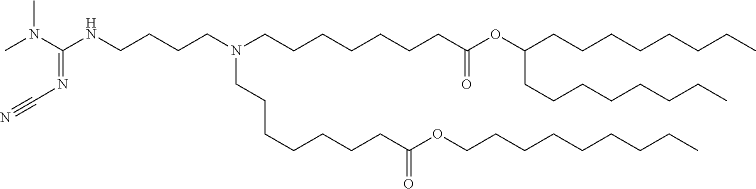 [SMILES: CCCCCCCCCOC(=O)CCCCCCCN(CCCCCCCC(=O)OC(CCCCCCCC)CCCCCCCC)CCCCN/C(=N/C#N)N(C)C] |
| I 260 | 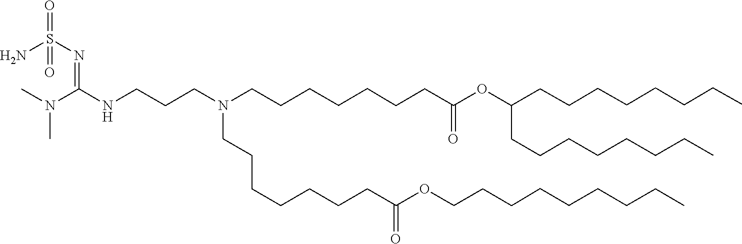 [SMILES: CCCCCCCCCOC(=O)CCCCCCCN(CCCCCCCC(=O)OC(CCCCCCCC)CCCCCCCC)CCCN/C(=N/S(N)(=O)=O)N(C)C] |
| I 261 | 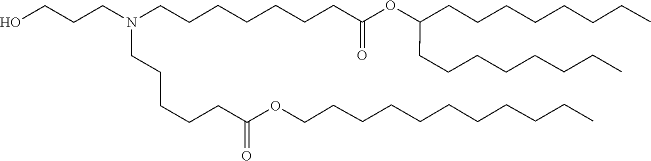 [SMILES: CCCCCCCCCCCOC(=O)CCCCCN(CCCO)CCCCCCCC(=O)OC(CCCCCCCC)CCCCCCCC] |
| I 262 | 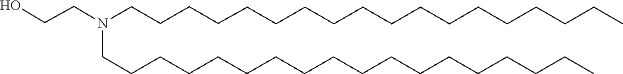 [SMILES: CCCCCCCCCCCCCCCCCCN(CCO)CCCCCCCCCCCCCCCCCC] |
| I 263 | 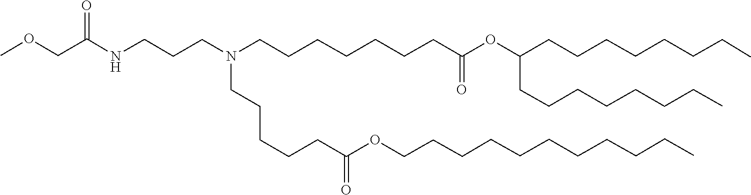 [SMILES: CCCCCCCCCCCOC(=O)CCCCCN(CCCCCCCC(=O)OC(CCCCCCCC)CCCCCCCC)CCCNC(=O)COC] |
| I 264 | 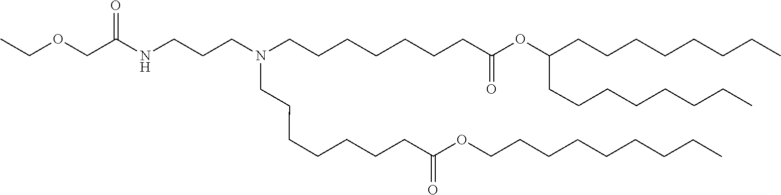 [SMILES: CCCCCCCCCOC(=O)CCCCCCCN(CCCCCCCC(=O)OC(CCCCCCCC)CCCCCCCC)CCCNC(=O)COCC] |
| I 265 | 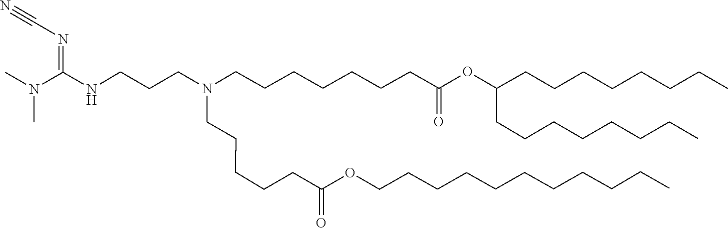 [SMILES: CCCCCCCCCCCOC(=O)CCCCCN(CCCCCCCC(=O)OC(CCCCCCCC)CCCCCCCC)CCCN/C(=N/C#N)N(C)C] |
| I 266 | 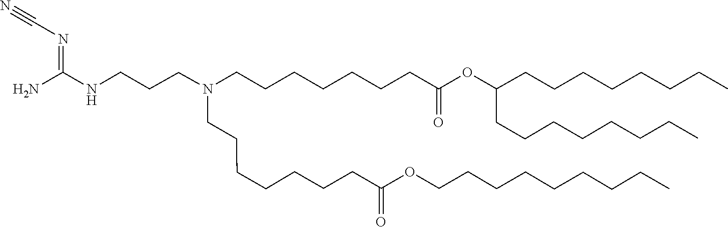 [SMILES: CCCCCCCCCOC(=O)CCCCCCCN(CCCCCCCC(=O)OC(CCCCCCCC)CCCCCCCC)CCCN/C(N)=N/C#N] |
| I 267 | 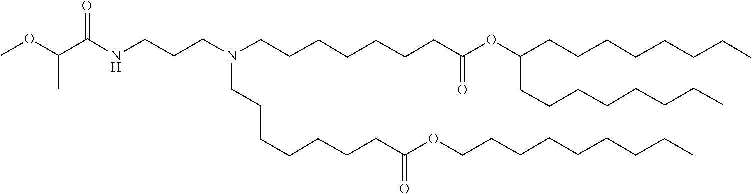 [SMILES: CCCCCCCCCOC(=O)CCCCCCCN(CCCCCCCC(=O)OC(CCCCCCCC)CCCCCCCC)CCCNC(=O)C(C)OC] |
| I 268 | 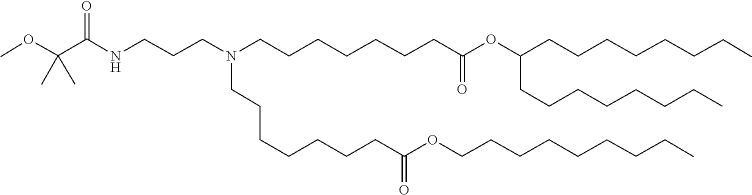 [SMILES: CCCCCCCCCOC(=O)CCCCCCCN(CCCCCCCC(=O)OC(CCCCCCCC)CCCCCCCC)CCCNC(=O)C(C)(C)OC] |
| I 269 | 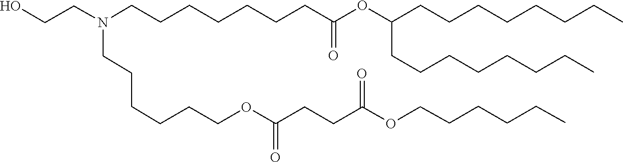 [SMILES: CCCCCCCCC(CCCCCCCC)OC(=O)CCCCCCCN(CCO)CCCCCCOC(=O)CCC(=O)OCCCCCC] |
| I 270 | 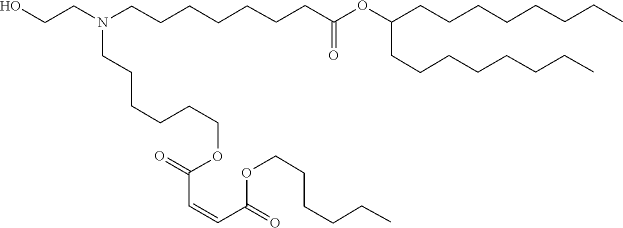 [SMILES: CCCCCCCCC(CCCCCCCC)OC(=O)CCCCCCCN(CCO)CCCCCCOC(=O)/C=C\C(=O)OCCCCCC] |
| I 271 | 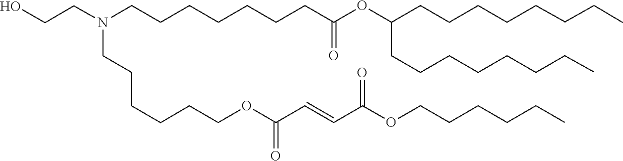 [SMILES: CCCCCCCCC(CCCCCCCC)OC(=O)CCCCCCCN(CCO)CCCCCCOC(=O)/C=C/C(=O)OCCCCCC] |
| I 272 | 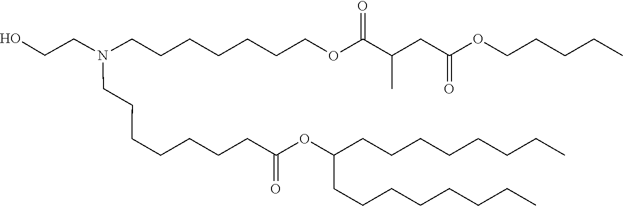 [SMILES: CCCCCCCCC(CCCCCCCC)OC(=O)CCCCCCCN(CCO)CCCCCCCOC(=O)C(C)CC(=O)OCCCCC] |
| I 273 | 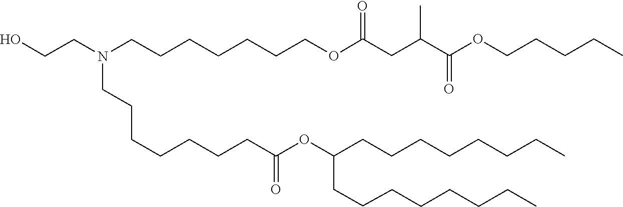 [SMILES: CCCCCCCCC(CCCCCCCC)OC(=O)CCCCCCCN(CCO)CCCCCCCOC(=O)CC(C)C(=O)OCCCCC] |
| I 274 | 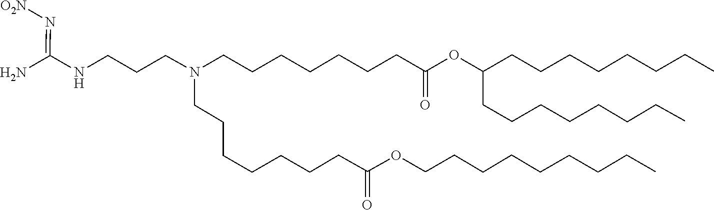 [SMILES: CCCCCCCCCOC(=O)CCCCCCCN(CCCCCCCC(=O)OC(CCCCCCCC)CCCCCCCC)CCCN/C(N)=N/[N+](=O)[O-]] |
| I 275 | 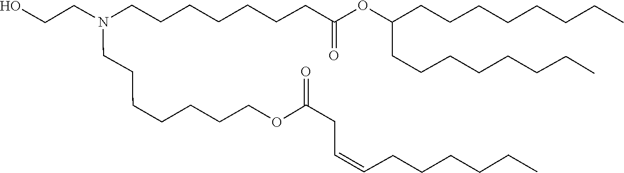 [SMILES: CCCCCC/C=C\CC(=O)OCCCCCCCN(CCO)CCCCCCCC(=O)OC(CCCCCCCC)CCCCCCCC] |
| I 276 | 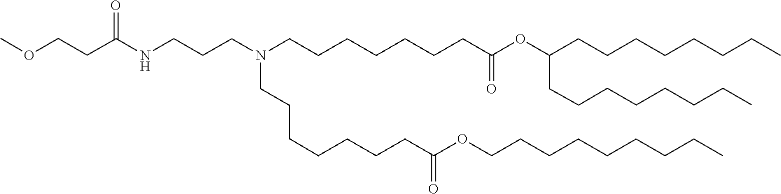 [SMILES: CCCCCCCCCOC(=O)CCCCCCCN(CCCCCCCC(=O)OC(CCCCCCCC)CCCCCCCC)CCCNC(=O)CCOC] |
| I 277 | 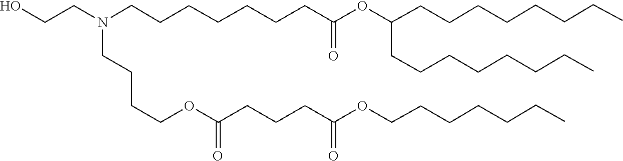 [SMILES: CCCCCCCCC(CCCCCCCC)OC(=O)CCCCCCCN(CCO)CCCCOC(=O)CCCC(=O)OCCCCCCC] |
| I 278 | 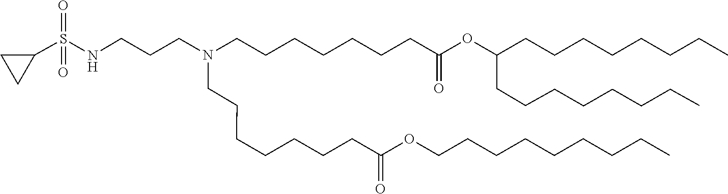 [SMILES: CCCCCCCCCOC(=O)CCCCCCCN(CCCCCCCC(=O)OC(CCCCCCCC)CCCCCCCC)CCCNS(=O)(=O)C1CC1] |
| I 279 | 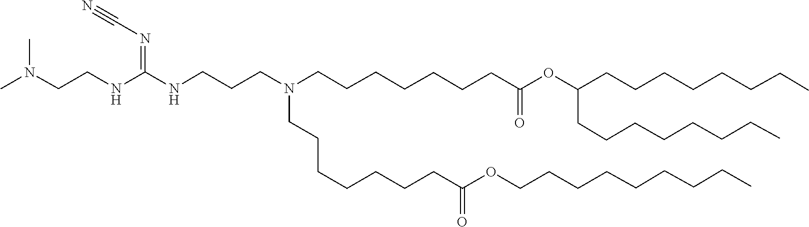 [SMILES: CCCCCCCCCOC(=O)CCCCCCCN(CCCCCCCC(=O)OC(CCCCCCCC)CCCCCCCC)CCCN/C(=N/C#N)NCCN(C)C] |
| I 280 | 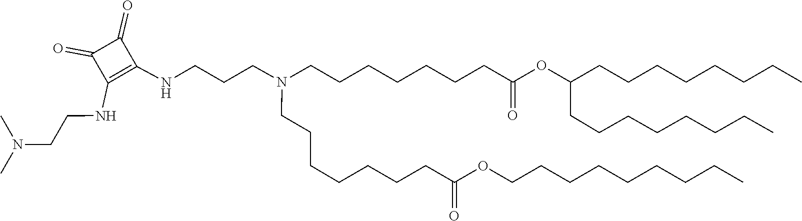 [SMILES: CCCCCCCCCOC(=O)CCCCCCCN(CCCCCCCC(=O)OC(CCCCCCCC)CCCCCCCC)CCCNc1c(NCCN(C)C)c(=O)c1=O] |
| I 281 | 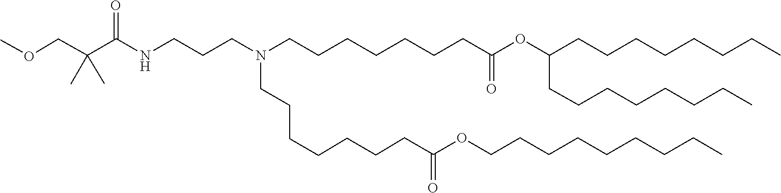 [SMILES: CCCCCCCCCOC(=O)CCCCCCCN(CCCCCCCC(=O)OC(CCCCCCCC)CCCCCCCC)CCCNC(=O)C(C)(C)COC] |
| I 282 | 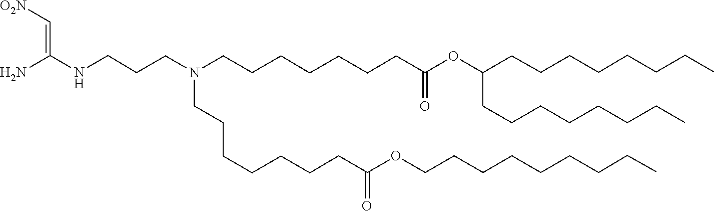 [SMILES: CCCCCCCCCOC(=O)CCCCCCCN(CCCCCCCC(=O)OC(CCCCCCCC)CCCCCCCC)CCCN/C(N)=C/[N+](=O)[O-]] |
| I 283 | 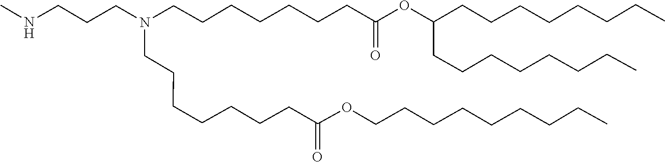 [SMILES: CCCCCCCCCOC(=O)CCCCCCCN(CCCCCCCC(=O)OC(CCCCCCCC)CCCCCCCC)CCCNC] |
| I 284 | 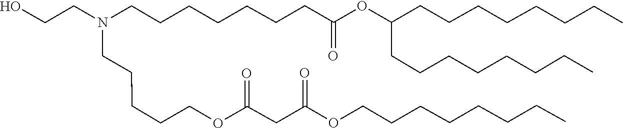 [SMILES: CCCCCCCCOC(=O)CC(=O)OCCCCCN(CCO)CCCCCCCC(=O)OC(CCCCCCCC)CCCCCCCC] |
| I 285 | 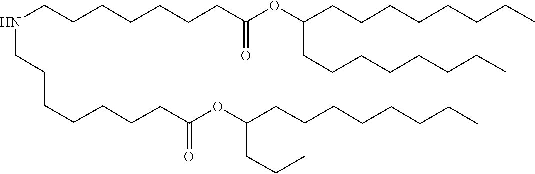 [SMILES: CCCCCCCCC(CCC)OC(=O)CCCCCCCNCCCCCCCC(=O)OC(CCCCCCCC)CCCCCCCC] |
| I 286 | 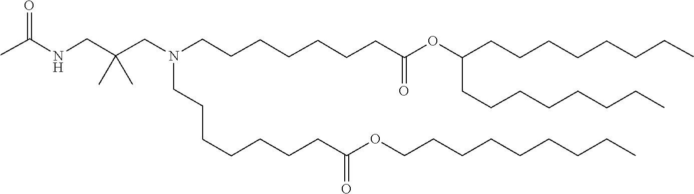 [SMILES: CCCCCCCCCOC(=O)CCCCCCCN(CCCCCCCC(=O)OC(CCCCCCCC)CCCCCCCC)CC(C)(C)CNC(C)=O] |
| I 287 | 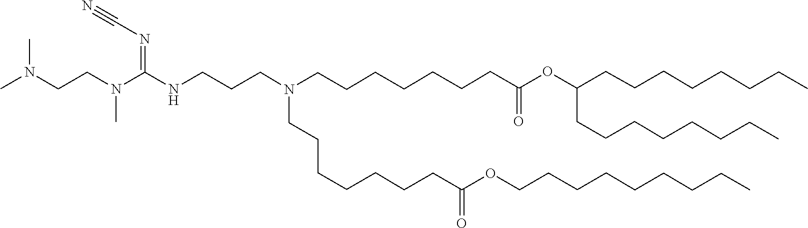 [SMILES: CCCCCCCCCOC(=O)CCCCCCCN(CCCCCCCC(=O)OC(CCCCCCCC)CCCCCCCC)CCCN/C(=N/C#N)N(C)CCN(C)C] |
| I 288 | 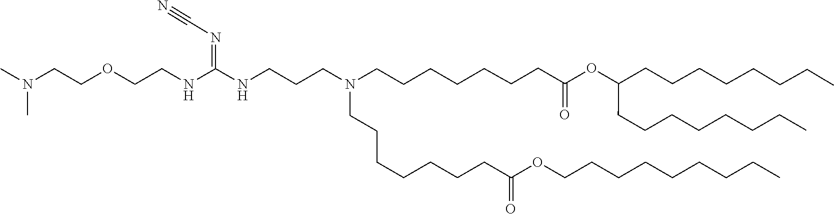 [SMILES: CCCCCCCCCOC(=O)CCCCCCCN(CCCCCCCC(=O)OC(CCCCCCCC)CCCCCCCC)CCCN/C(=N/C#N)NCCOCCN(C)C] |
| I 289 | 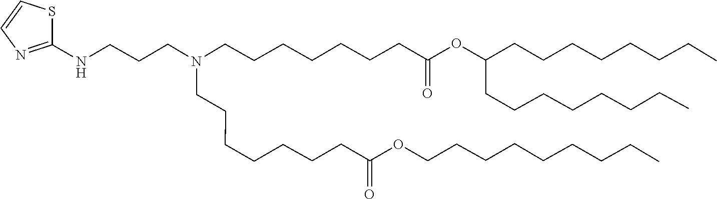 [SMILES: CCCCCCCCCOC(=O)CCCCCCCN(CCCCCCCC(=O)OC(CCCCCCCC)CCCCCCCC)CCCNc1nccs1] |
| I 290 | 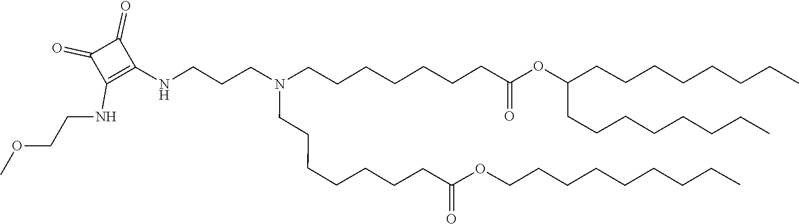 [SMILES: CCCCCCCCCOC(=O)CCCCCCCN(CCCCCCCC(=O)OC(CCCCCCCC)CCCCCCCC)CCCNc1c(NCCOC)c(=O)c1=O] |
| I 291 | 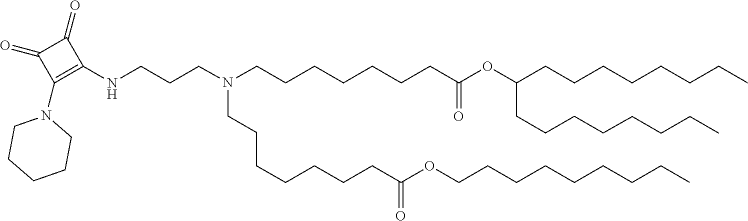 [SMILES: CCCCCCCCCOC(=O)CCCCCCCN(CCCCCCCC(=O)OC(CCCCCCCC)CCCCCCCC)CCCNc1c(N2CCCCC2)c(=O)c1=O] |
| I 292 | 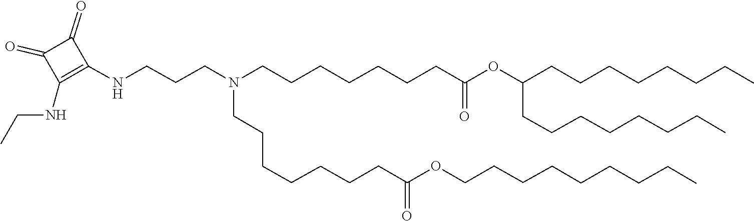 [SMILES: CCCCCCCCCOC(=O)CCCCCCCN(CCCCCCCC(=O)OC(CCCCCCCC)CCCCCCCC)CCCNc1c(NCC)c(=O)c1=O] |
| I 293 | 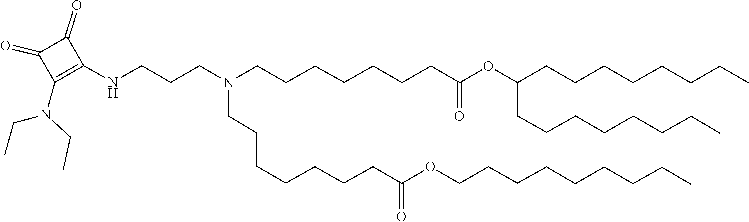 [SMILES: CCCCCCCCCOC(=O)CCCCCCCN(CCCCCCCC(=O)OC(CCCCCCCC)CCCCCCCC)CCCNc1c(N(CC)CC)c(=O)c1=O] |
| I 294 | 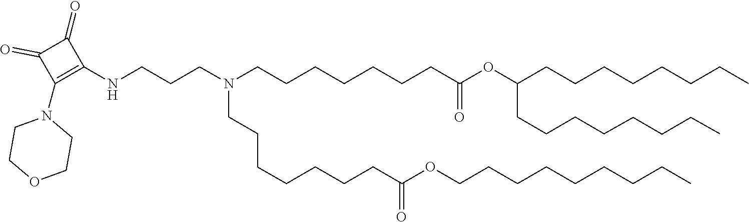 [SMILES: CCCCCCCCCOC(=O)CCCCCCCN(CCCCCCCC(=O)OC(CCCCCCCC)CCCCCCCC)CCCNc1c(N2CCOCC2)c(=O)c1=O] |
| I 295 | 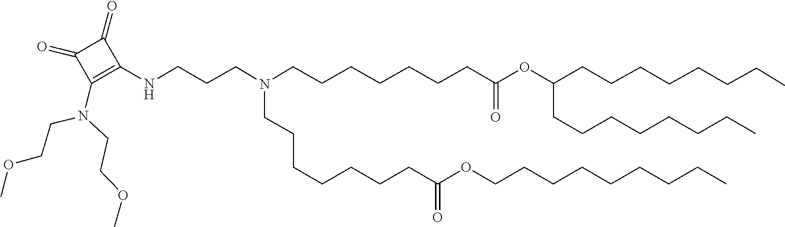 [SMILES: CCCCCCCCCOC(=O)CCCCCCCN(CCCCCCCC(=O)OC(CCCCCCCC)CCCCCCCC)CCCNc1c(N(CCOC)CCOC)c(=O)c1=O] |
| I 296 | 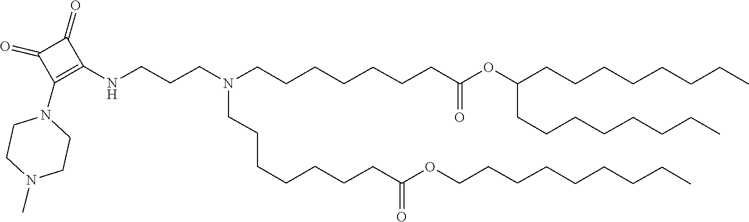 [SMILES: CCCCCCCCCOC(=O)CCCCCCCN(CCCCCCCC(=O)OC(CCCCCCCC)CCCCCCCC)CCCNc1c(N2CCN(C)CC2)c(=O)c1=O] |
| I 297 | 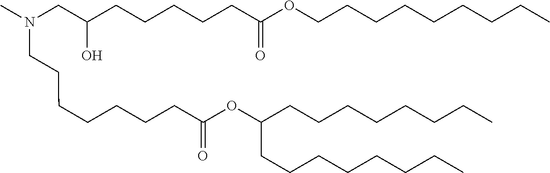 [SMILES: CCCCCCCCCOC(=O)CCCCCC(O)CN(C)CCCCCCCC(=O)OC(CCCCCCCC)CCCCCCCC] |
| I 298 | 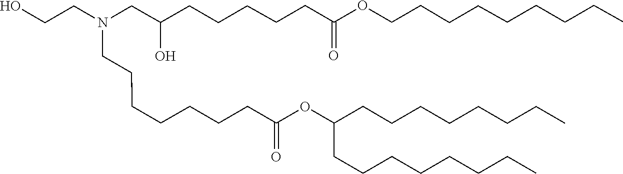 [SMILES: CCCCCCCCCOC(=O)CCCCCC(O)CN(CCO)CCCCCCCC(=O)OC(CCCCCCCC)CCCCCCCC] |
| I 299 | 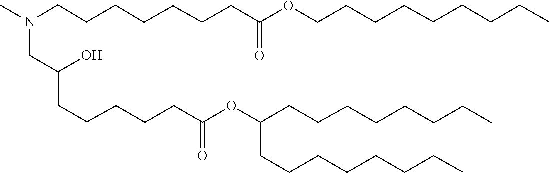 [SMILES: CCCCCCCCCOC(=O)CCCCCCCN(C)CC(O)CCCCCC(=O)OC(CCCCCCCC)CCCCCCCC] |
| I 300 | 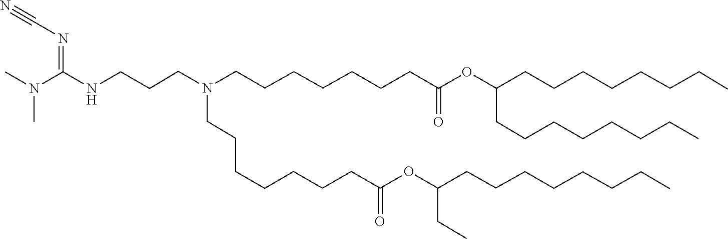 [SMILES: CCCCCCCCC(CC)OC(=O)CCCCCCCN(CCCCCCCC(=O)OC(CCCCCCCC)CCCCCCCC)CCCN/C(=N/C#N)N(C)C] |
| I 301 | 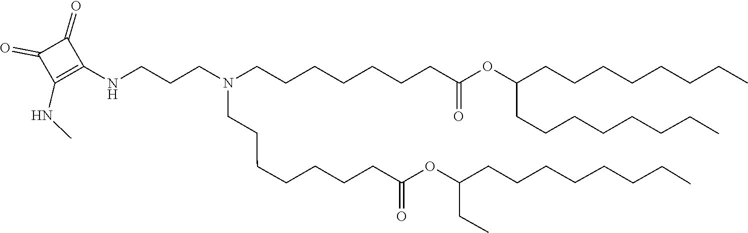 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| I 302 | 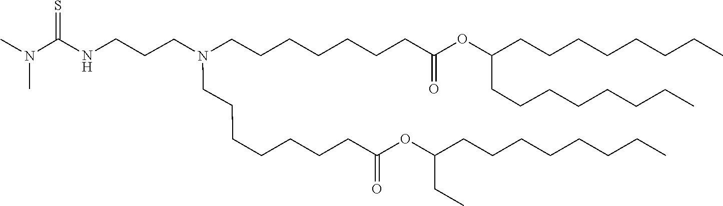 [SMILES: CCCCCCCCC(CC)OC(=O)CCCCCCCN(CCCCCCCC(=O)OC(CCCCCCCC)CCCCCCCC)CCCNC(=S)N(C)C] |
| I 303 | 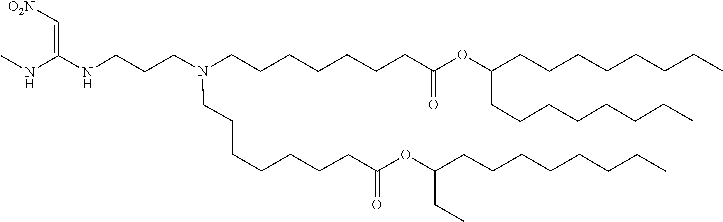 [SMILES: CCCCCCCCC(CC)OC(=O)CCCCCCCN(CCCCCCCC(=O)OC(CCCCCCCC)CCCCCCCC)CCCN/C(=C/[N+](=O)[O-])NC] |
| I 304 | 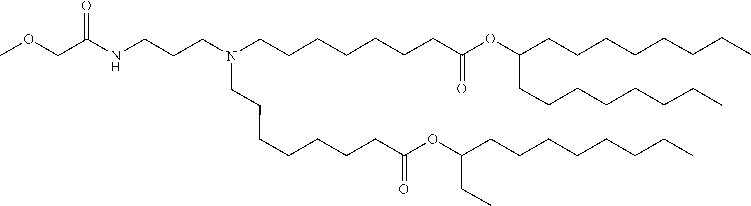 [SMILES: CCCCCCCCC(CC)OC(=O)CCCCCCCN(CCCCCCCC(=O)OC(CCCCCCCC)CCCCCCCC)CCCNC(=O)COC] |
| I 305 | 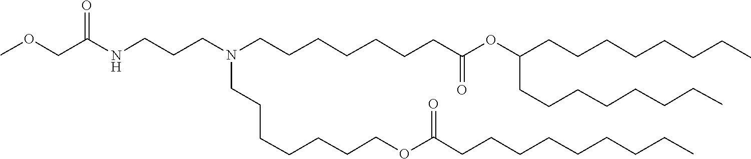 [SMILES: CCCCCCCCCC(=O)OCCCCCCCN(CCCCCCCC(=O)OC(CCCCCCCC)CCCCCCCC)CCCNC(=O)COC] |
| I 306 | 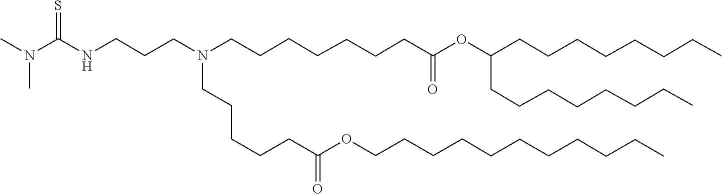 [SMILES: CCCCCCCCCCCOC(=O)CCCCCN(CCCCCCCC(=O)OC(CCCCCCCC)CCCCCCCC)CCCNC(=S)N(C)C] |
| I 307 | 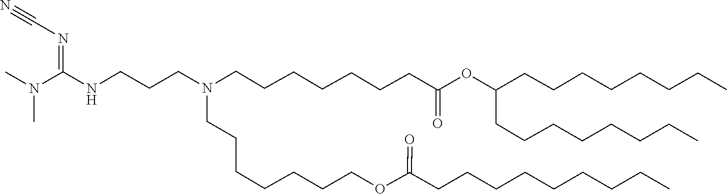 [SMILES: CCCCCCCCCC(=O)OCCCCCCCN(CCCCCCCC(=O)OC(CCCCCCCC)CCCCCCCC)CCCN/C(=N/C#N)N(C)C] |
| I 308 | 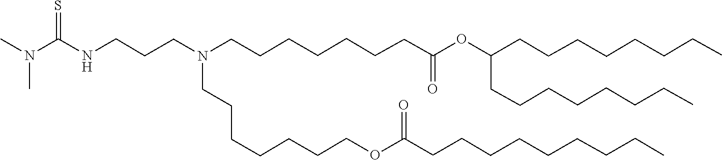 [SMILES: CCCCCCCCCC(=O)OCCCCCCCN(CCCCCCCC(=O)OC(CCCCCCCC)CCCCCCCC)CCCNC(=S)N(C)C] |
| I 309 | 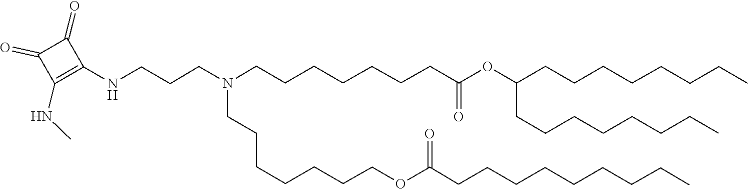 [SMILES: CCCCCCCCCC(=O)OCCCCCCCN(CCCCCCCC(=O)OC(CCCCCCCC)CCCCCCCC)CCCNc1c(NC)c(=O)c1=O] |
| I 310 | 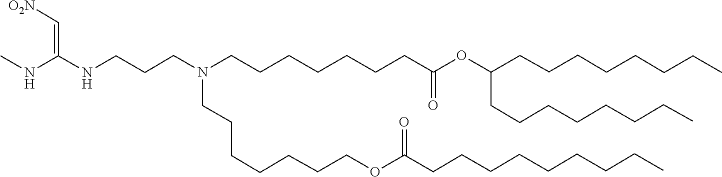 [SMILES: CCCCCCCCCC(=O)OCCCCCCCN(CCCCCCCC(=O)OC(CCCCCCCC)CCCCCCCC)CCCN/C(=C/[N+](=O)[O-])NC] |
| I 311 | 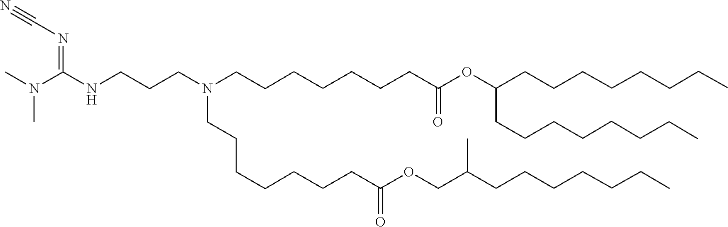 [SMILES: CCCCCCCCC(CCCCCCCC)OC(=O)CCCCCCCN(CCCCCCCC(=O)OCC(C)CCCCCCC)CCCN/C(=N/C#N)N(C)C] |
| I 312 | 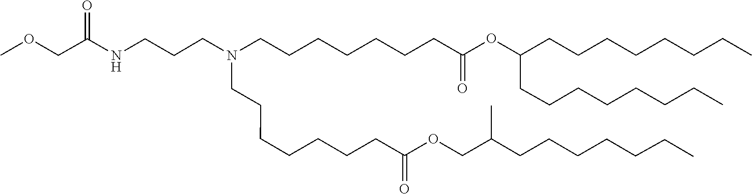 [SMILES: CCCCCCCCC(CCCCCCCC)OC(=O)CCCCCCCN(CCCCCCCC(=O)OCC(C)CCCCCCC)CCCNC(=O)COC] |
| I 313 | 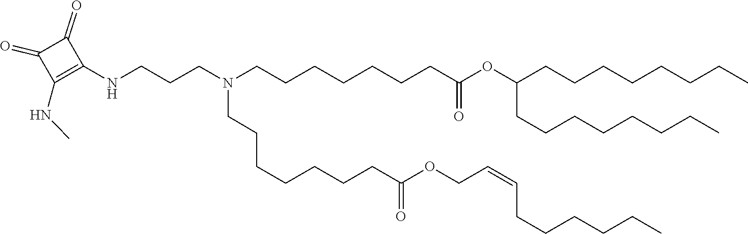 [SMILES: CCCCCC/C=C\COC(=O)CCCCCCCN(CCCCCCCC(=O)OC(CCCCCCCC)CCCCCCCC)CCCNc1c(NC)c(=O)c1=O] |
| I 314 | 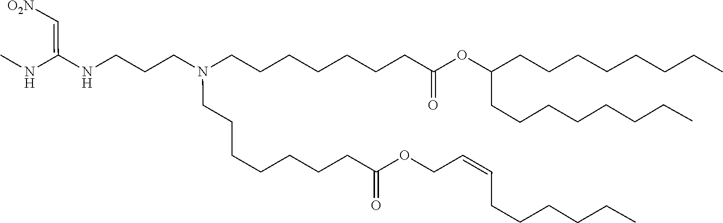 [SMILES: CCCCCC/C=C\COC(=O)CCCCCCCN(CCCCCCCC(=O)OC(CCCCCCCC)CCCCCCCC)CCCN/C(=C/[N+](=O)[O-])NC] |
| I 315 | 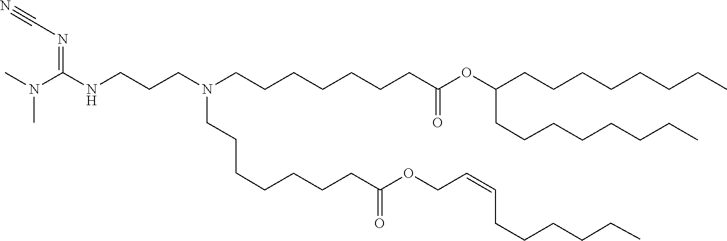 [SMILES: CCCCCC/C=C\COC(=O)CCCCCCCN(CCCCCCCC(=O)OC(CCCCCCCC)CCCCCCCC)CCCN/C(=N/C#N)N(C)C] |
| I 316 | 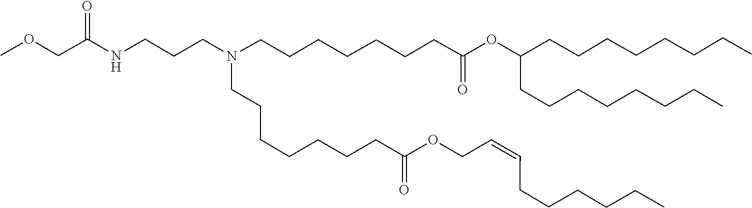 [SMILES: CCCCCC/C=C\COC(=O)CCCCCCCN(CCCCCCCC(=O)OC(CCCCCCCC)CCCCCCCC)CCCNC(=O)COC] |
| I 317 | 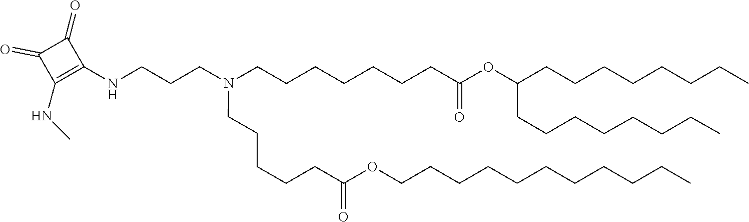 [SMILES: CCCCCCCCCCCOC(=O)CCCCCN(CCCCCCCC(=O)OC(CCCCCCCC)CCCCCCCC)CCCNc1c(NC)c(=O)c1=O] |
| I 318 | 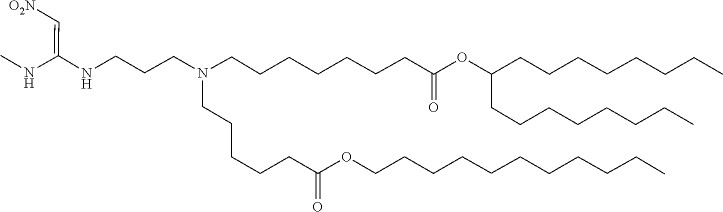 [SMILES: CCCCCCCCCCCOC(=O)CCCCCN(CCCCCCCC(=O)OC(CCCCCCCC)CCCCCCCC)CCCN/C(=C/[N+](=O)[O-])NC] |
| I 319 | 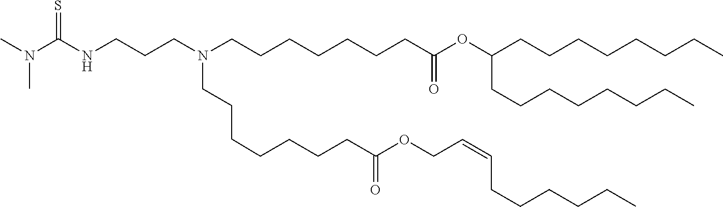 [SMILES: CCCCCC/C=C\COC(=O)CCCCCCCN(CCCCCCCC(=O)OC(CCCCCCCC)CCCCCCCC)CCCNC(=S)N(C)C] |
| I 320 | 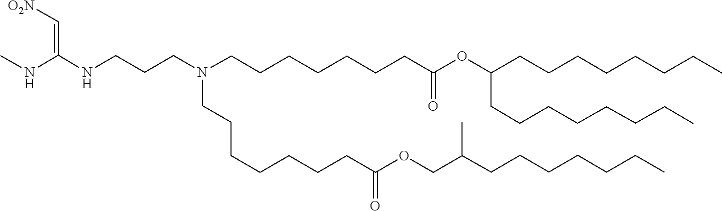 [SMILES: CCCCCCCCC(CCCCCCCC)OC(=O)CCCCCCCN(CCCCCCCC(=O)OCC(C)CCCCCCC)CCCN/C(=C/[N+](=O)[O-])NC] |
| I 321 | 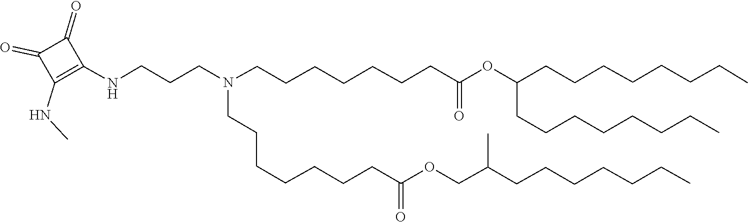 [SMILES: CCCCCCCCC(CCCCCCCC)OC(=O)CCCCCCCN(CCCCCCCC(=O)OCC(C)CCCCCCC)CCCNc1c(NC)c(=O)c1=O] |
| I 322 | 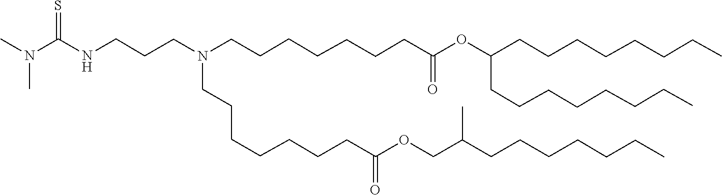 [SMILES: CCCCCCCCC(CCCCCCCC)OC(=O)CCCCCCCN(CCCCCCCC(=O)OCC(C)CCCCCCC)CCCNC(=S)N(C)C] |
| I 323 | 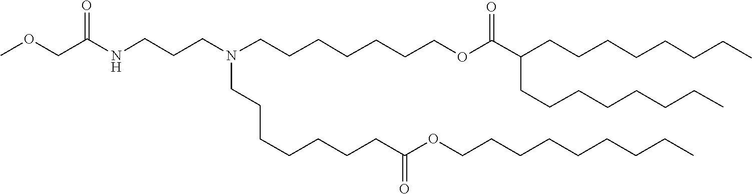 [SMILES: CCCCCCCCCOC(=O)CCCCCCCN(CCCCCCCOC(=O)C(CCCCCCCC)CCCCCCCC)CCCNC(=O)COC] |
| I 324 | 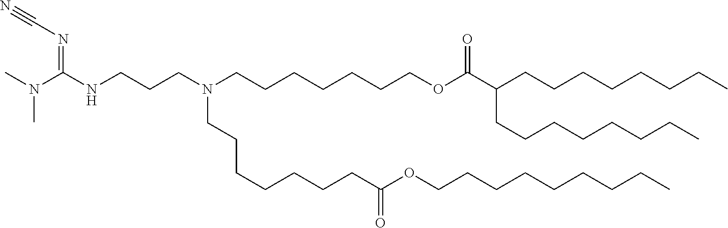 [SMILES: CCCCCCCCCOC(=O)CCCCCCCN(CCCCCCCOC(=O)C(CCCCCCCC)CCCCCCCC)CCCN/C(=N/C#N)N(C)C] |
| I 325 | 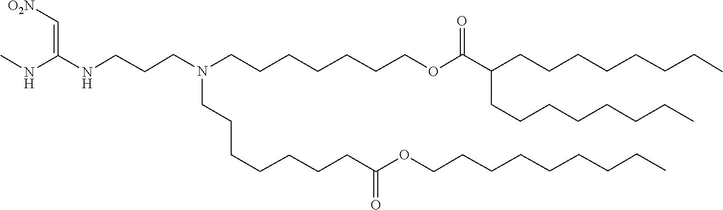 [SMILES: CCCCCCCCCOC(=O)CCCCCCCN(CCCCCCCOC(=O)C(CCCCCCCC)CCCCCCCC)CCCN/C(=C/[N+](=O)[O-])NC] |
| I 326 | 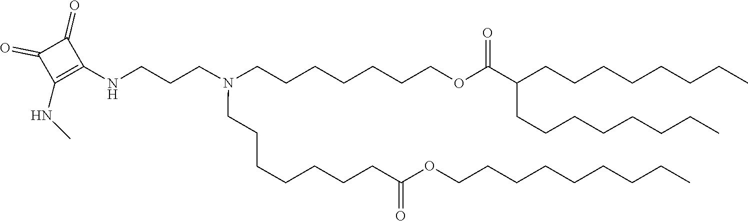 [SMILES: CCCCCCCCCOC(=O)CCCCCCCN(CCCCCCCOC(=O)C(CCCCCCCC)CCCCCCCC)CCCNc1c(NC)c(=O)c1=O] |
| I 327 | 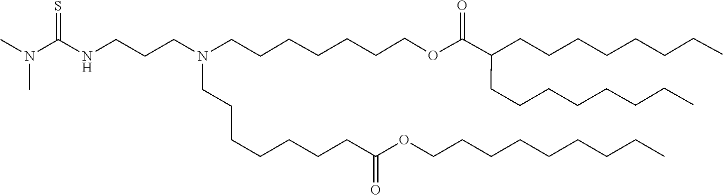 [SMILES: CCCCCCCCCOC(=O)CCCCCCCN(CCCCCCCOC(=O)C(CCCCCCCC)CCCCCCCC)CCCNC(=S)N(C)C] |
| I 328 | 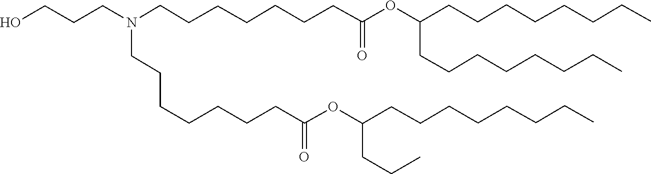 [SMILES: CCCCCCCCC(CCC)OC(=O)CCCCCCCN(CCCO)CCCCCCCC(=O)OC(CCCCCCCC)CCCCCCCC] |
| I 329 | 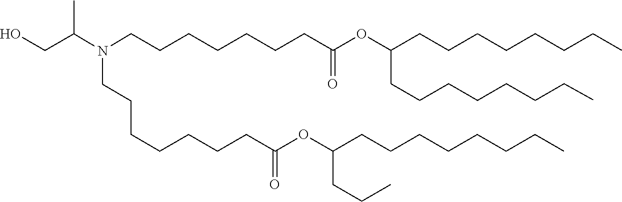 [SMILES: CCCCCCCCC(CCC)OC(=O)CCCCCCCN(CCCCCCCC(=O)OC(CCCCCCCC)CCCCCCCC)C(C)CO] |
| I 330 | 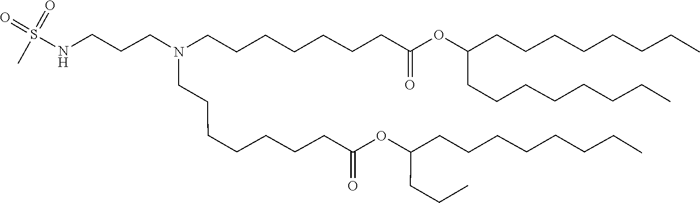 [SMILES: CCCCCCCCC(CCC)OC(=O)CCCCCCCN(CCCCCCCC(=O)OC(CCCCCCCC)CCCCCCCC)CCCNS(C)(=O)=O] |
| I 331 | 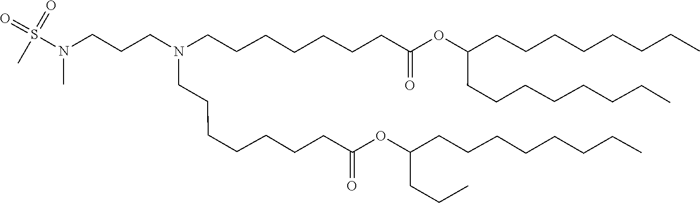 [SMILES: CCCCCCCCC(CCC)OC(=O)CCCCCCCN(CCCCCCCC(=O)OC(CCCCCCCC)CCCCCCCC)CCCN(C)S(C)(=O)=O] |
| I 332 | 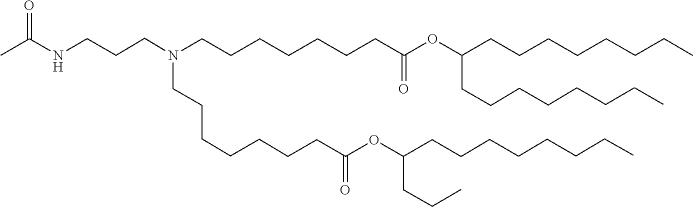 [SMILES: CCCCCCCCC(CCC)OC(=O)CCCCCCCN(CCCCCCCC(=O)OC(CCCCCCCC)CCCCCCCC)CCCNC(C)=O] |
| I 333 | 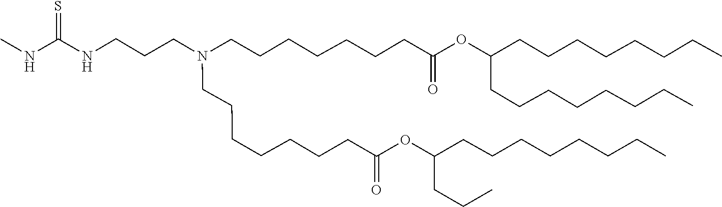 [SMILES: CCCCCCCCC(CCC)OC(=O)CCCCCCCN(CCCCCCCC(=O)OC(CCCCCCCC)CCCCCCCC)CCCNC(=S)NC] |
| I 334 | 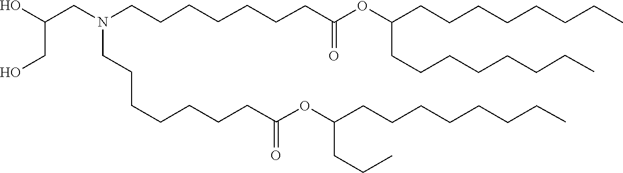 [SMILES: CCCCCCCCC(CCC)OC(=O)CCCCCCCN(CCCCCCCC(=O)OC(CCCCCCCC)CCCCCCCC)CC(O)CO] |
| I 335 | 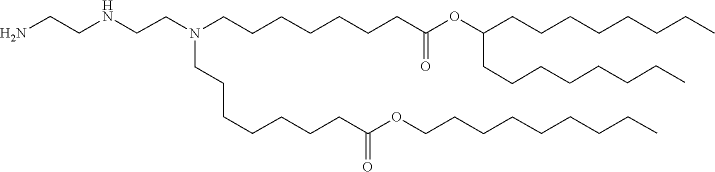 [SMILES: CCCCCCCCCOC(=O)CCCCCCCN(CCCCCCCC(=O)OC(CCCCCCCC)CCCCCCCC)CCNCCN] |
| I 336 | 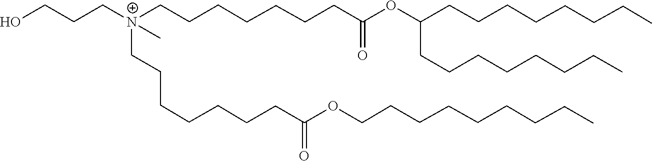 [SMILES: CCCCCCCCCOC(=O)CCCCCCC[N+](C)(CCCO)CCCCCCCC(=O)OC(CCCCCCCC)CCCCCCCC] |
| I 337 | 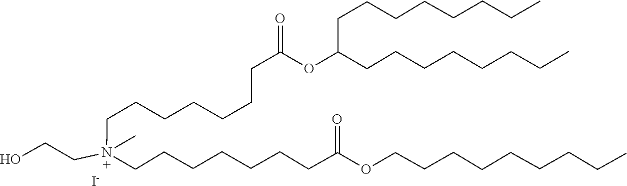 |
| I 338 | 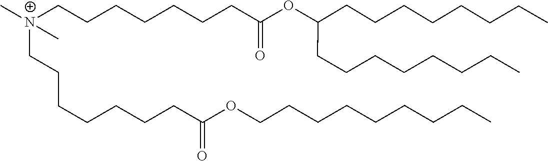 [SMILES: CCCCCCCCCOC(=O)CCCCCCC[N+](C)(C)CCCCCCCC(=O)OC(CCCCCCCC)CCCCCCCC] |
| I 339 | 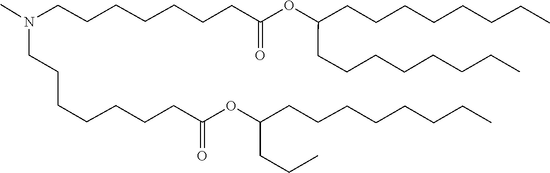 [SMILES: CCCCCCCCC(CCC)OC(=O)CCCCCCCN(C)CCCCCCCC(=O)OC(CCCCCCCC)CCCCCCCC] |
| I 340 | 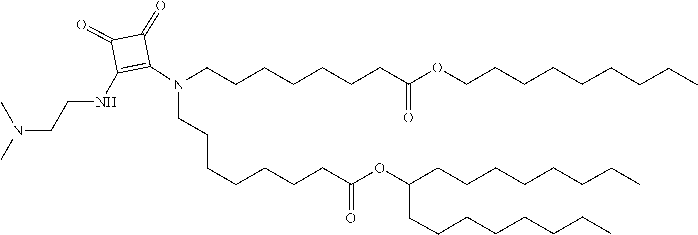 [SMILES: CCCCCCCCCOC(=O)CCCCCCCN(CCCCCCCC(=O)OC(CCCCCCCC)CCCCCCCC)c1c(NCCN(C)C)c(=O)c1=O] |
| I 341 | 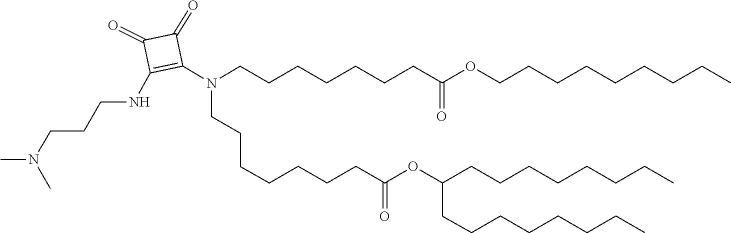 [SMILES: CCCCCCCCCOC(=O)CCCCCCCN(CCCCCCCC(=O)OC(CCCCCCCC)CCCCCCCC)c1c(NCCCN(C)C)c(=O)c1=O] |
| I 342 | 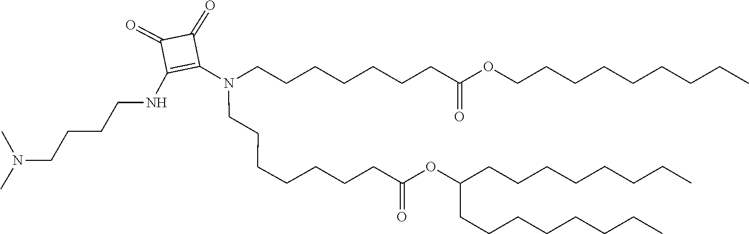 [SMILES: CCCCCCCCCOC(=O)CCCCCCCN(CCCCCCCC(=O)OC(CCCCCCCC)CCCCCCCC)c1c(NCCCCN(C)C)c(=O)c1=O] |
| I 343 | 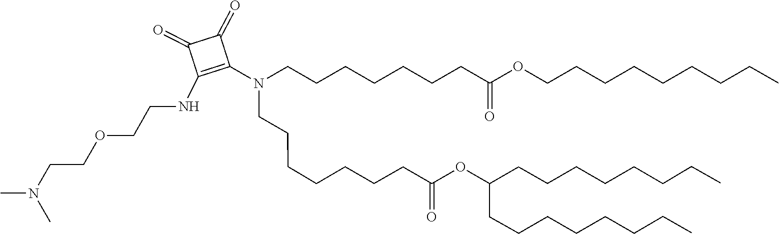 [SMILES: CCCCCCCCCOC(=O)CCCCCCCN(CCCCCCCC(=O)OC(CCCCCCCC)CCCCCCCC)c1c(NCCOCCN(C)C)c(=O)c1=O] |
| I 344 | 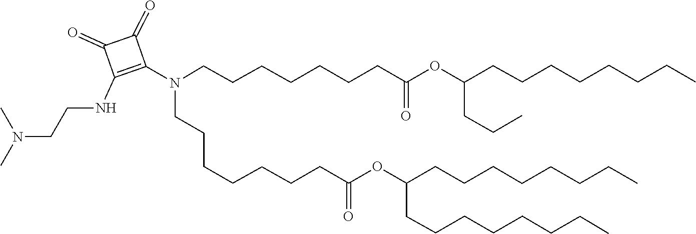 [SMILES: CCCCCCCCC(CCC)OC(=O)CCCCCCCN(CCCCCCCC(=O)OC(CCCCCCCC)CCCCCCCC)c1c(NCCN(C)C)c(=O)c1=O] |
| I 345 | 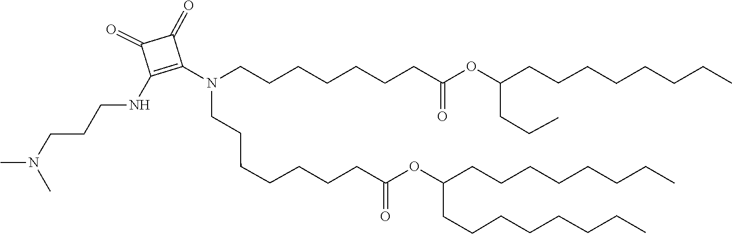 [SMILES: CCCCCCCCC(CCC)OC(=O)CCCCCCCN(CCCCCCCC(=O)OC(CCCCCCCC)CCCCCCCC)c1c(NCCCN(C)C)c(=O)c1=O] |
| I 346 | 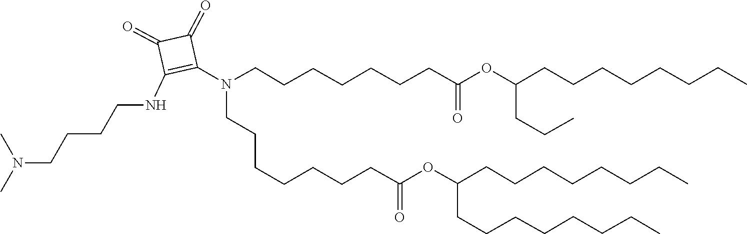 [SMILES: CCCCCCCCC(CCC)OC(=O)CCCCCCCN(CCCCCCCC(=O)OC(CCCCCCCC)CCCCCCCC)c1c(NCCCCN(C)C)c(=O)c1=O] |
| I 347 | 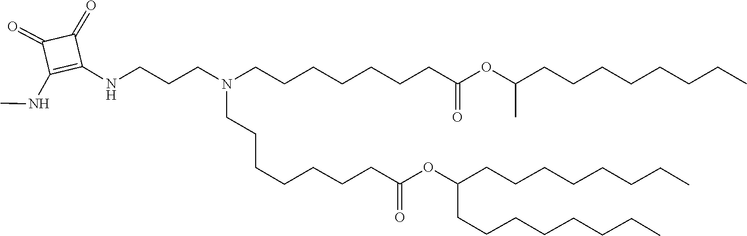 [SMILES: CCCCCCCCC(C)OC(=O)CCCCCCCN(CCCCCCCC(=O)OC(CCCCCCCC)CCCCCCCC)CCCNc1c(NC)c(=O)c1=O] |
| I 348 | 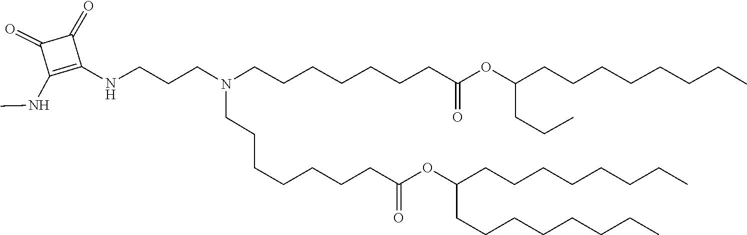 [SMILES: CCCCCCCCC(CCC)OC(=O)CCCCCCCN(CCCCCCCC(=O)OC(CCCCCCCC)CCCCCCCC)CCCNc1c(NC)c(=O)c1=O] |
| I 349 | 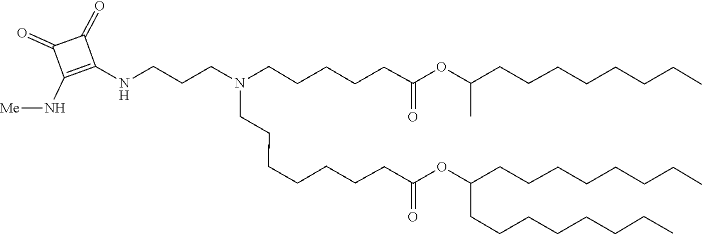 [SMILES: CCCCCCCCC(C)OC(=O)CCCCCN(CCCCCCCC(=O)OC(CCCCCCCC)CCCCCCCC)CCCNc1c(NC)c(=O)c1=O] |
| I 350 | 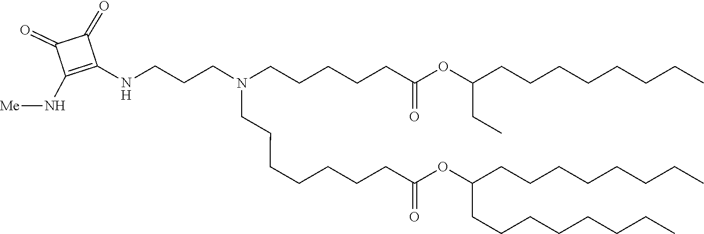 [SMILES: CCCCCCCCC(CC)OC(=O)CCCCCN(CCCCCCCC(=O)OC(CCCCCCCC)CCCCCCCC)CCCNc1c(NC)c(=O)c1=O] |
| I 351 | 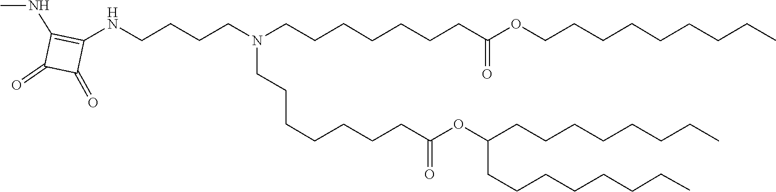 [SMILES: CCCCCCCCCOC(=O)CCCCCCCN(CCCCCCCC(=O)OC(CCCCCCCC)CCCCCCCC)CCCCNc1c(NC)c(=O)c1=O] |
| I 352 | 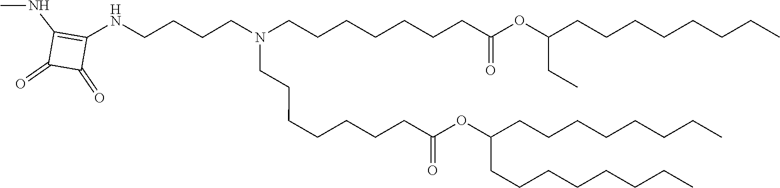 [SMILES: CCCCCCCCC(CC)OC(=O)CCCCCCCN(CCCCCCCC(=O)OC(CCCCCCCC)CCCCCCCC)CCCCNc1c(NC)c(=O)c1=O] |
| I 353 | 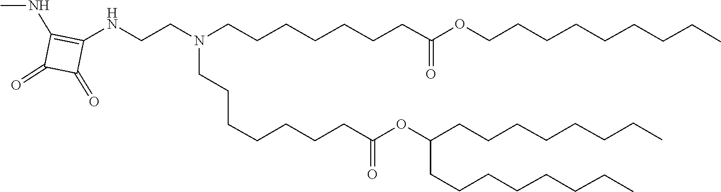 [SMILES: CCCCCCCCCOC(=O)CCCCCCCN(CCCCCCCC(=O)OC(CCCCCCCC)CCCCCCCC)CCNc1c(NC)c(=O)c1=O] |
| I 354 | 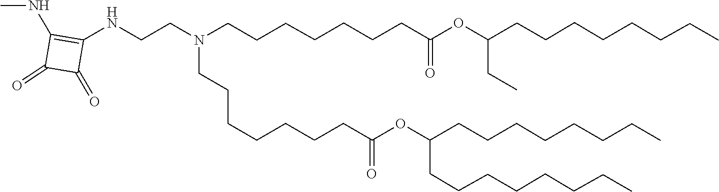 [SMILES: CCCCCCCCC(CC)OC(=O)CCCCCCCN(CCCCCCCC(=O)OC(CCCCCCCC)CCCCCCCC)CCNc1c(NC)c(=O)c1=O] |
| I 355 | 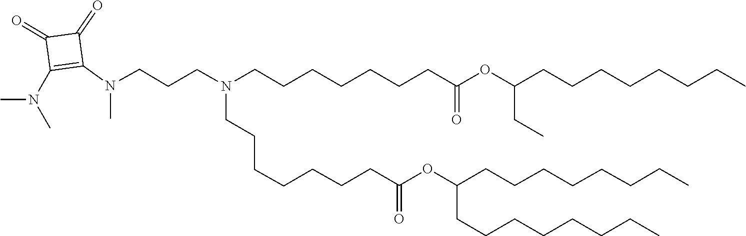 [SMILES: CCCCCCCCC(CC)OC(=O)CCCCCCCN(CCCCCCCC(=O)OC(CCCCCCCC)CCCCCCCC)CCCN(C)c1c(N(C)C)c(=O)c1=O] |

In some embodiments, a lipid of the disclosure comprises Compound I-340A:

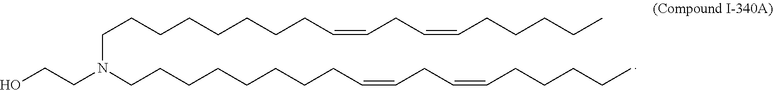 [SMILES: CCCCC/C=C\C/C=C\CCCCCCCCN(CCO)CCCCCCCC/C=C\C/C=C\CCCCC]

The central amine moiety of a lipid according to Formula (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), or (I VIIId) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some aspects, the ionizable lipids of the present disclosure may be one or more of compounds of formula I (I IX),

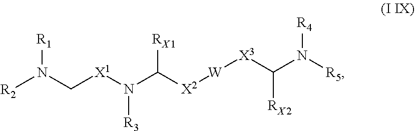

or salts or isomers thereof, wherein

W is

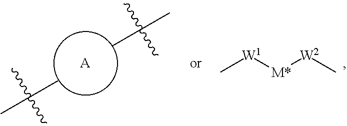

ring A is

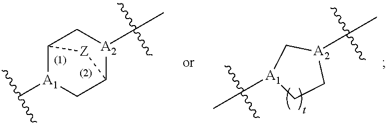

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl; X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

wherein when ring A is

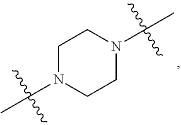 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (I IXa1)-(I IXa8):

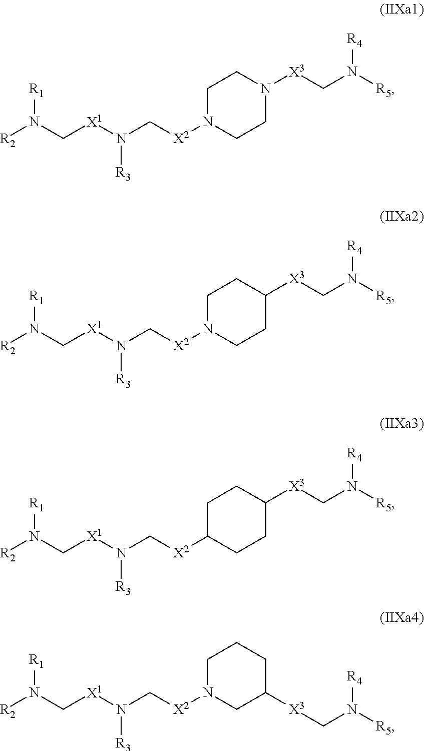

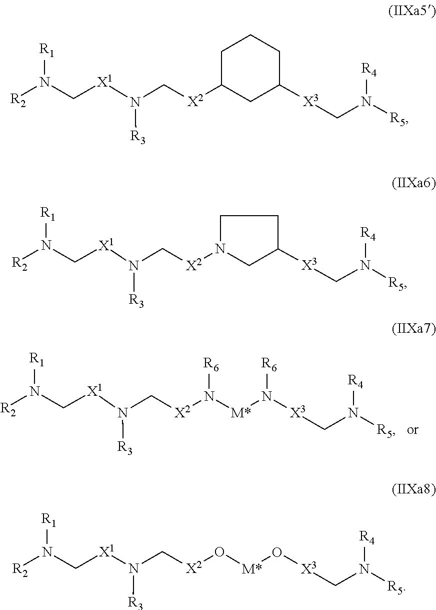

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

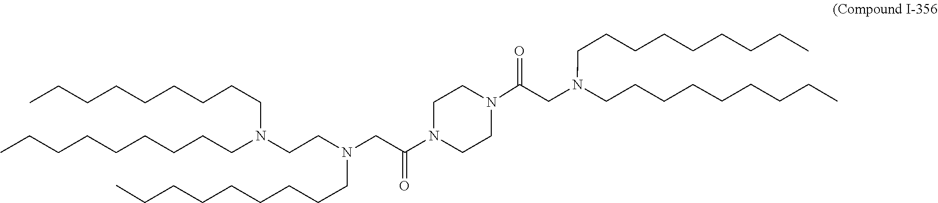 [SMILES: CCCCCCCCCN(CCCCCCCCC)CCN(CCCCCCCCC)CC(=O)N1CCN(C(=O)CN(CCCCCCCCC)CCCCCCCCC)CC1]

(also referred to herein as Compound M or I-M), or a salt thereof.
In some embodiments, the ionizable lipid is

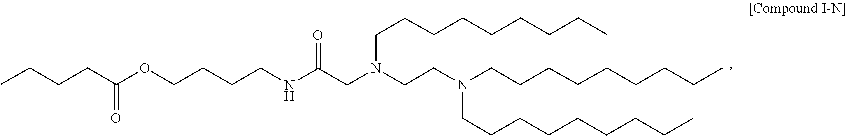 [SMILES: CCCCCCCCCN(CCCCCCCCC)CCN(CCCCCCCCC)CC(=O)NCCCCOC(=O)CCCC]

or a salt thereof.
In some embodiments, the ionizable lipid is

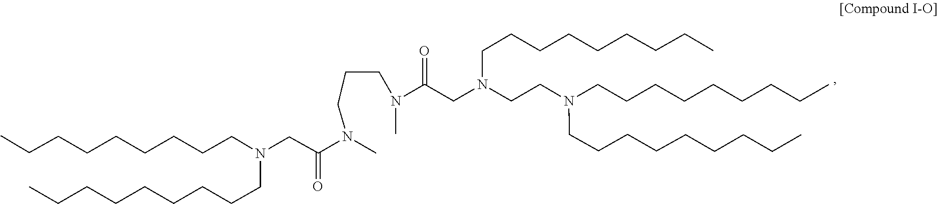 [SMILES: CCCCCCCCCN(CCCCCCCCC)CCN(CCCCCCCCC)CC(=O)N(C)CCCN(C)C(=O)CN(CCCCCCCCC)CCCCCCCCC]

or a salt thereof.
In some embodiments the ionizable lipid is

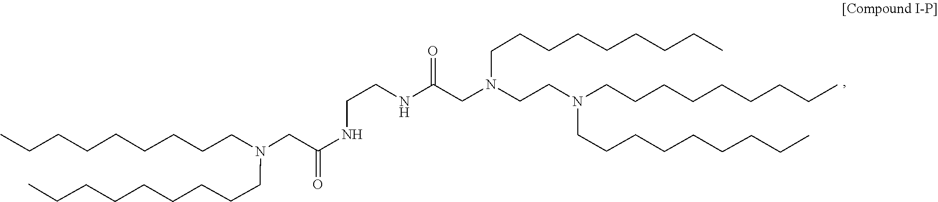 [SMILES: CCCCCCCCCN(CCCCCCCCC)CCN(CCCCCCCCC)CC(=O)NCCNC(=O)CN(CCCCCCCCC)CCCCCCCCC]

or a salt thereof.
In some embodiments, the ionizable lipid is

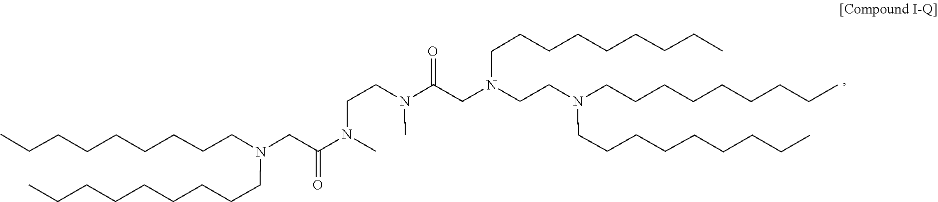 [SMILES: CCCCCCCCCN(CCCCCCCCC)CCN(CCCCCCCCC)CC(=O)N(C)CCN(C)C(=O)CN(CCCCCCCCC)CCCCCCCCC]

or a salt thereof.

The central amine moiety of a lipid according to any of the Formulae herein, e.g. a compound having any of Formula (I I), (I IA), (I IB), (I I), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some embodiments, the amount the ionizable amino lipid of the invention, e.g. a compound having any of Formula (I), (IA), (IB), (I I), (IIa), (IIb), (IIc), (IId), (IIe), (IIf), (IIg), (I II), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8)) (each of these preceded by the letter I for clarity) ranges from about 1 mol % to 99 mol % in the lipid composition.

In one embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition. In one embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 45 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 40 mol % in the lipid composition.

In one specific embodiment, the amount of the ionizable amino lipid of the invention, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity) is about 50 mol % in the lipid composition.

In addition to the ionizable amino lipid disclosed herein, e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8), (each of these preceded by the letter I for clarity) the lipid-based composition (e.g., lipid nanoparticle) disclosed herein can comprise additional components such as cholesterol and/or cholesterol analogs, non-cationic helper lipids, structural lipids, PEG-lipids, and any combination thereof.

Additional ionizable lipids of the invention can be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), (13Z,165Z)—N,N-dimethyl-3-nonydocosa-13-16-dien-1-amine (L608), 2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids of the invention can also be the compounds disclosed in International Publication No. WO 2017/075531 A1, hereby incorporated by reference in its entirety. For example, the ionizable amino lipids include, but not limited to:

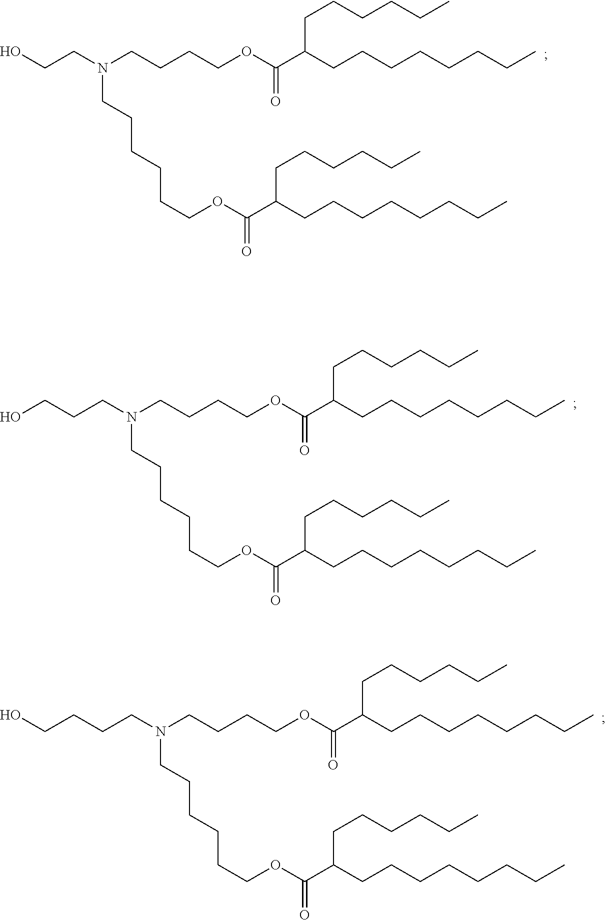 [SMILES: CCCCCCCCC(CCCCCC)C(=O)OCCCCCCN(CCO)CCCCOC(=O)C(CCCCCC)CCCCCCCC ; CCCCCCCCC(CCCCCC)C(=O)OCCCCCCN(CCCO)CCCCOC(=O)C(CCCCCC)CCCCCCCC ; CCCCCCCCC(CCCCCC)C(=O)OCCCCCCN(CCCCO)CCCCOC(=O)C(CCCCCC)CCCCCCCC]

and any combination thereof.

Ionizable lipids of the invention can also be the compounds disclosed in International Publication No. WO 2015/199952 A1, hereby incorporated by reference in its entirety. For example, the ionizable amino lipids include, but not limited to:

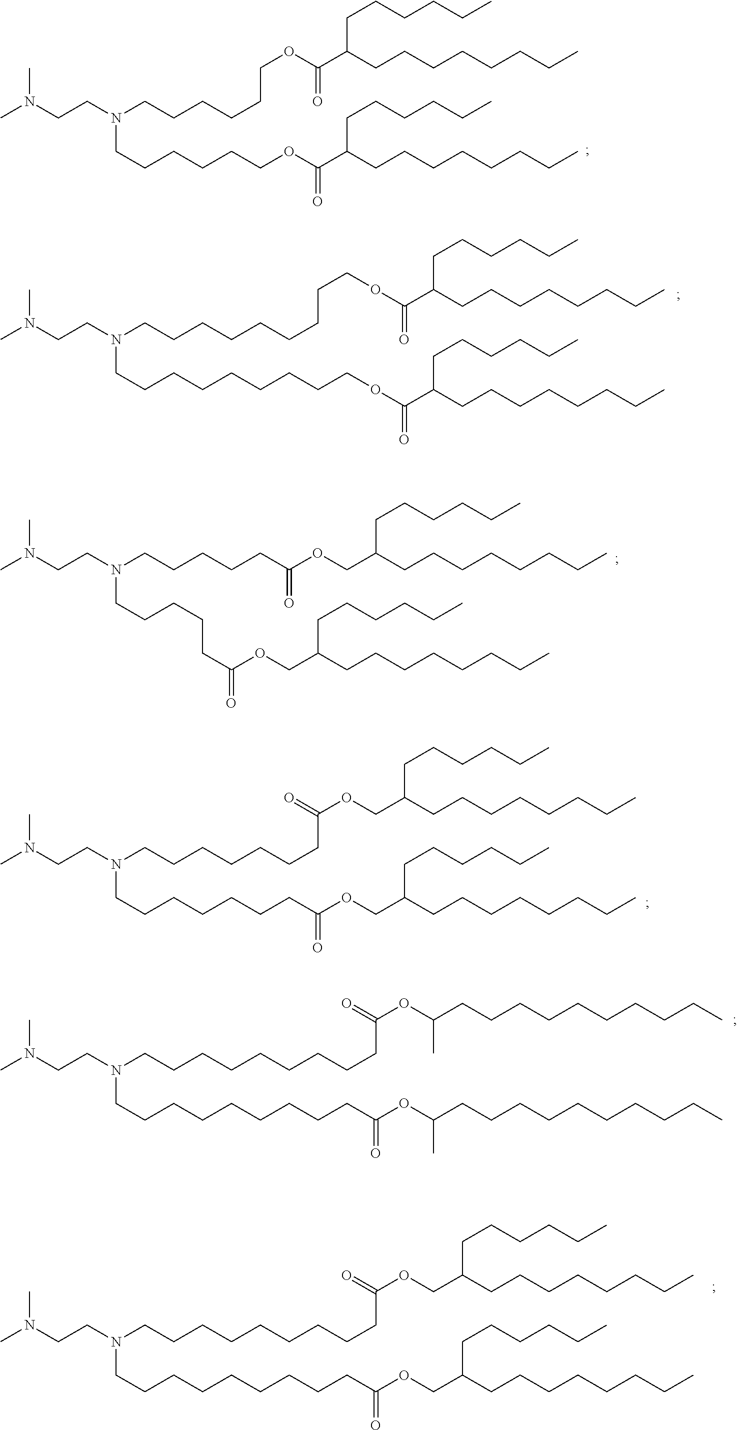

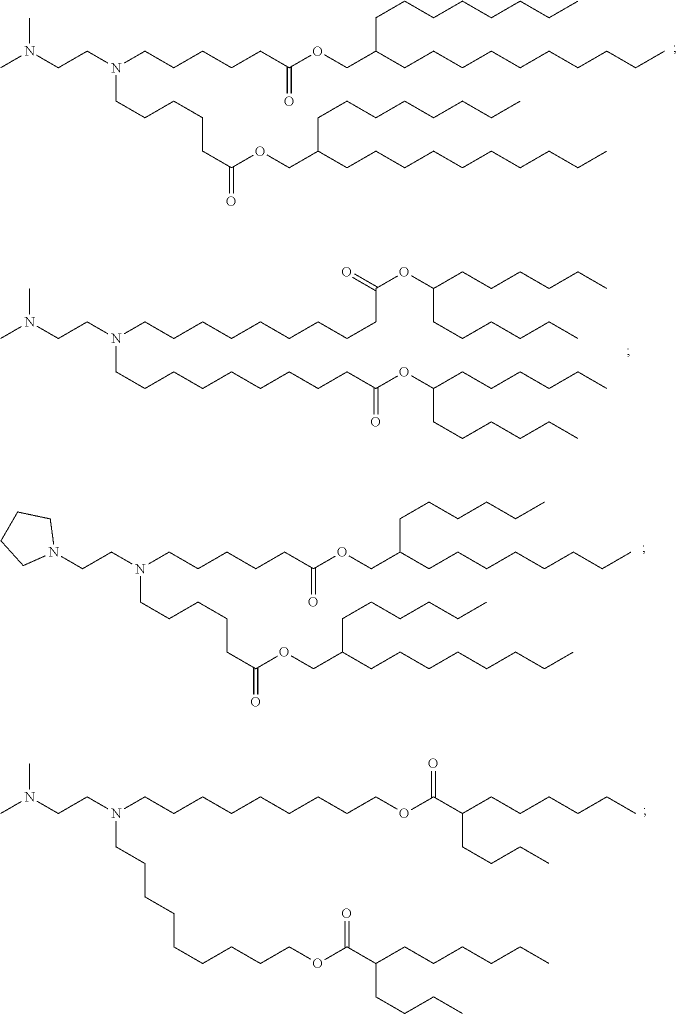

and any combination thereof.

In any of the foregoing or related aspects, the ionizable lipid of the LNP of the disclosure comprises a compound included in any e.g. a compound having any of Formula (I), (IA), (IB), (II), (IIa), (IIb), (IIc), (IId), (IIe), (IIf), (IIg), (III), (VI), (VI-a), (VII), (VIII), (VIIa), (VIIIa), (VIIIb), (VIIb-1), (VIIb-2), (VIIb-3), (VIIc), (VIId), (VIIIc), (VIIId), (IX), (IXa1), (IXa2), (IXa3), (IXa4), (IXa5), (IXa6), (IXa7), or (IXa8) (each of these preceded by the letter I for clarity).

In any of the foregoing or related aspects, the ionizable lipid of the LNP of the disclosure comprises a compound comprising any of Compound Nos. 11-356.

In any of the foregoing or related aspects, the ionizable lipid of the LNP of the disclosure comprises at least one compound selected from the group consisting of: Compound Nos. I 18 (also referred to as Compound X), I 25 (also referred to as Compound Y), I 48, I 50, I 109, I 111, 1113, I 181, I 182, I 244, I 292, I 301, I 321, I 322, I 326, I 328, I 330, I 331, and I 332. In another embodiment, the ionizable lipid of the LNP of the disclosure comprises a compound selected from the group consisting of: Compound Nos. I 18 (also referred to as Compound X), I 25 (also referred to as Compound Y), I 48, I 50, I 109, I 111, I 181, I 182, I 292, I 301, I 321, I 326, I 328, and I 330. In another embodiment, the ionizable lipid of the LNP of the disclosure comprises a compound selected from the group consisting of: Compound Nos. I 182, I 301, I 321, and I 326.

In any of the foregoing or related aspects, the synthesis of compounds of the invention, e.g. compounds comprising any of Compound Nos. 1-356, follows the synthetic descriptions in U.S. Provisional Patent Application No. 62/733,315, filed Sep. 19, 2018.

### Representative Synthetic Routes:

### Compound I-182: Heptadecan-9-yl 8-((3-((2-(methylamino)-3,4-dioxocyclobut-1-en-1-yl)amino)propyl)(8-(nonyloxy)-8-oxooctyl)amino)octanoate 3-Methoxy-4-(methylamino)cyclobut-3-ene-1,2-dione

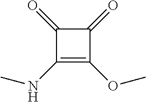 [0551] Chemical Formula: C₆H₇NO₃
Molecular Weight: 141.13

To a solution of 3,4-dimethoxy-3-cyclobutene-1,2-dione (1 g, 7 mmol) in 100 mL diethyl ether was added a 2M methylamine solution in THF (3.8 mL, 7.6 mmol) and a ppt. formed almost immediately. The mixture was stirred at rt for 24 hours, then filtered, the filter solids washed with diethyl ether and air-dried. The filter solids were dissolved in hot EtOAc, filtered, the filtrate allowed to cool to room temp., then cooled to 0° C. to give a ppt. This was isolated via filtration, washed with cold EtOAc, air-dried, then dried under vacuum to give 3-methoxy-4-(methylamino)cyclobut-3-ene-1,2-dione (0.70 g, 5 mmol, 73%) as a white solid. ¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### Heptadecan-9-yl 8-((3-((2-(methylamino)-3,4-dioxocyclobut-1-en-1-yl)amino)propyl)(8-(nonyloxy)-8-oxooctyl)amino)octanoate

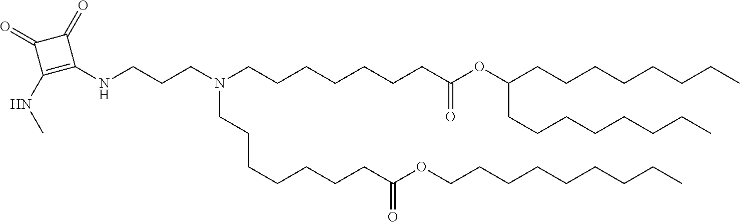 [SMILES: CCCCCCCCCOC(=O)CCCCCCCN(CCCCCCCC(=O)OC(CCCCCCCC)CCCCCCCC)CCCNc1c(NC)c(=O)c1=O] [0555] Chemical Formula: C₅₀H₉₃N3O₆
Molecular Weight: 832.31

To a solution of heptadecan-9-yl 8-((3-aminopropyl)(8-(nonyloxy)-8-oxooctyl)amino)octanoate (200 mg, 0.28 mmol) in 10 mL ethanol was added 3-methoxy-4-(methylamino)cyclobut-3-ene-1,2-dione (39 mg, 0.28 mmol) and the resulting colorless solution stirred at rt for 20 hours after which no starting amine remained by LC/MS. The solution was concentrated in vacuo and the residue purified by silica gel chromatography (0-100% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-(nonyloxy)-8-oxooctyl)amino)octanoate (138 mg, 0.17 mmol, 60%) as a gummy white solid. UPLC/ELSD: RT=3. min. MS (ES): m/z (MH⁺) 833.4 for C₅₁H₉₅N₃O₆. ¹H NMR (300 MHz, CDCl₃) δ: ppm 7.86 (br. s., 1H); 4.86 (quint., 1H, J=6 Hz); 4.05 (t, 2H, J=6 Hz); 3.92 (d, 2H, J=3 Hz); 3.20 (s, 6H); 2.63 (br. s, 2H); 2.42 (br. s, 3H); 2.28 (m, 4H); 1.74 (br. s, 2H); 1.61 (m, 8H); 1.50 (m, 5H); 1.41 (m, 3H); 1.25 (br. m, 47H); 0.88 (t, 9H, J=7.5 Hz).

### Compound I-301: Heptadecan-9-yl 8-((3-((2-(methylamino)-3,4-dioxocyclobut-1-en-1-yl)amino)propyl)(8-oxo-8-(undecan-3-yloxy)octyl)amino)octanoate

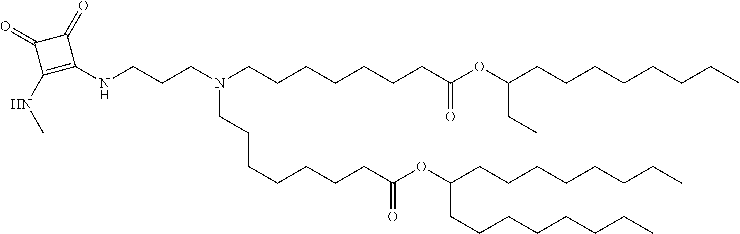 [SMILES: CCCCCCCCC(CC)OC(=O)CCCCCCCN(CCCCCCCC(=O)OC(CCCCCCCC)CCCCCCCC)CCCNc1c(NC)c(=O)c1=O] [0559] Chemical Formula: C₅₂H₉₇N₃O₆
Molecular Weight: 860.36

Compound I-301 was prepared analogously to compound 182 except that heptadecan-9-yl 8-((3-aminopropyl)(8-oxo-8-(undecan-3-yloxy)octyl)amino)octanoate (500 mg, 0.66 mmol) was used instead of heptadecan-9-yl 8-((3-aminopropyl)(8-(nonyloxy)-8-oxooctyl)amino)octanoate. Following an aqueous workup the residue was purified by silica gel chromatography (0-50% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-oxo-8-(undecan-3-yloxy)octyl)amino)octanoate (180 mg, 32%) as a white waxy solid. HPLC/UV (254 nm): RT=6.77 min. MS (CI): m/z (MH⁺) 860.7 for C₅₂H₉₇N₃O₆. ¹H NMR (300 MHz, CDCl₃): δ ppm 4.86-4.79 (m, 2H); 3.66 (bs, 2H); 3.25 (d, 3H, J=4.9 Hz); 2.56-2.52 (m, 2H); 2.42-2.37 (m, 4H); 2.28 (dd, 4H, J=2.7 Hz, 7.4 Hz); 1.78-1.68 (m, 3H); 1.64-1.50 (m, 16H); 1.48-1.38 (m, 6H); 1.32-1.18 (m, 43H); 0.88-0.84 (m, 12H).

(ii) Cholesterol/Structural Lipids

The immune cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

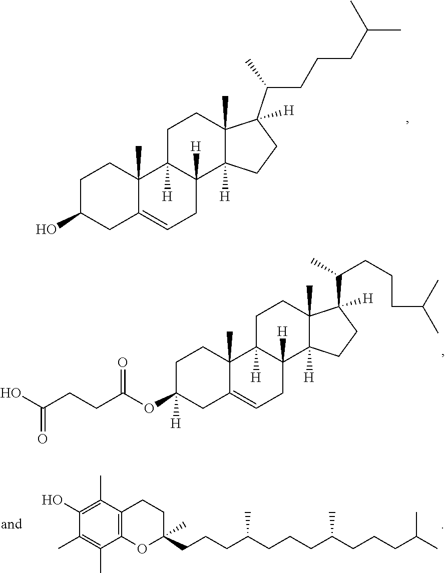

The immune cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. Structural lipids can include, but are not limited to, sterols (e.g., phytosterols or zoosterols).

In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds S1-148 in Tables 1-16 herein.

In certain embodiments, the structural lipid is cholesterol. In certain embodiments, the structural lipid is an analog of cholesterol.

In certain embodiments, the structural lipid is alpha-tocopherol.

In an aspect, the structural lipid of the invention features a compound having the structure of Formula SI:

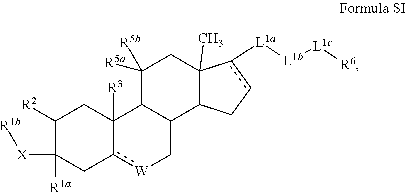

where

R^{1a} is H, optionally substituted C₁-C₆ alkyl, optionally substituted C₂-C₆ alkenyl, or optionally substituted C₂-C₆ alkynyl;

X is O or S;

R^{1b} is H, optionally substituted C₁-C₆ alkyl, or

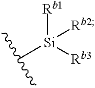

each of R^{b1}, R^{b2}, and R^{b3} is, independently, optionally substituted C₁-C₆ alkyl or optionally substituted C₆-C₁₀ aryl;

R² is H or OR^{A}, where R^{A} is H or optionally substituted C₁-C₆ alkyl;

R³ is H or

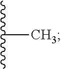

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

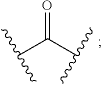 [SMILES: *C(*)=O]

L^{1a} is absent,

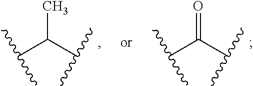

L^{1b} is absent,

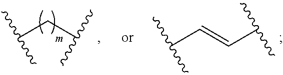

m is 1, 2, or 3;

L^{1c} is absent,

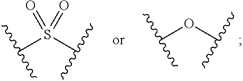

and

R⁶ is optionally substituted C₃-C₁₀ cycloalkyl, optionally substituted C₃-C₁₀ cycloalkenyl, optionally substituted C₆-C₁₀ aryl, optionally substituted C₂-C₉ heterocyclyl, or optionally substituted C₂-C₉ heteroaryl, or a pharmaceutically acceptable salt thereof.

In some embodiments, the compound has the structure of Formula SIa:

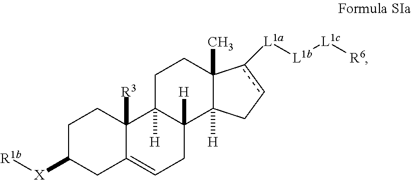

or a pharmaceutically acceptable salt thereof.

In some embodiments, the compound has the structure of Formula SIb:

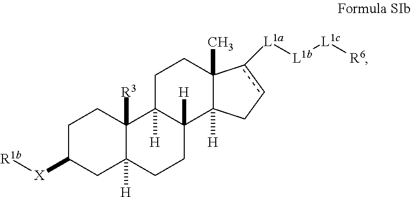

or a pharmaceutically acceptable salt thereof.

In some embodiments, the compound has the structure of Formula SIc:

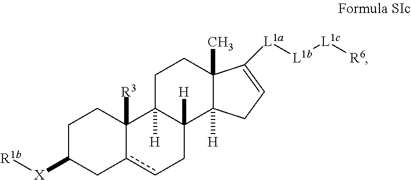

or a pharmaceutically acceptable salt thereof.

In some embodiments, the compound has the structure of Formula SId:

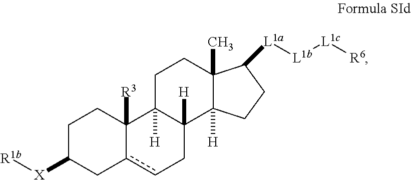

or a pharmaceutically acceptable salt thereof.

In some embodiments, L^{1a} is absent. In some embodiments, L^{1a} is

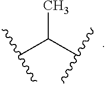 [SMILES: *C(*)C]

In some embodiments, L^{1a} is

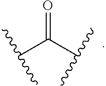 [SMILES: *C(*)=O]

In some embodiments, L^{1b} is absent. In some embodiments, L^{1b} is

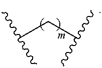

In some embodiments, L^{1b} is

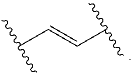 [SMILES: */C=C/*]

In some embodiments, m is 1 or 2. In some embodiments, m is 1. In some embodiments, m is 2.

In some embodiments, L^{1c} is absent. In some embodiments, L^{1c} is

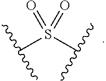 [SMILES: *S(*)(=O)=O]

In some embodiments, L^{1c} is.

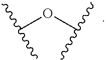 [SMILES: *O*]

In some embodiments, R⁶ is optionally substituted C₆-C₁₀ aryl.

In some embodiments, R⁶ is

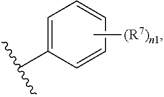

where

n1 is 0, 1, 2, 3, 4, or 5; and

each R⁷ is, independently, halo or optionally substituted C₁-C₆ alkyl.

In some embodiments, each R⁷ is, independently,

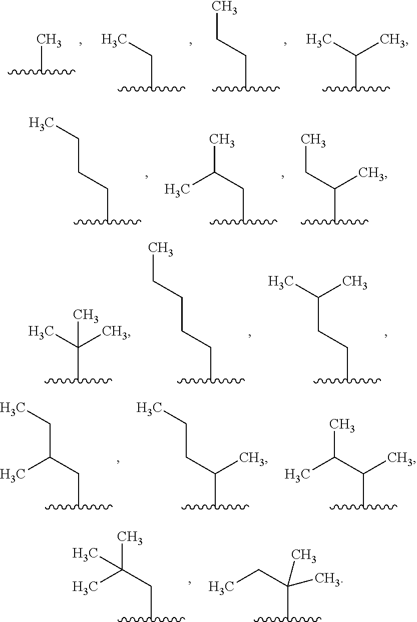

In some embodiments, n1 is 0, 1, or 2. In some embodiments, n is 0. In some embodiments, n1 is 1. In some embodiments, n1 is 2.

In some embodiments, R⁶ is optionally substituted C₃-C₁₀ cycloalkyl.

In some embodiments, R⁶ is optionally substituted C₃-C₁₀ monocycloalkyl.

In some embodiments, R⁶ is

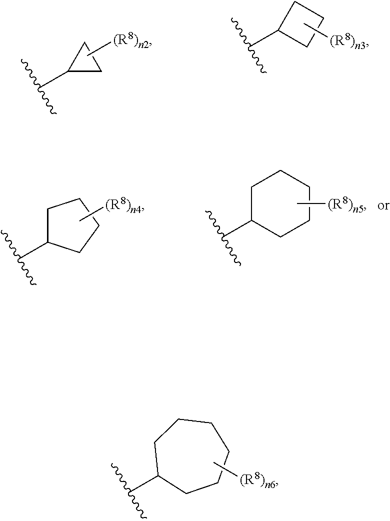

where

n2 is 0, 1, 2, 3, 4, or 5;

n3 is 0, 1, 2, 3, 4, 5, 6, or 7;

n4 is 0, 1, 2, 3, 4, 5, 6, 7, 8, or 9;

n5 is 0, 1, 2, 3, 4, 5, 6, 7, 8, 9, 10, or 11;

n6 is 0, 1, 2, 3, 4, 5, 6, 7, 8, 9, 10, 11, 12, or 13; and

each R⁸ is, independently, halo or optionally substituted C₁-C₆ alkyl.

In some embodiments, each R⁸ is, independently,

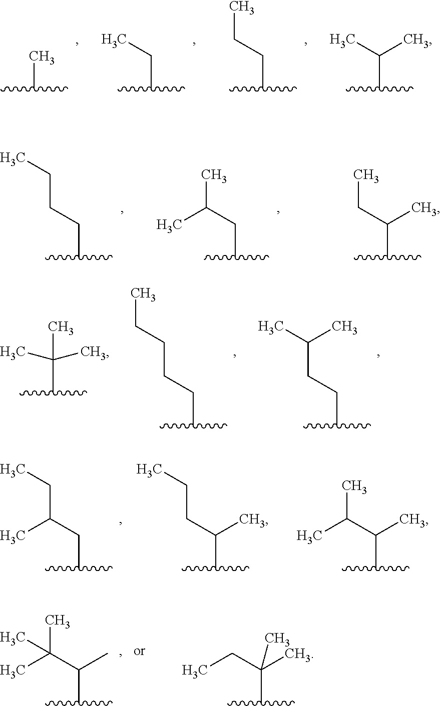

In some embodiments, R⁶ is optionally substituted C₃-C₁₀ polycycloalkyl.

In some embodiments, R⁶ is

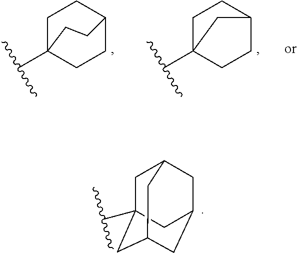 [SMILES: *C12CCC(CC1)CC2 ; *C12CCC(CC1)C2 ; *C12CC3CC(CC(C3)C1)C2]

In some embodiments, R⁶ is optionally substituted C₃-C₁₀ cycloalkenyl.

In some embodiments, R⁶ is

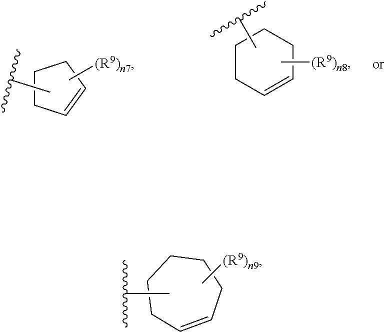

where

n7 is 0, 1, 2, 3, 4, 5, 6, or 7;

n8 is 0, 1, 2, 3, 4, 5, 6, 7, 8, or 9;

n9 is 0, 1, 2, 3, 4, 5, 6, 7, 8, 9, 10, or 11; and

each R⁹ is, independently, halo or optionally substituted C₁-C₆ alkyl.

In some embodiments, R⁶ is

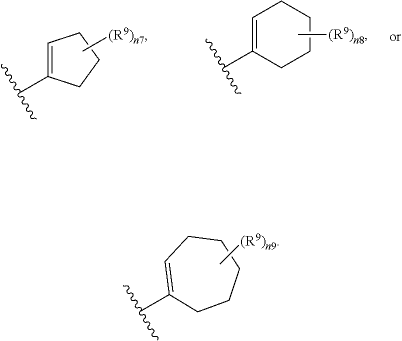

In some embodiments, each R is, independently,

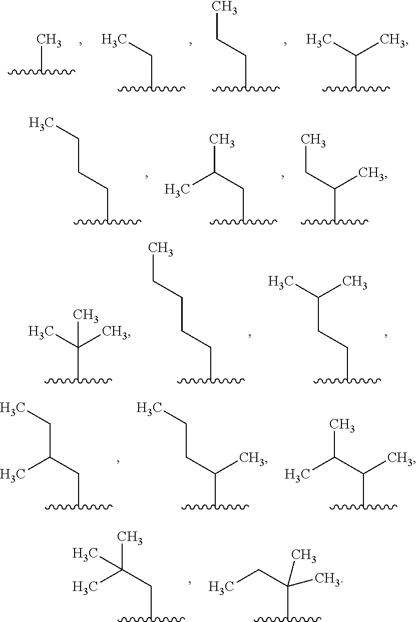

In some embodiments, R⁶ is optionally substituted C₂-C₉ heterocyclyl.

In some embodiments, R⁶ is

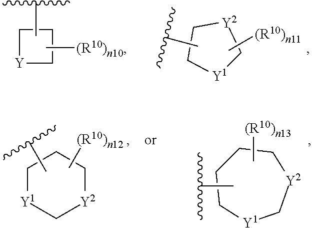

where

n10 is 0, 1, 2, 3, 4, or 5;

n11 is 0, 1, 2, 3, 4, or 5;

n12 is 0, 1, 2, 3, 4, 5, 6, or 7;

n13 is 0, 1, 2, 3, 4, 5, 6, 7, 8, or 9;

each R¹⁰ is, independently, halo or optionally substituted C₁-C₆ alkyl; and

each of Y¹ and Y² is, independently, O, S, NR^{B}, or CR^{11a}R^{11b},

where R^{B} is H or optionally substituted C₁-C₆ alkyl;

each of R^{11a} a and R^{11b} is, independently, H, halo, or optionally substituted C₁-C₆ alkyl; and

if Y² is CR^{11a}R^{11b}, then Y¹ is O, S, or NR^{B}.

In some embodiments, Y¹ is O.

In some embodiments, Y² is O. In some embodiments, Y² is CR^{11a}R^{11b}

In some embodiments, each R¹⁰ is, independently,

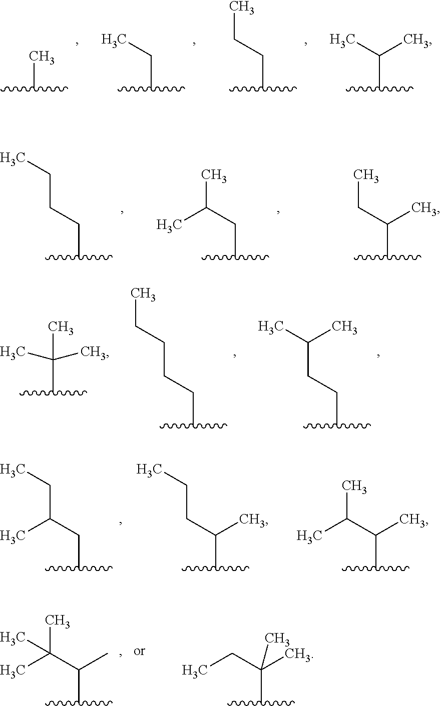

In some embodiments, R⁶ is optionally substituted C₂-C₉ heteroaryl.

In some embodiments, R⁶ is

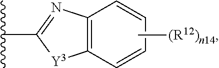

where

Y³ is NR^{C}, O, or S

n14 is 0, 1, 2, 3, or 4;

R^{C} is H or optionally substituted C₁-C₆ alkyl; and

each R¹² is, independently, halo or optionally substituted C₁-C₆ alkyl.

In some embodiments, R⁶ is

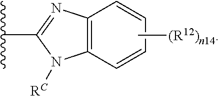

In some embodiments, R⁶ is

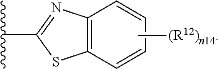

In an aspect, the structural lipid of the invention features a compound having the structure of Formula SII:

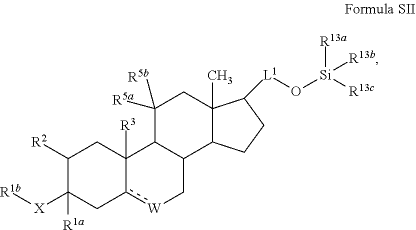

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

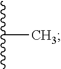

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to

which each is attached, combine to form

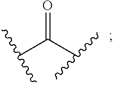 [SMILES: *C(*)=O]

L¹ is optionally substituted C₁-C₆ alkylene; and

each of R^{13a}, R^{13b}, and R^{13c} is, independently, optionally substituted C₁-C₆ alkyl or optionally substituted C₆-C₁₀ aryl,

or a pharmaceutically acceptable salt thereof.

In some embodiments, the compound has the structure of Formula SIIa:

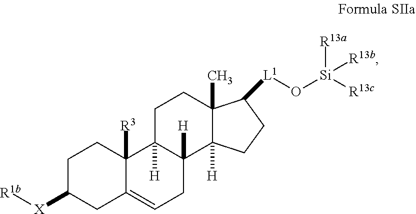

or a pharmaceutically acceptable salt thereof.

In some embodiments, the compound has the structure of Formula SIIb:

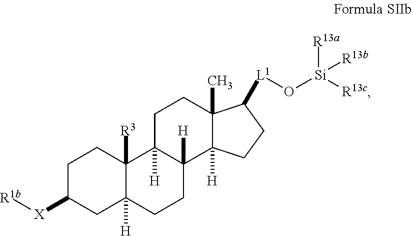

or a pharmaceutically acceptable salt thereof.

In some embodiments, L¹ is

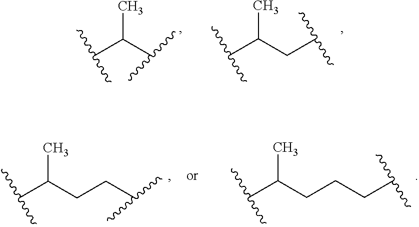

In some embodiments, each of R^{13a}, R^{13b}, and R^{13c} is, independently,

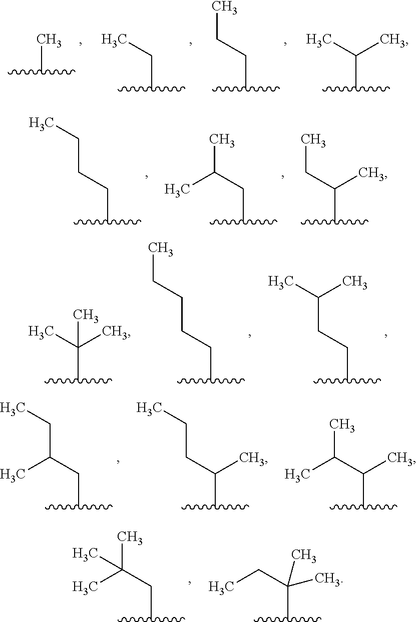

In an aspect, the structural lipid of the invention features a compound having the structure of Formula SIII:

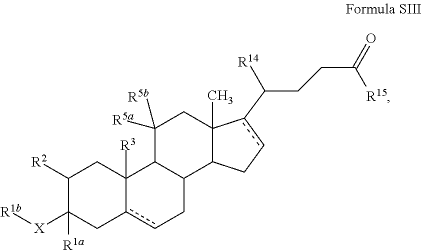

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

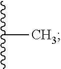

each [symbol] independently represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, hydroxyl, optionally substituted C₁-C₆ alkyl, —OS(O)₂R^{4c}, where R^{4c}, is optionally substituted C₁-C₆ alkyl or optionally substituted C₆-C₁₀ aryl;

each of R^{5a} and R^{5b} is, independently, H or OR^{A}, or R^{5a} and R^{5b}, together with the atom to which each is attached, combine to form

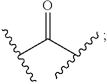 [SMILES: *C(*)=O]

R¹⁴ is H or C₁-C₆ alkyl; and

R¹⁵ is

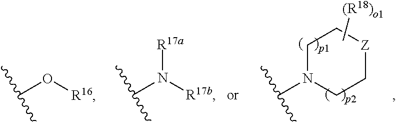

where

R¹⁶ is H or optionally substituted C₁-C₆ alkyl;
R^{17b} is H, OR^{17c}, optionally substituted C₆-C₁₀ aryl, or optionally substituted C₁-C₆alkyl;

R^{17c} is H or optionally substituted C₁-C₆ alkyl;

o1 is 0, 1, 2, 3, 4, 5, 6, 7, or 8;

p1 is 0, 1, or 2;

p2 is 0, 1, or 2;

Z is CH₂ 0, S, or NR^{D}, where R^{D} is H or optionally substituted C₁-C₆ alkyl; and

each R¹⁸ is, independently, halo or optionally substituted C₁-C₆ alkyl, or a pharmaceutically acceptable salt thereof.

In some embodiments, the compound has the structure of Formula SIIIa:

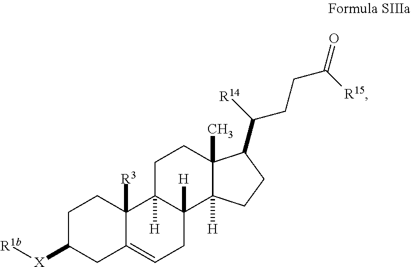

or a pharmaceutically acceptable salt thereof.

In some embodiments, the compound has the structure of Formula SIIIb:

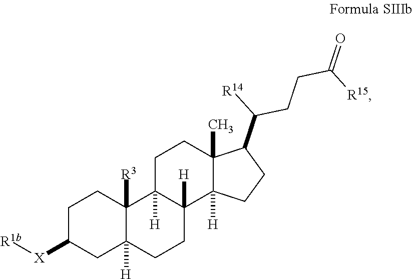

or a pharmaceutically acceptable salt thereof.

In some embodiments, R¹⁴ is H,

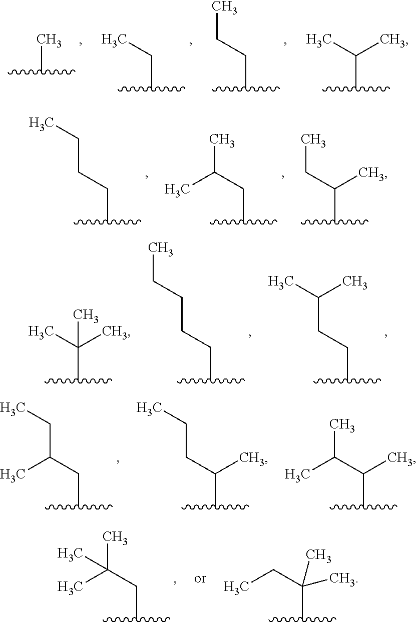

In some embodiments, R¹⁴ is

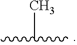

In some embodiments, R¹⁵ is

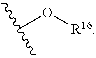

In some embodiments, R¹⁵ is

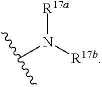

In some embodiments, R¹⁶ is H. In some embodiments, R¹⁶ is

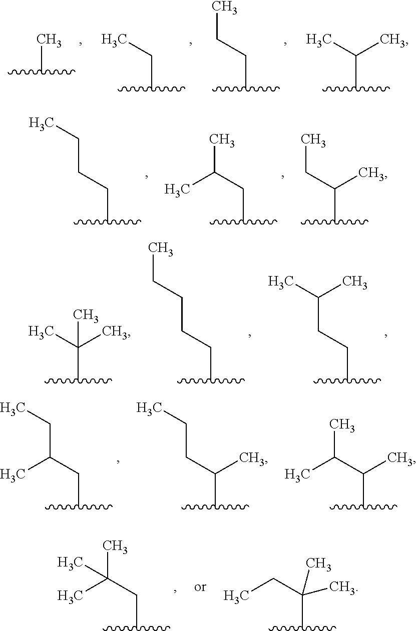

In some embodiments, R^{17a} is H. In some embodiments, R^{17a} is optionally substituted C₁-C₆alkyl.

In some embodiments, R^{17b} is H. In some embodiments, R^{17b} optionally substituted C₁-C₆alkyl. In some embodiments, R^{17b} is OR^{17c}.

In some embodiments, R^{17c} is H,

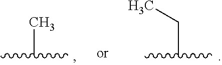

In some embodiments, R^{17c} is H. In some embodiments, R^{17c} is

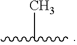

In some embodiments, R¹⁵ is

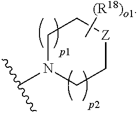

In some embodiments, each R¹⁸ is, independently,

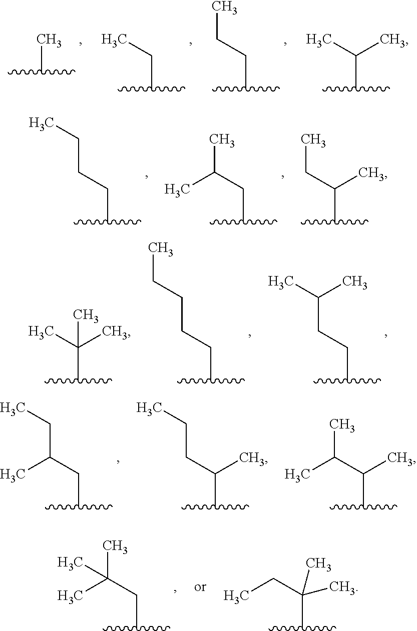

In some embodiments, Z is CH₂. In some embodiments, Z is O. In some embodiments, Z is NR^{D}.

In some embodiments, o1 is 0, 1, 2, 3, 4, 5, or 6.

In some embodiments, o1 is 0. In some embodiments, o1 is 1. In some embodiments, o1 is 2. In some embodiments, o1 is 3. In some embodiments, o1 is 4. In some embodiments, o1 is 5. In some embodiments, o1 is 6.

In some embodiments, p1 is 0 or 1. In some embodiments, p1 is 0. In some embodiments, p1 is 1.

In some embodiments, p2 is 0 or 1. In some embodiments, p2 is 0. In some embodiments, p2 is 1.

In an aspect, the structural lipid of the invention features a compound having the structure of Formula SIV:

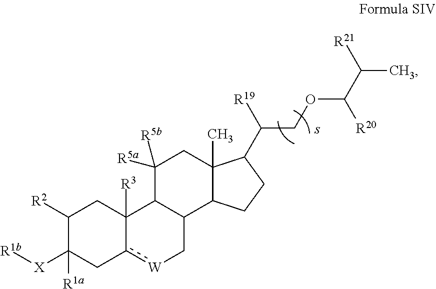

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

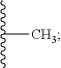

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to

which each is attached, combine to form

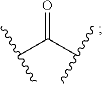 [SMILES: *C(*)=O]

s is 0 or 1;

R¹⁹ is H or C₁-C₆ alkyl;

R²⁰ is C₁-C₆ alkyl;

R²¹ is H or C₁-C₆ alkyl,

or a pharmaceutically acceptable salt thereof.

In some embodiments, the compound has the structure of Formula SIVa:

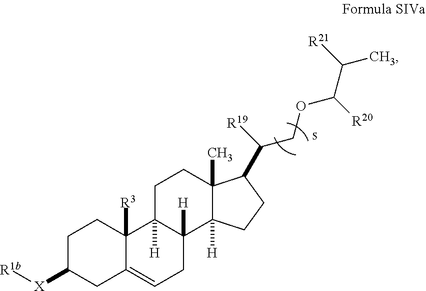

or a pharmaceutically acceptable salt thereof.

In some embodiments, the compound has the structure of Formula SIVb:

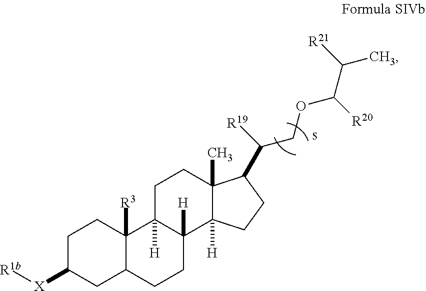

or a pharmaceutically acceptable salt thereof.

In some embodiments, R¹⁹ is H,

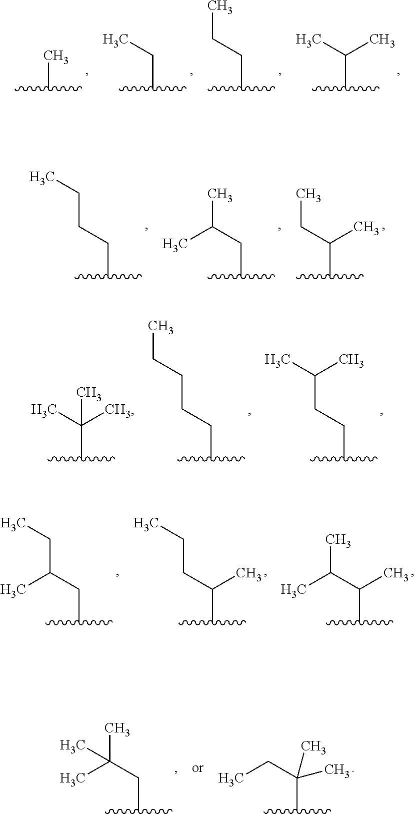

In some embodiments, R¹⁹ is

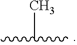

In some embodiments, R²⁰ is,

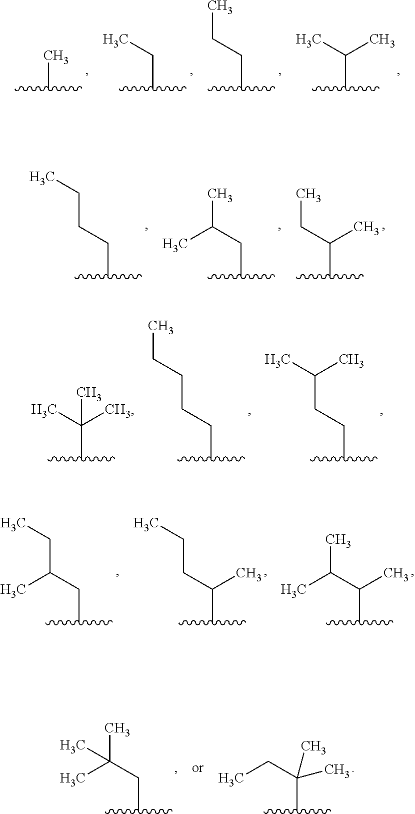

In some embodiments, R²¹ is H,

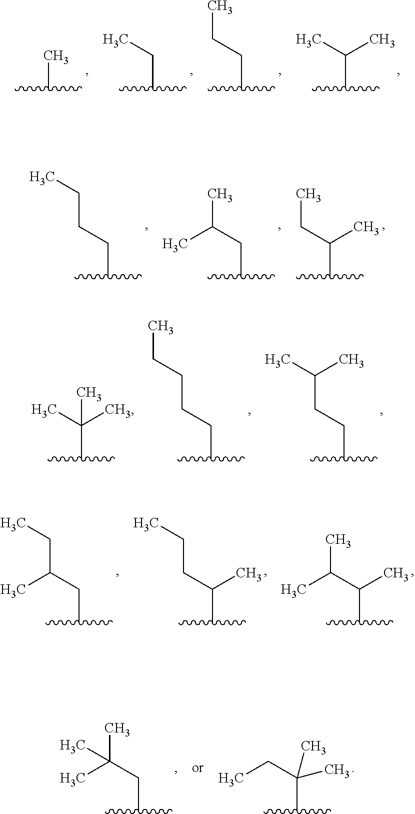

In an aspect, the structural lipid of the invention features, a compound having the structure of Formula SV:

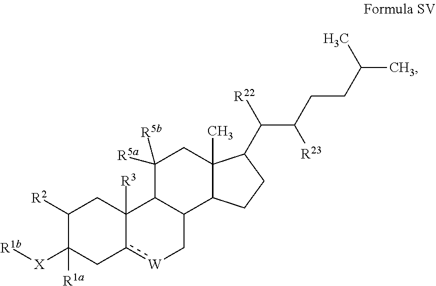

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

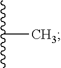

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to

which each is attached, combine to form

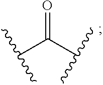 [SMILES: *C(*)=O]

R²² is H or C₁-C₆ alkyl; and

R²³ is halo, hydroxyl, optionally substituted C₁-C₆ alkyl, or optionally substituted C₁-C₆ heteroalkyl,

or a pharmaceutically acceptable salt thereof.

In some embodiments, the compound has the structure of Formula SVa:

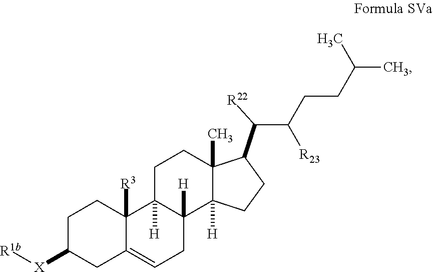

or a pharmaceutically acceptable salt thereof.

In some embodiments, the compound has the structure of Formula SVb:

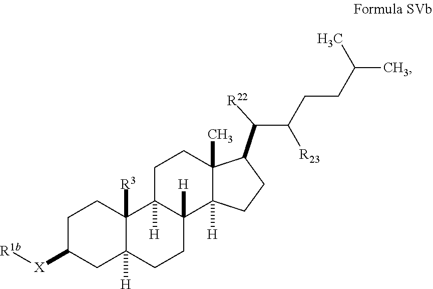

or a pharmaceutically acceptable salt thereof.
In some embodiments, R²² is H,

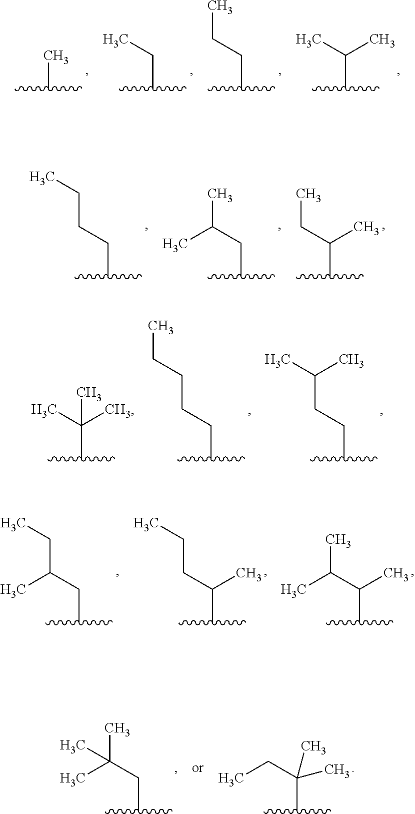

In some embodiments, R²² is

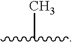

In some embodiments, R²³ is

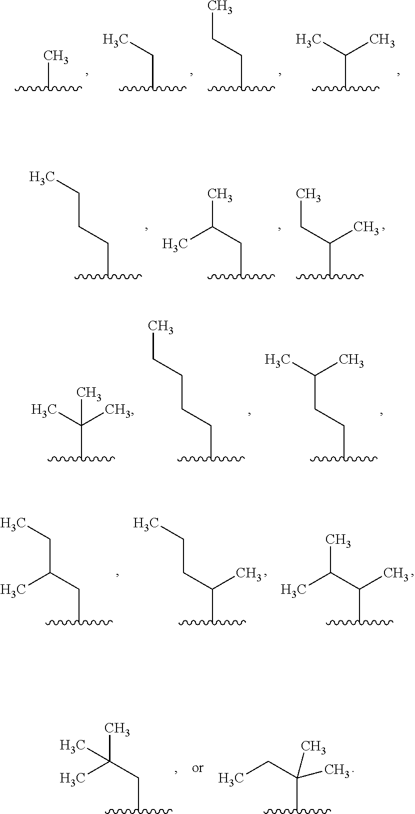

In an aspect, the structural lipid of the invention features a compound having the structure of Formula SVI:

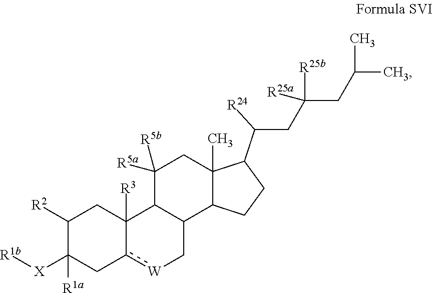

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

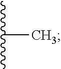

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to

which each is attached, combine to form

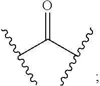 [SMILES: *C(*)=O]

R²⁴ is H or C₁-C₆ alkyl; and

each of R^{25a} and R^{25b} is C₁-C₆ alkyl,

or a pharmaceutically acceptable salt thereof.

In some embodiments, the compound has the structure of Formula SVIa:

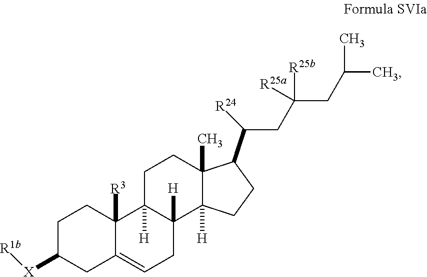

or a pharmaceutically acceptable salt thereof.

In some embodiments, the compound has the structure of Formula SVIb:

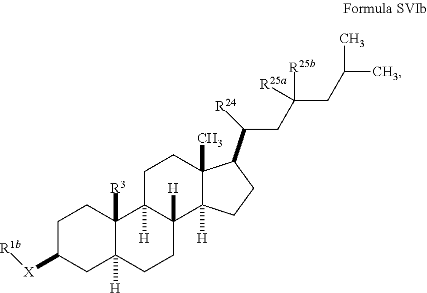

or a pharmaceutically acceptable salt thereof.

In some embodiments, R²⁴ is H,

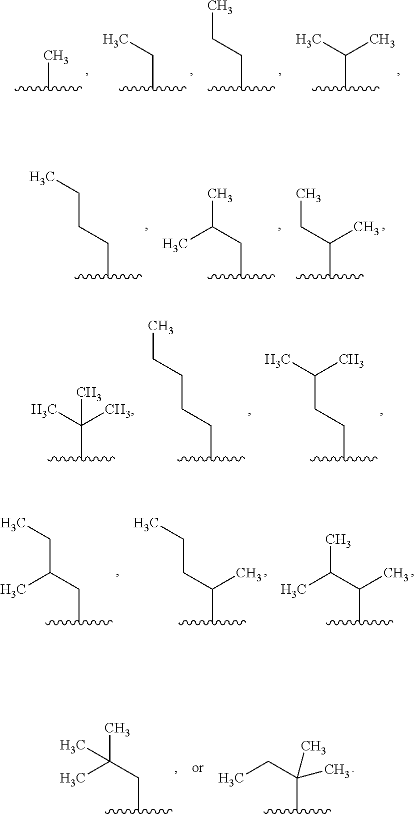

In some embodiments, R²⁴ is

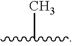

In some embodiments, each of R^{25a} and R^{25b} is, independently,

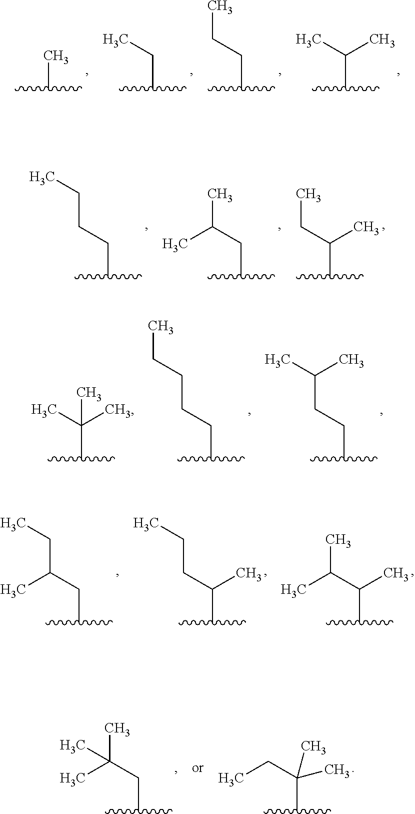

In an aspect, the structural lipid of the invention features a compound having the structure of Formula SVII:

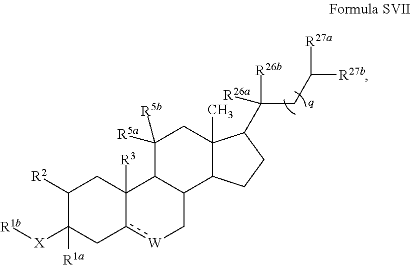

where

R^{1a} is H, optionally substituted C₁-C₆ alkyl, optionally substituted C₂-C₆ alkenyl, optionally substituted C₂-C₆ alkynyl, or

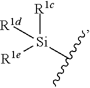

where each of R^{1c}, R^{1d}, and R^{1e} is, independently, optionally substituted C₁-C₆ alkyl or optionally substituted C₆-C₁₀ aryl;

X is O or S;

R^{1b} is H or optionally substituted C₁-C₆ alkyl;

R² is H or OR^{A}, where R^{A} is H or optionally substituted C₁-C₆ alkyl;

R³ is H or

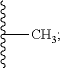

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

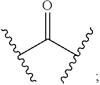 [SMILES: *C(*)=O]

q is 0 or 1;

each of R^{26a} and R^{26b} is, independently, H or optionally substituted C₁-C₆ alkyl, or R^{26a} and R^{26b}, together with the atom to which each is attached, combine to form or

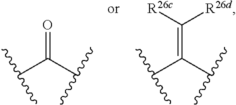

where each of R^{26c} and R²⁶ is, independently, H or optionally substituted C₁-C₆ alkyl; and

each of R^{27a} and R^{27b} is H, hydroxyl, or optionally substituted C₁-C₆ alkyl, or a pharmaceutically acceptable salt thereof.

In some embodiments, the compound has the structure of Formula SVIIa:

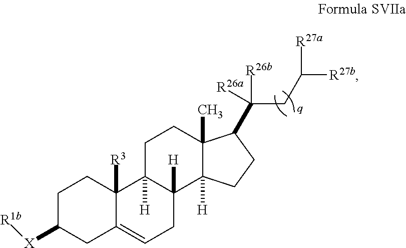

or a pharmaceutically acceptable salt thereof.

In some embodiments, the compound has the structure of Formula SVIIb:

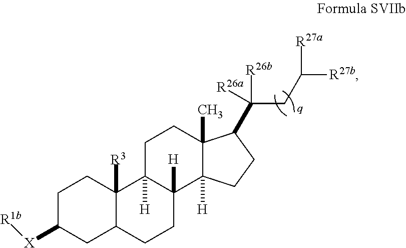

or a pharmaceutically acceptable salt thereof.

In some embodiments, R^{26a} and R^{26b} is, independently, H,

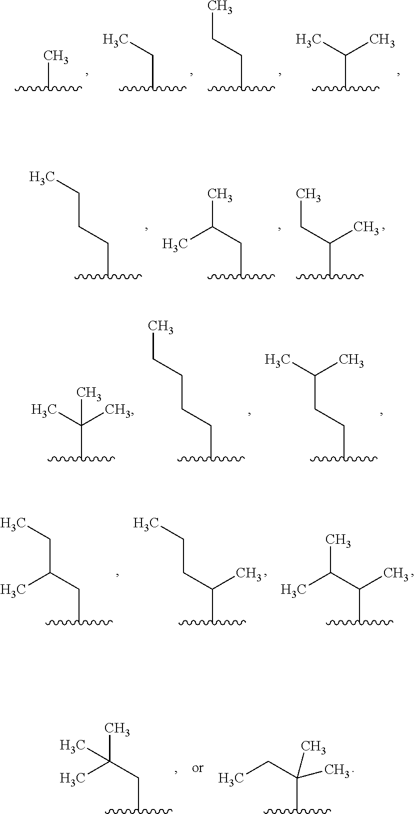

In some embodiments, R^{26a} and R^{26b}, together with the atom to which each is attached, combine to form

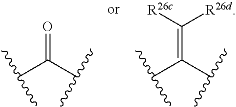

In some embodiments, R^{26a} and R^{26b}, together with the atom to which each is attached, combine to form

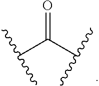 [SMILES: *C(*)=O]

In some embodiments, R^{26a} and R^{26b}, together with the atom to which each is attached, combine to form

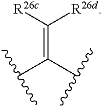

In some embodiments, where each of R^{26c} and R²⁶ is, independently, H,

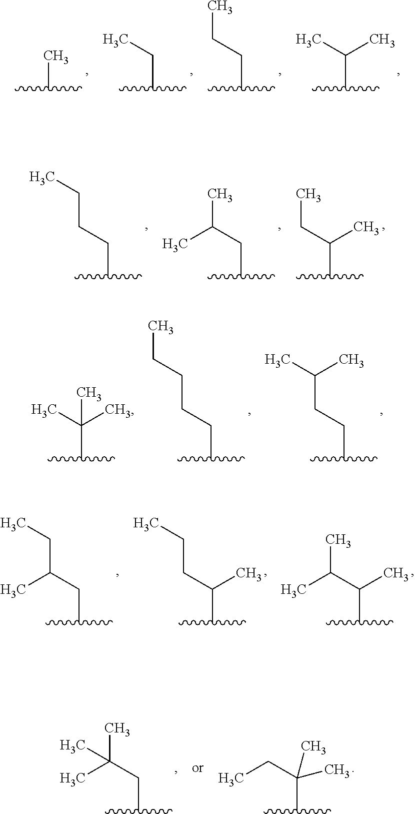

In some embodiments, each of R^{27a} and R^{27b} is H, hydroxyl, or optionally substituted C₁-C₃alkyl.

In some embodiments, each of R^{27a} and R^{27b} is, independently, H, hydroxyl,

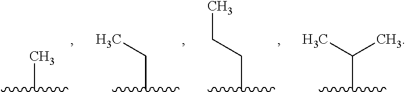

In an aspect, the structural lipid of the invention features a compound having the structure of Formula SVIII:

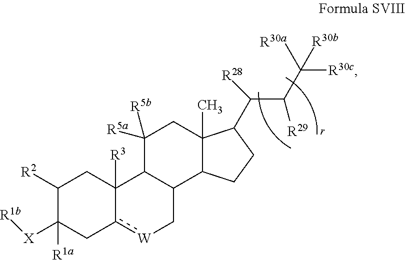

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

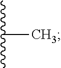

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

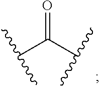 [SMILES: *C(*)=O]

R²⁸ is H or optionally substituted C₁-C₆ alkyl;

r is 1, 2, or 3;

each R²⁹ is, independently, H or optionally substituted C₁-C₆ alkyl; and

each of R^{30a}, R^{30b}, and R^{30c} is C₁-C₆ alkyl,

or a pharmaceutically acceptable salt thereof.

In some embodiments, the compound has the structure of Formula SVIIIa:

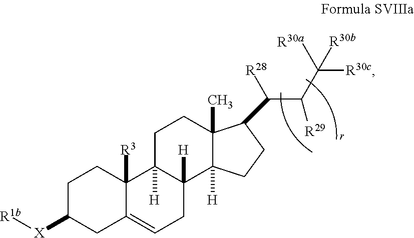

or a pharmaceutically acceptable salt thereof.

In some embodiments, the compound has the structure of Formula SVIIIb:

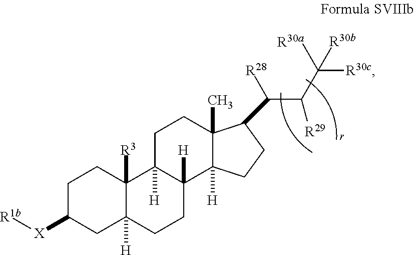

or a pharmaceutically acceptable salt thereof.

In some embodiments, R²⁸ is H,

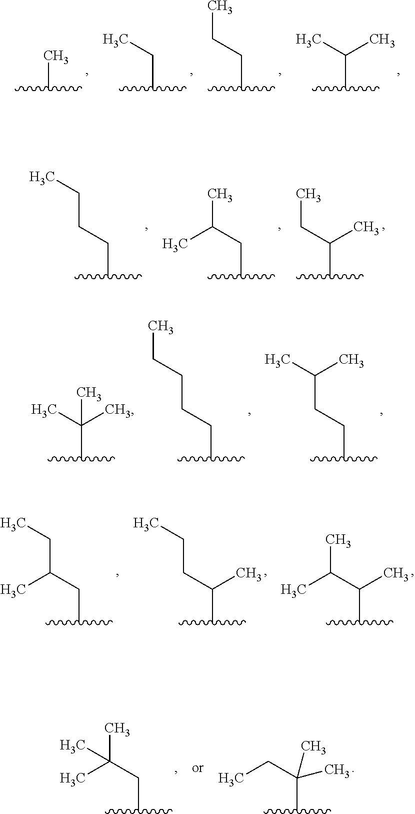

In some embodiments, R²⁸ is

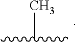

In some embodiments, each of R^{30a}, R^{30b}, and R^{30c} is, independently,

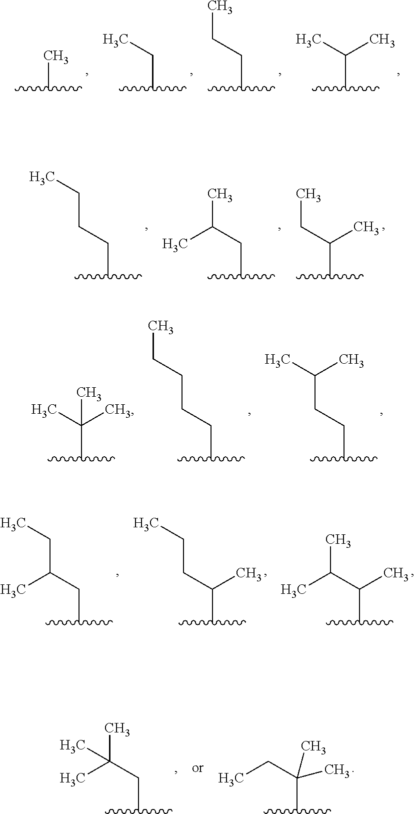

In some embodiments, r is 1. In some embodiments, r is 2. In some embodiments, r is 3.

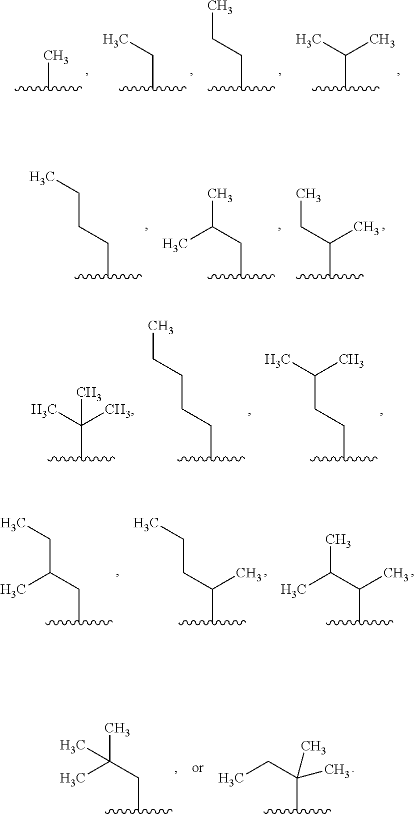

In some embodiments, each R²⁹ is, independently, H,

In some embodiments, each R²⁹ is, independently, H or

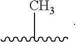

In an aspect, the structural lipid of the invention features a compound having the structure of Formula SIX:

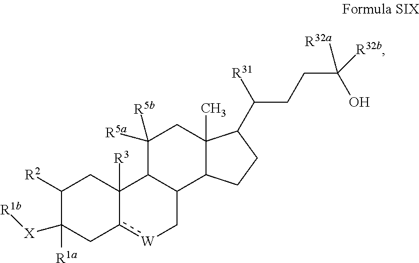

where

R^{1a} is H, optionally substituted C₁-C₆ alkyl, optionally substituted C₂-C₆ alkenyl, or optionally substituted C₂-C₆ alkynyl;

X is O or S;

R^{1b} is H or optionally substituted C₁-C₆ alkyl;

R² is H or OR^{A}, where R^{A} is H or optionally substituted C₁-C₆ alkyl;

R³ is H or

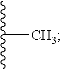

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

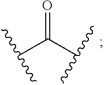 [SMILES: *C(*)=O]

R³¹ is H or C₁-C₆ alkyl; and

each of R^{32a} and R^{32b} is C₁-C₆ alkyl,

or a pharmaceutically acceptable salt thereof.

In some embodiments, the compound has the structure of Formula SIXa:

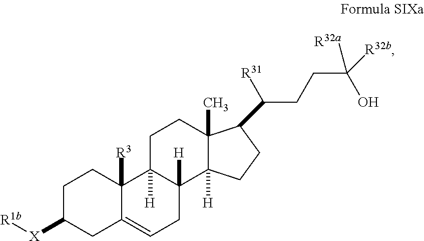

or a pharmaceutically acceptable salt thereof.

In some embodiments, the compound has the structure of Formula SIXb:

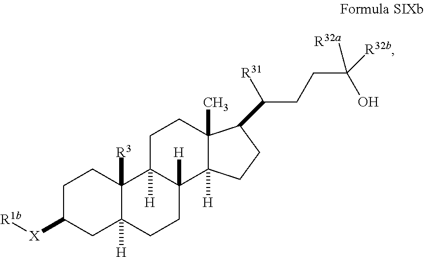

or a pharmaceutically acceptable salt thereof.

In some embodiments, R³¹ is H,

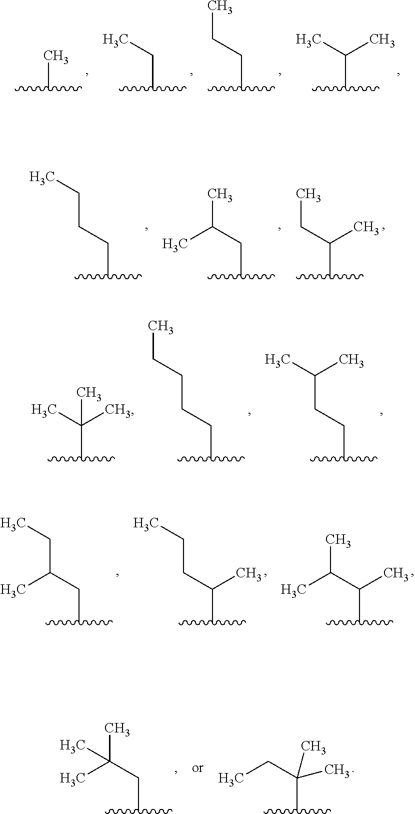

In some embodiments, R³¹ is

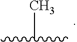

In some embodiments, each of R^{32a} and R^{32b} is, independently,

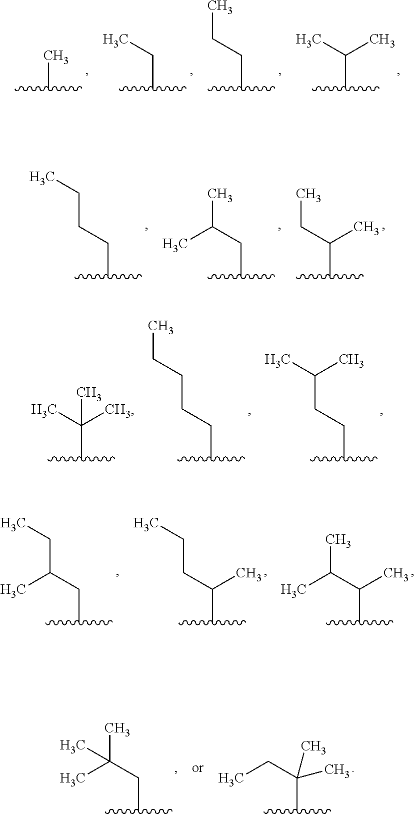

In an aspect, the structural lipid of the invention features a compound having the structure of Formula SX:

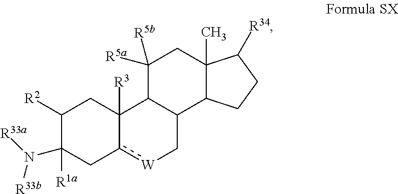

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

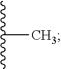

[symbol] R represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

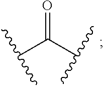 [SMILES: *C(*)=O]

R^{33a} is optionally substituted C₁-C₆ alkyl or

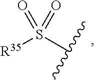

where R³⁵ is optionally substituted C₁-C₆ alkyl or optionally substituted C₆-C₁₀ aryl;

R^{33b} is H or optionally substituted C₁-C₆ alkyl; or

R³⁵ and R^{33b}, together with the atom to which each is attached, form an optionally substituted C₃-C₉ heterocyclyl; and

R³⁴ is optionally substituted C₁-C₆ alkyl or optionally substituted C₁-C₆ heteroalkyl, or a pharmaceutically acceptable salt thereof.

In some embodiments, the compound has the structure of Formula SXa:

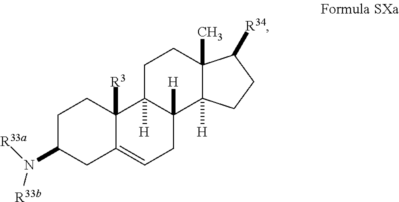

or a pharmaceutically acceptable salt thereof.

In some embodiments, the compound has the structure of Formula SXb:

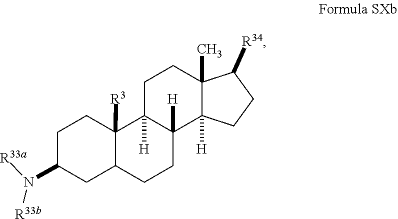

or a pharmaceutically acceptable salt thereof.

In some embodiments, R^{33a} is

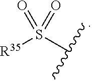

In some embodiments, R³⁵ is

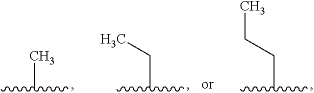

In some embodiments, R³⁵ is

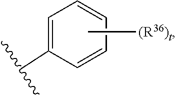

where

t is 0, 1, 2, 3, 4, or 5; and

each R³⁶ is, independently, halo, hydroxyl, optionally substituted C₁-C₆ alkyl, or optionally substituted C₁-C₆ heteroalkyl.

In some embodiments, R³⁴ is

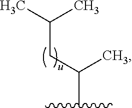

where u is 0, 1, 2, 3, or 4.

In some embodiments, u is 3 or 4.

In an aspect, the structural lipid of the invention features a compound having the structure of Formula SXI:

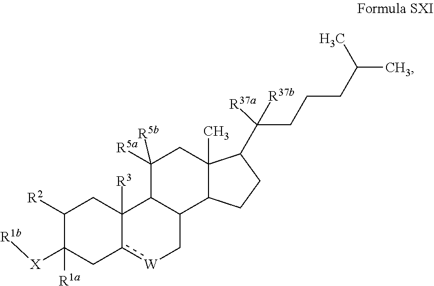

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

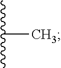

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

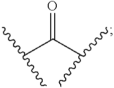 [SMILES: *C(*)=O]

and

each of R^{37a} and R^{37b} is, independently, optionally substituted C₁-C₆ alkyl, optionally substituted C₁-C₆ heteroalkyl, halo, or hydroxyl,

or a pharmaceutically acceptable salt thereof.

In some embodiments, the compound has the structure of Formula SXIa:

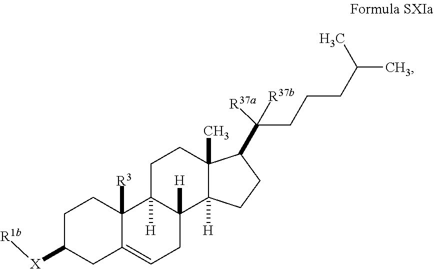

or a pharmaceutically acceptable salt thereof.

In some embodiments, the compound has the structure of Formula SXIb:

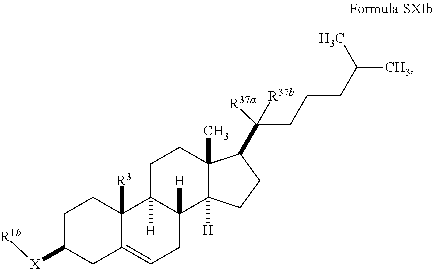

or a pharmaceutically acceptable salt thereof.

In some embodiments, R^{37a} is hydroxyl.

In some embodiments, R^{37b} is

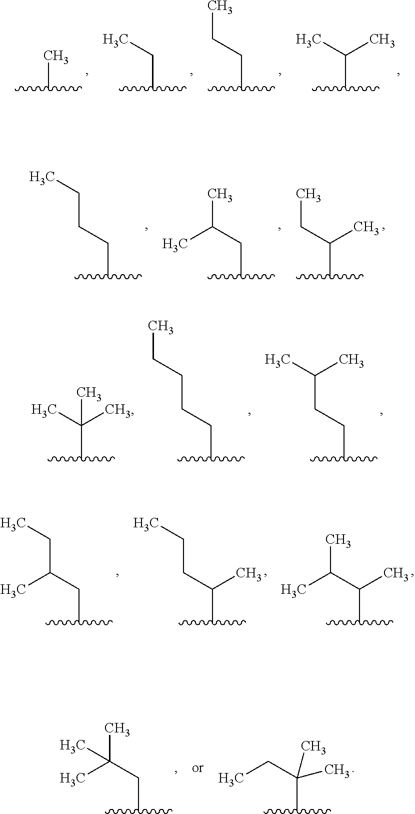

In an aspect, the structural lipid of the invention features a compound having the structure of Formula SXII:

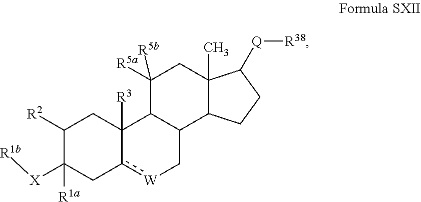

where

R^{1a} is H, optionally substituted C₁-C₆ alkyl, optionally substituted C₂-C₆ alkenyl, or optionally substituted C₂-C₆ alkynyl;

X is O or S;

R² is H or OR^{A}, where R^{A} is H or optionally substituted C₁-C₆ alkyl;

R³ is H or

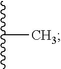

[symbol] represents a single bond or a double bond;

W is CR^{4a} or CR^{4a}R^{4b}, where if a double bond is present between W and the adjacent carbon, then W is CR^{4a}; and if a single bond is present between W and the adjacent carbon, then W is CR^{4a}R^{4b};

each of R^{4a} and R^{4b} is, independently, H, halo, or optionally substituted C₁-C₆ alkyl;

each of R^{5a} and R^{5b} is, independently, H or OR^{A}, or R^{5a} and R^{5b}, together with the atom to which each is attached, combine to form

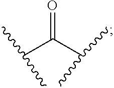 [SMILES: *C(*)=O]

and

Q is O, S, or NR^{E}, where R^{E} is H or optionally substituted C₁-C₆ alkyl; and

R³⁸ is optionally substituted C₁-C₆ alkyl,

or a pharmaceutically acceptable salt thereof.

In some embodiments, the compound has the structure of Formula SXIIa:

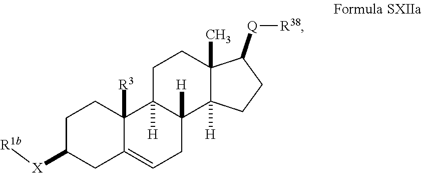

or a pharmaceutically acceptable salt thereof.

In some embodiments, the compound has the structure of Formula SXIIb:

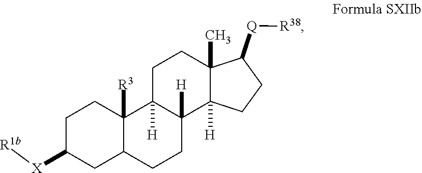

or a pharmaceutically acceptable salt thereof.

In some embodiments, Q is NR^{E}.

In some embodiments, R^{E} is H or

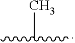

In some embodiments, R^{E} is H. In some embodiments, R^{E} is

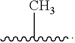

In some embodiments, R³⁸ is

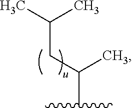

where u is 0, 1, 2, 3, or 4.

In some embodiments, X is O.

In some embodiments, R^{1a} is H or optionally substituted C₁-C₆ alkyl.

In some embodiments, R^{1a} is H.

In some embodiments, R^{1b} is H or optionally substituted C₁-C₆ alkyl.

In some embodiments, Rib is H.

In some embodiments, R² is H.

In some embodiments, R^{4a} is H.

In some embodiments, R^{4b} is H.

In some embodiments, [symbol] represents a double bond.

In some embodiments, R³ is H. In some embodiments, R³ is

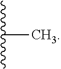

In some embodiments, R^{5a} is H.

In some embodiments, R^{5b} is H.

In an aspect, the invention features a compound having the structure of any one of compounds S-1-42, S-150, S-154, S-162-165, S-169-172 and S-184 in Table 1, or any pharmaceutically acceptable salt thereof. As used herein, “CMPD” refers to “compound.”

TABLE 1
Compounds of Formula SI

| CMPD | |
|---|---|
| No. S- | Structure |
| 1 | 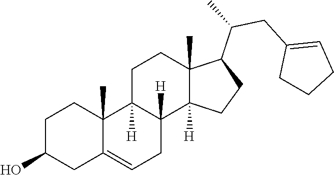 |
| 2 | 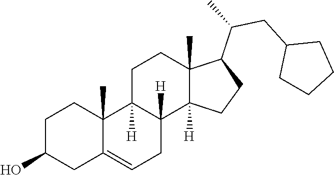 |
| 3 | 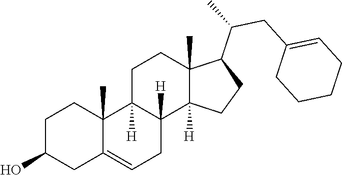 |
| 4 | 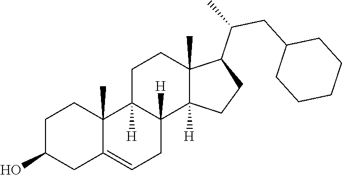 |
| 5 | 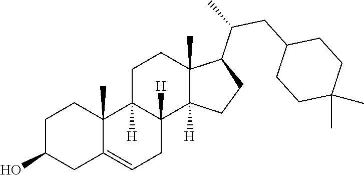 |
| 6 | 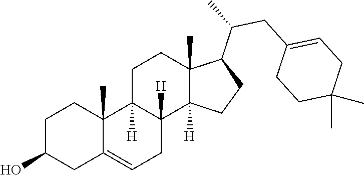 |
| 7 | 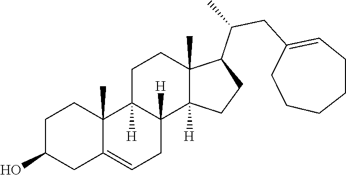 |
| 8 | 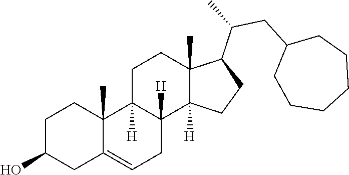 |
| 9 | 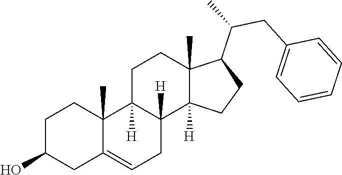 |
| 10 | 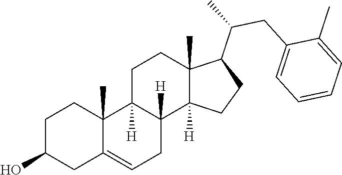 |
| 11 | 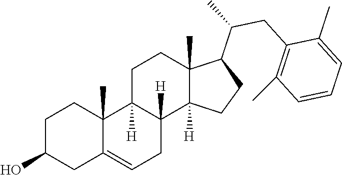 |
| 12 | 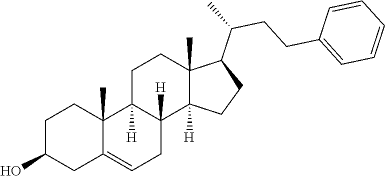 |
| 13 | 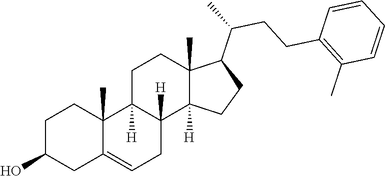 |
| 14 | 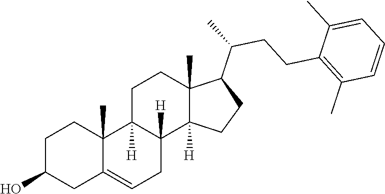 |
| 15 | 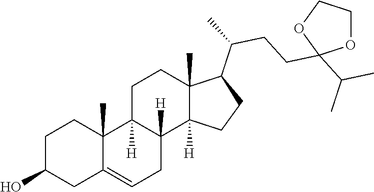 |
| 16 | 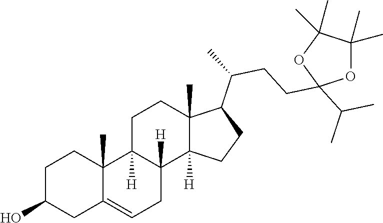 |
| 17 | 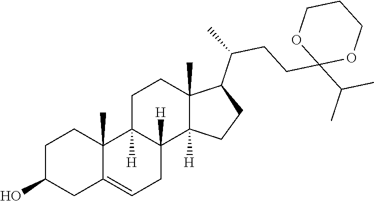 |
| 18 | 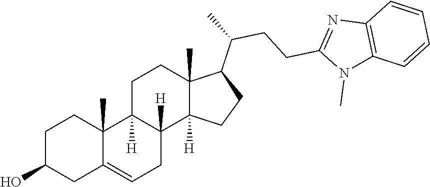 |
| 19 | 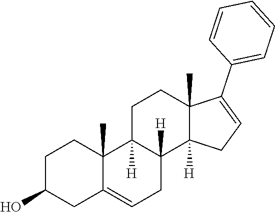 |
| 20 | 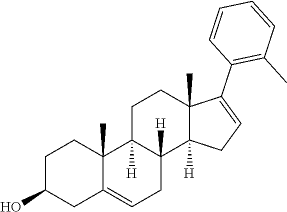 |
| 21 | 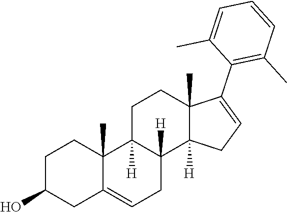 |
| 150 | 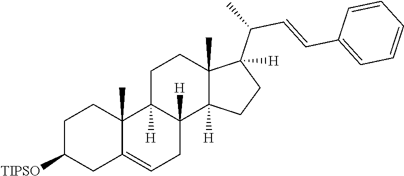 |
| 154 | 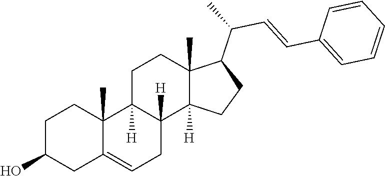 |
| 162 | 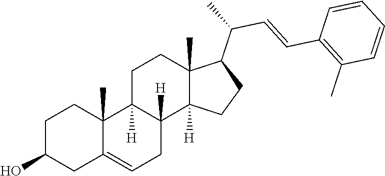 |
| 163 | 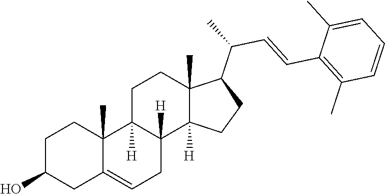 |
| 164 | 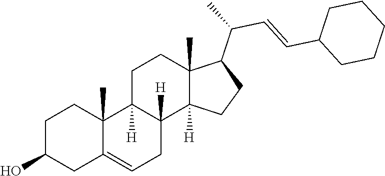 |
| 184 | 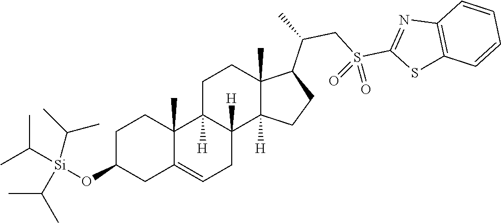 |
| 22 | 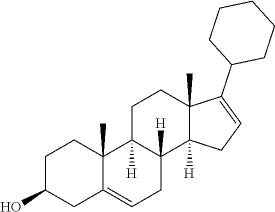 |
| 23 | 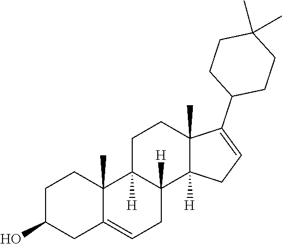 |
| 24 | 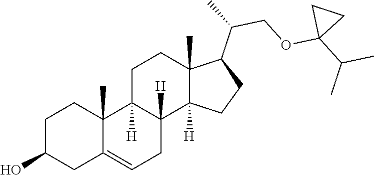 |
| 25 | 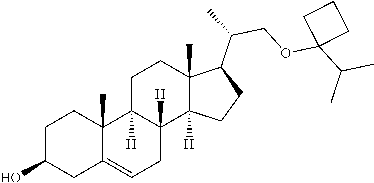 |
| 26 | 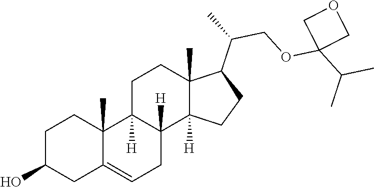 |
| 27 | 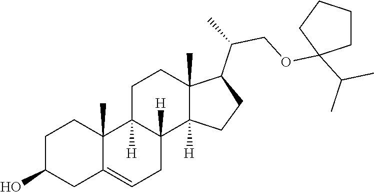 |
| 28 | 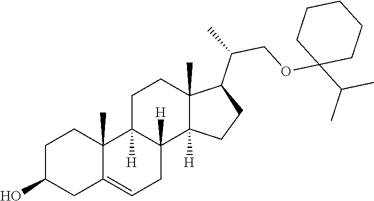 |
| 29 | 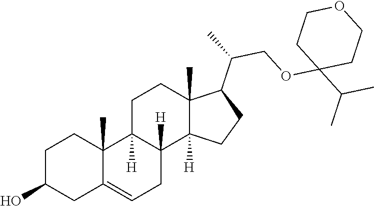 |
| 30 | 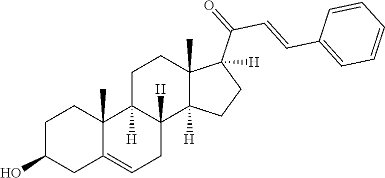 |
| 31 | 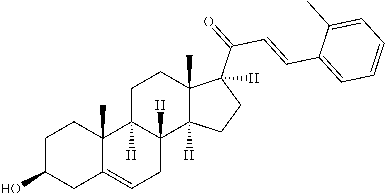 |
| 32 | 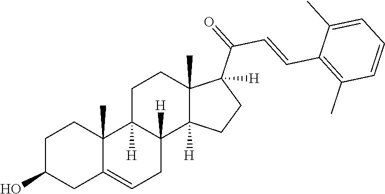 |
| 33 | 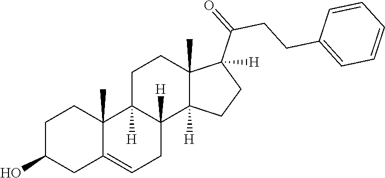 |
| 34 | 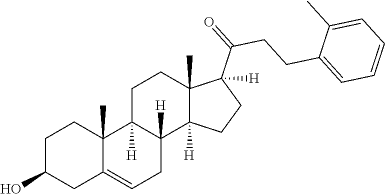 |
| 35 | 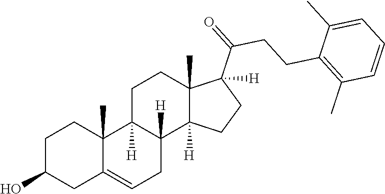 |
| 36 | 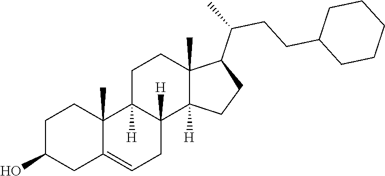 |
| 37 | 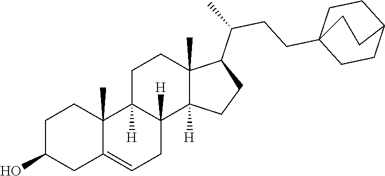 |
| 38 | 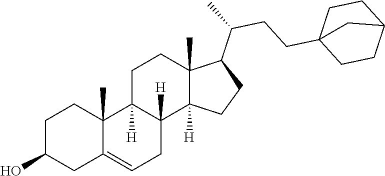 |
| 39 | 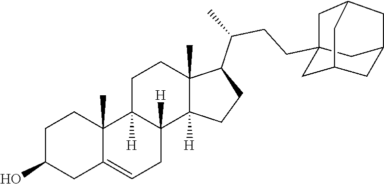 |
| 40 | 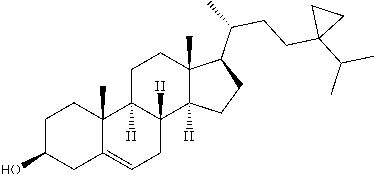 |
| 41 | 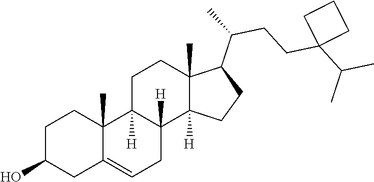 |
| 42 | 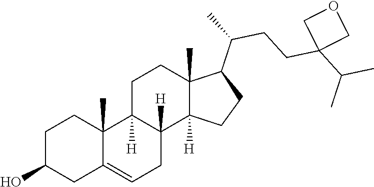 |
| 165 | 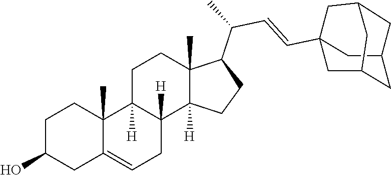 |
| 169 | 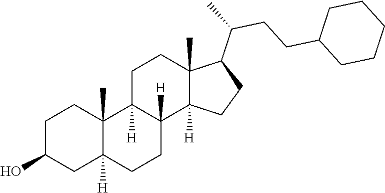 |
| 170 | 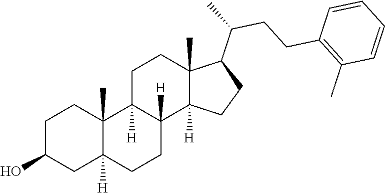 |
| 171 | 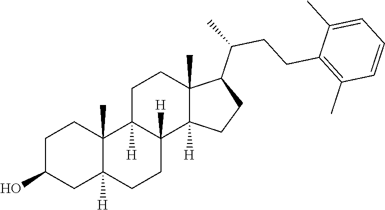 |
| 172 | 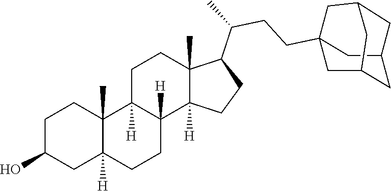 |

In an aspect, the invention features a compound having the structure of any one of compounds S-43-50 and S-175-178 in Table 2, or any pharmaceutically acceptable salt thereof.

TABLE 2
Compounds of Formula SII

| CMPD No. S— | Structure |
|---|---|
| 43 | 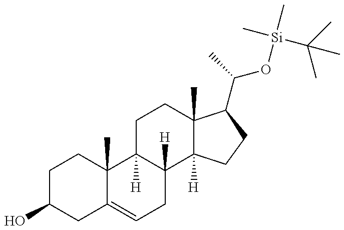 |
| 44 | 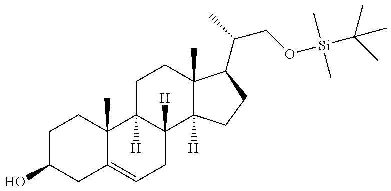 |
| 45 | 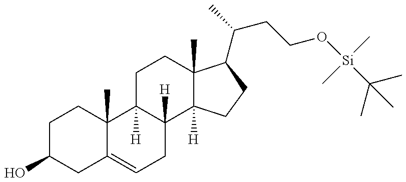 |
| 46 | 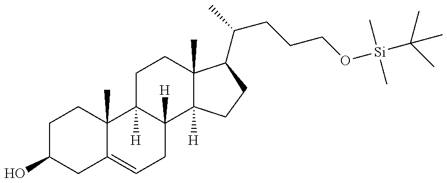 |
| 175 | 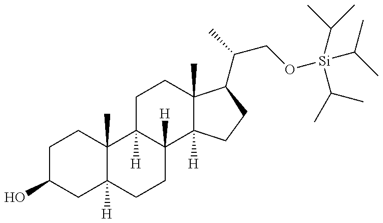 |
| 176 | 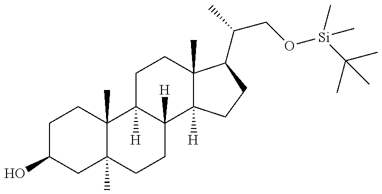 |
| 47 | 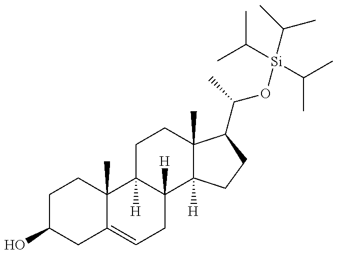 |
| 48 | 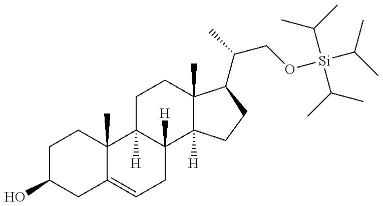 |
| 49 | 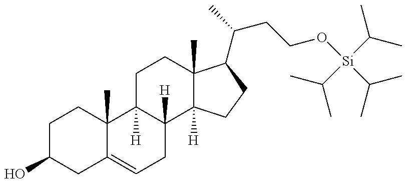 |
| 50 | 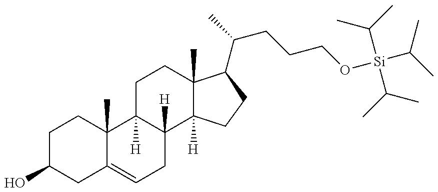 |
| 177 | 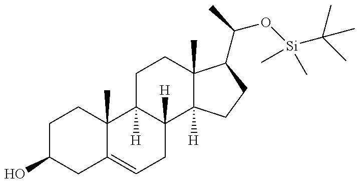 |
| 178 | 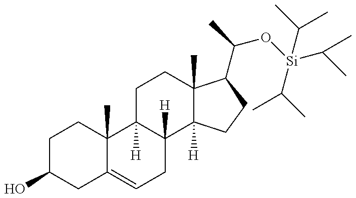 |

In an aspect, the invention features a compound having the structure of any one of compounds S-51-67, S-149 and S-153 in Table 3, or any pharmaceutically acceptable salt thereof.

TABLE 3
Compounds of Formula SIII

| CMPD No. S— | Structure |
|---|---|
| 51 | 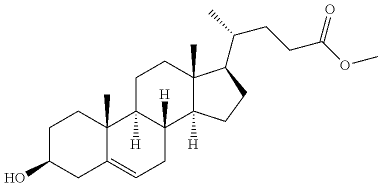 |
| 52 | 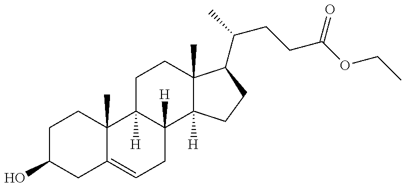 |
| 53 | 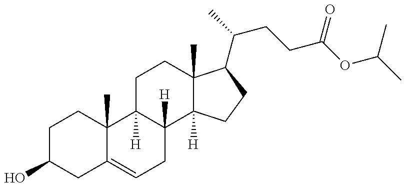 |
| 54 | 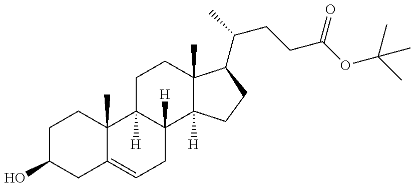 |
| 55 | 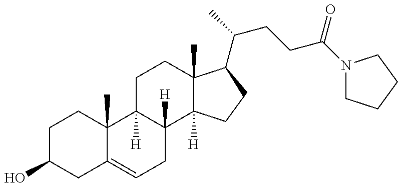 |
| 56 | 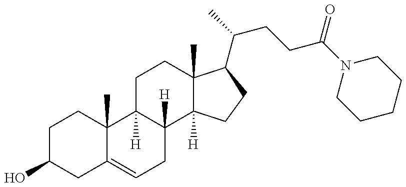 |
| 57 | 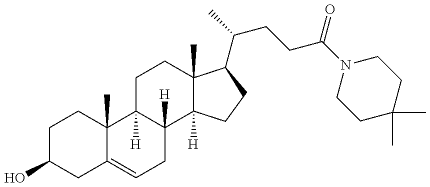 |
| 58 | 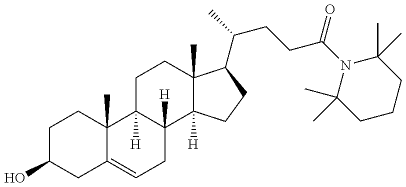 |
| 59 | 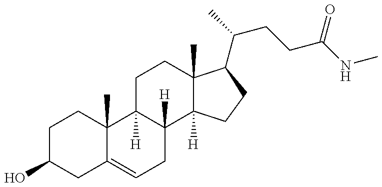 |
| 153 | 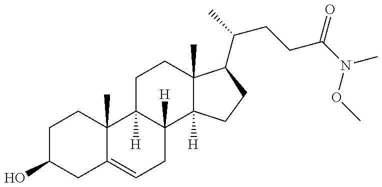 |
| 60 | 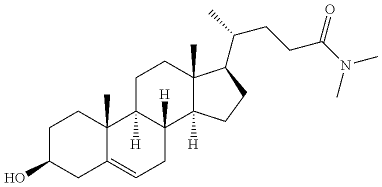 |
| 61 | 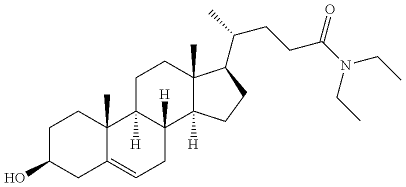 |
| 62 | 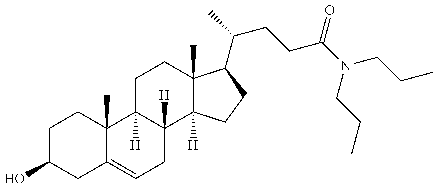 |
| 63 | 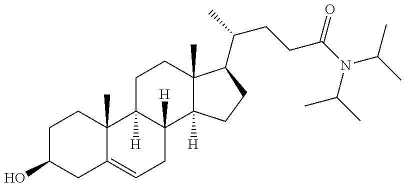 |
| 64 | 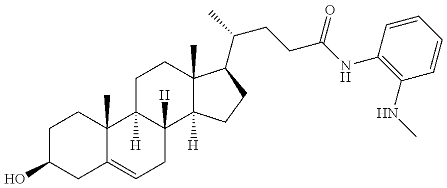 |
| 65 | 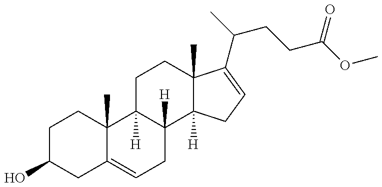 |
| 66 | 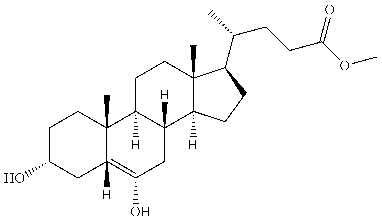 |
| 67 | 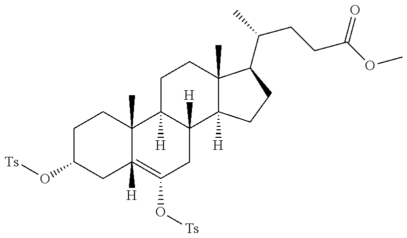 |
| 149 | 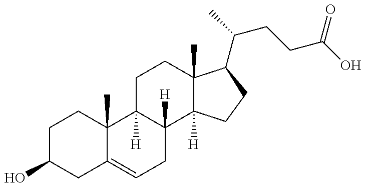 |

In an aspect, the invention features a compound having the structure of any one of compounds S-68-73 in Table 4, or any pharmaceutically acceptable salt thereof.

TABLE 4
Compounds of Formula SIV

| CMPD | |
|---|---|
| No. S— | Structure |
| 68 | 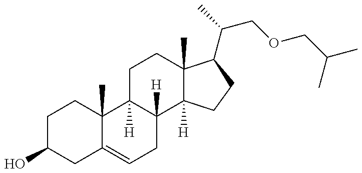 |
| 69 | 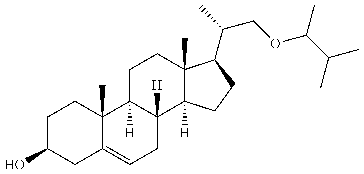 |
| 70 | 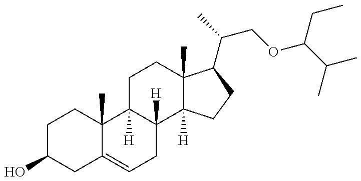 |
| 71 | 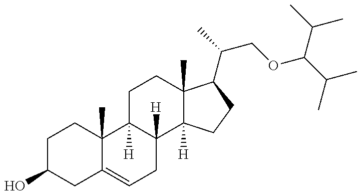 |
| 72 | 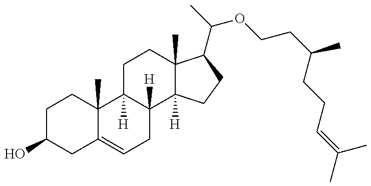 |
| 73 | 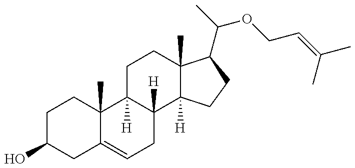 |

In an aspect, the invention features a compound having the structure of any one of compounds S-74-78 in Table 5, or any pharmaceutically acceptable salt thereof.

TABLE 5
Compounds of Formula SV

| CMPD | |
|---|---|
| No. S— | Structure |
| 74 | 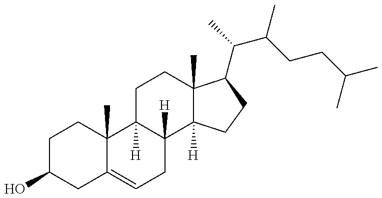 |
| 75 | 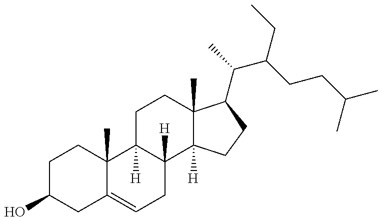 |
| 76 | 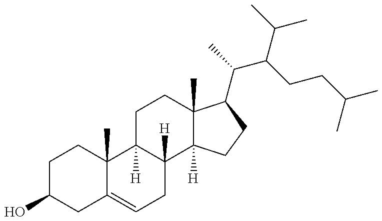 |
| 77 | 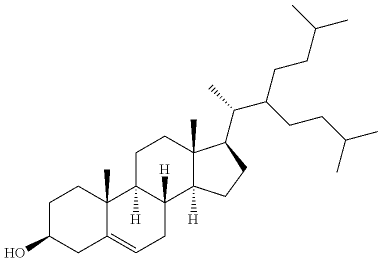 |
| 78 | 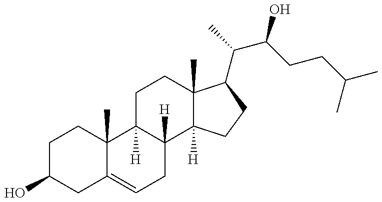 |

In an aspect, the invention features a compound having the structure of any one of compounds S-79 or S-80 in Table 6, or any pharmaceutically acceptable salt thereof.

TABLE 6
Compounds of Formula SVI

| CMPD | |
|---|---|
| No. S— | Structure |
| 79 | 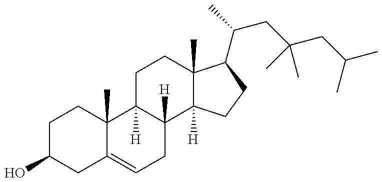 |
| 80 | 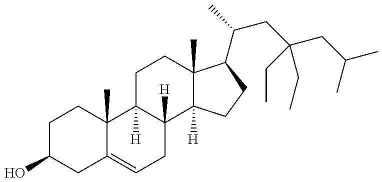 |

In an aspect, the invention features a compound having the structure of any one of compounds S-81-87, S-152 and S-157 in Table 7, or any pharmaceutically acceptable salt thereof.

TABLE 7
Compounds of Formula S-VII

| CMPD | |
|---|---|
| No. S— | Structure |
| 81 | 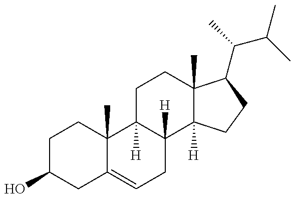 |
| 82 | 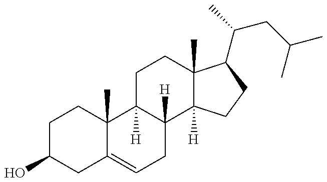 |
| 83 | 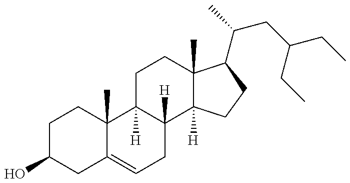 |
| 84 | 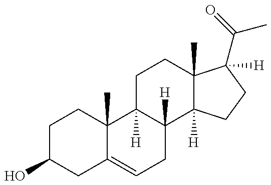 |
| 157 | 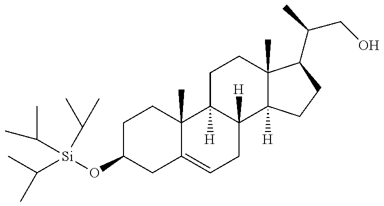 |
| 85 | 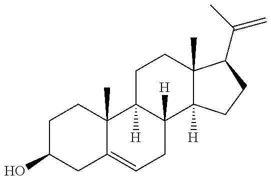 |
| 86 | 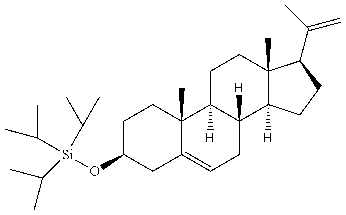 |
| 87 | 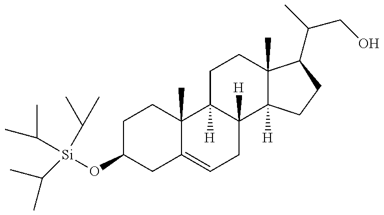 |
| 152 | 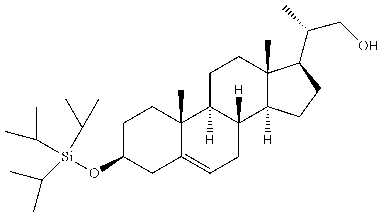 |

In an aspect, the invention features a compound having the structure of any one of compounds S-88-97 in Table 8, or any pharmaceutically acceptable salt thereof.

TABLE 8
Compounds of Formula SVIII

| CMPD | |
|---|---|
| No. S— | Structure |
| 88 | 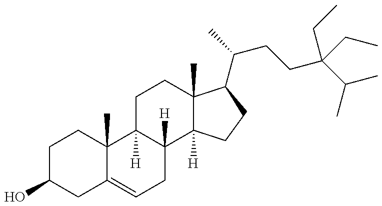 |
| 89 | 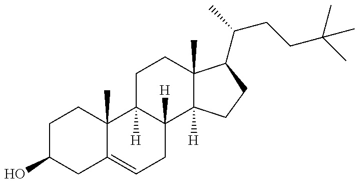 |
| 90 | 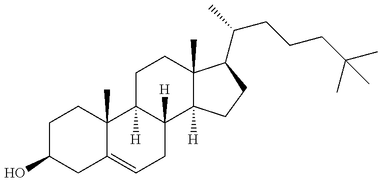 |
| 91 | 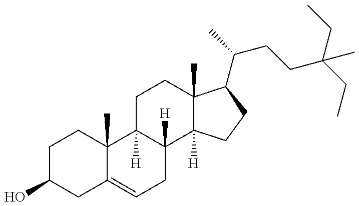 |
| 92 | 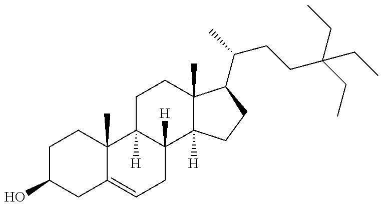 |
| 93 | 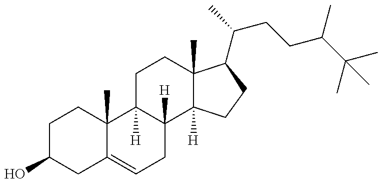 |
| 94 | 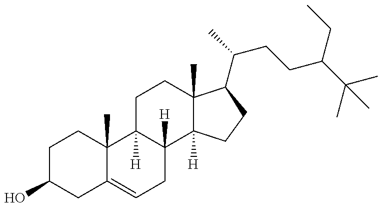 |
| 95 | 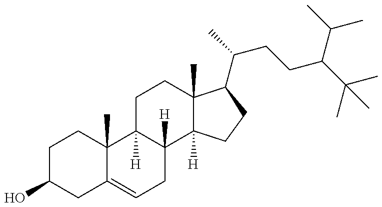 |
| 96 | 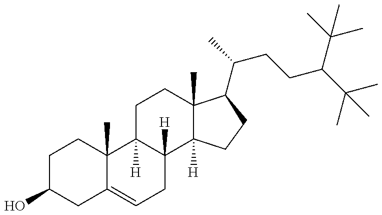 |
| 97 | 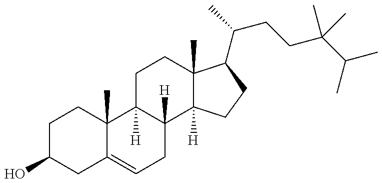 |

In an aspect, the invention features a compound having the structure of any one of compounds S-98-105 and S-180-182 in Table 9, or any pharmaceutically acceptable salt thereof.

TABLE 9
Compounds of Formula SIX

| Cmpd | |
|---|---|
| No. S— | Structure |
| 98 | 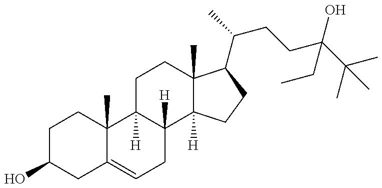 |
| 99 | 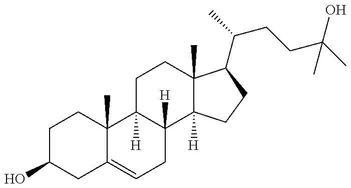 |
| 100 | 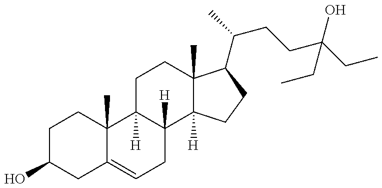 |
| 101 | 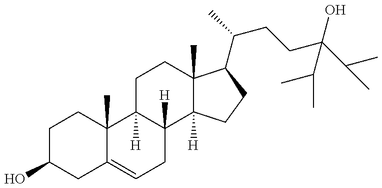 |
| 180 | 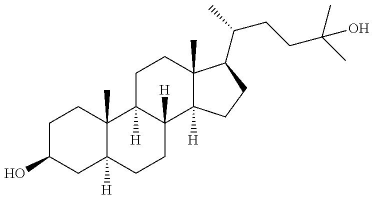 |
| 181 | 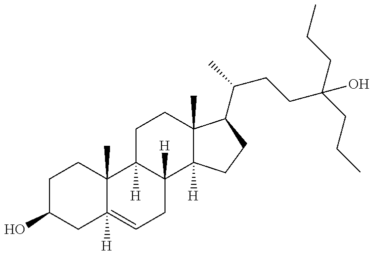 |
| 102 | 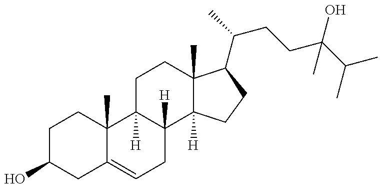 |
| 103 | 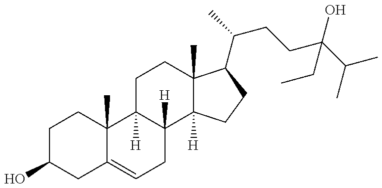 |
| 104 | 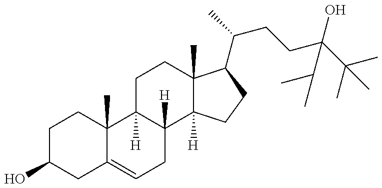 |
| 105 | 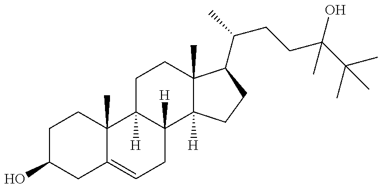 |
| 182 | 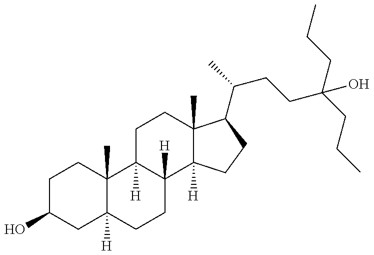 |

In an aspect, the invention features a compound having the structure of compound S-106 in Table 10, or any pharmaceutically acceptable salt thereof.

TABLE 10
Compounds of Formula SX

| CMPD | |
|---|---|
| No. S— | Structure |
| 106 | 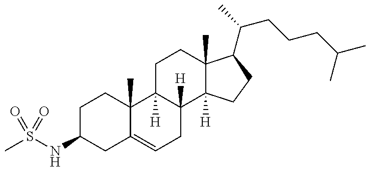 |

In an aspect, the invention features a compound having the structure of compound S-107 or S-108 in Table 11, or any pharmaceutically acceptable salt thereof.

TABLE 11
Compounds of Formula SXI

| CMPD | |
|---|---|
| No. S— | Structure |
| 107 | 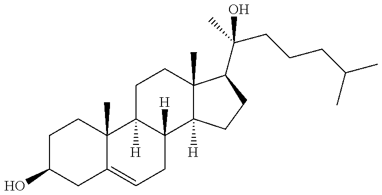 |
| 108 | 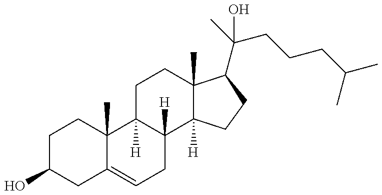 |

In an aspect, the invention features a compound having the structure of compound S-109 in Table 12, or any pharmaceutically acceptable salt thereof.

TABLE 12
Compounds of Formula SXII

| CMPD | |
|---|---|
| No. S— | Structure |
| 109 | 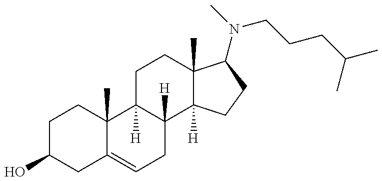 |

In an aspect, the invention features a compound having the structure of any one of compounds S-110-130, S-155, S-156, S-158, S-160, S-161, S-166-168, S-173, S-174 and S-179 in Table 13, or any pharmaceutically acceptable salt thereof.

TABLE 13
Compounds of the Invention

| CMPD No. S— | Structure |
|---|---|
| 110 | 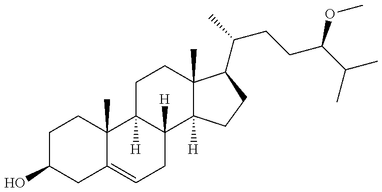 |
| 111 | 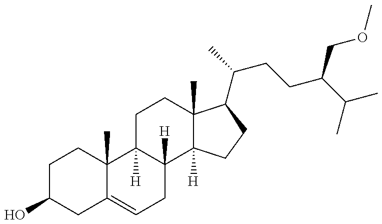 |
| 112 | 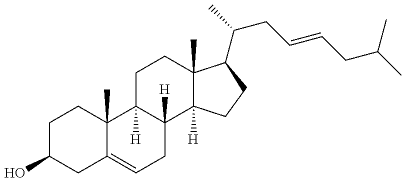 |
| 113 | 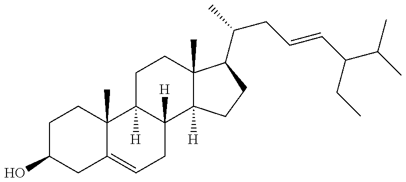 |
| 114 | 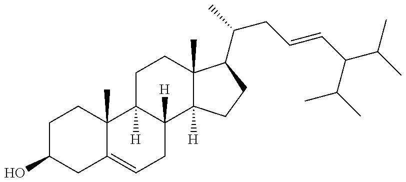 |
| 115 | 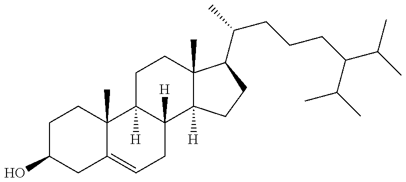 |
| 116 | 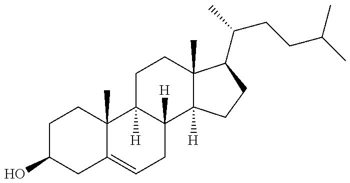 |
| 117 | 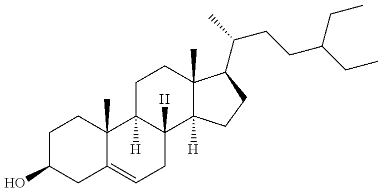 |
| 118 | 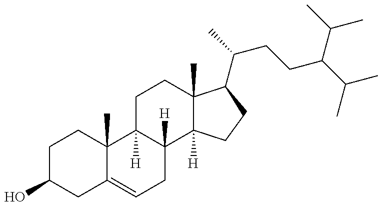 |
| 119 | 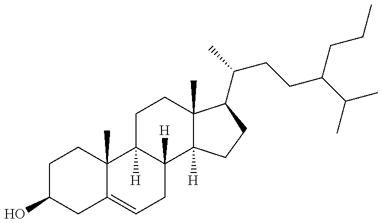 |
| 120 | 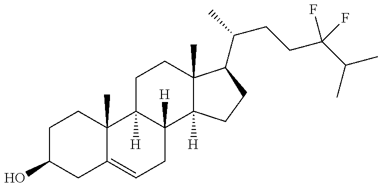 |
| 156 | 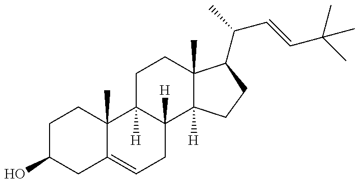 |
| 158 | 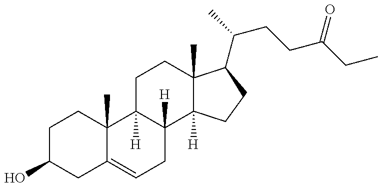 |
| 160 | 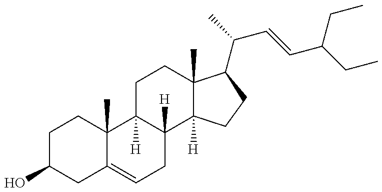 |
| 161 | 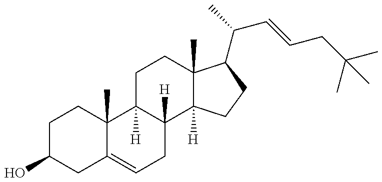 |
| 166 | 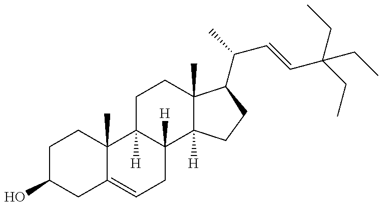 |
| 121 | 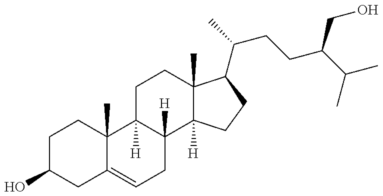 |
| 122 | 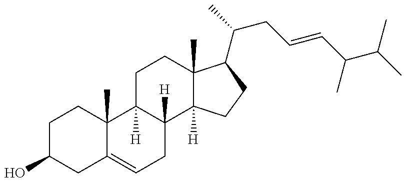 |
| 123 | 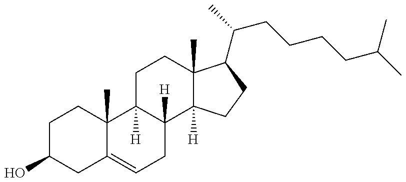 |
| 124 | 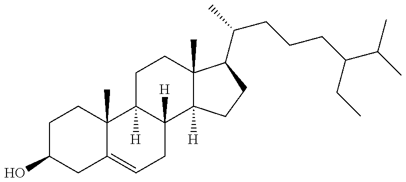 |
| 125 | 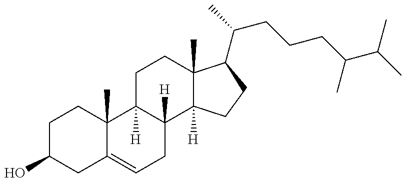 |
| 126 | 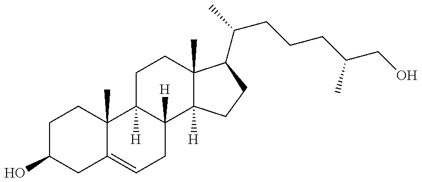 |
| 127 | 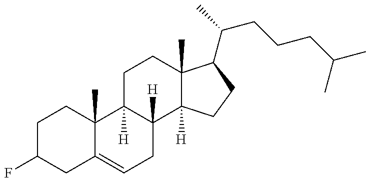 |
| 128 | 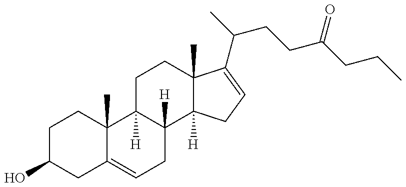 |
| 129 | 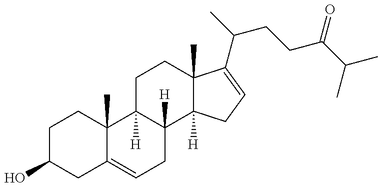 |
| 130 | 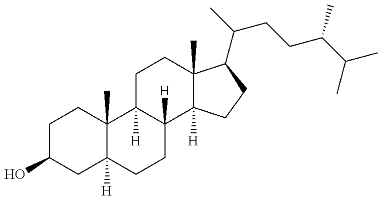 |
| 155 | 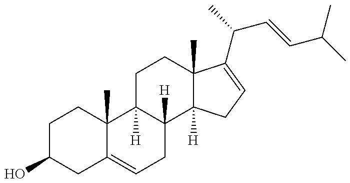 |
| 167 | 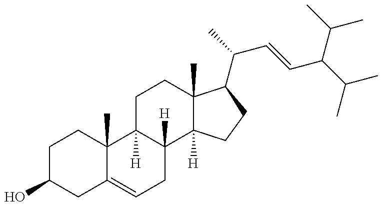 |
| 168 | 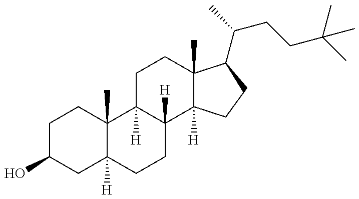 |
| 173 | 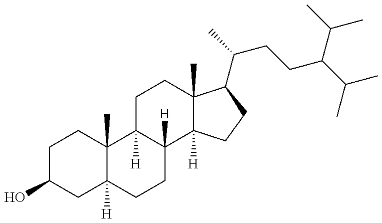 |
| 174 | 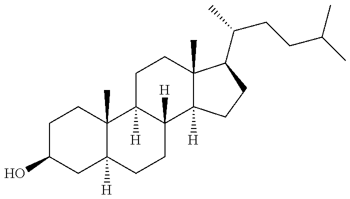 |
| 179 | 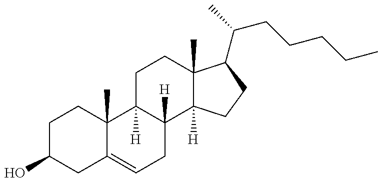 |

In an aspect, the invention features a compound having the structure of any one of compounds S-131-133 in Table 14, or any pharmaceutically acceptable salt thereof.

TABLE 14
Compounds of the Invention

| CMPD | |
|---|---|
| No. S— | Structure |
| 131 | 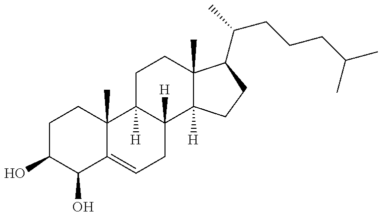 |
| 132 | 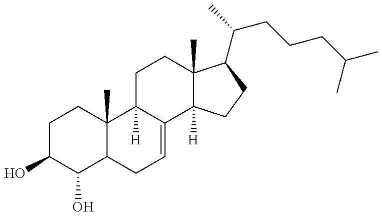 |
| 133 | 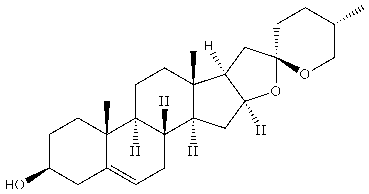 |

In an aspect, the invention features a compound having the structure of any one of compounds S-134-148, S-151 and S-159 in Table 15, or any pharmaceutically acceptable salt thereof.

TABLE 15
Compounds of the Invention

| CMPD | |
|---|---|
| No. S— | Structure |
| 134 | 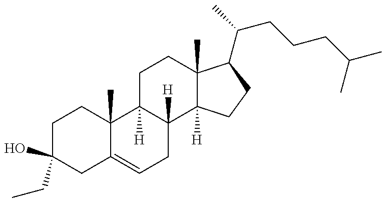 |
| 135 | 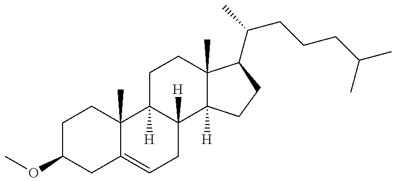 |
| 136 | 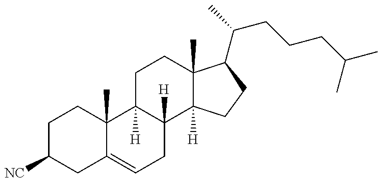 |
| 137 | 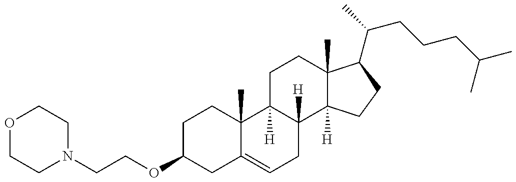 |
| 138 | 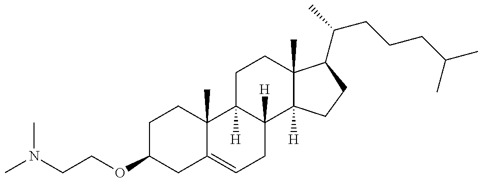 |
| 139 | 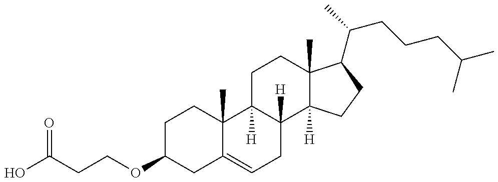 |
| 140 | 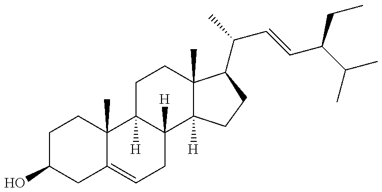 |
| 141 | 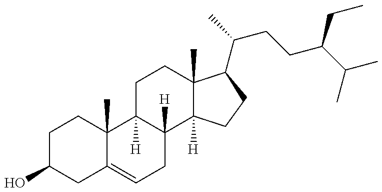 |
| 159 | 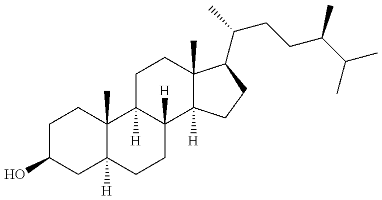 |
| 142 | 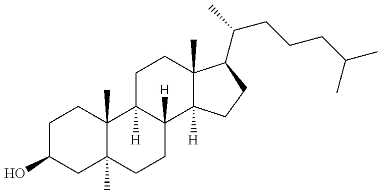 |
| 143 | 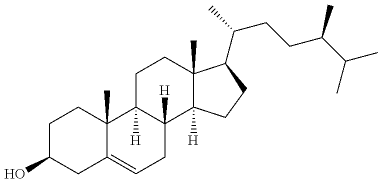 |
| 144 | 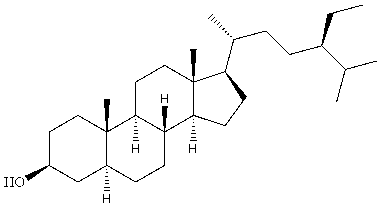 |
| 145 | 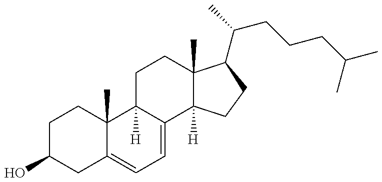 |
| 146 | 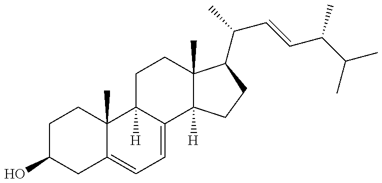 |
| 147 | 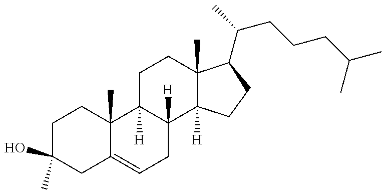 |
| 148 | 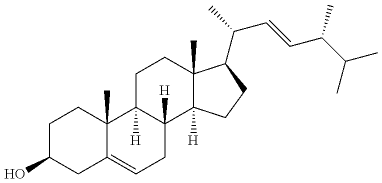 |
| 151 | 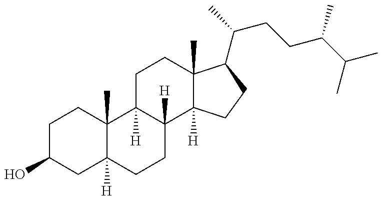 |

The one or more structural lipids of the lipid nanoparticles of the invention can be a composition of structural lipids (e.g., a mixture of two or more structural lipids, a mixture of three or more structural lipids, a mixture of four or more structural lipids, or a mixture of five or more structural lipids). A composition of structural lipids can include, but is not limited to, any combination of sterols (e.g., cholesterol, β-sitosterol, fecosterol, ergosterol, sitosterol, campesterol, stigmasterol, brassicasterol, ergosterol, tomatidine, tomatine, ursolic acid, alpha-tocopherol, or any one of compounds 134-148, 151, and 159 in Table 15). For example, the one or more structural lipids of the lipid nanoparticles of the invention can be composition 183 in Table 16.

TABLE 16
Structural Lipid Compositions

| Composi- | |
|---|---|
| tion | |
| S— No. | Structure |
| 183 | 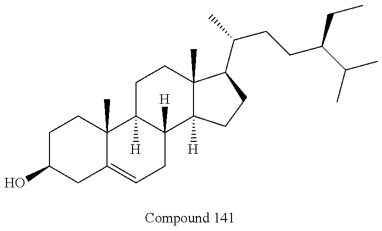 |
| | 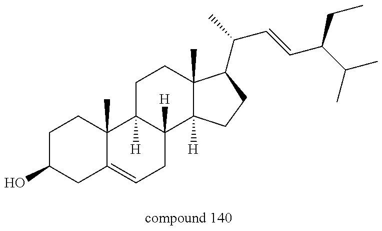 |
| | 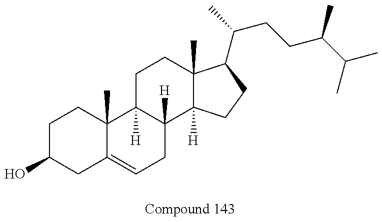 |
| | 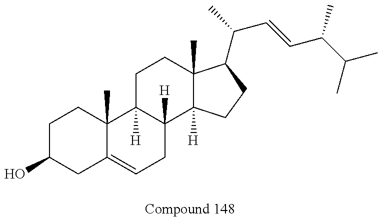 |

Composition S-183 is a mixture of compounds S-141, S-140, S-143, and S-148. In some embodiments, composition S-183 includes about 35% to about 45% of compound S-141, about 20% to about 30% of compound S-140, about 20% to about 30% compound S-143, and about 5% to about 15% of compound S-148. In some embodiments, composition 183 includes about 40% of compound S-141, about 25% of compound S-140, about 25% compound S-143, and about 10% of compound S-148.

In some embodiments, the structural lipid is a pytosterol. In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

Ratio of Compounds

A lipid nanoparticle of the invention can include a structural component as described herein. The structural component of the lipid nanoparticle can be any one of compounds S-1-148, a mixture of one or more structural compounds of the invention and/or any one of compounds 5-1-148 combined with a cholesterol and/or a phytosterol.

For example, the structural component of the lipid nanoparticle can be a mixture of one or more structural compounds (e.g. any of Compounds 5-1-148) of the invention with cholesterol. The mol % of the structural compound present in the lipid nanoparticle relative to cholesterol can be from 0-99 mol %. The mol % of the structural compound present in the lipid nanoparticle relative to cholesterol can be about 10 mol %, 20 mol %, 30 mol %, 40 mol %, 50 mol %, 60 mol %, 70 mol %, 80 mol %, or 90 mol %.

In one aspect, the invention features a composition including two or more sterols, wherein the two or more sterols include at least two of: 3-sitosterol, sitostanol, camesterol, stigmasterol, and brassicasteol. The composition may additionally comprise cholesterol. In one embodiment, β-sitosterol comprises about 35-99%, e.g., about 40%, 50%, 60%, 70%, 80%, 90%, 95% or greater of the non-cholesterol sterol in the composition.

In another aspect, the invention features a composition including two or more sterols, wherein the two or more sterols include β-sitosterol and campesterol, wherein β-sitosterol includes 95-99.9% of the sterols in the composition and campesterol includes 0.1-5% of the sterols in the composition.

In some embodiments, the composition further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including two or more sterols, wherein the two or more sterols include β-sitosterol and sitostanol, wherein β-sitosterol includes 95-99.9% of the sterols in the composition and sitostanol includes 0.1-5% of the sterols in the composition.

In some embodiments, the composition further includes campesterol. In some embodiments, β-sitosterol includes 95-99.9%, campesterol includes 0.05-4.95%, and sitostanol includes 0.05-4.95% of the sterols in the composition.

In some embodiments, the composition further includes campesterol. In some embodiments, β-sitosterol includes 75-80%, campesterol includes 5-10%, and sitostanol includes 10-15% of the sterols in the composition.

In some embodiments, the composition further includes an additional sterol. In some embodiments, β-sitosterol includes 35-45%, stigmasterol includes 20-30%, and campesterol includes 20-30%, and brassicasterol includes 1-5% of the sterols in the composition.

In another aspect, the invention features a composition including a plurality of lipid nanoparticles, wherein the plurality of lipid nanoparticles include an ionizable lipid and two or more sterols, wherein the two or more sterols include 0-sitosterol, and campesterol and 3-sitosterol includes 95-99.9% of the sterols in the composition and campesterol includes 0.1-5% of the sterols in the composition.

In some embodiments, the two or more sterols further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including a plurality of lipid nanoparticles, wherein the plurality of lipid nanoparticles include an ionizable lipid and two or more sterols, wherein the two or more sterols include 0-sitosterol, and sitostanol and β-sitosterol includes 95-99.9% of the sterols in the composition and sitostanol includes 0.1-5% of the sterols in the composition.

In some embodiments, the two or more sterols further includes campesterol. In some embodiments, β-sitosterol includes 95-99.9%, campesterol includes 0.05-4.95%, and sitostanol includes 0.05-4.95% of the sterols in the composition.

(iii) Non-Cationic Helper Lipids/Phospholipids

In some embodiments, the lipid-based composition (e.g., LNP) described herein comprises one or more non-cationic helper lipids. In some embodiments, the non-cationic helper lipid is a phospholipid. In some embodiments, the non-cationic helper lipid is a phospholipid substitute or replacement.

As used herein, the term “non-cationic helper lipid” refers to a lipid comprising at least one fatty acid chain of at least 8 carbons in length and at least one polar head group moiety. In one embodiment, the helper lipid is not a phosphatidyl choline (PC). In one embodiment the non-cationic helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-i77-glycero-3-phosphocholine (DSPC) substitute.

Phospholipids

The lipid composition of the pharmaceutical composition disclosed herein can comprise one or more non-cationic helper lipids. In some embodiments, the non-cationic helper lipids are phospholipids, for example, one or more saturated or (poly)unsaturated phospholipids or a combination thereof. In general, phospholipids comprise a phospholipid moiety and one or more fatty acid moieties. As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

The lipid component of a lipid nanoparticle of the disclosure may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to Formula (H III):

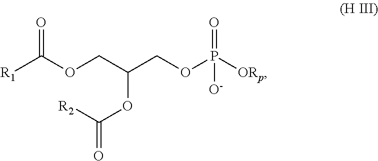

in which Rₚ represents a phospholipid moiety and R₁ and R₂ represent fatty acid moieties with or without unsaturation that may be the same or different. A phospholipid moiety may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a LNP to facilitate membrane permeation or cellular recognition or in conjugating a LNP to a useful component such as a targeting or imaging moiety (e.g., a dye). Each possibility represents a separate embodiment of the present invention.

Phospholipids useful in the compositions and methods described herein may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine (18:3 (cis) PC), 1,2-diarachidonoyl-sn-glycero-3-phosphocholine (DAPC), 1,2-didocosahexaenoyl-sn-glycero-3-phosphocholine (22:6 (cis) PC) 1,2-diphytanoyl-sn-glycero-3-phosphoethanolamine (4ME 16.0 PE), 1,2-distearoyl-sn-glycero-3-phosphoethanolamine (DSPE), 1,2-dilinoleoyl-sn-glycero-3-phosphoethanolamine (PE (18:2/18:2), 1,2-dilinolenoyl-sn-glycero-3-phosphoethanolamine (PE 18:3(9Z,12Z, 15Z), 1,2-diarachidonoyl-sn-glycero-3-phosphoethanolamine (DAPE 18:3 (9Z,12Z, 15Z), 1,2-didocosahexaenoyl-sn-glycero-3-phosphoethanolamine (22:6 (cis) PE), 1,2-dioleoyl-sn-glycero-3-phospho-rac-(1-glycerol) sodium salt (DOPG), and sphingomyelin. Each possibility represents a separate embodiment of the invention.

In some embodiments, a LNP includes DSPC. In certain embodiments, a LNP includes DOPE. In some embodiments, a LNP includes DMPE. In some embodiments, a LNP includes both DSPC and DOPE.

In one embodiment, a non-cationic helper lipid for use in an immune cell delivery LNP is selected from the group consisting of: DSPC, DMPE, and DOPC or combinations thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

Examples of phospholipids include, but are not limited to, the following:

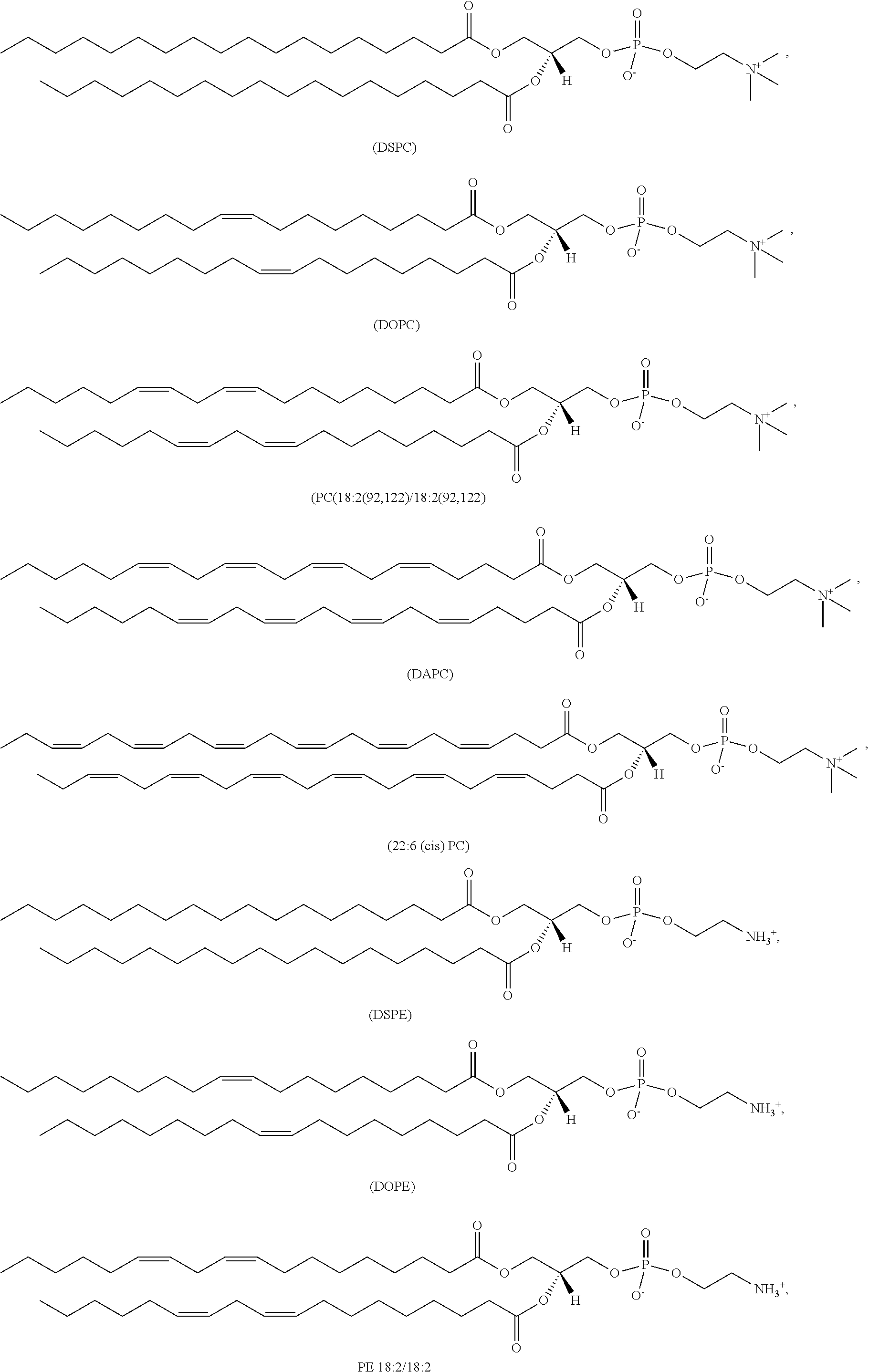

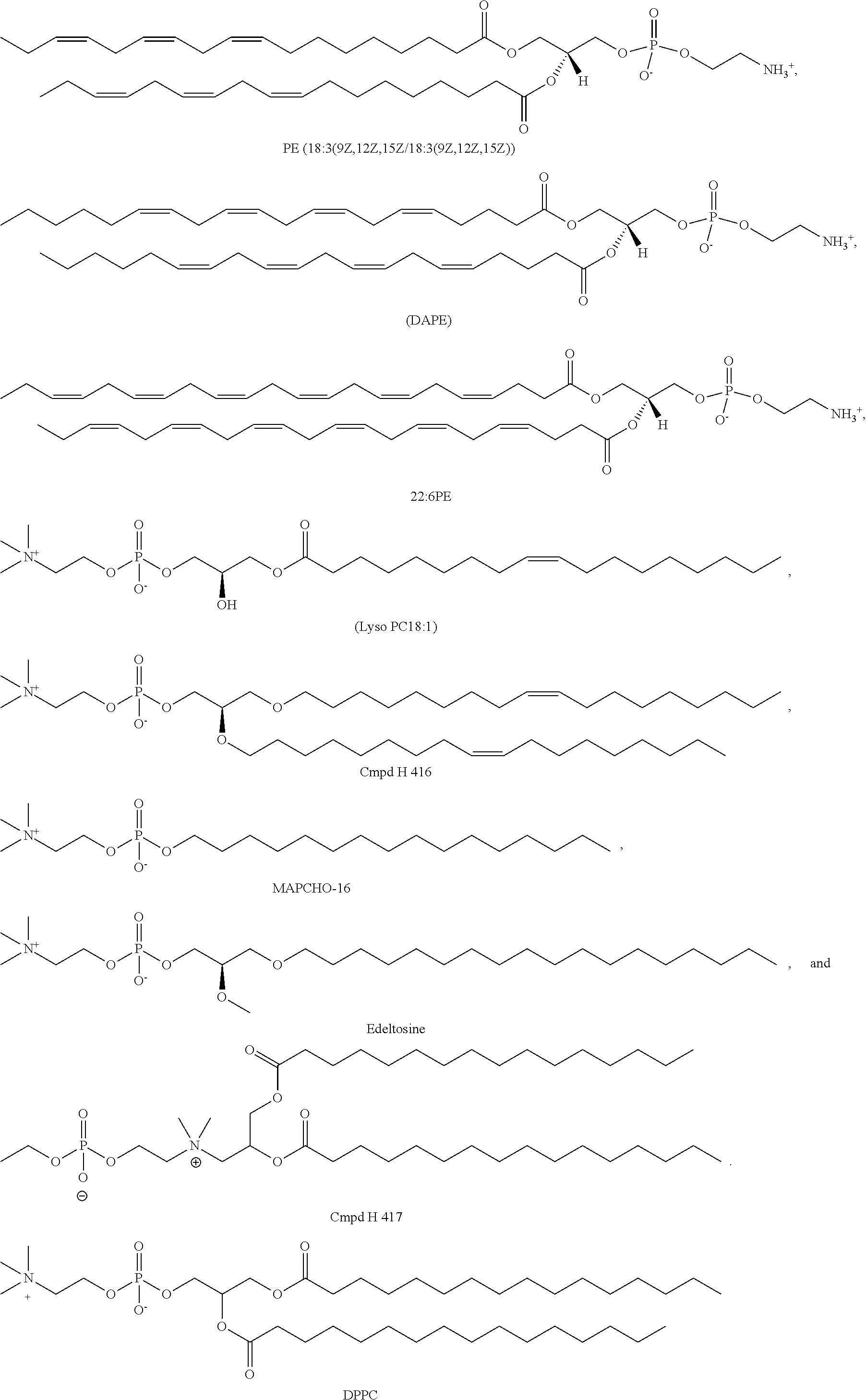

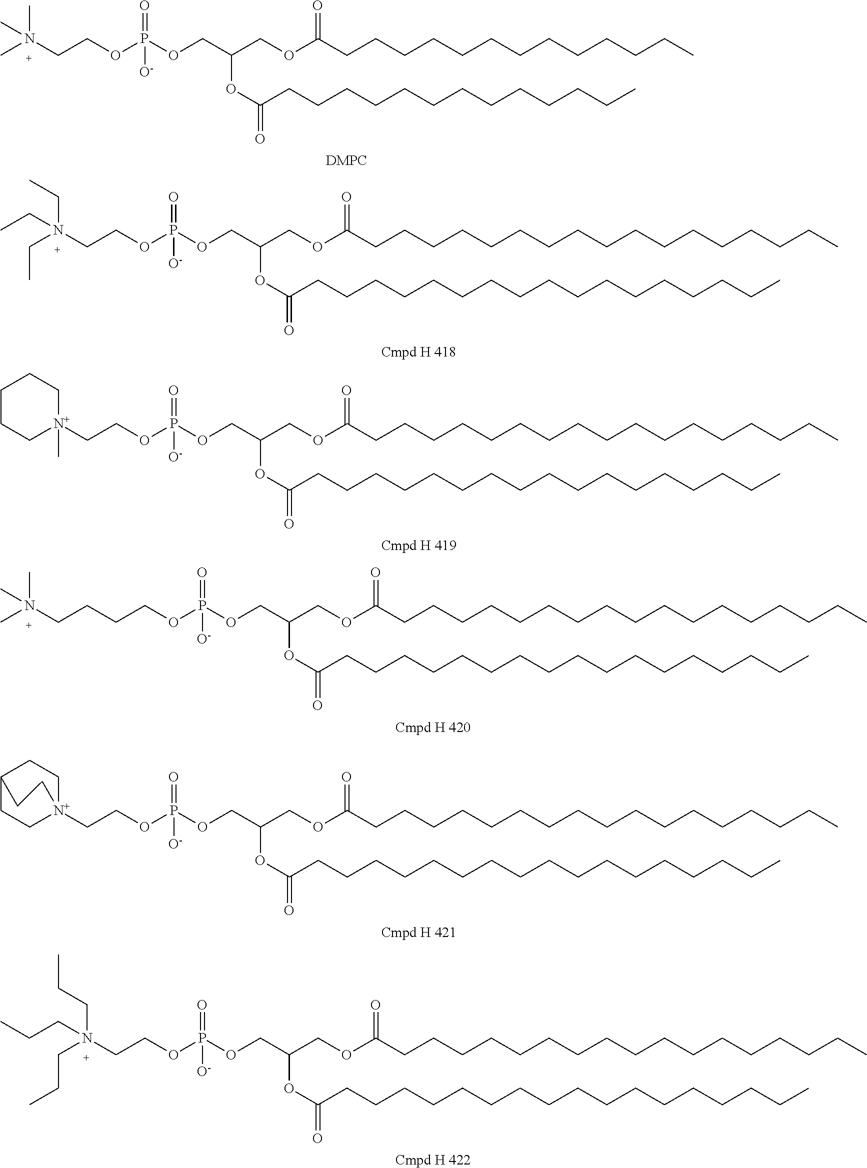

In certain embodiments, a phospholipid useful or potentially useful in the present invention is an analog or variant of DSPC (1,2-dioctadecanoyl-sn-glycero-3-phosphocholine). In certain embodiments, a phospholipid useful or potentially useful in the present invention is a compound of Formula (H IX):

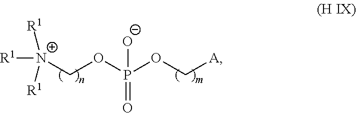

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

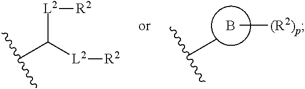

each instance of L² is independently a bond or optionally substituted C₁₋₆ alkylene, wherein one methylene unit of the optionally substituted C₁₋₆ alkylene is optionally replaced with

—O—, —N(R^{N})—, —S—, —C(O)—, —C(O)N(R^{N})—, —NR^{N}C(O)—, —C(O)O—, —O(O)—, —OC(O)O—, —OC(O)N(R^{N})—, —NR^{N}C(O)O—, or —NR^{N}C(O)N(R^{N})—;

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

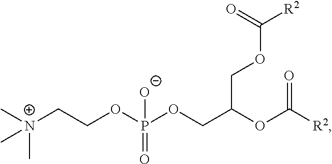

wherein each instance of R² is independently unsubstituted alkyl, unsubstituted alkenyl, or unsubstituted alkynyl.

i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R¹ is not methyl. In certain embodiments, at least one of R¹ is not hydrogen or methyl. In certain embodiments, the compound of Formula (IX) is of one of the following formulae:

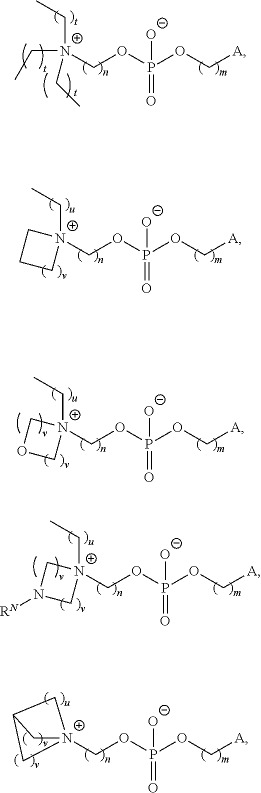

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, the compound of Formula (H IX) is of one of the following formulae:

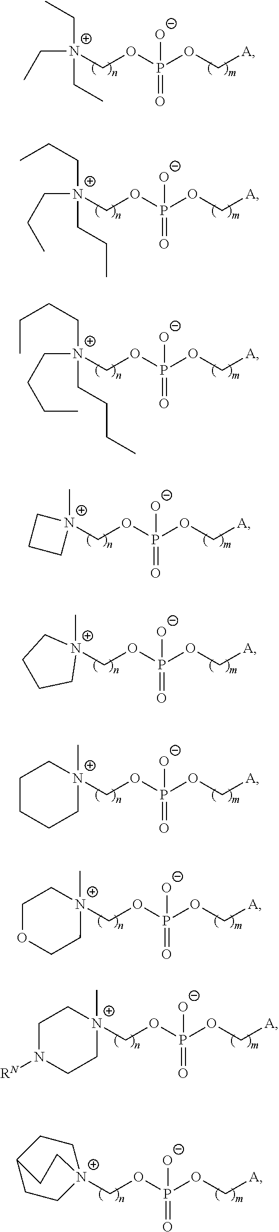

or a salt thereof.

In certain embodiments, a compound of Formula (H IX) is one of the following:

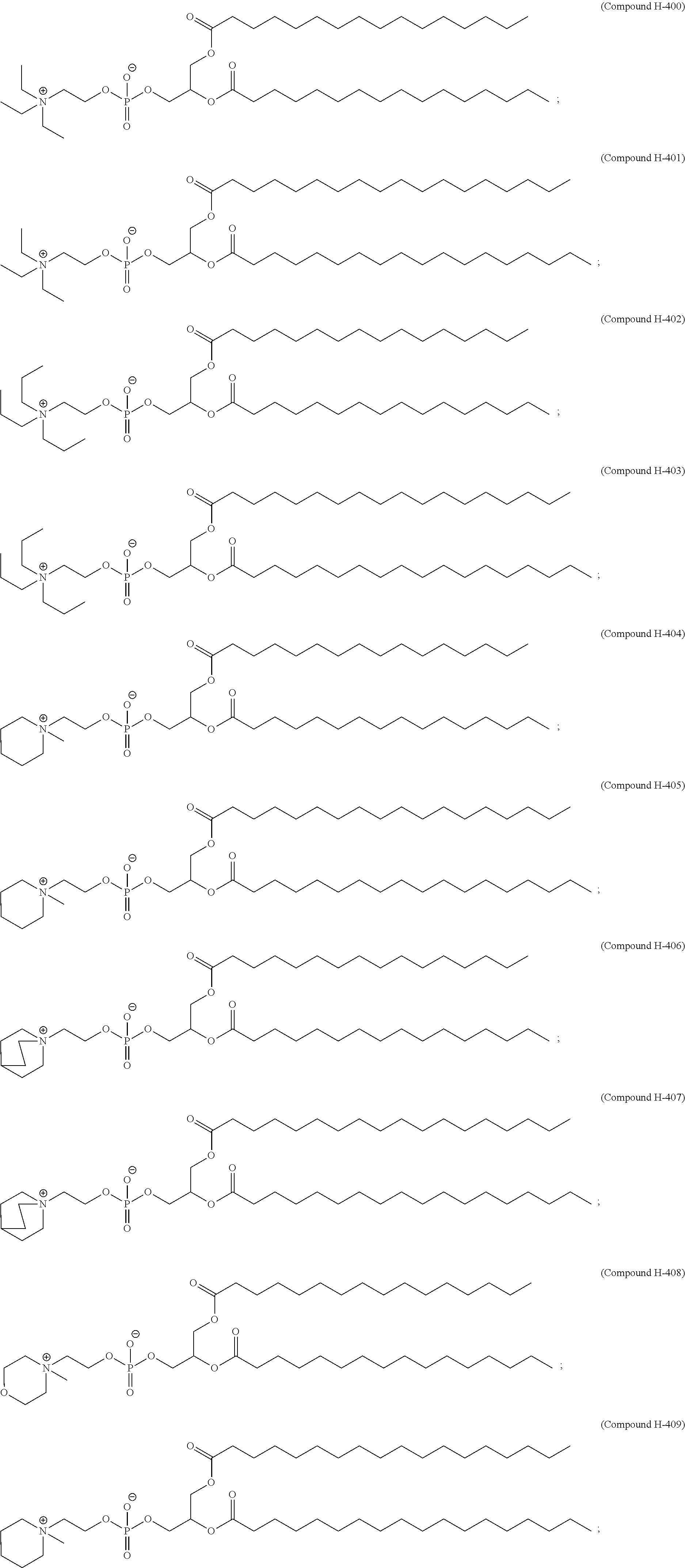

or a salt thereof.

In one embodiment, an immune cell delivery LNP comprises Compound H-409 as a non-cationic helper lipid.

### (ii) Phospholipid Tail Modifications

In certain embodiments, a phospholipid useful or potentially useful in the present invention comprises a modified tail. In certain embodiments, a phospholipid useful or potentially useful in the present invention is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H IX) is of Formula (H IX-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;

In certain embodiments, the compound of Formula (H IX) is of Formula (H IX-c):

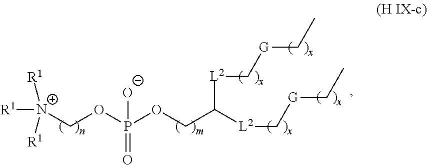

or a salt thereof, wherein:
each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H IX-c) is of Formula (H IX-c-1):

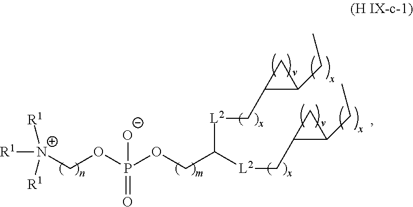

or salt thereof, wherein:
each instance of v is independently 1, 2, or 3.

In certain embodiments, the compound of Formula (H IX-c) is of Formula (H IX-c-2):

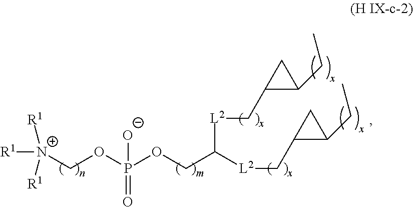

or a salt thereof.

In certain embodiments, the compound of Formula (IX-c) is of the following formula:

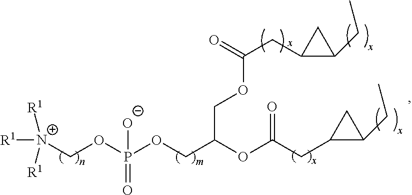

or a salt thereof.

In certain embodiments, the compound of Formula (H IX-c) is the following:

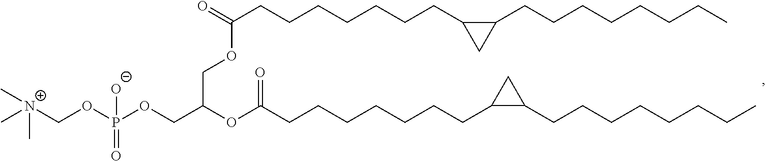 [SMILES: CCCCCCCCC1CC1CCCCCCCC(=O)OCC(COP(=O)([O-])OC[N+](C)(C)C)OC(=O)CCCCCCCC1CC1CCCCCCCC]

or a salt thereof.

In certain embodiments, the compound of Formula (H IX-c) is of Formula (H IX-c-3):

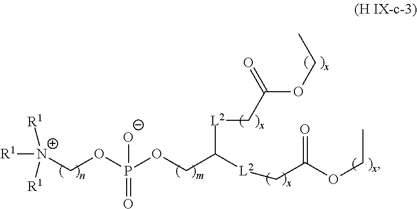

or a salt thereof.

In certain embodiments, the compound of Formula (H IX-c) is of the following formulae:

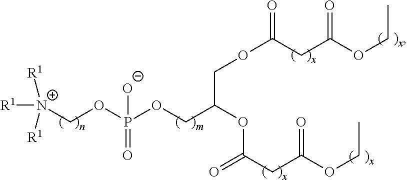

or a salt thereof.

In certain embodiments, the compound of Formula (H IX-c) is the following:

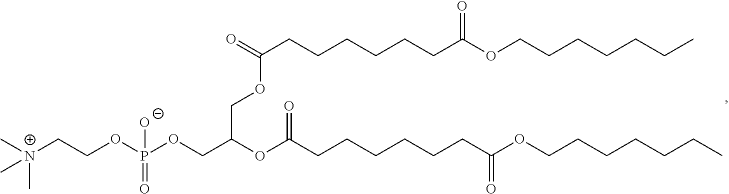 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (H IX), wherein n is 1, 3, 4, 5, 6, 7, 8, 9, or 10. For example, in certain embodiments, a compound of Formula (H IX) is of one of the following formulae:

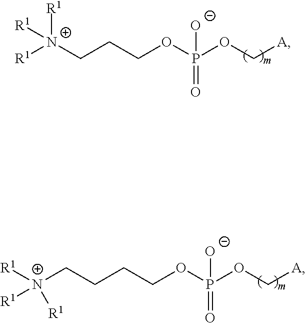

or a salt thereof.

In certain embodiments, a compound of Formula (H IX) is one of the following:

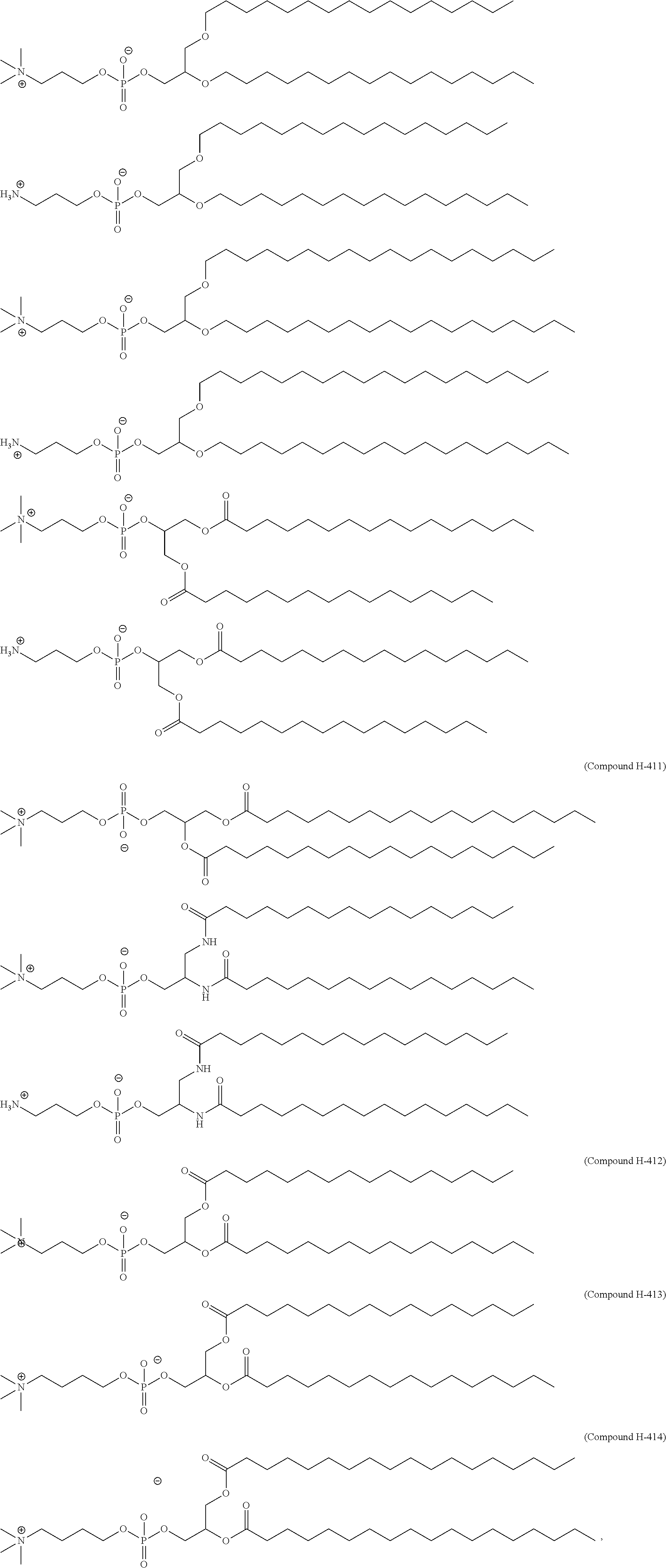

or salts thereof.

In certain embodiments, an alternative lipid is used in place of a phospholipid of the invention. Non-limiting examples of such alternative lipids include the following:

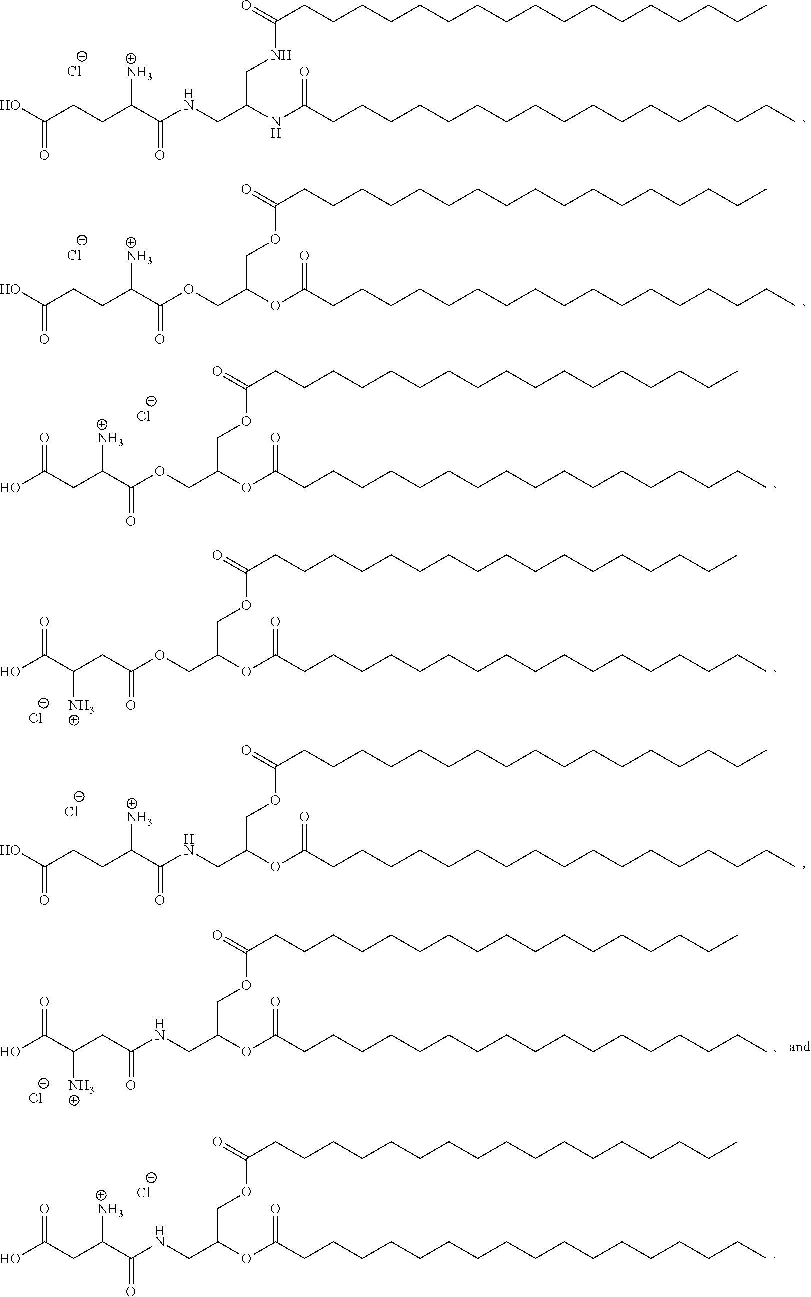

Phospholipid Tail Modifications

In certain embodiments, a phospholipid useful in the present invention comprises a modified tail. In certain embodiments, a phospholipid useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (H I) is of Formula (H I-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;

In certain embodiments, the compound of Formula (H I-a) is of Formula (H I-c):

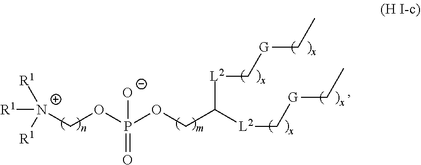

or a salt thereof, wherein:

each x is independently an integer between 0-30, inclusive; and

each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, S(O) OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In certain embodiments, the compound of Formula (H I-c) is of Formula (H I-c-1):

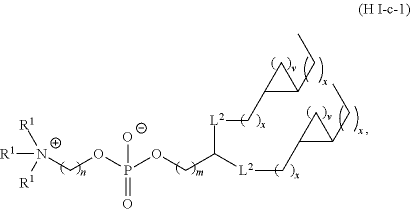

or salt thereof, wherein:

each instance of v is independently 1, 2, or 3.

In certain embodiments, the compound of Formula (H I-c) is of Formula (H I-c-2):

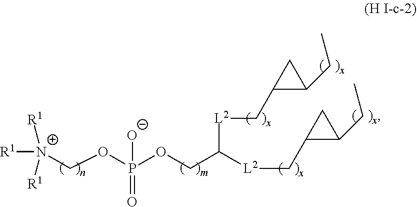

or a salt thereof.

In certain embodiments, the compound of Formula (I-c) is of the following formula:

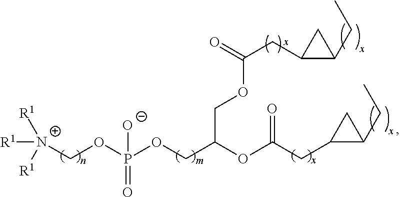

or a salt thereof.

In certain embodiments, the compound of Formula (H I-c) is the following:

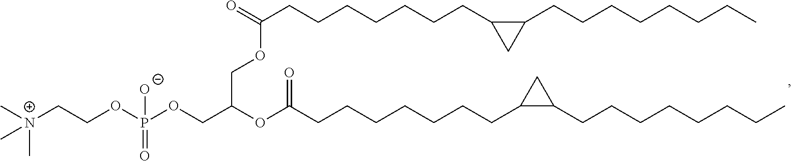 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (H I-c) is of Formula (H I-c-3):

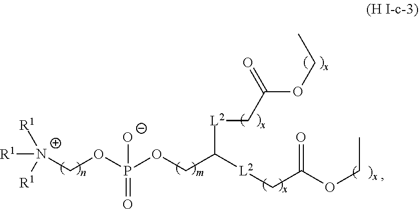

or a salt thereof.

In certain embodiments, the compound of Formula (H I-c) is of the following formulae:

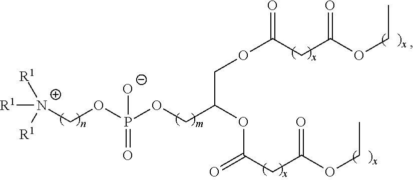

or a salt thereof.

In certain embodiments, the compound of Formula (H I-c) is the following:

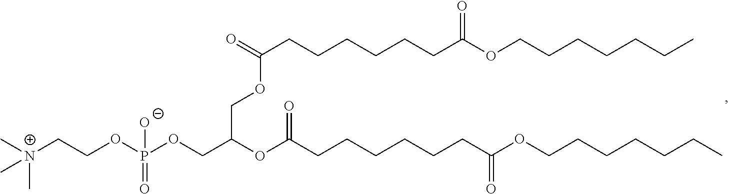 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

Phosphocholine Linker Modifications

In certain embodiments, a phospholipid useful in the present invention comprises a modified phosphocholine moiety, wherein the alkyl chain linking the quaternary amine to the phosphoryl group is not ethylene (e.g., n is not 2). Therefore, in certain embodiments, a phospholipid useful in the present invention is a compound of Formula (H I), wherein n is 1, 3, 4, 5, 6, 7, 8, 9, or 10. For example, in certain embodiments, a compound of Formula (H I) is of one of the following formulae:

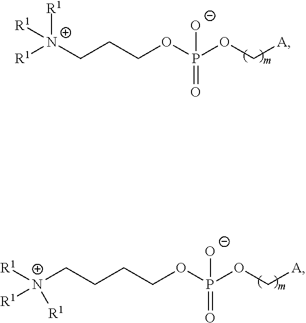

or a salt thereof.

In certain embodiments, a compound of Formula (H I) is one of the following:

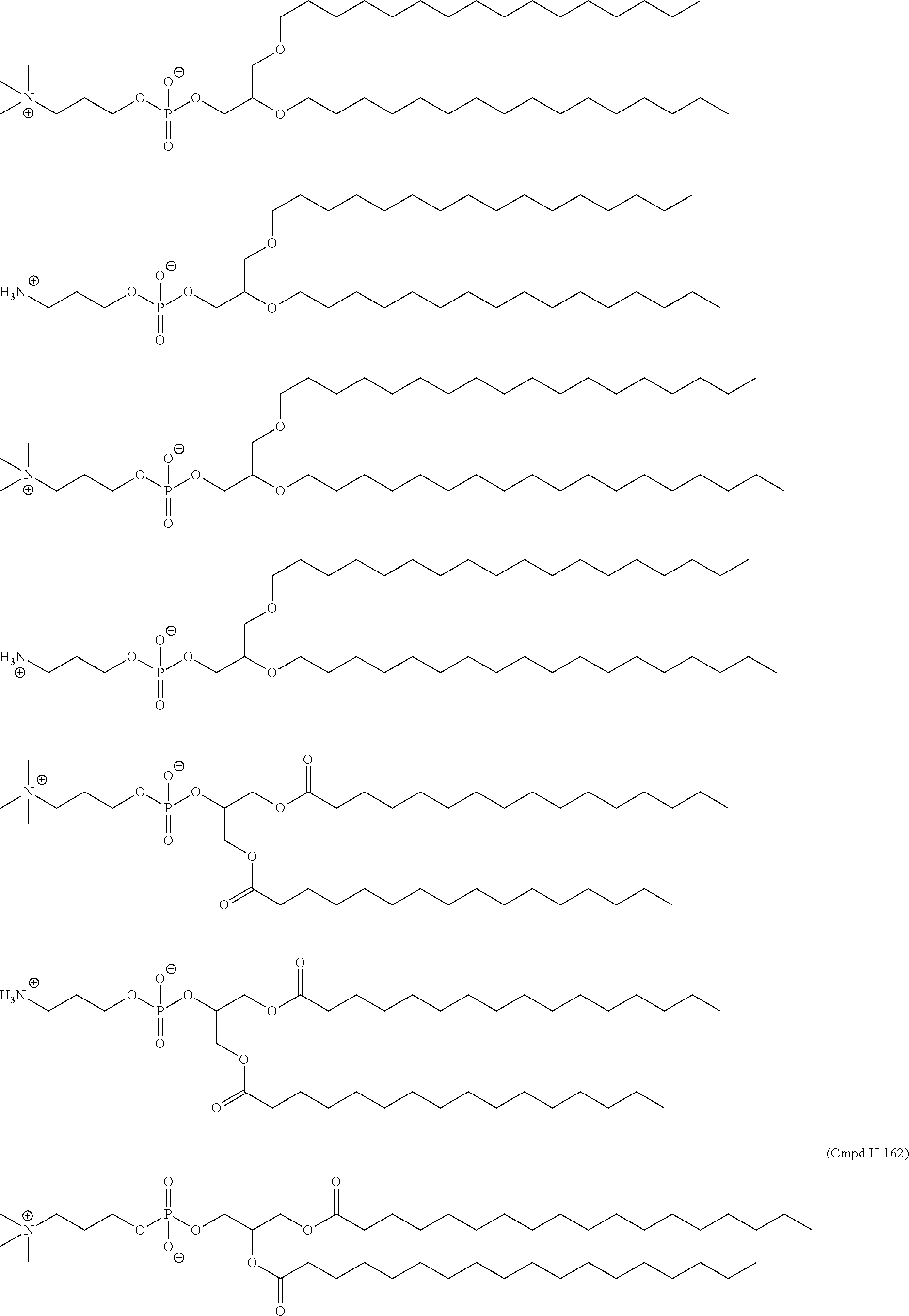

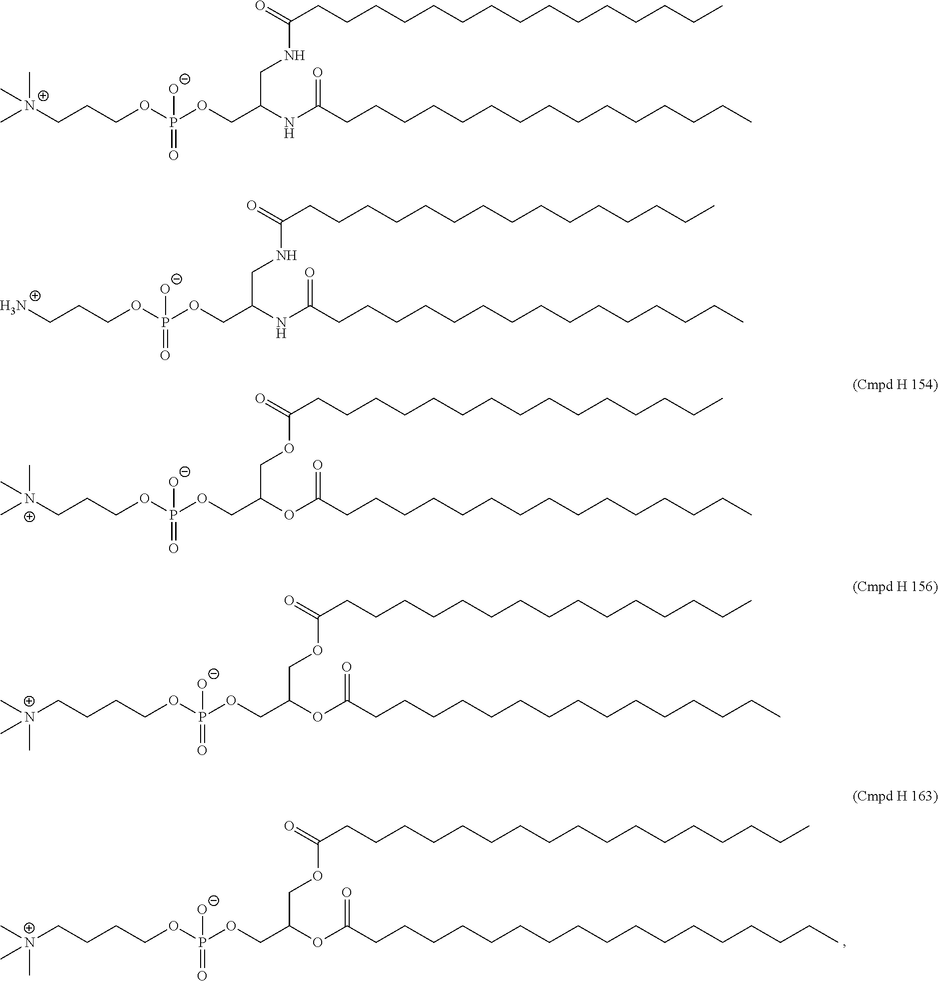

or salts thereof.

Numerous LNP formulations having phospholipids other than DSPC were prepared and tested for activity, as demonstrated in the examples below.

Phospholipid Substitute or Replacement

In some embodiments, the lipid-based composition (e.g., lipid nanoparticle) comprises an oleic acid or an oleic acid analog in place of a phospholipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the lipid-based composition (e.g., lipid nanoparticle) comprises a different zwitterionic group in place of a phospholipid.

Exemplary phospholipid substitutes and/or replacements are provided in Published PCT Application WO 2017/099823, herein incorporated by reference.

Exemplary phospholipid substitutes and/or replacements are provided in Published PCT Application WO 2017/099823, herein incorporated by reference.

(iv) PEG Lipids

Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

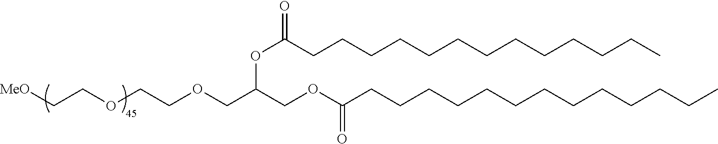

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In some embodiments, the PEG lipid is a compound of Formula (PI):

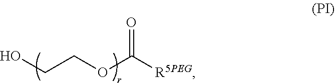

or a salt or isomer thereof, wherein:

r is an integer between 1 and 100;

R^{5PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

For example, R^{5PEG} is C₁₇ alkyl. For example, the PEG lipid is a compound of Formula (PI-a):

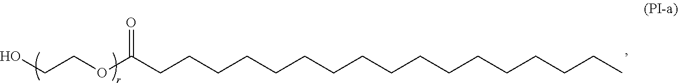

or a salt or isomer thereof, wherein r is an integer between 1 and 100.

For example, the PEG lipid is a compound of the following formula:

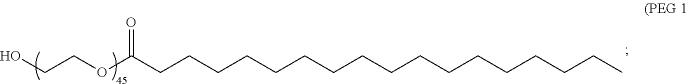

also referred to as Compound 428 below),
   or a salt or isomer thereof.

The PEG lipid may be a compound of Formula (PII):

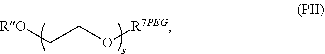

or a salt or isomer thereof, wherein:

s is an integer between 1 and 100;

R″ is a hydrogen, C₁₋₁₀ alkyl, or an oxygen protecting group;

R^{7PEG} is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group.

In some embodiments, R^{7PEG} is C₁₀₋₆₀ alkyl, and one or more of the methylene groups of R^{7PEG} are replaced with —C(O)—. For example, R^{7PEG} is C₃₁ alkyl, and two of the methylene groups of R^{7PEG} are replaced with —C(O)—.

In some embodiments, R″ is methyl.

In some embodiments, the PEG lipid is a compound of Formula (PII-a):

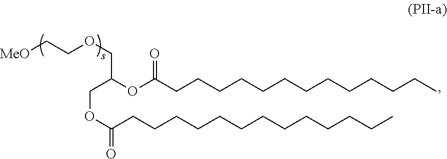

or a salt or isomer thereof, wherein s is an integer between 1 and 100.

For example, the PEG lipid is a compound of the following formula:

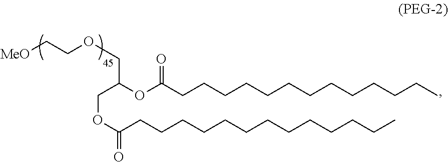

or a salt or isomer thereof.

In certain embodiments, a PEG lipid useful in the present invention is a compound of Formula (Pill). Provided herein are compounds of Formula (Pill):

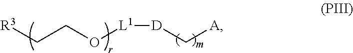

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

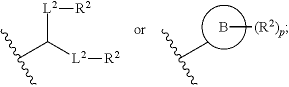

each instance of L² is independently a bond or optionally substituted C₁₋₆ alkylene, wherein one methylene unit of the optionally substituted C₁₋₆ alkylene is optionally replaced with 0, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O) S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (PIII) is a PEG-OH lipid (i.e., R³ is —OR^{O}, and R^{O} is hydrogen). In certain embodiments, the compound of Formula (PIII) is of Formula (PIII-OH):

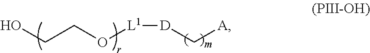

or a salt thereof.

In certain embodiments, D is a moiety obtained by click chemistry (e.g., triazole). In certain embodiments, the compound of Formula (PIII) is of Formula (PIII-a-1) or (PIII-a-2):

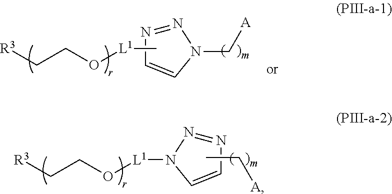

or a salt thereof.

In certain embodiments, the compound of Formula (PIII) is of one of the following formulae:

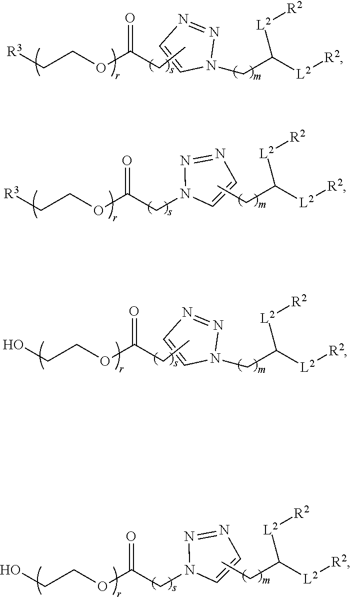

or a salt thereof, wherein

s is 0, 1, 2, 3, 4, 5, 6, 7, 8, 9, or 10.

In certain embodiments, the compound of Formula (PIII) is of one of the following formulae:

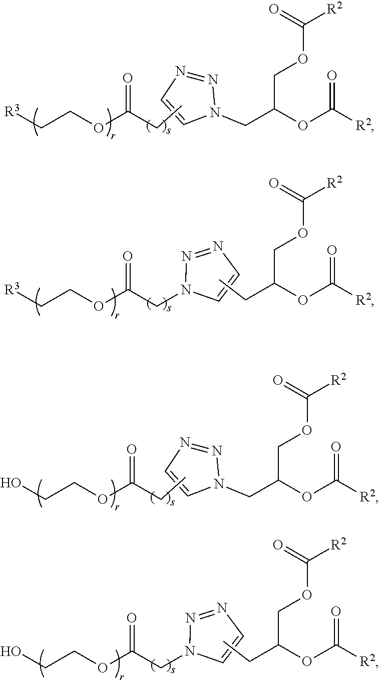

or a salt thereof.

In certain embodiments, a compound of Formula (PIII) is of one of the following formulae:

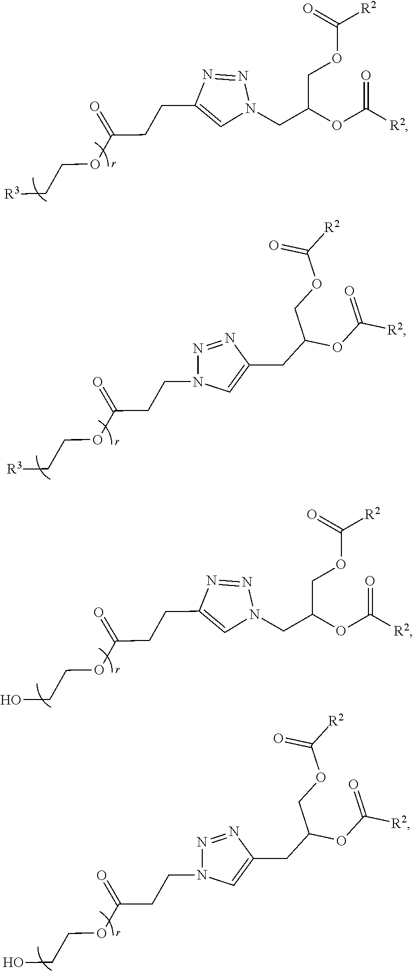

or a salt thereof.

In certain embodiments, a compound of Formula (PIII) is of one of the following formulae:

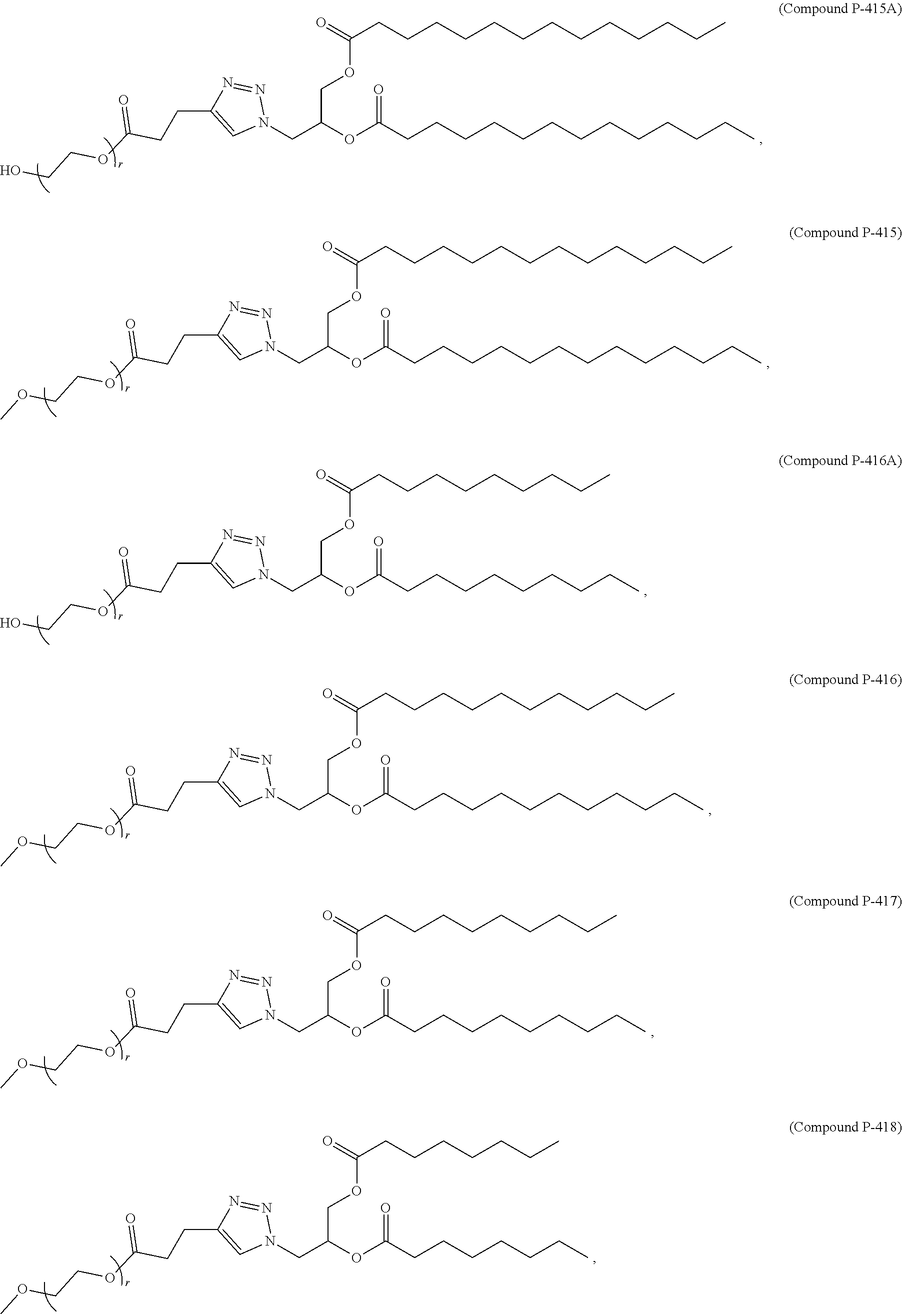

or a salt thereof.

In certain embodiments, D is a moiety cleavable under physiological conditions (e.g., ester, amide, carbonate, carbamate, urea). In certain embodiments, a compound of Formula (PIII) is of Formula (PIII-b-1) or (PIII-b-2):

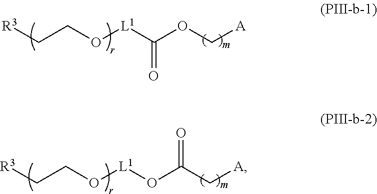

or a salt thereof.

In certain embodiments, a compound of Formula (PIII) is of Formula (PIII-b-1-OH) or (PIII-b-2-OH):

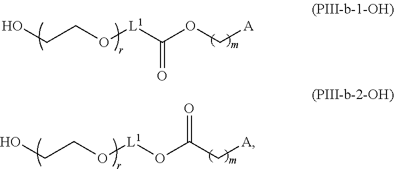

or a salt thereof.

In certain embodiments, the compound of Formula (PIII) is of one of the following formulae:

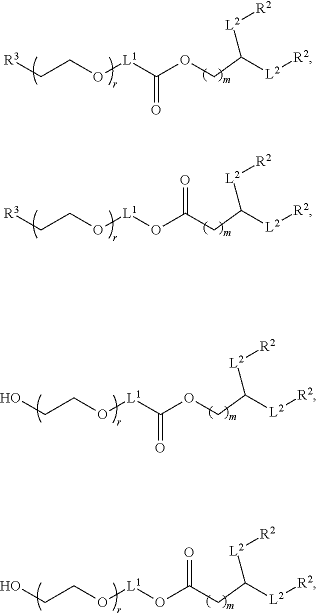

or a salt thereof.

In certain embodiments, a compound of Formula (PIII) is of one of the following formulae:

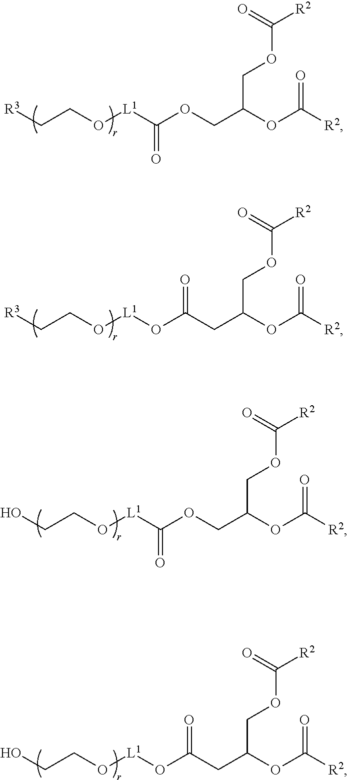

or a salt thereof.

In certain embodiments, a compound of Formula (PIII) is of one of the following formulae:

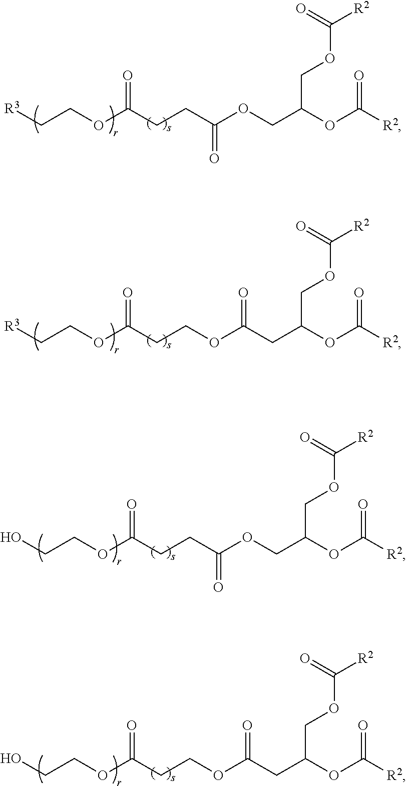

or a salt thereof.

In certain embodiments, a compound of Formula (PIII) is of one of the following formulae:

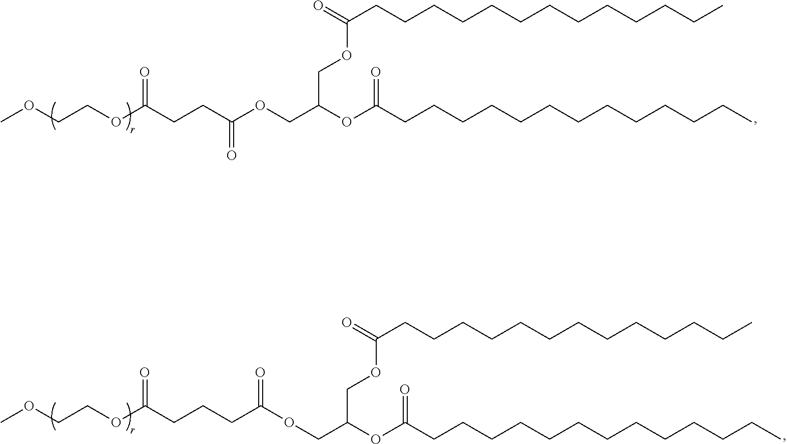

or salts thereof.

In certain embodiments, a PEG lipid useful in the present invention is a PEGylated fatty acid. In certain embodiments, a PEG lipid useful in the present invention is a compound of Formula (PIV). Provided herein are compounds of Formula (PIV):

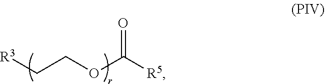

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (PIV is of Formula (PIV-OH):

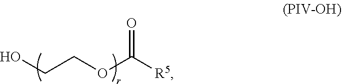

or a salt thereof. In some embodiments, r is 40-50. In some embodiments, r is 45.

In certain embodiments, a compound of Formula (PIV) is of one of the following formulae:

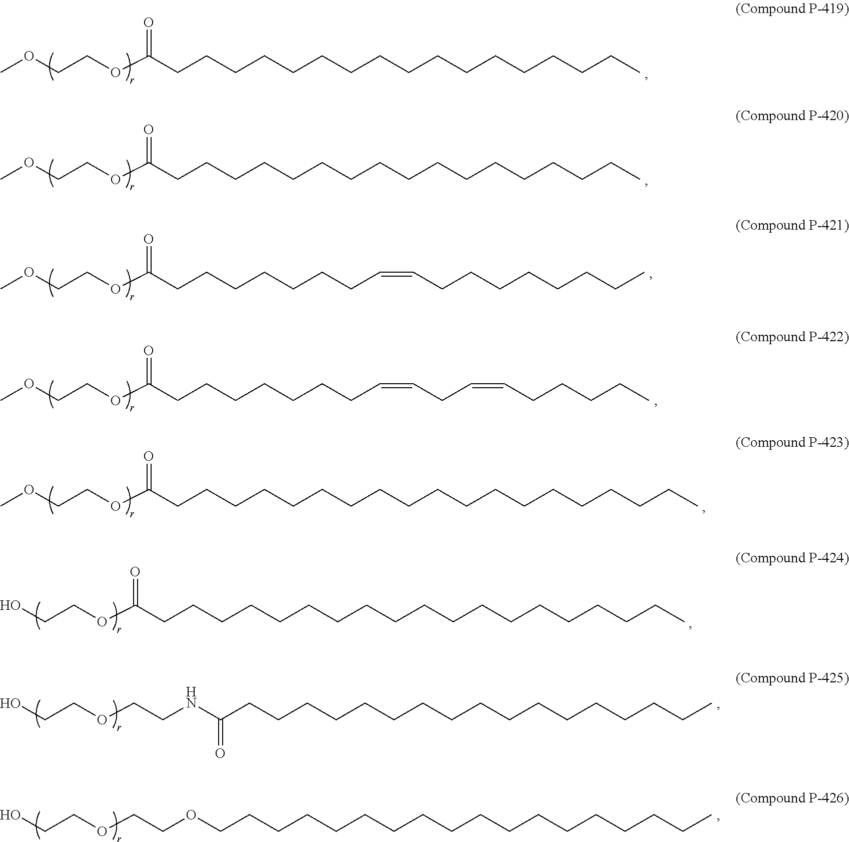

or a salt thereof. In some embodiments, r is 40-50. In some embodiments, r is 45.

In yet other embodiments the compound of Formula (PIV) is:

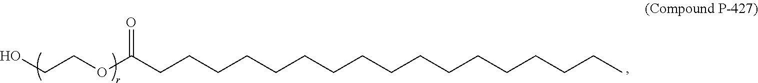

or a salt thereof.

In one embodiment, the compound of Formula (PIV) is

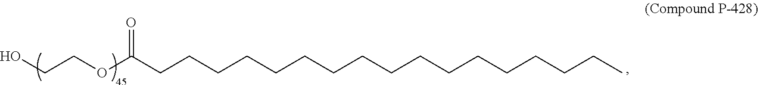

In one aspect, provided herein are lipid nanoparticles (LNPs) comprising PEG lipids of Formula (PV):

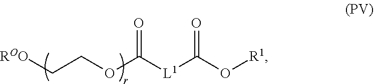

or pharmaceutically acceptable salts thereof; wherein:

L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;

R¹ is optionally substituted C₅₋₃₀ alkyl, optionally substituted C₅₋₃₀ alkenyl, or optionally substituted C₅₋₃₀ alkynyl;

R^{O} is hydrogen, optionally substituted alkyl, optionally substituted acyl, or an oxygen protecting group; and

r is an integer from 2 to 100, inclusive.

In certain embodiments, the PEG lipid of Formula (PV) is of the following formula:

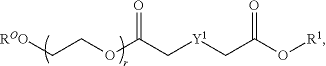

or a pharmaceutically acceptable salt thereof; wherein:

Y¹ is a bond, —CR₂—, —O—, —NR^{N}—, or —S—;

each instance of R is independently hydrogen, halogen, or optionally substituted alkyl; and

R^{N} is hydrogen, optionally substituted alkyl, optionally substituted acyl, or a nitrogen protecting group.

In certain embodiments, the PEG lipid of Formula (PV) is of one of the following formulae:

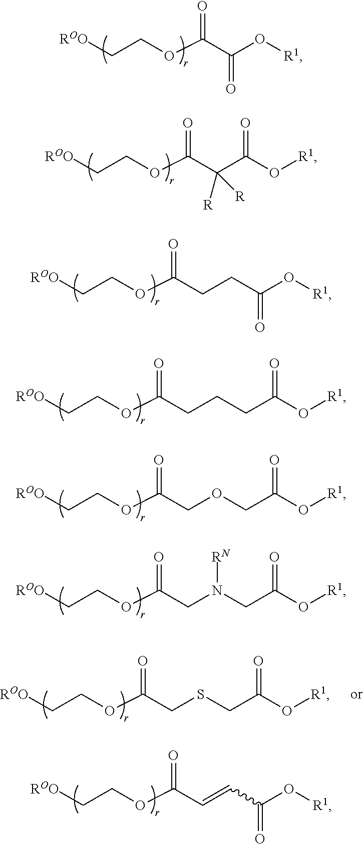

or a pharmaceutically acceptable salt thereof, wherein:

each instance of R is independently hydrogen, halogen, or optionally substituted alkyl.

In certain embodiments, the PEG lipid of Formula (PV) is of one of the following formulae:

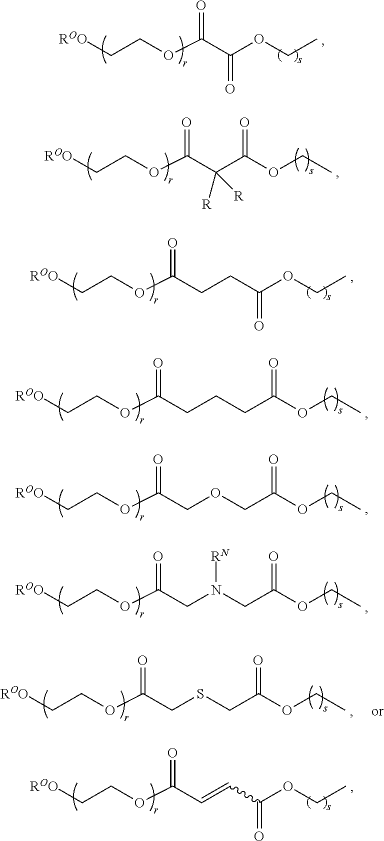

or a pharmaceutically acceptable salt thereof; wherein:

s is an integer from 5-25, inclusive.

In certain embodiments, the PEG lipid of Formula (PV) is of one of the following formulae:

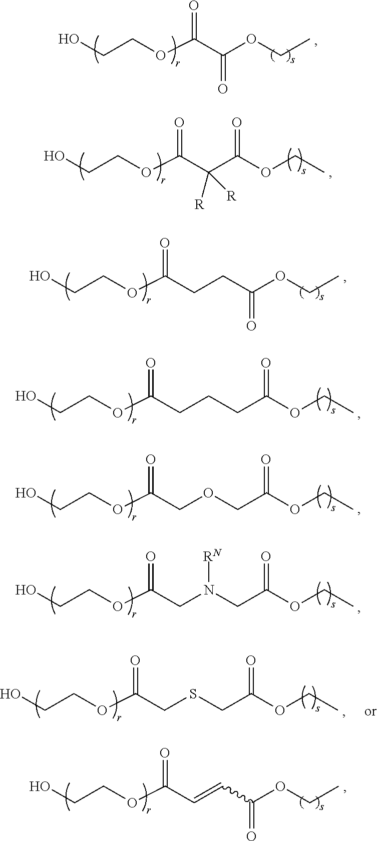

or a pharmaceutically acceptable salt thereof.

In certain embodiments, the PEG lipid of Formula (PV) is selected from the group consisting of:

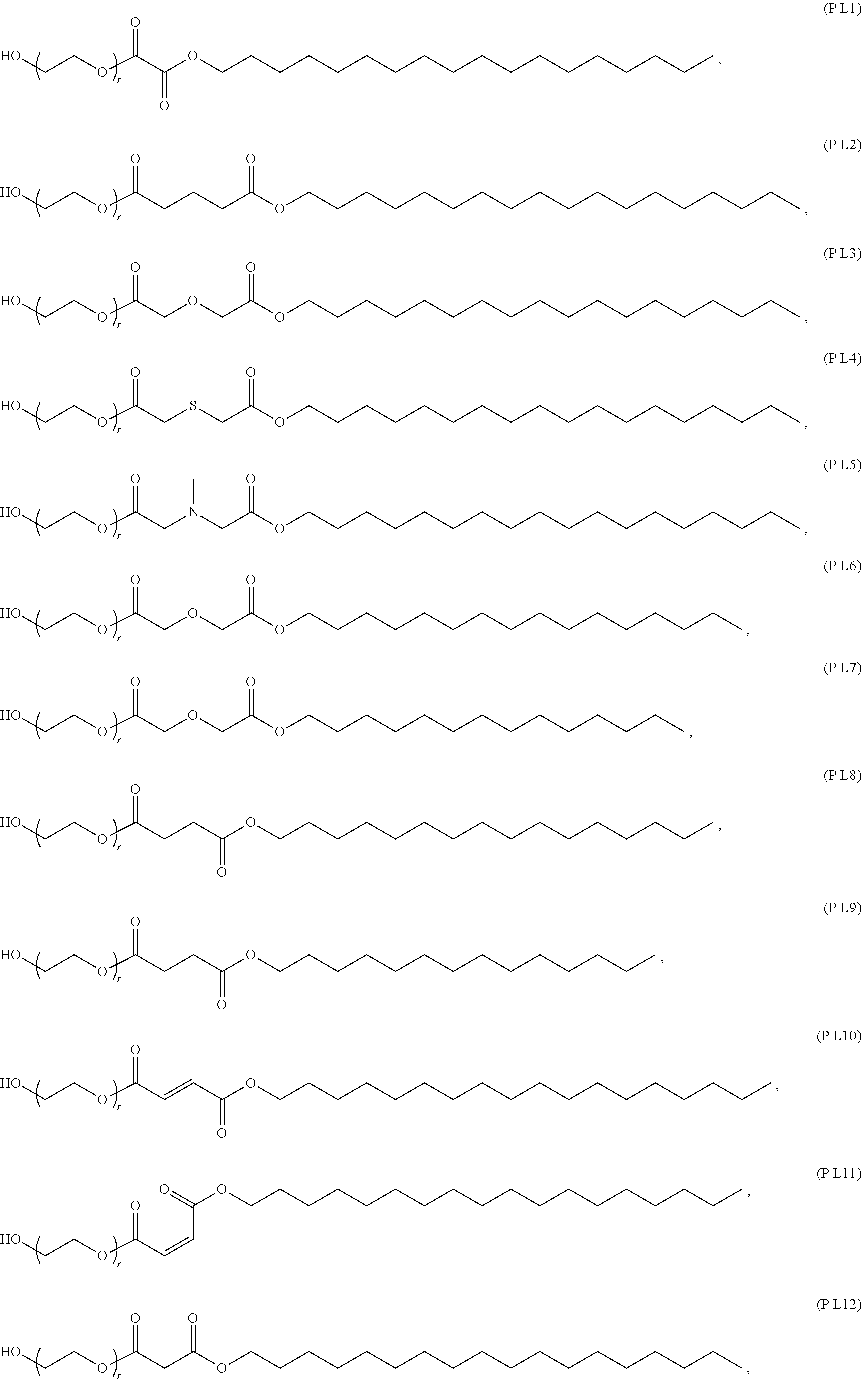

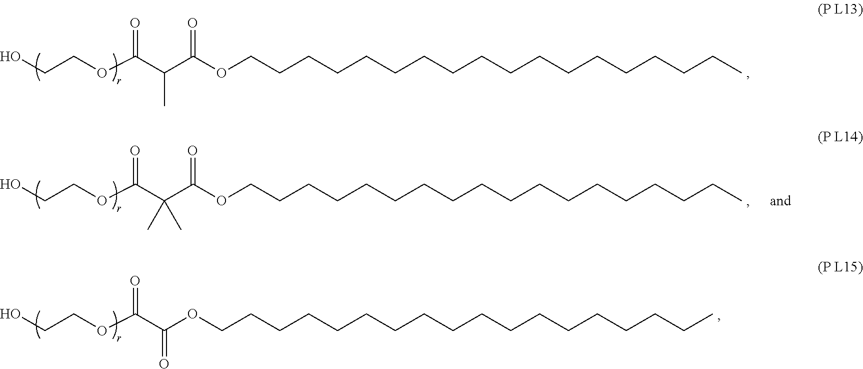

and pharmaceutically acceptable salts thereof.

In another aspect, provided herein are lipid nanoparticles (LNPs) comprising PEG lipids of Formula (PVI):

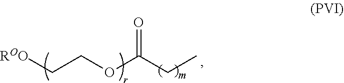

or pharmaceutically acceptable salts thereof; wherein:

R^{O} is hydrogen, optionally substituted alkyl, optionally substituted acyl, or an oxygen protecting group;

r is an integer from 2 to 100, inclusive; and

m is an integer from 5-15, inclusive, or an integer from 19-30, inclusive.

In certain embodiments, the PEG lipid of Formula (PVI) is of one of the following formulae:

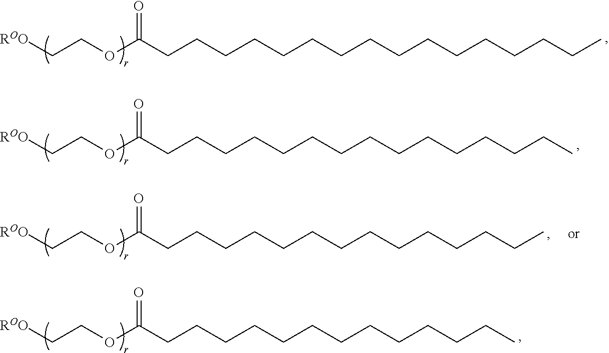

or a pharmaceutically acceptable salt thereof.

In certain embodiments, the PEG lipid of Formula (PVI) is of one of the following formulae:

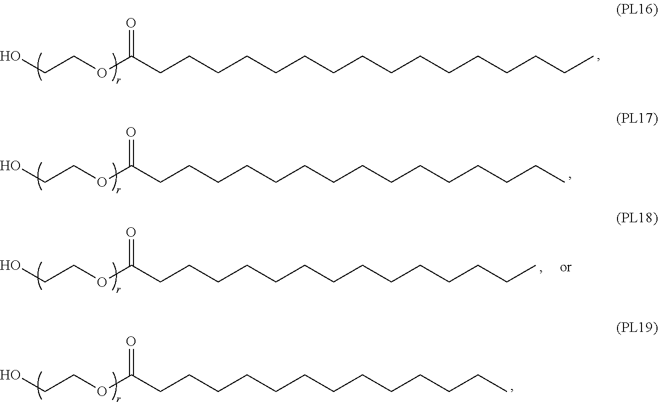

or a pharmaceutically acceptable salt thereof.

In another aspect, provided herein are lipid nanoparticles (LNPs) comprising PEG lipids of Formula (PVII):

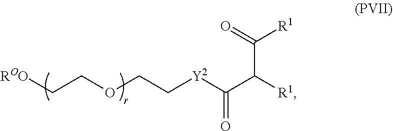

or pharmaceutically acceptable salts thereof, wherein:

Y² is —O—, —NR^{N}, or —S—

each instance of R¹ is independently optionally substituted C₅₋₃₀ alkyl, optionally substituted C₅₋₃₀ alkenyl, or optionally substituted C₅₋₃₀ alkynyl;

R^{O} is hydrogen, optionally substituted alkyl, optionally substituted acyl, or an oxygen protecting group;

R^{N} is hydrogen, optionally substituted alkyl, optionally substituted acyl, or a nitrogen protecting group; and

r is an integer from 2 to 100, inclusive.

In certain embodiments, the PEG lipid of Formula (PVII) is of one of the following formulae:

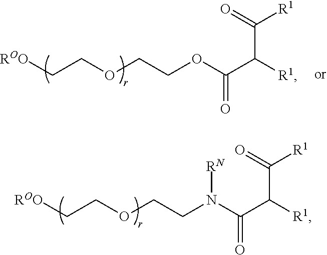

or a pharmaceutically acceptable salt thereof.

In certain embodiments, the PEG lipid of Formula (PVII) is of one of the following formulae:

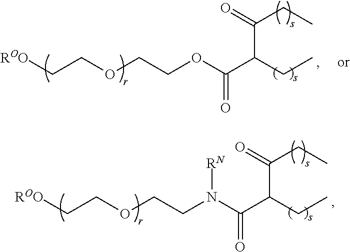

or a pharmaceutically acceptable salt thereof; wherein:

each instance of s is independently an integer from 5-25, inclusive.

In certain embodiments, the PEG lipid of Formula (PVII) is of one of the following formulae:

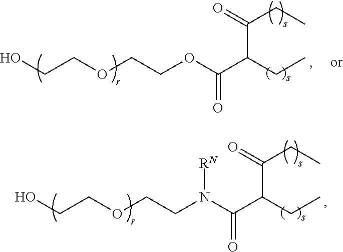

or a pharmaceutically acceptable salt thereof.

In certain embodiments, the PEG lipid of Formula (PVII) is selected from the group consisting of:

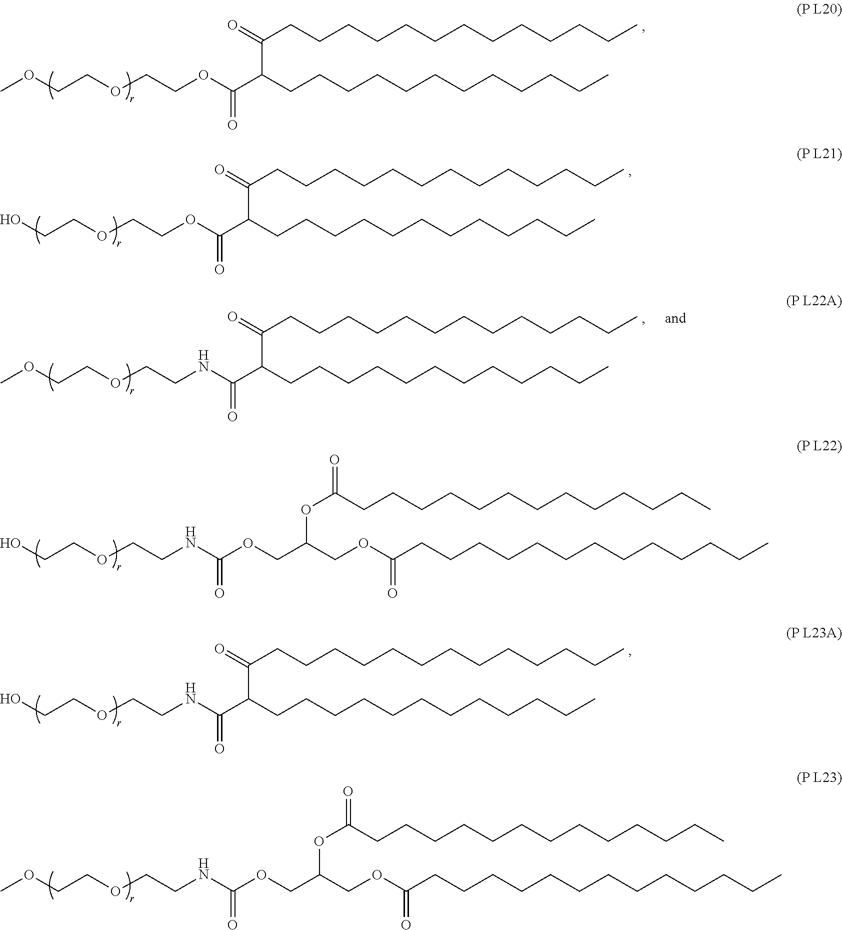

and pharmaceutically acceptable salts thereof.

In another aspect, provided herein are lipid nanoparticles (LNPs) comprising PEG lipids of Formula (PVIII):

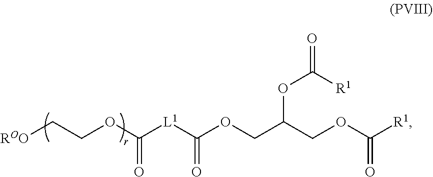

or pharmaceutically acceptable salts thereof, wherein:

L¹ is a bond, optionally substituted C₁₋₃ alkylene, optionally substituted C₁₋₃ heteroalkylene, optionally substituted C₂₋₃ alkenylene, optionally substituted C₂₋₃ alkynylene;

each instance of R¹ is independently optionally substituted C₅₋₃₀ alkyl, optionally substituted C₃₋₃₀ alkenyl, or optionally substituted C₅₋₃₀ alkynyl;

R^{O} is hydrogen, optionally substituted alkyl, optionally substituted acyl, or an oxygen protecting group;

r is an integer from 2 to 100, inclusive;

provided that when L¹ is —CH₂CH₂— or —CH₂CH₂CH₂—, R^{O} is not methyl.

In certain embodiments, when L¹ is optionally substituted C₂ or C₃ alkylene, R^{O} is not optionally substituted alkyl. In certain embodiments, when L¹ is optionally substituted C₂ or C₃ alkylene, R^{O} is hydrogen. In certain embodiments, when L¹ is —CH₂CH₂— or —CH₂CH₂CH₂—, R^{O} is not optionally substituted alkyl. In certain embodiments, when L¹ is —CH₂CH₂— or —CH₂CH₂CH₂—, R^{O} is hydrogen.

In certain embodiments, the PEG lipid of Formula (PVIII) is of the formula:

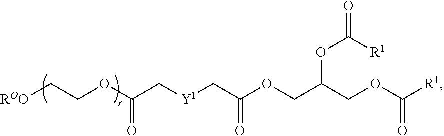

or a pharmaceutically acceptable salt thereof, wherein:

Y¹ is a bond, —CR₂—, —O—, —NR^{N}—, or —S—;

each instance of R is independently hydrogen, halogen, or optionally substituted alkyl;

R^{N} is hydrogen, optionally substituted alkyl, optionally substituted acyl, or a nitrogen protecting group;

provided that when Y¹ is a bond or —CH₂—, R^{O} is not methyl.

In certain embodiments, when L¹ is —CR₂—, R^{O} is not optionally substituted alkyl. In certain embodiments, when L¹ is —CR₂—, R^{O} is hydrogen. In certain embodiments, when L¹ is —CH₂—, R^{O} is not optionally substituted alkyl. In certain embodiments, when L¹ is —CH₂—, R^{O} is hydrogen.

In certain embodiments, the PEG lipid of Formula (PVIII) is of one of the following formulae:

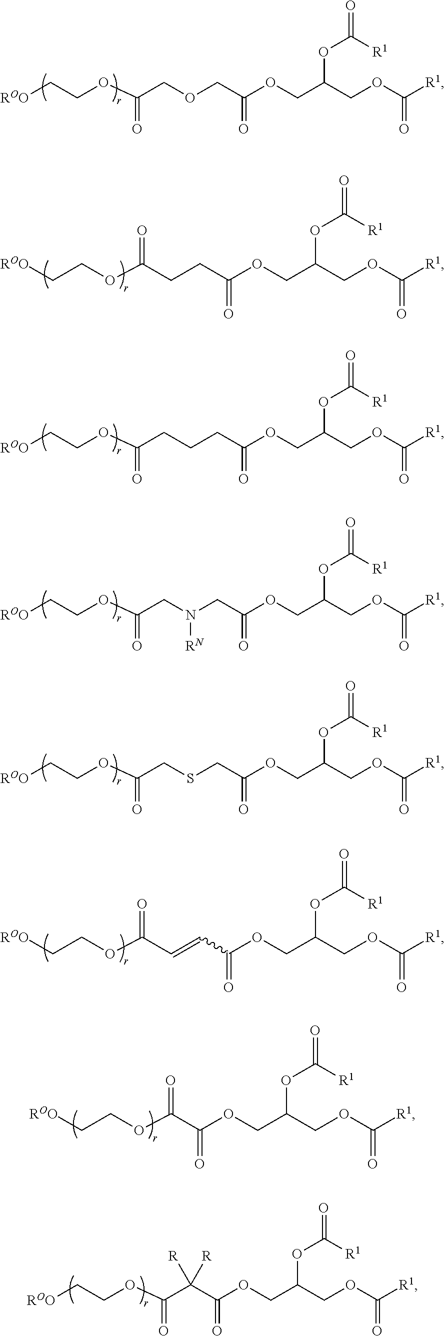

or a pharmaceutically acceptable salt thereof, wherein:

each instance of R is independently hydrogen, halogen, or optionally substituted alkyl.

In certain embodiments, the PEG lipid of Formula (PVIII) is of one of the following formulae:

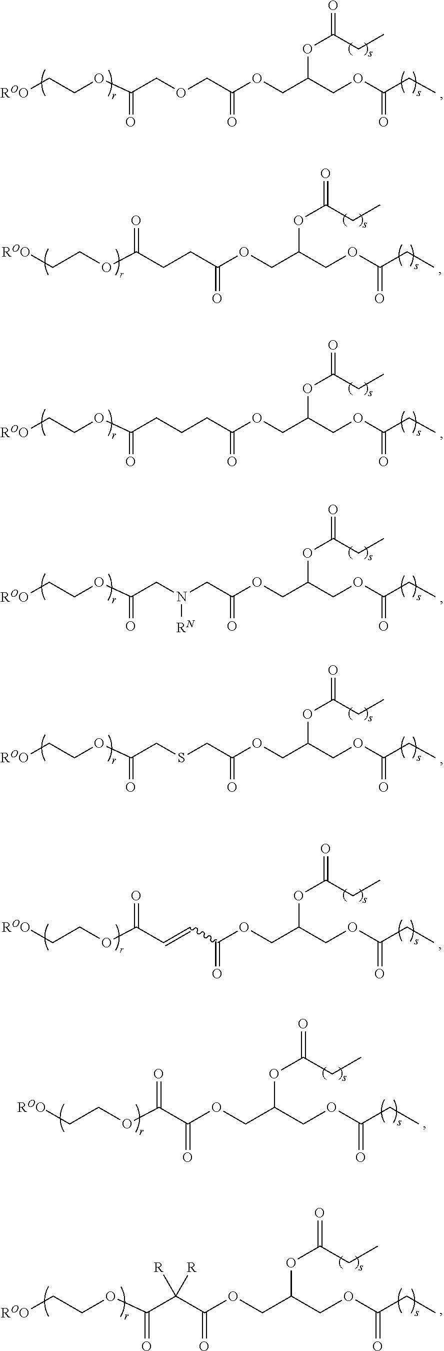

or a pharmaceutically acceptable salt thereof; wherein:

each instance of R is independently hydrogen, halogen, or optionally substituted alkyl; and

each s is independently an integer from 5-25, inclusive.

In certain embodiments, the PEG lipid of Formula (PVIII) is of one of the following formulae:

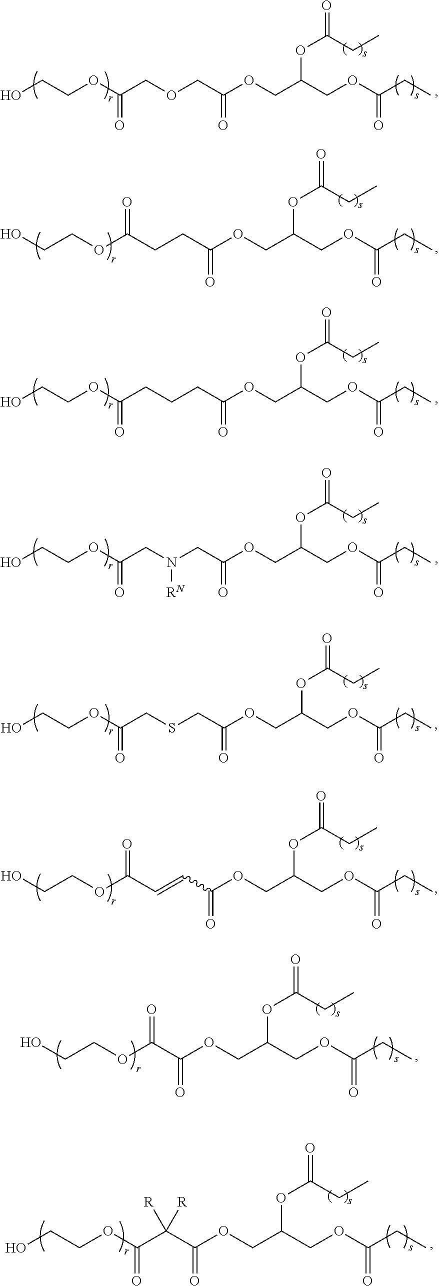

or a pharmaceutically acceptable salt thereof.

In certain embodiments, the PEG lipid of Formula (PVIII) is selected from the group consisting of:

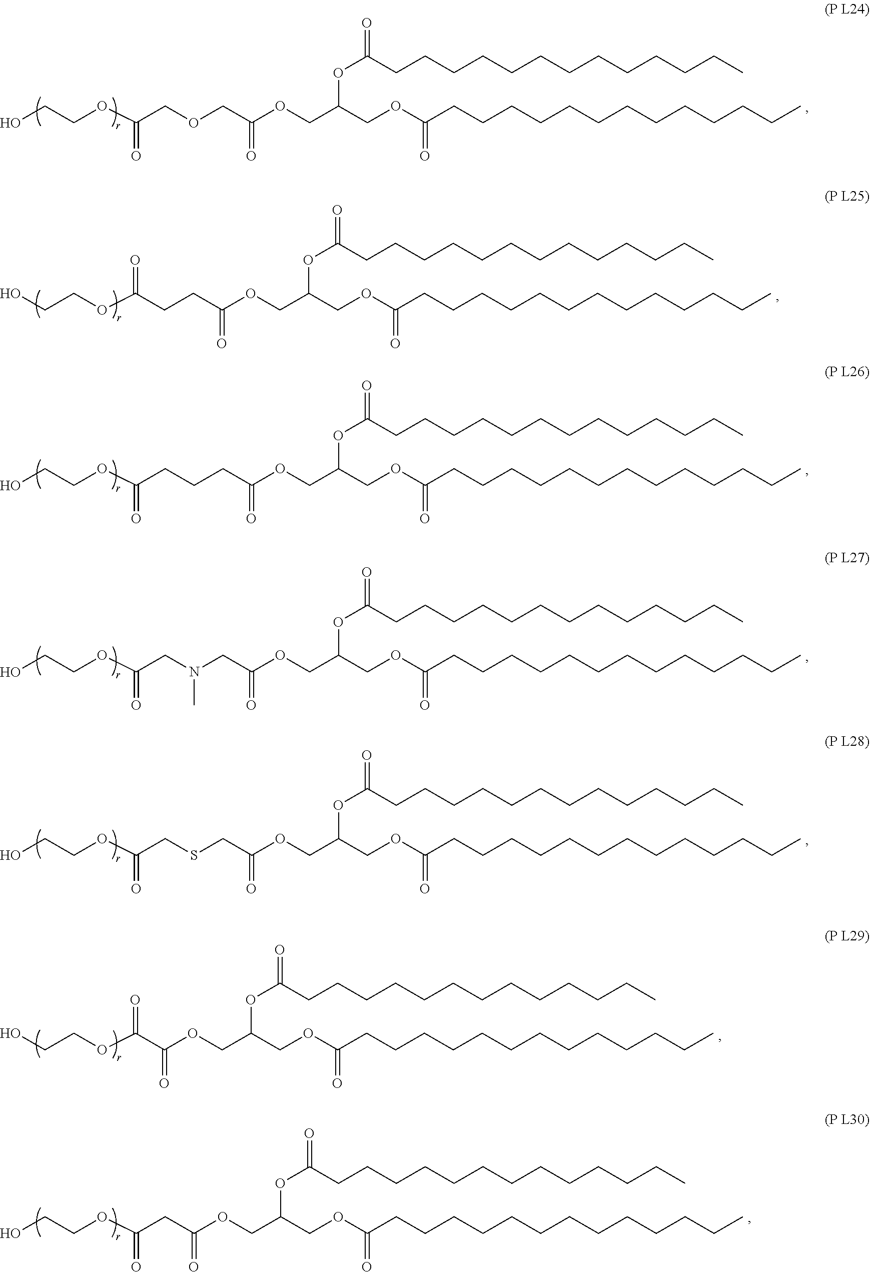

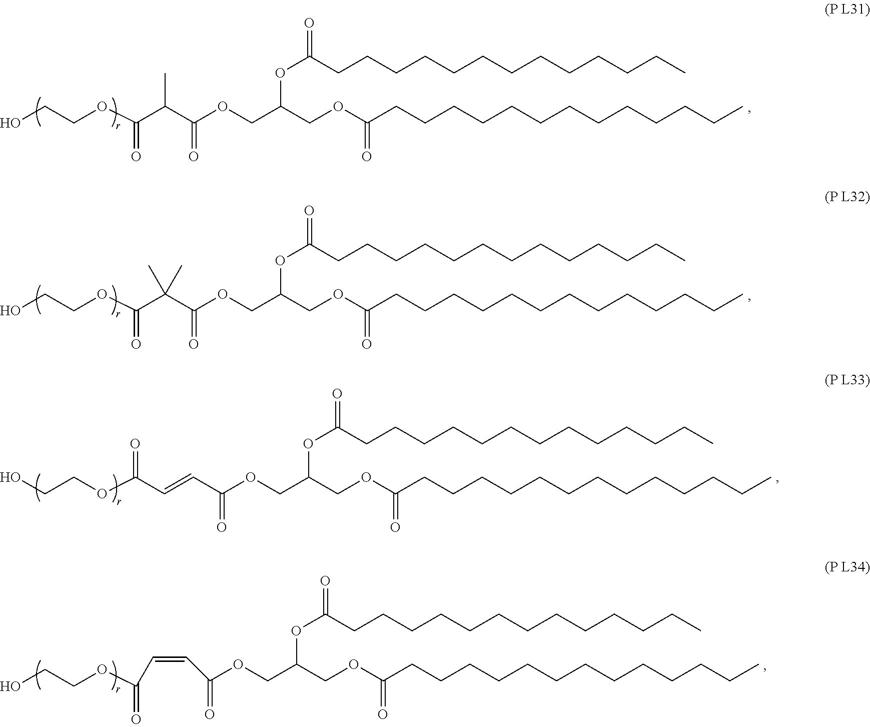

and pharmaceutically acceptable salts thereof.

In any of the foregoing or related aspects, a PEG lipid of the invention is featured wherein r is 40-50.

The LNPs provided herein, in certain embodiments, exhibit increased PEG shedding compared to existing LNP formulations comprising PEG lipids. “PEG shedding,” as used herein, refers to the cleavage of a PEG group from a PEG lipid. In many instances, cleavage of a PEG group from a PEG lipid occurs through serum-driven esterase-cleavage or hydrolysis. The PEG lipids provided herein, in certain embodiments, have been designed to control the rate of PEG shedding. In certain embodiments, an LNP provided herein exhibits greater than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits greater than 50% PEG shedding after about 6 hours in human serum. In certain embodiments, an LNP provided herein exhibits greater than 60% PEG shedding after about 6 hours in human serum. In certain embodiments, an LNP provided herein exhibits greater than 70% PEG shedding after about 6 hours in human serum. In certain embodiments, the LNP exhibits greater than 80% PEG shedding after about 6 hours in human serum. In certain embodiments, the LNP exhibits greater than 90% PEG shedding after about 6 hours in human serum. In certain embodiments, an LNP provided herein exhibits greater than 90% PEG shedding after about 6 hours in human serum.

In other embodiments, an LNP provided herein exhibits less than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits less than 60% PEG shedding after about 6 hours in human serum. In certain embodiments, an LNP provided herein exhibits less than 70% PEG shedding after about 6 hours in human serum. In certain embodiments, an LNP provided herein exhibits less than 80% PEG shedding after about 6 hours in human serum.

In addition to the PEG lipids provided herein, the LNP may comprise one or more additional lipid components. In certain embodiments, the PEG lipids are present in the LNP in a molar ratio of 0.15-15% with respect to other lipids. In certain embodiments, the PEG lipids are present in a molar ratio of 0.15-5% with respect to other lipids. In certain embodiments, the PEG lipids are present in a molar ratio of 1-5% with respect to other lipids. In certain embodiments, the PEG lipids are present in a molar ratio of 0.15-2% with respect to other lipids. In certain embodiments, the PEG lipids are present in a molar ratio of 1-2% with respect to other lipids. In certain embodiments, the PEG lipids are present in a molar ratio of approximately 1%, 1.1%, 1.2%, 1.3%, 1.4%, 1.5%, 1.6%, 1.7%, 1.8%, 1.9%, or 2% with respect to other lipids. In certain embodiments, the PEG lipids are present in a molar ratio of approximately 1.5% with respect to other lipids.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

Exemplary Synthesis:

Compound: HO-PEG₂₀₀₀-ester-C18

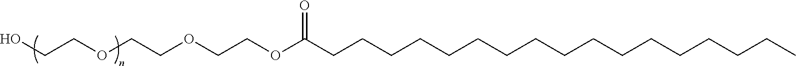

To a nitrogen filled flask containing palladium on carbon (10 wt. %, 74 mg, 0.070 mmol) was added Benzyl-PEG2000-ester-C18 (822 mg, 0.35 mmol) and MeOH (20 mL). The flask was evacuated nad backfilled with H₂ three times, and allowed to stir at RT and 1 atm H₂ for 12 hours. The mixture was filtered through celite, rinsing with DCM, and the filtrate was concentrated in vacuo to provide the desired product (692 mg, 88%). Using this methodology n=40-50. In one embodiment, n of the resulting polydispersed mixture is referred to by the average, 45.

For example, the value of r can be determined on the basis of a molecular weight of the PEG moiety within the PEG lipid. For example, a molecular weight of 2,000 (e.g., PEG2000) corresponds to a value of n of approximately 45. For a given composition, the value for n can connote a distribution of values within an art-accepted range, since polymers are often found as a distribution of different polymer chain lengths. For example, a skilled artisan understanding the polydispersity of such polymeric compositions would appreciate that an n value of 45 (e.g., in a structural formula) can represent a distribution of values between 40-50 in an actual PEG-containing composition, e.g., a DMG PEG2000 peg lipid composition.

In some aspects, an immune cell delivery lipid of the pharmaceutical compositions disclosed herein does not comprise a PEG-lipid.

In one embodiment, an immune cell delivery LNP of the disclosure comprises a PEG-lipid. In one embodiment, the PEG lipid is not PEG DMG. In some aspects, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some aspects, the PEG lipid is selected from the group consisting of PEG-c-DOMG, PEG-DMG, PEG-DLPE, PEG-DMPE, PEG-DPPC and PEG-DSPE lipid. In other aspects, the PEG-lipid is PEG-DMG.

In one embodiment, an immune cell delivery LNP of the disclosure comprises a PEG-lipid which has a chain length longer than about 14 or than about 10, if branched.

In one embodiment, the PEG lipid is a compound selected from the group consisting of any of Compound Nos. P415, P416, P417, P 419, P 420, P 423, P 424, P 428, P L1, P L2, P L16, P L17, P L18, P L19, P L22 and P L23. In one embodiment, the PEG lipid is a compound selected from the group consisting of any of Compound Nos. P415, P417, P 420, P 423, P 424, P 428, P L1, P L2, P L16, P L17, P L18, P L19, P L22 and P L23.

In one embodiment, a PEG lipid is selected from the group consisting of: Cmpd 428, PL16, PL17, PL 18, PL19, PL 1, and PL 2.

Immune Cell Delivery Potentiating Lipids

An effective amount of the immune cell delivery potentiating lipid in an LNP enhances delivery of the agent to an immune cell (e.g., a human or primate immune cell) relative to an LNP lacking the immune cell delivery potentiating lipid, thereby creating an immune cell delivery LNP. Immune cell delivery potentiating lipids can be characterized in that, when present in an LNP, they promote delivery of the agent present in the LNP to immune cells as compared to a control LNP lacking the immune cell delivery potentiating lipid.

In one embodiment, the presence of at least one immune cell delivery potentiating lipid in an LNP results in an increase in the percentage of LNPs associated with immune cells as compared to a control LNP lacking at least one immune cell delivery potentiating lipid. In another embodiment, the presence of at least one immune cell delivery potentiating lipid in an LNP results in an increase in the delivery of a nucleic acid molecule agent to immune cells as compared to a control LNP lacking the immune cell delivery potentiating lipid. In one embodiment, the presence of at least one immune cell delivery potentiating lipid in an LNP results in an increase in the delivery of a nucleic acid molecule agent to B cells as compared to a control LNP lacking the immune cell delivery potentiating lipid. In particular, in one embodiment, the presence of at least one immune cell delivery potentiating lipid in an LNP results in an increase in the delivery of a nucleic acid molecule agent to myeloid cells as compared to a control LNP lacking the immune cell delivery potentiating lipid. In one embodiment, the presence of at least one immune cell delivery potentiating lipid in an LNP results in an increase in the delivery of a nucleic acid molecule agent to T cells as compared to a control LNP lacking the immune cell delivery potentiating lipid.

In one embodiment, the presence of at least one immune cell delivery potentiating lipid in an LNP results in an increase in the percentage of LNPs binding to C1q as compared to a control LNP lacking at least one immune cell delivery potentiating lipid. In one embodiment, the presence of at least one immune cell delivery potentiating lipid in an LNP results in an increase in the percentage of C1q-bound LNPs taken up by immune cells (e.g., opsonized by immune cells) as compared to a control LNP lacking at least one immune cell delivery potentiating lipid.

In one embodiment, when the nucleic acid molecule is an mRNA, the presence of at least one immune cell delivery potentiating lipid results in at least about 2-fold greater expression of a protein molecule encoded by the mRNA in immune cells (e.g., a T cells, B cells, monocytes) as compared to a control LNP lacking the immune cell delivery potentiating lipid.

In one embodiment, an immune cell delivery potentiating lipid is an ionizable lipid. In any of the foregoing or related aspects, the ionizable lipid (denoted by I) of the LNP of the disclosure comprises a compound included in any e.g. a compound having any of Formula (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8) and/or any of Compounds X, Y, I 48, I 50, I 109, I 111, I 113, I 181, I 182, I 244, I 292, I 301, I 321, I 322, I 326, I 328, I 330, I 331, I 332 or I M.

In one embodiment, an immune cell delivery potentiating lipid is an ionizable lipid. In any of the foregoing or related aspects, the ionizable lipid of the LNP of the disclosure comprises a compound described herein as Compound X, Compound Y, Compound I-321, Compound I-292, Compound I-326, Compound I-182, Compound I-301, Compound I-48, Compound I-50, Compound I-328, Compound I-330, Compound I-109, Compound I-111 or Compound I-181.

In any of the foregoing or related aspects, the ionizable lipid of the LNP of the disclosure comprises at least one compound selected from the group consisting of: Compound Nos. I 18 (also referred to as Compound X), I 25 (also referred to as Compound Y), I 48, I 50, I 109, I 111, 1113, I 181, I 182, I 244, I 292, I 301, I 309, I 317, I 321, I 322, I 326, I 328, I 330, I 331, I 332, I 347, I 348, I 349, I 350, I 351 and I 352. In another embodiment, the ionizable lipid of the LNP of the disclosure comprises a compound selected from the group consisting of: Compound Nos. I 18 (also referred to as Compound X), I 25 (also referred to as Compound Y), I 48, I 50, I 109, I 111, I 181, I 182, I 292, I 301, I 321, I 326, I 328, and I 330. In another embodiment, the ionizable lipid of the LNP of the disclosure comprises a compound selected from the group consisting of: Compound Nos. I 182, I 301, I 321, and I 326.

It will be understood that in embodiments where the immune cell delivery potentiating lipid comprises an ionizable lipid, it may be the only ionizable lipid present in the LNP or it may be present as a blend with at least one additional ionizable lipid. That is to say that a blend of ionizable lipids (e.g., more than one that have immune cell delivery potentiating effects or one that has an immune cell delivery potentiating effect and at least one that does not) may be employed.

In one embodiment, an immune cell delivery potentiating lipid comprises a sterol. In another embodiment, an immune cell delivery potentiating lipid comprises a naturally occurring sterol. In another embodiment, an immune cell delivery potentiating lipid comprises a modified sterol. In one embodiment, an immune cell delivery potentiating lipid comprises one or more phytosterols. In one embodiment, the immune cell delivery potentiating lipid comprises a phytosterol/cholesterol blend.

In one embodiment, the immune cell delivery potentiating lipid comprises an effective amount of a phytosterol.

The term “phytosterol” refers to the group of plant based sterols and stanols that are phytosteroids including salts or esters thereof.

The term “sterol” refers to the subgroup of steroids also known as steroid alcohols. Sterols are usually divided into two classes: (1) plant sterols also known as “phytosterols”, and (2) animal sterols also known as “zoosterols” such as cholesterol. The term “stanol” refers to the class of saturated sterols, having no double bonds in the sterol ring structure.

The term “effective amount of phytosterol” is intended to mean an amount of one or more phytosterols in a lipid-based composition, including an LNP, that will elicit a desired activity (e.g., enhanced delivery, enhanced immune cell uptake, enhanced nucleic acid activity). In some embodiments, an effective amount of phytosterol is all or substantially all (i.e., about 99-100%) of the sterol in a lipid nanoparticle. In some embodiments, an effective amount of phytosterol is less than all or substantially all of the sterol in a lipid nanoparticle (less than about 99-100%), but greater than the amount of non-phytosterol sterol in the lipid nanoparticle. In some embodiments, an effective amount of phytosterol is greater than 50%, greater than 60%, greater than 70%, greater than 75%, greater than 80%, greater than 85%, greater than 90% or greater than 95% the total amount of sterol in a lipid nanoparticle. In some embodiments, an effective amount of phytosterol is 95-100%, 75-100%, or 50-100% of the total amount of sterol in a lipid nanoparticle.

In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenaserol, Δ7-avenaserol or a Δ7-stigmasterol, including analogs, salts or esters thereof, alone or in combination. In some embodiments, the phytosterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol.

In some embodiments, the sitosterol is a beta-sitosterol.

In some embodiments, the beta-sitosterol has the formula:

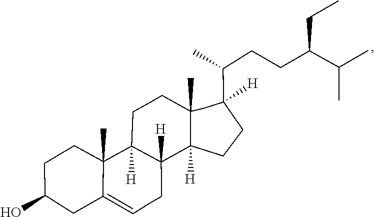

including analogs, salts or esters thereof.

In some embodiments, the sitosterol is a stigmasterol.

In some embodiments, the stigmasterol has the formula:

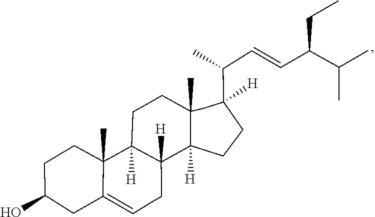

including analogs, salts or esters thereof.

In some embodiments, the sitosterol is a campesterol.

In some embodiments, the campesterol has the formula:

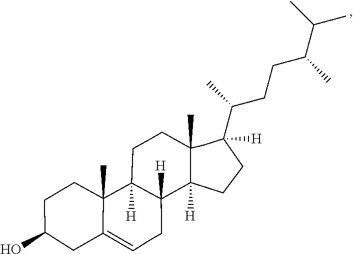

including analogs, salts or esters thereof.

In some embodiments, the sitosterol is a sitostanol.

In some embodiments, the sitostanol has the formula:

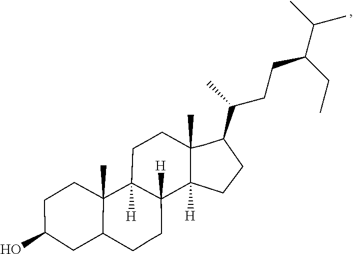

including analogs, salts or esters thereof.

In some embodiments, the sitosterol is a campestanol.

In some embodiments, the campestanol has the formula:

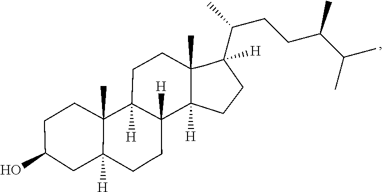

including analogs, salts or esters thereof.

In some embodiments, the sitosterol is a brassicasterol.

In some embodiments, the brassicasterol has the formula:

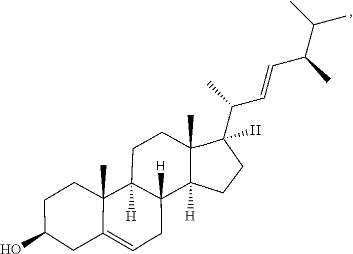

including analogs, salts or esters thereof.

In some embodiments, the sitosterol is a fucosterol.

In some embodiments, the fucosterol has the formula:

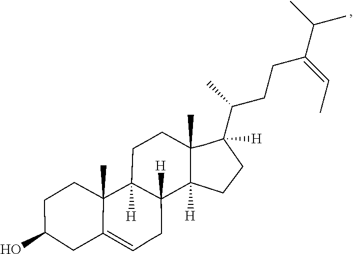

including analogs, salts or esters thereof.

In some embodiments, the phytosterol (e.g., beta-sitosterol) has a purity of greater than 70%. In some embodiments, the phytosterol (e.g., beta-sitosterol) has a purity of greater than 80%. In some embodiments, the phytosterol (e.g., beta-sitosterol) has a purity of greater than 90%. In some embodiments, the phytosterol (e.g., beta-sitosterol) has a purity of greater than 95%. In some embodiments, the phytosterol (e.g., beta-sitosterol) has a purity of greater than 97%, 98% or 99%.

In one embodiment, an immune cell delivery enhancing LNP comprises more than one type of structural lipid.

For example, in one embodiment, the immune cell delivery enhancing LNP comprises at least one immune cell delivery potentiating lipid which is a phytosterol. In one embodiment, the phytosterol is the only structural lipid present in the LNP. In another embodiment, the immune cell delivery LNP comprises a blend of structural lipids.

In one embodiment, the combined amount of the phytosterol and structural lipid (e.g., beta-sitosterol and cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one embodiment, the combined amount of the phytosterol and structural lipid (e.g., beta-sitosterol and cholesterol) in the lipid composition disclosed herein ranges from about 25 mol % to about 30 mol %, from about 30 mol % to about 35 mol %, or from about 35 mol % to about 40 mol %.

In one embodiment, the amount of the phytosterol and structural lipid (e.g., beta-sitosterol and cholesterol) in the lipid composition disclosed herein is about 24 mol %, about 29 mol %, about 34 mol %, or about 39 mol %.

In some embodiments, the combined amount of the phytosterol and structural lipid (e.g., beta-sitosterol and cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

In some embodiments, the lipid nanoparticle comprises one or more phytosterols (e.g., beta-sitosterol) and one or more structural lipids (e.g. cholesterol). In some embodiments, the mol % of the structural lipid is between about 1% and 50% of the mol % of phytosterol present in the lipid nanoparticle. In some embodiments, the mol % of the structural lipid is between about 10% and 40% of the mol % of phytosterol present in the lipid-based composition (e.g., LNP). In some embodiments, the mol % of the structural lipid is between about 20% and 30% of the mol % of phytosterol present in the lipid-based composition (e.g., LNP). In some embodiments, the mol % of the structural lipid is about 30% of the mol % of phytosterol present in the lipid-based composition (e.g., lipid nanoparticle).

In some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17, 18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

In certain embodiments, the LNP comprises 50% ionizable lipid, 10% helper lipid (e.g., phospholipid), 38.5% structural lipid, and 1.5% PEG lipid. In certain embodiments, the LNP comprises 50% ionizable lipid, 10% helper lipid (e.g., phospholipid), 38% structural lipid, and 2% PEG lipid. In certain embodiments, the LNP comprises 50% ionizable lipid, 20% helper lipid (e.g., phospholipid), 28.5% structural lipid, and 1.5% PEG lipid. In certain embodiments, the LNP comprises 50% ionizable lipid, 20% helper lipid (e.g., phospholipid), 28% structural lipid, and 2% PEG lipid. In certain embodiments, the LNP comprises 40% ionizable lipid, 30% helper lipid (e.g., phospholipid), 28.5% structural lipid, and 1.5% PEG lipid. In certain embodiments, the LNP comprises 40% ionizable lipid, 30% helper lipid (e.g., phospholipid), 28% structural lipid, and 2% PEG lipid. In certain embodiments, the LNP comprises 45% ionizable lipid, 20% helper lipid (e.g., phospholipid), 33.5% structural lipid, and 1.5% PEG lipid. In certain embodiments, the LNP comprises 45% ionizable lipid, 20% helper lipid (e.g., phospholipid), 33% structural lipid, and 2% PEG lipid.

In one aspect, the immune cell delivery enhancing LNP comprises phytosterol and the LNP does not comprise an additional structural lipid. Accordingly, the structural lipid (sterol) component of the LNP consists of phytosterol. In another aspect, the immune cell delivery enhancing LNP comprises phytosterol and an additional structural lipid. Accordingly, the sterol component of the LNP comprise phytosterol and one or more additional sterols or structural lipids.

In any of the foregoing or related aspects, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), brassicasterol or stigmasterol, or combinations or blends thereof. In another embodiment, the structural lipid (e.g., sterol, such as a phytosterol or phytosterol/cholesterol blend) of the LNP of the disclosure comprises a compound selected from cholesterol, β-sitosterol, campesterol, β-sitostanol, brassicasterol, stigmasterol, β-sitosterol-d7, Compound S-30, Compound S-31, Compound S-32, or combinations or blends thereof. In another embodiment, the structural lipid (e.g., sterol, such as a phytosterol or phytosterol/cholesterol blend) of the LNP of the disclosure comprises a compound described herein as cholesterol, β-sitosterol (also referred to herein as Cmpd S 141), campesterol (also referred to herein as Cmpd S 143), β-sitostanol (also referred to herein as Cmpd S 144), Compound S-140, Compound S-144, brassicasterol (also referred to herein as Cmpd S 148) or Composition S-183 (˜40% Compound S-141, ˜25% Compound S-140, ˜25% Compound S-143 and ˜10% brassicasterol). In some embodiments, the structural lipid of the LNP of the disclosure comprises a compound described herein as Compound S-159, Compound S-160, Compound S-164, Compound S-165, Compound S-167, Compound S-170, Compound S-173 or Compound S-175.

In one embodiment, an immune cell delivery enhancing LNP comprises a non-cationic helper lipid, e.g., phospholipid. In any of the foregoing or related aspects, the non-cationic helper lipid (e.g., phospholipid) of the LNP of the disclosure comprises a compound described herein as DSPC, DMPE, DOPC or H-409. In one embodiment, the non-cationic helper lipid, e.g., phospholipid is DSPC. In other embodiments, the non-cationic helper lipid (e.g., phospholipid) of the LNP of the disclosure comprises a compound described herein as DSPC, DMPE, DOPC, DPPC, PMPC, H-409, H-418, H-420, H-421 or H-422.

In any of the foregoing or related aspects, the PEG lipid of the LNP of the disclosure comprises a compound described herein can be selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In another embodiment, the PEG lipid is selected from the group consisting of Compound Nos. P415, P416, P417, P 419, P 420, P 423, P 424, P 428, P L5, P L1, P L2, P L16, P L17, P L18, P L19, P L22, P L23, DMG, DPG and DSG. In another embodiment, the PEG lipid is selected from the group consisting of Cmpd 428, PL16, PL17, PL 18, PL19, P L5, PL 1, and PL 2.

In one embodiment, an immune cell delivery potentiating lipid comprises an effective amount of a combination of an ionizable lipid and a phytosterol.

In other embodiments, the disclosure provides lipid nanoparticles comprising one or more immune cell delivery potentiating lipids, wherein the LNP comprises Compound X as the ionizable lipid, DSPC as the phospholipid, cholesterol or a cholesterol/β-sitosterol blend as the structural lipid and Compound 428 as the PEG lipid. In various embodiments of these Compound X-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol. In another embodiment, the structural lipid is cholesterol/β-sitosterol at a total percentage of 38.5%, wherein the blend can comprise, for example: (i) 20% cholesterol and 18.5% β-sitosterol; or (ii) 10% cholesterol and 28.5% β-sitosterol.

In other embodiments, the disclosure provides lipid nanoparticles comprising one or more immune cell delivery potentiating lipids, wherein the LNP comprises Compound Y as the ionizable lipid, DSPC as the phospholipid, cholesterol or a cholesterol/β-sitosterol blend as the structural lipid and Compound 428 as the PEG lipid. In various embodiments of these Compound Y-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-182 as the ionizable lipid, DSPC as the phospholipid, cholesterol or a cholesterol/β-sitosterol blend as the structural lipid and Compound 428 as the PEG lipid. In various embodiments of these Compound I-182-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-321 as the ionizable lipid, DSPC as the phospholipid, cholesterol or a cholesterol/β-sitosterol blend as the structural lipid and Compound 428 as the PEG lipid. In various embodiments of these Compound I-321-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-292 as the ionizable lipid, DSPC as the phospholipid, cholesterol or a cholesterol/β-sitosterol blend as the structural lipid and Compound 428 as the PEG lipid. In various embodiments of these Compound I-292-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-326 as the ionizable lipid, DSPC as the phospholipid, cholesterol or a cholesterol/β-sitosterol blend as the structural lipid and Compound 428 as the PEG lipid. In various embodiments of these Compound I-326-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-301 as the ionizable lipid, DSPC as the phospholipid, cholesterol or a cholesterol/β-sitosterol blend as the structural lipid and Compound 428 as the PEG lipid. In various embodiments of these Compound I-301-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-48 as the ionizable lipid, DSPC as the phospholipid, cholesterol or a cholesterol/β-sitosterol blend as the structural lipid and Compound 428 as the PEG lipid. In various embodiments of these Compound I-48-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-50 as the ionizable lipid, DSPC as the phospholipid, cholesterol or a cholesterol/β-sitosterol blend as the structural lipid and Compound 428 as the PEG lipid. In various embodiments of these Compound I-50-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-328 as the ionizable lipid, DSPC as the phospholipid, cholesterol or a cholesterol/β-sitosterol blend as the structural lipid and Compound 428 as the PEG lipid. In various embodiments of these Compound I-328-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-330 as the ionizable lipid, DSPC as the phospholipid, cholesterol or a cholesterol/β-sitosterol blend as the structural lipid and Compound 428 as the PEG lipid. In various embodiments of these Compound I-330-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-109 as the ionizable lipid, DSPC as the phospholipid, cholesterol or a cholesterol/β-sitosterol blend as the structural lipid and Compound 428 as the PEG lipid. In various embodiments of these Compound I-109-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-111 as the ionizable lipid, DSPC as the phospholipid, cholesterol or a cholesterol/β-sitosterol blend as the structural lipid and Compound 428 as the PEG lipid. In various embodiments of these Compound I-111-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises Compound I-181 as the ionizable lipid, DSPC as the phospholipid, cholesterol or a cholesterol/β-sitosterol blend as the structural lipid and Compound 428 as the PEG lipid. In various embodiments of these Compound I-181-containing compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2. For the structural lipid component, in one embodiment the structural lipid is entirely cholesterol at 38% or 28%. In another embodiment, the structural lipid is cholesterol/β-sitosterol at a total percentage of 38% or 28%, wherein the blend can comprise, for example: (i) 20% cholesterol and 18% β-sitosterol; (ii) 10% cholesterol and 18% β-sitosterol or (iii) 10% cholesterol and 28% β-sitosterol.

In other embodiments, the disclosure provides lipid nanoparticles comprising one or more immune cell delivery potentiating lipids, wherein the LNP comprises any of Compounds X, Y, I-321, I-292, I-326, I-182, I-301, I-48, I-50, I-328, I-330, I-109, I-111 or I-181 as the ionizable lipid; DSPC as the phospholipid; cholesterol, a cholesterol/β-sitosterol blend, a β-sitosterol/β-sitostanol blend, a β-sitosterol/camposterol blend, a β-sitosterol/β-sitostanol/camposterol blend, a cholesterol/camposterol blend, a cholesterol/β-sitostanol blend, a cholesterol/β-sitostanol/camposterol blend or a cholesterol/β-sitosterol/β-sitostanol/camposterol blend as the structural lipid; and Compound 428 as the PEG lipid. In various embodiments of these compositions, the ratios of the ionizable lipid:phospholipid:structural lipid:PEG lipid can be, for example, as follows: (i) 50:10:38:2; (ii) 50:20:28:2; (iii) 40:20:38:2; (iv) 40:30:28:2; (v) 40:18.5:40:1.5; or (vi) 45:20:33.5:1.5. In one embodiment, for the structural lipid component, the LNP can comprise, for example, 40% structural lipid composed of (i) 10% cholesterol and 30% β-sitosterol; (ii) 10% cholesterol and 30% campesterol; (iii) 10% cholesterol and 30% β-sitostanol; (iv) 10% cholesterol, 20% β-sitosterol and 10% campesterol; (v) 10% cholesterol, 20% 3-sitosterol and 10% β-sitostanol; (vi) 10% cholesterol, 10% β-sitosterol and 20% campesterol; (vii) 10% cholesterol, 10% β-sitosterol and 20% campesterol; (viii) 10% cholesterol, 20% campesterol and 10% β-sitostanol; (ix) 10% cholesterol, 10% campesterol and 20% β-sitostanol; or (x) 10% cholesterol, 10% β-sitosterol, 10% campesterol and 10% β-sitostanol. In another embodiment, for the structural lipid component, the LNP can comprise, for example, 33.5% structural lipid composed of (i) 33.5% cholesterol; (ii) 18.5% cholesterol, 15% β-sitosterol; (iii) 18.5% cholesterol, 15% campesterol; or (iv) 18.5% cholesterol, 15% campesterol.

In other embodiments, the disclosure provides lipid nanoparticles comprising one or more immune cell delivery potentiating lipids, wherein the LNP comprises Compound I-301, Compound I-321 or Compound I-326 as the ionizable lipid; DSPC as the phospholipid; cholesterol or a cholesterol/β-sitosterol blend as the structural lipid; and Compound 428 as the PEG lipid. In one embodiment, the LNP enhances delivery to T cells (e.g., CD3+ T cells).

In other embodiment, the disclosure provides lipid nanoparticles comprising one or more immune cell delivery potentiating lipids, wherein the LNP comprises Compound X, Compound I-109, Compound I-111, Compound I-181, Compound I-182 or Compound I-244, wherein the LNP enhances delivery to monocytes. The other components of the LNP can be selected from those disclosed herein, for example DSPC as the phospholipid; cholesterol or a cholesterol/β-sitosterol blend as the structural lipid; and Compound 428 as the PEG lipid.

In other embodiment, the disclosure provides lipid nanoparticles comprising one or more immune cell delivery potentiating lipids, wherein the LNP comprises camposterol, β-sitostanol or stigmasterol as the structural lipid, wherein the LNP enhances delivery to monocytes. The other components of the LNP can be selected from those disclosed herein, for example Compound X, Compound I-109, Compound I-111, Compound I-181, Compound I-182 or Compound I-244 as the ionizable lipid; DSPC as the phospholipid; and Compound 428 as the PEG lipid.

In other embodiment, the disclosure provides lipid nanoparticles comprising one or more immune cell delivery potentiating lipids, wherein the LNP comprises DOPC, DMPE or H-409 as the helper lipid (e.g., phospholipid), wherein the LNP enhances delivery to monocytes. The other components of the LNP can be selected from those disclosed herein, for example Compound X, Compound I-109, Compound I-111, Compound I-181, Compound I-182 or Compound I-244 as the ionizable lipid; cholesterol, a cholesterol/β-sitosterol blend, camposterol, β-sitostanol or stigmasterol as the structural lipid; and Compound 428 as the PEG lipid.

### Exemplary Additional LNP Components

Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer. For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10⁻⁴ M in water at about 30° C. and atmospheric pressure.

For example, amphiphilic polymer has a critical micelle concentration (CMC) ranging between about 0.1×10⁻⁴ M and about 1.3×10⁻⁴ M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

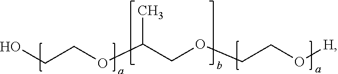

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof.

For example, the polyethylene glycol ether is a compound of Formula (VIII):

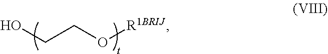

or a salt or isomer thereof, wherein:

t is an integer between 1 and 100;

R^{1BRIJ} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and

each instance of R^{N} is independently hydrogen, C₁₋₆ alkyl, or a nitrogen protecting group

In some embodiment, R^{1BRIJ} is C₁₈ alkyl. For example, the polyethylene glycol ether is a compound of Formula (VIII-a):

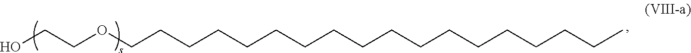

or a salt or isomer thereof.

In some embodiments, R^{1BRIJ} is C₁₈ alkenyl. For example, the polyethylene glycol ether is a compound of Formula (VIII-b):

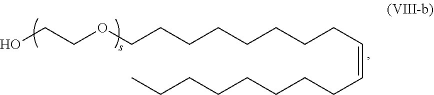

or a salt or isomer thereof

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween® 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

Adjuvants

In some embodiments, an LNP of the invention optionally includes one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

Other Components

An LNP of the invention may optionally include one or more components in addition to those described in the preceding sections. For example, a lipid nanoparticle may include one or more small hydrophobic molecules such as a vitamin (e.g., vitamin A or vitamin E) or a sterol.

Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### LNP Compositions

A lipid nanoparticle described herein may be designed for one or more specific applications or targets. The elements of a lipid nanoparticle and their relative amounts may be selected based on a particular application or target, and/or based on the efficacy, toxicity, expense, ease of use, availability, or other feature of one or more elements. Similarly, the particular formulation of a lipid nanoparticle may be selected for a particular application or target according to, for example, the efficacy and toxicity of particular combinations of elements. The efficacy and tolerability of a lipid nanoparticle formulation may be affected by the stability of the formulation.

The LNPs of the invention comprise at least one immune cell delivery potentiating lipid. The subject LNPs comprise: an effective amount of an immune cell delivery potentiating lipid as a component of an LNP, wherein the LNP comprises an (i) ionizable lipid; (ii) cholesterol or other structural lipid; (iii) a non-cationic helper lipid or phospholipid; a (iv) PEG lipid and (v) an agent (e.g., an nucleic acid molecule) encapsulated in and/or associated with the LNP, wherein the effective amount of the immune cell delivery potentiating lipid enhances delivery of the agent to an immune cell (e.g., a human or primate immune cell) relative to an LNP lacking the immune cell delivery potentiating lipid.

The elements of the various components may be provided in specific fractions, e.g., mole percent fractions.

For example, in any of the foregoing or related aspects, the LNP of the disclosure comprises a structural lipid or a salt thereof. In some aspects, the structural lipid is cholesterol or a salt thereof. In further aspects, the mol % cholesterol is between about 1% and 50% of the mol % of phytosterol present in the LNP. In other aspects, the mol % cholesterol is between about 10% and 40% of the mol % of phytosterol present in the LNP. In some aspects, the mol % cholesterol is between about 20% and 30% of the mol % of phytosterol present in the LNP. In further aspects, the mol % cholesterol is about 30% of the mol % of phytosterol present in the LNP.

In any of the foregoing or related aspects, the LNP of the disclosure comprises about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.

In any of the foregoing or related aspects, the LNP of the disclosure comprises about 35 mol % to about 55 mol % ionizable lipid, about 5 mol % to about 25 mol % phospholipid, about 30 mol % to about 40 mol % sterol, and about 0 mol % to about 10 mol % PEG lipid.

In any of the foregoing or related aspects, the LNP of the disclosure comprises about 50 mol % ionizable lipid, about 10 mol % phospholipid, about 38.5 mol % sterol, and about 1.5 mol % PEG lipid.

In certain embodiments, the ionizable lipid component of the lipid nanoparticle includes about 30 mol % to about 60 mol % ionizable lipid, about 0 mol % to about 30 mol % non-cationic helper lipid, about 18.5 mol % to about 48.5 mol % phytosterol optionally including one or more structural lipids, and about 0 mol % to about 10 mol % of PEG lipid, provided that the total mol % does not exceed 100%. In some embodiments, the ionizable lipid component of the lipid nanoparticle includes about 35 mol % to about 55 mol % ionizable lipid, about 5 mol % to about 25 mol % non-cationic helper lipid, about 30 mol % to about 40 mol % phytosterol optionally including one or more structural lipids, and about 0 mol % to about 10 mol % of PEG lipid. In a particular embodiment, the lipid component includes about 50 mol % ionizable lipid, about 10 mol % non-cationic helper lipid, about 38.5 mol % phytosterol optionally including one or more structural lipids, and about 1.5 mol % of PEG lipid. In another particular embodiment, the lipid component includes about 40 mol % ionizable lipid, about 20 mol % non-cationic helper lipid, about 38.5 mol % phytosterol optionally including one or more structural lipids, and about 1.5 mol % of PEG lipid. In some embodiments, the phytosterol may be beta-sitosterol, the non-cationic helper lipid may be a phospholipid such as DOPE, DSPC or a phospholipid substitute such as oleic acid. In other embodiments, the PEG lipid may be PEG-DMG and/or the structural lipid may be cholesterol.

In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and a structural lipid, and about 0 mol % to about 10 mol % PEG lipid. In some aspects, the LNP of the disclosure comprises about 30 mol % to about 60 mol % ionizable lipid, about 0 mol % to about 30 mol % non-cationic helper lipid, about 18.5 mol % to about 48.5 mol % phytosterol and cholesterol, and about 0 mol % to about 10 mol % PEG lipid.

In some aspects, the LNP of the disclosure comprises about 35 mol % to about 55 mol % ionizable lipid, about 5 mol % to about 25 mol % non-cationic helper lipid, about 30 mol % to about 40 mol % phytosterol, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and a structural lipid, and about 0 mol % to about 10 mol % PEG lipid. In some aspects, the LNP of the disclosure comprises about 35 mol % to about 55 mol % ionizable lipid, about 5 mol % to about 25 mol % non-cationic helper lipid, about 30 mol % to about 40 mol % phytosterol and cholesterol, and about 0 mol % to about 10 mol % PEG lipid.

In some aspects, the LNP of the disclosure comprises about 50 mol % ionizable lipid, about 10 mol % non-cationic helper lipid, about 38.5 mol % phytosterol, and about 1.5 mol % PEG lipid. In some aspects, the LNP of the disclosure comprises about 50 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 50 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 38.5 mol % phytosterol, and about 1.5 mol % PEG lipid. In some aspects, the LNP of the disclosure comprises about 40 mol % ionizable lipid, about 20 mol % non-cationic helper lipid, about 38.5 mol % phytosterol and a structural lipid, and about 1.5 mol % PEG lipid. In some aspects, the LNP of the disclosure comprises about 40 mol % ionizable lipid, about 20 mol % non-cationic helper lipid, about 38.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 38.5 mol % phytosterol, and about 1.5 mol % PEG lipid. In some aspects, the LNP of the disclosure comprises about 45 mol % ionizable lipid, about 10 mol % non-cationic helper lipid, about 38.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 38.5 mol % phytosterol and cholesterol, and about 1.5 mol % PEG lipid.

In some aspects, the LNP of the disclosure comprises about 55 mol % ionizable lipid, about 5 mol % non-cationic helper lipid, about 38.5 mol % phytosterol, and about 1.5 mol % PEG lipid. In some aspects, the LNP of the disclosure comprises about 55 mol % ionizable lipid, about 5 mol % non-cationic helper lipid, about 38.5 mol % phytosterol and a structural lipid, and about 1.5 mol % PEG lipid. In some aspects, the LNP of the disclosure comprises about 55 mol % ionizable lipid, about 5 mol % non-cationic helper lipid, about 38.5 mol % phytosterol and cholesterol, and about 1.5 mol % PEG lipid.

In some aspects, the LNP of the disclosure comprises about 60 mol % ionizable lipid, about 5 mol % non-cationic helper lipid, about 33.5 mol % phytosterol, and about 1.5 mol % PEG lipid. In some aspects, the LNP of the disclosure comprises about 60 mol % ionizable lipid, about 5 mol % non-cationic helper lipid, about 33.5 mol % phytosterol and a structural lipid, and about 1.5 mol % PEG lipid. In some aspects, the LNP of the disclosure comprises about 60 mol % ionizable lipid, about 5 mol % non-cationic helper lipid, about 33.5 mol % phytosterol and cholesterol, and about 1.5 mol % PEG lipid.

In some aspects, the LNP of the disclosure comprises about 45 mol % ionizable lipid, about 20 mol % non-cationic helper lipid, about 33.5 mol % phytosterol, and about 1.5 mol % PEG lipid. In some aspects, the LNP of the disclosure comprises about 45 mol % ionizable lipid, about 20 mol % non-cationic helper lipid, about 33.5 mol % phytosterol and a structural lipid, and about 1.5 mol % PEG lipid. In some aspects, the LNP of the disclosure comprises about 45 mol % ionizable lipid, about 20 mol % non-cationic helper lipid, about 33.5 mol % phytosterol and cholesterol, and about 1.5 mol % PEG lipid.

In some aspects, the LNP of the disclosure comprises about 50 mol % ionizable lipid, about 20 mol % non-cationic helper lipid, about 28.5 mol % phytosterol, and about 1.5 mol % PEG lipid. In some aspects, the LNP of the disclosure comprises about 50 mol % ionizable lipid, about 20 mol % non-cationic helper lipid, about 28.5 mol % phytosterol and a structural lipid, and about 1.5 mol % PEG lipid. In some aspects, the LNP of the disclosure comprises about 50 mol % ionizable lipid, about 20 mol % non-cationic helper lipid, about 28.5 mol % phytosterol and cholesterol, and about 1.5 mol % PEG lipid.

In some aspects, the LNP of the disclosure comprises about 55 mol % ionizable lipid, about 20 mol % non-cationic helper lipid, about 23.5 mol % phytosterol, and about 1.5 mol % PEG lipid. In some aspects, the LNP of the disclosure comprises about 55 mol % ionizable lipid, about 20 mol % non-cationic helper lipid, about 23.5 mol % phytosterol and a structural lipid, and about 1.5 mol % PEG lipid. In some aspects, the LNP of the disclosure comprises about 55 mol % ionizable lipid, about 20 mol % non-cationic helper lipid, about 23.5 mol % phytosterol and cholesterol, and about 1.5 mol % PEG lipid.

In some aspects, the LNP of the disclosure comprises about 60 mol % ionizable lipid, about 20 mol % non-cationic helper lipid, about 18.5 mol % phytosterol, and about 1.5 mol % PEG lipid. In some aspects, the LNP of the disclosure comprises about 60 mol % ionizable lipid, about 20 mol % non-cationic helper lipid, about 18.5 mol % phytosterol and a structural lipid, and about 1.5 mol % PEG lipid. In some aspects, the LNP of the disclosure comprises about 60 mol % ionizable lipid, about 20 mol % non-cationic helper lipid, about 18.5 mol % phytosterol and cholesterol, and about 1.5 mol % PEG lipid.

In some aspects, the LNP of the disclosure comprises about 40 mol % ionizable lipid, about 15 mol % non-cationic helper lipid, about 43.5 mol % phytosterol, and about 1.5 mol % PEG lipid. In some aspects, the LNP of the disclosure comprises about 40 mol % ionizable lipid, about 15 mol % non-cationic helper lipid, about 43.5 mol % phytosterol and a structural lipid, and about 1.5 mol % PEG lipid. In some aspects, the LNP of the disclosure comprises about 40 mol % ionizable lipid, about 15 mol % non-cationic helper lipid, about 43.5 mol % phytosterol and cholesterol, and about 1.5 mol % PEG lipid.

In some aspects, the LNP of the disclosure comprises about 50 mol % ionizable lipid, about 15 mol % non-cationic helper lipid, about 33.5 mol % phytosterol, and about 1.5 mol % PEG lipid. In some aspects, the LNP of the disclosure comprises about 50 mol % ionizable lipid, about 15 mol % non-cationic helper lipid, about 33.5 mol % phytosterol and a structural lipid, and about 1.5 mol % PEG lipid. In some aspects, the LNP of the disclosure comprises about 50 mol % ionizable lipid, about 15 mol % non-cationic helper lipid, about 33.5 mol % phytosterol and cholesterol, and about 1.5 mol % PEG lipid.

In some aspects, the LNP of the disclosure comprises about 55 mol % ionizable lipid, about 15 mol % non-cationic helper lipid, about 28.5 mol % phytosterol, and about 1.5 mol % PEG lipid. In some aspects, the LNP of the disclosure comprises about 55 mol % ionizable lipid, about 15 mol % non-cationic helper lipid, about 28.5 mol % phytosterol and a structural lipid, and about 1.5 mol % PEG lipid. In some aspects, the LNP of the disclosure comprises about 55 mol % ionizable lipid, about 15 mol % non-cationic helper lipid, about 28.5 mol % phytosterol and cholesterol, and about 1.5 mol % PEG lipid.

In some aspects, the LNP of the disclosure comprises about 60 mol % ionizable lipid, about 15 mol % non-cationic helper lipid, about 23.5 mol % phytosterol, and about 1.5 mol % PEG lipid. In some aspects, the LNP of the disclosure comprises about 60 mol % ionizable lipid, about 15 mol % non-cationic helper lipid, about 23.5 mol % phytosterol and a structural lipid, and about 1.5 mol % PEG lipid. In some aspects, the LNP of the disclosure comprises about 60 mol % ionizable lipid, about 15 mol % non-cationic helper lipid, about 23.5 mol % phytosterol and cholesterol, and about 1.5 mol % PEG lipid.

In some aspects, the LNP of the disclosure comprises about 40 mol % ionizable lipid, about 10 mol % non-cationic helper lipid, about 48.5 mol % phytosterol, and about 1.5 mol % PEG lipid. In some aspects, the LNP of the disclosure comprises about 40 mol % ionizable lipid, about 10 mol % non-cationic helper lipid, about 48.5 mol % phytosterol and a structural lipid, and about 1.5 mol % PEG lipid. In some aspects, the LNP of the disclosure comprises about 40 mol % ionizable lipid, about 10 mol % non-cationic helper lipid, about 48.5 mol % phytosterol and cholesterol, and about 1.5 mol % PEG lipid.

In some aspects, the LNP of the disclosure comprises about 45 mol % ionizable lipid, about 10 mol % non-cationic helper lipid, about 43.5 mol % phytosterol, and about 1.5 mol % PEG lipid. In some aspects, the LNP of the disclosure comprises about 45 mol % ionizable lipid, about 10 mol % non-cationic helper lipid, about 43.5 mol % phytosterol and a structural lipid, and about 1.5 mol % PEG lipid. In some aspects, the LNP of the disclosure comprises about 45 mol % ionizable lipid, about 10 mol % non-cationic helper lipid, about 43.5 mol % phytosterol and cholesterol, and about 1.5 mol % PEG lipid.

In some aspects, the LNP of the disclosure comprises about 55 mol % ionizable lipid, about 10 mol % non-cationic helper lipid, about 33.5 mol % phytosterol, and about 1.5 mol % PEG lipid. In some aspects, the LNP of the disclosure comprises about 55 mol % ionizable lipid, about 10 mol % non-cationic helper lipid, about 33.5 mol % phytosterol and a structural lipid, and about 1.5 mol % PEG lipid. In some aspects, the LNP of the disclosure comprises about 55 mol % ionizable lipid, about 10 mol % non-cationic helper lipid, about 33.5 mol % phytosterol and cholesterol, and about 1.5 mol % PEG lipid.

In some aspects, the LNP of the disclosure comprises about 60 mol % ionizable lipid, about 10 mol % non-cationic helper lipid, about 28.5 mol % phytosterol, and about 1.5 mol % PEG lipid. In some aspects, the LNP of the disclosure comprises about 60 mol % ionizable lipid, about 10 mol % non-cationic helper lipid, about 28.5 mol % phytosterol and a structural lipid, and about 1.5 mol % PEG lipid. In some aspects, the LNP of the disclosure comprises about 60 mol % ionizable lipid, about 10 mol % non-cationic helper lipid, about 28.5 mol % phytosterol and cholesterol, and about 1.5 mol % PEG lipid.

In some aspects, the LNP of the disclosure comprises about 40 mol % ionizable lipid, about 5 mol % non-cationic helper lipid, about 53.5 mol % phytosterol, and about 1.5 mol % PEG lipid. In some aspects, the LNP of the disclosure comprises about 40 mol % ionizable lipid, about 5 mol % non-cationic helper lipid, about 53.5 mol % phytosterol and a structural lipid, and about 1.5 mol % PEG lipid. In some aspects, the LNP of the disclosure comprises about 40 mol % ionizable lipid, about 5 mol % non-cationic helper lipid, about 53.5 mol % phytosterol and cholesterol, and about 1.5 mol % PEG lipid.

In some aspects, the LNP of the disclosure comprises about 45 mol % ionizable lipid, about 5 mol % non-cationic helper lipid, about 48.5 mol % phytosterol, and about 1.5 mol % PEG lipid. In some aspects, the LNP of the disclosure comprises about 45 mol % ionizable lipid, about 5 mol % non-cationic helper lipid, about 48.5 mol % phytosterol and a structural lipid, and about 1.5 mol % PEG lipid. In some aspects, the LNP of the disclosure comprises about 45 mol % ionizable lipid, about 5 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 5 mol % non-cationic helper lipid, about 43.5 mol % phytosterol, and about 1.5 mol % PEG lipid. In some aspects, the LNP of the disclosure comprises about 50 mol % ionizable lipid, about 5 mol % non-cationic helper lipid, about 43.5 mol % phytosterol and a structural lipid, and about 1.5 mol % PEG lipid. In some aspects, the LNP of the disclosure comprises about 50 mol % ionizable lipid, about 5 mol % non-cationic helper lipid, about 43.5 mol % phytosterol and cholesterol, and about 1.5 mol % PEG lipid.

In some aspects, the LNP of the disclosure comprises about 40 mol % ionizable lipid, about 20 mol % non-cationic helper lipid, about 40 mol % phytosterol, and about 0 mol % PEG lipid. In some aspects, the LNP of the disclosure comprises about 40 mol % ionizable lipid, about 20 mol % non-cationic helper lipid, about 40 mol % phytosterol and a structural lipid, and about 0 mol % PEG lipid. In some aspects, the LNP of the disclosure comprises about 40 mol % ionizable lipid, about 20 mol % non-cationic helper lipid, about 40 mol % phytosterol and cholesterol, and about 0 mol % PEG lipid.

In some aspects, the LNP of the disclosure comprises about 45 mol % ionizable lipid, about 20 mol % non-cationic helper lipid, about 35 mol % phytosterol, and about 0 mol % PEG lipid. In some aspects, the LNP of the disclosure comprises about 45 mol % ionizable lipid, about 20 mol % non-cationic helper lipid, about 35 mol % phytosterol and a structural lipid, and about 0 mol % PEG lipid. In some aspects, the LNP of the disclosure comprises about 45 mol % ionizable lipid, about 20 mol % non-cationic helper lipid, about 35 mol % phytosterol and cholesterol, and about 0 mol % PEG lipid.

In some aspects, the LNP of the disclosure comprises about 50 mol % ionizable lipid, about 20 mol % non-cationic helper lipid, about 30 mol % phytosterol, and about 0 mol % PEG lipid. In some aspects, the LNP of the disclosure comprises about 50 mol % ionizable lipid, about 20 mol % non-cationic helper lipid, about 30 mol % phytosterol and a structural lipid, and about 0 mol % PEG lipid. In some aspects, the LNP of the disclosure comprises about 50 mol % ionizable lipid, about 20 mol % non-cationic helper lipid, about 30 mol % phytosterol and cholesterol, and about 0 mol % PEG lipid.

In some aspects, the LNP of the disclosure comprises about 55 mol % ionizable lipid, about 20 mol % non-cationic helper lipid, about 25 mol % phytosterol, and about 0 mol % PEG lipid. In some aspects, the LNP of the disclosure comprises about 55 mol % ionizable lipid, about 20 mol % non-cationic helper lipid, about 25 mol % phytosterol and a structural lipid, and about 0 mol % PEG lipid. In some aspects, the LNP of the disclosure comprises about 55 mol % ionizable lipid, about 20 mol % non-cationic helper lipid, about 25 mol % phytosterol and cholesterol, and about 0 mol % PEG lipid.

In some aspects, the LNP of the disclosure comprises about 60 mol % ionizable lipid, about 20 mol % non-cationic helper lipid, about 20 mol % phytosterol, and about 0 mol % PEG lipid. In some aspects, the LNP of the disclosure comprises about 60 mol % ionizable lipid, about 20 mol % non-cationic helper lipid, about 20 mol % phytosterol and a structural lipid, and about 0 mol % PEG lipid. In some aspects, the LNP of the disclosure comprises about 60 mol % ionizable lipid, about 20 mol % non-cationic helper lipid, about 20 mol % phytosterol and cholesterol, and about 0 mol % PEG lipid.

In some aspects, the LNP of the disclosure comprises about 40 mol % ionizable lipid, about 15 mol % non-cationic helper lipid, about 45 mol % phytosterol, and about 0 mol % PEG lipid. In some aspects, the LNP of the disclosure comprises about 40 mol % ionizable lipid, about 15 mol % non-cationic helper lipid, about 45 mol % phytosterol and a structural lipid, and about 0 mol % PEG lipid. In some aspects, the LNP of the disclosure comprises about 40 mol % ionizable lipid, about 15 mol % non-cationic helper lipid, about 45 mol % phytosterol and cholesterol, and about 0 mol % PEG lipid.

In some aspects, the LNP of the disclosure comprises about 45 mol % ionizable lipid, about 15 mol % non-cationic helper lipid, about 40 mol % phytosterol, and about 0 mol % PEG lipid. In some aspects, the LNP of the disclosure comprises about 45 mol % ionizable lipid, about 15 mol % non-cationic helper lipid, about 40 mol % phytosterol and a structural lipid, and about 0 mol % PEG lipid. In some aspects, the LNP of the disclosure comprises about 45 mol % ionizable lipid, about 15 mol % non-cationic helper lipid, about 40 mol % phytosterol and cholesterol, and about 0 mol % PEG lipid.

In some aspects, the LNP of the disclosure comprises about 50 mol % ionizable lipid, about 15 mol % non-cationic helper lipid, about 35 mol % phytosterol, and about 0 mol % PEG lipid. In some aspects, the LNP of the disclosure comprises about 50 mol % ionizable lipid, about 15 mol % non-cationic helper lipid, about 35 mol % phytosterol and a structural lipid, and about 0 mol % PEG lipid. In some aspects, the LNP of the disclosure comprises about 50 mol % ionizable lipid, about 15 mol % non-cationic helper lipid, about 35 mol % phytosterol and cholesterol, and about 0 mol % PEG lipid.

In some aspects, the LNP of the disclosure comprises about 55 mol % ionizable lipid, about 15 mol % non-cationic helper lipid, about 30 mol % phytosterol, and about 0 mol % PEG lipid. In some aspects, the LNP of the disclosure comprises about 55 mol % ionizable lipid, about 15 mol % non-cationic helper lipid, about 30 mol % phytosterol and a structural lipid, and about 0 mol % PEG lipid. In some aspects, the LNP of the disclosure comprises about 55 mol % ionizable lipid, about 15 mol % non-cationic helper lipid, about 30 mol % phytosterol and cholesterol, and about 0 mol % PEG lipid.

In some aspects, the LNP of the disclosure comprises about 60 mol % ionizable lipid, about 15 mol % non-cationic helper lipid, about 25 mol % phytosterol, and about 0 mol % PEG lipid. In some aspects, the LNP of the disclosure comprises about 60 mol % ionizable lipid, about 15 mol % non-cationic helper lipid, about 25 mol % phytosterol and a structural lipid, and about 0 mol % PEG lipid. In some aspects, the LNP of the disclosure comprises about 60 mol % ionizable lipid, about 15 mol % non-cationic helper lipid, about 25 mol % phytosterol and cholesterol, and about 0 mol % PEG lipid.

In some aspects, the LNP of the disclosure comprises about 40 mol % ionizable lipid, about 10 mol % non-cationic helper lipid, about 50 mol % phytosterol, and about 0 mol % PEG lipid. In some aspects, the LNP of the disclosure comprises about 40 mol % ionizable lipid, about 10 mol % non-cationic helper lipid, about 50 mol % phytosterol and a structural lipid, and about 0 mol % PEG lipid. In some aspects, the LNP of the disclosure comprises about 40 mol % ionizable lipid, about 10 mol % non-cationic helper lipid, about 50 mol % phytosterol and cholesterol, and about 0 mol % PEG lipid.

In some aspects, the LNP of the disclosure comprises about 45 mol % ionizable lipid, about 10 mol % non-cationic helper lipid, about 45 mol % phytosterol, and about 0 mol % PEG lipid. In some aspects, the LNP of the disclosure comprises about 45 mol % ionizable lipid, about 10 mol % non-cationic helper lipid, about 45 mol % phytosterol and a structural lipid, and about 0 mol % PEG lipid. In some aspects, the LNP of the disclosure comprises about 45 mol % ionizable lipid, about 10 mol % non-cationic helper lipid, about 45 mol % phytosterol and cholesterol, and about 0 mol % PEG lipid.

In some aspects, the LNP of the disclosure comprises about 50 mol % ionizable lipid, about 0 mol % non-cationic helper lipid, about 48.5 mol % phytosterol, and about 1.5 mol % PEG lipid. In some aspects, the LNP of the disclosure comprises about 50 mol % ionizable lipid, about 0 mol % non-cationic helper lipid, about 48.5 mol % phytosterol and a structural lipid, and about 1.5 mol % PEG lipid. In some aspects, the LNP of the disclosure comprises about 50 mol % ionizable lipid, about 0 mol % non-cationic helper lipid, about 48.5 mol % phytosterol and cholesterol, and about 1.5 mol % PEG lipid.

In some aspects, the LNP of the disclosure comprises about 50 mol % ionizable lipid, about 10 mol % non-cationic helper lipid, about 40 mol % phytosterol, and about 0 mol % PEG lipid. In some aspects, the LNP of the disclosure comprises about 50 mol % ionizable lipid, about 10 mol % non-cationic helper lipid, about 40 mol % phytosterol and a structural lipid, and about 0 mol % PEG lipid. In some aspects, the LNP of the disclosure comprises about 50 mol % ionizable lipid, about 10 mol % non-cationic helper lipid, about 40 mol % phytosterol and cholesterol, and about 0 mol % PEG lipid.

In some aspects, the LNP of the disclosure comprises about 55 mol % ionizable lipid, about 10 mol % non-cationic helper lipid, about 35 mol % phytosterol, and about 0 mol % PEG lipid. In some aspects, the LNP of the disclosure comprises about 55 mol % ionizable lipid, about 10 mol % non-cationic helper lipid, about 35 mol % phytosterol and a structural lipid, and about 0 mol % PEG lipid. In some aspects, the LNP of the disclosure comprises about 55 mol % ionizable lipid, about 10 mol % non-cationic helper lipid, about 35 mol % phytosterol and cholesterol, and about 0 mol % PEG lipid.

In some aspects, the LNP of the disclosure comprises about 60 mol % ionizable lipid, about 10 mol % non-cationic helper lipid, about 30 mol % phytosterol, and about 0 mol % PEG lipid. In some aspects, the LNP of the disclosure comprises about 60 mol % ionizable lipid, about 10 mol % non-cationic helper lipid, about 30 mol % phytosterol and a structural lipid, and about 0 mol % PEG lipid. In some aspects, the LNP of the disclosure comprises about 60 mol % ionizable lipid, about 10 mol % non-cationic helper lipid, about 30 mol % phytosterol and cholesterol, and about 0 mol % PEG lipid.

In some aspects with respect to the embodiments herein, the phytosterol and a structural lipid components of a LNP of the disclosure comprises between about 10:1 and 1:10 phytosterol to structural lipid, such as about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9 and 1:10 phytosterol to structural lipid (e.g. beta-sitosterol to cholesterol).

In some embodiments, the phytosterol component of the LNP is a blend of the phytosterol and a structural lipid, such as cholesterol, wherein the phytosterol (e.g., beta-sitosterol) and the structural lipid (e.g., cholesterol) are each present at a particular mol %. For example, in some embodiments, the lipid nanoparticle comprises between 15 and 40 mol % phytosterol (e.g., beta-sitosterol). In some embodiments, the lipid nanoparticle comprises about 15, 16, 17, 18, 19, 20, 21, 22, 23, 24, 25, 26, 27, 28, 29, 30, 31, 32, 33, 34, 35, 36, 37, 38, 30 or 40 mol % phytosterol (e.g., beta-sitosterol) and 0, 1, 2, 3, 4, 5, 6, 7, 8, 9, 10, 11, 12, 13, 14, 15, 16, 17, 18, 19, 20, 21, 22, 23, 24 or 25 mol % structural lipid (e.g., cholesterol). In some embodiments, the lipid nanoparticle comprises more than 20 mol % phytosterol (e.g., beta-sitosterol) and less than 20 mol % structural lipid (e.g., cholesterol), so that the total mol % of phytosterol and structural lipid is between 30 and 40 mol %. In some embodiments, the lipid nanoparticle comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 37 mol %, about 38 mol %, about 39 mol % or about 40 mol % phytosterol (e.g., beta-sitosterol); and about 19 mol %, about 18 mol % about 17 mol %, about 16 mol %, about 15 mol %, about 14 mol %, about 13 mol %, about 12 mol %, about 11 mol %, about 10 mol %, about 9 mol %, about 8 mol %, about 7 mol %, about 6 mol %, about 5 mol %, about 4 mol %, about 3 mol %, about 2 mol %, about 1 mol % or about 0 mol %, respectively, of a structural lipid (e.g., cholesterol). In some embodiments, the lipid nanoparticle comprises about 28 mol % phytosterol (e.g., beta-sitosterol) and about 10 mol % structural lipid (e.g., cholesterol). In some embodiments, the lipid nanoparticle comprises a total mol % of phytosterol and structural lipid (e.g., cholesterol) of 38.5%. In some embodiments, the lipid nanoparticle comprises 28.5 mol % phytosterol (e.g., beta-sitosterol) and 10 mol % structural lipid (e.g., cholesterol). In some embodiments, the lipid nanoparticle comprises 18.5 mol % phytosterol (e.g., beta-sitosterol) and 20 mol % structural lipid (e.g., cholesterol).

Lipid nanoparticles of the disclosure may be designed for one or more specific applications or targets. For example, the subject lipid nanoparticles may optionally be designed to further enhance delivery of a nucleic acid molecule, such as an RNA, to a particular immune cell (e.g., lymphoid cell or myeloid cell), tissue, organ, or system or group thereof in a mammal's, e.g., a human's body. Physiochemical properties of lipid nanoparticles may be altered in order to increase selectivity for particular bodily targets. For instance, particle sizes may be adjusted to promote immune cell uptake. As set forth above, the nucleic acid molecule included in a lipid nanoparticle may also be selected based on the desired delivery to immune cells. For example, a nucleic acid molecule may be selected for a particular indication, condition, disease, or disorder and/or for delivery to a particular cell, tissue, organ, or system or group thereof (e.g., localized or specific delivery).

In certain embodiments, a lipid nanoparticle may include an mRNA encoding a polypeptide of interest capable of being translated within a cell to produce a polypeptide of interest. In other embodiments, the lipid nanoparticle can include other types of agents, such as other nucleic acid agents, including DNA and/or RNA agents, as described herein, e.g., siRNAs, miRNAs, antisense nucleic acid and the like as described in further detail below.

The amount of a nucleic acid molecule in a lipid nanoparticle may depend on the size, composition, desired target and/or application, or other properties of the lipid nanoparticle as well as on the properties of the therapeutic and/or prophylactic. For example, the amount of an RNA useful in a lipid nanoparticle may depend on the size, sequence, and other characteristics of the RNA. The relative amounts of a nucleic acid molecule and other elements (e.g., lipids) in a lipid nanoparticle may also vary. In some embodiments, the wt/wt ratio of the ionizable lipid component to a a nucleic acid molecule, in a lipid nanoparticle may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the ionizable lipid component to a nucleic acid molecule may be from about 10:1 to about 40:1. In certain embodiments, the wt/wt ratio is about 20:1. The amount of a nucleic acid molecule in a LNP may, for example, be measured using absorption spectroscopy (e.g., ultraviolet-visible spectroscopy).

In some embodiments, a lipid nanoparticle includes one or more RNAs, and one or more ionizable lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 5.7:1, about 5.8:1, about 5.9:1, about 6.0:1, about 6.5:1, or about 7.0:1. For example, the N:P ratio may be about 5.67:1. In another embodiment, the N:P ratio may be about 5.8:1.

In some embodiments, the formulation including a lipid nanoparticle may further includes a salt, such as a chloride salt.

In some embodiments, the formulation including a lipid nanoparticle may further includes a sugar such as a disaccharide. In some embodiments, the formulation further includes a sugar but not a salt, such as a chloride salt.

### Physical Properties

The characteristics of a lipid nanoparticle may depend on the components thereof. For example, a lipid nanoparticle including cholesterol as a structural lipid may have different characteristics than a lipid nanoparticle that includes a different structural lipid. Similarly, the characteristics of a lipid nanoparticle may depend on the absolute or relative amounts of its components. For instance, a lipid nanoparticle including a higher molar fraction of a phospholipid may have different characteristics than a lipid nanoparticle including a lower molar fraction of a phospholipid. Characteristics may also vary depending on the method and conditions of preparation of the lipid nanoparticle.

Lipid nanoparticles may be characterized by a variety of methods. For example, microscopy (e.g., transmission electron microscopy or scanning electron microscopy) may be used to examine the morphology and size distribution of a lipid nanoparticle. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a lipid nanoparticle, such as particle size, polydispersity index, and zeta potential.

The mean size of a lipid nanoparticle may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a lipid nanoparticle may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a lipid nanoparticle may be from about 70 nm to about 100 nm. In a particular embodiment, the mean size may be about 80 nm. In other embodiments, the mean size may be about 100 nm.

A lipid nanoparticle may be relatively homogenous. A polydispersity index may be used to indicate the homogeneity of a LNP, e.g., the particle size distribution of the lipid nanoparticles. As used herein, the “polydispersity index” is a ratio that describes the homogeneity of the particle size distribution of a system. A small value, e.g., less than 0.3, indicates a narrow particle size distribution. A small (e.g., less than 0.3) polydispersity index generally indicates a narrow particle size distribution. A lipid nanoparticle may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a lipid nanoparticle may be from about 0.10 to about 0.20.

The zeta potential of a lipid nanoparticle may be used to indicate the electrokinetic potential of the composition. As used herein, the “zeta potential” is the electrokinetic potential of a lipid, e.g., in a particle composition.

For example, the zeta potential may describe the surface charge of a lipid nanoparticle. Lipid nanoparticles with relatively low charges, positive or negative, are generally desirable, as more highly charged species may interact undesirably with cells, tissues, and other elements in the body. In some embodiments, the zeta potential of a lipid nanoparticle may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a a nucleic acid molecule describes the amount of nucleic acid molecule that is encapsulated or otherwise associated with a lipid nanoparticle after preparation, relative to the initial amount provided. The encapsulation efficiency is desirably high (e.g., close to 100%). The encapsulation efficiency may be measured, for example, by comparing the amount of nucleic acid molecule in a solution containing the lipid nanoparticle before and after breaking up the lipid nanoparticle with one or more organic solvents or detergents. Fluorescence may be used to measure the amount of free nucleic acid molecules (e.g., RNA) in a solution. For the lipid nanoparticles described herein, the encapsulation efficiency of a nucleic acid molecule may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A lipid nanoparticle may optionally comprise one or more coatings. For example, a lipid nanoparticle may be formulated in a capsule, film, or tablet having a coating. A capsule, film, or tablet including a composition described herein may have any useful size, tensile strength, hardness, or density.

### Pharmaceutical Compositions

Formulations comprising lipid nanoparticles of the invention may be formulated in whole or in part as pharmaceutical compositions. Pharmaceutical compositions may include one or more lipid nanoparticles. For example, a pharmaceutical composition may include one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a LNP in the formulation of the disclosure. An excipient or accessory ingredient may be incompatible with a component of a LNP of the formulation if its combination with the component or LNP may result in any undesirable biological effect or otherwise deleterious effect.

A lipid nanoparticle of the disclosure formulated into a pharmaceutical composition can encapsulate a single nucleic acid or multiple nucleic acids. When encapsulating multiple nucleic acids, the nucleic acids can be of the same type (e.g., all siRNAs) or can be of different types (e.g., siRNAs and other RNA interference agents, such as miRNAs, or siRNAs and mRNAs). Furthermore, multiple LNPs can be formulated into the same or separate pharmaceutical compositions. For example, the same or separate pharmaceutical compositions can comprise a first LNP and a second LNP, wherein the first and second LNP encapsulate the same or different nucleic acid molecules, wherein the first and second LNP include an immune cell delivery potentiating lipid as a component. In other embodiments, the same or separate pharmaceutical compositions can comprise a first LNP and a second LNP, wherein the first and second LNP encapsulate the same or different nucleic acid molecules, wherein the first LNP includes an immune cell delivery potentiating lipid as a component and the second LNP lacks an immune cell delivery potentiating lipid.

In some embodiments, one or more excipients or accessory ingredients may make up greater than 50% of the total mass or volume of a pharmaceutical composition including an LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

Lipid nanoparticles and/or pharmaceutical compositions including one or more lipid nanoparticles may be administered to any patient or subject, including those patients or subjects that may benefit from a therapeutic effect provided by the delivery of a therapeutic and/or prophylactic to one or more particular cells, tissues, organs, or systems or groups thereof, such as the renal system. Although the descriptions provided herein of lipid nanoparticles and pharmaceutical compositions including lipid nanoparticles are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more lipid nanoparticles may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., lipid nanoparticle). The amount of the active ingredient is generally equal to the dosage of the active ingredient which would be administered to a subject and/or a convenient fraction of such a dosage such as, for example, one-half or one-third of such a dosage.

Pharmaceutical compositions may be prepared in a variety of forms suitable for a variety of routes and methods of administration. In one embodiment, such compositions are prepared in liquid form or are lyophylized (e.g., and stored at 4° C. or below freezing). For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 m to 500 m. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### Uses of Lipid-Based Compositions

The present disclosure provides improved lipid-based compositions, in particular LNP compositions, with enhanced delivery of RNA interference agents (e.g., siRNAs) to immune cells. The present disclosure is based, at least in part, on the discovery that components of LNPs act as immune cell delivery potentiating lipids that enhance delivery of an encapsulated RNA interfering agents (e.g., siRNA) to immune cells such that expression of the protein encoded by the mRNA targeted by the RNA interfering agent is inhabited in the immune cells. Furthermore, inhibition of expression of the protein encoded by the mRNA targeted by the RNA interfering agent modulates the activity of the immune cell, such as modulating differentiation of the immune cell and/or modulation of the effector function(s) of the immune cell.

The improved lipid-based compositions of the disclosure, in particular LNPs, are useful for a variety of purposes, both in vitro and in vivo, such as for delivery of RNA interference agents to immune cells, modulating immune cell (e.g., T cell, B cell, NK cell, dendritic cell, myeloid cell or macrophage) activation or activity and modulation of immune cell responses, including upregulation of immune responses (e.g., to enhance immunity in the treatment of cancer or infectious diseases) and downregulation of immune responses (e.g., to reduce autoimmunity in autoimmune and inflammatory disorders).

For in vitro delivery of the RNA interference agent (e.g., siRNA), the immune cell is contacted with the LNP by incubating the LNP and the immune cell ex vivo. Such immune cells may subsequently be introduced in vivo by administering the cells to a subject.

For in vivo delivery of RNA interference agents (e.g., siRNAs), the immune cell is contacted with the LNP by administering the LNP to a subject to thereby increase or induce expression of the RNA interference agent in the immune cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

For in vitro delivery, in one embodiment the immune cell is contacted with the LNP by incubating the LNP and the immune cell ex vivo. In one embodiment, the immune cell is a human immune cell. In another embodiment, the immune cell is a primate immune cell. In another embodiment, the immune cell is a human or non-human primate immune cell. In one embodiment, the immune cell is a T cell (e.g., a CD3+ T cell, a CD4+ T cell, a CD8+ T cell, a CD4+CD25+CD127^{low} Treg cell or a Th17 cell). In one embodiment, the immune cell is a B cell (e.g., a CD19+ B cell). In one embodiment, the immune cell is a dendritic cell (e.g., a CD11c+CD11b− dendritic cell). In one embodiment, the immune cell is a monocyte/macrophage (e.g., a CD11c−CD11b+ monocyte/macrophage). In one embodiment, the immune cell is an immature NK cell (e.g., a CD56^{HIGH} immature NK cell). In one embodiment, the immune cell is an activated NK cell (e.g., a CD56^{DIM} activated NK cell). In one embodiment, the immune cell is an NK T cell (e.g., a CD3+CD56+ NK T cell

In one embodiment, the immune cell is contacted with the LNP in the presence of serum or C1q for at least 15 minutes, which has been shown to be sufficient time for transfection of the cells ex vivo. In another embodiment, the immune cell is contacted with the LNP for, e.g., at least 30 minutes, at least 1 hour, at least 2 hours, at least 3 hours, at least 4 hours, at least 5 hours, at least 6 hours, at least 12 hours or at least 24 hours.

In one embodiment, the immune cell is contacted with the LNP for a single treatment/transfection. In another embodiment, the immune cell is contacted with the LNP for multiple treatments/transfections (e.g., two, three, four or more treatments/transfections of the same cells). Repeat transfection of the same cells has been demonstrated to lead to a dose-related increase in the percentage of cells transfected and in the level of expression of a protein encoded by the transfected nucleic acid without impacting cell viability.

In another embodiment, for in vivo delivery, the immune cell is contacted with the LNP by administering the LNP to a subject to thereby deliver the nucleic acid to immune cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

In one embodiment, an intracellular concentration of the RNA interference agent in the immune cell is enhanced. In one embodiment, an activity of the RNA interference agent in the immune cell is enhanced. In one embodiment, expression of the RNA interference agent in the immune cell is enhanced. In on embodiment, the RNA interference agent modulates the activation or activity of the immune cell. In one embodiment, the RNA interference agent decreases the activation or activity of the immune cell. In another embodiment, the RNA interference agent increases the activation or activity of the immune cell.

In certain embodiments, delivery of a nucleic acid to an immune cell by the immune cell delivery potentiating lipid-containing LNP results in delivery to a detectable amount of immune cells (e.g., delivery to a certain percentage of immune cells), e.g., in vivo following administration to a subject. In some embodiments, the immune cell delivery potentiating lipid containing LNP does not include a targeting moiety for immune cells (e.g., does not include an antibody with specificity for an immune cell marker, or a receptor ligand which targets the LNP to immune cells). For example, in one embodiment, administration of the immune cell delivery potentiating lipid-containing LNP results in delivery of the nucleic acid to at least about 15% of splenic T cells in vivo after a single intravenous injection. In another embodiment, administration of the immune cell delivery potentiating lipid-containing LNP results in delivery of the nucleic acid to at least about 15%-25% of splenic B cells in vivo after a single intravenous injection. In another embodiment, administration of the immune cell delivery potentiating lipid-containing LNP results in delivery of the nucleic acid to at least about 35%-40% of splenic dendritic cells in vivo after a single intravenous injection. In another embodiment, administration of the immune cell delivery potentiating lipid-containing LNP results in delivery of the nucleic acid to at least about 5%-20% of bone marrow cells (femur and/or humerus) in vivo after a single intravenous injection. The levels of delivery demonstrated herein make in vivo immune therapy possible.

In one embodiment, uptake of the RNA interference agent by the immune cell is enhanced. Uptake can be determined by methods known to one of skill in the art. For example, association/binding and/or uptake/internalization may be assessed using a detectably labeled, such as fluorescently labeled, LNP and tracking the location of such LNP in or on immune cells following various periods of incubation. In addition, mathematical models, such as the ordinary differential equation (ODE)-based model described by Radu Mihaila, et al., (Molecular Therapy: Nucleic Acids, Vol. 7: 246-255, 2017; herein incorporated by reference), allow for quantitation of delivery and uptake.

In another embodiment, function or activity of the RNA interference agent can be used as an indication of the delivery of the agent to the immune cell. For example, a decrease in protein expression in a certain proportion of immune cells can be measured to indicate delivery of the RNA interference agent (e.g., siRNA) that targets the mRNA encoding the protein to that proportion of cells. Decreases in protein expression can be measured by assays readily available in the art, such as described in the Examples.

In one embodiment, various agents can be used to label cells (e.g., T cell, B cell, NK cells, dendritic cells, myeloid cells, macrophages) to measure delivery to that specific immune cell population. For example, the LNP can also encapsulate a reporter nucleic acid (e.g., an mRNA encoding a detectable reporter protein), wherein expression of the reporter nucleic acid results in labeling of the cell population to which the reporter nucleic acid is delivered. Non-limiting examples of detectable reporter proteins include enhanced green fluorescent protein (EGFP) and luciferase.

Delivery of the RNA interference agent (e.g., siRNA) to the immune cell by the immune cell delivery potentiating lipid-containing LNP can be measured in vitro or in vivo by, for example, detecting decreased expression of a protein encoded by the mRNA targeted by the RNA interference agent (i.e., knock down of protein expression) or by detecting an effect (e.g., a biological effect) mediated by the RNA interference agent associated with/encapsulated by the LNP. For detection of protein knock down, the protein can be assayed by, for example, immunofluorescence or flow cytometery using an antibody that specifically binds the protein.

Methods of the disclosure are useful to deliver RNA interference agents to a variety of immune cell types. In one embodiment, the immune cell is selected from the group consisting of T cells, B cells, NK cells, dendritic cells, myeloid cells and macrophages.

The methods can be used to deliver RNA interference agents to immune cells located, for example, in the spleen, in the peripheral blood and/or in the bone marrow. In one embodiment, the immune cell is a T cell. T cells can be identified by expression of one or more T cell markers known in the art, typically CD3. Additional T cell markers include CD4 or CD8. In one embodiment, the immune cell is a B cell. B cells can be identified by expression of one or more B cell markers known in the art, typically CD19. Additional B cell markers include CD24 and CD72. In one embodiment, the immune cell is a monocyte and/or a tissue macrophage. Monocytes and/or macrophages can be identified by expression of one or more monocyte and/or macrophage markers known in the art, such as CD2, CD11b, CD14 and/or CD16. In one embodiment, the immune cell is a dendritic cell. Dendritic cells can be identified by expression of one or more dendritic cell markers known in the art, typically CD11c. Additional dendritic cell markers include BDCA-1 and/or CD103.

The improved lipid-based compositions, including LNPs of the disclosure are useful to deliver more than one nucleic acid molecules (wherein at least one of the nucleic acid molecules is an RNA interference agent) to an immune cell or different populations of immune cells, by for example, administration of two or more different LNPs. In one embodiment, the method of the disclosure comprises contacting the immune cell (or administering to a subject), concurrently or consecutively, a first LNP and a second LNP, wherein the first and second LNP encapsulate the same or different nucleic acid molecules (e.g., the same or different RNA interference agents, such as the same or different siRNAs), wherein the first and second LNP include a phytosterol as a component. In other embodiments, the method of the disclosure comprises contacting the immune cell (or administering to a subject), concurrently or consecutively, a first LNP and a second LNP, wherein the first and second LNP encapsulate the same or different nucleic acid molecules (e.g., the same or different RNA interference agents, such as the same or different siRNAs), wherein the first LNP includes a phytosterol as a component and the second LNP lacks a phytosterol.

### Methods of Modulating Immune Cell Activity

The disclosure provides a method for modulating immune cell activity (e.g., T cell activity, B cell activity, NK cell activity, dendritic cell activity, myeloid cell activity and/or macrophage activity). To enhance delivery into an immune cell, RNA interference agents of the disclosure are administered to the immune cell or to a subject encapsulated in a lipid nanoparticle that comprises at least one immune cell delivery potentiating lipid, as described herein.

In one embodiment, immune cell activity is modulated in vitro. In one embodiment, immune cell activity is stimulated in vitro. In one embodiment, immune cell activity is inhibited in vitro. In another embodiment, immune cell activity is modulated in vivo, e.g., in a subject, such as a human subject. In one embodiment, immune cell activity is stimulated in vivo. In another embodiment, immune cell activity is inhibited in vivo. In one embodiment, the method comprises administering to the immune cell (e.g., administering to a subject) a composition of the disclosure (or lipid nanoparticle thereof, or pharmaceutical composition thereof) comprising at least one RNA interference agent (e.g., siRNA), such that activity of the immune cell is modulated. In one embodiment, modulating immune cell activity comprises modulating immune cell proliferation. In one embodiment, modulating immune cell activity comprises modulating cytokine production. In one embodiment, modulating immune cell activity comprises modulating immune cell effector function, such as modulating T helper or Treg functions or modulating immunoglobulin production by B cells, e.g., antigen-specific antibody production.

Modulation of immune cell activity, either in vitro or in a subject, can be evaluated by a variety of methods established in the art for assessing immune responses, including but not limited to the methods described in the Examples. For example, in various embodiments, modulation is evaluated by measuring levels of cytokine production and/or antibody production, such as by standard ELISA, and/or by evaluating cell proliferation by standard methods known in the art.

LNP compositions of the disclosure are administered to a subject at an effective amount. In general, an effective amount of the LNP composition will allow for efficient expression of the RNA interference agent in the immune cell such that expression of the protein encoded by the mRNA targeted by the RNA interference agent is inhibited. Metrics for efficiency may include polypeptide translation (indicated by polypeptide expression), level of mRNA degradation, and immune response indicators.

### Therapeutic Methods

The methods of the disclosure for modulating immune cell activity in a subject can be used in a variety of clinical, prophylactic or therapeutic applications. In one embodiment, modulating immune cell activity comprises stimulating immune cell activity in the subject. In another embodiment, modulating immune cell activity comprises inhibiting immune cell activity in the subject. Accordingly, the disclosure provides a method of modulating (e.g., stimulating or inhibiting) an immune response in a subject, the method comprising administering to the subject an LNP composition of the disclosure such that an immune response is modulated (e.g. stimulated or inhibited) in the subject.

Stimulation of Immune Responses

To stimulate (e.g., enhance) an immune response, the target immune cell(s) and RNA interference agent (e.g., siRNA) are selected such that knock down of the protein encoded by the mRNA targeted by the RNA interference agent results in either: (i) stimulation of an immune cell that is a positive regulator (i.e., upregulator) of immune responses, resulting in an effect(s) such as increased cell differentiation, increased cell proliferation and/or increased cell effector function of the positive regulator of immune responses, thereby enhancing immune responses; or (ii) inhibition of an immune cell that is a negative regulator (i.e., downregulator) of immune responses, resulting in an effect(s) such as decreased cell differentiation, decreased cell proliferation and/or decreased cell effector function of the negative regulator of immune responses, thereby enhancing immune responses.

In one embodiment for stimulating an immune response, the target immune cell is a Treg cell and the RNA interference agent is an siRNA that targets Foxp3 mRNA. Knock down of Foxp3 in the Treg cells by the siRNA leads to decreased Treg cell differentiation (see Example 4) and decreased Treg cell suppressor function (see Example 5). Thus, by downregulating Treg cell activity by delivery of the Foxp3 siRNA into the cells, the negative regulatory function of the Treg cells is inhibited, thereby allowing for enhanced immune responses.

In one embodiment, the method for stimulating an immune response is used with a subject suffering from cancer (i.e., a tumor bearing subject), to thereby enhance an immune response against the cancer in the subject. Non-limiting examples of cancers that can be treated include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In particular embodiments, the cancer is liver cancer (e.g., hepatocellular carcinoma) or colorectal cancer. In other embodiments, the cancer is a blood-based cancer or a hematopoetic cancer.

In another embodiment, the method for stimulating an immune response is used with a subject suffering from an infectious disease, to thereby enhance an immune response against the infectious disease pathogen in the subject. Non-limiting examples of infectious diseases that can be treated include those caused by viral, bacterial, funcal, yeast and parasitic pathogens.

In yet another embodiment, the method for stimulating an immune response is used with a subject that is receiving or has received a vaccine, to thereby enhance an immune response against the vaccine antigen(s). An LNP composition of the invention can be administered prior to, concurrent with, or subsequent to the vaccine administration to the subject.

Accordingly, in one aspect, the disclosure pertains to a method of stimulating an immune response in a subject in need thereof, the method comprising administering to the subject a composition of the disclosure (or lipid nanoparticle thereof, or pharmaceutical composition thereof). The method can further comprise administering one or more additional agents to the subject, such as one or more additional immunostimulatory agents (non-limiting examples of which include immune checkpoint inhibitors, such as anti-PD-1, anti-PD-L1, anti-PD-L2 and/or anti-CTLA-4). In some embodiments, the LNP, or pharmaceutical composition, is administered to the patient parenterally. In particular embodiments, the subject is a mammal, e.g., a human. In various embodiments, the subject is provided with an effective amount of the LNP composition.

Inhibition of Immune Responses

To inhibit (e.g., decrease) an immune response, the target immune cell(s) and RNA interference agent (e.g., siRNA) are selected such that knock down of the protein encoded by the mRNA targeted by the RNA interference agent results in either: (i) inhibition of an immune cell that is a positive regulator (i.e., upregulator) of immune responses, resulting in an effect(s) such as decreased cell differentiation, decreased cell proliferation and/or decreased cell effector function of the positive regulator of immune responses, thereby inhibiting immune responses; or (ii) stimulation of an immune cell that is a negative regulator (i.e., downregulator) of immune responses, resulting in an effect(s) such as increased cell differentiation, increased cell proliferation and/or increased cell effector function of the negative regulator of immune responses, thereby inhibiting immune responses.

In one embodiment for inhibiting an immune response, the target immune cell is a Th17 cell and the RNA interference agent is an siRNA that targets ROR mRNA (e.g., RORc, RORγt and/or RORα). In another embodiment for inhibiting an immune response, the target immune cell is a Th17 cell and the RNA interference agent is an siRNA that targets IL-17a mRNA. Knock down of ROR and/or IL-17a in the Th17 cells by the siRNA leads to decreased cytokine production (e.g., IL-17 production) by the Th17 cells (see Example 6), thereby inhibiting their functional activity. Thus, by downregulating Th17 cell activity by delivery of the ROR and/or IL-17a siRNA into the cells, the positive regulatory functions of the Th17 cells are inhibited, thereby allowing for inhibition of immune responses.

In one embodiment, the method for inhibiting an immune response is used with a subject having aberrant immune activity, including subjects suffering from an autoimmune disease, an allergic disorder or an inflammatory response. In another embodiment, the subject is suspected of having an autoimmune disorder. In another embodiment, the subject is at risk of developing an autoimmune disorder. Non-limiting examples of autoimmune diseases that can be treated according to the method of the disclosure include rheumatoid arthritis, systemic lupus erythematosus, inflammatory bowel disease (including ulcerative colitis and Crohn's disease), Type 1 diabetes, multiple sclerosis, psoriasis, Graves' disease, Hashimoto's thyroiditis, chronic inflammatory demyelinating polyneuropathy, Guillain-Barre syndrome, myasthenia gravis, glomerulonephritis and vasculitis.

Furthermore, the methods for inhibiting an immune response can be used to inhibit transplant rejection in organ transplant recipients and inhibit graft-versus-host disease, e.g., in bone marrow transplant recipients. Still further, the methods can be used to downregulate immune cell activity in immunotherapy regimens, to thereby provide control of the degree of immune activation that is stimulated for therapeutic purposes. In particular, in situations where an immunotherapy regimen results in overstimulation of immune responses and detrimental side effects therefrom, the immunoinhibitory methods of the disclosure can be used to “tamp down” the degree of immunostimulation provided by the immunotherapy regimen to thereby lessen detrimental side effects therefrom. Non-limiting examples of clinical immunotherapy regimens that can be modulated according to the methods of the invention include treatment with immune checkpoint inhibitors (e.g., agents that target CTLA4, PD-1 or PD-L1) and treatment with CAR-T cells (adoptive T cell transfer immunotherapies).

Accordingly, in one aspect, the disclosure pertains to a method of inhibiting an immune response in a subject in need thereof, the method comprising administering to the subject a composition of the disclosure (or lipid nanoparticle thereof, or pharmaceutical composition thereof). The method can further comprise administering one or more additional agents to the subject, such as one or more additional immunoinhibitory or immunosuppressive agents. In some embodiments, the LNP, or pharmaceutical composition, is administered to the patient parenterally. In particular embodiments, the subject is a mammal, e.g., a human. In various embodiments, the subject is provided with an effective amount of the LNP composition.

Treatment Regimens

A pharmaceutical composition including one or more RNA interference agents (e.g., siRNAs) of the disclosure may be administered to a subject by any suitable route. In some embodiments, compositions of the disclosure are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. In some embodiments, a composition is administered intramuscularly. However, the present disclosure encompasses the delivery of compositions of the disclosure by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In certain embodiments, compositions of the disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of RNA interference agent or nanoparticle per 1 kg of subject body weight. In particular embodiments, a dose of about 0.005 mg/kg to about 5 mg/kg of RNA interference agent or nanoparticle of the disclosure may be administrated.

In some embodiments the dosage of the RNA interference agent in the therapeutic composition is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 100-300 μg, 120-250 μg, 150-250 μg, 180-280 μg, 200-300 μg, 30-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the immunomodulatory therapeutic composition is administered to the subject by intradermal or intramuscular injection. In some embodiments, the immunomodulatory therapeutic composition is administered to the subject on day zero. In some embodiments, a second dose of the immunomodulatory therapeutic composition is administered to the subject on day seven, or day fourteen or day twenty one.

In some embodiments, a dosage of 25 micrograms of the RNA interference agent is included in the immunomodulatory therapeutic composition administered to the subject. In some embodiments, a dosage of 10 micrograms of the RNA interference agent is included in the immunomodulatory therapeutic composition administered to the subject. In some embodiments, a dosage of 30 micrograms of the RNA interference agent is included in the immunomodulatory therapeutic composition administered to the subject. In some embodiments, a dosage of 100 micrograms of the RNA interference agent is included in the immunomodulatory therapeutic composition administered to the subject. In some embodiments, a dosage of 50 micrograms of the RNA interference agent is included in the immunomodulatory therapeutic composition administered to the subject. In some embodiments, a dosage of 75 micrograms of the RNA interference agent is included in the immunomodulatory therapeutic composition administered to the subject. In some embodiments, a dosage of 150 micrograms of the RNA interference agent is included in the immunomodulatory therapeutic composition administered to the subject. In some embodiments, a dosage of 400 micrograms of the RNA interference agent is included in the immunomodulatory therapeutic composition administered to the subject. In some embodiments, a dosage of 300 micrograms of the RNA interference agent is included in the immunomodulatory therapeutic composition administered to the subject. In some embodiments, a dosage of 200 micrograms of the RNA interference agent is included in the immunomodulatory therapeutic composition administered to the subject. In other embodiments the immunomodulatory therapeutic composition is chemically modified and in other embodiments the immunomodulatory therapeutic composition is not chemically modified.

In some embodiments, the effective amount is a total dose of 1-100 μg. In some embodiments, the effective amount is a total dose of 100 μg. In some embodiments, the effective amount is a dose of 25 μg administered to the subject a total of one or two times. In some embodiments, the effective amount is a dose of 100 μg administered to the subject a total of two times. In some embodiments, the effective amount is a dose of 1 μg-10 μg, 1 μg-20 μg, 1 μg-30 μg, 5 μg-10 μg, 5 μg-20 μg, 5 μg-30 μg, 5 μg-40 μg, 5 μg-50 μg, 10 μg-15 μg, 10 μg-20 μg, 10 μg-25 μg, 10 μg-30 μg, 10 μg-40 μg, 10 μg-50 μg, 10 μg-60 μg, 15 μg-20 μg, 15 μg-25 μg, 15 μg-30 μg, 15 μg-40 μg, 15 μg-50 μg, 20 μg-25 μg, 20 μg-30 μg, 20 μg-40 μg 20 μg-50 μg, 20 μg-60 μg, 20 μg-70 μg, 20 μg-75 μg, 30 μg-35 μg, 30 μg-40 μg, 30 μg-45 μg 30 μg-50 μg, 30 μg-60 μg, 30 μg-70 μg, 30 μg-75 μg which may be administered to the subject a total of one or two times or more.

A dose may be administered one or more times per day, in the same or a different amount, to obtain a desired level of RNA interference agent (e.g., siRNA) expression and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). For example, in certain embodiments, a composition of the disclosure is administered at least two times wherein the second dose is administered at least one day, or at least 3 days, or least 7 days, or at least 10 days, or at least 14 days, or at least 21 days, or at least 28 days, or at least 35 days, or at least 42 days or at least 48 days after the first dose is administered. In certain embodiments, a first and second dose are administered on days 0 and 2, respectively, or on days 0 and 7 respectively, or on days 0 and 14, respectively, or on days 0 and 21, respectively, or on days 0 and 48, respectively. Additional doses (i.e., third doses, fourth doses, etc.) can be administered on the same or a different schedule on which the first two doses were administered. For example, in some embodiments, the first and second dosages are administered 7 days apart and then one or more additional doses are administered weekly thereafter. In another embodiment, the first and second dosages are administered 7 days apart and then one or more additional doses are administered every two weeks thereafter.

In some embodiments, a single dose may be administered, for example, prior to or after a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more RNA interference agents employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

In some embodiments, a pharmaceutical composition of the disclosure may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure, or imaging, diagnostic, or prophylactic compositions thereof in combination with agents that improve their bioavailability, reduce and/or modify their metabolism, inhibit their excretion, and/or modify their distribution within the body.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

In any of the foregoing or related aspects, the disclosure provides a kit comprising a container comprising a lipid nanoparticle, and an optional pharmaceutically acceptable carrier, or a pharmaceutical composition, and a package insert comprising instructions for administration of the lipid nanoparticle or pharmaceutical composition for modulating an immune response in an individual.

In any of the foregoing or related aspects, the disclosure provides a kit comprising a medicament comprising a lipid nanoparticle, and an optional pharmaceutically acceptable carrier, or a pharmaceutical composition, and a package insert comprising instructions for administration of the medicament for modulating an immune response in an individual.

### Definitions

An “autoimmune disorder,” as used herein, refers to a disease state in which, via the action of white blood cells (e.g., B cells, T cells, macrophages, monocytes, or dendritic cells), a pathological immune response (e.g., pathological in duration and/or magnitude) against one or more endogenous antigens, i.e., one or more autoantigens, with consequent tissue damage that may result from direct attack on the cells bearing the one or more autoantigens, from immune-complex formation, or from local inflammation. Autoimmune diseases are characterized by increased inflammation due to immune system activation against self-antigens.

The terms “allograft”, “homograft” and “allogeneic graft” refer to the transplant of an organ or tissue from one individual to another of the same species with a different genotype, including transplants from cadaveric, living related, and living unrelated donors. A graft transplanted from one individual to the same individual is referred to as an “autologous graft” or “autograft”. A graft transplanted between two genetically identical or syngeneic individuals is referred to as a “syngeneic graft”. A graft transplanted between individuals of different species is referred to as a “xenogeneic graft” or “xenograft”.

As used herein the phrase “immune response” or its equivalent “immunological response” refers to the development of a cellular (mediated by antigen-specific T cells or their secretion products) directed against an autoantigen or an related epitope of an autoantigen. A cellular immune response is elicited by the presentation of polypeptide epitopes in association with Class I or Class II MHC molecules, to activate antigen-specific CD4+ T helper cells and/or CD8+ cytotoxic T cells. The response may also involve activation of other components.

As used herein, the term “immune cell” refers to cells that play a role in the immune response, including lymphocytes, such as B cells and T cells; natural killer cells; dendritic cells, myeloid cells, such as monocytes, macrophages, eosinophils, mast cells, basophils, and granulocytes.

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

“Immunotherapy” refers to the treatment of a subject afflicted with, or at risk of contracting or suffering a recurrence of, a disease by a method comprising inducing, enhancing, suppressing or otherwise modifying an immune response.

A human “at risk of developing an autoimmune disorder” refers to a human with a family history of autoimmune disorders (e.g., a genetic predisposition to one or more inflammatory disorders) or one exposed to one or more autoimmune disorder/autoantibody-inducing conditions. For example, a human exposed to a shiga toxin is at risk for developing typical HUS. Humans with certain cancers (e.g., liquid tumors such as multiple myeloma or chronic lymphocytic leukemia) can pre-dispose patients to developing certain autoimmune hemolytic diseases. For example, PCH can follow a variety of infections (e.g., syphilis) or neoplasms such as non-Hodgkin's lymphoma. In another example, CAD can be associated with HIV infection, Mycoplasma pneumonia infection, non-Hodgkin's lymphoma, or Waldenstrom's macroglobulinemia. In yet another example, autoimmune hemolytic anemia is a well-known complication of human chronic lymphocytic leukemia, approximately 11% of CLL patients with advanced disease will develop AIHA. As many as 30% of CLL may be at risk for developing AIHA. See, e.g., Diehl et al. (1998) Semin Oncol 25(1):80-97 and Gupta et al. (2002) Leukemia 16(10):2092-2095.

A human “suspected of having an autoimmune disorder” is one who presents with one or more symptoms of an autoimmune disorder. Symptoms of autoimmune disorders can vary in severity and type with the particular autoimmune disorder and include, but are not limited to, redness, swelling (e.g., swollen joints), joints that are warm to the touch, joint pain, stiffness, loss of joint function, fever, chills, fatigue, loss of energy, pain, fever, pallor, icterus, urticarial dermal eruption, hemoglobinuria, hemoglobinemia, and anemia (e.g., severe anemia), headaches, loss of appetite, muscle stiffness, insomnia, itchiness, stuffy nose, sneezing, coughing, one or more neurologic symptoms such as dizziness, seizures, or pain. From the above it will be clear that not all humans are “suspected of having an autoimmune disorder.”

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a LNP, “about” may mean +/−5% of the recited value. For instance, a LNP including a lipid component having about 40% of a given compound may include 30-50% of the compound. In another example, delivery to at least about 15% of T cells may include delivery to 10-20% of T cells.

Cancer: As used herein, “cancer” is a condition involving abnormal and/or unregulated cell growth, e.g., a cell having deregulated control of G1 progression. Exemplary non-limiting cancers include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In particular embodiments, the cancer is liver cancer (e.g., hepatocellular carcinoma) or colorectal cancer. In other embodiments, the cancer is a blood-based cancer or a hematopoetic cancer.

Contacting: As used herein, the term “contacting” means establishing a physical connection between two or more entities. For example, contacting a cell with a lipid nanoparticle composition means that the cell and lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering a LNP including the therapeutic and/or prophylactic to the subject (e.g., by an intravenous, intramuscular, intradermal, or subcutaneous route). Administration of a LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an siRNA), or other composition may be fully encapsulated, partially encapsulated, or substantially encapsulated. For example, in some embodiments, an siRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of a LNP, relative to the initial total amount of therapeutic and/or prophylactic used in the preparation of a LNP. For example, if 97 mg of therapeutic and/or prophylactic are encapsulated in a LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Enhanced delivery: As used herein, the term “enhanced delivery” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a nucleic acid (e.g., a therapeutic and/or prophylactic mRNA) by a nanoparticle to a target cell of interest (e.g., immune cell) compared to the level of delivery of the nucleic acid (e.g., a therapeutic and/or prophylactic mRNA) by a control nanoparticle to a target cell of interest (e.g., immune cell). For example, “enhanced delivery” by a immune cell delivery potentiating lipid-containing LNP of the disclosure can be evaluated by comparison to the same LNP lacking an immune cell delivery potentiating lipid. The level of delivery of an immune cell delivery potentiating lipid-containing LNP to a particular cell (e.g., immune cell) may be measured by comparing the amount of protein produced in target cells using the phytosterol-containing LNP versus the same LNP lacking the immune cell delivery potentiating lipid (e.g., by mean fluorescence intensity using flow cytometry), comparing the % of target cells transfected using the immune cell delivery potentiating lipid-containing LNP versus the same LNP lacking the immune cell delivery potentiating lipid (e.g., by quantitative flow cytometry), or comparing the amount of therapeutic and/or prophylactic in target cells in vivo using the immune cell delivery potentiating lipid-containing LNP versus the same LNP lacking the immune cell delivery potentiating lipid. It will be understood that the enhanced delivery of a nanoparticle to a target cell need not be determined in a subject being treated, it may be determined in a surrogate such as an animal model (e.g., a mouse or non-human primate model). For example, for determining enhanced delivery to immune cells, a mouse or NHP model can be used and delivery of an mRNA encoding a protein of interest by a immune cell delivery potentiating lipid-containing LNP can be evaluated in immune cells (e.g., from spleen, peripheral blood and/or bone marrow) (e.g., flow cytometry, fluorescence microscopy and the like) as compared to the same LNP lacking the immune cell delivery potentiating lipid.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a immune cell delivery potentiating lipid in a lipid composition (e.g., LNP) of the disclosure, an effective amount of a immune cell delivery potentiating lipid is an amount sufficient to effect a beneficial or desired result as compared to a lipid composition (e.g., LNP) lacking the immune cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering an immune cell delivery potentiating lipid-containing lipid nanoparticle such that an effective amount of lipid nanoparticles are taken up by immune cells in a subject, an effective amount of immune cell delivery potentiating lipid-containing LNP is an amount sufficient to effect a beneficial or desired result as compared to an LNP lacking the immune cell delivery potentiating lipid. Non-limiting examples of beneficial or desired results in the subject include increasing the percentage of cells transfected, increasing the level of expression of a protein encoded by a nucleic acid associated with/encapsulated by the immune cell delivery potentiating lipid-containing LNP and/or increasing a prophylactic or therapeutic effect in vivo of a nucleic acid, or its encoded protein, associated with/encapsulated by the immune cell delivery potentiating lipid-containing LNP, as compared to an LNP lacking the immune cell delivery potentiating lipid. In some embodiments, a therapeutically effective amount of immune cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of immune cells. For example, an effective amount of immune cell delivery potentiating lipid-containing LNP can be an amount that results in transfection of at least 5%, 10% or 15% of splenic T cells, at least 5%, 10%, 15%, 20% or 25% of splenic B cells and/or at least 5%, 10%, 15%, 20%, 25%, 30%, 35% or 40% of splenic dendritic cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Metastasis: As used herein, the term “metastasis” means the process by which cancer spreads from the place at which it first arose as a primary tumor to distant locations in the body. A secondary tumor that arose as a result of this process may be referred to as “a metastasis.”

Modified: As used herein “modified” or “modification” refers to a changed state or a change in composition or structure of a polynucleotide (e.g., siRNA). Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Pre-Initiation Complex (PIC): As used herein, the term “pre-initiation complex” (alternatively “43S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Residence time: As used herein, the term “residence time” refers to the time of occupancy of a pre-initiation complex (PIC) or a ribosome at a discrete position or location along an mRNA molecule.

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian immune cell) compared to an off-target cell (e.g., non-immune cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model). For example, for determining specific delivery to immune cells, a mouse or NHP model (e.g., as described in the Examples) can be used and delivery of an mRNA encoding a protein of interest can be evaluated in immune cells (e.g., from spleen, peripheral blood and/or bone marrow) as compared to non-immune cells by standard methods (e.g., flow cytometry, fluorescence microscopy and the like).

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeted cells: As used herein, “targeted cells” refers to any one or more cells of interest. The cells may be found in vitro, in vivo, in situ, or in the tissue or organ of an organism. The organism may be an animal, preferably a mammal, more preferably a human and most preferably a patient. Target immune cells include, for example, CD3+ T cells, CD19+ B cells and CD11c+ dendritic cells, as well as monocytes, tissue macrophages, and bone marrow cells (including immune cells within bone marrow, hematopoietic stem cells, immune cell precursors and fibroblasts).

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Other Embodiments of the Disclosure

The disclosure relates to the following embodiments. Throughout this section, the term embodiment is abbreviated as ‘E’ followed by an ordinal. For example, E1 is equivalent to Embodiment 1.

E1. A lipid nanoparticle comprising:

(i) an ionizable lipid;

(ii) an effective amount of a phytosterol;

(iii) optionally, a non-cationic helper lipid;

(iv) optionally, a PEG-lipid;

(v) optionally a structural lipid; and

(vi) an RNA interference agent,

wherein the effective amount of the phytosterol enhances delivery of the RNA interference agent to an immune cell relative to a lipid nanoparticle lacking the phytosterol.
E2. The lipid nanoparticle of E1, wherein an intracellular concentration of the RNA interference agent in the immune cell is enhanced.
E3. The lipid nanoparticle of E1, wherein uptake of the RNA interference agent by the immune cell is enhanced.
E4. The lipid nanoparticle of E1, wherein an activity of the RNA interference agent in the immune cell is enhanced.
E5. The lipid nanoparticle of E1, wherein expression of the RNA interference agent in the immune cell is enhanced.
E6. The lipid nanoparticle of any one of E1-E5, wherein the RNA interference agent modulates the activation or activity of the immune cell.
E7. The lipid nanoparticle of E6, wherein the RNA interference agent increases the activation or activity of the immune cell.
E8. The lipid nanoparticle of E6, wherein the RNA interference agent decreases the activation or activity of the immune cell.
E9. The lipid nanoparticle of E1, wherein the RNA interference agent is an siRNA.
E10. The lipid nanoparticle of E1, wherein expression of an mRNA targeted by the RNA interference agent in the immune cell is inhibited.
E11. The lipid nanoparticle of any one of E9-E10, wherein the mRNA targeted by the RNA interference agent modulates the activation or activity of the immune cell.
E12. The lipid nanoparticle of E11, wherein the mRNA increases the activation or activity of the immune cell.
E13. The lipid nanoparticle of E11, wherein the mRNA decreases the activation or activity of the immune cell.
E14. The lipid nanoparticle of any one of E1-E13, wherein the immune cell is selected from the group consisting of a T cell, a B cell, an NK cell, a dendritic cell, a myeloid cell and a macrophage.
E15. The lipid nanoparticle of E14, wherein the immune cell is a T cell.
E16. The lipid nanoparticle of E14, wherein the immune cell is a B cell.
E17. The lipid nanoparticle of any one of E1-E16, wherein delivery is enhanced in vivo.
E18. The lipid nanoparticle of any one of E1-E17, wherein the phytosterol has a purity of greater than 70%, greater than 80% or greater than 90%.
E19. The lipid nanoparticle of any one of E1-E17, wherein the phytosterol has a purity of greater than 95%.
E20. The lipid nanoparticle of any one of E1-E17, wherein the phytosterol has a purity of 97%, 98%, or 99%.
E21. The lipid nanoparticle of any one of E1-E20, wherein the phytosterol is a sitosterol, a stigmasterol or a combination thereof.
E22. The lipid nanoparticle of E21, wherein the phytosterol comprises a sitosterol or a salt or an ester thereof.
E23. The lipid nanoparticle of E21, wherein the phytosterol comprises a stigmasterol or a salt or an ester thereof.
E24. The lipid nanoparticle of any one of E1-E20, wherein the phytosterol is beta-sitosterol

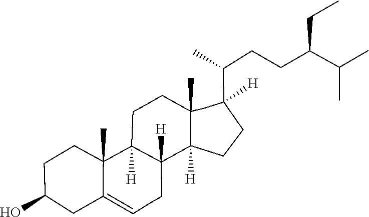

or a salt or an ester thereof.
E25. The lipid nanoparticle of E24, wherein the beta-sitosterol has a purity of greater than 70% or greater than 80%.
E26. The lipid nanoparticle of E24, wherein the beta-sitosterol has a purity of greater than 90%.
E27. The lipid nanoparticle of E24, wherein the beta-sitosterol has a purity of greater than 95%.
E28. The lipid nanoparticle of E24, wherein the beta-sitosterol has a purity of 97%, 98%, or 99%.
E29. The lipid nanoparticle of any one of E1-E28, which does not comprise a structural lipid.
E30. The lipid nanoparticle of any one of E1-E28, which comprises a structural lipid or a salt thereof.
E31. The lipid nanoparticle of E30, wherein said structural lipid is cholesterol or a salt thereof.
E32. The lipid nanoparticle of E31, wherein the mol % cholesterol is between about 1% and 50% of the mol % of phytosterol present in the lipid nanoparticle.
E33. The lipid nanoparticle of E31, wherein the mol % cholesterol is between about 10% and 40% of the mol % of phytosterol present in the lipid nanoparticle.
E34. The lipid nanoparticle of E31, wherein the mol % cholesterol is between about 20% and 30% of the mol % of phytosterol present in the lipid nanoparticle.
E35. The lipid nanoparticle of E31, wherein the mol % cholesterol is about 30% of the mol % of phytosterol present in said lipid nanoparticle.
E36. The lipid nanoparticle of any one of the preceding embodiments, wherein the ionizable lipid comprises a compound of any of Formulae (I), (IA), (II), (IIa), (IIb), (IIc), (IId), (IIe), (III), and (IIIa1-8) and/or any of Compounds X, Y, Z, Q or M.
E37. The lipid nanoparticle of any one of the preceding embodiments, wherein the ionizable lipid is at least one lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).
E38. The lipid nanoparticle of any one of the preceding embodiments, wherein the ionizable
lipid is

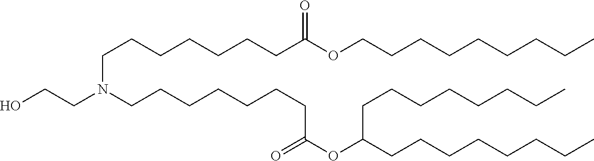 [SMILES: CCCCCCCCCOC(=O)CCCCCCCN(CCO)CCCCCCCC(=O)OC(CCCCCCCC)CCCCCCCC]

or a salt thereof.
E39. The lipid nanoparticle of any one of the preceding embodiments, wherein the ionizable lipid is

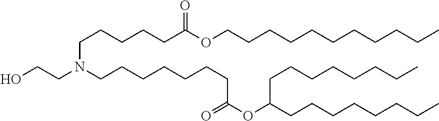 [SMILES: CCCCCCCCCCCOC(=O)CCCCCN(CCO)CCCCCCCC(=O)OC(CCCCCCCC)CCCCCCCC]

or a salt thereof.
E40. The lipid nanoparticle of any one of the preceding embodiments, which comprises a non-cationic helper lipid.
E41. The lipid nanoparticle of E40, wherein the non-cationic helper lipid is a phospholipid.
E42. The lipid nanoparticle of E41, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.
E43. The lipid nanoparticle of E42, wherein the phospholipid is DSPC.
E44. The lipid nanoparticle of E40, wherein the non-cationic helper lipid is oleic acid.
E45. The lipid nanoparticle of any one of the preceding embodiments, which comprises a PEG-lipid.
E46. The lipid nanoparticle of E45, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E47. The lipid nanoparticle of E45, wherein the PEG lipid is selected from the group consisting of PEG-c-DOMG, PEG-DMG, PEG-DLPE, PEG-DMPE, PEG-DPPC and PEG-DSPE lipid.
E48. The lipid nanoparticle of E47, wherein the PEG-lipid is PEG-DMG.
E49. The lipid nanoparticle of any one of the preceding embodiments, comprising about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.
E50. The lipid nanoparticle of any one of the preceding embodiments, comprising about 35 mol % to about 55 mol % ionizable lipid, about 5 mol % to about 25 mol % phospholipid, about 30 mol % to about 40 mol % sterol, and about 0 mol % to about 10 mol % PEG lipid.
E51. The lipid nanoparticle of any one of the preceding embodiments, comprising about 50 mol % ionizable lipid, about 10 mol % phospholipid, about 38.5 mol % sterol, and about 1.5 mol % PEG lipid.
E52. The lipid nanoparticle of any one of E1-E51, wherein the RNA interference agent is an siRNA that targets an mRNA of interest.
E53. The lipid nanoparticle of E52, wherein the siRNA targets Foxp3 mRNA.
E54. The lipid nanoparticle of E52, wherein the siRNA targets RORc mRNA.
E55. The lipid nanoparticle of E52, wherein the siRNA targets IL-17a mRNA.
E56. The lipid nanoparticle of E53, wherein the immune cell is a Treg cell.
E57. The lipid nanoparticle of E54 or E55, wherein the immune cell is a Th17 cell.
E58. The lipid nanoparticle of E52, wherein the siRNA targets an mRNA encoding a cytokine.
E59. The lipid nanoparticle of E52, wherein the siRNA targets an mRNA encoding a chemokine.
E60. The lipid nanoparticle of E52, wherein the siRNA targets an mRNA encoding a transcription factor.
E61. The lipid nanoparticle of E52, wherein the siRNA targets an mRNA encoding an intracellular adaptor protein.
E62. The lipid nanoparticle of E52, wherein the siRNA targets an mRNA encoding an intracellular signaling protein.
E63. The lipid nanoparticle of E52, wherein the siRNA targets an mRNA encoding a costimulatory molecule.
E64. The lipid nanoparticle of E52, wherein the siRNA targets an mRNA encoding an immune checkpoint molecule.
E65. The lipid nanoparticle of E52, wherein inhibition of the mRNA of interest by the siRNA decreases differentiation, activity or function of the immune cell.
E66. The lipid nanoparticle of E52, wherein inhibition of the mRNA of interest by the siRNA increases differentiation, activity or function of the immune cell.
E67. A lipid nanoparticle comprising:

(i) an ionizable lipid;

(ii) an effective amount of a phytosterol;

(iii) a non-cationic helper lipid;

(iv) a PEG-lipid; and

(v) an siRNA molecule,

wherein the effective amount of the phytosterol enhances delivery of the siRNA to an immune cell relative to a lipid nanoparticle lacking the phytosterol.
E68. The lipid nanoparticle of E67, wherein an intracellular concentration of the siRNA in the immune cell is enhanced.
E69. The lipid nanoparticle of E67, wherein uptake of the siRNA by the immune cell is enhanced.
E70. The lipid nanoparticle of E67, wherein an activity of the siRNA in the immune cell is enhanced.
E71. The lipid nanoparticle of E67, wherein expression of the siRNA in the immune cell is enhanced.
E72. The lipid nanoparticle of E67, wherein an activity of mRNA targeted by the siRNA in the immune cell is inhibited.
E73. The lipid nanoparticle of E72, wherein differentiation, activity or function of the immune cell is inhibited.
E74. The lipid nanoparticle of E72, wherein differentiation, activity or function of the immune cell is stimulated.
E75. The lipid nanoparticle of any one of E67-E74, wherein the immune cell is selected from the group consisting of a T cell, a B cell, an NK cell, a dendritic cell, a myeloid cell and a macrophage.
E76. The lipid nanoparticle of E75, wherein the immune cell is a T cell.
E77. The lipid nanoparticle of E75, wherein the immune cell is a B cell.
E78. The lipid nanoparticle of any one of E67-E77, wherein delivery of the siRNA to the immune cell is enhanced in vivo.
E79. The lipid nanoparticle of any one of E67-E78, wherein the phytosterol has a purity of greater than 70%, greater than 80% or greater than 95%.
E80. The lipid nanoparticle of any one of E67-E79, wherein the phytosterol has a purity of 97%, 98%, or 99%.
E81. The lipid nanoparticle of any one of E67-E80, wherein the phytosterol is a sitosterol, a stigmasterol or a combination thereof.
E82. The lipid nanoparticle of E81, wherein the phytosterol comprises a sitosterol or salt or ester thereof.
E83. The lipid nanoparticle of E81, wherein the phytosterol comprises a stigmasterol or salt or ester thereof.
E84. The lipid nanoparticle of any one of E67-E83, wherein the phytosterol is beta-sitosterol

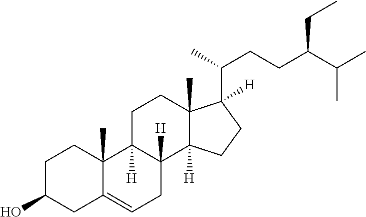

or a salt or an ester thereof.
E85. The lipid nanoparticle of E84, wherein the beta-sitosterol has a purity of greater than 70%, greater than 80%, or greater than 90%.
E86. The lipid nanoparticle of E84, wherein the beta-sitosterol has a purity of greater than 95%.
E87. The lipid nanoparticle of E84, wherein the beta-sitosterol has a purity of 97%, 98%, or 99%.
E88. The lipid nanoparticle of any one of E67-E87, which does not comprise a structural lipid.
E89. The lipid nanoparticle of any one of E67-E87, which further comprises a structural lipid or a salt thereof.
E90. The lipid nanoparticle of E89, wherein said structural lipid is cholesterol or a salt thereof.
E91. The lipid nanoparticle of E90, wherein the mol % cholesterol is between about 1% and 50% of the mol % of phytosterol present in the lipid nanoparticle.
E92. The lipid nanoparticle of E90, wherein the mol % cholesterol is between about 10% and 40% of the mol % of phytosterol present in the lipid nanoparticle.
E93. The lipid nanoparticle of E90, wherein the mol % cholesterol is between about 20% and 30% of the mol % of phytosterol present in the lipid nanoparticle.
E94. The lipid nanoparticle of E90, wherein the mol % cholesterol is about 30% of the mol % of phytosterol present in said lipid nanoparticle.
E95. The lipid nanoparticle of any one of E67-E94, wherein the ionizable lipid comprises a compound of any of Formulae (I), (IA), (II), (IIa), (IIb), (IIc), (IId), (IIe), (III), and (IIIa1-8) and/or any of Compounds X, Y, Z, Q or M.
E96. The lipid nanoparticle of any one of E67-E95, wherein the ionizable lipid is at least one lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).
E97. The lipid nanoparticle of any one of E67-E96, wherein the ionizable lipid is

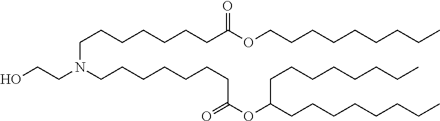 [SMILES: CCCCCCCCCOC(=O)CCCCCCCN(CCO)CCCCCCCC(=O)OC(CCCCCCCC)CCCCCCCC]

o or a salt thereof.
E98. The lipid nanoparticle of any one of E67-E96, wherein the ionizable lipid is

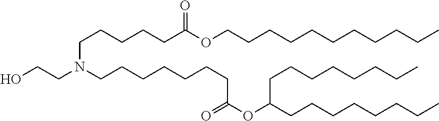 [SMILES: CCCCCCCCCCCOC(=O)CCCCCN(CCO)CCCCCCCC(=O)OC(CCCCCCCC)CCCCCCCC]

or a salt thereof.
E99. The lipid nanoparticle of any one of E67-E98, wherein the non-cationic helper lipid is a phospholipid.
E100. The lipid nanoparticle of E99, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.
E101. The lipid nanoparticle of E100, wherein the phospholipid is DSPC.
E102. The lipid nanoparticle of any one of E67-E99, wherein the non-cationic helper lipid is oleic acid.
E103. The lipid nanoparticle of any one of E67-E102, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E104. The lipid nanoparticle of E103, wherein the PEG lipid is selected from the group consisting of PEG-c-DOMG, PEG-DMG, PEG-DLPE, PEG-DMPE, PEG-DPPC and PEG-DSPE lipid.
E105. The lipid nanoparticle of E104, wherein the PEG lipid is PEG-DMG.
E106. The lipid nanoparticle of any one of E67-E105, comprising about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.
E107. The lipid nanoparticle of any one of E67-E106, comprising about 35 mol % to about 55 mol % ionizable lipid, about 5 mol % to about 25 mol % phospholipid, about 30 mol % to about 40 mol % sterol, and about 0 mol % to about 10 mol % PEG lipid.
E108. The lipid nanoparticle of any one of E67-E107, comprising about 50 mol % ionizable lipid, about 10 mol % phospholipid, about 38.5 mol % sterol, and about 1.5 mol % PEG lipid.
E109. The lipid nanoparticle of E67-108, wherein the siRNA targets Foxp3 mRNA.
E110. The lipid nanoparticle of E67-108, wherein the siRNA targets RORc mRNA.
E111. The lipid nanoparticle of E67-108, wherein the siRNA targets IL-17a mRNA.
E112. The lipid nanoparticle of E109, wherein the immune cell is a Treg cell.
E113. The lipid nanoparticle of E110 or E11, wherein the immune cell is a Th17 cell.
E114. The lipid nanoparticle of E67-108, wherein the siRNA targets an mRNA encoding a cytokine.
E115. The lipid nanoparticle of E67-108, wherein the siRNA targets an mRNA encoding a chemokine.
E116. The lipid nanoparticle of E67-108, wherein the siRNA targets an mRNA encoding a transcription factor.
E117. The lipid nanoparticle of E67-108, wherein the siRNA targets an mRNA encoding an intracellular adaptor protein.
E118. The lipid nanoparticle of E67-108, wherein the siRNA targets an mRNA encoding an intracellular signaling protein.
E119. The lipid nanoparticle of E67-108, wherein the siRNA targets an mRNA encoding a costimulatory molecule.
E120. The lipid nanoparticle of E67-108, wherein the siRNA targets an mRNA encoding an immune checkpoint molecule.
E121. The lipid nanoparticle of E67-108, wherein inhibition of the mRNA of interest by the siRNA decreases differentiation, activity or function of the immune cell.
E122. The lipid nanoparticle of E67-108, wherein inhibition of the mRNA of interest by the siRNA increases differentiation, activity or function of the immune cell.
E123. A method of delivering a nucleic acid molecule to an immune cell, the method comprising contacting the immune cell with a lipid nanoparticle (LNP) comprising:

(i) an ionizable lipid;

(ii) a phytosterol;

(iii) optionally, a non-cationic helper lipid;

(iv) optionally, a PEG-lipid;

(v) optionally, a structural lipid; and

(vi) an RNA interference agent,

such that the nucleic acid molecule is delivered to the immune cell.
E124. The method of E123, wherein the RNA interference agent is delivered to the immune cell in vivo.
E125. The method of E123, wherein an intracellular concentration of the RNA interference agent in the immune cell is enhanced.
E126. The method of E123, wherein uptake of the RNA interference agent by the immune cell is enhanced.
E127. The method of E123, wherein an activity of the RNA interference agent in the immune cell is enhanced or expression of the RNA interference agent in the immune cell is enhanced.
E128. The method of any one of E123-E127, wherein the RNA interference agent modulates the activation or activity of the immune cell.
E129. The method of E128, wherein the RNA interference agent increases the activation or activity of the immune cell.
E130. The method of E128, wherein the RNA interference agent decreases the activation or activity of the immune cell.
E131. The method of E123, wherein an activity of mRNA targeted by the RNA interference agent in the immune cell is inhibited.
E132. The method of E131, wherein the mRNA targeted by the RNA interference agent modulates the activation or activity of the immune cell.
E133. The method of E132, wherein inhibition of the mRNA targeted by the RNA interference agent increases the activation or activity of the immune cell.
E134. The method of E132, wherein the inhibition of the mRNA targeted by the RNA interference agent decreases the activation or activity of the immune cell.
E135. The method of any one of E123-E134, wherein the immune cell is a T cell.
E136. The method of any one of E123-E134, wherein the immune cell is a B cell.
E137. The method of any one of E123-E134, wherein the immune cell is selected from the group consisting of NK cells, dendritic cells, myeloid cells and macrophages.
E138. The method of any one of E123-E137, which further comprises administering, concurrently or consecutively, a second LNP encapsulating the same or different nucleic acid molecule, wherein the second LNP lacks a phytosterol.
E139. The method of any one of E123-E137, which further comprises administering, concurrently or consecutively, a second LNP encapsulating a different nucleic acid molecule, wherein the second LNP comprises a phytosterol.
E140. A method of modulating T cell activation or activity, the method comprising contacting a T cell with a lipid nanoparticle (LNP) comprising:

(i) an ionizable lipid;

(ii) a phytosterol;

(iii) optionally, a non-cationic helper lipid;

(iv) optionally, a PEG-lipid;

(v) optionally, a structural lipid; and

(vi) an RNA interference agent,

such that T cell activation or activity is modulated.
E141. The method of E140, wherein T cell activation or activity is enhanced.
E142. The method of E140, wherein T cell activation or activity is reduced.
E143. The method of any one of E140-E142, which further comprises administering, concurrently or consecutively, a second LNP encapsulating the same or different nucleic acid molecule, wherein the second LNP lacks a phytosterol.
E144. The method of any one of E140-E142, which further comprises administering, concurrently or consecutively, a second LNP encapsulating a different nucleic acid molecule, wherein the second LNP comprises a phytosterol.
E145. A method of modulating an immune response to a protein, the method comprising contacting immune cells with a lipid nanoparticle (LNP) comprising:

(i) an ionizable lipid;

(ii) a phytosterol;

(iii) optionally, a non-cationic helper lipid;

(iv) optionally, a PEG-lipid;

(v) optionally, a structural lipid; and

(vi) an RNA interference agent,

such that the immune response to the protein is increased.
E146. The method of E145, wherein the protein is an antigen.
E147. The method of E146, wherein the protein is a cancer antigen or infectious disease antigen.
E148. The method of E146, wherein the protein is an autoimmune or inflammatory antigen.
E149. The method of E145, wherein the immune cells are T cells.
E150. The method of E145, wherein the immune cells are B cells.
E151. The method of any one of E145-150, wherein the RNA interference agent targets mRNA encoding a protein different than the protein to which an immune response is enhanced.
E152. The method of any one of E145-E151, which further comprises administering, concurrently or consecutively, a second LNP encapsulating the same or different nucleic acid molecule, wherein the second LNP lacks a phytosterol.
E153. The method of any one of E145-E151, which further comprises administering, concurrently or consecutively, a second LNP encapsulating a different nucleic acid molecule, wherein the second LNP comprises a phytosterol.
E154. The method of any one of E123-E153, wherein the immune cell or T cell is contacted with the lipid nanoparticle in vitro.
E155. The method of any one of E123-E153, wherein the immune cell or T cell is contacted with the lipid nanoparticle in vivo by administering the lipid nanoparticle to a subject.
E187. The method of E155, wherein the lipid nanoparticle is administered intravenously.
E188. The method of E155, wherein the lipid nanoparticle is administered intramuscularly.
E189. The method of E155, wherein the lipid nanoparticle is administered by a route selected from the group consisting of subcutaneously, intranodally and intratumorally.
E190. The method of any one of E123-153, wherein the phytosterol comprises a stigmasterol or salt or ester thereof.
E191. The method of any one of E123-E153, wherein the phytosterol is beta-sitosterol

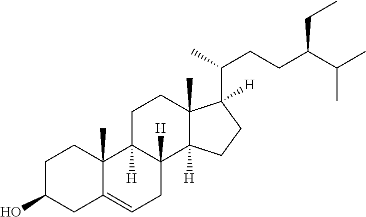

or a salt or an ester thereof.
E192. The method of E191, wherein the beta-sitosterol has a purity of greater than 70% or greater than 80% or greater than 90%.
E193. The method of E191, wherein the beta-sitosterol has a purity of greater than 95%.
E194. The method of E191, wherein the beta-sitosterol has a purity of 97%, 98%, or 99%.
E195. The method of any one of E190-E194, which does not comprise a structural lipid.
E196. The method of any one of E190-E194, wherein the lipid nanoparticle comprises a structural lipid or a salt thereof.
E197. The method of E196, wherein said structural lipid is cholesterol or a salt thereof.
E198. The method of E197, wherein the mol % cholesterol is between about 1% and 50% of the mol % of phytosterol present in the lipid nanoparticle.
E199. The method of E197, wherein the mol % cholesterol is between about 10% and 40% of the mol % of phytosterol present in the lipid nanoparticle.
E200. The method of E197, wherein the mol % cholesterol is between about 20% and 30% of the mol % of phytosterol present in the lipid nanoparticle.
E201. The method of E197, wherein the mol % cholesterol is about 30% of the mol % of phytosterol present in said lipid nanoparticle.
E202. The method of any one of E123-E201, wherein the ionizable lipid comprises a compound of any of Formulae (I), (IA), (II), (IIa), (IIb), (IIc), (IId), (IIe), (III), and (IIIa1-8) and/or any of Compounds X, Y, Z, Q or M.
E203. The method of any one of E123-E201, wherein the ionizable lipid is at least one lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).
E204. The method of any one of E123-E201, wherein the ionizable lipid is

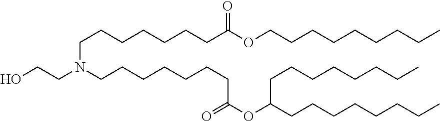 [SMILES: CCCCCCCCCOC(=O)CCCCCCCN(CCO)CCCCCCCC(=O)OC(CCCCCCCC)CCCCCCCC]

or a salt thereof.
E205. The method of any one of E123-E201, wherein the ionizable lipid is

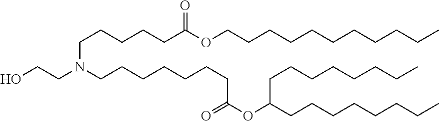 [SMILES: CCCCCCCCCCCOC(=O)CCCCCN(CCO)CCCCCCCC(=O)OC(CCCCCCCC)CCCCCCCC]

or a salt thereof.
E206. The method of any one of E123-E201, wherein the lipid nanoparticle comprises a non-cationic helper lipid.
E207. The method of E206, wherein the non-cationic helper lipid is a phospholipid.
E208. The method of E207, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.
E209. The method of E208, wherein the phospholipid is DSPC.
E210. The method of E206, wherein the non-cationic helper lipid is oleic acid.
E211. The method of any one of E123-E210, wherein the lipid nanoparticle comprises a PEG-lipid.
E212. The method of claim E211, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E213. The method of E211, wherein the PEG lipid is selected from the group consisting of PEG-c-DOMG, PEG-DMG, PEG-DLPE, PEG-DMPE, PEG-DPPC and PEG-DSPE lipid.
E214. The method of E213, wherein the PEG lipid is PEG-DMG.
E215. The method of any one of E123-E214, wherein the lipid nanoparticle comprises about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.
E216. The method of any one of E123-E214, wherein the lipid nanoparticle comprises about 35 mol % to about 55 mol % ionizable lipid, about 5 mol % to about 25 mol % phospholipid, about 30 mol % to about 40 mol % sterol, and about 0 mol % to about 10 mol % PEG lipid.
E217. The method of any one of E123-E214, wherein the lipid nanoparticle comprises about 50 mol % ionizable lipid, about 10 mol % phospholipid, about 38.5 mol % sterol, and about 1.5 mol % PEG lipid.
E218. The method of any one of E123-217 and 185-230, wherein the RNA interference agent is an siRNA.
E219. A method of modulating an immune response in a subject, the method comprising administering to the subject a lipid nanoparticle (LNP) comprising:

(i) an ionizable lipid;

(ii) a phytosterol;

(iii) optionally, a non-cationic helper lipid;

(iv) optionally, a PEG-lipid;

(v) optionally, a structural lipid; and

(vi) an RNA interference agent,

wherein the LNP comprises a phytosterol such that an immune response is modulated in the subject, as compared to the immune response induced by an LNP encapsulating the RNA interference agent but lacking the phytosterol.
E220. The method of E219, wherein the RNA interference agent is an siRNA.
E221. The method of E220, wherein the siRNA targets an mRNA encoding Foxp3.
E222. The method of E220, wherein the siRNA targets an mRNA encoding RORc.
E223. The method of E220, wherein the siRNA targets an mRNA encoding IL-17a.
E224. The method of any one of E219-E223, wherein the lipid nanoparticle is administered intramuscularly.
E225. The method of any one of E219-E223, wherein the lipid nanoparticle is administered intradermally.
E226. The method of any one of E219-E223, wherein the lipid nanoparticle is administered intranodally.
E227. The method of any one of E219-E226, wherein the immune response is an antigen-specific antibody response.
E228. The method of E219-E226, wherein the immune response is an antigen-specific T cell response.
E229. The method of any one of E219-E228, which further comprises administering, concurrently or consecutively, a second LNP encapsulating the same or different RNA interference agent, wherein the second LNP lacks a phytosterol.
E230. The method of any one of E219-E228, which further comprises administering, concurrently or consecutively, a second LNP encapsulating a different nucleic acid molecule, wherein the second LNP comprises a phytosterol.
E231. The method of any one of E219-E230, wherein the phytosterol has a purity of greater than 70%.
E232. The method of any one of E219-E230, wherein the phytosterol has a purity of greater than 80%.
E233. The method of any one of E219-E230, wherein the phytosterol has a purity of greater than 90%.
E234. The method of any one of E219-E230, wherein the phytosterol has a purity of greater than 95%.
E235. The method of any one of E219-E230, wherein the phytosterol has a purity of 97%, 98%, or 99%.
E236. The method of any one of E219-E235, wherein the phytosterol is a sitosterol, a stigmasterol or a combination thereof.
E237. The method of E236, wherein the phytosterol comprises a sitosterol or salt or ester thereof.
E238. The method of E236, wherein the phytosterol comprises a stigmasterol or salt or ester thereof.
E239. The method of any one of E219-E235, wherein the phytosterol is beta-sitosterol

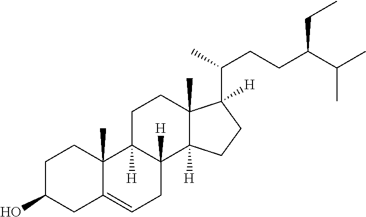

or a salt or an ester thereof.
E240. The method of E239, wherein the beta-sitosterol has a purity of greater than 70% or greater than 80%.
E241. The method of E239, wherein the beta-sitosterol has a purity of greater than 90%.
E242. The method of E239, wherein the beta-sitosterol has a purity of greater than 95%.
E243. The method of E239, wherein the beta-sitosterol has a purity of 97%, 98%, or 99%.
E244. The method of any one of E219-E243, which does not comprise a structural lipid.
E245. The method of any one of E219-E243, wherein the lipid nanoparticle comprises a structural lipid or salt thereof.
E246. The method of E245, wherein said structural lipid is cholesterol or a salt thereof.
E247. The method of E246, wherein the mol % cholesterol is between about 1% and 50% of the mol % of phytosterol present in the lipid nanoparticle.
E248. The method of E246, wherein the mol % cholesterol is between about 10% and 40% of the mol % of phytosterol present in the lipid nanoparticle.
E249. The method of E246, wherein the mol % cholesterol is between about 20% and 30% of the mol % of phytosterol present in the lipid nanoparticle.
E250. The method of E246, wherein the mol % cholesterol is about 30% of the mol % of phytosterol present in said lipid nanoparticle.
E251. The method of any one of E219-E250, wherein the ionizable lipid comprises a compound of any of Formulae (I), (IA), (II), (IIa), (IIb), (IIc), (IId), (IIe), (III), and (IIIa1-8) and/or any of Compounds X, Y, Z, Q or M.
E252. The method of any one of E219-E250, wherein the ionizable lipid is at least one lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).
E253. The method of any one of E219-E250, wherein the ionizable lipid is

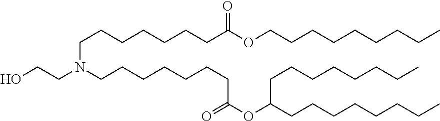 [SMILES: CCCCCCCCCOC(=O)CCCCCCCN(CCO)CCCCCCCC(=O)OC(CCCCCCCC)CCCCCCCC]

or a salt thereof.
E254. The method of any one of E219-E250, wherein the ionizable lipid is

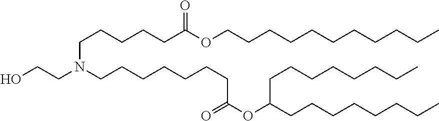 [SMILES: CCCCCCCCCCCOC(=O)CCCCCN(CCO)CCCCCCCC(=O)OC(CCCCCCCC)CCCCCCCC]

or a salt thereof.
E255. The method of any one of E219-E254, wherein the lipid nanoparticle comprises a non-cationic helper lipid.
E256. The method of E255, wherein the non-cationic helper lipid is a phospholipid.
E257. The method of E256, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.
E258. The method of E257, wherein the phospholipid is DSPC.
E259. The method of E255, wherein the non-cationic helper lipid is oleic acid.
E260. The method of any one of E219-E259, wherein the lipid nanoparticle comprises a PEG-lipid.
E261. The method of E260, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E262. The method of 260, wherein the PEG lipid is selected from the group consisting of PEG-c-DOMG, PEG-DMG, PEG-DLPE, PEG-DMPE, PEG-DPPC and PEG-DSPE lipid.
E263. The method of E262, wherein the PEG lipid is PEG-DMG.
E264. The method of any one of E219-E263, wherein the lipid nanoparticle comprises about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.
E264. The method of any one of E219-E263, wherein the lipid nanoparticle comprises about 35 mol % to about 55 mol % ionizable lipid, about 5 mol % to about 25 mol % phospholipid, about 30 mol % to about 40 mol % sterol, and about 0 mol % to about 10 mol % PEG lipid.
E265. The method of any one of E219-E263, wherein the lipid nanoparticle comprises about 50 mol % ionizable lipid, about 10 mol % phospholipid, about 38.5 mol % sterol, and about 1.5 mol % PEG lipid.
E266. A method of modulating B cell activation or activity, the method comprising contacting a B cell with a lipid nanoparticle (LNP) comprising:

(i) an ionizable lipid;

(ii) a phytosterol;

(iii) optionally, a non-cationic helper lipid;

(iv) optionally, a PEG-lipid;

(v) optionally, a structural lipid; and

(vi) an RNA interference agent,

such that B cell activation or activity is modulated.
E267. The method of E266, wherein B cell activation or activity is enhanced.
E268. The method of E266, wherein B cell activation or activity is reduced.
E269. The method of any one of E266-E270, which further comprises administering, concurrently or consecutively, a second LNP encapsulating the same or different RNA interference agent, wherein the second LNP lacks a phytosterol.
E270. The method of any one of E266-E270, which further comprises administering, concurrently or consecutively, a second LNP encapsulating a different nucleic acid molecule, wherein the second LNP comprises a phytosterol.
E271. The method of any one of E266-E272, wherein the B cell is contacted with the lipid nanoparticle in vitro.
E272. The method of any one of E266-E272, wherein the B cell is contacted with the lipid nanoparticle in vivo by administering the lipid nanoparticle to a subject.
E273. The method of E272, wherein the lipid nanoparticle is administered intravenously.
E274. The method of E272, wherein the lipid nanoparticle is administered intramuscularly.
E275. The method of E272, wherein the lipid nanoparticle is administered by a route selected from the group consisting of subcutaneously, intranodally and intratumorally.
E276. The method of any one of E266-E275, wherein an intracellular concentration of the RNA interference agent in the B cell is enhanced.
E277. The method of any one of E266-E275, wherein an activity of the RNA interference agent in the B cell is enhanced.
E278. The method of any one of E266-E275, wherein expression of the RNA interference agent in the B cell is enhanced.
E279. The method of any one of E266-E275, wherein the RNA interference agent modulates the activation or activity of the B cell.
E280. The method of E279, wherein the RNA interference agent increases the activation or activity of the B cell.
E281. The method of E279, wherein the RNA interference agent decreases the activation or activity of the B cell.
E282. The method of any one of E266-E281, wherein the phytosterol has a purity of greater than 70%, greater than 80%, greater than 90% or greater than 95%.
E283. The method of any one of E266-E281, wherein the phytosterol has a purity of 97%, 98%, or 99%.
E284. The method of any one of E266-E283, wherein the phytosterol is a sitosterol, a stigmasterol or a combination thereof.
E285. The method of E284, wherein the phytosterol comprises a sitosterol or salt or ester thereof.
E286. The method of E284, wherein the phytosterol comprises a stigmasterol or salt or ester thereof.
E287. The method of any one of E266-E283, wherein the phytosterol is beta-sitosterol

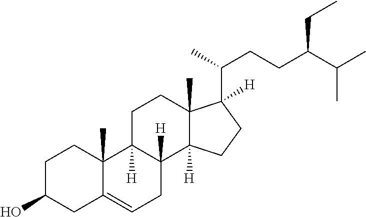

or a salt or an ester thereof.
E288. The method of E287, wherein the beta-sitosterol has a purity of greater than 70% or greater than 80% or greater than 90%.
E289. The method of E287, wherein the beta-sitosterol has a purity of greater than 95%.
E290. The method of E287, wherein the beta-sitosterol has a purity of 97%, 98%, or 99%.
E291. The method of any one of E266-E290, which does not comprise a structural lipid.
E292. The method of any one of E266-E291, wherein the lipid nanoparticle comprises a structural lipid or a salt thereof.
E293. The method of E292, wherein said structural lipid is cholesterol or a salt thereof.
E294. The method of E293, wherein the mol % cholesterol is between about 1% and 50% of the mol % of phytosterol present in the lipid nanoparticle.
E295. The method of E293, wherein the mol % cholesterol is between about 10% and 40% of the mol % of phytosterol present in the lipid nanoparticle.
E296. The method of E293, wherein the mol % cholesterol is between about 20% and 30% of the mol % of phytosterol present in the lipid nanoparticle.
E297. The method of E293, wherein the mol % cholesterol is about 30% of the mol % of phytosterol present in said lipid nanoparticle.
E298. The method of any one of E266-E297, wherein the ionizable lipid comprises a compound of any of Formulae (I), (IA), (II), (IIa), (IIb), (IIc), (IId), (IIe), (III), and (IIIa1-8) and/or any of Compounds X, Y, Z, Q or M.
E299. The method of any one of E266-E297, wherein the ionizable lipid is at least one lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).
E302. The method of any one of E267-E299, wherein the ionizable lipid is

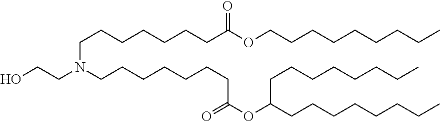 [SMILES: CCCCCCCCCOC(=O)CCCCCCCN(CCO)CCCCCCCC(=O)OC(CCCCCCCC)CCCCCCCC]

or a salt thereof.
E300. The method of any one of E266-E297, wherein the ionizable lipid is

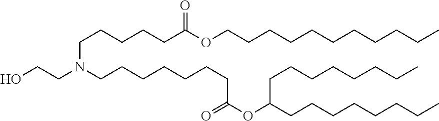 [SMILES: CCCCCCCCCCCOC(=O)CCCCCN(CCO)CCCCCCCC(=O)OC(CCCCCCCC)CCCCCCCC]

or a salt thereof.
E301. The method of any one of E266-E297, wherein the lipid nanoparticle comprises a non-cationic helper lipid.
E302. The method of E301, wherein the non-cationic helper lipid is a phospholipid.
E303. The method of E302, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.
E304. The method of E303, wherein the phospholipid is DSPC.
E305. The method of E301, wherein the non-cationic helper lipid is oleic acid.
E306. The method of any one of E266-E305, wherein the lipid nanoparticle comprises a PEG-lipid.
E307. The method of E306, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E308. The method of E307, wherein the PEG lipid is selected from the group consisting of PEG-c-DOMG, PEG-DMG, PEG-DLPE, PEG-DMPE, PEG-DPPC and PEG-DSPE lipid.
E309. The method of E308, wherein the PEG lipid is PEG-DMG.
E310. The method of any one of E266-E309, wherein the lipid nanoparticle comprises about 30 mol % to about 60 mol % ionizable lipid, about 0 mol % to about 30 mol % phospholipid, about 18.5 mol % to about 48.5 mol % sterol, and about 0 mol % to about 10 mol % PEG lipid.
E311. The method of any one of E266-E310, wherein the lipid nanoparticle comprises about 35 mol % to about 55 mol % ionizable lipid, about 5 mol % to about 25 mol % phospholipid, about 30 mol % to about 40 mol % sterol, and about 0 mol % to about 10 mol % PEG lipid.
E312. The method of any one of E266-E311, wherein the lipid nanoparticle comprises about 50 mol % ionizable lipid, about 10 mol % phospholipid, about 38.5 mol % sterol, and about 1.5 mol % PEG lipid.
E313. The method of any one of E266-E312, wherein the RNA interference agent is an siRNA.
E314. The method of E313, wherein the siRNA targets an mRNA encoding a cytokine.
E315. The method of E313, wherein the siRNA targets an mRNA encoding a chemokine.
E316. The method of E313, wherein the siRNA targets an mRNA encoding a transcription factor.
E317. The method of E313, wherein the siRNA targets an mRNA encoding an intracellular adaptor protein.
E318. The method of E313, wherein the siRNA targets an mRNA encoding an intracellular signaling protein.
E319. A lipid nanoparticle (LNP) for use in a method of immune therapy with enhanced delivery to an immune cell,

wherein the LNP comprises:
(i) a sterol or other structural lipid;
(ii) an ionizable lipid; and
(iii) an RNA interference agent for delivery to an immune cell;

wherein one or more of (i) the sterol or other structural lipid and/or (ii) the ionizable lipid comprises an immune cell delivery potentiating lipid in an amount effective to enhance delivery of the LNP to an immune cell,

wherein the enhanced delivery is a characteristic of said LNP relative to a control LNP lacking the immune cell delivery potentiating lipid.

E320. The LNP for use of E319, wherein the sterol or other structural lipid is a phytosterol or cholesterol.
E321. The LNP for use of E319 or E320, wherein the immune cell delivery potentiating lipid binds to C1q and/or promotes the binding of the LNP comprising said lipid to C1q compared to a control LNP lacking the immune cell delivery potentiating lipid and/or increases uptake of C1q-bound LNP into an immune cell compared to a control LNP lacking the immune cell delivery potentiating lipid.
E322. The LNP for use of any one of E319-E321, wherein the RNA interference agent is a small interfering RNA (siRNA).
E323. The LNP for use of E322, wherein in the siRNA targets an mRNA encoding a transcription factor in the immune cell.
E324. The LNP for use of E322, wherein in the siRNA targets an mRNA encoding a cytokine in the immune cell.
E325. The LNP for use of E322, wherein in the siRNA targets an mRNA encoding a receptor in the immune cell.
E326. The LNP for use of E322, wherein in the siRNA targets an mRNA encoding a signaling molecule in the immune cell.
E327. The LNP for use of E319-E326, wherein the immune cell is a lymphocyte.
E328. The LNP for use of E319-E327, wherein the immune cell is a T cell.
E329. The LNP for use of E319-E327, wherein the immune cell is a B cell.
E330. The LNP for use of E319-326, wherein the immune cell is an NK cell, a dendritic cell, a myeloid cell or a macrophage.
E331. The LNP for use of E319-E330, wherein the lipid nanoparticle further comprises:

(vi) a non-cationic helper lipid or phospholipid, and/or

(v) a PEG-lipid.

E332. The LNP for use of E319-E331, wherein the sterol or other structural lipid comprises a phytosterol selected from the group consisting of β-sitosterol, stigmasterol, β-sitostanol, campesterol, brassicasterol, and combinations thereof. In one embodiment, the phytosterol is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.
E333. The LNP for use of E319-E332, wherein the method results in modulation of activation or activity of an immune cell.
E334. The LNP for use of E319-E333, wherein the method results in modulation of activation or activity of a T cell.
E335. The LNP for use of E328, wherein the siRNA targets FoxP3 mRNA and the T cell is a regulatory T cell (Treg).
E336. The LNP for use of E328, wherein the siRNA targets RORc mRNA and the T cell is a Th17 cell.
E337. The LNP for use of E328, wherein the siRNA targets IL-17a mRNA and the T cell is a Th17 cell.
E338. A pharmaceutical composition comprising the lipid nanoparticle of E319-E337 and a pharmaceutically acceptable carrier.
E339. Use of a lipid nanoparticle of E319-E337, and an optional pharmaceutically acceptable carrier, in the manufacture of a medicament for modulating an immune response in an individual, wherein the medicament comprises the lipid nanoparticle and an optional pharmaceutically acceptable carrier and wherein the treatment comprises administration of the medicament, and an optional pharmaceutically acceptable carrier.
E340. A kit comprising a container comprising the lipid nanoparticle of E319-E337, and an optional pharmaceutically acceptable carrier, and a package insert comprising instructions for administration of the lipid nanoparticle for modulating an immune response in an individual.
E341. An in vitro method of delivering an RNA interference agent to an immune cell, the method comprising contacting the immune cell with an LNP as defined in E319-E337, which comprises an immune cell delivery potentiating lipid.
E342. The in vitro method of E341, wherein the method results in modulation of activation or activity of the immune cell.
E342. A method of modulating an immune response in a subject, the method comprising administering to the subject the lipid nanoparticle of E319-E337, and an optional pharmaceutically acceptable carrier, such that an immune response is modulated in the subject.
E343. The method of E342, wherein an immune response is stimulated in the subject.
E344. The method of E342, wherein an immune response is inhibited in the subject.
E345. The method of E342, wherein modulation of the immune response comprises modulation of cytokine production.
E346. The method of E342, wherein modulation of the immune response comprises modulation of immune cell proliferation.
E347. The method of E342, wherein modulation of the immune response comprises modulation of at least one effector function of an immune cell.
E348. The method of E342, wherein modulation of the immune response comprises modulation of immunoglobulin production.
E349. The method of E343, wherein the subject is suffering from cancer.
E350. The method of E343, wherein the subject is suffering from an infectious disease.
E351. The method of E343, wherein the subject is receiving or has received a vaccine and the immune response to the vaccine is stimulated.
E352. The method of E344, wherein the subject has an autoimmune disease, is suspected of having an autoimmune disease or is at risk of developing an autoimmune disease.
E353. The method of E344, wherein the subject has an allergic disorder.
E354. The method of E344, wherein the subject has an inflammatory disorder.
E355. The method of E344, wherein the subject is a transplant recipient.
E356. The method of E344, wherein the subject is undergoing immunotherapy.
E357. The method of E342-E356, wherein the subject is administered at least one additional immunomodulatory agent.
E358. A method of modulating a T cell response in a subject, the method comprising administering to the subject the lipid nanoparticle of E319-E337, and an optional pharmaceutically acceptable carrier, such that a T cell response is modulated in the subject.
E359. The method of E358, wherein a T cell response is stimulated in the subject.
E360. The method of E358, wherein a T cell response is inhibited in the subject.
E361. The method of E358, wherein the RNA interference agent is an siRNA.
E362. The method of E358, wherein the siRNA targets mRNA encoding a transcription factor.
E363. The method of E362, wherein the transcription factor is a Foxp3 transcription factor.
E364. The method of E362, wherein the transcription factor is a ROR transcription factor.
E365. The method of E358, wherein the siRNA targets mRNA encoding a cytokine.
E366. The method of E365, wherein the cytokine is IL-17a.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Downregulation of Foxp3 Expression in In Vitro Differentiated Regulatory T Cells Using LNP-Encapsulated Foxp3 siRNA

In this example, Foxp3 siRNA constructs from three commercial sources were encapsulated in LNPs for targeting delivery to immune cells. The mouse Foxp3 sequences targeted by the siRNAs are shown in Genbank accession numbers NM_001199347.1, NM_001199348.1 and NM_054039.2. The tested siRNA preparations targeted various different regions of the Foxp3 gene, as shown below in Table 17:

TABLE 17
Foxp3 siRNA Constructs

| | | Targeted |
|---|---|---|
| | | Gene |
| Label | siRNA | region |
| Scrambled siRNA | siRNA 0 | n/a |
| Vendor 1 Foxp3 siRNA | siRNA 1 | Exon 12 |
| Vendor 2 Foxp3 siRNA | siRNA 2 | ORF |
| Group 1 | siRNA 3 | ORF |
| | siRNA 4 | ORF |
| | siRNA 5 | ORF |
| Vendor 2 Foxp3 siRNA | siRNA 6 | 3′UTR |
| Group 2 | siRNA 7 | ORF |
| | siRNA 8 | 3′UTR |
| | siRNA 9 | 3′UTR |

As indicated in Table 17, the Vendor 1 Foxp3 siRNA is a single siRNA construct, whereas the Vendor 2 Foxp3 siRNAs (Groups 1 and 2) are each pools of four siRNAs. Scrambled siRNA is the negative control siRNA.

The siRNA constructs were purchased from commercial vendors. The siRNA constructs were formulated into lipid nanoparticles comprising Compound X/DSPC/cholesterol/beta-sitosterol/PEG DMG at a ratio of 50:10:10:28.5:1.5. Such lipid nanoparticles (LNPs), which contain beta-sitosterol as an immune cell delivery potentiating lipid, are described further in PCT Application No. PCT/US19/15913, filed Jan. 30, 2019, the entire contents of which is expressly incorporated herein by reference.

Initial experiments were performed using in vitro differentiated mouse regulatory T cells. Naïve mouse CD4+ T cells were enriched from total mouse splenocytes using the EasySep™ mouse naïve CD4+ T cell isolation kit (StemCell Technologies). Cells were seeded at 1-3×10⁶ cells/mL cultured on plates coated with 3 g/mL anti-CD3F (eBioscience) in complete RPMI medium containing 5 ng/mL each of rhIL-2 and TGF-β (both from BioLegend) for 5 days, refreshing the medium with rhIL-2 on day 3. On day 5, cells were washed and incubated with LNP-encapsulated siRNA for 24 to 48 hours and then analyzed for Foxp3 expression by flow cytometry. siRNA doses were 1 μg/mL on 1.0×10⁶ cells/mL.

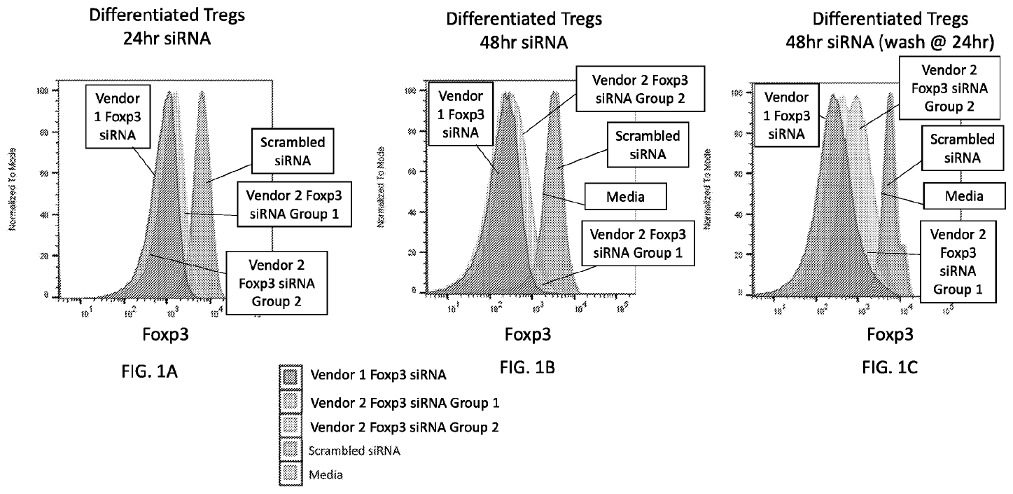

The results are shown in FIGS. 1A-1C. All of the Foxp3 siRNAs decreased Foxp3 MFI on differentiated Tregs compared to scrambled siRNA and media after 24 and 48 hours of exposure (FIGS. 1A-1B). This trend also applied to differentiated Tregs where siRNA was washed away after 24 hours and then analyzed the following day (FIG. 1C). There was a subtle but consistent increase in knock down observed with the Vendor 1 Foxp3 siRNA compared to the other Foxp3 siRNA tested. Therefore, the Vendor 1 Foxp3 siRNA construct was selected for further investigation.

### Example 2: Downregulation of Foxp3 Expression in Splenocytes and Ex Vivo Regulatory T Cells Using LNP-Encapsulated Foxp3 siRNA

Based on the results described in Example 1 showing downregulation of Foxp3 expression in in vitro differentiated mouse Treg cells by LNP-encapsulated Vendor 1 Foxp3 siRNA, the LNP formulation prepared as described in Example 1 was further tested on total mouse splenocytes and on mouse ex vivo Treg cells. Mouse splenocytes were plated in RPMI medium and immediately cultured for 24 hours with either 10 μg/mL or 1 μg/mL LNP-encapsulated siRNA (10× and 1×, respectively). Ex vivo Tregs were obtained by magnetically sorting cells from naïve C57BL/6 mice using a mouse Treg isolation kit (StemCell Technologies). Ex vivo Tregs were then incubated with 1 μg/ml LNP-encapsulated siRNA for 24 h. The control was a scrambled siRNA.

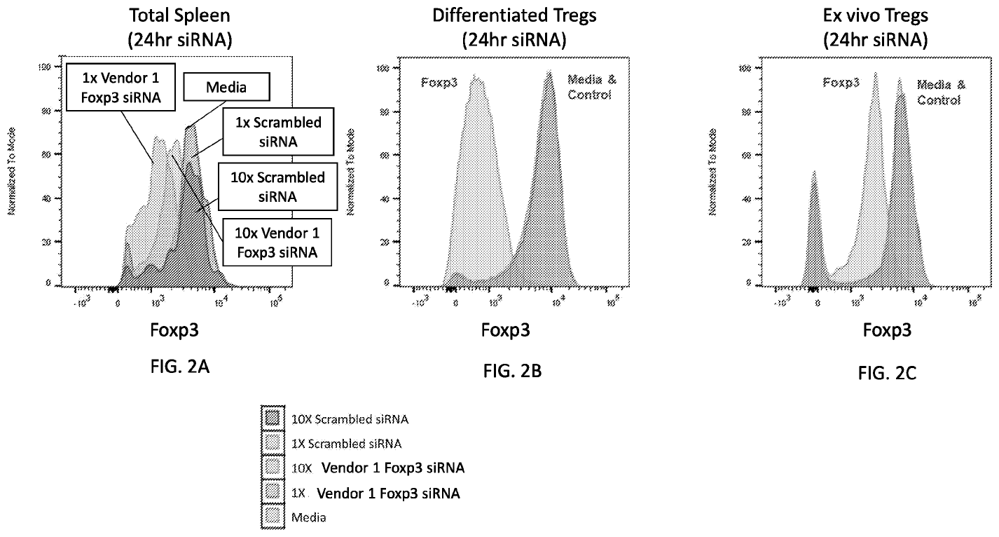

The results are shown in FIGS. 2A-2C. For the splenocytes (FIG. 2A), inhibition of Foxp3 expression was observed in a dose-dependent manner, where Foxp3 siRNA decreased Foxp3 MFI within CD4+ T cells. Inhibition of FoxP3 expression by the siRNA was also observed in in vitro differentiated Tregs (FIG. 2B), consistent with the results described in Example 1, and in the ex vivo Tregs (FIG. 2C). Thus, these data show that Foxp3 expression can be diminished in differentiated Tregs, steady state ex vivo Tregs, and within CD4+ T cells from a total splenocyte preparation by the LNP-encapsulated Foxp3 siRNA.

### Example 3: Dose Dependent Downregulation of Foxp3 Expression by LNP-Encapsulated Foxp3 siRNA

In this example, a dose titration of Foxp3 siRNA was performed on in vitro differentiated Tregs, testing serial five-fold dilutions starting at a top dose of 1,000 ng/mL. Differentiated Tregs were seeded at 1.0×10⁶ cells/mL and cultured with the following doses of LNP-encapsulated siRNA: 1.6 ng/mL, 8 ng/mL, 40 ng/mL, 200 ng/mL or 1000 ng/ml. The control was a scrambled siRNA. LNP-encapsulated Vendor 1 Foxp3 siRNA was formulated as described in Example 1.

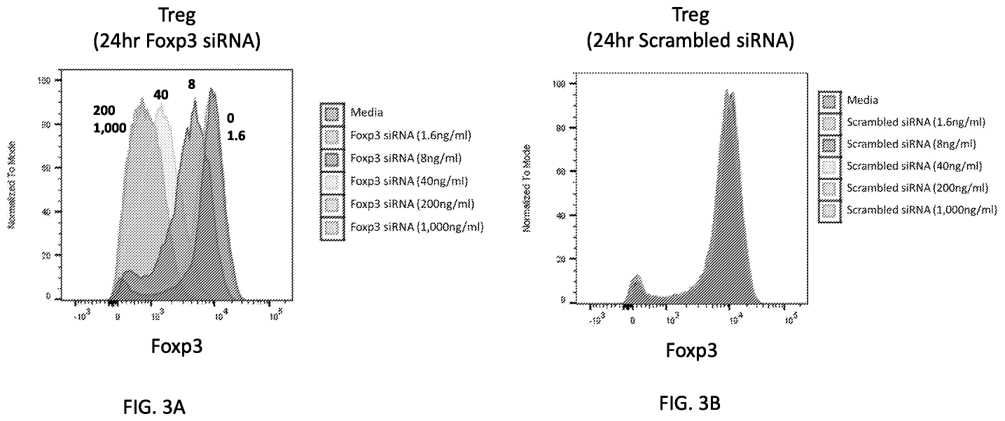

The results are shown in FIGS. 3A-3B. Foxp3 knock down was observed in a dose dependent manner during a 24 h incubation where Foxp3 siRNA had an effect of knock down as low as 5 ng/mL (FIG. 3A). No knock down was observed with scrambled siRNA for a 24 h incubation at equivalent doses (FIG. 3B).

### Example 4: Inhibition of Regulatory T Cell Differentiation by LNP-Encapsulated Foxp3 siRNA

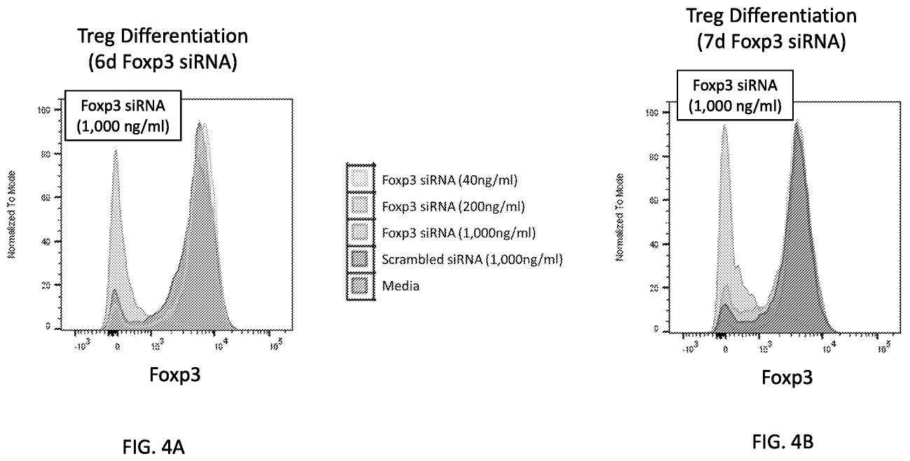

In this example, the effect of LNP-encapsulated Foxp3 siRNA on Treg differentiation in vitro was examined. Naïve CD4+ mouse T cells were isolated and cultured in Treg medium, as described in Example 1, to promote the differentiation to Tregs. LNP-encapsulated Foxp3 or scrambled siRNA was added at the beginning of culture (40 ng/ml-1,000 ng/ml) and then Foxp3 MFI was measured within the live CD4+ T cell population after 6 and 7 days of culture. LNP-encapsulated Vendor 1 Foxp3 siRNA was formulated as described in Example 1. The results are shown in FIGS. 4A (day 6) and 4B (day 7). After both 6 and 7 days of culture, 1,000 ng/ml LNP-encapsulated Foxp3 siRNA inhibited the differentiation of Tregs, while lower doses or scrambled siRNA did not affect Foxp3 MFI compared to the media alone control. These data show that the LNP-encapsulated Foxp3 siRNA can inhibit the in vitro differentiation of Tregs from naïve CD4 cells at a high dose.

### Example 5: Inhibition of Treg Suppression of Teff Cells by LNP-Encapsulated Foxp3 siRNA

Since Foxp3 siRNA was observed to knock down Foxp3 in differentiated Tregs (as described in Examples 1-3), in this example the consequence of Foxp3 knock down on Treg function was examined. Differentiated mouse Tregs were mixed with mouse T effector cells (Teff; CD4+CD25−) for six days and the proliferation of Teff was measured. Tregs from Foxp3− GFP mice (Jackson Labs) were differentiated for 5 days as described in Example 1, cultured for 24 h with the LNP-encapsulated Foxp3 siRNA (or control scrambled siRNA), and then the CD4+GFP+ cells (Treg) were purified by the Sony SH800 cell sorter. Teff were prepared by sorting CD4+CD25− cells from CD45.1 mice and then labeling with CellTrace Violet 1:1000 (ThermoFisher) for 20 min, followed by several washes. Teff were plated at 100,000 cells/well in a 96-well plate. Tregs were added at a top number of 100,000 cells/well (1:1 Treg:Teff ratio) and then diluted 2-fold down to 1:16 ratio of Treg:Teff. 100,000 cells from Rag−/− mice and 1 g/mL anti-CD3F (eBioscience) were added to each well to provide a stimulus. Proliferation of Teff was determined by CTV dilution, where % proliferation represents the % of live CD4+CD45.1+ cells that divided. LNP-encapsulated Vendor 1 Foxp3 siRNA was formulated as described in Example 1.

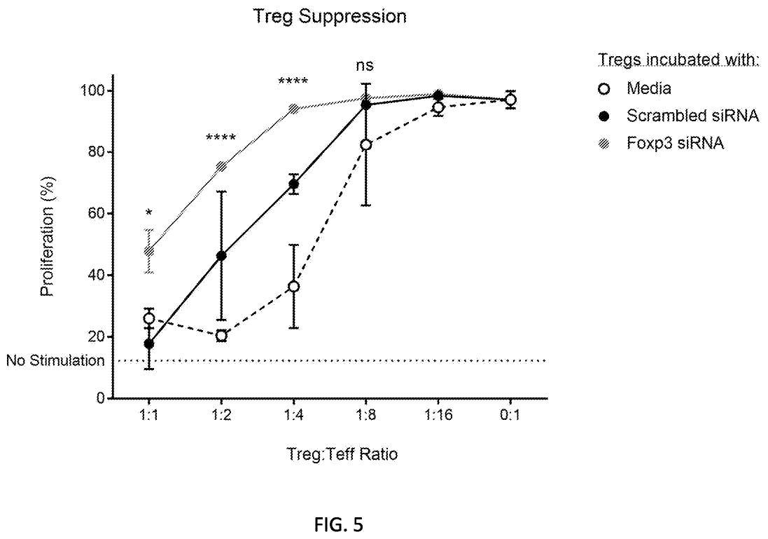

The results are shown in the graph of FIG. 5, wherein the x-axis shows the Treg:Teff ratio and the y-axis shows the percentage of proliferated Teff cells enumerated by CTV dilution. The dotted line represents the amount of Teff proliferation with no stimulation, where no anti-CD3P was added to the 0:1 Treg:Teff condition. Statistical significance was determined by 2-way ANOVA followed by a Tukey post-test, where significance compares Tregs incubated with Foxp3 siRNA to Tregs with media. These data show that Teff cells were able to proliferate significantly more when cultured with Tregs incubated with LNP-encapsulated Foxp3 siRNA, as compared to Tregs incubated with media or control siRNA, at Treg:Teff ratios of 1:1, 1:2, and 1:4. No significant inhibition of proliferation was observed at Treg:Teff ratios of 1:8 or 1:16. These data demonstrate that the LNP-encapsulated Foxp3 siRNA causes disruption of the suppressive function of Tregs in vitro.

### Example 6: Downregulation of IL-17a Expression in Th17 Cells Using LNP-Encapsulated RORc and IL-17a siRNA

In this example, naïve CD4+ T cells from mice were Th17 differentiated in vitro and tested for whether LNPs encapsulating siRNA against RORc or IL-17a could affect IL-17a expression by the Th17 cells. Commercially-available RORc and IL-17a siRNAs were used. The mouse RORc sequences targeted by the siRNAs are shown in Genbank accession numbers NR_121656.1, NM_011281.3, XM_006501162.3, XM_006501163.2 and XM_001293734.1. The mouse IL-17a sequence targeted by the siRNAs is shown in Genbank accession number NM_010552.3. The tested siRNA preparations targeted various different regions of the RORc or IL-17a genes, as shown below in Table 18:

TABLE 18
RORc and IL-17a siRNA Constructs

| | siRNA | |
|---|---|---|
| | catalogue | Gene |
| Label | number | region |
| Scrambled siRNA | siRNA 0 | n/a |
| Vendor 2 RORc siRNA | siRNA 1 | 3′UTR |
| | siRNA 2 | ORF |
| | siRNA 3 | 3′UTR |
| | siRNA 4 | 3′UTR |
| Vendor 2 IL-17a siRNA | siRNA 5 | ORF |
| | siRNA 6 | 3′UTR |
| | siRNA 7 | ORF |
| | siRNA 8 | ORF |

As indicated in Table 18, the Vendor 2 RORc siRNA and the IL-17a siRNAs are each pools of four siRNAs. Scrambled siRNA is the negative control siRNA.

The siRNA constructs were formulated into lipid nanoparticles comprising Compound X/DSPC/cholesterol/beta-sitosterol/PEG DMG at a ratio of 50:10:10:28.5:1.5. Such lipid nanoparticles (LNPs), which contain beta-sitosterol as an immune cell delivery potentiating lipid, are described further in PCT Application No. PCT/US19/15913, filed Jan. 30, 2019, the entire contents of which is expressly incorporated herein by reference.

The Th17 differentiation was performed as follows: naïve CD4+ T cells were isolated from C57BL/6 mice using the mouse naïve CD4+ T cell isolation kit (StemCell) and then cultured for 5 days using the mouse Th17 differentiation kit according to the manufacturer's instructions (R&D systems). On day 5, either scrambled, RORc, or IL-17a siRNA, encapsulated in LNPs, was added to the cultures containing 400,000 cells/mL from 0.01 to 100 μg/mL. Cells were harvested after 24 h and 48 h and then stimulated with phorbol 12-myristate 13-acetate (PMA), ionomycin, and brefeldin A for 6 h to amplify intracellular cytokine signal. Flow cytometry was performed to measure the MFI of IL-17a within the live CD4+ T cell population.

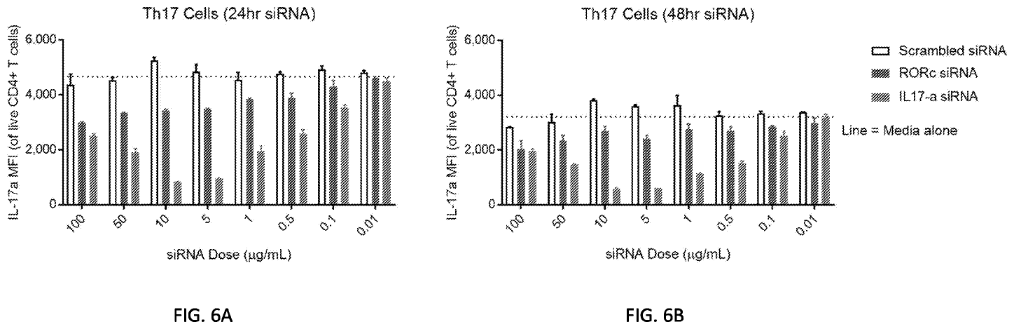

The results are shown in FIGS. 6A (24 hours) and 6B (48 hours). The dotted line represents IL-17a MFI of cells that did not receive siRNA. The results demonstrate that the LNP-encapsulated RORc and IL-17a siRNA decreased the MFI of IL-17a down to doses of about 0.1 μg/mL at 24 h and 48 h of incubation, showing that both these siRNA pools, but not the scrambled siRNA, was able to knock down IL-17a expression in the Th17 cells. These data provide evidence that LNPs encapsulating siRNAs that target proteins expressed in Th17 cells can alter IL-17a gene expression within the Th17 differentiated cells.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

All references described herein are incorporated by reference in their entireties.

What is claimed is:

1. An immune cell delivery lipid nanoparticle comprising:
(i) an ionizable lipid;
(ii) a sterol or other structural lipid;
(iii) a non-cationic helper lipid or phospholipid;
(iv) an RNA interference agent, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to an immune cell.

2. An immune cell delivery lipid nanoparticle comprising:
(i) an ionizable lipid;
(ii) a sterol or other structural lipid;
(iii) a non-cationic helper lipid or phospholipid;
(iv) a PEG-lipid, and
(v) an RNA interference agent,
wherein one or more of (i) the ionizable lipid or (ii) the sterol or other structural lipid comprises an immune cell delivery potentiating lipid in an amount effective to enhance delivery of the lipid nanoparticle to an immune cell.

3. An immune cell delivery lipid nanoparticle comprising:
(i) an ionizable lipid;
(ii) a sterol or other structural lipid;
(iii) a non-cationic helper lipid or phospholipid;
(iv) an RNA interference agent, and
(v) optionally, a PEG-lipid
wherein one or more of (i) the ionizable lipid or (ii) the sterol or other structural lipid or (iii) the non-cationic helper lipid or phospholipid or (v) the PEG lipid is a C1q binding lipid that binds to C1q and/or promotes the binding of the LNP to C1q, as compared to a lipid nanoparticle lacking the C1q binding lipid.

4. The immune cell delivery lipid nanoparticle of any one of claims 1-3, wherein the enhanced delivery is relative to a lipid nanoparticle lacking the immune cell delivery potentiating lipid.

5. The immune cell delivery lipid nanoparticle of any one of claims 1-3, wherein the enhanced delivery is relative to a suitable control.

6. The immune cell delivery lipid nanoparticle of any one of claims 1-5, wherein the agent is an siRNA.

7. The immune cell delivery lipid nanoparticle of any one of claims 1-5, wherein the agent is an miRNA.

8. The immune cell delivery lipid nanoparticle of any one of claims 1-5, wherein the agent inhibits expression of a soluble protein that modulates immune cell activity.

9. The immune cell delivery lipid nanoparticle of any one of claims 1-5, wherein the agent inhibits expression of an intracellular protein that modulates immune cell activity.

10. The immune cell delivery lipid nanoparticle of any one of claims 1-5, wherein the agent inhibits expression of a transmembrane protein that modulates immune cell activity.

11. The immune cell delivery lipid nanoparticle of any one of claims 1-10, wherein the agent enhances immune function.

12. The immune cell delivery lipid nanoparticle of any one of claims 1-10, wherein the agent inhibits immune function.

13. The immune cell delivery lipid nanoparticle of any one of claims 1-12, wherein the immune cell is a T cell.

14. The immune cell delivery lipid nanoparticle of any one of claims 1-12, wherein the immune cell is a B cell.

15. The immune cell delivery lipid nanoparticle of any one of claims 1-12, wherein the immune cell is an NK cell, dendritic cell, myeloid cell or macrophage.

16. The immune cell delivery lipid nanoparticle of any one of claims 1-15, which comprises a phytosterol or a combination of a phytosterol and cholesterol.

17. The immune cell delivery lipid nanoparticle of claim 16, wherein the phytosterol is selected from the group consisting of β-sitosterol, stigmasterol, β-sitostanol, campesterol, brassicasterol, and combinations thereof.

18. The immune cell delivery lipid nanoparticle of claim 16, wherein the phytosterol comprises a sitosterol or a salt or an ester thereof.

19. The immune cell delivery lipid nanoparticle of claim 16, wherein the phytosterol comprises a stigmasterol or a salt or an ester thereof.

20. The immune cell delivery lipid nanoparticle of claim 16, wherein the phytosterol is beta-sitosterol 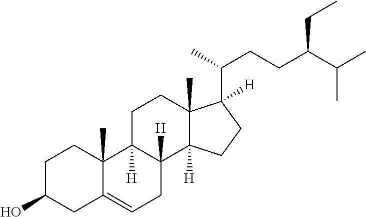
or a salt or an ester thereof.

21. The immune cell delivery lipid nanoparticle of any one of claims 1-20, which comprises a phytosterol, or a salt or ester thereof, and cholesterol or a salt thereof.

22. The immune cell delivery lipid nanoparticle of claim 21, wherein the immune cell is a T cell and the phytosterol or a salt or ester thereof is selected from the group consisting of β-sitosterol, β-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof.

23. The immune cell delivery lipid nanoparticle of claim 22, wherein the phytosterol is β-sitosterol.

24. The immune cell delivery lipid nanoparticle of claim 22, wherein the phytosterol is β-sitostanol.

25. The immune cell delivery lipid nanoparticle of claim 22, wherein the phytosterol is campesterol.

26. The immune cell delivery lipid nanoparticle of claim 22, wherein the phytosterol is brassicasterol.

27. The immune cell delivery lipid nanoparticle of claim 21, wherein the immune cell is a monocyte or a myeloid cell and the phytosterol or a salt or ester thereof is selected from the group consisting of β-sitosterol, and stigmasterol, and combinations thereof.

28. The immune cell delivery lipid nanoparticle of claim 27, wherein the phytosterol is β-sitosterol.

29. The immune cell delivery lipid nanoparticle of claim 27, wherein the phytosterol is stigmasterol.

30. The immune cell delivery lipid nanoparticle of any one of claims 1-20, which comprises a sterol, or a salt or ester thereof, and cholesterol, wherein the immune cell is a monocyte or a myeloid cell and the sterol or a salt or ester thereof is selected from the group consisting of brassicasterol, Compound S-30, Compound S-31 and Compound S-32.

31. The immune cell delivery lipid nanoparticle of claim 21, wherein the mol % cholesterol is between about 1% and 50% of the mol % of phytosterol present in the lipid nanoparticle.

32. The immune cell delivery lipid nanoparticle of claim 21, wherein the mol % cholesterol is between about 10% and 40% of the mol % of phytosterol present in the lipid nanoparticle.

33. The immune cell delivery lipid nanoparticle of claim 21, wherein the mol % cholesterol is between about 20% and 30% of the mol % of phytosterol present in the lipid nanoparticle.

34. The immune cell delivery lipid nanoparticle of claim 21, wherein the mol % cholesterol is about 30% of the mol % of phytosterol present in the lipid nanoparticle.

35. The immune cell delivery lipid nanoparticle of any one of claims 1-34, wherein the ionizable lipid comprises a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8).

36. The immune cell delivery lipid nanoparticle of any one of claims 1-34, wherein the ionizable lipid comprises a compound selected from the group consisting of Compound X, Compound Y, Compound I-48, Compound I-50, Compound I-109, Compound I-111, Compound I-113, Compound I-181, Compound I-182, Compound I-244, Compound I-292, Compound I-301, Compound I-309, Compound I-317, Compound I-321, Compound I-322, Compound I-326, Compound I-328, Compound I-330, Compound I-331, Compound I-332, Compound I-347, Compound I-348, Compound I-349, Compound I-350, Compound I-352 and Compound I-M.

37. The immune cell delivery lipid nanoparticle of any one of claims 1-34, wherein the ionizable lipid comprises a compound selected from the group consisting of Compound X, Compound Y, Compound I-321, Compound I-292, Compound I-326, Compound I-182, Compound I-301, Compound I-48, Compound I-50, Compound I-328, Compound I-330, Compound I-109, Compound I-111 and Compound I-181.

38. The immune cell delivery lipid nanoparticle of claim 37, wherein immune cell is a T cell.

39. The immune cell delivery lipid nanoparticle of claim 37, wherein immune cell is a T cell and the ionizable lipid comprises a compound selected from the group consisting of Compound I-301, Compound I-321, and Compound I-326.

40. The immune cell delivery lipid nanoparticle of claim 36, wherein immune cell is a monocyte or a myeloid cell and the ionizable lipid comprises a compound selected from the group consisting of Compound X, Compound I-109, Compound I-111, Compound I-181, Compound I-182, and Compound I-244.

41. The immune cell delivery lipid nanoparticle of any one of claims 1-40, wherein the non-cationic helper lipid or phospholipid comprises a compound selected from the group consisting of DSPC, DPPC, DMPC, DMPE, DOPC, Compound H-409, Compound H-418, Compound H-420, Compound H-421 and Compound H-422.

42. The immune cell delivery lipid nanoparticle of claim 41, wherein the phospholipid is DSPC.

43. The immune cell delivery lipid nanoparticle of claim 41, wherein the immune cell is a T cell and the non-cationic helper lipid or phospholipid comprises a compound selected from the group consisting of DSPC, DMPE, and Compound H-409.

44. The immune cell delivery lipid nanoparticle of claim 43, wherein the phospholipid is DSPC.

45. The immune cell delivery lipid nanoparticle of claim 43, wherein the phospholipid is DMPE.

46. The immune cell delivery lipid nanoparticle of claim 43, wherein the phospholipid is Compound H-409.

47. The immune cell delivery lipid nanoparticle of claim 41, wherein the immune cell is a monocyte or a myeloid cell and the non-cationic helper lipid or phospholipid comprises a compound selected from the group consisting of DOPC, DMPE, and Compound H-409.

48. The immune cell delivery lipid nanoparticle of claim 47, wherein the phospholipid is DOPC.

49. The immune cell delivery lipid nanoparticle of claim 47, wherein the phospholipid is DMPE.

50. The immune cell delivery lipid nanoparticle of claim 47, wherein the phospholipid is Compound H-409.

51. The immune cell delivery lipid nanoparticle of any one of claims 1-50, which comprises a PEG-lipid.

52. The immune cell delivery lipid nanoparticle of claim 51, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

53. The immune cell delivery lipid nanoparticle of claim 51, wherein the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L3, Compound P-L4, Compound P-L6, Compound P-L8, Compound P-L9, Compound P-L16, Compound P-L17, Compound P-L18, Compound P-L19, Compound P-L22, Compound P-L23 and Compound P-L25.

54. The immune cell delivery lipid nanoparticle of claim 53, wherein the immune cell is a T cell.

55. The immune cell delivery lipid nanoparticle of claim 51, wherein the PEG lipid comprises a compound selected from the group consisting of Compound P-428, Compound P-L16, Compound P-L17, Compound P-L18, Compound P-L19, Compound P-L1, and Compound P-L2.

56. The immune cell delivery lipid nanoparticle of any one of claims 1-55, which comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid.

57. The immune cell delivery lipid nanoparticle of any one of claims 1-55, which comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid.

58. The immune cell delivery lipid nanoparticle of any one of claims 1-55, which comprises about 50 mol % ionizable lipid, about 10 mol % non-cationic helper lipid or phospholipid, about 38.5 mol % sterol or other structural lipid, and about 1.5 mol % PEG lipid.

59. The immune cell delivery lipid nanoparticle of any one of claims 56-58, wherein the mol % sterol or other structural lipid is 18.5% phytosterol and the total mol % structural lipid is 38.5%.

60. The immune cell delivery lipid nanoparticle of any one of claims 56-58, wherein the mol % sterol or other structural lipid is 28.5% phytosterol and the total mol % structural lipid is 38.5%.

61. The immune cell delivery lipid nanoparticle of claim 59 or 60, wherein the immune cell is a T cell.

62. The immune cell delivery lipid nanoparticle of any one of claims 1-55, which comprises:
(i) about 50 mol % ionizable lipid, wherein the ionizable lipid is a compound selected from the group consisting of Compound I-301, Compound I-321, and Compound I-326;
(ii) about 10 mol % phospholipid, wherein the phospholipid is DSPC;
(iii) about 38.5 mol % structural lipid, wherein the structural lipid is selected from β-sitosterol and cholesterol; and
(iv) about 1.5 mol % PEG lipid, wherein the PEG lipid is Compound P-428.

63. The immune cell delivery lipid nanoparticle of any one of claims 1-62, wherein the RNA interference agent comprises at least one modified nucleobase, nucleoside and/or nucleotide.

64. The immune cell delivery lipid nanoparticle of any one of claims 1-63, wherein the immune cell is a Treg cell.

65. The immune cell delivery lipid nanoparticle of claim 64, wherein the RNA interference agent is an siRNA.

66. The immune cell delivery lipid nanoparticle of claim 65, wherein the siRNA targets an mRNA encoding Foxp3.

67. The immune cell delivery lipid nanoparticle of claim 64, wherein the RNA interference agent targets an mRNA encoding a protein selected from the group consisting of Foxp3, IRF4, estrogen receptor 1, HDAC6, HDAC10, HDAC11 and AEP.

68. The immune cell delivery lipid nanoparticle of claim 64, wherein the RNA interference agent targets miR-146b or anti-miR-146b.

69. The immune cell delivery lipid nanoparticle of any one of claims 1-63, wherein the immune cell is a Teff cell.

70. The immune cell delivery lipid nanoparticle of claim 69, wherein the RNA interference agent is an siRNA.

71. The immune cell delivery lipid nanoparticle of claim 69, wherein the Teff cell is a Th17 cell.

72. The immune cell delivery lipid nanoparticle of claim 71, wherein the RNA interference agent is an siRNA that targets an mRNA encoding RORγt or IL-17a.

73. The immune cell delivery lipid nanoparticle of claim 69, wherein the RNA interference agent targets an mRNA encoding a protein selected from the group consisting of RORγt, IL-17a, Tbet, Kv1.3, KCA3.1 and KCNNA.

74. A method of delivering an agent to an immune cell, the method comprising contacting the immune cell with an immune cell delivery lipid nanoparticle comprising:
(i) an ionizable lipid;
(ii) a sterol or other structural lipid;
(iii) a non-cationic helper lipid or phospholipid;
(iv an RNA interference agent, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to an immune cell,
such that the agent is delivered to the immune cell.

75. A method of modulating T cell activation or activity, the method comprising contacting a T cell with an immune cell delivery lipid nanoparticle comprising:
(i) an ionizable lipid;
(ii) a sterol or other structural lipid;
(iii) a non-cationic helper lipid or phospholipid;
(iv) an RNA interference agent, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to a T cell,
such that T cell activation or activity is modulated.

76. The method of claim 75, wherein the T cell is a Treg cell.

77. The method of claim 75, wherein the T cell is a Teff cell.

78. The method of claim 77, wherein the Teff cell is a Th17 cell.

79. A method of increasing an immune response to a protein, the method comprising contacting immune cells with an immune cell delivery lipid nanoparticle comprising:
(i) an ionizable lipid;
(ii) a sterol or other structural lipid;
(iii) a non-cationic helper lipid or phospholipid;
(iv) an RNA interference agent, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to immune cells,
such that the immune response to the protein is increased.

80. A method of increasing a T cell response to a cancer antigen, the method comprising contacting the T cell with an immune cell delivery lipid nanoparticle comprising:
(i) an ionizable lipid;
(ii) a sterol or other structural lipid;
(iii) a non-cationic helper lipid or phospholipid;
(iv) an RNA interference agent, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to immune cells,
such that the T cell response to the cancer antigen is increased.

81. A method of modulating an immune response in a subject, the method comprising administering to the subject an immune cell delivery lipid nanoparticle comprising:
(i) an ionizable lipid;
(ii) a sterol or other structural lipid;
(iii) a non-cationic helper lipid or phospholipid;
(iv) an RNA interference agent, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to immune cells,
such that an immune response is modulated in the subject.

82. A method of modulating B cell activation or activity, the method comprising contacting a B cell with an immune cell delivery lipid nanoparticle comprising:
(i) an ionizable lipid;
(ii) a sterol or other structural lipid;
(iii) a non-cationic helper lipid or phospholipid;
(iv) an RNA interference agent, and
(v) optionally, a PEG-lipid
wherein one or more of (i) the ionizable lipid or (ii) the sterol or other structural lipid comprises an immune cell delivery potentiating lipid in an amount effective to enhance delivery of the lipid nanoparticle to immune cells,
such that B cell activation or activity is modulated.

83. The method of any one of claims 74-82, wherein the enhanced delivery is relative to a lipid nanoparticle lacking the immune cell delivery potentiating lipid.

84. The method of any one of claims 74-82, wherein the enhanced delivery is relative to a suitable control.

85. The method of any one of claims 74-82, wherein the RNA interference agent is an siRNA

86. The method of any one of claims 74-82, wherein the RNA interference agent is an miRNA.

87. The method of any one of claims 74-82, wherein the RNA interference agent inhibits expression of a soluble protein that modulates immune cell activity.

88. The method of any one of claims 74-82, wherein the RNA interference agent inhibits expression of an intracellular protein that modulates immune cell activity.

89. The method of any one of claims 74-82, wherein the RNA interference agent inhibits expression of a transmembrane protein that modulates immune cell activity.

90. The method of any one of claims 74-89, wherein the RNA interference agent enhances immune function.

91. The method of any one of claims 74-89, wherein the RNA interference agent inhibits immune function.

92. The method of any one of claims 74-91, wherein the immune cell is a T cell.

93. The method of any one of claims 74-91, wherein the immune cell is a B cell.

94. The method of any one of claims 74-91, wherein the immune cell is an NK cell, a dendritic cell, a myeloid cell or a macrophage.

95. The method of any one of claims 74-94, wherein the LNP comprises a phytosterol or a combination of a phytosterol and cholesterol.

96. The method of claim 95, wherein the phytosterol is selected from the group consisting of β-sitosterol, stigmasterol, β-sitostanol, campesterol, brassicasterol, and combinations thereof.

97. The method of claim 95, wherein the phytosterol comprises a sitosterol or a salt or an ester thereof.

98. The method of claim 95, wherein the phytosterol comprises a stigmasterol or a salt or an ester thereof.

99. The method of claim 95, wherein the phytosterol is beta-sitosterol 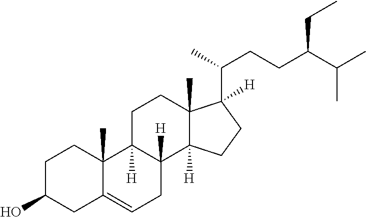
HO or a salt or an ester thereof.

100. The method of any one of claims 74-99, wherein the LNP comprises a phytosterol, or a salt or ester thereof, and cholesterol or a salt thereof.

101. The method of claim 100, wherein the immune cell is a T cell and the phytosterol or a salt or ester thereof is selected from the group consisting of β-sitosterol, β-sitostanol, campesterol, brassicasterol, and combination thereof.

102. The method of claim 101, wherein the phytosterol is β-sitosterol.

103. The method of claim 101, wherein the phytosterol is β-sitostanol.

104. The method of claim 101, wherein the phytosterol is campesterol.

105. The method of claim 101, wherein the phytosterol is brassicasterol.

106. The method of claim 100, wherein the immune cell is a monocyte or a myeloid cell and the phytosterol or a salt or ester thereof is selected from the group consisting of β-sitosterol, stigmasterol, and combinations thereof.

107. The method of claim 106, wherein the phytosterol is β-sitosterol.

108. The method of claim 106, wherein the phytosterol is stigmasterol.

109. The method of any one of claims 74, 79, 81 and 83-91, wherein the LNP comprises a sterol, or a salt or ester thereof, and cholesterol, and wherein the immune cell is a monocyte or a myeloid cell and the sterol or a salt or ester thereof is selected from the group consisting of brassicasterol, Compound S-30, Compound S-31 and Compound S-32.

110. The method of claim 100, wherein the mol % cholesterol is between about 1% and 50% of the mol % of phytosterol present in the lipid nanoparticle.

111. The method of claim 100, wherein the mol % cholesterol is between about 10% and 40% of the mol % of phytosterol present in the lipid nanoparticle.

112. The method of claim 100, wherein the mol % cholesterol is between about 20% and 30% of the mol % of phytosterol present in the lipid nanoparticle.

113. The method of claim 100, wherein the mol % cholesterol is about 30% of the mol % of phytosterol present in the lipid nanoparticle.

114. The method of any one of claims 74-113, wherein the LNP comprises an ionizable lipid comprising a compound of any of Formulae (I I), (I IA), (I IB), (I II), (I IIa), (I IIb), (I IIc), (I IId), (I IIe), (I IIf), (I IIg), (I III), (I VI), (I VI-a), (I VII), (I VIII), (I VIIa), (I VIIIa), (I VIIIb), (I VIIb-1), (I VIIb-2), (I VIIb-3), (I VIIc), (I VIId), (I VIIIc), (I VIIId), (I IX), (I IXa1), (I IXa2), (I IXa3), (I IXa4), (I IXa5), (I IXa6), (I IXa7), or (I IXa8) and/or any of Compounds X, Y, I 48, I 50, I 109, I 111, I 113, I 181, I 182, I 244, I 292, I 301, I 321, I 322, I 326, I 328, I 330, I 331, I 332 or I M.

115. The method of any one of claims 74-113, wherein the LNP comprises an ionizable lipid comprising a compound selected from the group consisting of Compound X, Compound Y, Compound I-48, Compound I-50, Compound I-109, Compound I-111, Compound I-113, Compound I-181, Compound I-182, Compound I-244, Compound I-292, Compound I-301, Compound I-321, Compound I-322, Compound I-326, Compound I-328, Compound I-330, Compound I-331, Compound I-332 and Compound I-M.

116. The method of any one of claims 74-113, wherein the LNP comprises an ionizable lipid comprising a compound selected from the group consisting of Compound X, Compound Y, Compound I-321, Compound I-292, Compound I-326, Compound I-182, Compound I-301, Compound I-48, Compound I-50, Compound I-328, Compound I-330, Compound I-109, Compound I-111 and Compound I-181.

117. The method of claim 116, wherein the immune cell is a T cell.

118. The method of claim 116, wherein the immune cell is a T cell and the ionizable lipid comprises a compound selected from the group consisting of Compound I-301, Compound I-321 and Compound I-326.

119. The method of claim 116, wherein the immune cell is a monocyte or a myeloid cell and the ionizable lipid comprises a compound selected from the group consisting of Compound X, Compound I-109, Compound I-111, Compound I-181, Compound I-182 and Compound I-244.

120. The method of any one of claims 74-119, wherein the LNP comprises a non-cationic helper lipid or phospholipid that comprises a compound selected from the group consisting of DSPC, DMPE, DOPC and Compound H-409.

121. The method of claim 120, wherein the phospholipid is DSPC.

122. The method of claim 120, wherein the immune cell is a T cell and the non-cationic helper lipid or phospholipid comprises a compound selected from the group consisting of DSPC, DMPE and Compound H-409.

123. The method of claim 122, wherein the phospholipid is DSPC.

124. The method of claim 122, wherein the phospholipid is DMPE.

125. The method of claim 122, wherein the phospholipid is Compound H-409.

126. The method of claim 119, wherein the immune cell is a monocyte or a myeloid cell and the non-cationic helper lipid or phospholipid comprises a compound selected from the group consisting of DOPC, DMPE and Compound H-409.

127. The method of claim 126, wherein the phospholipid is DOPC.

128. The method of claim 126, wherein the phospholipid is DMPE.

129. The method of claim 126, wherein the phospholipid is Compound H-409.

130. The method of any one of claims 74-129, wherein the LNP comprises a PEG-lipid.

131. The method of claim 130, wherein the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

132. The method of claim 130, wherein the PEG lipid comprises a compound selected from the group consisting of Compound P-415, Compound P-416, Compound P-417, Compound P-419, Compound P-420, Compound P-423, Compound P-424, Compound P-428, Compound P-L1, Compound P-L2, Compound P-L16, Compound P-L17, Compound P-L18, Compound P-L19, Compound P-L22 and Compound P-L23.

133. The method of claim 132, wherein the immune cell is a T cell.

134. The method of claim 130, wherein the PEG lipid comprises a compound selected from the group consisting of Compound P-428, Compound P-L16, Compound P-L17, Compound P-L18, Compound P-L19, Compound P-L1, and Compound P-L2.

135. The method of any one of claims 74-134, wherein the LNP comprises about 30 mol % to about 60 mol % ionizable lipid, about 0 mol % to about 30 mol % non-cationic helper lipid or phospholipid, about 18.5 mol % to about 48.5 mol % sterol or other structural lipid, and about 0 mol % to about 10 mol % PEG lipid.

136. The method of any one of claims 74-134, wherein the LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid.

137. The method of any one of claims 74-134, wherein the LNP comprises about 50 mol % ionizable lipid, about 10 mol % non-cationic helper lipid or phospholipid, about 38.5 mol % sterol or other structural lipid, and about 1.5 mol % PEG lipid.

138. The method of any one of claims 74-134, wherein the mol % sterol or other structural lipid is 18.5% phytosterol and the total mol % structural lipid is 38.5%.

139. The method of any one of claims 74-134, wherein the mol % sterol or other structural lipid is 28.5% phytosterol and the total mol % structural lipid is 38.5%.

140. The method of claim 138 or 139, wherein the immune cell is a T cell.

141. The method of any one of claims 74-134, wherein the LNP comprises
(i) about 50 mol % ionizable lipid, wherein the ionizable lipid is a compound selected form the group consisting of Compound I-301, Compound I-321 and Compound I-326;
(ii) about 10 mol % phospholipid, wherein the phospholipid is DSPC;
(iii) about 38.5 mol % structural lipid, wherein the structural lipid is selected from β-sitosterol and cholesterol; and
(iv) about 1.5 mol % PEG lipid, wherein the PEG lipid is Compound P-428.

142. The method of any one of claims 74-141, wherein the RNA interference agent comprises at least one modified nucleobase, nucleoside and/or nucleotide.

143. The method of any one of claims 74, 75 or 81, wherein the immune cell is a Treg cell.

144. The method of claim 143, wherein the RNA interference agent is an siRNA.

145. The method of claim 144, wherein the siRNA targets an mRNA encoding Foxp3.

146. The method of claim 143, wherein the RNA interference agent targets an mRNA encoding a protein selected from the group consisting of Foxp3, IRF4, estrogen receptor 1, HDAC6, HDAC10, HDAC11 and AEP.

147. The method of claim 143, wherein the RNA interference agent targets miR-146b or anti-miR-146b.

148. The method of any one of claims 74, 75 or 81, wherein the immune cell is a Teff cell.

149. The method of claim 148, wherein the RNA interference agent is an siRNA.

150. The method of claim 148, wherein the Teff cell is a Th17 cell.

151. The method of claim 150, wherein the RNA interference agent is an siRNA that targets an mRNA encoding RORγt or IL-17a.

152. The method of claim 148, wherein the RNA interference agent targets an mRNA encoding a protein selected from the group consisting of RORγt, IL-17a, Tbet, Kv1.3, KCA3.1 and KCNNA.

153. A pharmaceutical composition comprising the immune cell delivery lipid nanoparticle of any one of claims 1-73, and a pharmaceutically acceptable carrier.

154. A kit comprising a container comprising the immune cell delivery lipid nanoparticle of any one of claims 1-73, or the pharmaceutical composition of claim 153, and a package insert comprising instructions for administration of the lipid nanoparticle or pharmaceutical composition for modulating an immune response in an individual.

155. The immune cell delivery lipid nanoparticle of any one of claims 1-73, or the pharmaceutical composition of claim 153, for use in modulating an immune response in an individual.

156. The immune cell delivery lipid nanoparticle of any one of claims 1-73, or the pharmaceutical composition of claim 153, for use in delivering an agent to an immune cell in an individual.

157. The immune cell delivery lipid nanoparticle of any one of claims 1-73, or the pharmaceutical composition of claim 153, for use in modulating T cell activity or activation in an individual.

158. The immune cell delivery lipid nanoparticle of any one of claims 1-73, or the pharmaceutical composition of claim 153, for use in increasing an immune response to a protein in an individual.

159. The immune cell delivery lipid nanoparticle of any one of claims 1-73, or the pharmaceutical composition of claim 153, for use in increasing a T cell response to a cancer antigen in an individual.

160. The immune cell delivery lipid nanoparticle of any one of claims 1-73, or the pharmaceutical composition of claim 153, for use in modulating B cell activity or activation in an individual.
